(19) United States
(12) Patent Application Publication
(10) Pub. No.: US 2024/0115557 A1
(43) Pub. Date: Apr. 11, 2024

(54) INHIBITORS OF RNA-GUIDED NUCLEASES AND USES THEREOF

(71) Applicants: The Brigham and Women's Hospital, Inc., Boston, MA (US); The Broad Institute, Inc., Cambridge, MA (US)

(72) Inventors: Amit Choudhary, Boston, MA (US); Donghyun Lim, Cambridge, MA (US); Qingxuan Zhou, Boston, MA (US); Kurt Cox, Cambridge, MA (US); Benjamin Law, Cambridge, MA (US); Miseon Lee, Cambridge, MA (US); Praveen Kokkonda, Boston, MA (US); Sreekanth Vedagopuram, Boston, MA (US)

(21) Appl. No.: 18/361,352

(22) Filed: Jul. 28, 2023

Related U.S. Application Data
(60) Provisional application No. 63/393,788, filed on Jul. 29, 2022.

(51) Int. Cl. A61K 31/4439

(52) U.S. Cl. CPC A61K 31/4439; C12Y 301/22001

(57) ABSTRACT

The need to control the activity and fidelity of CRISPR-associated nucleases has resulted in the demand for inhibitory anti-CRISPR molecules. Current small-molecule inhibitor discovery platforms are not generalizable to multiple nuclease classes, only target the initial step in the catalytic activity, and require high concentration of nuclease, resulting in inhibitors with suboptimal attributes, including poor potency. Herein, Applicants report a high-throughput discovery pipeline consisting of a FRET-based assay that is generalizable to contemporary and emerging nucleases, operates at low nuclease concentration, and targets all catalytic steps. Applicants applied this pipeline to identify BRD7586, a cell-permeable small-molecule inhibitor of SpCas9, that is 2-fold more potent than current inhibitors. Furthermore, unlike the reported inhibitors, BRD7586 enhanced SpCas9 specificity and its activity was independent of the genomic loci, DNA repair pathway, or mode of nuclease delivery. Overall, these studies describe a general pipeline to identify inhibitors of contemporary and emerging CRISPR-associated nucleases. Described herein are compositions and methods for inhibiting the activity of RNA-guided endonucleases, and methods for identifying such compositions.

### CROSS-REFERENCE TO RELATED APPLICATIONS

This application claims the benefit of U.S. Provisional Application No. 63/393,788, filed Jul. 29, 2022. The entire contents of the above-identified applications are hereby fully incorporated herein by reference.

### STATEMENT REGARDING FEDERALLY SPONSORED RESEARCH

This invention was made with government support under DARPA N66001-17-2-4055 awarded by the Defense Advanced Research Projects Agency. The government has certain rights in the invention.

### REFERENCE TO AN ELECTRONIC SEQUENCE LISTING

The contents of the electronic sequence listing (“BROD-5655US_ST26.xml”; Size is 75,271 bytes and it was created on Nov. 14, 2023) is herein incorporated by reference in its entirety.

### TECHNICAL FIELD

The subject matter disclosed herein is generally directed to Cas inhibitors and methods of inhibiting engineered Cas systems.

### BACKGROUND

The CRISPR (clustered regularly interspaced short palindromic repeat) system is an adaptive immune system used by bacteria and archaea to defend against invading phages or mobile genetic elements. The most studied CRISPR system employs an RNA-guided endonuclease Cas9, which can cleave double-stranded target DNA in multiple cell types. Two common variants of Cas9 are SpCas9 and SaCas9, which naturally occur in Streptococcus pyogenes and Staphylococcus aureus, respectively, and recently another endonuclease called Cpf1 has been reported. The relative ease of targeting Cas9/Cpf1 to specific genomic loci has enabled the development of revolutionary biomedical technologies.

While CRISPR-Cas has emerged as a powerful tool in the field of biotechnology, high CRISPR activity has been known to cause off-target gene editing effects. Dose control and temporal control of CRISPR-based gene drives is also desirable, particularly for in vivo applications. Gene drives enable replacement of one version of the gene with the other “selfish” version of the gene, thereby converting a heterozygous individual to homozygous individual. In laboratory settings, CRISPR-based gene drives have successfully enabled self-propagation of engineered genes in multiple organisms (e.g., mosquitoes) and complete annihilation of wild-type genes. Cas-based technologies (e.g., transcriptional regulation) would benefit from dosable and temporal control of Cas activity. Inhibitors of CRISPR-Cas could also emerge as a novel class of antibiotics that disrupt CRISPR-immunity of bacteria from phage.

Reports of small-molecule controlled Cas9 activity are present in literature and involve fusing Cas9 to small-molecule controlled protein domains. Genetic-fusions of Cas9 to small-molecule controlled degrons (e.g., Wandless' destabilized domains) may allow aforementioned controls, but such fusions have unacceptably high background activity presumably owing to the large size of Cas9. These systems also do not ensure dosage control; the small molecules act merely as an inducer of Cas9 activity. Further, these “inducer” small molecules cannot control gene drives containing wild-type Cas9/Cpf1. A general approach would be desirable to control all variants of Cas9/Cpf1, including the wild type and engineered versions. The use of “inducible” systems to control gene drives is also questionable given that the “inducer” small molecules are toxic at the organismal level (albeit not at the cellular level, where these systems were developed).

A need exists for compositions and methods for inhibiting one or more activities of RNA-guided nuclease (e.g., Cas9, Cpf1). Such compositions and methods are useful for regulating the activity of RNA-guided nucleases (e.g., in genome editing).

### SUMMARY

In certain example embodiments, methods for inhibiting an RNA-guided nuclease are provided. A method of inhibiting an activity of an RNA-guided endonuclease comprises contacting the RNA-guided endonuclease with the compound of any one of Tables 1-6; a compound of formula (I)

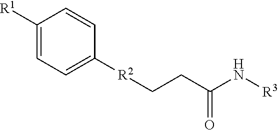

wherein R¹, R², and R³ are independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof; or a compound selected from the group consisting of

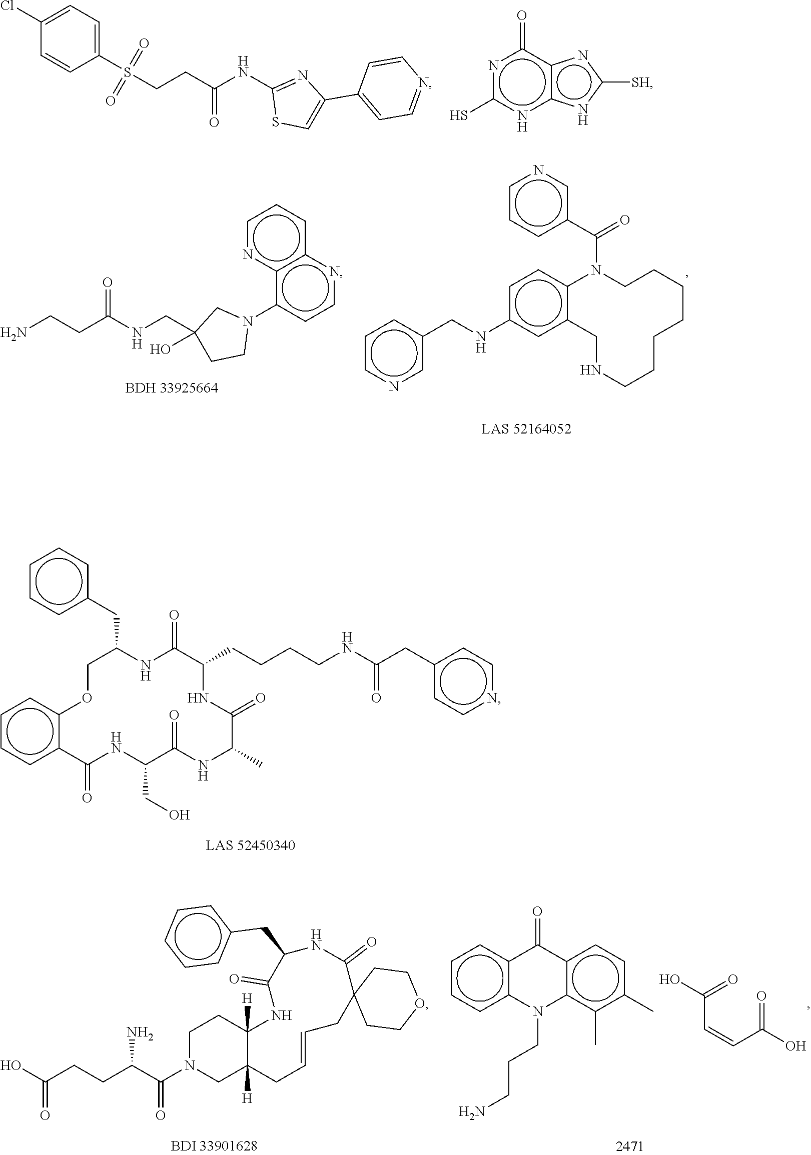

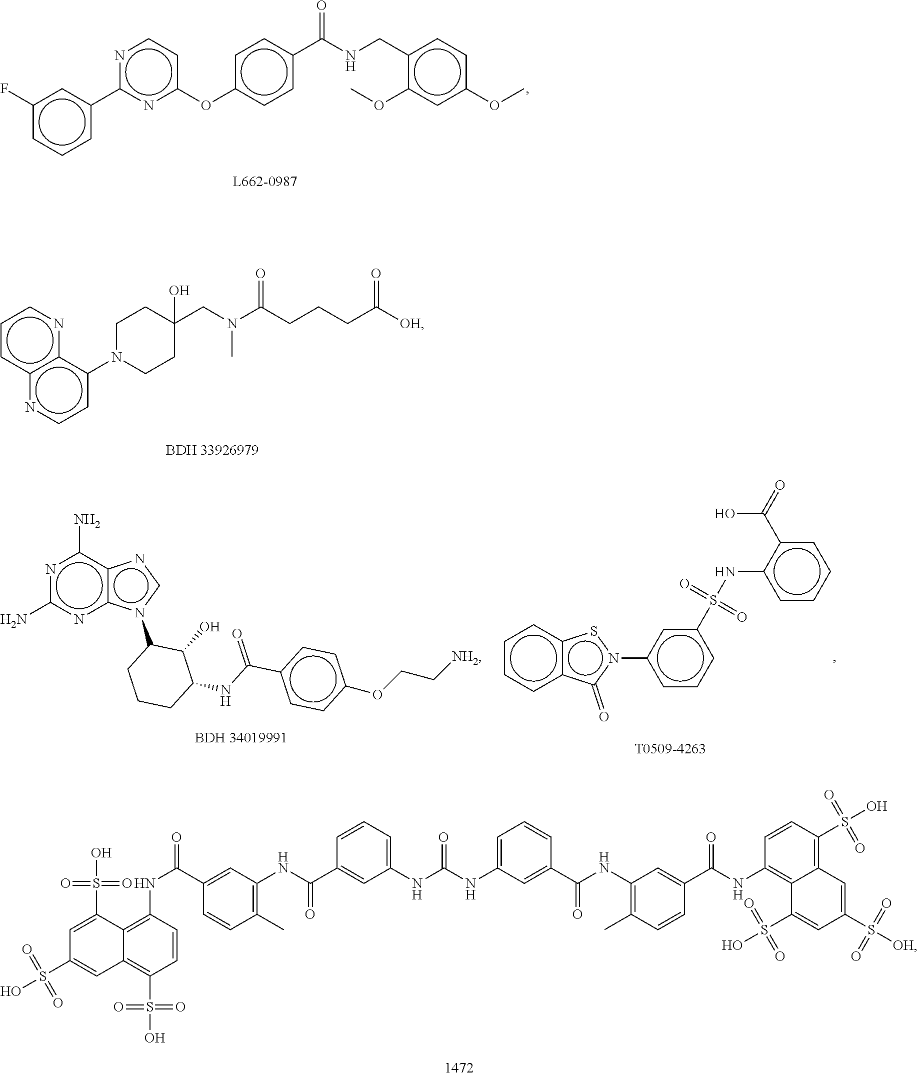

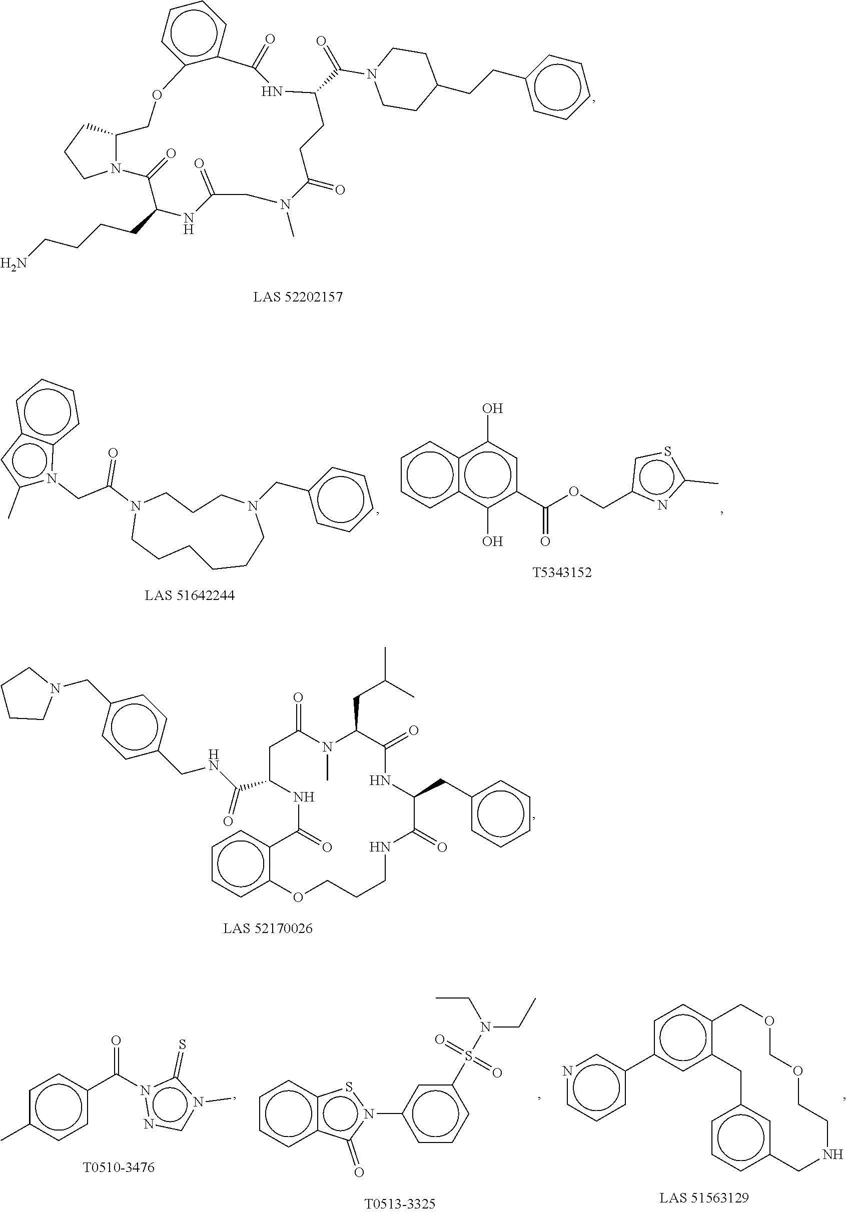

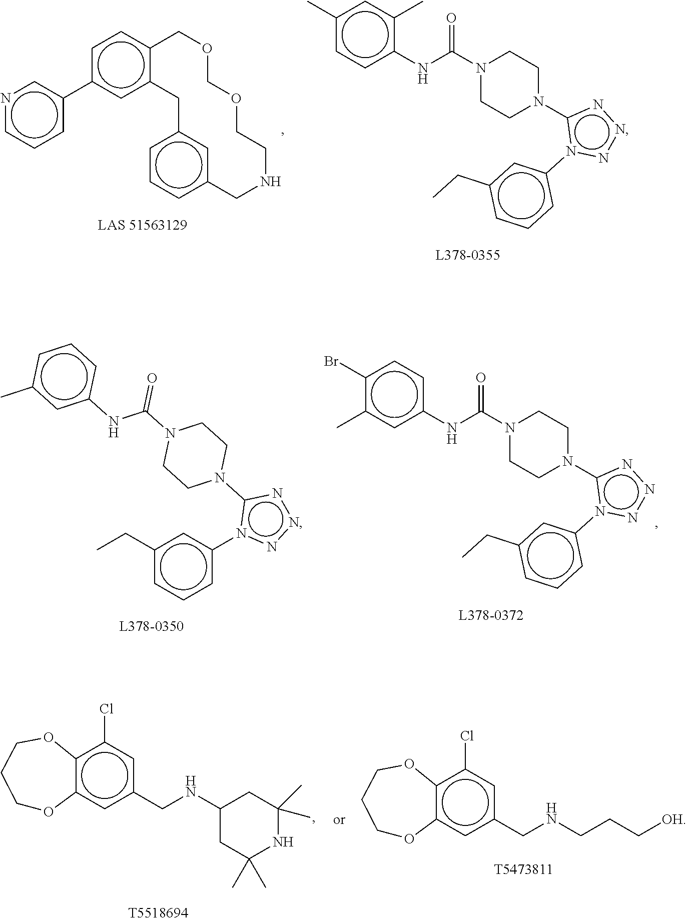

In example embodiment, the inhibitor is the compound of formula I and R¹ is Cl, H, F, or OMe; R² is

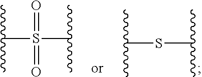

### R³ is

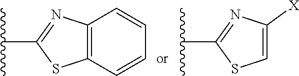

wherein X is independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof. In example embodiments, X is

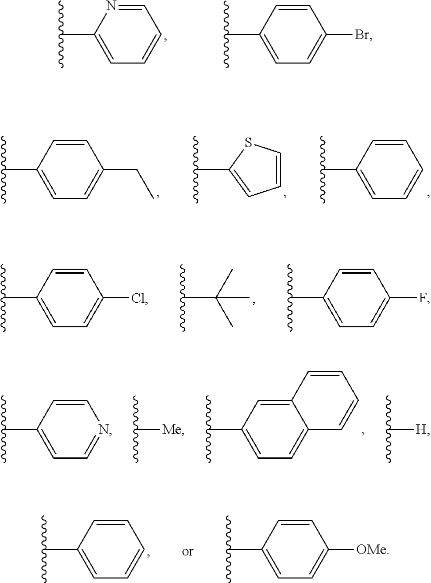

In example embodiments, the inhibitor is a compound of formula II

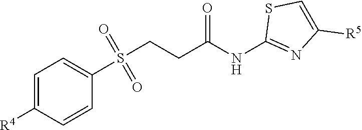

R⁴ and R⁵ are independently selected from a hydrogen, alkane, alkene, alkyne, ether, alcohol, amine, nitrile, nitro, thiol, sulfone, sulfonate, halogen, carbonyl; acyl; ketone; carboxylate ester, amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof. In an example embodiment, the inhibitor is the compound of formula II and R⁴ is H, F, Cl, OH, Me, or OMe and R⁵ is

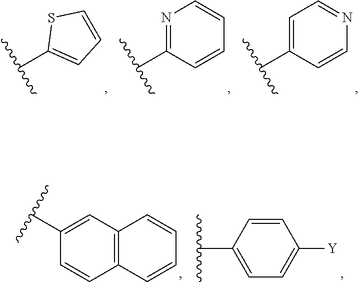

wherein Y is selected from substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen. In example embodiments, R⁵ is

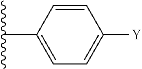

and wherein Y is selected from F, Cl, Br, I, OMe, or Et. In example embodiments, the method of inhibiting an activity of an RNA-guided endonuclease comprises contacting the RNA-guided endonuclease with the compound

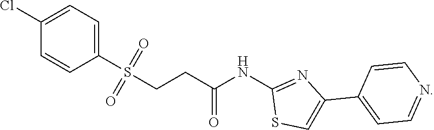 [SMILES: O=C(CCS(=O)(=O)c1ccc(Cl)cc1)Nc1nc(-c2ccncc2)cs1]

The methods as provided herein can inhibit the activity of an RNA-guided endonuclease reversibly. The methods can be performed in vitro or in vivo. In one aspect, the method is performed in a cell. The cell can be a germline cell. In embodiments, the cell is a prokaryotic cell, which can be a bacterium. In embodiments, the cell is a eukaryotic cell. In some instances, the eukaryotic cell is a human cell, a mammalian cell, an insect cell, a plant cell, or a yeast cell. The cell can in certain embodiments be in an organism, which may be a human, mammal, vertebrate, invertebrate, insect, or plant.

In embodiments, the RNA-guided endonuclease is Cas9. In some embodiments, the RNA-guided endonuclease is Streptococcus pyogenes Cas9 or a variant thereof. In example embodiments, the RNA-guided endonuclease is Staphylococcus aureus Cas 9 (SaCas9).

In some embodiments, a method of treating a subject is provided, comprising administering an RNA-guided endonuclease-RNA complex or a reagent causing expression of the RNA-guided endonuclease-RNA complex to the subject; and administering an effective amount of a compound as defined herein.

In one aspect, described herein, a RNA-guided endonuclease inhibitor comprising a compound of formula (I)

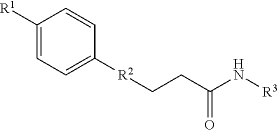

wherein R¹, R², and R³ are independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof.

In example embodiments, the inhibitor is the compound of formula I and R¹ is Cl, H, F, or OMe; R² is

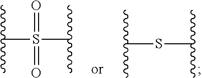

### R³ is

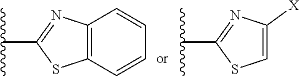

wherein X is independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or an combination of the groups previously mentioned thereof. In example embodiments, X is

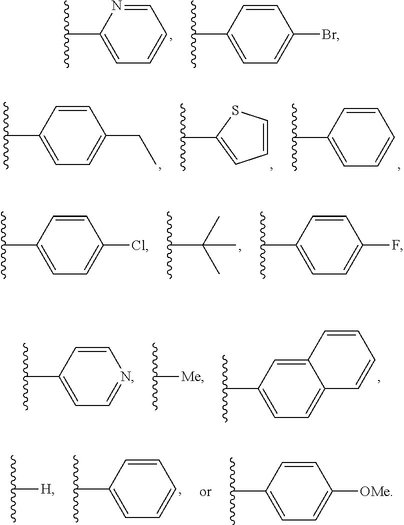

In example embodiments, the inhibitor is a compound of formula II

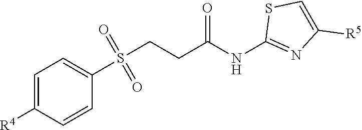

wherein R⁴ and R⁵ are independently selected from a hydrogen, alkane, alkene, alkyne, ether, alcohol, amine, nitrile, nitro, thiol, sulfone, sulfonate, halogen, carbonyl; acyl; ketone; carboxylate ester, amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof.

In example embodiments, the inhibitor is the compound of formula II and R⁴ is H, F, Cl, OH, Me, or OMe and R⁵ is

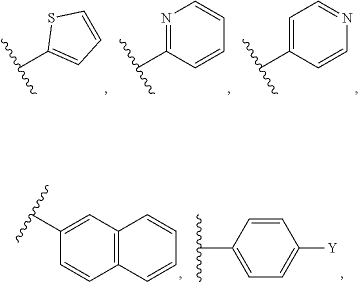

wherein Y is selected from substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen. In example embodiments, R⁵ is

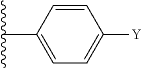

and wherein Y is selected from F, Cl, Br, I, OMe, or Et. In one aspect, described herein, in example embodiments, the RNA-guided endonuclease inhibitor is

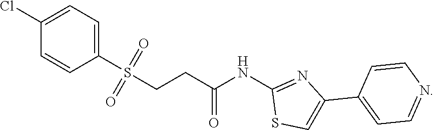 [SMILES: O=C(CCS(=O)(=O)c1ccc(Cl)cc1)Nc1nc(-c2ccncc2)cs1]

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—SpCas9 inhibitor compounds of interest and performance in SDA, eGFP disruption, inhibition in Hibit, and in surveyor assay using plasmid and RNP.

FIG. 2—Results of Cell Titer Glo Viability Assay for SpCas9 inhibitors conducted in U2OS cells.

FIG. 3—charts % PrestoBlue viability in HEK293 cell.

FIG. 4—scatterplot of SpCas9 inhibitors, identified as ‘Cpd’ in plot, and Table of preferred inhibitors based on GFP assay.

FIG. 5—graphs normalized percent inhibition of select SpCas9 inhibitors in eGFP disruption assay.

FIG. 6—Dose curve eGFP disruption for several SpCas9 inhibitors, with Efficacy: G786-1325>G786-1264>G786-1324>T5535170>T5461482, and Potency: G786-1325>T5535170>G786-1264>G786-1324>T5461482.

FIG. 7—Normalized inhibition in Hibit assay of select SpCas9 inhibitors, with Inhibition: G786-1325>G786-1324>G786-1264>T5535170>T5461482.

FIG. 8—includes gel from Surveyor_emx1 assay in HEK293T (plasmid) for select inhibitors, with Inhibition: T5535170>G786-1324>G786-1325>G786-1264>T5461482; inhibition from assay quantified in graph, right.

FIG. 9—includes gel from Surveyor assay_egfp in U2OS (Plasmid & RNP) for select inhibitors, with Inhibition: Plasmid: T5535170>T5461482>G786-1324; and with RNP: T5461482>T5535170>G786-1324; inhibition from assay quantified in graph, right.

FIG. 10—provides gels from Surveyor assay_egfp in U2OS (RNP assay).

FIG. 11—charts quantification of gels from surveyor assay_egfp from FIG. 10 with Inhibition at 15 uM: T535170>G786-1324>G786-1325/G786-1264/T5461482.

FIG. 12—scatterplot of potential inhibitors of SaCas9 from ChemDiv4 and Enamine 2 library.

FIG. 13—scatterplot of potential inhibitors of SaCas9 from ChemDiv4 and Enamine 2 library.

FIG. 14—Overview Oo SaCas9 screen pipeline.

FIG. 15—Scatterplot of ICCB Screening of Potential SaCas9 inhibitors.

FIG. 16—Determining the limit of detection for the SaCas9 DS-AF647 substrate in its quenched (SS-AF647+Q) and unquenched (DS-AF647+Q) forms. SS-AF647=single stranded DNA attached to AF647 that is complementary to the quencher strand (Q). DS-AF647=double stranded DNA duplex conjugated to AF647 that cannot bind to the quencher strand. (A) LOD determination using quencher strand labeled with Iowa Black@ RQ (RedQ). (B) LOD determination using a quencher strand labeled with Iowa Black@ FQ (FAMQ). In each case, Q was in 5-fold molar excess of the AF647-labeled DNA. 1 nM of DS-AF647 with using FAMQ gave the highest dynamic range with the best sensitivity.

FIG. 17—Cell based workflow for identifying FnCpf1 inhibitors. After retesting 210 compounds in cells, none were found to be active by the Surveyor assay.

FIG. 18—Determining the limit of detection for the FnCpf1DS-AF647 substrate in its quenched (SS-AF647+Q) and unquenched (DS-AF647+Q) forms. SS-AF647=single stranded DNA attached to AF647 that is complementary to the quencher strand (Q). DS-AF647=double stranded DNA duplex conjugated to AF647 that cannot bind to the quencher strand. (A) LOD determination using quencher strand labeled with Iowa Black@ RQ (RedQ). (B) LOD determination using a quencher strand labeled with Iowa Black@ FQ (FAMQ). In each case, Q was in 5-fold molar excess of the AF647-labeled DNA. 1 nM of DS-AF647 with using FAMQ gave the highest dynamic range with the best sensitivity.

FIG. 19—(A-B) Optimization of protein:DNA (DNA=DS-AF647) ratio for SaCas9 (A) and FnCpf1 (B). For SaCas9, a ratio of 1:5 DS-AF647:SaCas9 was optimal for screening, while that ratio was 1:10 for DS-AF647:FnCpf1. (C-D): Optimization of enzyme/DNA incubation time for SaCas9 (C) and FnCpf1 (D) at various protein:DNA ratios. 120 min was optimal for SaCas9, while 90 min was optimal for FnCpf1.

FIG. 20—Determining the optimal DNA:Quencher ratio for (A) SaCas9 and (B) FnCpf1 substrates. All proteins were used in 5-fold molar excess to their DNA substrate concentration. Incubation times were 120 min for SaCas9 and 90 min for FnCpf1. In each case, a ratio of 1:5 DNA to Quencher strand was found to be optimal.

FIG. 21—Compounds screened against FnCpf1 at ICCB (119,362 compounds total, left graph). After removing autofluorescent compounds (right graph), 263 compounds had activity >3 sigma over DMSO. These were further tested in gel cleavage assays.

FIG. 22—Examples of the gel cleavage assay used to identify active and specific inhibitors of FnCpf1. Inhibitors were screened at 20 uM against FnCpf1 under conditions identical to that used for the primary screening strand displacement assay (left gel). Inhibitors were also screened against the unrelated restriction endonuclease Nde1 to determine specificity (right gel). GreenL₃₇₈₋₀₃₅₀ L₃₇₈₋₀₃₅₅ L₃₇₈₋₀₃₇₂ boxes show inhibitors active in Fncpf1 but not Nde1, indicating potential specificity of target. Inhibitors here are L₃₇₈₋₀₃₅₀, L378-0355, and L378-0372, respectively.

FIG. 23—Summary of all gel cleavage data for the 3 s and select 2.8 s inhibitors affecting FnCpf1 and Nde1 activity as quantified by gel densitometry. The red box indicates compounds with >20% inhibition of FnCpf1 by gel cleavage. The green box indicates those molecules with an additional <20% inhibition of Nde1.

FIG. 24—Structures of all molecules+names with >20% inhibition of FnCpf1 by gel cleavage. Bolded molecules also show some specificity toward FnCpf1, as they have <20% inhibition of Nde1. Boxed molecules show some SAR.

FIG. 25—Example of the eGFP assay with potential inhibitors of FnCpf1 found from the triage process in FIG. 17.

FIG. 26A-26B—Exemplary strand displacement assay for detecting nuclease activity. FIG. 26A A fluorescence-based strand displacement assay (SDA) for monitoring Cas nuclease activity. Following cleavage, a fluorophore-bearing double stranded oligo (DS-fluor) is displaced by a quencher (Q)-bearing displacer strand (Disp-Q), resulting in a decrease in a fluorescence signal. FIG. 26B exemplary chart showing DS-fluor is not quenched in the presence of Disp-Q unless the duplex is disrupted by cleavage via an active Cas:gRNA complex (RNP).

FIG. 27A-27G—Optimizations of the SDA for SpCas9. FIG. 27A A PAM-sequence is required for quenching in the SDA; FIG. 27B Validation of SDA-substrate activity using a gel cleavage assay; FIG. 27C Optimizing the ratio of DS-fluor to SpCas9/gRNA; FIG. 27D Optimizing the ratio of DS-fluor to Q-oligo; FIG. 27E Time course of SpCas9/gRNA cleavage and substrate quenching at various DS-fluor; SpCas9/gRNA ratios; FIG. 27F SDA can report on anti-CRISPR protein inhibition of SpCas9; FIG. 27G Z-prime factor of the SDA.

FIG. 28A-28C—Generalization of the SDA to Cpf1—FIG. 28A the Cpf1/Cas12 protein/DNA complex has a different structure from Cas9 and requires optimization of the potential fluorophore attachment sites (Yamano, T et al., Cell, 2016). FIG. 28B Gel-cleavage screening of fluorophore attachments sites (NTS=non-targeting strand; TS=targeting strand) with different Cpf1 orthologs AsCpf1, LbCpf1, and FnCpf1. FnCpf1 produced a resolvable product after cleavage. FIG. 28C Fluorophore attachment to the non-target strand (NTS) yields robust quenching compared to the target strand (TS). Activity appears dependent on a TITN PAMi

FIG. 29 High Throughput Screening Pipeline for SpCas9, FnCpf1, and SaCas9 with assays, and screening to achieve lead compounds for Cas inhibitors.

FIG. 30—Screening campaigns against Cas nucleases included small molecule screening against SpCas, SaCas9 and FnCas12, with Apo nuclease used as an ‘inhibited’ positive control and DMSO used as the negative control. Scatterplots of SpCas9/gRNA complexes were introduced through either plasmid (left) or ribonucleoprotein (RNP) nucleofection (right).

FIG. 31A-31F FIG. 31A Cell viability of HEK293 cell in the presence of lead compounds. FIG. 31B Upper: Immunoblotting analysis of SpCas9 expression in the presence of CD25; Lower:immunoblotting analysis GFP expression of eGFP-disruption assay. FIG. 31C Dose-dependent inhibition in eGFP-disruption assay. FIG. 31D Flow-cytometric analysis of eGFP-disruption assay. FIG. 31E Dose-dependent inhibition in Hibit knockin assay. FIG. 31F Dose-dependent inhibition of CD25 against SpCas9 or FnCpf1.

FIG. 32—Structure Activity Relationship studies measuring normalized inhibition of CD25 analogs, pictured left.

FIG. 33A-33E SpCas9 Inhibitor Biochemical validation studies. FIG. 33A Gel cleavage analysis of CD25. FIG. 33B In vitro pulldown assay of SpCas9 by the CD24-biotin conjugate. FIG. 33C-D BLI measuring CD25 binding to SpCas9 at different concentrations FIG. 33C and versus biotin FIG. 33D. FIG. 33E STD NMR identifying potential binding atoms of CD25 bound to SpCas9.

FIG. 34—SpCas9 inhibitor increased specificity.

FIG. 35A-35C—FIG. 35A A fluorescence-based strand displacement assay (SDA) for monitoring Cas nuclease activity. Following cleavage, a fluorophore-bearing double stranded oligo (DS-fluor) is displaced by a quencher (Q)-baring displacer strand (Disp-Q), resulting in a decrease in fluorescence signal. FIG. 35B DS-fluor is not quenched in the presence of Disp-Q unless the duplex is disrupted by cleavage via an active SpCas9-gRNA complex (RNP). FIG. 35C A correct NNGRR(T) PAM-sequence is required for quenching in the SDA. Optimizing the ratio of DS-fluor to SaCas9-gRNA. Adequate fluorescence knockdown was observed at a 1:2 ratio. Optimizing the ratio of DS-fluor to Disp-Q. A Z′ factor of 0.74 was measured for the SDA in 384 well-format.

FIG. 36A-36F FIG. 36A Secondary screening on hits identified from primary screening. FIG. 36B Dose-dependent inhibition of SaCas9 by 29 top hits selected from secondary screening. FIG. 36C Chemical structures of the top 4 hits from dose-dependent eGFP-disruption assay. FIG. 36D-36F Dose-dependent inhibition by hits in HiBiT assay FIG. 36D, eGFP-autofluorescence counterscreen FIG. 36E, and T7 endonuclease assay FIG. 36F for SaCas9 in the presence of top 4 hits.

FIG. 37A-37G SaCas9 Hit validation. FIG. 37A Structure of the most active SaCas9 inhibitor, F128-0030. FIG. 37B Dose-dependent eGFP-disruption assay by F128-0030. FIG. 37C Western blot from GFP-disruption assay by F128-0030. FIG. 37D Dose-dependent DNA-gel cleavage assay, FIG. 37E HiBiT assay, FIG. 37F toxicity studies, and FIG. 37G NGS for F128-0030.

FIG. 38—Structure-Activity Relationship studies of Compounds 1-28, let with normalized % inhibition at 10 μm.

FIG. 39A-39H—FIG. 39A Specificity of SaCas9 inhibition by F128-0030 in eGFP-disruption assay. FIG. 39B Specificity of SaCas9 inhibition by F128-0030 in T7 endonuclease assay. FIG. 39C Specificity of SaCas9 inhibition by F128-0030 in western blot for eGFP disruption. FIG. 39D STD NMR for F-substituted analog of F128-0030 in presence of SaCas9. FIG. 39E Dose-dependent binding of F-substituted analog of F128-0030 in presence of SaCas9 by 19F NMR. FIG. 39F Pulldown experiment with biotinylated analog of F128-0030. FIG. 39G BLI measuring small-molecule binding with the SaCas9:gRNA complex. FIG. 39H Steady-state analysis of the BLI binding results to determine the dissociation constant.

FIG. 40A-40G—FIG. 40A Schematic of the assay. Following SpCas9 cleavage, a fluorophore bearing double stranded oligo (DS-Fluor) is displaced by a quencher (Q)-baring displacer strand (Disp-Q), resulting in a decrease in fluorescent signal. FIG. 40B DS-Fluor fluorescence is not quenched in the presence of Disp-Q unless the duplex is disrupted by cleavage via an active SpCas9:gRNA complex. A single DNA strand with fluorophore (SS-Fluor) can be completely quenched by Disp-Q in the absence of an unlabeled complementary strand. Error bars represent standard deviation from 3 technical replicates (n=3). FIG. 40C Quenching is dependent on the presence of an NGG PAM in the DS-Fluor when using SpCas9, indicating the specificity of the interaction. Error bars represent standard deviation from 3 technical replicates (n=3). FIG. 40D Inhibition of SpCas9 by two anti-CRISPR proteins, AcrIIA4 and AcrVA1, as monitored by the strand displacement assay. FIG. 40E Gel-monitored cleavage of FAM-labeled oligos (100 nM) by SpCas9 (500 nM) in a PAM-dependent manner. FIG. 40F SaCas9 activity in the strand displacement assay is dependent on an NNGRRT PAM sequence. Error bars represent standard deviation from 3 technical replicates (n=3). FIG. 40G Gel-monitored cleavage of FAM-labeled oligos (100 nM) by SaCas9 (500 nM) in a PAM-dependent manner.

FIG. 41A-41E—FIG. 41A Cas12 enzymes bind DNA in a reverse orientation compared to Cas9. Generalization of the SDA to Cas12 enzymes requires reconsideration of the fluorophore location on either the non-targeting strand (NTS) or the targeting strand (TS). FIG. 41B FnCas12 activity can be measured in the SDA using an NTS-AF647-labeled DS-Fluor substrate, and is dependent on an TTTN PAM. Error bars represent standard deviation from 3 technical replicates (n=3). FIG. 41C FnCas12 activity can also be measured in a PAM-dependent fashion using the TS-labeled DS-Fluor. The quenching yield is much lower compared to the NTS-Fluor architecture. Error bars represent standard deviation from 3 technical replicates (n=3). FIG. 41D Inhibition of FnCas12 by the type V-targeting anti-CRISPR AcrVA1, as monitored by the strand displacement assay. The type-II anti CRISPR AcrIIA4 does not inhibit FnCas12. Error bars represent standard deviation from 3 technical replicates (n=3). FIG. 41E Gel-monitored cleavage of the NTS- and TS-FAM labeled oligos (100 nM) by FnCas12 (500 nM) shows agreement with the SDA results.

FIG. 42A-42F—FIG. 42A Optimizing the substrate:quencher ratio. FIG. 42B Optimization of the relative ratio of the SpCas9:gRNA complex (1-200 nM) to DS-Fluor (fluorophore is AF647, fixed at 1 nM) at a single Disp-Q concentration (5 nM). Using a 5-fold excess of SpCas9:gRNA maximizes activity while minimizing background quenching from SpCas9 simply binding to DNA. Error bars represent standard deviation from technical replicates (n=3). FIG. 42C Optimization of the relative ratio of Disp-Q (1-200 nM) to DS-Fluor (fluorophore is AF647, fixed at 1 nM) at a single SpCas9:gRNA concentration (5 nM). Error bars represent standard deviation (n=3). FIG. 42D Time course of FIG. 42F. FIG. 42E Z score between negative control SpCas9:sgRNA and positive control SpCas9. 25 ul per well of 10 nM SpCas9:sgRNA (1:1.2) complex or 25 ul per well of 10 nM SpCas9 was distributed a 384-well plate. 25 ul of 0.5 nM FAM labeled dsDNA oligo and 2.5 nM ssDNA Quencher was transferred and incubated at 37° C. for 2.5 h before read with microplate reader. Z score between SpCas9:sgRNA (negative control) and SpCas9 (positive control) was calculated. FIG. 42F Screening result of SDA assays against 122,409 compounds. Dots in orange, green and blue represent DMSO control, Cas9 without sgRNA, and small molecules, respectively. The screen was performed in duplicate.

FIG. 43A-43B—FIG. 43A Secondary screening result of 547 hit compounds using eGFP disruption assay. Orange, green, blue dots represent DMSO control, Cas9, and small molecules respectively. The screen was performed in duplicate. FIG. 43B The bar plot of inhibition of 16 hit compounds in tertiary HiBit knockin assay. Empty box: BRD0539 as positive control, blue box: hit compounds. Core structure of hit compounds is shown.

FIG. 44A-44I Cellular characterization and validation of BRD7586 (also referred to herein as CD25). FIG. 44A Dose-dependent inhibition of SpCas9 by BRD7586 in comparison with reduced sulfur analog in eGFP disruption assay in U2OS.eGFP.PEST cells. U2OS cells transfected with SpCas9 plasmid and sgRNA plasmid were incubated with compound concentrations from 0.3 uM to 20 uM for 24 h. EC₅₀ of BRD7586 is 5.55 uM, EC₅₀ of less active analog is not available. FIG. 44B Flow-cytometric analysis of eGFP disruption assay. U2OS.eGFP.PEST cells nucleofected with SpCas9 plasmid and sgRNA plasmid were incubated with indicated amount of BRD7586 for 24 h. FIG. 44C Dose-dependent inhibition of SpCas9 by BRD7586 in comparison with reduced sulfur analog in eGFP disruption assay in U2OS.eGFP.PEST cells. U2OS cells transfected with SpCas9 plasmid and sgRNA plasmid were incubated with compound concentrations from 0.3 uM to 20 uM for 24 h. EC₅₀ of BRD7586 is 5.55 uM, EC₅₀ of less active analog is not available. FIG. 44D Dose-dependent inhibition of SpCas9 by BRD7586 in comparison with reduced sulfur analog in HiBit knockin assay in HEK293T cells. HEK293T cells transfected with SpCas9 plasmid, sgRNA plasmid and ssODN were incubated with compound concentrations from 0.06 uM to 20 uM for 24 h. EC₅₀ of BRD7586 is 5.04 uM, EC₅₀ of less active analog is 15.42 uM. FIG. 44E Dose-dependent expression in the presence of BRD7586. Upper panel: immunoblotting analysis of SpCas9 expression in U2OS cells in the presence of BRD7586. Indicated amount of BRD7586 was incubated with U2OS cells transfected with SpCas9 plasmid for 24 h. Lower panel: immunoblotting analysis of GFP expression in U2OS cells transfected with SpCas9 and sgRNA plasmids in the presence of BRD7586. U2OS.eGFP.PEST cells were transfected with SpCas9 plasmid and sgRNA plasmid, then incubated with indicated concentration of BRD7586 for 24 h. FIG. 44F Viability of U2OS.eGFP.PEST cells and HEK293T cells in the presence of BRD7586. Cells were incubated with 10-20 uM of compound for 24 h. Cell viability was determined by CellTiter-Glo to generate luminescent signal proportional to ATP presence. Error bars represent SD across three replicates (n=3). FIG. 44G Stability of BRD7586 in mouse plasma and microsome determined by ultra-performance liquid chromatography-mass spectrometry (UPLC/MS) . . . . Error bars represent SD across two replicates (n=2). FIG. 44H-FIG. 44I Dose-dependent inhibition of SpCas9 targeting EMX1(1) gene in HEK293T cells at site 1 (44H) and site 2(44I). Indicated amount of BRD7586 was incubated with HEK293T cells transfected with Speas9 plasmid and EMX1(1) sgRNA plasmid for 48 h. Genomic DNA was extracted and subjected to next-generation sequencing analysis. On-target versus off-target ratio was analyzed. P-value was calculated by unpaired two-tailed t-test.: P<0.05, **: P<0.01, ***: P<0.001. Error bars represents ±SD across three replicates (n=3).

FIG. 45A-45E Biochemical characterization and validation of BRD7586. FIG. 45A Saturation transfer difference (STD) NMR with SpCas9 and BRD7586. FIG. 45B BLI measuring BRD7586 binding with SpCas9:gRNA complex. Streptavidin sensors were loaded with uM of BRD7586, and the interaction was followed by varying SpCas9:gRNA complexes from 0.01 to 1 uM and subsequent dissociation. FIG. 45C A global 2:1 (small molecule: protein) model was used to plot the steady state and determine the binding constant. FIG. 45D In vitro pulldown experiment of SpCas9 by BRD7586-biotin conjugate. Streptavidin magnetic beads preloaded with BRD7586-biotin conjugate or biotin-azide were incubated with 0.1 uM SpCas9 for 12 h with rotation. Bound SpCas9 was eluted from streptavidin beads. BRD7586 (20 uM) was used as competitor. FIG. 45E Dose dependent inhibition of SpCas9 by BRD7586 in DNA gel cleavage.

FIG. 46A-46H Binding site studies. FIG. 46A Structure of Diazirine analog of BRD7586 (A17). FIG. 46B Structure of (A18). (A17) was crosslinked to SpCas9 peptides followed by tagging with TAMRA-azide. FIG. 46C Precursor pattern distribution (MS1) and database assignment (MS2) for SpCas9 peptide (SEQ ID NO: 62) crosslinked to the photo-A18. FIG. 46D BRD7586 docked to SpCas9, binding to the HNH nuclease and helical recognition domains. The binding pocket of BRD7586 FIG. 46E A17 (magenta) is poised to insert into residue Q807 (cyan). FIG. 46F Ligand-residue interaction map between BRD7586 and SpCas9, where N808 has been predicted to interact with oxygen as a hydrogen bond acceptor. The inactive compound (sulfide analog) is unable to interact with N808, perhaps explaining its significantly decreased activity. FIG. 46G The position of N808 in respect to H840, BRD7586 is interacting with HNH domain. FIG. 46H BRD7586 binds to the HNH nuclease domain, interacting with N808 though not H840, the residue responsible for catalyzing the cleavage of the target DNA strand. We hypothesize that BRD7586 is an allosteric inhibitor, preventing HNH nuclease from adopting the proper conformation to cleave the target strand and from allosterically activating RuvC for cleavage of the non-target strand.

FIG. 47A-47F FIG. 47A Structure of 15 hit compounds in HiBit knock-in assay. FIG. 47B Counterscreen of tertiary HiBit knock-in assay. Viability of 15 hit compounds was tested in CellTiter Glo assay in HEK293T cell. Compounds whose viability are less than 80% are removed. FIG. 47C Structure-activity relationship studies of BRD7586 in the eGFP-disruption assay and HiBit knockin assay. Orange dots represent BRD7586, A1 and A2, green dots represent reduced sulfur analogs. FIG. 47D Structure-activity relationship compounds of A1 and A2. FIG. 47E Structure-activity relationship studies of A1 in the eGFP-disruption assay. FIG. 47F Structure-activity relationship studies of A2 in the eGFP-disruption assay.

FIG. 48A-48B FIG. 48A GFP expression in the presence of Cas9 only with increasing concentration of BRD7586. FIG. 48B Viability of U2OS.eGFP.PEST cells and HEK293T cells in the presence of A1 and A2. Cells were incubated with 10-20 uM of compound for 24 h. Cell viability was determined by CellTiter-Glo to generate luminescent signal proportional to ATP presence. Error bars represent SD across three replicates (n=3).

FIG. 49A-49B FIG. 49A Structure of BRD7586-biotin conjugate and biotin-azide conjugate. FIG. 49B BLI measuring BRD7586-biotin and biotin-azide binding with SpCas9:gRNA complex.

FIG. 50 Immunoblotting analysis of photo-A18 crosslinked with SpCas9.

FIG. 51 plot of the primary screen of ˜55,000 compounds of ICCB Libraries for SaCas9.

FIG. 52 depicts eGFP assay to determine activity in cells.

FIG. 53 plot identifying compounds of interest of SaCas9.

FIG. 54 eGFP Dose Response on eGFP hits for SaCas9 compounds at 5 μM, 10 μM and 20 μM.

FIG. 55 overview of HiBiT assay.

FIG. 56 HiBiT performance of top eGFP Hits for SaCas9 compounds F128-0030, D664-0047, D226-0165, and E922-0258.

FIG. 57A depicts T7 Endonuclease Assay; FIG. 57B T7 Endonuclease assay of top compounds E922-0258, E922-1394, F128-0030, and F128-0043.

FIG. 58 SAR of several top compounds in SaCas9 assays., including % GFP disruption and % GFP positive cells for compounds E922-0258, G362-0815, E922-1394, and C429-0599 (upper panel) and compounds F128-0030, F083-0191, F128-0043, and 8012-1534 (lower panel).

FIG. 59 investigation of F128-0030 activity against SaCas9, SpCas9, FnCpf1, gel on left shows % Indel, chart on right shows % Inhibition against SaCas9, SpCas9 and FnCpf1.

FIG. 60 gel cleavage of F128-0030, at 5 μM, 10 μM, 20 μM, 30 μM, 40 μM, and 50 μM.

FIG. 61A-61B NMR spectra FIG. 61A F-NMR of VS381, active SAR of F128-0030;

FIG. 61B saturation-transfer difference (STD) NMR of VS381.

FIG. 62A-62G Development of the cumulative activity assay (CAA) FIG. 62A Schematic of the CAA. A double-stranded oligonucleotide containing a fluorophore is cleaved by SpCas9. Following cleavage, the non-fluorophore-containing strand of the oligonucleotide substrate is displaced by a quencher (Q)-bearing oligonucleotide, decreasing the fluorescence signal through fluorescence resonance energy transfer (FRET); FIG. 62B Demonstration of the SpCas9 CAA. The fluorescence of the SpCas9-specific substrate is not quenched in the presence of quencher unless the DNA duplex is cleaved via an active SpCas9:gRNA complex. A single-stranded DNA containing the fluorophore (SS-DNA) can be completely quenched in the absence of an unlabeled complementary strand. Error bars represent the mean±standard deviation (SD) from 7 independent replicates. p=3.3×10⁻¹⁰ for SpCas9:gRNA (4th bar) compared to SpCas9 only (3rd bar) (unpaired t-test, two-tailed); FIG. 62C SpCas9 CAA with varying NGG PAM sequences to demonstrate the specificity of the reaction. Light gray bars show results with SpCas9 only, and dark gray bars show results with SpCas9:gRNA complex. Error bars represent mean t SD from 8 independent replicates. For SpCas9:gRNA compared to SpCas9 only, p=3.8×10⁻¹⁸ for TGG PAM, p=3.1×10⁻⁶ for TGC PAM, and p=6.0×10⁻⁷ for ACC PAM (unpaired t-test, two-tailed); FIG. 62D Gel-monitored cleavage of FAM-labeled oligos by SpCas9:gRNA complex in a PAM-dependent manner. A representative image from 2 independent experiments is shown; FIG. 62E Inhibition of SpCas9 by two AcrIIA4 as monitored by the CAA. Error bars represent mean t SD from 3 independent replicates. p=2.2×10⁻⁵ for AcrIIA4 at 10 μM compared to buffer only (unpaired t test, two-tailed); FIG. 62F SaCas9 CAA with varying NNGRRT PAM sequences to demonstrate the specificity of the reaction. Light gray bars show results with SaCas9 only, and dark gray bars show results with SaCas9:gRNA complex. Error bars represent mean±SD from 8 independent replicates. For SaCas9:gRNA compared to SaCas9, p=3.9×10⁻²⁰ for ACGGGT PAM, p=7.1×10⁻⁴ for ACGGTI PAM, and p=8.5×10⁻⁵ for TGCCCA PAM (unpaired t-test, two-tailed); FIG. 62G Gel-monitored cleavage of FAM-labeled oligos by SaCas9:gRNA complex in a PAM-dependent manner. Image from a single experiment is shown.

FIG. 63A-63E Generalization of the CAA to different Cas systems FIG. 63A Schematic of the substrate recognition by SpCas9 and Cas12a. Cas12a enzymes bind DNA in a reverse orientation compared to Cas9. Generalization of the cumulative activity assay (CAA) to Cas12a enzymes requires optimization of the fluorophore location on either the non-targeting strand (NTS) or the targeting strand (TS); FIG. 63B FnCas12a CAA with varying PAM sequence using a NTS-labeled fluorophore. Light gray bars show results with FnCas12a only, and dark gray bars show results with FnCas12a:gRNA complex. Error bars represent mean±SD from 3 independent replicates. For FnCas12a:gRNA compared to FnCas12a, p=2.8×10⁻⁷ for TTIC PAM, p=6.4×10¹ for TIGC PAM, and p=0.15 for AAAG PAM (unpaired t-test, two-tailed);

FIG. 63C FnCas12a CAA with varying PAM sequence using a TS-labeled fluorophore. Light gray bars show results with FnCas12a only, and dark gray bars show results with FnCas12a:gRNA complex. Error bars represent mean±SD from 3 independent replicates. For FnCas12a:gRNA compared to FnCas12a, p=4.5×10⁻⁷ for TTIC PAM, p=0.030 for TTGC PAM, and p=0.19 for AAAG PAM (unpaired t-test, two-tailed); FIG. 63D Gel-monitored cleavage of the NTS- and TS-FAM labeled oligos (100 nM) by FnCas12a (500 nM). Image from a single experiment is shown;

FIG. 63E Inhibition of FnCas12a by two anti-CRISPR proteins, AcrIIA4 and AcrVA1, as monitored by the CAA. Error bars represent mean±SD from 3 independent replicates. p=5.4×10⁻⁶ for AcrVA1 at 5 μM compared to buffer only, and p=0.16 for AcrIIA4 at 5 μM compared to buffer only (unpaired t-test, two-tailed).

FIG. 64A-64I High-throughput optimization and screening with the CAA FIG. 64A Optimization of double-stranded substrate (DS-AF647) concentration relative to single-stranded substrate (SS-AF647) fully quenched by binding to Disp-Q. Error bars represent mean SD from 7 independent replicates. p=1.1×10⁻¹⁰ for SS-AF647 compared to DS-AF647 at 1 nM (unpaired t test, two-tailed); FIG. 64B Optimization of relative ratio of SpCas9-gRNA (1-20 nM) to DS-AF647 (1 nM) with fixed Disp-Q (5 nM). SS-DNA indicates SS-AF647 fully quenched by binding to Disp-Q. Error bars represent mean SD from 7 independent replicates. p=1.6×10⁻⁸ for SpCas9-gRNA compared to SpCas9 at the 1:5 ratio (unpaired t test, two-tailed); FIG. 64C Optimization of relative ratio of Disp-Q (1-20 nM) to DS-AF647 (1 nM) with fixed SpCas9-gRNA (5 nM). Error bars represent mean±SD from 7 independent replicates. p=1.5×10⁻⁹ for SpCas9-gRNA compared to SpCas9 at the 1:5 ratio (unpaired t test, two-tailed); FIG. 64D DNA cleavage over time with a varying amount of SpCas9-gRNA and fixed amount of DS-AF647 (1 nM). Error bars represent mean SD from 3 independent replicates. p=0.0128 for 150 min compared to 0 min at the 1:5 ratio (unpaired t-test, two-tailed); FIG. 64E High-throughput validation of the CAA using 10 nM SpCas9:gRNA (1:1.2) or 10 nM SpCas9 (25 μL) distributed to a 384-well plate followed by the addition of 1 nM labeled DS-AF647 and 5 nM Disp-Q (25 μL); FIG. 64F High-throughput screening against 122,409 compounds performed in duplicate. To compare results from different screening plates, Z-scores from each compound were normalized by setting the ‘DMSO control’ as 0 and ‘Cas9 without gRNA’ as 1; FIG. 64G Secondary screening of 547 hit compounds using eGFP disruption assay. The screen was performed in duplicate and normalized as in 64F. Blue dots at the right and above of the gray lines exhibit compounds with Z-scores >3 in both independent experiments; FIG. 64H Inhibition of Cas9 by hit compounds in tertiary HiBiTknock-in assay. The empty box represents the BRD0539 positive control, the colored boxes represent hit compounds, and the blue box represents the most active compound (BRD7586); FIG. 64I Structures of BRD7586 and the previously reported SpCas9 DNA-binding inhibitor, BRD0539.

FIG. 65A-65H Cellular validation of BRD7586 FIG. 65A Dose-dependent inhibition of SpCas9 by BRD7586 in the eGFP disruption assay using plasmid and RNP delivery (U2OS.eGFP.PEST, 24 h, n=6 independent replicates). For 20 μM compared to DMSO, p=2.6×10⁻⁹ (plasmid) and p=1.9×10⁻⁷ (RNP); FIG. 65B Inhibition of eGFP-targeting SpCas9 by BRD7586 in U20S.eGFP.PEST nucleofected with RNP (24 h, representative images from 6 independent experiments); FIG. 65C Dose-dependent inhibition of SpCas9 by BRD7586 in the HiBiT knock-in assay using plasmid and RNP delivery (HEK293T, 24 h, n=6 independent replicates). For 20 μM compared to DMSO, p=6.2×10⁻¹¹ (plasmid) and p=9.0×10⁻¹⁰ (RNP);

FIG. 65D Dose-dependent inhibition of eGFP-targeting SpCas9 in U2OS.eGFP.PEST as measured by deep sequencing. Cells were nucleofected with plasmids or RNP and incubated with BRD7586 (24 h, n=4 independent replicates). For 20 μM compared to DMSO, p=4.3×10⁻¹⁰ (plasmid) and p=3.7×10⁻⁴ (RNP); FIG. 65E Dose-dependent inhibition of SpCas9 targeting EMX1, FANCF, or VEGFA in HEK293T. Cells were transfected with plasmids and incubated with BRD7586 (24 h, n=6 or 7 independent replicates). For 20 pM compared to DMSO, p=3.9×10⁻¹⁰ (EMX1), p=1.7×10⁻⁸ (FANCF), and p=3.8×10⁻⁸ (VEGFA); FIG. 65F Effect of BRD7586 on specificity of SpCas9 targeting EMX1, FANCF, or VEGFA in HEK293T. Cells were transfected with plasmid and incubated with BRD7586 (48 h, n=6 or 7 independent replicates). Specificity was calculated as a ratio of normalized indel frequencies. For 20 μM compared to DMSO, p=9.1×10⁻⁶ (EMX1), p=0.0019 (FANCF), p=0.0019 for (VEGFA); FIG. 65G Immunoblotting of SpCas9 expression in HEK293T or U2OS.eGFP.PEST transfected with SpCas9 plasmid and incubated with BRD7586 (24 h). Representative images from 2 (HEK293T) or 3 (U2OS.eGFP.PEST) independent replicates are shown; FIG. 65H Viability of HEK293T cells or U2OS.eGFP.PEST incubated with BRD7586 for 24 h as measured by CellTiter-Glo. For 20 μM compared to DMSO, p=0.062 (HEK293T) and p=0.17 (U2OS.eGFP.PEST). In this figure, all the error bars represent mean SD (replicate numbers are indicated in each panel). p values were calculated from t-tests (unpaired, two-tailed).

FIG. 66A-66E Structure activity relationship studies with BRD7586 FIG. 66A Structure of the compounds used for SAR studies. The first box represents substitutions around the phenyl ring (R¹), and the second box represents variation of the thiazole endcap (R²); FIG. 66B Single change SAR compound's activity in the eGFP disruption assay as compared to BRD7586; FIG. 66C Single change SAR compound's activity in the HiBiT knock-in assay as compared to BRD7586; FIG. 66D Double change SAR compound's activity in the eGFP disruption assay as compared to BRD7586; FIG. 66E Double change SAR compound's activity in the HiBiT knock-in assay as compared to BRD7586.

FIG. 67A-67C Biochemical binding studies of BRD7586 FIG. 67A Saturation transfer difference (STD)NMR of 20 μM BRD7586 with and without 5 μM SpCas9:gRNA complex. STD-NMR was calculated through subtraction of NMR signal with and without STD magnetization signal; FIG. 67B Bio-layer interferometry (BLI) binding plot for Biotin-BRD7586 and SpCas9:gRNA complex. BLI experiment was performed using 1 μM of Biotin-BRD7586 on streptavidin sensors followed by association with different concentrations of SpCas9:gRNA complex and subsequent dissociation; FIG. 67C Steady-state analysis of the BLI binding results to determine the dissociation constant. A global model was used to plot the steady state and determine the binding constant.

FIG. 68A-68E Mechanism of action studies of BRD7586 FIG. 68A Chemical structures of Diazirine-BRD7586 and an inactive analog BRD0033. Substitutions from parent compound (BRD7586) are labeled in blue (Diazirine-BRD7586) and red (BRD0033); FIG. 68B Engagement of BRD7586 to purified SpCas9. Diazirine-BRD7586 (1 μM) was photo-crosslinked to SpCas9:gRNA (1 μM) and was tagged with TAMRA-azide through click chemistry. TAMRA fluorescence was detected only in the presence of UV, click chemistry reagents, and Diazirine-BRD7586. A competition with BRD7586 (5 μM) resulted in decreased photo-crosslinking. Representative images from 2 independent experiments are shown; FIG. 68C Target engagement of BRD7586 in live cells. HEK293T cells transiently expressing Cas9 were treated with Diazirine-BRD7586 followed by in-cell photo-crosslinking. Cells lysis and click chemistry tagged the Diazirine-BRD7586-bound proteins with biotin, which were pulled down by streptavidin beads and probed for the presence of SpCas9 by immunoblotting. Representative images from 2 independent experiments are shown; FIG. 68D Activity of BRD0033 and BRD7586 in the eGFP disruption assay. Error Bars represent mean±SD from 6 independent replicates. p=0.27 for BRD0033 at 20 μM compared to DMSO, and p=3.2×10⁻⁹ for BRD7586 at 20 μM compared to DMSO, (unpaired t-test, two-tailed); FIG. 68E Activity of BRD0033 and BRD7586 in the HiBiT knock-in assay. Error Bars represent mean±SD from 6 independent replicates. p=0.047 for BRD0033 at 20 μM compared to DMSO, and p=4.5×10⁻⁹ for BRD7586 at 20 μM compared to DMSO (unpaired t-test, two-tailed).

FIG. 69A-69E Cumulative activity assay (CAA) validation FIG. 69A Schematic of the differently labeled PAM fluorescence polarization substrates. FIG. 69B Fluorescence polarization assay comparing 0-PAM and 12-PAM substrates with SaCas9 at multiple concentrations of unlabeled ligand. Substrates were labeled with FAM on the 3′ end. SaCas9 showed specificity for the 12-PAM substrate that decreased with increasing amounts of unlabeled competitor. Error bars represent SD from 3 independent experiments. For ‘0× UL’ compared to ‘No SaCas9’, p>0.05 with the 0-PAM DNA and p≤0.001 with the 12-PAM DNA (unpaired t test, two-tailed). FIG. 69C Fluorescence polarization assay comparing 0-PAM and 12-PAM substrates with FnCas12a at multiple concentrations of unlabeled ligand. Substrates were labeled with FAM on the 3′ end. FnCas12a showed no specificity dependent upon the presence of PAM-binding sites. Error bars represent SD from 3 independent experiments. For ‘0× UL’ compared to ‘No FnCas12a’, p≤0.0001 both with the 0-PAM and 12-PAM DNA (unpaired t test, two-tailed).

FIG. 69D Fluorescence polarization assay comparing 0-PAM and 12-PAM substrates with FnCas12a at multiple concentrations of unlabeled ligand. Substrates were labeled with FAM on the 5′ end. FnCas12a showed no specificity dependent upon the presence of PAM-binding sites. Error bars represent SD from 3 independent experiments. For ‘0× UL’ compared to ‘No FnCas12a’, p≤0.001 with the 0-PAM DNA and p≤0.0001 with the 12-PAM DNA (unpaired t test, two-tailed). FIG. 69E Inhibition of SpCas9 by AcrIIA11 monitored by the CAA. Error bars represent SD from 4 independent experiments. p≤0.0001 for AcrIIA11 at 10 μM compared to buffer only (unpaired t test, two-tailed). FIG. 69F The fluorescence of the SaCas9-specific substrate is not quenched in the presence of quencher unless the duplex is disrupted by cleavage via an active SaCas9:gRNA complex. A single DNA strand containing the fluorophore (SS-DNA) can be completely quenched in the absence of an unlabeled complementary strand. Error bars represent SD from 4 independent experiments. p≤0.0001 for SaCas9:gRNA (4^{th} bar) compared to SaCas9 only (3rd bar) (unpaired t test, two-tailed).

FIG. 70A-70E Generalization of the cumulative activity assay FIG. 70A Demonstration of FnCas12a CAA. The fluorescence of the FnCas12a-specific substrate labeled on the non-targeting strand (NTS) is not quenched in the presence of quencher unless the duplex is disrupted by cleavage via an active FnCas12a:gRNA complex. A single DNA strand containing the fluorophore (SS-DNA) can be completely quenched in the absence of an unlabeled complementary strand. Error bars represent SD from 4 independent experiments. p≤0.0001 for FnCasd12a:gRNA (4^{th} bar) compared to FnCas12a only (3rd bar)(unpaired t test, two-tailed). FIG. 70B Demonstration of FnCas12a CAA. The fluorescence of the FnCas12a-specific substrate labeled on the targeting (TS) strand is quenched poorly even in the presence of active FnCas12a:gRNA complex. Error bars represent SD from 4 independent experiments. p≤0.0001 for FnCasd12a:gRNA (4^{th} bar) compared to FnCas12a only (3rd bar) (unpaired t test, two-tailed) FIG. 70C Gel-monitored cleavage of FAM-labeled oligos (20 nM) by Ascas12a (100 nM), LbCas12a (100 nM), and FnCas12a (100 nM) in a PAM-dependent manner.

FIG. 71A-71E Optimization of the cumulative activity assay for high-throughput screening FIG. 71A Hit compounds (Z score>3σ) identified in both the primary screen and secondary screens. Compounds were compared to BRD0539, a compound previously identified as an SpCas9 DNA binding inhibitor FIG. 71B Dose-dependent inhibition of SpCas9 by BRD7586 in the CAA. Error bars represent SD from 3 independent experiments. p≤0.05 for BRD7586 at 30 μM compared to DMSO (unpaired t test, two-tailed) FIG. 71C,D Gel-based dose-dependent inhibition (C) and quantification of inhibition (D) of SpCas9 by BRD7586 in DNA cleavage assay. gel images and error bars represent SD from 4 biological replicates. p≤0.0001 for 40 μM (6th bar) compared to 0 pM only (1st bar) (unpaired t test, two-tailed).

FIG. 72A-72E Validation of BRD7586 in cells FIG. 72A T7E1 assay for detecting indels at the eGFP gene. U20S.eGFP.PEST cells were nucleofected with plasmid and incubated with BRD7586 for 24 h. Blue arrowheads indicate uncleaved DNA and black arrowheads indicate cleaved DNA. FIG. 72B T7E1 for detecting indels at the eGFP gene. U20S.eGFP.PEST cells were nucleofected with RNP and incubated with BRD7586 for 24 h. Blue arrowheads indicate uncleaved DNA and black arrowheads indicate cleaved DNA FIG. 72C Inhibition of SpCas9 by BRD0539 and BRD7586 in the eGFP disruption assay using plasmid delivery method and RNP delivery method (U20S.eGFP.PEST cells, 24 h). Error bars represent SD from 3 or 4 independent experiments. For the plasmid-based assay, p≤0.0001 for BRD0539 and BRD7586 at 15 μM compared to DMSO (unpaired t test, two-tailed). For the RNP-based assay, p≤0.001 for BRD0539 at 15 μM compared to DMSO, and p≤0.0001 for BRD7586 at 15 μM compared to DMSO (unpaired t test, two-tailed) FIG. 72D Inhibition of SpCas9 by BRD0539 and BRD7568 in the HiBiT knock-in assay using plasmid delivery method and RNP delivery method (HEK293T cells, 48 h). Error bars represent SD from 2 or 3 independent experiments. For the plasmid-based assay, p≤0.001 for BRD0539 at 15 μM compared to DMSO, and p≤0.0001 for BRD7586 at 15 μM compared to DMSO (unpaired t test, two-tailed)

FIG. 73A-73E Counter assays to validate BRD7586 in cells FIG. 73A,B Immunoblotting analysis of SpCas9 expression in (A) HEK293T cells or (B) U20S.eGFP.PEST cells transfected with SpCas9 plasmid were incubated with BRD7586 for 24 h (n=2 or 3 independent experiments). p>0.05 for BRD7586 at 20 pM compared to DMSO in U20S.eGFP.PEST cells (paired t test, two-tailed) FIG. 73C Changes in the fluorescence intensity in the presence of BRD7586 in U20S.eGFP.PEST. Cells were treated with the compound for 24 h, and the fluorescence was measured to calculate the fraction of GFP-positive populations. Means from 3 independent experiments are shown. Due to the low SD, error bars cannot be shown. p≤0.05 for BRD7586 at 20 μM compared to DMSO (unpaired t test, two-tailed) FIG. 73D Dose-dependent inhibition of SpCas9 or LbCas12a by BRD7586 in the eGFP disruption assay using RNP delivery methods (U20S.eGFP.PEST cells, 24 h). Error bars represent SD from 3 independent experiments. For SpCas9, p≤0.01 for BRD7586 at 15 μM compared to DMSO. For LbCas12a, p>0.05 for BRD7586 at 15 μM compared to DMSO in all experiments (unpaired t test, two-tailed).

FIG. 74A-74E Biochemical validation FIG. 74A Structure of Biotin-BRD7586 and Biotin-PEG3-Azide control FIG. 74B Control BLI binding plot for Biotin-PEG3-azide and SpCas9:gRNA complex. BLI experiment was performed using 1 μM of Biotin-PEG3-azide on streptavidin sensors followed by association with different concentrations of SpCas9:gRNA complex and subsequent dissociation. BLI signal of biotin-BRD7586 and 1 μM SpCas9:gRNA complex is marked in red for comparison.

FIG. 75A-75E Mechanism of action studies of BRD7586 FIG. 75A Workflow of the chemoproteomics experiments using the diazirine-based photo-crosslinking probe to identify binding sites of BRD7586 on SpCas9 FIG. 75B T7E1 assay for measuring the activity of Diazirine-BRD7586. U20S.eGFP.PEST cells were nucleofected with Cas9 plasmid and eGFP-targeting plasmid, and the cells were incubated with the compounds for 24 h. Blue arrowheads indicate uncleaved DNA while black arrowheads indicate cleaved DNA from the T7E1 reaction FIG. 75C Chemical structure of the acid-cleavable and isotope-coded biotin-azide used for chemoproteomics experiments FIG. 75D Structure of the peptide-compound conjugates to be detected from the mass spectrometry. Note the 3:1 ratio of the isotope tag that allows reliable identification of the conjugates FIG. 75E,F Examples of the mass spectra obtained from the chemoproteomics experiments. For the comprehensive list of identified peptides (SEQ ID NO: 62), see Table 14. The isotope patterns are shown as green labels in MS1 spectra. The probe-conjugated residues are shown as green labels in MS2 spectra FIG. 75G Proposed binding pocket of BRD7586 on SpCas9. BRD7586 was docked to SpCas9 at the HNH-nuclease and helical recognition domains. (SEQ ID NO: 62).

FIG. 76A-76E Mechanism of action studies of BRD7586 FIG. 76A Structure of BRD7586 and F2537-0908 FIG. 76B Activity of BRD7586 and F2537-0908 in the eGFP disruption assay. Results from 2 independent experiments are shown FIG. 76C Activity of BRD7586 and F2537-0908 in the HiBiT knock-in assay. Results from 2 independent replicates are shown FIG. 76D Fluorescence polarization assay to detect Cas9-DNA interactions. f-DNA indicates FITC-labeled, SpCas9 PAM-containing DNA. Error bars represent SD from 3 independent replicates. p≤0.01 for BRD0539 at 5 μM compared to DMSO (unpaired t test, two-tailed)

FIG. 77 1H NMR spectrum of BRD7586.

FIG. 78 13C NMR spectrum of BRD7586.

FIG. 79 1H NMR spectrum of BRD0033.

FIG. 80 13C NMR spectrum of BRD0033.

FIG. 81 1H NMR spectrum of 3-((4-methoxyphenyl)sulfonyl)-N-(4-(pyridin-4-yl) thiazol-2-yl)propanamide (4).

FIG. 82 13C NMR spectrum of 3-((4-methoxyphenyl)sulfonyl)-N-(4-(pyridin-4-yl) thiazol-2-yl)propanamide (4).

FIG. 83 1H NMR spectrum of 3-((4-hydroxyphenyl)sulfonyl)-N-(4-(pyridin-4-yl)thiazol-2-yl)propanamide (5).

FIG. 84 13C NMR spectrum of 3-((4-hydroxyphenyl)sulfonyl)-N-(4-(pyridin-4-yl)thiazol-2-yl)propanamide (5).

FIG. 85 1H NMR spectrum of tert-butyl (2-(4-((3-oxo-3-((4-(pyridin-4-yl)thiazol-2-yl)amino)propyl)sulfonyl)phenoxy)ethyl)carbamate (6).

FIG. 86 13C NMR spectrum of tert-butyl (2-(4-((3-oxo-3-((4-(pyridin-4-yl)thiazol-2-yl)amino)propyl)sulfonyl)phenoxy)ethyl)carbamate (6).

FIG. 87 1H NMR spectrum of Biotin-BRD7586.

FIG. 88 13C NMR spectrum of Biotin-BRD7586.

FIG. 89 1H NMR spectrum of BRD7586-diazirine.

FIG. 90 13C NMR spectrum of BRD7586-diazirine.

FIG. 91 LCMS-data of SAR compounds.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995)(M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofkcer and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Non-limiting examples of optional substituents as referred to herein include halogen, alkyl, aralkyl, heteroalkyl, alkenyl, alkynyl, cycloalkyl, hydroxyl, alkoxy, amino, amido, nitro, cyano, amido, carbonyl, carboxyl, silyl, ether, alkylthio, sulfonyl, sulfonamido, ketone, aldehyde, ester, heterocyclyl, aryl, and heteroaryl.

A “substituted” hydrocarbon may have as a substituent one or more hydrocarbon radicals, substituted hydrocarbon radicals, or may comprise one or more heteroatoms.

Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (F, Cl, Br, I, etc.), boron, silicon, etc. In some embodiments, heteroatoms will be selected from the group consisting of oxygen, nitrogen, sulfur, phosphorous, and halogen (F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In some embodiments, any hydrocarbon or substituted hydrocarbon disclosed herein may be substituted with one or more substituents X, where X is independently selected at each occurrence from one or more (e.g., 1-20) heteroatoms or one or more (e.g., 1-10) heteroatom-containing groups, where, for example, X may be selected from —F; —Cl; —Br, —I; —OH; —OR*; —NH₂; —NHR*; —N(R*)₂; —N(R*)₃⁺; —N(R*)—OH; —N(—>O)(R*)₂; —O—N(R*)₂; —N(R*)—O—R*; —N(R*)—N(R*)₂; —C═N—R*; —N═C(R*)₂; —C═N—N(R*)₂; —C(═NR*)(—N(R*)₂); —C(H)(═N—OH); —SH; —SR*; —CN; —NC; —C(═O)—R*; —CHO; —CO₂H; —CO₂—; —CO₂R*; —C(═O)—S—R*; —O—(C═O)—H; —O—(C═O)—R*; —S—C(═O)—R*; —(C═O)—NH₂; —C(═O)—N(R*)₂; —NH—(C═O)—R*; —NH—(C═O)—R*; —N(R*)—C(═O)—R*; —C(═O)—NHNH₂; —O—C(═O)—NHNH₂; —C(═S)—NH₂; —(C═S)—N(R*)₂; —N(R*)—CHO; —N(R*)—C(═O)—R*; —C(═NR*)—O—R*; —O—C(═NR*)—R*; —SCN; —NCS; —NSO; —SSR*; —N(R*)—C(═O)—N(R*)₂; —N(R*)—C(═S)—N(R*)₂; —S(═O)ₙ—R*; —O—S(═O)₂—R*; —S(═O)₂-OR*; —N(R*)—S(═O)₂—R*; —S(═O)₂—N(R*)₂; —O—SO₃; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C2H₄); —Si(R*)₃; —CF₃; —O—CF₃; —O—CH₃; —O—(CH₂)₁₋₆CH₃; —PR*₂; —O—P(═O)(OR*)₂; and —P(═O)(OR*)₂; where, independently at each occurrence, R* may be H or a C₁₋₁₀ or C₁₋₈ or C₁₋₆ or C₁₋₄ hydrocarbon, including without limitation alkyl, alkenyl, alkynyl, aryl (e.g., phenyl), alkyl-aryl (e.g., benzyl), aryl-alkyl (e.g., toluyl), etc. In other embodiments, X may comprise a C₁-C₈ or C₁-C₆ or C₂-C₄ perfluoroalkyl. In other embodiments, X may a C₁-C₅ or C₂-C₆ or C₃-C₅ heterocycle (e.g., heteroaryl radical). The term “halo” or “halogen” refers to any radical of fluorine, chlorine, bromine, or iodine. In some embodiments, X is independently selected at each occurrence from —OH, —SH, —NH₂; —N(R*)₂; —F, and —Cl.

In addition, the phrase “substituted with a[n],” as used herein, means the specified group may be substituted with one or more of any or all of the named substituents. For example, where a group, such as an alkyl or heteroaryl group, is “substituted with an unsubstituted C1-C20 alkyl, or unsubstituted 2 to 20 membered heteroalkyl,” the group may contain one or more unsubstituted C1-C20 alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substituents. Examples of alkyl groups include without limitation methyl, ethyl, n-propyl, isopropyl, and tert-butyl.

As used herein, the term “straight chain Cₙ₋ₘ alkylene,” employed alone or in combination with other terms, refers to a non-branched divalent alkyl linking group having n tom carbon atoms. Any atom can be optionally substituted, e.g., by one or more substituents. Examples include methylene (i.e., —CH₂—).

The term “haloalkyl” refers to an alkyl group, in which at least one hydrogen atom is replaced by halo. In some embodiments, more than one hydrogen atom (e.g., 2, 3, 4, 5, 6, 7, 8, 9, 10, 11, 12, 13, or 14) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

As referred to herein, the term “alkoxy” refers to a group of formula —O(alkyl).

Alkoxy can be, for example, methoxy (—OCH₃), ethoxy, propoxy, isopropoxy, butoxy, iso-butoxy, sec-butoxy, pentoxy, 2-pentoxy, 3-pentoxy, or hexyloxy. Likewise, the term “thioalkoxy” refers to a group of formula —S(alkyl). Finally, the terms “haloalkoxy” and “halothioalkoxy” refer to —O(haloalkyl) and —S(haloalkyl), respectively. The term “sulfhydryl” refers to —SH. As used herein, the term “hydroxyl,” employed alone or in combination with other terms, refers to a group of formula —OH.

The term “aralkyl” refers to an alkyl moiety in which an alkyl hydrogen atom is replaced by an aryl group. One of the carbons of the alkyl moiety serves as the point of attachment of the aralkyl group to another moiety. Any ring or chain atom can be optionally substituted e.g., by one or more substituents. Non-limiting examples of “aralkyl” include benzyl, 2-phenylethyl, and 3-phenylpropyl groups.

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent.

The term “alkynyl” refers to a straight or branched hydrocarbon chain containing the indicated number of carbon atoms and having one or more carbon-carbon triple bonds.

Alkynyl groups can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “heterocycyl” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. The heteroatom or ring carbon can be the point of attachment of the heterocyclyl substituent to another moiety. Any atom can be optionally substituted, e.g., by one or more substituents. Heterocyclyl groups can include, e.g., tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected Ra” would include (but not be limited to) tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from 0, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include, e.g., dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. Cycloalkenyl moieties can include, e.g., cyclohexenyl, cyclohexadienyl, or norbornenyl.

As used herein, the term “cycloalkylene” refers to a divalent monocyclic cycloalkyl group having the indicated number of ring atoms.

As used herein, the term “heterocycloalkylene” refers to a divalent monocyclic heterocyclyl group having the indicated number of ring atoms.

The term “aryl” refers to an aromatic monocyclic, bicyclic (2 fused rings), or tricyclic (3 fused rings), or polycyclic (>3 fused rings) hydrocarbon ring system One or more ring atoms can be optionally substituted, e.g., by one or more substituents. Aryl moieties include, e.g., phenyl and naphthyl.

The term “heteroaryl” refers to an aromatic monocyclic, bicyclic (2 fused rings), tricyclic (3 fused rings), or polycyclic (>3 fused rings) hydrocarbon groups having one or more heteroatom ring atoms independently selected from 0, N (it is understood that one or two additional groups may be present to complete the nitrogen valence and/or form a salt), or S. One or more ring atoms can be optionally substituted, e.g., by one or more substituents. Examples of heteroaryl groups include, but are not limited to, ²H-pyrrolyl, ³H-indolyl, ⁴H-quinolizinyl, acridinyl, benzo[b]thienyl, benzothiazolyl, P-carbolinyl, carbazolyl, coumarinyl, chromenyl, cinnolinyl, dibenzo[b,d]furanyl, furazanyl, furyl, imidazolyl, imidizolyl, indazolyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, and xanthenyl.

The terms “arylcycloalkyl” and “arylheterocyclyl” refer to bicyclic, tricyclic, or other polycyclic ring systems that include an aryl ring fused to a cycloalkyl and heterocyclyl, respectively. Similarly, the terms “heteroarylheterocyclyl,” and “heteroarylcycloalkyl” refer to bicyclic, tricyclic, or other polycyclic ring systems that include a heteroaryl ring fused to a heterocyclyl and cycloalkyl, respectively. Any atom can be substituted, e.g., by one or more substituents. For example, arylcycloalkyl can include indanyl; arylheterocyclyl can include 2,3-dihydrobenzofuryl, 1,2,3,4-tetrahydroisoquinolyl, and 2,2-dimethylchromanyl.

The term “vicinal” refers to the configuration in which any two atoms or groups are, respectively, bonded to two adjacent atoms (i.e., the two atoms are directly bonded to one another). The term “geminal” describes a configuration in which any atoms or two functional groups are bonded to the same atom As used herein, when any two groups are said to together form a ring, unless otherwise indicated, it is meant that a bond is formed between each of said two groups, with the valences of the atoms appropriately adjusted to accommodate at least a bond (e.g., a hydrogen atom may be removed from each group).

The descriptors “C═O” or “C(O)” or “carbonyl” refers to a carbon atom that is doubly bonded to an oxygen atom “Alkyl carbonyl” has a common formula of R—C(O)— wherein R may be C1-12 alkyl, C2-12 alkenyl, C2-12 alkynyl, C3-12 cycloalkyl, C6-12 aryl, C3-12 heteroaryl, or C3-12 heterocyclyl.

The term “oxo” refers to double bonded oxygen which can be a substituent on carbon or other atoms. When oxo is a substituent on nitrogen or sulfur, it is understood that the resultant groups have the structures N—>O⁻ and S(O) and SO₂, respectively.

As used herein, the term “cyano,” employed alone or in combination with other terms, refers to a group of formula —CN, wherein the carbon and nitrogen atoms are bound together by a triple bond. The term “azide” refers to a group of formula —N₃. The term “nitro” refers to a group of formula —NO₂. The term “amine” includes primary (—NH₂), secondary (—NHR), tertiary (—NRR′), and quaternary (—N⁺RR′R″) amine having one, two or three independently selected substituents such as straight chain or branched chain alkyl, alkenyl, alkynyl, cycloalkyl, aryl, heteroaryl, heterocycle, and the like.

When any variable (e.g., R¹) occurs more than one time in any constituent or formula for a compound, its definition at each occurrence is independent of its definition at every other occurrence. Thus, for example, if a group is shown to be substituted with one or more R¹ moieties, then R¹ at each occurrence is selected independently from the Markush group recited for R¹. Also, combinations of substituents and/or variables are permissible, but only if such combinations result in stable compounds within a designated atom's normal valency.

As used herein, “unsaturated” refers to compounds or structures having at least one degree of unsaturation (e.g., at least one double or triple bond).

The term “pharmaceutically acceptable salt” refers to those salts that are within the scope of proper medicinal assessment, suitable for use in contact with human tissues and organs and those of lower animals, without undue toxicity, irritation, allergic response or similar and are consistent with a reasonable benefit/risk ratio. In some embodiments, pharmaceutically acceptable salts can be formed by the reaction of a disclosed compound with an equimolar or excess amount of acid. Alternatively, hemi-salts can be formed by the reaction of a compound with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., about one hour to about ten days and can be isolated by filtration or other conventional methods.

In some aspects, the compound is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

Geometric isomers can be represented by the symbol—which denotes a bond that can be a single, double or triple bond as described herein. Provided herein are various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

The term “enantiomers” refers to a pair of stereoisomers that are non-superimposable mirror images of each other. An atom having an asymmetric set of substituents can give rise to an enantiomer. A mixture of a pair of enantiomers in any proportion can be known as a “racemic” mixture. The term “(±)” is used to designate a racemic mixture where appropriate.

“Diastereoisomers” are stereoisomers that have at least two asymmetric atoms, but which are not mirror-images of each other. The absolute stereochemistry is specified according to the Cahn-Ingold-Prelog R—S system When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. In some chemical structures, stereocenters may be identified with “wavy” bonds indicating that the stereocenter may be in the R or S configuration, unless otherwise specified. However, stereocenters without a wavy bond (i.e., a “straight” bond) may also be in the (R) or (S) configuration, unless otherwise specified. Compositions comprising compounds may comprise stereocenters which each may independently be in the (R) configuration, the (S) configuration, or racemic mixtures.

Optically active (R)- and (S)-isomers can be prepared, for example, using chiral synthons or chiral reagents, or resolved using conventional techniques. Enantiomers can be isolated from racemic mixtures by any method known to those skilled in the art, including chiral high pressure liquid chromatography (HPLC), the formation and crystallization of chiral salts, or prepared by asymmetric syntheses.

Optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization, or sublimation, and then hydrolyzed to deliver the enantiomerically enriched compound.

Optically active compounds can also be obtained by using active starting materials. In some embodiments, these isomers can be in the form of a free acid, a free base, an ester, or a salt.

In certain embodiments, a disclosed compound can be a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by an acid or base or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides compositions and methods for inhibiting the activity of RNA-guided nucleases, methods of use therefore (e.g., inhibition or prevention of genome editing by the RNA-guided nuclease), and methods of identifying inhibitors of RNA-guided nucleases. In some examples, the RNA-guided nucleases may be RNA-guided endonucleases (e.g., Type II, Type V, or Type VI). The compositions and methods herein are based, at least in part, on the discovery of small molecule inhibitors of RNA-guided endonucleases. Methods involving small molecule inhibitors of RNA guided endonucleases are useful for the modulation of RNA-guided endonuclease activity, including rapid, reversible, dosage, and/or temporal control of RNA-guided endonuclease technologies. Methods of inhibiting activity of an RNA-guided endonuclease comprise contacting the RNA-guided endonuclease with a compound disclosed herein. In some embodiments, the compound inhibits the activity of an RNA-guided endonuclease reversibly. For example, the inhibitor compound can join (e.g., non-covalent binding) the RNA-guided endonuclease and then separate. Reversibility can be modified by varying the inhibitor composition (e.g., the addition and/or subtraction of a chemical group) or varying the environment of the interaction (e.g., changing temperature and/or pH). In some embodiments, the method is performed in vitro. In some embodiments, the method is performed in a cell. In some embodiments, the cell is a germline cell. In some embodiments, the cell is a prokaryotic cell. In some embodiments, the prokaryotic cell is a bacterium. In some embodiments, the cell is a eukaryotic cell. In some embodiments, the eukaryotic cell is a human cell, a mammalian cell, an insect cell, a plant cell, or a yeast cell. In some embodiments, the cell is in an organism. In some embodiments, the organism is a human, mammal, vertebrate, invertebrate, insect, or plant. In some embodiments, the RNA-guided endonuclease may be a Type II, a Type V, or Type VI Cas. In some embodiments, the RNA-guided endonuclease is SaCas9 or variants thereof. In some embodiments, the Cas protein is a Cas12a protein. In particular embodiments, the protein is a FnCas12a.

In some embodiments disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some embodiments disclosed herein are inhibitors of Cas12, e.g., naturally occurring Cas12 in FnCas12a, or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

### Compounds

The disclosed compounds may be in free base form unassociated with other ions or molecules. In some cases, the compounds may be pharmaceutically acceptable salts, solvates, or prodrugs thereof. One aspect provides a disclosed compound or a pharmaceutically acceptable salt. One aspect provides a disclosed compound or a pharmaceutically acceptable salt or solvate thereof. One aspect provides a solvate of a disclosed compound. One aspect provides a hydrate of a disclosed compound.

In some embodiments, the inhibitor is selected from a compound in one of Tables 1-6.

In certain embodiments, the inhibitor is an SpCas9 inhibitor and is selected from a compound in Table 2A-3B.

In embodiments, the inhibitor is an SpCas9 inhibitor and is selected from the compound according to the formula:

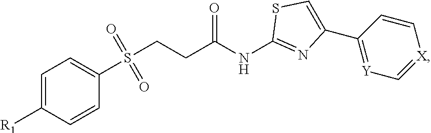

wherein X and Y are independently selected from N, and R₁ is independently selected from substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen.

In some embodiments, the compound is selected from

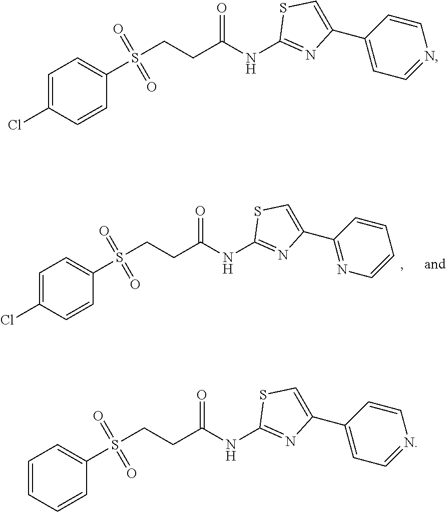 [SMILES: O=C(CCS(=O)(=O)c1ccc(Cl)cc1)Nc1nc(-c2ccncc2)cs1 ; O=C(CCS(=O)(=O)c1ccc(Cl)cc1)Nc1nc(-c2ccccn2)cs1 ; O=C(CCS(=O)(=O)c1ccccc1)Nc1nc(-c2ccncc2)cs1]

In certain embodiments, the compound is according to the formula:

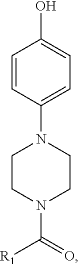

wherein R₁ is selected from

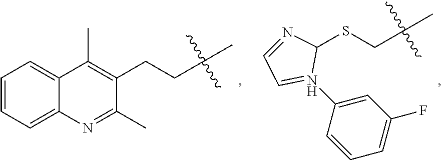

or

In certain embodiments, the inhibitor is selected from:

Table 1, 4A, 4B, or 5. In certain embodiments, the inhibitor is an SaCas9 inhibitor and is selected from a compound in Table 1.

TABLE 1
SaCas9 Inhibitors
SaCas9 Inhibitors - Smile

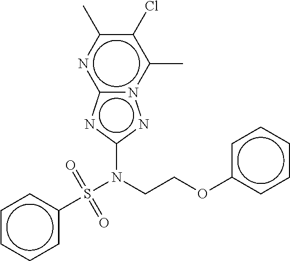

c(N(CCOc1ccccc1)S)c2CCCCC2)(═O)═O)(nn3crnc(c(Cl)c3C)C)n4

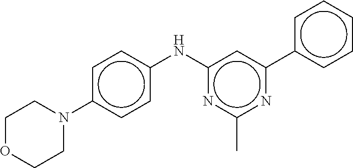

c1(Nc(cc2)ccc2N(CC3)CCO3)cc(c4ccccc4)nc(C)n1

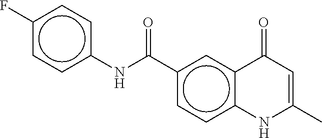

c12c(ccc(c1)c(═O)Nc(cc3)ccc3F)NC(═CC2═O)C

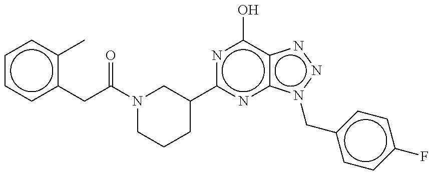

c12c(c(nc(C(CCCN3C(═O)Cc4ccccc4C)C3)n1)O)nnn2Cc(cc5)ccc5F

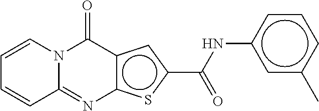

c1(C2′O)c(sc(C(═O)Nc3cccc(C)c3)c1)N═C(C═CC═C4)N24

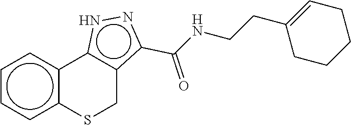

c1(CSc(c23)cccc2)c3[nH]nc1C(NCCC4═CCCCC4)═O

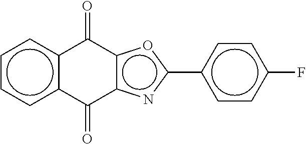

Fc1ccc(cc1)c1nc2C(═O)c3ccccc3C(═O)c2o1

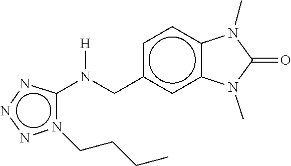

C1(═O)N(C)c2c(cc(cc2)CN([H])c3nnnn3CCCC)N1C

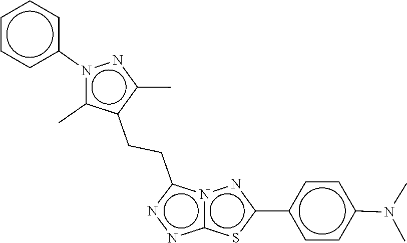

n1(c(CCc(c(C)nn2c3ccccc3)c2C)nn4)c4sc(c(cc5)ccc5N(C)C)n1

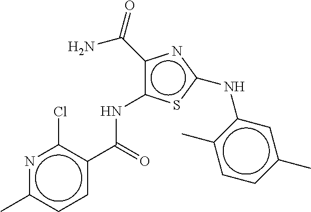

c1(NC(═O)c2ccc(nc2Cl)C)sc(nc1C(N)═O)Nc3cc(C)ccc3C

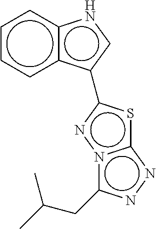

n1(n2)c(nnc1CC(C)C)sc2c3c4c([nH]c3)cccc4

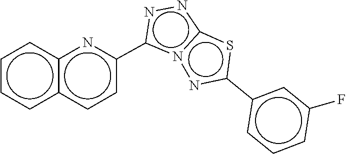

n12c(nnc1c3ccc(c4n3)cccc4)sc(c5cccc(F)c5)n2

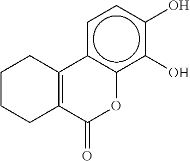

c12c(ccc(O)c1O)C3═C(C(═O)O2)cccc3

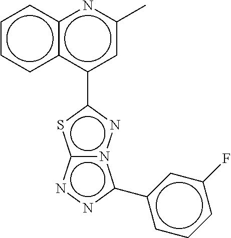

n12c(nnc1c3cccc(F)c3)sc(c(cc4C)cSc(n4)cccc5)n2

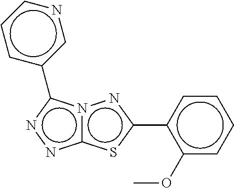

n12c(nnc1c(cccn3)c3)sc(c4ccccc4OC)n2

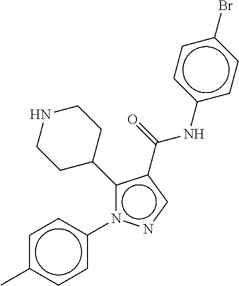

c(cnn1c2ccc(cc2)C)(C(═O)Nc(cc3)ccc3Br)c1C(CC4)CCN4

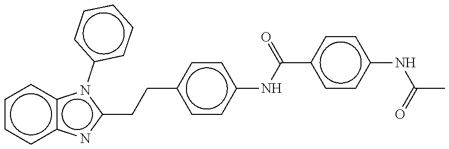

c1(CCc(cc2)ccc2NC(═O)c3ccc(cc3)NC(C)═O)nc(c4n1c5ccccc5)cccc4

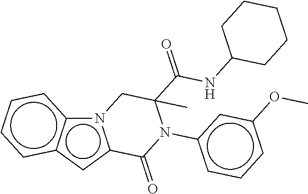

C1(C)Cn(c2c(c3)cccc2)c3C(═O)N1c4cccc(OC)c4)C(═O)NC5CCCCC5

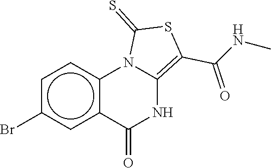

N1(c2c(cc(Br)cc2)C(N3)═O)C3═C(SC1═S)C(NC)═O

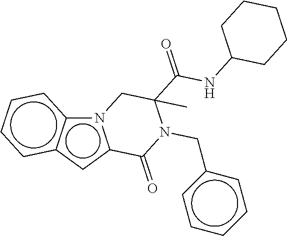

N(Cc1ccccc1)(C2═O)C(C)(Cn(c3c(cccc3)c4)c24)C(═O)NC5CCCCC5

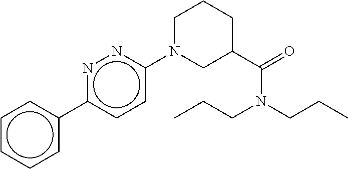

C(CCCN1c2ccc(nn2)c3ccccc3)(C1)(C(═O)N(CCC)CCC

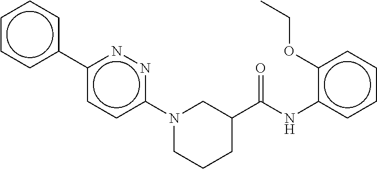

N(CCCC1C(═O)Nc2ccccc2OCC)(C1)c3ccc(nn3)c4ccccc4

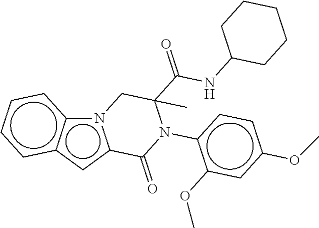

N1(c2ccc(cc2OC)OC)C(═O)c3n(CC1(C)C(═O)NC4CCCCC4)c5c(cccc5)
c3

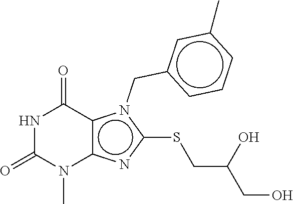

c12c(C(NC(═O)N1C)═O)n(c(SCC(CO)O)n2)Cc3cccc(C)c3

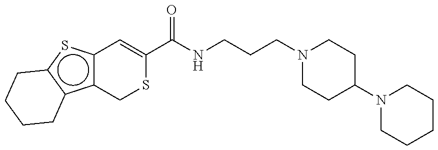

c12c(CSC(C(NCCCN(CC3)CCC3N4CCCCC4)═O)═C1)c5c(CCCC5)s2

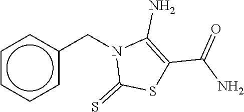

N1(Cc2ccccc2)C(═S)SC(C(N)═O)═C1N

In embodiments, the inhibitor is selected from:

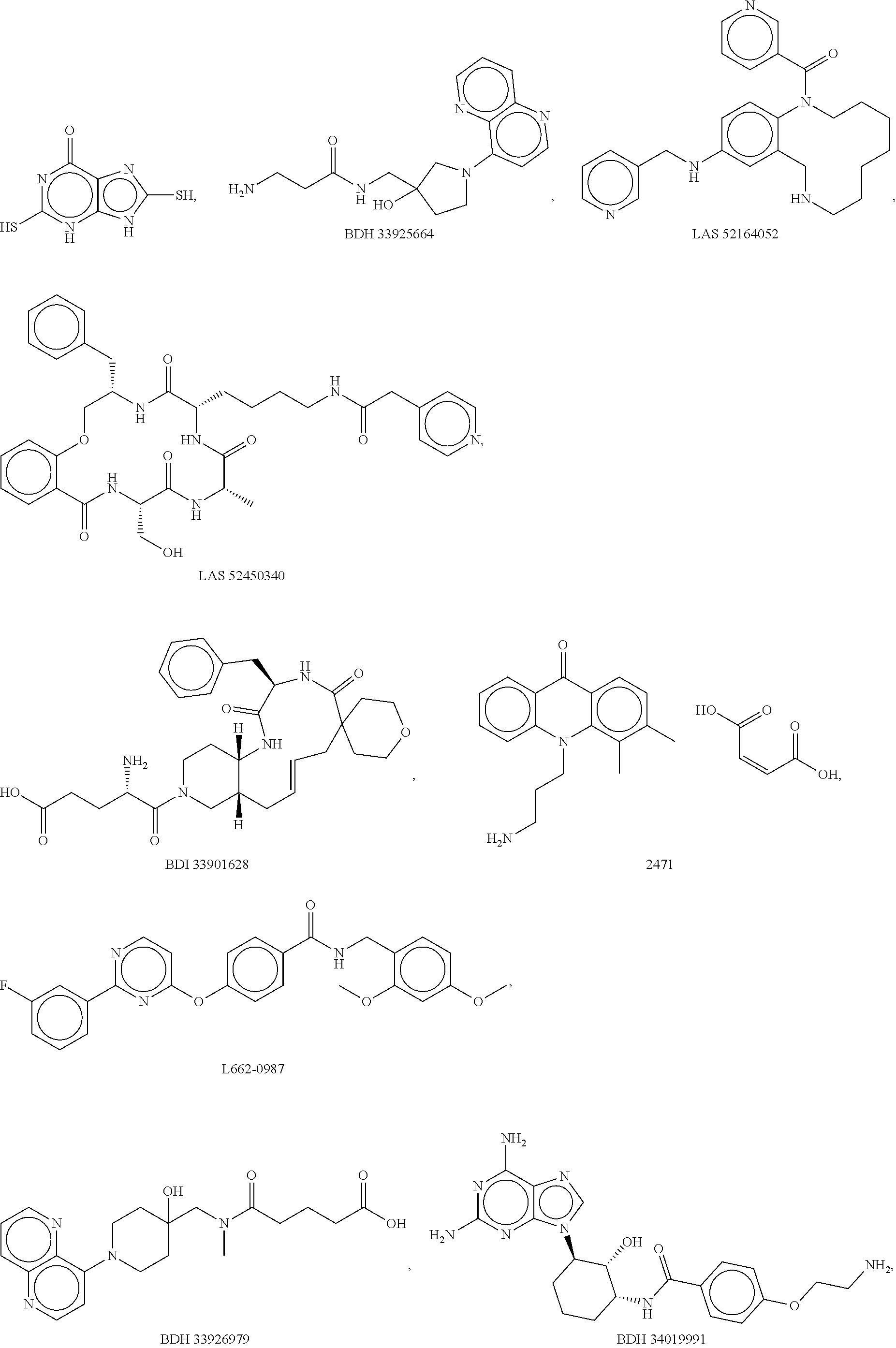

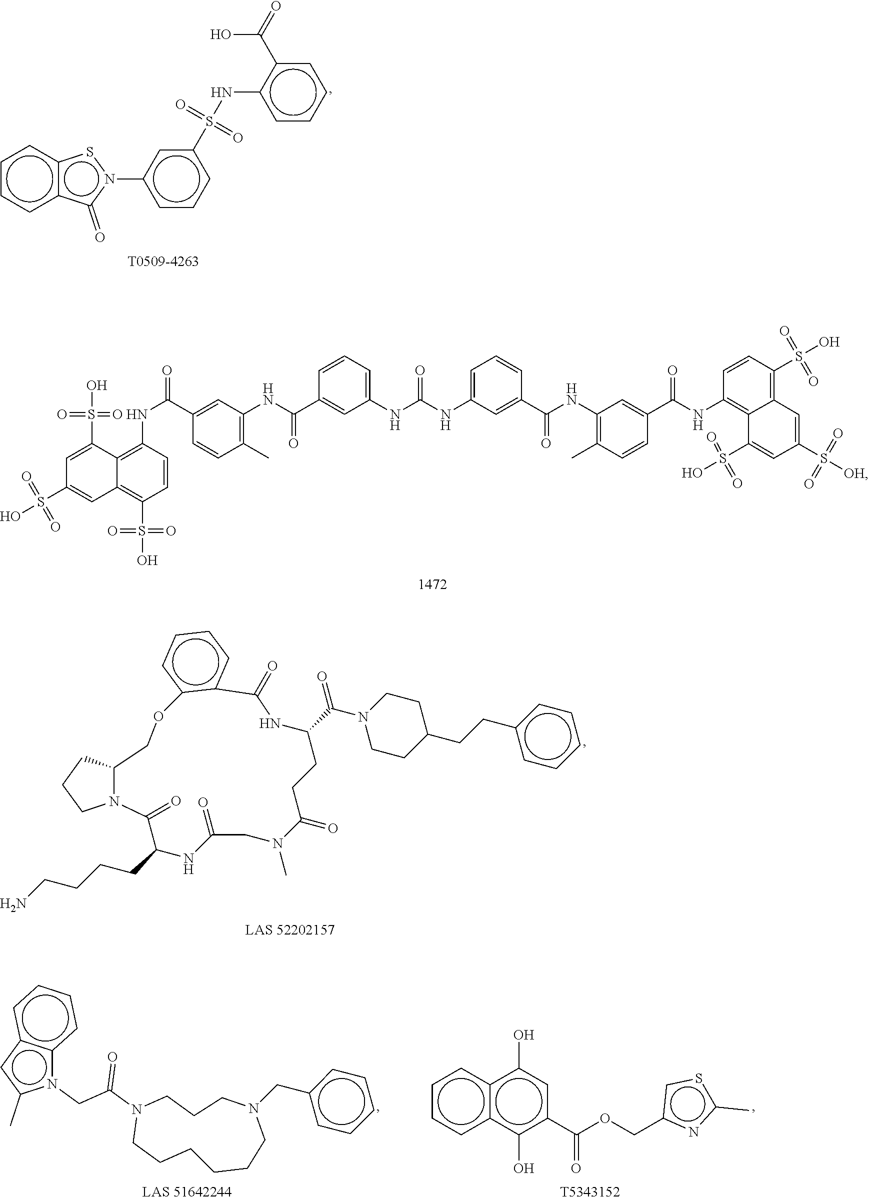

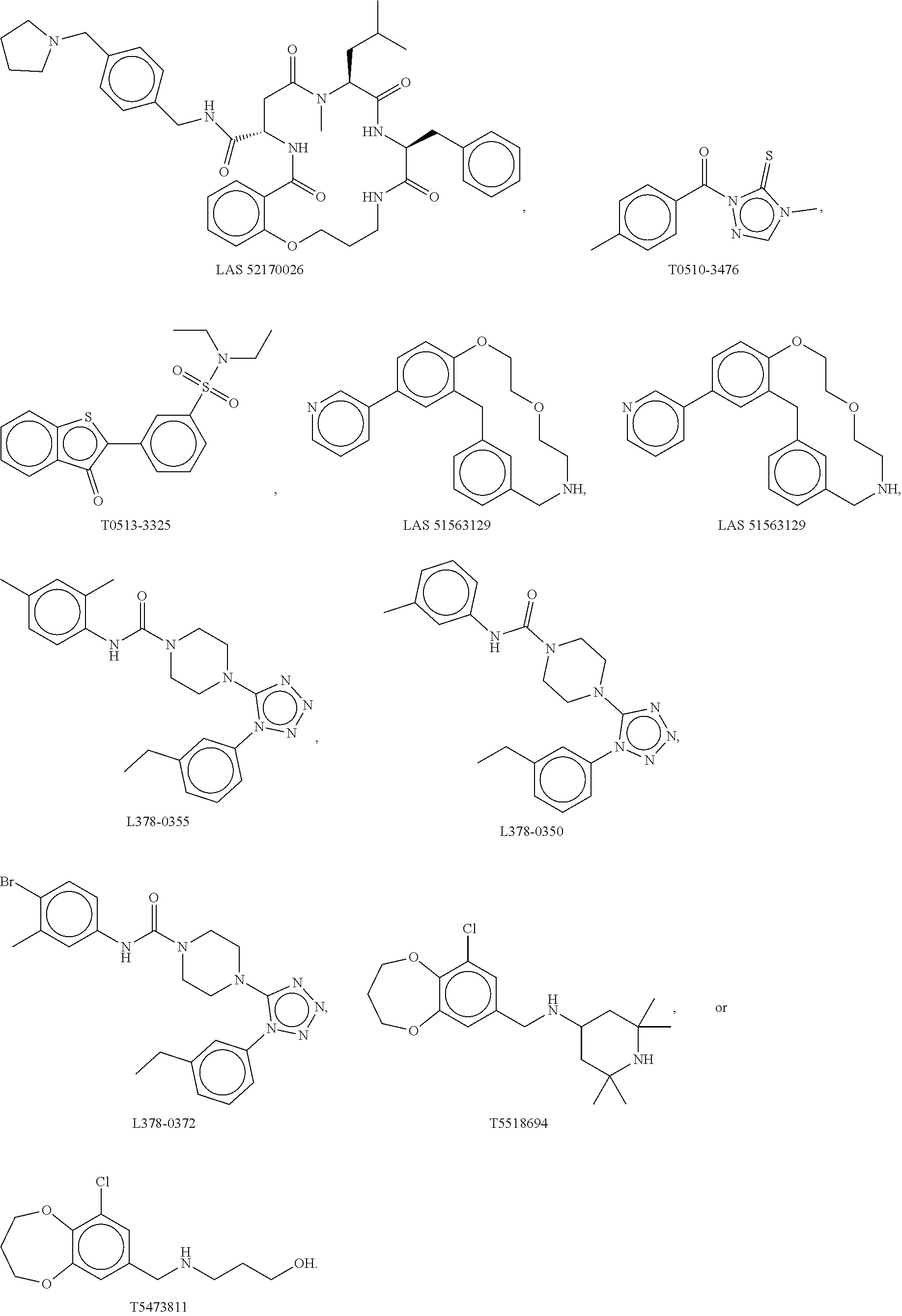

In certain embodiments, the inhibitor is a Cpf1 inhibitor. In embodiments, the inhibitor is according to the formula:

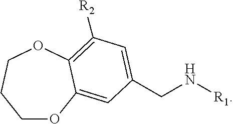

In embodiments, R₁ is a cycloalkyl, optionally substituted with one or more heteroatoms in the ring, and R₂ is halogen. As discussed herein, several approaches based on SAR studies and other chemical approaches can be used to vary substituents on the structure. In embodiments selected R1 is selected from

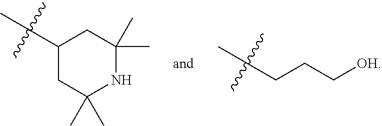 [SMILES: *C1CC(C)(C)NC(C)(C)C1 ; *CCCO]

In embodiments, R₂ is a halogen, in certain embodiments R₂ is Cl.

In embodiments, the inhibitor is according to the formula.

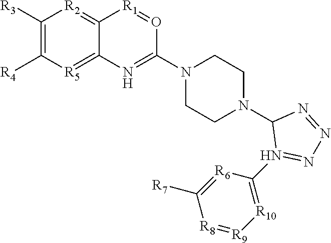

wherein R₁-R₁₀ is independently substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen.

In particular embodiments, the inhibitor is according to the formula:

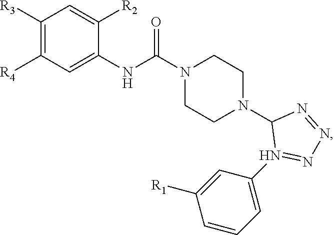

wherein R₁-R₄ is independently substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen.

In certain embodiments, R1 is alkyl, in preferred embodiments, ethyl. In certain embodiments, R₂ is H or CH₃, R₃ is selected from CH₃, methyl, and halogen, optionally bromine, and R₄ is methyl, or H.

In particular embodiments, the Cpf1 inhibitor is selected from:

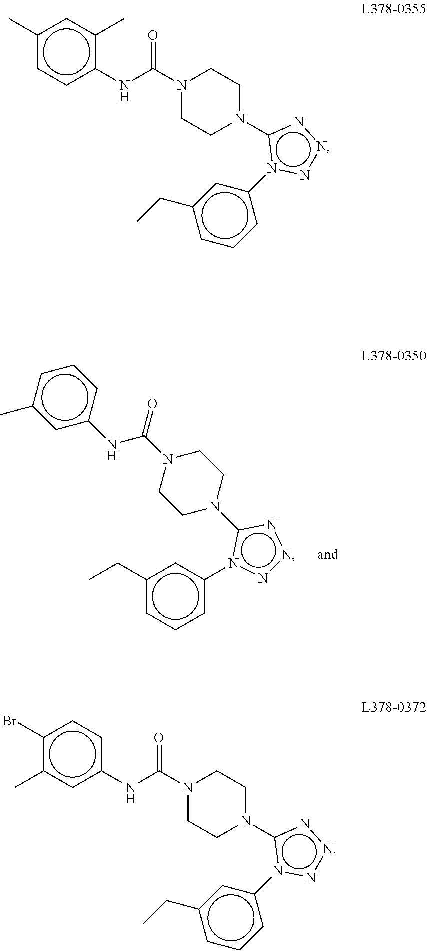

In certain embodiments, the Cpf1 inhibitor is selected from

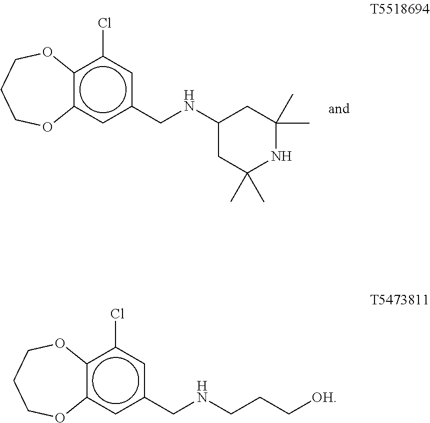

The compounds herein may be prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in RC. Larock, Comprehensive Organic Transformations, 2d. ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry (MS), or by chromatography such as high-performance liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes preparation of the Mosher's ester or amide derivative of the corresponding alcohol or amine, respectively. The absolute configuration of the ester or amide is then determined by proton and/or ¹⁹F NMR spectroscopy. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent compositions can be determined by one skilled in the art.

In general, small molecule compounds are known in the art or are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. For example, a library of 8,000 novel small molecules is available, which was created using combinatorial methods of Diversity-Oriented Synthesis (DOS) (Comer et al, Proc Natl Acad Sci USA 108, 6751 (Apr. 26, 2011; Lowe et al, J Org Chem 77, 7187 (Sep. 7, 2012); Marcaurelle et al, J Am Chem Soc 132, 16962 (Dec. 1, 2010)) to investigate chemical compounds not represented in traditional pharmaceutical libraries (Schreiber, S. L. (2000). Science 287, 1964-1969; Schreiber et al, Nat Biotechnol 28, 904 (September, 2010), each of which is herein incorporated by reference in their entirety). Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

SAR can be performed once compounds of interest are identified. One can use, for example, protocols for the C—H arylation of certain structures to generate valuable, stereochemically defined building blocks. Maetani et al. J Am Chem Soc. 2017 Aug. 16; 139(32):11300-11306, DOI:10.1021/jacs.7b06994. Analysis of stereochemistry-based structure-activity relationships (SAR) can provide whether substituents at one or more stereocenters are necessary for activity. Methods including C(sp³)-H functionalization methods can be utilized, including for scaffolds such as cyclopropanes (Zhang S.-Y.; Li Q.; He G.; Nack W. A.; Chen G. J. Am. Chem. Soc. 2013, 135, 12135-1214110.1021/ja406484v; Chan K. S.; Fu H.-Y.; Yu J.-Q. J. Am. Chem. Soc. 2015, 137, 2042-204610.1021/ja512529e as well as cyclobutanes, cyclopentanes, pyrrolidines, and piperidines. Such directed C(sp³) arylation is one approach for generation of compounds for further use in SAR studies. SAR studies can be studied, for example, using the eGFP assay described elsewhere herein. Synthesis and diversification of functionalized ring systems can be performed to evaluate use of scaffolds for generation of lead-like molecules. See, e.g., Lowe et al., J. Org. Chem. 2012, 77(17), pp. 7187-7211, DOI: 10.1021/jo300974j. Capping groups, aryl substituents, degrees of saturation, and electron withdrawing groups, for example, can be varied once lead compounds are identified. Dandapani, et al., doi: 10.1021/m1400403u, ACS Med. Chem. Lett. 2014, 5, 149-153. In this way, importance of stereochemistry and further refimements to structures can be made.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. USA. 90:6909, 1993; Erb et al., Proc. Natl. Acad. Sci. USA 91:11422, 1994; Zuckermann et al., J Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:63786382, 1990; Felici, J Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is identified as containing a compound of interest, further fractionation of the positive lead extract is necessary to isolate chemical constituents responsible for the observed effect. Thus, the goal of the extraction, fractionation, and purification process is the careful characterization and identification of a chemical entity within the crude extract that achieves a desired biological effect. Methods of fractionation and purification of such heterogenous extracts are known in the art.

Small molecules of the invention preferably have a molecular weight below 2,000 daltons, more preferably between 300 and 1,000 daltons, and most preferably between 400 and 700 daltons. It is preferred that these small molecules are organic molecules. Advantageously, small-molecule inhibitors can be cell-permeable, reversible, proteolytic stable, and non-immunogenic. Unlike genetic methods used to express protein-based anti-CRISPRs, small-molecule inhibitors exhibit fast kinetics, inhibiting enzymic activity in as little as a few minutes (Weiss et al., 2007), and allow precise temporal control. Small molecules can be synthesized on a large scale at low cost, with little batch-to-batch variability. Pharmacologic inhibition of intracellular proteins is usually accomplished using small molecules.

### Cas Proteins

In general, a CRISPR-Cas or CRISPR system as used herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e., the guide sequence is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein may advantageously be a codon optimized CRISPR effector protein. An example of a codon optimized sequence is in this instance a sequence optimized for expression in eukaryote, e.g., humans (i.e., being optimized for expression in humans), or for another eukaryote, animal or mammal as herein discussed; see, e.g., SaCas9 human codon optimized sequence in International Patent Publication No. WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the (E≤-actin promoter, the phosphoglycerol kinase (PGK) promoter, and the EF1(E±promoter. An advantageous promoter is the promoter is U6.

The RNA-guided nucleases herein may be identified by their proximity to cas1 genes, for example, though not limited to, within the region 20 kb from the start of the cas1 gene and 20 kb from the end of the cas1 gene. In certain embodiments, the RNA-guided nuclease comprises at least one HEPN domain and at least 500 amino acids, and protein is naturally present in a prokaryotic genome within 20 kb upstream or downstream of a Cas gene or a CRISPR array. Non-limiting examples of RNA-guided nucleases include Cas1, Cas1B, Cas2, Cas3, Cas4, Cas5, Cas6, Cas7, Cas8, Cas9 (also known as Csn1 and Csx12), Cas10, Cas12 (e.g., Cas12a, Cas12b, Cas12c, Cas12d), Cas13 (e.g., (Cas13a, Cas13b, Cas13c, Cas13d), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In one example embodiment, the RNA-guided nucleases may be the nuclease in any CRISPR-Cas system. In another example embodiment, the CRISPR system may be a class 2 CRISPR-Cas system, including Type II, Type V and Type VI systems. In certain example embodiments, the RNA-guided nuclease may be a is a Cas9, a Cas12a, Cas12b, Cas12c, Cas12d, Cas13a, Cas13b, Cas13c, or Cas13d system. For example, the RNA-guided nuclease may be Cas9, a Cas12a, Cas12b, Cas12c, Cas12d, Cas12k, a CasX, a CasY, a Cas(D, a MAD7, a Cas13a, Cas13b, Cas13c, or Cas13d.

In certain example embodiments, the RNA-guided nuclease is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related.

### Methods of Use

Small molecule inhibitors of RNA guided endonucleases (e.g., Cas9) were developed that have the potential to allow rapid, dosable, and/or temporal control of Cas9 activities. In some embodiments, provided herein include methods for inhibiting an RNA-guided endonuclease comprising contacting the RNA-guided endonuclease with one or more compounds described herein. In some examples, methods herein may include a method for treating a subject, comprising administering an RNA-guided endonuclease-RNA complex or a reagent causing expression of the RNA-guided endonuclease-RNA complex to the subject, and administering one or more compounds described herein.

The methods may be performed in vitro. Alternatively, or additionally, the methods may be performed in vivo. In some examples, the methods may be performed in a cell. The cell may be a germline cell. The cell may also be any type of cell, e.g., a stem cell such as an embryonic stem cell or a induced pluripotent stem cell. In certain examples, the methods may be performed in a cell in an organism (e.g., human, mammal, vertebrate, invertebrate, insect, plant). In some cases, the cell may be a prokaryotic cell, e.g., a bacterium. In certain cases, the cell may be a eukaryotic cell, e.g., a mammalian (e.g., human) cell, an insect cell, a plant cell, a fungal cell (e.g., a yeast cell).

Reports of small-molecule controlled Cas9 activity are present in literature (Senis et al., Biotechnol J 2014, 9, 1402-12; Wright et al., Proc Natl Acad Sci USA. 2015 Mar. 10; 112(10):2984-9; Gonzalez et al., Cell Stem Cell 2014, 15, 215-26; Davis et al., Nat Chem Biol 2015, 11, 316-8). However, none of them ensure dosability; the small molecules act merely as inducers of Cas9 activity. Further, most of these small molecule systems are not reversible upon removal of the small molecule (Zetsche et al., Nat Biotech 2015, 33, 139-142; Davis et al., Nat Chem Biol 2015, 11, 316-8), and therefore, do not allow precise temporal control in transcriptional regulatory technologies.

Small molecule inhibitors of RNA guided endonucleases (e.g., Cas9) have potential therapeutic uses for regulating genome editing technologies involving RNA guided endonucleases. Dosable control of the therapeutic activity of RNA guided endonucleases introduced into a subject or cell of a subject is important for effective genome editing therapeutic strategies. Small molecule inhibitors of RNA guided endonucleases can be administered to a subject undergoing RNA guided endonuclease-based gene therapy or any other RNA guided endonuclease-based therapy. In certain embodiments, the subject is a human or mammal. Small molecule inhibitors of RNA guided endonucleases eliminate or reduce undesirable off-target editing and chromosomal translocations when present at high concentrations Furthermore, small molecule inhibitors of RNA guided endonucleases can be used to rapidly terminate constitutively active Cas9, following on-target gene-editing.

Small molecule inhibitors of RNA guided endonucleases can also be used to regulate genome editing technologies in other organisms, including invertebrates, plants, and unicellular organisms (e.g., bacteria). Potential uses include regulating gene drives for entomological and agricultural uses. In addition, it is anticipated that Cas9 inhibitors will be valuable probes to understand the role of Cas9 in CRISPR-mediated bacterial immunity (e.g., spacer acquisition) (Nunez et al., Nature. 2015 Mar. 12; 519(7542):193-8; Heler et al., Nature 2015, 519, 199-202). Along similar lines, Cas9 inhibitors can be deployed for directed evolution of Cas9. It is hypothesized that Cas9 inhibitors will disrupt bacterial immunity against bacteriophages (or toxic DNA) by interfering with the CRISPR-Cas9-based immune surveillance system in bacteria. Akin to the development of antibiotic resistance, bacteria will be forced to evolve Cas9 protein. Accordingly, the inhibitors may also be used as an anti-infective agent.

Agents described herein, including analogs thereof, and/or agents discovered to have medicinal value using the methods described herein are useful as a drug for inhibiting RNA guided endonucleases (e.g., Cas9, Cpf1). For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms. Generally, amounts will be in the range of those used for other agents used in the treatment of disease.

The disclosed compounds may be administered alone (e.g., in saline or buffer) or using any delivery vehicles known in the art. For instance, the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers; Polymer rings; Proteosomes; Sodium Fluoride; Transgenic plants; Virosomes; Virus-like particles. Other delivery vehicles are known in the art and some additional examples are provided below.

The disclosed compounds may be administered by any route known, such as, for example, orally, transdermally, intravenously, cutaneously, subcutaneously, nasally, intramuscularly, intraperitoneally, intracranially, and intracerebroventricularly.

In certain embodiments, disclosed compounds are administered at dosage levels greater than about 0.001 mg/kg, such as greater than about 0.01 mg/kg or greater than about 0.1 mg/kg. For example, the dosage level may be from about 0.001 mg/kg to about 50 mg/kg such as from about 0.01 mg/kg to about 25 mg/kg, from about 0.1 mg/kg to about 10 mg/kg, or from about 1 mg/kg to about 5 mg/kg of subject body weight per day, one or more times a day, to obtain the desired therapeutic effect. It will also be appreciated that dosages smaller than about 0.001 mg/kg or greater than about 50 mg/kg (for example about 50-100 mg/kg) can also be administered to a subject.

In one embodiment, the compound is administered once-daily, twice-daily, or three-times daily. In one embodiment, the compound is administered continuously (e.g., every day) or intermittently (e.g., 3-5 days a week). In another embodiment, administration could be on an intermittent schedule.

Further, administration less frequently than daily, such as, for example, every other day may be chosen. In additional embodiments, administration with at least 2 days between doses may be chosen. By way of example only, dosing may be every third day, bi-weekly or weekly. As another example, a single, acute dose may be administered. Alternatively, compounds can be administered on a non-regular basis e.g., whenever symptoms begin. For any compound described herein the effective amount can be initially determined from animal models.

Toxicity and efficacy of the compounds can be determined by standard pharmaceutical procedures in cell cultures or experimental animals, e.g., for determining the LDso (the dose lethal to 50% of the population) and the EDso (the dose therapeutically effective in 50% of the population). The dose ratio between toxic and therapeutic effects is the therapeutic index, and it can be expressed as the ratio LDso/EDso. Compounds that exhibit large therapeutic indices may have a greater effect when practicing the methods as disclosed herein. While compounds that exhibit toxic side effects may be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage of the compounds disclosed herein for use in humans. The dosage of such agents lies within a range of circulating concentrations that include the ED50 with little or no toxicity. The dosage may vary within this range depending upon the dosage form employed and the route of administration utilized. For any compound used in the disclosed methods, the effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography. In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Multiple doses of the compounds are also contemplated.

The formulations disclosed herein are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of one or more disclosed compounds can be administered to a subject by any mode that delivers the compound(s) to the desired surface, e.g., mucosal, systemic. Administering the pharmaceutical composition of the present disclosure may be accomplished by any means known to the skilled artisan. Disclosed compounds may be administered orally, transdermally, intravenously, cutaneously, subcutaneously, nasally, intramuscularly, intraperitoneally, intracranially, or intracerebroventricularly.

### Method of Screening

Further disclosed herein include methods for screening, identifying, analyzing, and/or evaluating compounds that modulate (e.g., inhibit) RNA-guided nucleases. In some embodiments, such methods comprise a combination of biochemical and cellular assays.

The methods may be performed for screening, identifying, analyzing, and/or evaluating compounds that modulate (e.g., inhibit) any RNA-guided nucleases, such as RNA-guided nucleases in any CRISPR-Cas system. For example, the CRISPR system may be a class 2 CRISPR system, including Type II, Type V and Type VI systems. In certain example embodiments, the CRISPR system is a Cas9, a Cas12a, Cas12b, Cas12c, Cas12d, Cas13a, Cas13b, Cas13c, or Cas13d system. For example, the RNA-guided nuclease may be Cas9, a Cas12a, Cas12b, Cas12c, Cas12d, Cas13a, Cas13b, Cas13c, or Cas13d.

The methods may comprise one or more primary assays. The primary assays may be biochemical assays that assess the binding of the RNA-guided endonuclease with a target DNA. In some embodiments, the primary assay may be a fluorescence Polarization-based Assays.

The fluorescence Polarization-based Assay may monitor the change in the fluorescence polarization of the fluorophore-labelled PAM-rich target DNA (e.g., a 12PAM-DNA) upon binding to [Cas9:guideRNA] complex. In this assay, the complexation of [Cas9:guideRNA] to PAM-rich target DNA shows a dose-dependent increase in fluorophore polarization.

Fluorescence polarization is a useful technique to monitor the interaction between two molecules, including for example, Cas9-gRNA (ribonucleoprotein) complex and target DNA (e.g., 12PAM).

Fluorescence polarization may be used to measure the binding constants and kinetics of reactions that cause a change in the rotational time of the molecules. The technique is based on the change in the tumbling rate or mass after complexation. Smaller fragments may be fluorescently labeled and polarizations may be compared before and after complexation in the presence and absence of compounds. If the fluorophore is bound to a small molecule, the rate at which it tumbles can decrease significantly when it is bound tightly to a large protein. If the fluorophore is attached to the larger protein in a binding pair, the difference in polarization between bound and unbound states will be smaller (because the unbound protein will already be fairly stable and tumble slowly to begin with) and the measurement will be less accurate. The degree of binding is calculated by using the difference in anisotropy of the partially bound, free and fully bound (large excess of protein) states measured by titrating the two binding partners. If the fluorophore is bound to a relatively large molecule like a protein or an RNA, the change in the mobility accompanying folding can be used to study the dynamics of folding. This provides a measure of the dynamics of how the protein achieves its final, stable 3D shape.

The methods may further comprise one or more secondary assays. The secondary assays may be cell-based assays for testing the effect of candidate compounds on RNA-guided endonuclease activity in cells.

In some embodiments, the secondary assay may be an EGFP disruption assay. In such assay, a quantitative human cell-based reporter assay that enables rapid quantitation of targeted nuclease activities is used to characterize off-target cleavage of Cas9-based RNA guided endonucleases. In this assay, the activities of nucleases targeted to a single integrated EGFP reporter gene can be quantified by assessing loss of fluorescence signal in human U2OS.EGFP cells caused by inactivating frameshift insertion/deletion (indel) mutations introduced by error prone non-homologous end-joining (NHEJ) repair of nuclease-induced double-stranded breaks (DSBs).

In one protocol, U2OS.EGFP cells harboring a single integrated copy of an EGFP-PEST fusion gene are cultured (see e.g., Reyon et al., Nat Biotech 30, 460-465 (2012), which is herein incorporated by reference in its entirety). For transfections, 200,000 cells are Nucleofected with gRNA expression plasmid and pJDS246 together with 30 ng of a Td-tomato-encoding plasmid using the SE Cell Line 4D-Nucleofector™ X Kit (Lonza) according to the manufacturer's protocol. Cells are analyzed 2 days post-transfection using a BD LSRII flow cytometer. Transfections for optimizing gRNA/Cas9 plasmid concentration are performed in triplicate and all other transfections are performed in duplicate. PCR amplification is used for sequence verification of endogenous human genomic sites. PCR reactions are performed using Phusion Hot Start II high-fidelity DNA polymerase (NEB). Loci are amplified using touchdown PCR (98° C., 10 s; 72-62° C., −1° C./cycle, 15s; 72° C., 30 s] 10 cycles, [98° C., 10s; 62° C., 15s; 72° C., 30 s] 25 cycles). Alternatively, PCR for other targets is performed with 35 cycles at a constant annealing temperature of 68° C. or 72° C., and 3% DMSO or IM betaine, if necessary. PCR products are analyzed on a QIAXCEL capillary electrophoresis system to verify both size and purity. Validated products are treated with ExoSap-IT (Affymetrix) and sequenced by the Sanger method (MGH DNA Sequencing Core) to verify each target site.

In some embodiments, the secondary assay may be a fluorescence-based assay using cells expressing a single plasmid construct containing coding sequence for an RNA-guided endonuclease, a fluorescent peptide or protein, and a guide RNA. An example of such assays is disclosed in Moore R., Spinhirne et al., (2015). CRISPR-based self-cleaving mechanism for controllable gene delivery in human cells. Nucleic Acids Res 43, 1297-1303, which is incorporated by reference herein in its entirety.

In some embodiments, the secondary assay may be a loss-of-signal, non-homologous end joining (NHEJ) assay. An example of such assays is disclosed in Nguyen D P et al., (2016). Ligand-binding domains of nuclear receptors facilitate tight control of split CRISPR activity. Nat Commun 7, 12009, which is incorporated by reference herein in its entirety.

Other assays may be used in the methods discussed herein. In some embodiments, the methods may include a spinach transcription assay, which detects the activity of an RNA-guided endonuclease. In one embodiment, the level of transcription is suppressed by Cas9 nuclease activity in an in vitro assay. In various embodiments, the transcription assay involves expression of a nucleic acid aptamer that binds a molecular fluorophore to generate a fluorescent signal. Such aptamer-fluorophore combinations are known in the art, including for example, the Spinach aptamer having the sequence 5′-GGGAGACGCAACUGAAUGAAAUGGUGAAGGACGGGUCCAGGUGUGGCUGCUUCG GCAGUGCAGCUUGUUGAGUAGAGUGUGAGCUCCGCGUAACUAGUCGCGUCAC-3′ (SEQ ID NO: 1), and the fluorophore 4-(3,5-difluoro-4-hydroxybenzylidene)-1,2-dimethyl-1H-imidazol-5-one (DFHBI) (see, e.g., US20120252699 and US20140220560, each of which is incorporated herein in their entirety). In the Spinach assay, Cas9 can cleave the DNA template and thus inhibit in vitro transcription of the nucleic acid aptamer. In certain embodiments, the guide RNA targeting the Spinach aptamer has the sequence 5′-GCUAUAGGACGCGACCGAAAGUUUUAGAGCUAGAAAUAGCAAGUUAAAAUAAGG CUAGUCCGUUAUCAACUUGAAAAAGUGGCACCGAGUCGGUGCUUUU-3′ (SEQ ID NO: 2).

In the presence of fluorophore, suppression in transcription results in the reduction of RNA aptamer-fluorophore concentration and hence in the fluorescence signal. In vitro transcription reactions may comprise a purified linear DNA template containing a promoter operatively linked to a nucleic acid sequence encoding an RNA aptamer, ribonucleotide triphosphates, a buffer system (e.g., including DTT and magnesium ions, and an appropriate phage RNA polymerase (e.g., T7 polymerase).

In some embodiments, the methods may include a SURVEYOR nuclease assay. In various embodiments, a SURVEYOR nuclease assay is used to assess genome modification (see e.g., U.S. Patent Publication No. US 20150356239, which is herein incorporated by reference in its entirety. In one protocol, 293FT cells are transfected with plasmid DNA. Cells were incubated at 37° C. for 72 hours post-transfection prior to genomic DNA extraction. Genomic DNA is extracted using the QuickExtract DNA Extraction Solution (Epicentre) following the manufacturer's protocol. Briefly, pelleted cells are resuspended in QuickExtract solution and incubated at 65° C. for 15 minutes and 98° C. for 10 minutes. The genomic region flanking the CRISPR target site for each gene is PCR amplified, and products are purified using QiaQuick Spin Column (Qiagen) following the manufacturer's protocol. 400 ng total of the purified PCR products are mixed with 2 μL 10× Taq DNA Polymerase PCR buffer (Enzytrsaties) and ultrapure water to a final volume of 20 μL and subjected to a re-annealing process to enable heteroduplex formation: 95° C. for 10 min, 95° C. to 85° C. ramping at −2° C./s, 85° C. to 25° C. at −0.25° C./s, and 25° C. hold for 1 minute. After re-annealing, products are treated with SURVEYOR nuclease and SURVEYOR enhancer S (Transgenomics) following the manufacturer's recommended protocol and analyzed on 4-20% Novex TBE poly-acrylamide gels (Life Technologies). Gels are stained with SYBR Gold DNA stain (Life Technologies) for 30 minutes and imaged with a Gel Doe gel imaging system (Bio-rad). Quantification is based on relative band intensities.

### Kits

The present compositions, e.g., compounds and/or pharmaceutical formulations may be assembled into kits or pharmaceutical systems. The kits can include instructions for the treatment regime, reagents, equipment (test tubes, reaction vessels, needles, syringes, etc.) and standards for calibrating or conducting the treatment. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### Formulations

The compounds herein may be in the form of a pharmaceutically acceptable salt. When used in medicine the salts should be pharmaceutically acceptable, but non-pharmaceutically acceptable salts may conveniently be used to prepare pharmaceutically acceptable salts thereof. Examples of pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group. Suitable buffering agents include: acetic acid and a salt (about 1-2% w/v); citric acid and a salt (about 1-3% w/v); boric acid and a salt (about 0.5-2.5% w/v); and phosphoric acid and a salt (about 0.8-2% w/v). Suitable preservatives include benzalkonium chloride (about 0.003-0.03% w/v); chlorobutanol (about 0.3-0.9% w/v); parabens (about 0.01-0.25% w/v) and thimerosal (about 0.004-0.02% w/v).

Also disclosed herein may be pharmaceutical formulations that comprise an effective amount of one or more compounds disclosed herein optionally included in a pharmaceutically acceptable carrier. The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds, and with each other, in a manner such that there is no interaction which would substantially impair the desired pharmaceutical efficiency.

For oral administration, one or more compounds can be formulated readily by combining the active compound(s) with pharmaceutically acceptable carriers well known in the art. Such carriers enable the compounds to be formulated as tablets, pills, dragees, capsules, liquids, gels, syrups, slurries, suspensions and the like, for oral ingestion by a subject to be treated.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also contemplated are oral dosage forms of one or more disclosed compounds. The compound(s) may be chemically modified so that oral delivery of the derivative is efficacious. Generally, the chemical modification contemplated is the attachment of at least one moiety to the compound itself, where said moiety permits (a) inhibition of proteolysis; and (b) uptake into the blood stream from the stomach or intestine. Also desired is the increase in overall stability of the compound(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane. In some aspects, for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach yet will release the material in the duodenum or elsewhere in the intestine. In some aspects, the release will avoid the deleterious effects of the stomach environment, either by protection of the compound or by release of the biologically active material beyond the stomach environment, such as in the intestine.

To ensure full gastric resistance a coating impermeable to at least pH 5.0 is important. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The disclosed compounds can be included in the formulation as fine multiparticulates in the form of granules or pellets of particle size about 1 mm. The formulation of the material for capsule administration could also be as a powder, lightly compressed plugs or even as tablets. The compound could be prepared by compression.

Colorants and flavoring agents may all be included. For example, the compound may be formulated (such as by liposome or microsphere encapsulation) and then further contained within an edible product, such as a refrigerated beverage containing colorants and flavoring agents.

One may dilute or increase the volume of compound delivered with an inert material. These diluents could include carbohydrates, especially mannitol, a-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell. Disintegrants may be included in the formulation of the therapeutic into a solid dosage form Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants is the insoluble cationic exchange resins. Powdered gums may be used as disintegrants and as binders, and these can include powdered gums such as agar, Karaya or tragacanth. Alginic acid and its sodium salt are also useful as disintegrants.

Binders may be used to hold the therapeutic together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the compound to prevent sticking during the formulation process. Lubricants may be used as a layer between the compound and the die wall, and these can include, but are not limited to, stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the compound into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds of the disclosure. The compound is delivered to the lungs of a mammal while inhaling and traverses across the lung epithelial lining to the blood stream using methods well known in the art. Contemplated for use in the practice of methods disclosed herein are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of these methods are the Ultravent nebulizer, manufactured by Mallinckrodt, Inc., St. Louis, Missouri; the Acorn II nebulizer, manufactured by Marquest Medical Products, Englewood, Colorado; the Ventolin metered dose inhaler, manufactured by Glaxo Inc., Research Triangle Park, North Carolina; and the Spinhaler powder inhaler, manufactured by Fisons Corp., Bedford, Massachusetts.

All such devices require the use of formulations suitable for the dispensing of compound. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified compound may also be prepared in different formulations depending on the type of chemical modification or the type of device employed. Formulations suitable for use with a nebulizer, either jet or ultrasonic, will typically comprise compound dissolved in water at a concentration of about 0.1 to about 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the compound suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound and may also include a bulking agent, such as lactose, sorbitol, sucrose, or mannitol in amounts which facilitate dispersal of the powder from the device, e.g., about 50 to about 90% by weight of the formulation. The compound should most advantageously be prepared in particulate form with an average particle size of less than 10 mm (or microns), such as about 0.5 to about 5 mm, for an effective delivery to the distal lung.

Nasal delivery of a disclosed compound is also contemplated. Nasal delivery allows the passage of a compound to the blood stream directly after administering the therapeutic product to the nose, without the necessity for deposition of the product in the lung.

Formulations for nasal delivery include those with dextran or cyclodextran. For nasal administration, a useful device is a small, hard bottle to which a metered dose sprayer is attached. In one embodiment, the metered dose is delivered by drawing the pharmaceutical composition solution into a chamber of defined volume, which chamber has an aperture dimensioned to aerosolize and aerosol formulation by forming a spray when a liquid in the chamber is compressed. The chamber is compressed to administer the pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. In some aspects, the nasal inhaler will provide a metered amount of the aerosol formulation, for administration of a measured dose of the drug.

The compound, when it is desirable to deliver them systemically, may be formulated for parenteral administration by injection, e.g., by bolus injection or continuous infusion.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions.

Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

The invention is further described in the following examples, which do not limit the scope of the invention described in the claims.

### EXAMPLES

### Example 1

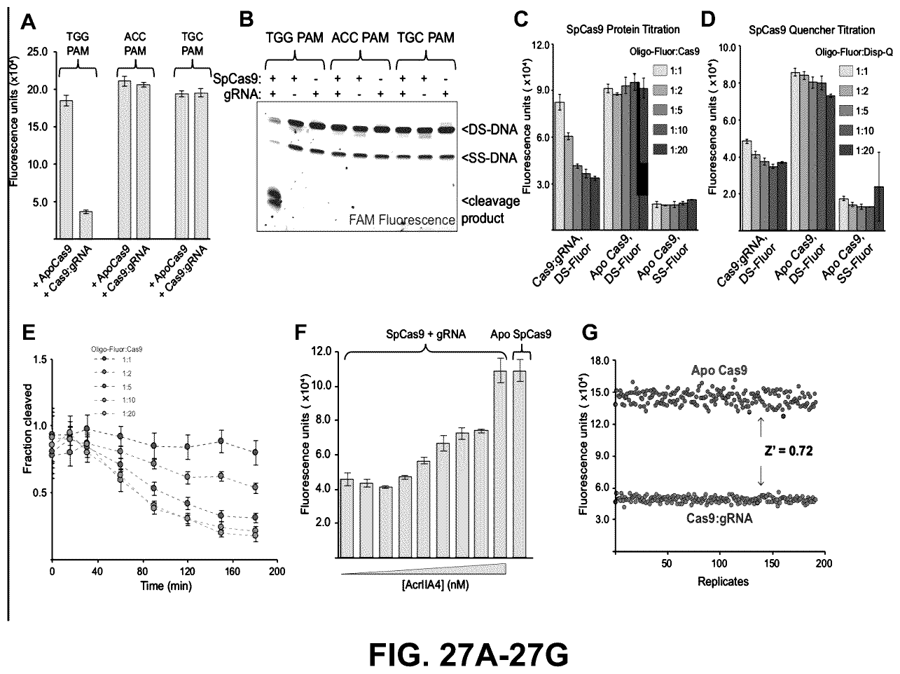

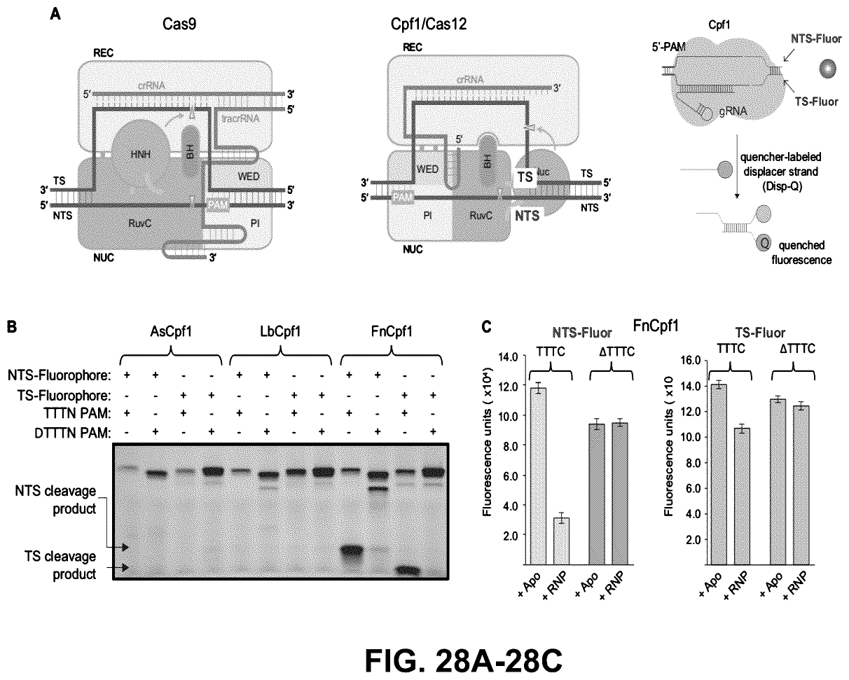

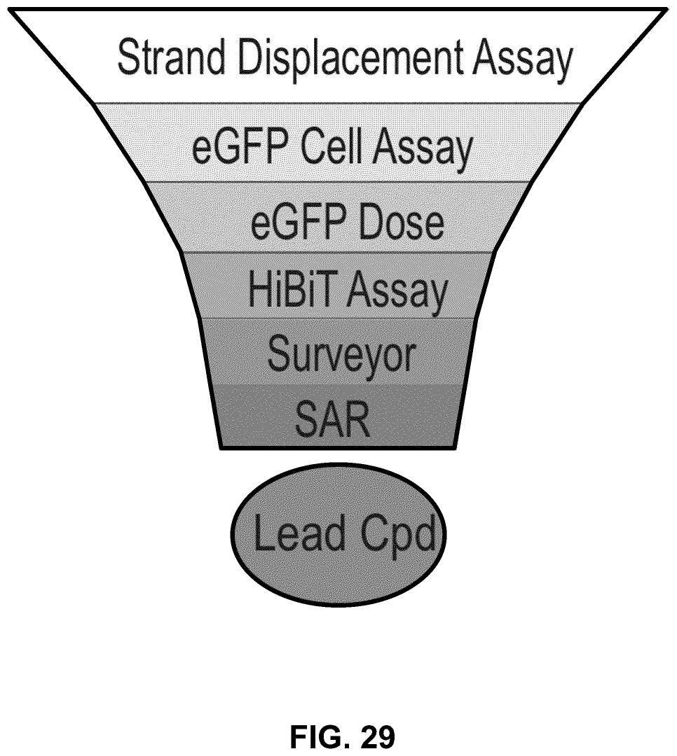

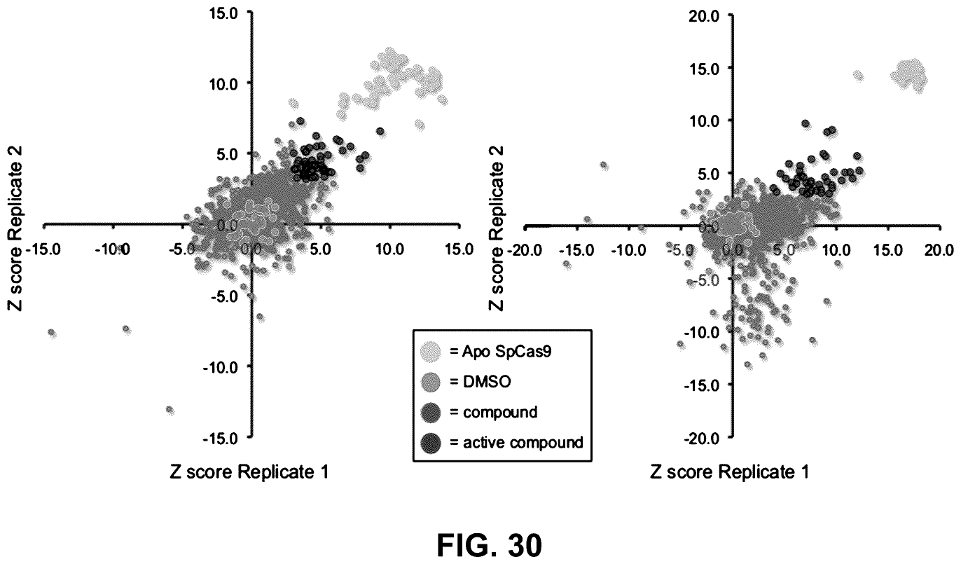

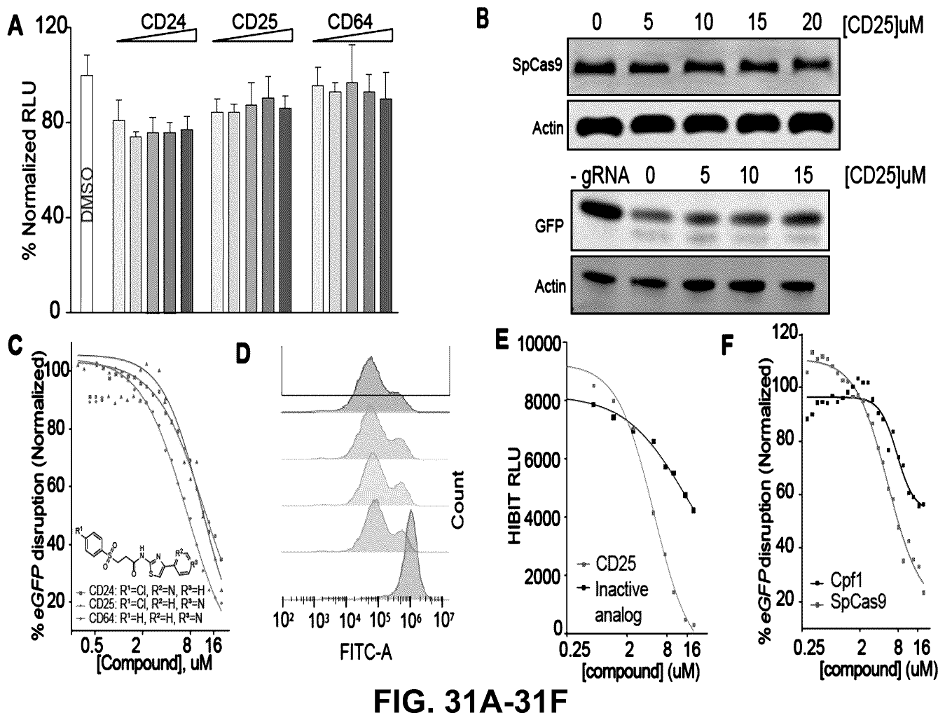

Screening of inhibitors was via a high throughput pipeline, as depicted in FIG. 29. The first screening is via a strand displacement assay (SDA) for detecting nuclease activity (FIG. 27A, 28A). The Cpf1/Cas12 protein/DNA complex has a different structure from Cas9 and requires optimization of the potential fluorophore attachment sites (Yamano, T et al., Cell, 2016). (FIG. 28A). Next screened compounds are evaluated in an eGFP cell assay, and for eGFP dose. Those compounds are further narrowed via a HiBiT assay and finally evaluated in the surveyor assay. SAR evaluations are performed to arrive at lead compounds for further evaluations and variations. General methodology for the assays is as described in at [0253]-[0301] of U.S. Patent Application 62/831,143 filed Apr. 8, 2019, incorporated herein by reference.

### SpCas9 Inhibitors

SpCas9 is a 160 kDa protein, with 80 nt tracRNA, and 20 nt crRNA that recognizes 3′ NGG PAM and has many known anti-CRISPR proteins. Initial screening of 42419 compounds resulted in a Cherrypick of SpCas9 inhibitors as provided in Table 2A.

TABLE 2A
Sp Cas9 Cherrypick of 42419 compounds

| | | | | | | | | | Secondary | |
|---|---|---|---|---|---|---|---|---|---|---|
| | | | | Primary Screen (150 min) | | | | | Screen: | |
| SpCas9 compounds | | | | | | | | Average | Autofluorescence | |
| Cherrypick 42419 | | | | avg z | stdev z | Normalized | Normalized | Normalized | avg z | stdev z |
| | Vendor_ | Posi- | Cherry | score | score | Z | Z | Z | score | score |
| Library | ID | tive | Pick | primary | primary | score 1 | score 2 | score | secondary | secondary |
| ChemDiv6 | 3683- | M | C | 4.50 | 1.51716235 | 0.25 | 0.20 | 0.22 | 1.09 | 0.00 |
| | 0412 | | | | | | | | | |
| ChemDiv6 | 3759- | M | C | 6.50 | 2.36423737 | 0.35 | 0.29 | 0.32 | 1.03 | 1.68 |
| | 1447 | | | | | | | | | |
| ChemDiv6 | 3772- | M | C | 4.22 | 1.69805817 | 0.22 | 0.19 | 0.20 | −0.50 | 0.70 |
| | 4201 | | | | | | | | | |
| ChemDiv6 | 4116- | M | C | 3.70 | 0.98479413 | 0.14 | 0.18 | 0.16 | 0.89 | 0.32 |
| | 0061 | | | | | | | | | |
| ChemDiv6 | 4149- | M | C | 4.11 | 0.74423601 | 0.22 | 0.15 | 0.18 | −0.25 | 0.58 |
| | 0237 | | | | | | | | | |
| ChemDiv6 | 4168- | M | C | 3.79 | 0.72763309 | 0.20 | 0.13 | 0.17 | 0.37 | 1.25 |
| | 2546 | | | | | | | | | |
| ChemDiv6 | 4358- | S | C | 5.18 | 0.19361382 | 0.25 | 0.20 | 0.23 | −0.30 | 0.26 |
| | 3320 | | | | | | | | | |
| ChemDiv6 | 4369- | W | C | 3.42 | 0.30809693 | 0.17 | 0.13 | 0.15 | −1.33 | 0.09 |
| | 0026 | | | | | | | | | |
| ChemDiv6 | 4296- | W | C | 3.68 | 0.20549066 | 0.17 | 0.16 | 0.16 | 1.05 | 0.91 |
| | 0578 | | | | | | | | | |
| ChemDiv6 | 4321- | M | C | 4.36 | 0.61751539 | 0.18 | 0.19 | 0.19 | −0.05 | 0.15 |
| | 0064 | | | | | | | | | |
| ChemDiv6 | 4130- | W | C | 3.47 | 0.28009947 | 0.17 | 0.13 | 0.15 | 1.18 | 0.14 |
| | 5308 | | | | | | | | | |
| ChemDiv6 | 4321- | W | C | 3.17 | 0.05509781 | 0.15 | 0.13 | 0.14 | 0.10 | 0.57 |
| | 0229 | | | | | | | | | |
| ChemDiv6 | 4149- | M | C | 4.25 | 0.15586994 | 0.20 | 0.17 | 0.19 | 0.90 | 0.22 |
| | 0360 | | | | | | | | | |
| ChemDiv6 | 4378- | W | C | 3.72 | 0.01261325 | 0.17 | 0.15 | 0.16 | 0.85 | 1.18 |
| | 0392 | | | | | | | | | |
| ChemDiv6 | 4300- | W | C | 3.55 | 0.18300505 | 0.17 | 0.14 | 0.16 | 0.11 | 0.27 |
| | 0717 | | | | | | | | | |
| ChemDiv6 | 4321- | M | C | 4.20 | 0.52778555 | 0.18 | 0.19 | 0.18 | 0.55 | 1.50 |
| | 1201 | | | | | | | | | |
| ChemDiv6 | 4326- | M | C | 3.84 | 0.33556424 | 0.19 | 0.15 | 0.17 | 2.05 | 0.96 |
| | 0044 | | | | | | | | | |
| ChemDiv6 | 6850- | W | C | 3.17 | 0.00851395 | 0.06 | 0.06 | 0.06 | −1.70 | 1.02 |
| | 0211 | | | | | | | | | |
| ChemDiv6 | 8010- | M | C | 6.77 | 5.30286842 | 0.14 | 0.06 | 0.10 | 0.89 | 0.19 |
| | 1547 | | | | | | | | | |
| ChemDiv6 | C226 | M | C | 4.44 | 1.65791698 | 0.19 | 0.15 | 0.17 | 1.19 | 0.31 |
| | 0554 | | | | | | | | | |
| ChemDiv6 | C226- | M | C | 4.04 | 0.95463201 | 0.16 | 0.16 | 0.16 | −0.06 | 0.39 |
| | 3419 | | | | | | | | | |
| ChemDiv6 | C226- | M | | 5.28 | 1.72098393 | 0.22 | 0.19 | 0.20 | −0.67 | 0.65 |
| | 0892* | | | | | | | | | |
| ChemDiv6 | C226- | M | | 4.60 | 2.19369699 | 0.20 | 0.14 | 0.17 | −0.61 | 1.22 |
| | 0322* | | | | | | | | | |
| ChemDiv6 | C226- | M | | 4.24 | 1.21799374 | 0.17 | 0.16 | 0.16 | 0.92 | 1.02 |
| | 0550* | | | | | | | | | |
| ChemDiv6 | C226- | M | C | 6.10 | 2.81137539 | 0.27 | 0.19 | 0.23 | 0.71 | 0.33 |
| | 1003 | | | | | | | | | |
| ChemDiv6 | C226- | M | C | 4.75 | 2.29479821 | 0.21 | 0.15 | 0.18 | 0.94 | 0.09 |
| | 2834 | | | | | | | | | |
| ChemDiv6 | C226- | M | C | 5.33 | 1.3849278 | 0.21 | 0.20 | 0.21 | 0.51 | 1.01 |
| | 1119 | | | | | | | | | |
| ChemDiv6 | C499- | M | C | 3.98 | 1.10107481 | 0.16 | 0.12 | 0.14 | −0.49 | 0.55 |
| | 0394 | | | | | | | | | |
| ChemDiv6 | D132- | W | C | 3.32 | 0.36638589 | 0.21 | 0.19 | 0.20 | 0.39 | 1.10 |
| | 0053 | | | | | | | | | |
| ChemDiv6 | D132- | M | C | 4.97 | 0.48566566 | 0.31 | 0.28 | 0.29 | 1.11 | 1.47 |
| | 0061 | | | | | | | | | |
| ChemDiv6 | D216- | W | C | 3.09 | 0.07257226 | 0.14 | 0.14 | 0.14 | −1.47 | 1.98 |
| | 0223 | | | | | | | | | |
| ChemDiv6 | D343- | M | | 4.42 | 0.41182841 | 0.31 | 0.27 | 0.29 | 1.02 | 0.95 |
| | 0010* | | | | | | | | | |
| ChemDiv6 | D343- | M | | 4.22 | 0.83067671 | 0.27 | 0.27 | 0.27 | −0.72 | 0.55 |
| | 0078* | | | | | | | | | |
| ChemDiv6 | D343- | M | | 4.44 | 1.14847135 | 0.27 | 0.30 | 0.28 | 0.42 | 0.79 |
| | 0052* | | | | | | | | | |
| ChemDiv6 | D343- | S | | 6.34 | 0.68525018 | 0.43 | 0.39 | 0.41 | −0.55 | 0.18 |
| | 0005* | | | | | | | | | |
| ChemDiv6 | E143- | M | C | 4.14 | 0.21715281 | 0.32 | 0.23 | 0.27 | −0.08 | 0.69 |
| | 0211 | | | | | | | | | |
| ChemDiv6 | E947- | M | C | 3.67 | 0.57102064 | 0.19 | 0.21 | 0.20 | −1.41 | 1.58 |
| | 0325 | | | | | | | | | |
| ChemDiv6 | E947- | W | C | 3.61 | 0.15757876 | 0.18 | 0.23 | 0.20 | −0.48 | 1.31 |
| | 0476 | | | | | | | | | |
| ChemDiv6 | E948- | W | C | 3.41 | 0.28203932 | 0.17 | 0.21 | 0.19 | −0.56 | 0.57 |
| | 0657 | | | | | | | | | |
| ChemDiv6 | E955- | W | C | 3.26 | 0.35393818 | 0.29 | 0.25 | 0.27 | 0.17 | 0.12 |
| | 0758 | | | | | | | | | |
| ChemDiv6 | E955- | W | C | 3.32 | 0.20894574 | 0.27 | 0.29 | 0.28 | 0.05 | 0.43 |
| | 0793 | | | | | | | | | |
| ChemDiv6 | E975- | M | C | 4.80 | 0.9197722 | 0.39 | 0.22 | 0.30 | −0.47 | 0.43 |
| | 1424 | | | | | | | | | |
| ChemDiv6 | F046- | M | C | 4.11 | 0.25584597 | 0.27 | 0.20 | 0.24 | −0.16 | 0.07 |
| | 0136 | | | | | | | | | |
| ChemDiv6 | E999- | M | C | 4.24 | 0.0843577 | 0.29 | 0.20 | 0.25 | −0.95 | 0.15 |
| | 1192 | | | | | | | | | |
| ChemDiv6 | F052- | M | C | 4.03 | 0.23739567 | 0.29 | 0.18 | 0.24 | 0.44 | 0.88 |
| | 0073 | | | | | | | | | |
| ChemDiv6 | F067- | M | C | 3.78 | 1.02442583 | 0.21 | 0.21 | 0.21 | 0.18 | 0.04 |
| | 0393 | | | | | | | | | |
| ChemDiv6 | F069- | W | C | 3.43 | 0.09745055 | 0.23 | 0.17 | 0.20 | −0.11 | 0.18 |
| | 0372 | | | | | | | | | |
| ChemDiv6 | F058- | W | C | 3.32 | 0.44674083 | 0.21 | 0.17 | 0.19 | 0.25 | 1.23 |
| | 1251 | | | | | | | | | |
| ChemDiv6 | F046- | M | C | 4.35 | 1.60820814 | 0.22 | 0.26 | 0.24 | −0.11 | 0.50 |
| | 0205 | | | | | | | | | |
| ChemDiv6 | G330- | M | C | 3.74 | 0.50751755 | 0.16 | 0.15 | 0.16 | −0.46 | 0.52 |
| | 0065 | | | | | | | | | |
| ChemDiv6 | G574- | M | C | 4.42 | 1.78073479 | 0.18 | 0.15 | 0.16 | −0.15 | 0.53 |
| | 0192 | | | | | | | | | |
| ChemDiv6 | G585- | M | C | 4.06 | 1.30543999 | 0.15 | 0.15 | 0.15 | −2.00 | 1.77 |
| | 0445 | | | | | | | | | |
| ChemDiv6 | G747- | M | C | 4.79 | 1.9676614 | 0.21 | 0.23 | 0.22 | 0.88 | 0.12 |
| | 0581 | | | | | | | | | |
| ChemDiv6 | G755- | W | C | 3.59 | 0.25938521 | 0.12 | 0.19 | 0.15 | 0.09 | 1.12 |
| | 0280 | | | | | | | | | |
| ChemDiv6 | G755- | M | C | 4.36 | 0.78124861 | 0.18 | 0.19 | 0.18 | 0.84 | 0.16 |
| | 0493 | | | | | | | | | |
| ChemDiv6 | G756- | M | C | 3.93 | 1.23425806 | 0.17 | 0.15 | 0.16 | 0.46 | 0.43 |
| | 0013 | | | | | | | | | |
| ChemDiv6 | G756- | W | C | 3.42 | 0.54171716 | 0.14 | 0.15 | 0.14 | −0.55 | 1.49 |
| | 0029 | | | | | | | | | |
| ChemDiv6 | G756- | W | C | 3.16 | 0.12551758 | 0.11 | 0.16 | 0.14 | 0.10 | 0.78 |
| | 0163 | | | | | | | | | |
| ChemDiv6 | G756- | M | C | 3.70 | 0.85612637 | 0.15 | 0.15 | 0.15 | −0.05 | 0.68 |
| | 0570 | | | | | | | | | |
| ChemDiv6 | G756- | M | C | 4.02 | 1.40427659 | 0.18 | 0.15 | 0.16 | 0.19 | 0.96 |
| | 0575 | | | | | | | | | |
| ChemDiv6 | G781- | M | C | 5.16 | 0.66583216 | 0.24 | 0.21 | 0.22 | 0.70 | 1.41 |
| | 1644 | | | | | | | | | |
| ChemDiv6 | G786- | M | C | 4.36 | 0.54382722 | 0.20 | 0.17 | 0.19 | 0.72 | 2.51 |
| | 0265 | | | | | | | | | |
| ChemDiv6 | G793- | M | C | 4.91 | 0.53474727 | 0.14 | 0.17 | 0.16 | −1.15 | 0.41 |
| | 0303 | | | | | | | | | |
| ChemDiv6 | G794- | M | C | 4.41 | 0.95578682 | 0.13 | 0.14 | 0.14 | 0.46 | 0.49 |
| | 2464 | | | | | | | | | |
| ChemDiv6 | G795- | M | | 4.45 | 1.4859604 | 0.15 | 0.13 | 0.14 | −0.39 | 1.08 |
| | 0268* | | | | | | | | | |
| ChemDiv6 | G795- | M | C | 4.54 | 1.7670296 | 0.15 | 0.13 | 0.14 | −0.65 | 0.11 |
| | 0607 | | | | | | | | | |
| ChemDiv6 | G795- | M | C | 5.01 | 2.50912098 | 0.18 | 0.12 | 0.15 | 1.59 | 0.37 |
| | 0711 | | | | | | | | | |
| ChemDiv6 | G796- | M | | 4.19 | 1.53276498 | 0.14 | 0.12 | 0.13 | 0.43 | 0.78 |
| | 0616* | | | | | | | | | |
| ChemDiv6 | G796- | M | C | 4.24 | 1.39282656 | 0.14 | 0.12 | 0.13 | 0.64 | 2.52 |
| | 0802 | | | | | | | | | |
| ChemDiv6 | G794- | M | C | 3.85 | 1.19079951 | 0.12 | 0.11 | 0.12 | −0.21 | 0.90 |
| | 0521 | | | | | | | | | |
| ChemDiv6 | G795- | M | C | 3.65 | 0.57080088 | 0.11 | 0.12 | 0.12 | 0.60 | 0.98 |
| | 0613 | | | | | | | | | |
| ChemDiv6 | G795- | W | C | 3.76 | 0.20780656 | 0.10 | 0.15 | 0.12 | 0.31 | 0.73 |
| | 0628 | | | | | | | | | |
| ChemDiv6 | G795- | M | C | 4.23 | 0.64235442 | 0.12 | 0.14 | 0.13 | −0.47 | 0.46 |
| | 0632 | | | | | | | | | |
| ChemDiv6 | G827- | M | C | 3.65 | 0.79600875 | 0.16 | 0.13 | 0.14 | −0.16 | 0.30 |
| | 0085 | | | | | | | | | |
| ChemDiv6 | G830- | W | C | 3.46 | 0.16667114 | 0.14 | 0.14 | 0.14 | 0.13 | 0.22 |
| | 0919 | | | | | | | | | |
| ChemDiv6 | G831- | M | C | 3.98 | 0.22427899 | 0.16 | 0.16 | 0.16 | −0.59 | 0.00 |
| | 0080 | | | | | | | | | |
| ChemDiv6 | G831- | M | C | 4.04 | 0.54789973 | 0.17 | 0.15 | 0.16 | −0.81 | 1.39 |
| | 0220 | | | | | | | | | |
| ChemDiv6 | G832- | M | C | 3.58 | 0.69494923 | 0.12 | 0.17 | 0.14 | 0.75 | 0.36 |
| | 0021 | | | | | | | | | |
| ChemDiv6 | G832- | M | C | 3.79 | 0.31483935 | 0.15 | 0.15 | 0.15 | −0.13 | 0.05 |
| | 0421 | | | | | | | | | |
| ChemDiv6 | G830- | M | C | 3.61 | 0.62865196 | 0.15 | 0.13 | 0.14 | −0.06 | 0.85 |
| | 0920 | | | | | | | | | |
| ChemDiv6 | G832- | M | C | 4.40 | 0.60901166 | 0.15 | 0.20 | 0.18 | 0.88 | 2.14 |
| | 0036 | | | | | | | | | |
| ChemDiv6 | G832- | W | C | 3.17 | 0.03631013 | 0.12 | 0.13 | 0.13 | −0.03 | 1.00 |
| | 0103 | | | | | | | | | |
| ChemDiv6 | G856- | W | | 3.48 | 0.64055469 | 0.16 | 0.10 | 0.13 | −0.29 | 0.08 |
| | 2546* | | | | | | | | | |
| ChemDiv6 | G856- | S | C | 5.08 | 0.04233423 | 0.21 | 0.17 | 0.19 | 0.34 | 0.79 |
| | 2906 | | | | | | | | | |
| ChemDiv6 | G856- | M | C | 4.45 | 0.69035498 | 0.26 | 0.24 | 0.25 | 0.99 | 1.54 |
| | 7017 | | | | | | | | | |
| ChemDiv6 | G856- | W | C | 3.60 | 0.51134253 | 0.22 | 0.19 | 0.20 | −0.03 | 0.28 |
| | 7047 | | | | | | | | | |
| ChemDiv6 | G857- | M | C | 4.61 | 0.3420855 | 0.21 | 0.16 | 0.19 | 0.48 | 0.54 |
| | 0804 | | | | | | | | | |
| ChemDiv6 | K405- | W | C | 3.33 | 0.27103715 | 0.10 | 0.13 | 0.11 | −0.45 | 0.06 |
| | 2989 | | | | | | | | | |
| ChemDiv6 | K405- | M | C | 4.56 | 1.49926444 | 0.16 | 0.14 | 0.15 | 0.67 | 0.16 |
| | 3019 | | | | | | | | | |
| ChemDiv6 | K405- | W | C | 3.59 | 0.39828488 | 0.09 | 0.16 | 0.13 | 0.74 | 0.21 |
| | 3134 | | | | | | | | | |
| ChemDiv6 | K405- | W | C | 3.51 | 0.01118508 | 0.10 | 0.14 | 0.12 | 0.18 | 1.23 |
| | 3259 | | | | | | | | | |
| ChemDiv6 | K405- | M | C | 3.72 | 0.80373402 | 0.12 | 0.13 | 0.13 | 0.08 | 0.22 |
| | 3623 | | | | | | | | | |
| ChemDiv6 | E744- | M | C | 4.13 | 1.24478161 | 0.16 | 0.18 | 0.17 | −0.05 | 0.68 |
| | 0009 | | | | | | | | | |
| ChemDiv7 | G650- | S | C | 11.65 | 1.0126894 | 0.19 | 0.23 | 0.21 | −0.74 | 0.77 |
| | 0193 | | | | | | | | | |
| ChemDiv7 | G748- | W | C | 3.30 | 0.26607886 | 0.04 | 0.04 | 0.04 | −1.11 | 0.35 |
| | 0016 | | | | | | | | | |
| ChemDiv7 | G769- | S | C | 6.67 | 2.32053685 | 0.06 | 0.08 | 0.07 | 0.00 | 0.09 |
| | 1003 | | | | | | | | | |
| ChemDiv7 | G771- | M | C | 4.78 | 2.18555422 | 0.04 | 0.06 | 0.05 | −2.27 | 2.88 |
| | 0448 | | | | | | | | | |
| ChemDiv7 | G771- | M | C | 5.35 | 1.70234917 | 0.05 | 0.06 | 0.05 | 2.05 | 0.25 |
| | 1118 | | | | | | | | | |
| ChemDiv7 | G775- | M | C | 4.95 | 2.31894549 | 0.04 | 0.06 | 0.05 | −1.81 | 1.03 |
| | 0268 | | | | | | | | | |
| ChemDiv7 | G775- | W | C | 3.57 | 0.16786956 | 0.04 | 0.03 | 0.04 | 0.57 | 1.40 |
| | 0671 | | | | | | | | | |
| ChemDiv7 | G786- | M | C | 3.71 | 1.00403109 | 0.03 | 0.04 | 0.04 | −0.41 | 0.85 |
| | 1316 | | | | | | | | | |
| ChemDiv7 | G786- | M | C | 4.76 | 1.74016241 | 0.04 | 0.06 | 0.05 | 0.89 | 2.90 |
| | 1483 | | | | | | | | | |
| ChemDiv7 | G786- | M | C | 6.00 | 2.75669708 | 0.05 | 0.07 | 0.06 | 0.31 | 0.21 |
| | 1562 | | | | | | | | | |
| ChemDiv7 | G786- | M | C | 4.68 | 2.32849661 | 0.03 | 0.06 | 0.05 | 0.37 | 1.53 |
| | 1604 | | | | | | | | | |
| ChemDiv7 | G769- | S | C | 6.31 | 1.53109006 | 0.06 | 0.07 | 0.06 | 1.33 | 1.11 |
| | 1010 | | | | | | | | | |
| ChemDiv7 | G774- | M | C | 3.64 | 0.61572437 | 0.04 | 0.04 | 0.04 | 0.10 | 0.62 |
| | 0218 | | | | | | | | | |
| ChemDiv7 | G775- | M | C | 3.99 | 0.76648867 | 0.04 | 0.04 | 0.04 | 0.12 | 1.10 |
| | 0475 | | | | | | | | | |
| ChemDiv7 | G775- | W | C | 3.06 | 0.00583024 | 0.03 | 0.03 | 0.03 | −0.22 | 1.45 |
| | 0674 | | | | | | | | | |
| ChemDiv7 | G786- | M | C | 4.32 | 0.96476599 | 0.04 | 0.05 | 0.04 | 1.23 | 0.10 |
| | 0334 | | | | | | | | | |
| ChemDiv7 | G786- | M | C | 4.72 | 1.7326314 | 0.04 | 0.06 | 0.05 | 0.33 | 1.60 |
| | 1317 | | | | | | | | | |
| ChemDiv7 | G786- | M | C | 4.73 | 0.46367575 | 0.06 | 0.04 | 0.05 | −0.24 | 0.17 |
| | 1567 | | | | | | | | | |
| ChemDiv7 | G769- | M | C | 5.47 | 1.75334447 | 0.05 | 0.06 | 0.05 | 0.14 | 0.23 |
| | 1017 | | | | | | | | | |
| ChemDiv7 | G775- | M | C | 3.89 | 0.51374231 | 0.04 | 0.04 | 0.04 | 0.10 | 0.70 |
| | 0507 | | | | | | | | | |
| ChemDiv7 | G786- | M | | 4.57 | 0.83972267 | 0.04 | 0.05 | 0.05 | 0.40 | 1.21 |
| | 0268* | | | | | | | | | |
| ChemDiv7 | G786- | M | C | 5.09 | 1.30404791 | 0.05 | 0.06 | 0.05 | 0.03 | 0.27 |
| | 1254 | | | | | | | | | |
| ChemDiv7 | G786- | M | C | 4.14 | 0.92226264 | 0.04 | 0.04 | 0.04 | 0.93 | 1.57 |
| | 0335 | | | | | | | | | |
| ChemDiv7 | G786- | M | C | 4.44 | 0.18843782 | 0.05 | 0.04 | 0.05 | 0.61 | 0.83 |
| | 1324 | | | | | | | | | |
| ChemDiv7 | G786- | M | C | 4.14 | 1.38315312 | 0.04 | 0.05 | 0.04 | 0.05 | 0.42 |
| | 1534 | | | | | | | | | |
| ChemDiv7 | G786- | M | C | 6.77 | 4.41045384 | 0.04 | 0.09 | 0.07 | −0.37 | 0.82 |
| | 1569 | | | | | | | | | |
| ChemDiv7 | G774- | W | C | 3.44 | 0.32121283 | 0.04 | 0.03 | 0.04 | 0.27 | 2.04 |
| | 0231 | | | | | | | | | |
| ChemDiv7 | G786- | M | C | 4.41 | 0.23445033 | 0.05 | 0.04 | 0.05 | 0.56 | 0.30 |
| | 0269 | | | | | | | | | |
| ChemDiv7 | G786- | M | C | 4.35 | 0.46661134 | 0.05 | 0.04 | 0.04 | 0.49 | 0.76 |
| | 1325 | | | | | | | | | |
| ChemDiv7 | G786- | M | C | 4.46 | 1.42716262 | 0.04 | 0.05 | 0.04 | 0.31 | 0.39 |
| | 1537 | | | | | | | | | |
| ChemDiv7 | G786- | M | C | 3.92 | 0.97395245 | 0.04 | 0.04 | 0.04 | 0.31 | 0.66 |
| | 1570 | | | | | | | | | |
| ChemDiv7 | G786- | M | C | 4.64 | 0.13549472 | 0.05 | 0.04 | 0.05 | −1.38 | 0.87 |
| | 1665 | | | | | | | | | |
| ChemDiv7 | G786- | S | C | 8.08 | 1.67274635 | 0.08 | 0.09 | 0.08 | 0.46 | 0.89 |
| | 2334 | | | | | | | | | |
| ChemDiv7 | G769- | M | C | 5.47 | 0.96576494 | 0.05 | 0.06 | 0.06 | 1.62 | 0.45 |
| | 1036 | | | | | | | | | |
| ChemDiv7 | G784- | S | C | 7.48 | 2.88756106 | 0.06 | 0.09 | 0.07 | −2.11 | 1.10 |
| | 0099 | | | | | | | | | |
| ChemDiv7 | G775- | W | C | 3.57 | 0.39036557 | 0.04 | 0.03 | 0.04 | 0.15 | 0.87 |
| | 0370 | | | | | | | | | |
| ChemDiv7 | G786- | W | C | 3.65 | 0.40357015 | 0.04 | 0.04 | 0.04 | 0.48 | 1.11 |
| | 0272 | | | | | | | | | |
| ChemDiv7 | G786- | W | C | 3.92 | 0.07665304 | 0.04 | 0.04 | 0.04 | 1.54 | 0.44 |
| | 1471 | | | | | | | | | |
| ChemDiv7 | G786- | W | C | 3.81 | 0.07112701 | 0.04 | 0.04 | 0.04 | −0.38 | 0.06 |
| | 1547 | | | | | | | | | |
| ChemDiv7 | G786- | W | C | 3.86 | 0.00419909 | 0.04 | 0.04 | 0.04 | 1.96 | 1.41 |
| | 1572 | | | | | | | | | |
| ChemDiv7 | G786- | W | C | 3.56 | 0.37653036 | 0.04 | 0.04 | 0.04 | 0.90 | 2.32 |
| | 2003 | | | | | | | | | |
| ChemDiv7 | G771- | M | C | 4.55 | 0.26383445 | 0.05 | 0.04 | 0.05 | −1.34 | 1.63 |
| | 0002 | | | | | | | | | |
| ChemDiv7 | G786- | M | C | 3.87 | 0.44607174 | 0.04 | 0.04 | 0.04 | −2.94 | 1.04 |
| | 0273 | | | | | | | | | |
| ChemDiv7 | G786- | M | C | 3.68 | 0.91950468 | 0.03 | 0.04 | 0.04 | 0.70 | 0.19 |
| | 1263 | | | | | | | | | |
| ChemDiv7 | G786- | W | C | 3.69 | 0.34259594 | 0.04 | 0.04 | 0.04 | −0.30 | 1.10 |
| | 1551 | | | | | | | | | |
| ChemDiv7 | G771- | S | C | 6.44 | 0.64687477 | 0.07 | 0.06 | 0.07 | −0.15 | 0.67 |
| | 0008 | | | | | | | | | |
| ChemDiv7 | G771- | M | C | 4.85 | 0.25946824 | 0.05 | 0.05 | 0.05 | −1.58 | 1.20 |
| | 0699 | | | | | | | | | |
| ChemDiv7 | G775- | W | C | 3.76 | 0.00746652 | 0.04 | 0.03 | 0.04 | −0.13 | 0.49 |
| | 0401 | | | | | | | | | |
| ChemDiv7 | G775- | M | C | 4.33 | 1.58578957 | 0.04 | 0.05 | 0.04 | −0.28 | 0.60 |
| | 0641 | | | | | | | | | |
| ChemDiv7 | G786- | M | C | 4.35 | 1.14777447 | 0.04 | 0.05 | 0.04 | 2.19 | 0.90 |
| | 0299 | | | | | | | | | |
| ChemDiv7 | G786- | S | C | 5.95 | 0.68175095 | 0.06 | 0.06 | 0.06 | 1.35 | 2.24 |
| | 1264 | | | | | | | | | |
| ChemDiv7 | G786- | W | C | 3.54 | 0.36276235 | 0.04 | 0.04 | 0.04 | 0.40 | 1.51 |
| | 0344 | | | | | | | | | |
| ChemDiv7 | G786- | M | C | 5.26 | 0.88794552 | 0.05 | 0.05 | 0.05 | 0.68 | 1.81 |
| | 1481 | | | | | | | | | |
| ChemDiv7 | G786- | M | C | 4.68 | 0.59009539 | 0.06 | 0.04 | 0.05 | −0.50 | 0.28 |
| | 1559 | | | | | | | | | |
| ChemDiv7 | G786- | M | C | 4.61 | 0.57848083 | 0.05 | 0.05 | 0.05 | 0.86 | 0.11 |
| | 1588 | | | | | | | | | |
| ChemDiv7 | G786- | M | C | 4.01 | 1.26545609 | 0.06 | 0.03 | 0.04 | −0.09 | 0.95 |
| | 1669 | | | | | | | | | |
| ChemDiv7 | G786- | W | C | 3.18 | 0.07773524 | 0.04 | 0.03 | 0.03 | 0.75 | 0.40 |
| | 1822 | | | | | | | | | |
| ChemDiv7 | G771- | M | C | 4.95 | 1.46582405 | 0.04 | 0.06 | 0.05 | 0.10 | 0.10 |
| | 0015 | | | | | | | | | |
| ChemDiv7 | G771- | W | C | 3.27 | 0.01173593 | 0.04 | 0.03 | 0.03 | 1.18 | 0.91 |
| | 0374 | | | | | | | | | |
| ChemDiv7 | G771- | W | C | 3.91 | 0.080451 | 0.04 | 0.04 | 0.04 | −0.87 | 0.28 |
| | 0900 | | | | | | | | | |
| ChemDiv7 | G784- | M | C | 3.61 | 0.71834458 | 0.03 | 0.04 | 0.04 | 0.32 | 1.02 |
| | 0087 | | | | | | | | | |
| ChemDiv7 | G784- | M | C | 3.89 | 0.40896447 | 0.05 | 0.03 | 0.04 | −0.83 | 0.73 |
| | 0129 | | | | | | | | | |
| ChemDiv7 | G775- | W | C | 3.39 | 0.21734986 | 0.04 | 0.03 | 0.03 | 0.22 | 2.49 |
| | 0454 | | | | | | | | | |
| ChemDiv7 | G779- | W | C | 3.73 | 0.17529827 | 0.04 | 0.04 | 0.04 | 1.45 | 0.47 |
| | 0144 | | | | | | | | | |
| ChemDiv7 | G786- | M | C | 4.28 | 0.6604661 | 0.05 | 0.04 | 0.04 | −0.06 | 0.57 |
| | 0317 | | | | | | | | | |
| ChemDiv7 | G786- | M | C | 4.92 | 0.80229003 | 0.05 | 0.05 | 0.05 | −0.93 | 0.29 |
| | 1280 | | | | | | | | | |
| ChemDiv7 | G786- | M | C | 3.99 | 1.08531621 | 0.05 | 0.03 | 0.04 | 1.06 | 1.06 |
| | 0351 | | | | | | | | | |
| ChemDiv7 | G786- | S | C | 6.09 | 0.64120956 | 0.06 | 0.06 | 0.06 | 0.30 | 0.48 |
| | 1482 | | | | | | | | | |
| ChemDiv7 | G786- | M | C | 3.92 | 1.22800428 | 0.05 | 0.03 | 0.04 | −0.16 | 0.38 |
| | 0389 | | | | | | | | | |
| ChemDiv7 | G786- | S | C | 5.20 | 0.15478961 | 0.06 | 0.05 | 0.05 | −1.01 | 0.92 |
| | 1561 | | | | | | | | | |
| ChemDiv7 | G786- | M | C | 4.73 | 0.03662143 | 0.05 | 0.04 | 0.05 | −0.71 | 1.62 |
| | 0400 | | | | | | | | | |
| ChemDiv7 | G786- | M | C | 4.52 | 0.04603241 | 0.05 | 0.04 | 0.05 | −0.38 | 0.75 |
| | 1602 | | | | | | | | | |
| ChemDiv7 | G786- | M | C | 4.11 | 0.7152355 | 0.04 | 0.04 | 0.04 | −2.12 | 3.00 |
| | 0423 | | | | | | | | | |
| ChemDiv7 | G786- | M | C | 4.58 | 0.22789764 | 0.05 | 0.04 | 0.05 | −0.68 | 0.50 |
| | 1670 | | | | | | | | | |
| ChemDiv7 | G786- | M | C | 3.87 | 0.30473905 | 0.04 | 0.04 | 0.04 | 0.72 | 0.08 |
| | 2194 | | | | | | | | | |
| ChemDiv7 | G786- | W | C | 3.64 | 0.24279943 | 0.04 | 0.03 | 0.04 | 1.75 | 0.66 |
| | 2325 | | | | | | | | | |
| ChemDiv7 | G856- | M | C | 3.73 | 0.57850597 | 0.05 | 0.07 | 0.06 | −1.06 | 0.16 |
| | 9836 | | | | | | | | | |
| ChemDiv7 | G881- | M | C | 5.83 | 3.79301448 | 0.15 | 0.08 | 0.12 | −0.10 | 0.08 |
| | 0290 | | | | | | | | | |
| ChemDiv7 | G881- | M | C | 4.85 | 2.56595748 | 0.12 | 0.08 | 0.10 | 0.08 | 1.11 |
| | 0295 | | | | | | | | | |
| ChemDiv7 | G881- | M | C | 5.65 | 1.09712758 | 0.12 | 0.12 | 0.12 | −0.75 | 0.73 |
| | 0499 | | | | | | | | | |
| ChemDiv7 | G889- | S | | 7.46 | 2.26739286 | 0.18 | 0.13 | 0.15 | 1.59 | 0.28 |
| | 0412* | | | | | | | | | |
| ChemDiv7 | G889- | W | | 3.38 | 0.08064893 | 0.07 | 0.07 | 0.07 | 0.17 | 0.08 |
| | 0171* | | | | | | | | | |
| ChemDiv7 | G933- | M | C | 4.30 | 0.08820626 | 0.07 | 0.07 | 0.07 | −0.65 | 0.10 |
| | 0058 | | | | | | | | | |
| ChemDiv7 | G935- | W | C | 3.23 | 0.06791224 | 0.05 | 0.06 | 0.05 | −0.95 | 0.71 |
| | 2190 | | | | | | | | | |
| ChemDiv7 | G946- | M | C | 3.52 | 0.67409894 | 0.05 | 0.07 | 0.06 | 0.15 | 0.48 |
| | 0483 | | | | | | | | | |
| ChemDiv7 | G947- | M | C | 4.04 | 0.3462132 | 0.06 | 0.07 | 0.07 | 0.53 | 0.45 |
| | 0023 | | | | | | | | | |
| ChemDiv7 | G947- | W | C | 3.61 | 0.42342254 | 0.05 | 0.07 | 0.06 | −0.39 | 0.77 |
| | 4440 | | | | | | | | | |
| ChemDiv7 | G948- | W | C | 3.42 | 0.40619629 | 0.06 | 0.05 | 0.06 | −0.59 | 0.60 |
| | 5129 | | | | | | | | | |
| ChemDiv7 | G933- | M | C | 3.85 | 0.35740401 | 0.07 | 0.06 | 0.06 | −0.15 | 0.25 |
| | 0087 | | | | | | | | | |
| ChemDiv7 | G935- | W | C | 3.49 | 0.1374264 | 0.06 | 0.06 | 0.06 | −0.20 | 0.51 |
| | 2193 | | | | | | | | | |
| ChemDiv7 | G937- | W | C | 3.15 | 0.17583577 | 0.05 | 0.05 | 0.05 | 0.53 | 0.18 |
| | 0468 | | | | | | | | | |
| ChemDiv7 | G946- | M | C | 3.72 | 0.39649291 | 0.06 | 0.06 | 0.06 | −0.52 | 1.37 |
| | 0405 | | | | | | | | | |
| ChemDiv7 | G946- | W | C | 3.58 | 0.08129683 | 0.06 | 0.06 | 0.06 | −0.88 | 0.57 |
| | 0488 | | | | | | | | | |
| ChemDiv7 | G946- | W | C | 3.54 | 0.10337432 | 0.06 | 0.06 | 0.06 | 0.53 | 0.08 |
| | 0601 | | | | | | | | | |
| ChemDiv7 | G947- | W | C | 3.94 | 0.03381916 | 0.06 | 0.07 | 0.07 | −0.77 | 0.79 |
| | 0077 | | | | | | | | | |
| ChemDiv7 | G947- | W | C | 3.40 | 0.41816827 | 0.05 | 0.06 | 0.06 | −0.12 | 0.72 |
| | 4580 | | | | | | | | | |
| ChemDiv7 | J006- | W | C | 3.57 | 0.43541362 | 0.05 | 0.05 | 0.05 | −0.21 | 0.28 |
| | 0457 | | | | | | | | | |
| ChemDiv7 | J015- | M | C | 3.83 | 1.17266426 | 0.06 | 0.04 | 0.05 | 0.15 | 0.16 |
| | 0213 | | | | | | | | | |
| ChemDiv7 | J026- | M | C | 4.03 | 0.71594781 | 0.04 | 0.06 | 0.05 | −0.81 | 0.02 |
| | 0004 | | | | | | | | | |
| ChemDiv7 | J015- | W | C | 3.63 | 0.04193843 | 0.04 | 0.04 | 0.04 | −0.61 | 0.09 |
| | 0222 | | | | | | | | | |
| ChemDiv7 | J015- | M | C | 4.17 | 1.24637271 | 0.04 | 0.06 | 0.05 | −0.52 | 0.18 |
| | 0225 | | | | | | | | | |
| ChemDiv7 | J021- | M | C | 4.13 | 0.44312753 | 0.05 | 0.05 | 0.05 | 1.45 | 0.02 |
| | 0103 | | | | | | | | | |
| ChemDiv7 | J024- | W | C | 3.44 | 0.01637494 | 0.04 | 0.04 | 0.04 | 0.84 | 0.10 |
| | 0550 | | | | | | | | | |
| ChemDiv7 | J026- | S | C | 10.84 | 5.72677565 | 0.18 | 0.08 | 0.13 | −1.01 | 0.07 |
| | 0217 | | | | | | | | | |
| ChemDiv7 | J015- | W | C | 3.72 | 0.24730125 | 0.04 | 0.05 | 0.04 | −0.50 | 0.17 |
| | 0243 | | | | | | | | | |
| ChemDiv7 | J024- | M | C | 3.83 | 0.61132635 | 0.05 | 0.04 | 0.05 | −0.85 | 0.05 |
| | 0623 | | | | | | | | | |
| ChemDiv7 | J024- | M | C | 3.68 | 0.52226718 | 0.05 | 0.04 | 0.04 | −1.07 | 0.11 |
| | 0800 | | | | | | | | | |
| ChemDiv7 | J026- | M | C | 4.40 | 0.13017541 | 0.05 | 0.05 | 0.05 | −0.33 | 0.19 |
| | 3874 | | | | | | | | | |
| ChemDiv7 | J030- | W | C | 3.70 | 0.41334199 | 0.06 | 0.05 | 0.05 | 0.49 | 0.30 |
| | 0068 | | | | | | | | | |
| ChemDiv7 | J030- | M | C | 4.58 | 0.1831547 | 0.06 | 0.07 | 0.06 | 0.40 | 0.90 |
| | 0069 | | | | | | | | | |
| ChemDiv7 | J065- | M | C | 5.05 | 2.12438887 | 0.10 | 0.14 | 0.12 | −0.30 | 0.05 |
| | 2258 | | | | | | | | | |
| ChemDiv7 | J075- | W | C | 3.43 | 0.33662153 | 0.09 | 0.08 | 0.09 | 0.88 | 2.56 |
| | 3081 | | | | | | | | | |
| ChemDiv7 | 1080- | W | C | 3.76 | 0.28976082 | 0.10 | 0.08 | 0.09 | 1.63 | 0.43 |
| | 0666 | | | | | | | | | |
| ChemDiv7 | J075- | W | C | 3.01 | 0.0221053 | 0.09 | 0.06 | 0.08 | 0.50 | 2.14 |
| | 2706 | | | | | | | | | |
| ChemDiv7 | 1094- | M | C | 4.94 | 0.3158556 | 0.15 | 0.15 | 0.15 | −0.31 | 1.40 |
| | 0187 | | | | | | | | | |
| ChemDiv7 | K261- | M | C | 4.25 | 0.16569456 | 0.15 | 0.17 | 0.16 | 0.67 | 0.73 |
| | 1443 | | | | | | | | | |
| ChemDiv7 | L063- | M | C | 3.94 | 1.29618282 | 0.10 | 0.07 | 0.08 | −0.23 | 0.34 |
| | 0010 | | | | | | | | | |
| ChemDiv7 | L065- | W | C | 3.59 | 0.1979135 | 0.07 | 0.09 | 0.08 | −0.34 | 0.09 |
| | 0741 | | | | | | | | | |
| ChemDiv7 | L262- | S | C | 7.71 | 1.75016956 | 0.22 | 0.21 | 0.22 | −0.11 | 0.93 |
| | 0843 | | | | | | | | | |
| ChemDiv7 | L378- | M | C | 4.37 | 1.76986843 | 0.12 | 0.17 | 0.15 | −0.93 | 0.11 |
| | 0328 | | | | | | | | | |
| ChemDiv7 | L378- | M | C | 4.60 | 0.36163943 | 0.19 | 0.14 | 0.16 | −0.74 | 0.28 |
| | 0329 | | | | | | | | | |
| ChemDiv7 | L378- | M | C | 5.44 | 1.97268176 | 0.16 | 0.21 | 0.19 | −0.84 | 1.20 |
| | 0331 | | | | | | | | | |
| ChemDiv7 | L538- | M | C | 4.95 | 2.38891281 | 0.23 | 0.13 | 0.18 | −1.53 | 0.84 |
| | 0006 | | | | | | | | | |
| ChemDiv7 | L538- | M | C | 4.72 | 0.01043022 | 0.17 | 0.19 | 0.18 | −0.77 | 0.63 |
| | 0010 | | | | | | | | | |
| ChemDiv7 | L662- | S | C | 5.59 | 0.73226157 | 0.27 | 0.31 | 0.29 | −0.68 | 0.59 |
| | 0607 | | | | | | | | | |
| ChemDiv7 | L662- | W | C | 3.75 | 0.20893696 | 0.19 | 0.20 | 0.19 | −0.99 | 0.08 |
| | 0688 | | | | | | | | | |
| ChemDiv7 | L663- | M | C | 5.18 | 1.482525 | 0.22 | 0.31 | 0.27 | −0.82 | 0.29 |
| | 1002 | | | | | | | | | |
| ChemDiv7 | L662- | M | C | 4.86 | 0.86666069 | 0.23 | 0.27 | 0.25 | −0.73 | 0.19 |
| | 0660 | | | | | | | | | |
| ChemDiv7 | L663- | S | C | 7.72 | 0.76022395 | 0.38 | 0.41 | 0.40 | −0.69 | 0.08 |
| | 1076 | | | | | | | | | |
| ChemDiv7 | L662- | M | C | 3.56 | 0.67247408 | 0.21 | 0.15 | 0.18 | 0.56 | 0.86 |
| | 0668 | | | | | | | | | |
| ChemDiv7 | L662- | S | C | 5.13 | 0.17940927 | 0.27 | 0.26 | 0.26 | −0.92 | 0.28 |
| | 0692 | | | | | | | | | |
| ChemDiv7 | L662- | M | C | 4.06 | 0.48434618 | 0.20 | 0.22 | 0.21 | −0.90 | 0.13 |
| | 0345 | | | | | | | | | |
| ChemDiv7 | L662- | S | C | 5.26 | 0.12036839 | 0.28 | 0.27 | 0.27 | −0.96 | 0.18 |
| | 0693 | | | | | | | | | |
| ChemDiv7 | L662- | M | | 3.92 | 0.45306768 | 0.23 | 0.18 | 0.20 | −0.27 | 0.15 |
| | 0639* | | | | | | | | | |
| ChemDiv7 | L662- | M | C | 5.63 | 1.21957666 | 0.35 | 0.24 | 0.29 | −0.75 | 0.50 |
| | 0973 | | | | | | | | | |
| ChemDiv7 | L662- | M | C | 5.27 | 0.67508962 | 0.25 | 0.29 | 0.27 | −0.79 | 0.29 |
| | 0315 | | | | | | | | | |
| ChemDiv7 | L662- | M | C | 4.87 | 0.0584357 | 0.26 | 0.24 | 0.25 | −0.96 | 0.17 |
| | 0678 | | | | | | | | | |
| ChemDiv7 | L662- | S | C | 8.64 | 1.00731695 | 0.42 | 0.47 | 0.44 | −1.09 | 0.11 |
| | 0977 | | | | | | | | | |
| ChemDiv7 | L663- | S | C | 5.51 | 0.3848383 | 0.28 | 0.29 | 0.28 | −0.76 | 0.64 |
| | 1062 | | | | | | | | | |
| ChemDiv7 | L662- | S | C | 7.15 | 0.48314565 | 0.36 | 0.37 | 0.37 | −0.76 | 0.50 |
| | 0597 | | | | | | | | | |
| ChemDiv7 | L662- | S | C | 5.79 | 0.11636146 | 0.30 | 0.29 | 0.30 | −0.62 | 0.48 |
| | 0654 | | | | | | | | | |
| ChemDiv7 | L662- | S | C | 7.18 | 0.06140255 | 0.38 | 0.36 | 0.37 | −0.48 | 0.58 |
| | 0987 | | | | | | | | | |
| ChemDiv7 | L663- | S | C | 7.57 | 0.69624117 | 0.43 | 0.35 | 0.39 | −1.03 | 0.13 |
| | 0996 | | | | | | | | | |
| ChemDiv7 | L663- | W | C | 3.27 | 0.21768365 | 0.17 | 0.17 | 0.17 | −0.69 | 0.74 |
| | 1064 | | | | | | | | | |
| ChemDiv7 | L662- | M | C | 4.71 | 0.15627293 | 0.24 | 0.24 | 0.24 | −0.55 | 0.08 |
| | 0325 | | | | | | | | | |
| ChemDiv7 | L662- | S | C | 6.87 | 0.9600493 | 0.33 | 0.38 | 0.35 | −1.17 | 0.22 |
| | 0604 | | | | | | | | | |
| ChemDiv7 | L662- | M | C | 4.71 | 0.29530624 | 0.26 | 0.23 | 0.24 | 0.91 | 0.70 |
| | 0655 | | | | | | | | | |
| ChemDiv7 | L662- | W | C | 3.53 | 0.28974825 | 0.18 | 0.19 | 0.18 | −0.96 | 0.37 |
| | 0686 | | | | | | | | | |
| ChemDiv7 | L663- | S | C | 7.77 | 0.81870865 | 0.44 | 0.36 | 0.40 | −0.73 | 0.38 |
| | 0999 | | | | | | | | | |
| ChemDiv7 | L921- | S | C | 5.32 | 0.04842893 | 0.08 | 0.08 | 0.08 | 0.39 | 0.63 |
| | 1051 | | | | | | | | | |
| ChemDiv7 | L921- | S | C | 11.75 | 2.41848685 | 0.19 | 0.15 | 0.17 | 0.07 | 0.51 |
| | 1004 | | | | | | | | | |
| ChemDiv7 | L942- | M | C | 3.89 | 0.62118422 | 0.05 | 0.07 | 0.06 | −1.04 | 0.27 |
| | 0265 | | | | | | | | | |
| ChemDiv7 | L921- | S | C | 6.52 | 0.07507106 | 0.09 | 0.10 | 0.10 | −0.01 | 0.21 |
| | 0997 | | | | | | | | | |
| ChemDiv7 | L977- | M | | 4.14 | 0.96232842 | 0.06 | 0.08 | 0.07 | −0.67 | 0.56 |
| | 1300* | | | | | | | | | |
| ChemDiv7 | L977- | S | | 8.42 | 0.23181768 | 0.15 | 0.13 | 0.14 | 0.02 | 0.78 |
| | 0017* | | | | | | | | | |
| ChemDiv7 | M040- | M | C | 5.52 | 1.71158348 | 0.11 | 0.05 | 0.08 | 0.81 | 0.32 |
| | 0093 | | | | | | | | | |
| ChemDiv7 | M040- | M | C | 5.08 | 0.42573469 | 0.08 | 0.07 | 0.07 | 1.37 | 0.40 |
| | 0082 | | | | | | | | | |
| ChemDiv7 | M040- | S | C | 7.25 | 0.82229514 | 0.11 | 0.10 | 0.11 | 2.81 | 0.10 |
| | 0094 | | | | | | | | | |
| ChemDiv7 | M040- | M | C | 5.83 | 1.53286609 | 0.08 | 0.09 | 0.08 | 0.62 | 0.49 |
| | 0159 | | | | | | | | | |
| ChemDiv7 | M040- | S | C | 5.98 | 0.4521693 | 0.11 | 0.07 | 0.09 | 1.93 | 0.26 |
| | 0428 | | | | | | | | | |
| ChemDiv7 | M040- | S | C | 6.62 | 0.94333292 | 0.10 | 0.09 | 0.10 | 2.90 | 0.03 |
| | 0083 | | | | | | | | | |
| ChemDiv7 | M040- | S | C | 7.07 | 0.14909477 | 0.12 | 0.09 | 0.10 | 2.25 | 0.13 |
| | 0939 | | | | | | | | | |
| ChemDiv7 | M040- | M | C | 4.42 | 0.93795626 | 0.09 | 0.05 | 0.07 | 1.94 | 0.44 |
| | 0084 | | | | | | | | | |
| ChemDiv7 | M040- | M | C | 3.68 | 0.9080654 | 0.07 | 0.04 | 0.06 | 1.50 | 0.70 |
| | 0170 | | | | | | | | | |
| ChemDiv7 | M040- | S | C | 6.31 | 0.17451836 | 0.10 | 0.08 | 0.09 | 2.21 | 0.10 |
| | 0194 | | | | | | | | | |
| ChemDiv7 | M040- | M | C | 4.78 | 1.67712678 | 0.10 | 0.05 | 0.07 | 2.23 | 0.82 |
| | 0116 | | | | | | | | | |
| ChemDiv7 | M040- | M | C | 3.71 | 0.51441947 | 0.07 | 0.04 | 0.06 | 1.48 | 1.08 |
| | 0039 | | | | | | | | | |
| ChemDiv7 | M040- | M | C | 4.68 | 0.67014325 | 0.09 | 0.05 | 0.07 | 1.23 | 0.30 |
| | 0079 | | | | | | | | | |
| ChemDiv7 | M040- | S | C | 7.27 | 0.07205363 | 0.12 | 0.09 | 0.11 | 1.82 | 0.16 |
| | 0118 | | | | | | | | | |
| ChemDiv7 | M040- | S | C | 7.78 | 0.35593062 | 0.13 | 0.10 | 0.11 | 2.89 | 0.00 |
| | 0203 | | | | | | | | | |
| ChemDiv7 | M060- | S | C | 9.15 | 1.43292014 | 0.12 | 0.11 | 0.12 | 1.27 | 0.20 |
| | 0419 | | | | | | | | | |
| ChemDiv7 | M056- | M | | 4.82 | 2.0043562 | 0.05 | 0.07 | 0.06 | −0.92 | 0.13 |
| | 0617* | | | | | | | | | |
| ChemDiv7 | M115- | M | C | 5.58 | 2.48485654 | 0.10 | 0.07 | 0.08 | −0.47 | 0.52 |
| | 0551 | | | | | | | | | |
| ChemDiv7 | M184- | M | C | 5.32 | 1.05814732 | 0.07 | 0.07 | 0.07 | −0.68 | 0.25 |
| | 0111 | | | | | | | | | |
| ChemDiv7 | M144- | W | C | 3.45 | 0.4741242 | 0.04 | 0.06 | 0.05 | 2.64 | 0.03 |
| | 0886 | | | | | | | | | |
| ChemDiv7 | M130- | S | C | 20.61 | 3.76126669 | 0.28 | 0.28 | 0.28 | −1.21 | 0.22 |
| | 0012 | | | | | | | | | |
| ChemDiv7 | P615- | M | C | 6.30 | 2.7370921 | 0.08 | 0.06 | 0.07 | −0.94 | 0.35 |
| | 0091 | | | | | | | | | |
| ChemDiv7 | P563- | W | | 3.27 | 0.35264029 | 0.04 | 0.04 | 0.04 | 1.59 | 0.22 |
| | 1219* | | | | | | | | | |
| ChemDiv7 | P616- | M | C | 4.18 | 0.62292323 | 0.04 | 0.06 | 0.05 | 0.44 | 1.97 |
| | 0072 | | | | | | | | | |
| ChemDiv7 | P773- | M | C | 4.00 | 0.04637968 | 0.06 | 0.05 | 0.05 | −1.04 | 1.65 |
| | 6557 | | | | | | | | | |
| ChemDiv7 | T828- | M | C | 3.67 | 0.87788771 | 0.05 | 0.04 | 0.05 | −0.65 | 0.41 |
| | 0650 | | | | | | | | | |
| Enamine2 | T5324509 | S | C | 7.80 | 2.84437753 | 0.12 | 0.09 | 0.10 | −0.73 | 0.56 |
| Enamine2 | T5272320 | S | C | 6.46 | 1.01160244 | 0.08 | 0.11 | 0.09 | 1.24 | 0.55 |
| Enamine2 | T5277337 | M | C | 4.76 | 1.08796545 | 0.07 | 0.06 | 0.07 | 1.91 | 0.90 |
| Enamine2 | T5253378* | M | | 4.20 | 0.1051916 | 0.08 | 0.07 | 0.07 | −0.33 | 0.42 |
| Enamine2 | T5312445 | S | C | 8.72 | 1.14008636 | 0.12 | 0.11 | 0.11 | 1.54 | 0.61 |
| Enamine2 | T5264279 | M | C | 3.87 | 0.81990218 | 0.06 | 0.08 | 0.07 | −0.67 | 0.37 |
| Enamine2 | T5297621 | M | C | 4.27 | 0.84943775 | 0.05 | 0.07 | 0.06 | 0.96 | 0.30 |
| Enamine2 | T0505- | W | C | 3.59 | 0.02479958 | 0.05 | 0.05 | 0.05 | 0.83 | 0.05 |
| | 0639 | | | | | | | | | |
| Enamine2 | T5279952 | S | C | 7.14 | 1.8223868 | 0.08 | 0.12 | 0.10 | −0.61 | 0.07 |
| Enamine2 | T5281519 | W | C | 3.55 | 0.2679082 | 0.05 | 0.05 | 0.05 | 2.00 | 0.01 |
| Enamine2 | T0504- | M | | 4.97 | 0.79463339 | 0.08 | 0.06 | 0.07 | 2.00 | 0.10 |
| | 9577* | | | | | | | | | |
| Enamine2 | T0505- | M | C | 3.79 | 0.87026952 | 0.06 | 0.05 | 0.05 | −0.68 | 0.83 |
| | 1471 | | | | | | | | | |
| Enamine2 | T5275734 | M | C | 3.99 | 0.13383317 | 0.06 | 0.06 | 0.06 | −0.01 | 0.05 |
| Enamine2 | T0503- | W | C | 3.32 | 0.30221666 | 0.06 | 0.04 | 0.05 | 1.51 | 0.26 |
| | 2749 | | | | | | | | | |
| Enamine2 | T5407333 | M | C | 4.88 | 0.5391335 | 0.09 | 0.08 | 0.08 | −1.39 | 2.43 |
| | 7 | | | | | | | | | |
| Enamine2 | T546307 | M | C | 4.27 | 0.47623614 | 0.06 | 0.09 | 0.08 | 1.42 | 0.85 |
| Enamine2 | T5462423 | W | C | 3.71 | 0.01561832 | 0.06 | 0.07 | 0.06 | 0.18 | 1.40 |
| Enamine2 | T5462389 | W | C | 3.66 | 0.38145102 | 0.05 | 0.08 | 0.06 | −1.17 | 0.88 |
| Enamine2 | T5379816 | W | C | 3.41 | 0.18530819 | 0.05 | 0.06 | 0.06 | 0.01 | 1.26 |
| Enamine2 | T5498737* | W | | 3.34 | 0.1505296 | 0.13 | 0.08 | 0.10 | 1.23 | 0.46 |
| Enamine2 | T5437474 | W | C | 3.13 | 0.18320716 | 0.12 | 0.07 | 0.10 | 0.03 | 1.02 |
| Enamine2 | T5455186 | S | C | 8.58 | 2.42518446 | 0.30 | 0.19 | 0.24 | 0.93 | 0.35 |
| Enamine2 | T5461482 | W | C | 3.23 | 0.1522919 | 0.09 | 0.10 | 0.10 | −0.04 | 0.64 |
| Enamine2 | T5383638 | W | C | 3.56 | 0.26993028 | 0.10 | 0.06 | 0.08 | −0.92 | 0.53 |
| Enamine2 | T5238562 | W | C | 3.42 | 0.22757382 | 0.07 | 0.07 | 0.07 | 0.24 | 0.18 |
| Enamine2 | T0519- | M | C | 5.06 | 1.21984799 | 0.08 | 0.12 | 0.10 | −0.43 | 0.17 |
| | 9012 | | | | | | | | | |
| Enamine2 | T5451097 | M | C | 5.09 | 1.54752826 | 0.14 | 0.20 | 0.17 | 1.78 | 0.74 |
| Enamine2 | T5404600 | M | C | 3.95 | 1.32214221 | 0.12 | 0.10 | 0.11 | −0.80 | 0.26 |
| Enamine2 | T5399241 | M | C | 3.79 | 0.75026201 | 0.11 | 0.10 | 0.11 | 0.10 | 0.58 |
| Enamine2 | T5348278 | W | C | 3.28 | 0.15717372 | 0.08 | 0.10 | 0.09 | −0.08 | 0.82 |
| Enamine2 | T5398430 | M | C | 3.83 | 0.31106171 | 0.10 | 0.11 | 0.11 | 0.05 | 0.52 |
| Enamine2 | T5380152 | M | C | 4.58 | 1.44735385 | 0.14 | 0.11 | 0.13 | −1.03 | 0.09 |
| Enamine2 | T5385382 | S | C | 7.72 | 2.69115117 | 0.10 | 0.12 | 0.11 | 2.35 | 0.11 |
| Enamine2 | T0519- | M | C | 4.54 | 0.27036631 | 0.05 | 0.07 | 0.06 | −0.65 | 0.47 |
| | 6400 | | | | | | | | | |
| Enamine2 | T0503- | S | C | 14.21 | 1.99441281 | 0.16 | 0.16 | 0.16 | −0.71 | 0.22 |
| | 6223 | | | | | | | | | |
| Enamine2 | T5441809 | S | C | 22.96 | 5.84856305 | 0.27 | 0.24 | 0.26 | 1.95 | 0.25 |
| Enamine2 | T5441826 | M | C | 4.20 | 1.17568502 | 0.03 | 0.06 | 0.05 | −0.56 | 0.83 |
| Enamine2 | T0503- | S | C | 6.86 | 0.79311995 | 0.06 | 0.10 | 0.08 | −1.08 | 0.08 |
| | 6911 | | | | | | | | | |
| Enamine2 | T5229649 | M | C | 4.90 | 1.64974268 | 0.06 | 0.08 | 0.07 | −0.42 | 0.09 |
| Enamine2 | T0503- | M | C | 4.67 | 0.0380214 | 0.06 | 0.06 | 0.06 | −0.45 | 0.04 |
| | 9777 | | | | | | | | | |
| Enamine2 | T0504- | S | C | 6.16 | 1.40176249 | 0.07 | 0.10 | 0.08 | 1.27 | 0.01 |
| | 1437 | | | | | | | | | |
| Enamine2 | T5242217 | M | C | 4.79 | 0.00498275 | 0.06 | 0.07 | 0.06 | 2.58 | 0.55 |
| Enamine2 | T0513- | W | C | 3.45 | 0.23308613 | 0.04 | 0.03 | 0.04 | −1.86 | 0.25 |
| | 0218 | | | | | | | | | |
| Enamine2 | T5211003 | M | C | 4.50 | 0.4804257 | 0.05 | 0.05 | 0.05 | −0.66 | 0.09 |
| Enamine2 | T0520- | M | C | 4.18 | 1.06042176 | 0.04 | 0.05 | 0.04 | −0.03 | 0.54 |
| | 0462 | | | | | | | | | |
| Enamine2 | T5211106 | W | C | 3.58 | 0.56567515 | 0.04 | 0.04 | 0.04 | −1.87 | 0.65 |
| Enamine2 | T0513- | M | C | 5.27 | 2.1593855 | 0.04 | 0.06 | 0.05 | −1.38 | 0.08 |
| | 0122 | | | | | | | | | |
| Enamine2 | T0512- | M | C | 4.05 | 0.57380115 | 0.04 | 0.04 | 0.04 | −1.02 | 0.93 |
| | 8635 | | | | | | | | | |
| Enamine2 | T0512- | M | C | 4.15 | 1.08660531 | 0.04 | 0.05 | 0.04 | −1.99 | 0.55 |
| | 9319 | | | | | | | | | |
| Enamine2 | T0513- | S | C | 11.30 | 3.63051472 | 0.10 | 0.13 | 0.11 | −1.25 | 1.13 |
| | 0201 | | | | | | | | | |
| Enamine2 | T5213128 | M | C | 4.55 | 1.57385802 | 0.04 | 0.05 | 0.05 | −1.71 | 1.31 |
| Enamine2 | T5213954 | W | C | 3.16 | 0.19934562 | 0.04 | 0.03 | 0.03 | −1.75 | 0.70 |
| Enamine2 | T5446230 | M | C | 4.06 | 1.21534994 | 0.05 | 0.04 | 0.04 | −0.20 | 0.66 |
| Enamine2 | T5254047* | M | | 5.09 | 0.50688272 | 0.07 | 0.08 | 0.08 | −0.67 | 0.45 |
| Enamine2 | T0506- | M | | 4.48 | 0.79949544 | 0.07 | 0.07 | 0.07 | 0.99 | 0.45 |
| | 5702* | | | | | | | | | |
| Enamine2 | T0514- | S | C | 5.62 | 0.46695405 | 0.07 | 0.07 | 0.07 | −0.05 | 0.21 |
| | 1693 | | | | | | | | | |
| Enamine2 | T0510- | S | C | 28.56 | 3.94039922 | 0.39 | 0.42 | 0.41 | −1.17 | 0.40 |
| | 3476 | | | | | | | | | |
| Enamine2 | T0508- | M | C | 4.04 | 0.47925314 | 0.07 | 0.05 | 0.06 | −1.07 | 1.44 |
| | 0529 | | | | | | | | | |
| Enamine2 | T0506- | S | C | 6.70 | 0.52302417 | 0.11 | 0.11 | 0.11 | 0.14 | 0.78 |
| | 9182 | | | | | | | | | |
| Enamine2 | T0504- | W | C | 3.52 | 0.5942107 | 0.05 | 0.05 | 0.05 | 0.92 | 0.26 |
| | 2965 | | | | | | | | | |
| Enamine2 | T0504- | W | C | 3.61 | 0.12453328 | 0.05 | 0.05 | 0.05 | −0.07 | 1.24 |
| | 3650 | | | | | | | | | |
| Enamine2 | T0504- | M | C | 3.94 | 0.1128373 | 0.06 | 0.05 | 0.06 | −0.60 | 0.05 |
| | 4157 | | | | | | | | | |
| Enamine2 | T0515- | W | C | 3.73 | 0.2467768 | 0.05 | 0.05 | 0.05 | −1.00 | 0.12 |
| | 1876 | | | | | | | | | |
| Enamine2 | T0516- | W | C | 3.42 | 0.38758547 | 0.05 | 0.05 | 0.05 | −0.84 | 0.96 |
| | 2435 | | | | | | | | | |
| Enamine2 | T0516- | W | C | 3.52 | 0.23056777 | 0.06 | 0.04 | 0.05 | 0.61 | 0.50 |
| | 3323 | | | | | | | | | |
| Enamine2 | T0516- | M | C | 3.68 | 0.57793223 | 0.05 | 0.05 | 0.05 | 0.46 | 0.64 |
| | 2669 | | | | | | | | | |
| Enamine2 | T0515- | W | C | 3.23 | 0.27649133 | 0.05 | 0.05 | 0.05 | −0.55 | 1.03 |
| | 7211 | | | | | | | | | |
| Enamine2 | T0515- | W | C | 3.15 | 0.19886323 | 0.05 | 0.04 | 0.04 | 0.06 | 0.57 |
| | 7259 | | | | | | | | | |
| Enamine2 | T5340353 | M | C | 4.98 | 1.93385213 | 0.07 | 0.05 | 0.06 | −1.55 | 0.34 |
| Enamine2 | T5539099 | W | C | 3.20 | 0.18146189 | 0.04 | 0.05 | 0.05 | 0.96 | 1.55 |
| Enamine2 | T5539103 | W | C | 3.37 | 0.10982452 | 0.04 | 0.05 | 0.05 | 1.84 | 0.32 |
| Enamine2 | T5535170 | M | C | 4.37 | 0.73150018 | 0.28 | 0.33 | 0.31 | −0.10 | 0.34 |
| Enamine2 | T5280552 | M | C | 4.33 | 0.41216216 | 0.28 | 0.27 | 0.27 | −0.32 | 0.41 |
| Enamine2 | T5307314* | S | | 6.30 | 0.17051327 | 0.42 | 0.37 | 0.40 | 0.84 | 0.17 |
| Enamine2 | T5342342 | M | C | 4.52 | 0.28470754 | 0.30 | 0.27 | 0.28 | 0.36 | 0.16 |
| Enamine2 | T0507- | W | C | 3.30 | 0.05645919 | 0.22 | 0.21 | 0.21 | 0.10 | 0.31 |
| | 8998 | | | | | | | | | |
| Enamine2 | T0515- | S | | 10.34 | 1.16435166 | 0.70 | 0.67 | 0.68 | 0.20 | 0.37 |
| | 1927* | | | | | | | | | |
| Enamine2 | T5365031 | M | C | 4.17 | 0.19684783 | 0.27 | 0.28 | 0.28 | 0.74 | 1.57 |
| Enamine2 | T5371551 | S | C | 9.04 | 1.1499587 | 0.61 | 0.58 | 0.60 | −0.57 | 2.22 |
| Enamine2 | T5298850 | M | C | 3.96 | 0.18671189 | 0.26 | 0.27 | 0.26 | −0.10 | 1.96 |
| Enamine2 | T0502- | M | | 4.92 | 0.01422063 | 0.32 | 0.30 | 0.31 | 1.75 | 1.25 |
| | 3042* | | | | | | | | | |
| Enamine2 | T0505- | M | C | 4.29 | 0.11376193 | 0.29 | 0.26 | 0.27 | 0.86 | 0.79 |
| | 5362 | | | | | | | | | |
| Enamine2 | T0507- | W | C | 3.47 | 0.21748836 | 0.22 | 0.22 | 0.22 | 0.37 | 0.03 |
| | 1528 | | | | | | | | | |
| Enamine2 | T5429877 | S | C | 6.24 | 0.92539825 | 0.42 | 0.44 | 0.43 | 0.98 | 0.91 |
| Enamine2 | T0515- | M | | 4.00 | 1.01575486 | 0.26 | 0.23 | 0.25 | 0.98 | 1.56 |
| | 9025* | | | | | | | | | |
| Enamine2 | T0509- | S | C | 7.16 | 0.9828582 | 0.44 | 0.46 | 0.45 | −1.71 | 0.25 |
| | 8494 | | | | | | | | | |
| Enamine2 | T0514- | M | | 4.79 | 1.35867474 | 0.32 | 0.27 | 0.30 | 0.03 | 0.16 |
| | 3358* | | | | | | | | | |
| Enamine2 | T5342902 | W | C | 3.41 | 0.39560673 | 0.21 | 0.22 | 0.22 | −0.43 | 0.38 |
| Enamine2 | T0513- | M | | 5.36 | 1.60786355 | 0.38 | 0.29 | 0.33 | −0.60 | 1.59 |
| | 7724* | | | | | | | | | |
| Enamine2 | T5243726 | M | C | 5.07 | 1.3817578 | 0.25 | 0.33 | 0.29 | −0.60 | 1.92 |
| Selleck | S2269* | M | | 4.15 | 1.520024 | 0.21 | 0.17 | 0.19 | 0.75 | 0.24 |
| Selleck | S2058 | W | C | 3.75 | 0.27041988 | 0.16 | 0.19 | 0.18 | 0.35 | 0.45 |
| Selleck | S2127 | M | C | 3.63 | 0.7144869 | 0.17 | 0.17 | 0.17 | −0.70 | 1.24 |
| Selleck | S2621 | M | C | 6.88 | 2.93805797 | 0.43 | 0.26 | 0.35 | 0.69 | 1.99 |
| Selleck | S2743 | S | C | 8.74 | 0.96301839 | 0.45 | 0.44 | 0.45 | 0.00 | 1.37 |
| Selleck | S2453* | M | | 5.09 | 1.02694498 | 0.21 | 0.32 | 0.26 | 1.00 | 0.62 |
| Selleck | S2670 | W | C | 3.67 | 0.2383094 | 0.17 | 0.21 | 0.19 | 0.07 | 0.89 |
| Selleck | S2686 | S | C | 7.03 | 0.8090961 | 0.37 | 0.35 | 0.36 | 1.33 | 1.05 |
| Selleck | S2749* | S | C | 6.76 | 0.85208774 | 0.35 | 0.34 | 0.34 | −0.10 | 0.93 |
| Selleck | S2401* | M | | 4.93 | 0.43782101 | 0.22 | 0.29 | 0.25 | 0.36 | 1.35 |

* = PAINS flagged

TABLE 2B
SpCas9 eGFP disruption

| | | | | Z score | Z socre | Normalized | Normalized |
|---|---|---|---|---|---|---|---|
| Type | Ctrl type | Rep1 | Rep2 | Rep1 | Rep2 | Z rep1 | Z rep2 |
| Cas9 | Lower | 56.386 | 56.403 | −0.57 | 0.59 | −0.04 | 0.03 |
| Cas9 | Lower | 61.838 | 55.764 | 1.55 | 0.25 | 0.11 | 0.01 |
| Cas9 | Lower | 56.302 | 56.544 | −0.60 | 0.67 | −0.04 | 0.03 |
| Cas9 | Lower | 54.945 | 56.359 | −1.13 | 0.57 | −0.08 | 0.03 |
| Cas9 | Lower | 56.302 | 57.333 | −0.60 | 1.09 | −0.04 | 0.05 |
| Cas9 | Lower | 57.306 | 56.849 | −0.21 | 0.83 | −0.01 | 0.04 |
| Cas9 | Lower | 59.093 | 54.391 | 0.49 | −0.49 | 0.03 | −0.02 |
| Cas9 | Lower | 62.574 | 54.994 | 1.84 | −0.17 | 0.13 | −0.01 |
| Cas9 | Lower | 57.894 | 52.624 | 0.02 | −1.45 | 0.00 | −0.07 |
| Cas9 | Lower | 55.803 | 51.807 | −0.79 | −1.89 | −0.06 | −0.09 |
| Cas9 | Lower | 51.387 | 66.751 | −0.97 | 0.82 | −0.16 | 0.17 |
| Cas9 | Lower | 49.728 | 63.249 | −1.27 | 0.19 | −0.21 | 0.04 |
| Cas9 | Lower | 49.529 | 55.125 | −1.31 | −1.25 | −0.22 | −0.25 |
| Cas9 | Lower | 53.178 | 67.641 | −0.66 | 0.97 | −0.11 | 0.20 |
| Cas9 | Lower | 59.987 | 50.145 | 0.56 | −2.13 | 0.09 | −0.43 |
| Cas9 | Lower | 62.575 | 62.305 | 1.02 | 0.03 | 0.17 | 0.01 |
| Cas9 | Lower | 63.699 | 65.496 | 1.22 | 0.59 | 0.20 | 0.12 |
| Cas9 | Lower | 63.652 | 66.468 | 1.21 | 0.77 | 0.20 | 0.15 |
| Cas9 | Lower | 57.448 | 64.346 | 0.10 | 0.39 | 0.02 | 0.08 |
| Cas9 | Lower | 57.448 | 60.028 | 0.10 | −0.38 | 0.02 | −0.08 |
| Cas9 | Lower | 60.977 | 67.861 | −1.83 | 1.58 | −0.25 | 0.17 |
| Cas9 | Lower | 65.739 | 63.23 | −0.39 | 0.05 | −0.05 | 0.01 |
| Cas9 | Lower | 63.192 | 64.304 | −1.16 | 0.40 | −0.16 | 0.04 |
| Cas9 | Lower | 66.322 | 60.914 | −0.21 | −0.72 | −0.03 | −0.08 |
| Cas9 | Lower | 71.494 | 63.3 | 1.36 | 0.07 | 0.19 | 0.01 |
| Cas9 | Lower | 67.6 | 57.559 | 0.18 | −1.83 | 0.02 | −0.20 |
| Cas9 | Lower | 71.296 | 60.42 | 1.30 | −0.88 | 0.18 | −0.09 |
| Cas9 | Lower | 65.119 | 59.392 | −0.57 | −1.22 | −0.08 | −0.13 |
| Cas9 | Lower | 65.961 | 63.641 | −0.32 | 0.18 | −0.04 | 0.02 |
| Cas9 | Lower | 69.475 | 66.692 | 0.75 | 1.20 | 0.10 | 0.13 |
| Cas9 | Lower | 65.933 | 64.608 | −0.33 | 0.51 | −0.05 | 0.05 |
| Cas9 | Lower | 71.041 | 65.096 | 1.22 | 0.67 | 0.17 | 0.07 |
| Cas9 | Lower | 70.24 | 65.191 | 1.95 | −0.65 | 0.21 | −0.04 |
| Cas9 | Lower | 66.256 | 65.906 | 0.54 | −0.21 | 0.06 | −0.01 |
| Cas9 | Lower | 67.86 | 68.22 | 1.11 | 1.19 | 0.12 | 0.08 |
| Cas9 | Lower | 66.52 | 64.071 | 0.64 | −1.33 | 0.07 | −0.09 |
| Cas9 | Lower | 62.253 | 65.866 | −0.87 | −0.24 | −0.09 | −0.02 |
| Cas9 | Lower | 64.469 | 68.876 | −0.09 | 1.59 | −0.01 | 0.11 |
| Cas9 | Lower | 63.873 | 64.427 | −0.30 | −1.11 | −0.03 | −0.07 |
| Cas9 | Lower | 61.327 | 65.502 | −1.19 | −0.46 | −0.13 | −0.03 |
| Cas9 | Lower | 65.038 | 65.069 | 0.12 | −0.72 | 0.01 | −0.05 |
| Cas9 | Lower | 65.2 | 65.71 | 0.17 | −0.33 | 0.02 | −0.02 |
| Cas9 | Lower | 63.491 | 67.829 | −0.43 | 0.95 | −0.05 | 0.06 |
| Cas9 | Lower | 60.006 | 68.45 | −1.66 | 1.33 | −0.18 | 0.09 |
| Cas9 | Lower | 73.959 | 75.356 | −1.00 | −0.64 | −0.10 | −0.03 |
| Cas9 | Lower | 73.028 | 76.007 | −1.52 | 0.14 | −0.15 | 0.01 |
| Cas9 | Lower | 75.62 | 76.87 | −0.09 | 1.18 | −0.01 | 0.05 |
| Cas9 | Lower | 74.443 | 77.776 | −0.73 | 2.27 | −0.07 | 0.10 |
| Cas9 | Lower | 73.906 | 74.821 | −1.03 | −1.29 | −0.10 | −0.06 |
| Cas9 | Lower | 76.08 | 75.296 | 0.17 | −0.72 | 0.02 | −0.03 |
| Cas9 | Lower | 74.86 | 74.898 | −0.50 | −1.19 | −0.05 | −0.05 |
| Cas9 | Lower | 75.684 | 75.831 | −0.05 | −0.07 | 0.00 | 0.00 |
| Cas9 | Lower | 77.951 | 75.564 | 1.20 | −0.39 | 0.12 | −0.02 |
| Cas9 | Lower | 78.396 | 75.905 | 1.45 | 0.02 | 0.14 | 0.00 |
| Cas9 | Lower | 77.432 | 76.064 | 0.91 | 0.21 | 0.09 | 0.01 |
| Cas9 | Lower | 77.941 | 76.312 | 1.20 | 0.50 | 0.12 | 0.02 |
| Cas9 | Lower | 75 | 76.271 | −0.36 | −0.01 | −0.06 | 0.00 |
| Cas9 | Lower | 78.925 | 76.114 | 0.98 | −0.09 | 0.15 | −0.01 |
| Cas9 | Lower | 72.685 | 75.424 | −1.15 | −0.44 | −0.18 | −0.05 |
| Cas9 | Lower | 77.971 | 74.074 | 0.65 | −1.12 | 0.10 | −0.12 |
| Cas9 | Lower | 79.185 | 74.993 | 1.07 | −0.66 | 0.16 | −0.07 |
| Cas9 | Lower | 68.44 | 75.396 | −2.61 | −0.46 | −0.40 | −0.05 |
| Cas9 | Lower | 77.41 | 75.317 | 0.46 | −0.50 | 0.07 | −0.05 |
| Cas9 | Lower | 77.362 | 73.16 | 0.45 | −1.58 | 0.07 | −0.17 |
| Cas9 | Lower | 75.696 | 76.603 | −0.12 | 0.15 | −0.02 | 0.02 |
| Cas9 | Lower | 75.831 | 75.129 | −0.08 | −0.59 | −0.01 | −0.06 |
| Cas9 | Lower | 73.082 | 74.261 | −1.02 | −1.03 | −0.16 | −0.11 |
| Cas9 | Lower | 76.415 | 77.567 | 0.12 | 0.64 | 0.02 | 0.07 |
| Ctrl | Upper | 94.147 | 95.657 | 14.11 | 21.77 | 0.99 | 1.05 |
| Ctrl | Upper | 95.87 | 94.995 | 14.77 | 21.41 | 1.03 | 1.03 |
| Ctrl | Upper | 94.552 | 91.99 | 14.26 | 19.79 | 1.00 | 0.96 |
| Ctrl | Upper | 95.012 | 93.49 | 14.44 | 20.60 | 1.01 | 0.99 |
| Ctrl | Upper | 94.273 | 92.499 | 14.15 | 20.07 | 0.99 | 0.97 |
| Ctrl | Upper | 95.692 | 93.791 | 14.71 | 20.76 | 1.03 | 1.00 |
| Ctrl | Upper | 92.506 | 94.572 | 13.47 | 21.19 | 0.94 | 1.02 |
| Ctrl | Upper | 95.534 | 93.021 | 14.64 | 20.35 | 1.02 | 0.98 |
| Ctrl | Upper | 95.525 | 94.431 | 14.64 | 21.11 | 1.02 | 1.02 |
| Ctrl | Upper | 93.042 | 92.671 | 13.68 | 20.16 | 0.96 | 0.97 |
| Ctrl | Upper | 89.912 | 84.982 | 5.88 | 4.06 | 0.98 | 0.82 |
| Ctrl | Upper | 86.254 | 85.79 | 5.23 | 4.20 | 0.87 | 0.85 |
| Ctrl | Upper | 93.264 | 88.725 | 6.48 | 4.72 | 1.08 | 0.95 |
| Ctrl | Upper | 87.753 | 91.428 | 5.50 | 5.20 | 0.92 | 1.05 |
| Ctrl | Upper | 90.798 | 92.774 | 6.04 | 5.44 | 1.01 | 1.10 |
| Ctrl | Upper | 88.737 | 89.9 | 5.67 | 4.93 | 0.95 | 1.00 |
| Ctrl | Upper | 88.953 | 91.215 | 5.71 | 5.16 | 0.95 | 1.04 |
| Ctrl | Upper | 93.185 | 88.722 | 6.46 | 4.72 | 1.08 | 0.95 |
| Ctrl | Upper | 93.386 | 94.019 | 6.50 | 5.66 | 1.08 | 1.14 |
| Ctrl | Upper | 93.386 | 92.417 | 6.50 | 5.38 | 1.08 | 1.09 |
| Ctrl | Upper | 93.167 | 92.275 | 7.92 | 9.68 | 1.09 | 1.04 |
| Ctrl | Upper | 93.763 | 91.136 | 8.10 | 9.30 | 1.12 | 1.00 |
| Ctrl | Upper | 90.196 | 93.4 | 7.02 | 10.05 | 0.97 | 1.08 |
| Ctrl | Upper | 92.031 | 92.169 | 7.57 | 9.64 | 1.05 | 1.04 |
| Ctrl | Upper | 88.515 | 89.989 | 6.51 | 8.92 | 0.90 | 0.96 |
| Ctrl | Upper | 91.423 | 89.171 | 7.39 | 8.65 | 1.02 | 0.93 |
| Ctrl | Upper | 90.81 | 89.151 | 7.20 | 8.64 | 1.00 | 0.93 |
| Ctrl | Upper | 90.963 | 90.35 | 7.25 | 9.04 | 1.00 | 0.97 |
| Ctrl | Upper | 89.06 | 89.609 | 6.67 | 8.80 | 0.92 | 0.94 |
| Ctrl | Upper | 90.754 | 92.386 | 7.19 | 9.72 | 0.99 | 1.04 |
| Ctrl | Upper | 91.421 | 92.159 | 7.39 | 9.64 | 1.02 | 1.03 |
| Ctrl | Upper | 88.764 | 92.381 | 6.58 | 9.71 | 0.91 | 1.04 |
| Ctrl | Upper | 90.519 | 85.628 | 9.11 | 11.76 | 0.99 | 0.79 |
| Ctrl | Upper | 89.229 | 87.884 | 8.65 | 13.13 | 0.94 | 0.88 |
| Ctrl | Upper | 93.799 | 90.587 | 10.26 | 14.78 | 1.11 | 0.99 |
| Ctrl | Upper | 92.291 | 91.515 | 9.73 | 15.34 | 1.05 | 1.03 |
| Ctrl | Upper | 88.915 | 84.91 | 8.54 | 11.33 | 0.92 | 0.76 |
| Ctrl | Upper | 92.251 | 92.858 | 9.72 | 16.16 | 1.05 | 1.09 |
| Ctrl | Upper | 91.125 | 93.368 | 9.32 | 16.47 | 1.01 | 1.11 |
| Ctrl | Upper | 92.155 | 89.759 | 9.68 | 14.27 | 1.05 | 0.96 |
| Ctrl | Upper | 87.216 | 93.888 | 7.94 | 16.78 | 0.86 | 1.13 |
| Ctrl | Upper | 87.635 | 91.624 | 8.09 | 15.41 | 0.88 | 1.04 |
| Ctrl | Upper | 92.563 | 93.608 | 9.83 | 16.61 | 1.06 | 1.12 |
| Ctrl | Upper | 93.037 | 93.422 | 9.99 | 16.50 | 1.08 | 1.11 |
| Ctrl | Upper | 93.631 | 96.215 | 9.85 | 24.43 | 0.95 | 1.07 |
| Ctrl | Upper | 93.896 | 94.422 | 10.00 | 22.27 | 0.96 | 0.97 |
| Ctrl | Upper | 95.202 | 94.544 | 10.72 | 22.42 | 1.03 | 0.98 |
| Ctrl | Upper | 93.309 | 94.045 | 9.68 | 21.82 | 0.93 | 0.95 |
| Ctrl | Upper | 94.037 | 95.963 | 10.08 | 24.12 | 0.97 | 1.05 |
| Ctrl | Upper | 95.367 | 94.686 | 10.81 | 22.59 | 1.04 | 0.99 |
| Ctrl | Upper | 94.86 | 95.613 | 10.53 | 23.70 | 1.01 | 1.04 |
| Ctrl | Upper | 94.99 | 95.625 | 10.61 | 23.72 | 1.02 | 1.04 |
| Ctrl | Upper | 94.242 | 96.608 | 10.19 | 24.90 | 0.98 | 1.09 |
| Ctrl | Upper | 95.151 | 95.857 | 10.69 | 24.00 | 1.03 | 1.05 |
| Ctrl | Upper | 95.723 | 93.092 | 11.01 | 20.67 | 1.06 | 0.90 |
| Ctrl | Upper | 94.598 | 92.459 | 10.39 | 19.91 | 1.00 | 0.87 |
| Ctrl | Upper | 94.412 | 92.469 | 6.28 | 8.17 | 0.96 | 0.85 |
| Ctrl | Upper | 95.729 | 94.661 | 6.73 | 9.27 | 1.03 | 0.97 |
| Ctrl | Upper | 92.195 | 96.653 | 5.52 | 10.28 | 0.84 | 1.07 |
| Ctrl | Upper | 95.302 | 93.32 | 6.58 | 8.60 | 1.01 | 0.90 |
| Ctrl | Upper | 95.899 | 96.013 | 6.79 | 9.96 | 1.04 | 1.04 |
| Ctrl | Upper | 95.572 | 95.531 | 6.68 | 9.71 | 1.02 | 1.01 |
| Ctrl | Upper | 95.044 | 95.53 | 6.50 | 9.71 | 0.99 | 1.01 |
| Ctrl | Upper | 95.754 | 95.006 | 6.74 | 9.45 | 1.03 | 0.98 |
| Ctrl | Upper | 94.927 | 96.023 | 6.46 | 9.96 | 0.99 | 1.04 |
| Ctrl | Upper | 96.67 | 96.344 | 7.05 | 10.12 | 1.08 | 1.05 |
| Ctrl | Upper | 95.543 | 95.55 | 6.67 | 9.72 | 1.02 | 1.01 |
| Ctrl | Upper | 94.889 | 96.657 | 6.44 | 10.28 | 0.99 | 1.07 |
| G786-2334 | | 99.55 | 99.676 | 13.12 | 28.59 | 1.26 | 1.25 |
| G786-2325 | | 98.877 | 98.53 | 12.75 | 27.21 | 1.23 | 1.19 |
| T5242217 | | 82.667 | 80.63 | 9.64 | 13.66 | 0.68 | 0.66 |
| G786-1325 | | 86.012 | 86.243 | 5.65 | 12.44 | 0.54 | 0.54 |
| G786-1572 | | 82.405 | 84.887 | 3.66 | 10.81 | 0.35 | 0.47 |
| G786-1547 | | 82.802 | 84.086 | 3.88 | 9.85 | 0.37 | 0.43 |
| G786-1264 | | 82.717 | 83.951 | 3.83 | 9.69 | 0.37 | 0.42 |
| T5461482 | | 66.842 | 67.113 | 3.50 | 6.37 | 0.24 | 0.31 |
| T0503-6911 | | 67.522 | 66.588 | 3.76 | 6.09 | 0.26 | 0.29 |
| T5535170 | | 67.964 | 65.339 | 3.93 | 5.41 | 0.28 | 0.26 |
| G786-1324 | | 81.427 | 79.939 | 3.12 | 4.86 | 0.30 | 0.21 |
| T5371551 | | 64.278 | 76.754 | 2.50 | 11.57 | 0.17 | 0.56 |
| T5264279 | | 65.176 | 67.722 | 2.85 | 6.70 | 0.20 | 0.32 |
| G946-0488 | | 79.392 | 79.926 | 2.00 | 4.85 | 0.19 | 0.21 |
| T5213954 | | 64.255 | 63.424 | 2.49 | 4.38 | 0.17 | 0.21 |
| T0504-2965 | | 63.86 | 62.371 | 2.34 | 3.81 | 0.16 | 0.18 |
| T5385382 | | 62.263 | 64.4 | 1.72 | 4.91 | 0.12 | 0.24 |
| G786-1665 | | 77.543 | 79.946 | 0.98 | 4.87 | 0.09 | 0.21 |
| G769-1036 | | 78.973 | 79.726 | 1.77 | 4.61 | 0.17 | 0.20 |
| G786-1263 | | 76.854 | 79.397 | 0.60 | 4.21 | 0.06 | 0.18 |
| T0503-2749 | | 60.821 | 62.831 | 1.16 | 4.06 | 0.08 | 0.20 |
| T5539099 | | 58.64 | 61.814 | 0.31 | 3.51 | 0.02 | 0.17 |
| T5272320 | | 62.825 | 61.016 | 1.94 | 3.08 | 0.14 | 0.15 |
| T0504-1437 | | 68.038 | 56.265 | 3.96 | 0.52 | 0.28 | 0.02 |
| T5451097 | | 65.966 | 42.703 | 3.16 | −6.80 | 0.22 | −0.33 |
| G786-1670 | | 74.875 | 78.651 | −0.50 | 3.32 | −0.05 | 0.14 |
| T5380152 | | 54.565 | 62.745 | −1.27 | 4.01 | −0.09 | 0.19 |
| T5281519 | | 53.71 | 61.429 | −1.61 | 3.30 | −0.11 | 0.16 |
| G786-1602 | | 72.665 | 78.925 | −1.72 | 3.65 | −0.17 | 0.16 |
| 4130-5308 | | 50.927 | 64.253 | −2.69 | 4.83 | −0.19 | 0.23 |
| T5280552 | | 62.528 | 60.569 | 1.82 | 2.84 | 0.13 | 0.14 |
| G786-1570 | | 79.315 | 78.182 | 1.95 | 2.75 | 0.19 | 0.12 |
| T0519-6400 | | 62.891 | 59.8 | 1.96 | 2.42 | 0.14 | 0.12 |
| T0507-1528 | | 61.382 | 59.247 | 1.37 | 2.13 | 0.10 | 0.10 |
| L662-0987 | | 79.157 | 79.829 | 1.06 | 1.78 | 0.16 | 0.19 |
| G771-0374 | | 79.068 | 77.189 | 1.82 | 1.56 | 0.18 | 0.07 |
| T0503-9777 | | 60.694 | 57.686 | 1.11 | 1.28 | 0.08 | 0.06 |
| G786-1534 | | 79.44 | 76.727 | 2.02 | 1.00 | 0.19 | 0.04 |
| G786-1471 | | 78.179 | 76.202 | 1.33 | 0.37 | 0.13 | 0.02 |
| cpd176 | | 63.034 | 60.179 | 1.10 | −0.35 | 0.18 | −0.07 |
| D727-0165 | | 70.62 | 61.783 | 1.09 | −0.43 | 0.15 | −0.05 |
| cpd99 | | 64.556 | 58.903 | 1.37 | −0.58 | 0.23 | −0.12 |
| cpd144 | | 66.18 | 58.554 | 1.66 | −0.64 | 0.28 | −0.13 |
| cpd153 | | 67.763 | 58.097 | 1.94 | −0.72 | 0.32 | −0.15 |
| cpd98 | | 67.658 | 57.76 | 1.92 | −0.78 | 0.32 | −0.16 |
| cpd145 | | 69.038 | 57.651 | 2.17 | −0.80 | 0.36 | −0.16 |
| G786-2194 | | 78.34 | 75.14 | 1.42 | −0.90 | 0.14 | −0.04 |
| cpd185 | | 62.696 | 56.912 | 1.04 | −0.93 | 0.17 | −0.19 |
| cpd137 | | 62.997 | 55.358 | 1.09 | −1.21 | 0.18 | −0.24 |
| cpd161 | | 63.041 | 54.146 | 1.10 | −1.42 | 0.18 | −0.29 |
| cpd138 | | 66.234 | 48.526 | 1.67 | −2.42 | 0.28 | −0.49 |
| Cpd84 | | 60.849 | 49.755 | 1.17 | −3.00 | 0.08 | −0.14 |
| G786-1551 | | 77.371 | 76.453 | 0.88 | 0.67 | 0.08 | 0.03 |
| Cpd63 | | 60.105 | 52.593 | 0.88 | −1.46 | 0.06 | −0.07 |
| cpd157 | | 61.746 | 54.302 | 0.87 | −1.40 | 0.14 | −0.28 |
| cpd104 | | 61.705 | 51.504 | 0.86 | −1.89 | 0.14 | −0.38 |
| cpd122 | | 61.68 | 52.69 | 0.86 | −1.68 | 0.14 | −0.34 |
| L662-0973 | | 78.528 | 78.814 | 0.85 | 1.27 | 0.13 | 0.13 |
| cpd154 | | 61.346 | 45.479 | 0.80 | −2.96 | 0.13 | −0.60 |
| Cpd64 | | 59.842 | 57.874 | 0.78 | 1.39 | 0.05 | 0.07 |
| cpd118 | | 61.222 | 55.91 | 0.78 | −1.11 | 0.13 | −0.22 |
| L063-0010 | | 77.098 | 74.649 | 0.73 | −1.49 | 0.07 | −0.07 |
| cpd164 | | 60.922 | 51.698 | 0.72 | −1.86 | 0.12 | −0.38 |
| J021-0103 | | 76.98 | 76.146 | 0.66 | 0.31 | 0.06 | 0.01 |
| F170-0052 | | 69.064 | 58.318 | 0.62 | −1.58 | 0.09 | −0.17 |
| cpd178 | | 60.262 | 57.9 | 0.60 | −0.76 | 0.10 | −0.15 |
| L663-0999 | | 77.659 | 74.256 | 0.55 | −1.03 | 0.08 | −0.11 |
| cpd168 | | 59.758 | 59.726 | 0.52 | −0.43 | 0.09 | −0.09 |
| cpd167 | | 59.734 | 52.865 | 0.51 | −1.65 | 0.09 | −0.33 |
| cpd184 | | 59.712 | 57.336 | 0.51 | −0.86 | 0.08 | −0.17 |
| cpd158 | | 59.677 | 61.894 | 0.50 | −0.05 | 0.08 | −0.01 |
| M040-0428 | | 77.507 | 79.147 | 0.50 | 1.44 | 0.08 | 0.15 |
| S2058 | | 77.475 | 77.936 | 0.48 | 0.83 | 0.07 | 0.09 |
| D727-0786 | | 68.596 | 68.839 | 0.48 | 1.91 | 0.07 | 0.20 |
| cpd102 | | 59.522 | 56.238 | 0.47 | −1.05 | 0.08 | −0.21 |
| cpd174 | | 59.492 | 57.55 | 0.47 | −0.82 | 0.08 | −0.17 |
| L538-0006 | | 77.401 | 72.973 | 0.46 | −1.68 | 0.07 | −0.17 |
| M040-0118 | | 77.349 | 75.653 | 0.44 | −0.33 | 0.07 | −0.03 |
| Cpd44 | | 58.95 | 55.952 | 0.43 | 0.35 | 0.03 | 0.02 |
| Cpd42 | | 58.884 | 53.493 | 0.40 | −0.98 | 0.03 | −0.05 |
| G748-0016 | | 65.797 | 58.369 | 0.38 | −4.79 | 0.04 | −0.32 |
| L662-0604 | | 77.166 | 76.48 | 0.38 | 0.09 | 0.06 | 0.01 |
| D727-0713 | | 68.26 | 66.347 | 0.38 | 1.08 | 0.05 | 0.12 |
| L662-0686 | | 77.106 | 73.767 | 0.36 | −1.28 | 0.05 | −0.13 |
| cpd175 | | 58.636 | 56.719 | 0.32 | −0.97 | 0.05 | −0.20 |
| D727-0351 | | 67.967 | 67.06 | 0.29 | 1.32 | 0.04 | 0.14 |
| cpd165 | | 58.474 | 61.062 | 0.29 | −0.19 | 0.05 | −0.04 |
| cpd143 | | 58.415 | 48.82 | 0.28 | −2.37 | 0.05 | −0.48 |
| G946-0601 | | 76.265 | 74.316 | 0.27 | −1.89 | 0.03 | −0.08 |
| G771-0699 | | 76.238 | 77.111 | 0.26 | 1.47 | 0.02 | 0.06 |
| cpd107 | | 58.26 | 58.574 | 0.25 | −0.64 | 0.04 | −0.13 |
| F083-0022 | | 67.708 | 62.217 | 0.21 | −0.29 | 0.03 | −0.03 |
| Cpd21 | | 58.382 | 52.214 | 0.21 | −1.67 | 0.01 | −0.08 |
| cpd129 | | 57.965 | 61.673 | 0.20 | −0.09 | 0.03 | −0.02 |
| cpd127 | | 57.953 | 62.438 | 0.19 | 0.05 | 0.03 | 0.01 |
| cpd186 | | 57.934 | 52.751 | 0.19 | −1.67 | 0.03 | −0.34 |
| G786-0269 | | 76.118 | 76.574 | 0.19 | 0.82 | 0.02 | 0.04 |
| Cpd18 | | 58.308 | 55.133 | 0.18 | −0.09 | 0.01 | 0.00 |
| SAM001246816 | | 76.555 | 74.015 | 0.17 | −1.15 | 0.03 | −0.12 |
| cpd109 | | 57.8 | 57.523 | 0.17 | −0.82 | 0.03 | −0.17 |
| Cpd33 | | 58.236 | 55.57 | 0.15 | 0.14 | 0.01 | 0.01 |
| Cpd74 | | 58.21 | 51.999 | 0.14 | −1.78 | 0.01 | −0.09 |
| G786-1669 | | 76.013 | 74.373 | 0.13 | −1.83 | 0.01 | −0.08 |
| D727-0051 | | 67.429 | 65.459 | 0.13 | 0.79 | 0.02 | 0.08 |
| cpd106 | | 57.499 | 58.155 | 0.11 | −0.71 | 0.02 | −0.14 |
| Cpd10 | | 58.004 | 60.748 | 0.06 | 2.94 | 0.00 | 0.14 |
| cpd181 | | 57.193 | 48.111 | 0.06 | −2.49 | 0.01 | −0.50 |
| cpd135 | | 57.027 | 49.155 | 0.03 | −2.31 | 0.00 | −0.47 |
| cpd111 | | 56.98 | 62.004 | 0.02 | −0.03 | 0.00 | −0.01 |
| L921-1051 | | 76.114 | 74.779 | 0.02 | −0.77 | 0.00 | −0.08 |
| L662-0597 | | 76.108 | 73.535 | 0.02 | −1.39 | 0.00 | −0.15 |
| cpd124 | | 56.918 | 51.511 | 0.01 | −1.89 | 0.00 | −0.38 |
| M060-0419 | | 76.082 | 76.939 | 0.01 | 0.32 | 0.00 | 0.03 |
| L921-0997 | | 76.081 | 77.332 | 0.01 | 0.52 | 0.00 | 0.05 |
| L662-0693 | | 76.04 | 78.064 | −0.01 | 0.89 | 0.00 | 0.09 |
| cpd97 | | 56.801 | 51.48 | −0.01 | −1.90 | 0.00 | −0.38 |
| cpd163 | | 56.738 | 69.307 | −0.02 | 1.27 | 0.00 | 0.26 |
| M184-0111 | | 75.953 | 71.294 | −0.04 | −2.53 | −0.01 | −0.26 |
| G774-0231 | | 75.709 | 77.501 | −0.04 | 1.93 | 0.00 | 0.08 |
| cpd146 | | 56.435 | 61.684 | −0.08 | −0.08 | −0.01 | −0.02 |
| Cpd40 | | 57.638 | 51.826 | −0.08 | −1.88 | −0.01 | −0.09 |
| G786-1537 | | 75.606 | 76.173 | −0.09 | 0.34 | −0.01 | 0.01 |
| cpd155 | | 56.31 | 60.912 | −0.10 | −0.22 | −0.02 | −0.04 |
| M040-0079 | | 75.764 | 74.38 | −0.10 | −0.97 | −0.02 | −0.10 |
| cpd160 | | 56.293 | 54.014 | −0.10 | −1.45 | −0.02 | −0.29 |
| cpd131 | | 56.197 | 55.431 | −0.12 | −1.19 | −0.02 | −0.24 |
| G786-0317 | | 75.549 | 76.545 | −0.12 | 0.79 | −0.01 | 0.03 |
| cpd103 | | 56.121 | 52.923 | −0.13 | −1.64 | −0.02 | −0.33 |
| cpd188 | | 56.096 | 56.096 | −0.14 | −1.08 | −0.02 | −0.22 |
| cpd147 | | 56.069 | 47.145 | −0.14 | −2.67 | −0.02 | −0.54 |
| cpd166 | | 56.023 | 51.435 | −0.15 | −1.90 | −0.02 | −0.39 |
| Cpd48 | | 57.452 | 53.108 | −0.15 | −1.19 | −0.01 | −0.06 |
| G771-0015 | | 75.479 | 74.168 | −0.16 | −2.07 | −0.02 | −0.09 |
| cpd123 | | 55.919 | 58.313 | −0.17 | −0.68 | −0.03 | −0.14 |
| L662-0977 | | 75.519 | 74.871 | −0.18 | −0.72 | −0.03 | −0.08 |
| J024-0623 | | 75.404 | 74.649 | −0.20 | −1.49 | −0.02 | −0.07 |
| L663-0996 | | 75.44 | 69.294 | −0.21 | −3.54 | −0.03 | −0.37 |
| M040-0084 | | 75.437 | 73.212 | −0.21 | −1.56 | −0.03 | −0.16 |
| D727-0743 | | 66.254 | 59.503 | −0.23 | −1.19 | −0.03 | −0.13 |
| cpd100 | | 55.423 | 51.001 | −0.26 | −1.98 | −0.04 | −0.40 |
| cpd180 | | 55.387 | 52.595 | −0.26 | −1.70 | −0.04 | −0.34 |
| F523-0549 | | 63.953 | 60.854 | −0.27 | −3.28 | −0.03 | −0.22 |
| cpd169 | | 55.294 | 43.453 | −0.28 | −3.32 | −0.05 | −0.67 |
| L662-0655 | | 75.165 | 77.177 | −0.31 | 0.44 | −0.05 | 0.05 |
| cpd172 | | 55.139 | 53.246 | −0.31 | −1.58 | −0.05 | −0.32 |
| F086-0032 | | 65.998 | 60.471 | −0.31 | −0.87 | −0.04 | −0.09 |
| cpd113 | | 55.089 | 57.781 | −0.32 | −0.78 | −0.05 | −0.16 |
| Cpd66 | | 57.029 | 51.962 | −0.32 | −1.80 | −0.02 | −0.09 |
| F128-0041 | | 65.885 | 58.95 | −0.34 | −1.37 | −0.05 | −0.15 |
| L662-0678 | | 74.99 | 76.074 | −0.37 | −0.11 | −0.06 | −0.01 |
| G786-0272 | | 75.094 | 76.951 | −0.38 | 1.27 | −0.04 | 0.06 |
| J006-0457 | | 75.087 | 76.215 | −0.38 | 0.39 | −0.04 | 0.02 |
| cpd149 | | 54.687 | 57.301 | −0.39 | −0.86 | −0.06 | −0.17 |
| Cpd58 | | 56.843 | 52.726 | −0.39 | −1.39 | −0.03 | −0.07 |
| Cpd86 | | 56.824 | 55.099 | −0.40 | −0.11 | −0.03 | −0.01 |
| D727-0394 | | 65.675 | 59.343 | −0.40 | −1.24 | −0.06 | −0.13 |
| cpd128 | | 54.569 | 43.3 | −0.41 | −3.35 | −0.07 | −0.68 |
| cpd117 | | 54.506 | 54.359 | −0.42 | −1.38 | −0.07 | −0.28 |
| F083-0009 | | 65.521 | 54.311 | −0.45 | −2.91 | −0.06 | −0.31 |
| G650-0193 | | 63.431 | 55.676 | −0.45 | −6.43 | −0.05 | −0.43 |
| J024-0800 | | 74.947 | 73.851 | −0.46 | −2.45 | −0.04 | −0.11 |
| cpd119 | | 54.288 | 65.232 | −0.46 | 0.55 | −0.08 | 0.11 |
| cpd140 | | 54.138 | 48.309 | −0.48 | −2.46 | −0.08 | −0.50 |
| M040-0170 | | 74.64 | 74.389 | −0.49 | −0.96 | −0.07 | −0.10 |
| P616-0072 | | 74.607 | 76.04 | −0.50 | −0.13 | −0.08 | −0.01 |
| cpd148 | | 54.063 | 57.64 | −0.50 | −0.80 | −0.08 | −0.16 |
| L921-1004 | | 74.581 | 75.062 | −0.51 | −0.62 | −0.08 | −0.07 |
| E760-4921 | | 65.308 | 56.118 | −0.52 | −2.31 | −0.07 | −0.25 |
| J026-3874 | | 74.839 | 72.206 | −0.52 | −4.43 | −0.05 | −0.19 |
| M040-0116 | | 74.539 | 73.409 | −0.52 | −1.46 | −0.08 | −0.15 |
| G771-0008 | | 74.833 | 76.427 | −0.52 | 0.64 | −0.05 | 0.03 |
| cpd121 | | 53.917 | 48.881 | −0.52 | −2.36 | −0.09 | −0.48 |
| L663-1064 | | 74.434 | 70.767 | −0.56 | −2.79 | −0.08 | −0.29 |
| G786-0400 | | 74.767 | 77.834 | −0.56 | 2.33 | −0.05 | 0.10 |
| J026-0217 | | 74.762 | 75.721 | −0.56 | −0.20 | −0.05 | −0.01 |
| G786-0273 | | 74.755 | 72.066 | −0.56 | −4.60 | −0.05 | −0.20 |
| F128-0076 | | 65.12 | 59.748 | −0.57 | −1.11 | −0.08 | −0.12 |
| cpd189 | | 53.617 | 53.617 | −0.58 | −1.52 | −0.10 | −0.31 |
| cpd105 | | 53.606 | 56.703 | −0.58 | −0.97 | −0.10 | −0.20 |
| G786-2003 | | 74.712 | 75.558 | −0.59 | −0.40 | −0.06 | −0.02 |
| cpd171 | | 53.512 | 50.307 | −0.60 | −2.10 | −0.10 | −0.43 |
| cpd173 | | 53.465 | 62.535 | −0.60 | 0.07 | −0.10 | 0.01 |
| D727-0526 | | 65.01 | 57.026 | −0.61 | −2.01 | −0.08 | −0.22 |
| G786-1588 | | 74.668 | 74.973 | −0.61 | −1.10 | −0.06 | −0.05 |
| D727-0518 | | 64.983 | 61.706 | −0.61 | −0.46 | −0.08 | −0.05 |
| cpd141 | | 53.401 | 47.659 | −0.62 | −2.58 | −0.10 | −0.52 |
| cpd110 | | 53.39 | 55.669 | −0.62 | −1.15 | −0.10 | −0.23 |
| G786-1604 | | 62.953 | 59.339 | −0.62 | −4.20 | −0.07 | −0.28 |
| D727-0523 | | 64.95 | 58.897 | −0.62 | −1.39 | −0.09 | −0.15 |
| Cpd27 | | 56.213 | 59.661 | −0.63 | 2.35 | −0.04 | 0.11 |
| L663-1062 | | 74.182 | 74.913 | −0.64 | −0.70 | −0.10 | −0.07 |
| F083-0067 | | 64.842 | 56.626 | −0.66 | −2.14 | −0.09 | −0.23 |
| G786-1822 | | 74.581 | 75.855 | −0.66 | −0.04 | −0.06 | 0.00 |
| M130-0012 | | 74.104 | 75.755 | −0.67 | −0.27 | −0.10 | −0.03 |
| M040-0082 | | 74.08 | 74.486 | −0.68 | −0.91 | −0.10 | −0.10 |
| L663-1076 | | 74.045 | 76.06 | −0.69 | −0.12 | −0.11 | −0.01 |
| cpd133 | | 52.986 | 60.517 | −0.69 | −0.29 | −0.12 | −0.06 |
| cpd139 | | 52.985 | 62.361 | −0.69 | 0.04 | −0.12 | 0.01 |
| Cpd11 | | 56.047 | 55.984 | −0.70 | 0.37 | −0.05 | 0.02 |
| cpd152 | | 52.89 | 50.912 | −0.71 | −2.00 | −0.12 | −0.40 |
| cpd150 | | 52.884 | 52.15 | −0.71 | −1.78 | −0.12 | −0.36 |
| D727-0490 | | 64.638 | 59.05 | −0.72 | −1.34 | −0.10 | −0.14 |
| L662-0345 | | 73.935 | 70.083 | −0.73 | −3.14 | −0.11 | −0.33 |
| Cpd70 | | 55.966 | 55.621 | −0.73 | 0.17 | −0.05 | 0.01 |
| G786-0423 | | 74.441 | 75.243 | −0.74 | −0.78 | −0.07 | −0.03 |
| G775-0401 | | 74.439 | 77.045 | −0.74 | 1.39 | −0.07 | 0.06 |
| S2127 | | 73.9 | 71.027 | −0.74 | −2.66 | −0.11 | −0.28 |
| cpd115 | | 52.664 | 57.16 | −0.75 | −0.89 | −0.12 | −0.18 |
| cpd187 | | 52.659 | 52.659 | −0.75 | −1.69 | −0.12 | −0.34 |
| L662-0315 | | 73.867 | 75.357 | −0.75 | −0.47 | −0.11 | −0.05 |
| L378-0328 | | 74.416 | 74.832 | −0.75 | −1.27 | −0.07 | −0.06 |
| F483-0122 | | 62.584 | 56.695 | −0.75 | −5.81 | −0.08 | −0.39 |
| cpd134 | | 52.605 | 51.228 | −0.76 | −1.94 | −0.13 | −0.39 |
| J080-0666 | | 74.388 | 72.547 | −0.77 | −4.02 | −0.07 | −0.18 |
| M040-0083 | | 73.819 | 77.355 | −0.77 | 0.53 | −0.12 | 0.06 |
| D727-0066 | | 64.43 | 58.807 | −0.78 | −1.42 | −0.11 | −0.15 |
| F324-0233 | | 62.479 | 67.179 | −0.79 | 0.56 | −0.09 | 0.04 |
| F517-0187 | | 62.479 | 57.518 | −0.79 | −5.31 | −0.09 | −0.36 |
| G786-0335 | | 74.347 | 77.667 | −0.79 | 2.13 | −0.08 | 0.09 |
| L662-0692 | | 73.731 | 76.871 | −0.80 | 0.29 | −0.12 | 0.03 |
| L378-0329 | | 73.724 | 78.669 | −0.80 | 1.20 | −0.12 | 0.12 |
| D727-0121 | | 64.367 | 62.434 | −0.80 | −0.22 | −0.11 | −0.02 |
| F086-0619 | | 64.289 | 57.556 | −0.82 | −1.83 | −0.11 | −0.20 |
| D727-0755 | | 64.271 | 60.694 | −0.83 | −0.79 | −0.11 | −0.09 |
| L662-0654 | | 73.611 | 73.579 | −0.84 | −1.37 | −0.13 | −0.14 |
| cpd156 | | 52.13 | 57.028 | −0.84 | −0.91 | −0.14 | −0.18 |
| M040-0159 | | 73.59 | 74.619 | −0.84 | −0.85 | −0.13 | −0.09 |
| M040-0093 | | 73.586 | 73.743 | −0.85 | −1.29 | −0.13 | −0.13 |
| cpd151 | | 52.097 | 56.678 | −0.85 | −0.97 | −0.14 | −0.20 |
| D727-0522 | | 64.203 | 65.458 | −0.85 | 0.79 | −0.12 | 0.08 |
| M040-0094 | | 73.551 | 74.221 | −0.86 | −1.05 | −0.13 | −0.11 |
| Cpd94 | | 55.635 | 50.955 | −0.86 | −2.35 | −0.06 | −0.11 |
| F518-0049 | | 62.276 | 60.623 | −0.86 | −3.42 | −0.09 | −0.23 |
| D727-0838 | | 64.17 | 57.281 | −0.86 | −1.92 | −0.12 | −0.21 |
| Cpd82 | | 55.604 | 51.108 | −0.87 | −2.27 | −0.06 | −0.11 |
| D727-0753 | | 64.064 | 57.164 | −0.89 | −1.96 | −0.12 | −0.21 |
| F083-0023 | | 64.055 | 59.095 | −0.90 | −1.32 | −0.12 | −0.14 |
| D727-0805 | | 64.053 | 60.131 | −0.90 | −0.98 | −0.12 | −0.11 |
| M115-0551 | | 73.409 | 71.933 | −0.91 | −2.20 | −0.14 | −0.23 |
| F512-0180 | | 62.132 | 61.327 | −0.91 | −3.00 | −0.10 | −0.20 |
| cpd132 | | 51.696 | 58.71 | −0.92 | −0.61 | −0.15 | −0.12 |
| Cpd71 | | 55.466 | 51.857 | −0.92 | −1.86 | −0.06 | −0.09 |
| cpd112 | | 51.611 | 54.911 | −0.93 | −1.29 | −0.16 | −0.26 |
| D727-0883 | | 63.924 | 60.422 | −0.93 | −0.88 | −0.13 | −0.09 |
| L662-0688 | | 73.295 | 74.838 | −0.94 | −0.74 | −0.14 | −0.08 |
| F378-0506 | | 62.032 | 65.398 | −0.95 | −0.52 | −0.10 | −0.04 |
| G769-1010 | | 62.024 | 54.641 | −0.95 | −7.06 | −0.10 | −0.47 |
| Cpd1 | | 55.393 | 54.25 | −0.95 | −0.57 | −0.07 | −0.03 |
| L538-0010 | | 73.269 | 75.198 | −0.95 | −0.56 | −0.15 | −0.06 |
| G786-1559 | | 74.037 | 76.045 | −0.96 | 0.18 | −0.09 | 0.01 |
| Cpd36 | | 55.36 | 53.971 | −0.97 | −0.72 | −0.07 | −0.03 |
| D727-0122 | | 63.824 | 66.933 | −0.97 | 1.28 | −0.13 | 0.14 |
| Cpd87 | | 55.343 | 55.888 | −0.97 | 0.31 | −0.07 | 0.02 |
| P773-6557 | | 73.196 | 72.69 | −0.98 | −1.82 | −0.15 | −0.19 |
| Cpd85 | | 55.319 | 48.96 | −0.98 | −3.42 | −0.07 | −0.17 |
| cpd108 | | 51.345 | 54.273 | −0.98 | −1.40 | −0.16 | −0.28 |
| F383-0080 | | 61.911 | 62.883 | −0.99 | −2.05 | −0.11 | −0.14 |
| D727-0535 | | 63.743 | 56.828 | −0.99 | −2.07 | −0.14 | −0.22 |
| D727-0404 | | 63.741 | 53.633 | −0.99 | −3.13 | −0.14 | −0.34 |
| G856-9836 | | 73.979 | 71.928 | −0.99 | −4.76 | −0.10 | −0.21 |
| cpd182 | | 51.29 | 54.457 | −0.99 | −1.37 | −0.17 | −0.28 |
| F321-0906 | | 61.889 | 63.685 | −1.00 | −1.56 | −0.11 | −0.11 |
| cpd142 | | 51.267 | 57.833 | −1.00 | −0.77 | −0.17 | −0.16 |
| G946-0405 | | 73.95 | 73.707 | −1.01 | −2.63 | −0.10 | −0.11 |
| T828-0650 | | 73.053 | 70.969 | −1.03 | −2.69 | −0.16 | −0.28 |
| D727-0348 | | 63.593 | 58.679 | −1.04 | −1.46 | −0.14 | −0.16 |
| D727-0754 | | 63.517 | 46.376 | −1.06 | −5.54 | −0.15 | −0.59 |
| D727-0837 | | 63.496 | 59.923 | −1.06 | −1.05 | −0.15 | −0.11 |
| G775-0641 | | 73.8 | 75.898 | −1.09 | 0.01 | −0.10 | 0.00 |
| cpd170 | | 50.722 | 50.376 | −1.09 | −2.09 | −0.18 | −0.42 |
| M040-0939 | | 72.858 | 72.208 | −1.09 | −2.07 | −0.17 | −0.22 |
| D727-0491 | | 63.388 | 57.686 | −1.10 | −1.79 | −0.15 | −0.19 |
| cpd125 | | 50.683 | 53.193 | −1.10 | −1.59 | −0.18 | −0.32 |
| L262-0843 | | 73.779 | 73.266 | −1.10 | −3.16 | −0.11 | −0.14 |
| cpd179 | | 50.632 | 57.994 | −1.11 | −0.74 | −0.18 | −0.15 |
| M144-0886 | | 72.811 | 74.415 | −1.11 | −0.95 | −0.17 | −0.10 |
| D727-0712 | | 63.284 | 55.062 | −1.13 | −2.66 | −0.16 | −0.29 |
| F324-0137 | | 61.485 | 62.997 | −1.14 | −1.98 | −0.12 | −0.13 |
| Cpd17 | | 54.913 | 53.875 | −1.14 | −0.77 | −0.08 | −0.04 |
| E722-2588 | | 63.246 | 66.773 | −1.14 | 1.22 | −0.16 | 0.13 |
| M040-0194 | | 72.713 | 72.563 | −1.14 | −1.89 | −0.18 | −0.20 |
| Cpd54 | | 54.896 | 53.137 | −1.15 | −1.17 | −0.08 | −0.06 |
| Cpd22 | | 54.881 | 57.194 | −1.15 | 1.02 | −0.08 | 0.05 |
| SAM001246592 | | 72.687 | 71.988 | −1.15 | −2.18 | −0.18 | −0.23 |
| D727-0884 | | 63.178 | 56.742 | −1.16 | −2.10 | −0.16 | −0.23 |
| D727-0536 | | 63.106 | 60.482 | −1.18 | −0.86 | −0.16 | −0.09 |
| Cpd69 | | 54.796 | 52.286 | −1.18 | −1.63 | −0.08 | −0.08 |
| G947-4580 | | 73.616 | 69.621 | −1.19 | −7.54 | −0.11 | −0.33 |
| Cpd15 | | 54.778 | 50.847 | −1.19 | −2.41 | −0.08 | −0.12 |
| F086-0004 | | 63.051 | 54.937 | −1.20 | −2.70 | −0.17 | −0.29 |
| F083-0426 | | 63.041 | 59.839 | −1.20 | −1.08 | −0.17 | −0.12 |
| Cpd53 | | 54.731 | 51.141 | −1.21 | −2.25 | −0.08 | −0.11 |
| L942-0265 | | 72.441 | 71.685 | 1.24 | −2.33 | −0.19 | −0.24 |
| D727-0890 | | 62.913 | 57.037 | −1.24 | −2.01 | −0.17 | −0.22 |
| J065-2258 | | 73.517 | 74.702 | −1.25 | −1.43 | −0.12 | −0.06 |
| F518-0013 | | 61.162 | 65.568 | −1.25 | −0.42 | −0.14 | −0.03 |
| L662-0668 | | 72.34 | 74.511 | −1.27 | −0.90 | −0.19 | −0.09 |
| cpd114 | | 49.659 | 60.881 | −1.28 | −0.23 | −0.21 | −0.05 |
| J030-0069 | | 73.451 | 74.359 | −1.28 | −1.84 | −0.12 | −0.08 |
| cpd120 | | 49.616 | 60.254 | −1.29 | −0.34 | −0.22 | −0.07 |
| F293-0962 | | 62.732 | 53.726 | −1.30 | −3.10 | −0.18 | −0.33 |
| cpd183 | | 49.557 | 53.899 | −1.30 | −1.47 | −0.22 | −0.30 |
| G946-0483 | | 73.418 | 76.86 | −1.30 | 1.16 | −0.13 | 0.05 |
| P615-0091 | | 72.248 | 71.013 | −1.30 | −2.67 | −0.20 | −0.28 |
| F518-0002 | | 61.012 | 57.978 | −1.31 | −5.03 | −0.14 | −0.34 |
| J024-0550 | | 73.392 | 73.551 | −1.31 | −2.81 | −0.13 | −0.12 |
| F325-0581 | | 60.956 | 59.005 | −1.32 | −4.41 | −0.14 | −0.30 |
| Cpd78 | | 54.421 | 51.899 | −1.33 | −1.84 | −0.09 | −0.09 |
| cpd192 | | 49.374 | 49.374 | −1.33 | −2.27 | −0.22 | −0.46 |
| Cpd50 | | 54.413 | 60.329 | −1.33 | 2.71 | −0.09 | 0.13 |
| G881-0290 | | 73.358 | 74.897 | −1.33 | −1.20 | −0.13 | −0.05 |
| F128-0043 | | 62.601 | 58.785 | −1.34 | −1.43 | −0.18 | −0.15 |
| F516-0001 | | 60.916 | 63.852 | −1.34 | −1.46 | −0.14 | −0.10 |
| G786-0299 | | 73.346 | 76.271 | −1.34 | 0.46 | −0.13 | 0.02 |
| F321-0610 | | 60.89 | 65.024 | −1.35 | −0.75 | −0.15 | −0.05 |
| D727-0794 | | 62.524 | 56.312 | −1.36 | −2.25 | −0.19 | −0.24 |
| E613-0091 | | 62.484 | 54.219 | −1.37 | −2.94 | −0.19 | −0.32 |
| G786-1280 | | 73.263 | 74.851 | −1.39 | −1.25 | −0.13 | −0.05 |
| F293-0183 | | 62.398 | 53.8 | −1.40 | −3.08 | −0.19 | −0.33 |
| F294-0900 | | 62.393 | 56.357 | −1.40 | −2.23 | −0.19 | −0.24 |
| F518-0029 | | 60.74 | 58.786 | −1.40 | −4.54 | −0.15 | −0.31 |
| Cpd19 | | 54.236 | 60.685 | −1.40 | 2.90 | −0.10 | 0.14 |
| L662-0660 | | 71.954 | 72.822 | −1.40 | −1.76 | −0.21 | −0.18 |
| cpd162 | | 48.961 | 48.353 | −1.41 | −2.45 | −0.23 | −0.50 |
| cpd191 | | 48.816 | 48.816 | −1.43 | −2.37 | −0.24 | −0.48 |
| Cpd6 | | 54.15 | 57.456 | −1.44 | 1.16 | −0.10 | 0.06 |
| G786-0334 | | 60.631 | 58.371 | −1.44 | −4.79 | −0.16 | −0.32 |
| cpd177 | | 48.545 | 50.902 | −1.48 | −2.00 | −0.25 | −0.40 |
| F086-0030 | | 62.068 | 58.34 | −1.50 | −1.57 | −0.21 | −0.17 |
| G786-1567 | | 60.446 | 61.527 | −1.50 | −2.87 | −0.16 | −0.19 |
| F293-0009 | | 62.037 | 61.206 | −1.51 | −0.62 | −0.21 | −0.07 |
| G881-0499 | | 73.039 | 73.122 | −1.51 | −3.33 | −0.15 | −0.15 |
| F512-0802 | | 60.421 | 55.178 | −1.51 | −6.73 | −0.16 | −0.45 |
| F083-0315 | | 61.99 | 60.059 | −1.52 | −1.00 | −0.21 | −0.11 |
| L662-0325 | | 71.58 | 69.646 | −1.53 | −3.36 | −0.23 | −0.35 |
| F358-0116 | | 60.366 | 52.084 | −1.53 | −8.61 | −0.17 | −0.58 |
| G947-0077 | | 72.972 | 73.86 | −1.55 | −2.44 | −0.15 | −0.11 |
| J094-0187 | | 72.97 | 70.829 | −1.55 | −6.08 | −0.15 | −0.27 |
| G554-0497 | | 60.314 | 57.678 | −1.55 | −5.21 | −0.17 | −0.35 |
| Cpd61 | | 53.822 | 55.091 | −1.56 | −0.12 | −0.11 | −0.01 |
| J075-2706 | | 72.936 | 74.775 | −1.57 | −1.34 | −0.15 | −0.06 |
| G779-0144 | | 72.931 | 74.834 | −1.57 | −1.27 | −0.15 | −0.06 |
| F128-0042 | | 61.817 | 52.985 | −1.57 | −3.35 | −0.22 | −0.36 |
| D727-0879 | | 61.798 | 62.743 | −1.58 | −0.11 | −0.22 | −0.01 |
| F516-0012 | | 60.236 | 56.883 | −1.58 | −5.70 | −0.17 | −0.38 |
| F083-0007 | | 61.717 | 59.2 | −1.60 | −1.29 | −0.22 | −0.14 |
| F387-0925 | | 60.155 | 58.828 | −1.61 | −4.51 | −0.17 | −0.30 |
| F313-4535 | | 60.132 | 61.208 | −1.62 | −3.07 | −0.17 | −0.21 |
| F083-0404 | | 61.666 | 49.573 | −1.62 | −4.48 | −0.22 | −0.48 |
| G771-0002 | | 72.823 | 73.311 | −1.63 | −3.10 | −0.16 | −0.14 |
| cpd126 | | 47.687 | 56.011 | −1.63 | −1.09 | −0.27 | −0.22 |
| Cpd51 | | 53.633 | 48.988 | −1.64 | −3.41 | −0.11 | −0.16 |
| D727-0878 | | 61.604 | 57.513 | −1.64 | −1.85 | −0.23 | −0.20 |
| G784-0087 | | 72.795 | 77.51 | −1.64 | 1.95 | −0.16 | 0.09 |
| F379-0115 | | 60.049 | 57.778 | −1.64 | −5.15 | −0.18 | −0.35 |
| F083-0005 | | 61.541 | 59.801 | −1.66 | −1.09 | −0.23 | −0.12 |
| G786-0344 | | 72.767 | 72.552 | −1.66 | −4.01 | −0.16 | −0.18 |
| D727-0355 | | 61.523 | 61.088 | −1.66 | −0.66 | −0.23 | −0.07 |
| F517-0130 | | 59.989 | 58.733 | −1.67 | −4.57 | −0.18 | −0.31 |
| M040-0039 | | 71.147 | 72.167 | −1.68 | −2.09 | −0.26 | −0.22 |
| F386-0042 | | 59.926 | 65.004 | −1.69 | −0.76 | −0.18 | −0.05 |
| Cpd83 | | 53.489 | 47.56 | −1.69 | −4.18 | −0.12 | −0.20 |
| L663-1002 | | 71.095 | 76.396 | −1.70 | 0.05 | −0.26 | 0.01 |
| F516-0016 | | 59.888 | 61.381 | −1.70 | −2.96 | −0.18 | −0.20 |
| J015-0213 | | 72.67 | 74.631 | −1.71 | −1.52 | −0.17 | −0.07 |
| Cpd28 | | 53.433 | 59.645 | −1.71 | 2.34 | −0.12 | 0.11 |
| J075-3081 | | 72.662 | 70.864 | −1.72 | −6.04 | −0.17 | −0.26 |
| L662-0607 | | 71.034 | 75.553 | −1.72 | −0.38 | −0.26 | −0.04 |
| G775-0674 | | 59.816 | 50.746 | −1.73 | −9.42 | −0.19 | −0.63 |
| F323-0058 | | 59.814 | 56.359 | −1.73 | −6.01 | −0.19 | −0.40 |
| SAM001247065 | | 71.005 | 64.458 | −1.73 | −5.98 | −0.26 | −0.62 |
| Cpd92 | | 53.379 | 43.469 | −1.73 | −6.39 | −0.12 | −0.31 |
| K261-1443 | | 72.619 | 72.337 | −1.74 | −4.27 | −0.17 | −0.19 |
| G786-1254 | | 59.719 | 60.309 | −1.76 | −3.61 | −0.19 | −0.24 |
| F521-0014 | | 59.713 | 58.978 | −1.76 | −4.42 | −0.19 | −0.30 |
| D727-0489 | | 61.183 | 61.347 | −1.76 | −0.58 | −0.24 | −0.06 |
| D727-0088 | | 61.161 | 60.83 | −1.77 | −0.75 | −0.24 | −0.08 |
| G786-1481 | | 72.562 | 72.568 | −1.77 | −3.99 | −0.17 | −0.17 |
| cpd190 | | 46.867 | 46.867 | −1.78 | −2.72 | −0.30 | −0.55 |
| F083-0285 | | 61.126 | 49.642 | −1.78 | −4.46 | −0.25 | −0.48 |
| F516-0005 | | 59.656 | 56.929 | −1.78 | −5.67 | −0.19 | −0.38 |
| D727-0502 | | 61.043 | 59.799 | −1.81 | −1.09 | −0.25 | −0.12 |
| F378-0422 | | 59.586 | 57.787 | −1.81 | −5.15 | −0.20 | −0.35 |
| J026-0004 | | 72.497 | 73.123 | −1.81 | −3.33 | −0.17 | −0.15 |
| G771-0448 | | 59.569 | 59.858 | −1.81 | −3.89 | −0.20 | −0.26 |
| D727-0339 | | 60.982 | 58.84 | −1.83 | −1.41 | −0.25 | −0.15 |
| cpd136 | | 46.552 | 53.626 | −1.83 | −1.52 | −0.31 | −0.31 |
| G786-0351 | | 72.45 | 74.962 | −1.84 | −1.12 | −0.18 | −0.05 |
| G881-0295 | | 72.43 | 72.433 | −1.85 | −4.16 | −0.18 | −0.18 |
| cpd130 | | 46.44 | 49.679 | −1.85 | −2.22 | −0.31 | −0.45 |
| cpd159 | | 46.304 | 60.888 | −1.88 | −0.23 | −0.31 | −0.05 |
| F086-0033 | | 60.787 | 56.359 | −1.88 | −2.23 | −0.26 | −0.24 |
| G947-4440 | | 72.339 | 74.051 | −1.90 | −2.21 | −0.18 | −0.10 |
| G769-1003 | | 59.326 | 58.884 | −1.90 | −4.48 | −0.21 | −0.30 |
| Cpd47 | | 52.909 | 54.973 | −1.92 | −0.18 | −0.13 | −0.01 |
| F281-0079 | | 60.677 | 57.847 | −1.92 | −1.74 | −0.27 | −0.19 |
| M040-0203 | | 70.426 | 73.177 | −1.93 | −1.58 | −0.29 | −0.16 |
| L065-0741 | | 72.267 | 73.491 | −1.94 | −2.89 | −0.19 | −0.13 |
| cpd101 | | 45.981 | 64.578 | −1.94 | 0.43 | −0.32 | 0.09 |
| D727-0915 | | 60.546 | 58.834 | −1.96 | −1.41 | −0.27 | −0.15 |
| F518-0008 | | 59.029 | 61.293 | −2.00 | −3.02 | −0.22 | −0.20 |
| Cpd76 | | 52.679 | 49.721 | −2.01 | −3.01 | −0.14 | −0.15 |
| F321-0507 | | 59.013 | 60.043 | −2.01 | −3.78 | −0.22 | −0.25 |
| D727-0853 | | 60.361 | 59.417 | −2.01 | −1.22 | −0.28 | −0.13 |
| cpd116 | | 45.496 | 62.821 | −2.02 | 0.12 | −0.34 | 0.02 |
| G786-1316 | | 58.95 | 60.05 | −2.03 | −3.77 | −0.22 | −0.25 |
| D727-0892 | | 60.296 | 53.657 | −2.03 | −3.13 | −0.28 | −0.34 |
| D727-0025 | | 60.269 | 59.908 | −2.04 | −1.05 | −0.28 | −0.11 |
| F521-0258 | | 58.905 | 56.798 | −2.05 | −5.75 | −0.22 | −0.39 |
| F324-0189 | | 58.787 | 58.946 | −2.09 | −4.44 | −0.23 | −0.30 |
| Cpd62 | | 52.448 | 46.754 | −2.10 | −4.61 | −0.15 | −0.22 |
| F896-0460 | | 58.749 | 58.686 | −2.10 | −4.60 | −0.23 | −0.31 |
| F512-0173 | | 58.65 | 54.46 | −2.14 | −7.17 | −0.23 | −0.48 |
| F323-0007 | | 58.589 | 59.11 | −2.16 | −4.34 | −0.23 | −0.29 |
| G520-0047 | | 58.575 | 56.795 | −2.16 | −5.75 | −0.23 | −0.39 |
| D727-0202 | | 59.812 | 62.001 | −2.18 | −0.36 | −0.30 | −0.04 |
| Cpd81 | | 52.196 | 51.492 | −2.19 | −2.06 | −0.15 | −0.10 |
| F086-0029 | | 59.751 | 61.703 | −2.20 | −0.46 | −0.30 | −0.05 |
| G935-2190 | | 71.773 | 71.841 | −2.21 | −4.87 | −0.21 | −0.21 |
| E676-2021 | | 59.698 | 56.018 | −2.21 | −2.34 | −0.31 | −0.25 |
| Cpd7 | | 52.143 | 50.699 | −2.22 | −2.49 | −0.16 | −0.12 |
| F387-1175 | | 58.408 | 58.893 | −2.22 | −4.47 | −0.24 | −0.30 |
| F512-0190 | | 58.405 | 56.062 | −2.22 | −6.19 | −0.24 | −0.42 |
| Cpd95 | | 52.074 | 53.576 | −2.24 | −0.93 | −0.16 | −0.05 |
| F378-0505 | | 58.315 | 49.443 | −2.26 | −10.21 | −0.24 | −0.69 |
| D727-0069 | | 59.543 | 58.398 | −2.26 | −1.55 | −0.31 | −0.17 |
| Cpd60 | | 52.003 | 54.856 | −2.27 | −0.24 | −0.16 | −0.01 |
| Cpd25 | | 52 | 46.791 | −2.27 | −4.59 | −0.16 | −0.22 |
| F083-0416 | | 59.472 | 61.595 | −2.28 | −0.49 | −0.32 | −0.05 |
| G775-0454 | | 71.632 | 74.022 | −2.29 | −2.25 | −0.22 | −0.10 |
| F378-0537 | | 58.196 | 59.806 | −2.30 | −3.92 | −0.25 | −0.26 |
| G786-1561 | | 71.564 | 72.223 | −2.32 | −4.41 | −0.22 | −0.19 |
| F128-0049 | | 59.294 | 57.219 | −2.34 | −1.95 | −0.32 | −0.21 |
| G933-0087 | | 71.541 | 70.48 | −2.34 | −6.50 | −0.23 | −0.28 |
| F305-0129 | | 59.262 | 52.541 | −2.35 | −3.50 | −0.32 | −0.38 |
| F343-0097 | | 58.054 | 60.645 | −2.35 | −3.41 | −0.25 | −0.23 |
| F294-0183 | | 59.182 | 54.549 | −2.37 | −2.83 | −0.33 | −0.30 |
| F516-0003 | | 57.986 | 57.569 | −2.37 | −5.28 | −0.26 | −0.35 |
| F324-0076 | | 57.977 | 60.823 | −2.38 | −3.30 | −0.26 | −0.22 |
| F322-0903 | | 57.964 | 58.681 | −2.38 | −4.60 | −0.26 | −0.31 |
| G948-5129 | | 71.454 | 71.807 | −2.38 | −4.91 | −0.23 | −0.21 |
| Cpd80 | | 51.658 | 55.1 | −2.40 | −0.11 | −0.17 | −0.01 |
| G786-1562 | | 57.872 | 60.132 | −2.41 | −3.72 | −0.26 | −0.25 |
| G784-0129 | | 71.385 | 76.422 | −2.42 | 0.64 | −0.23 | 0.03 |
| F863-0112 | | 57.824 | 60.779 | −2.43 | −3.33 | −0.26 | −0.22 |
| F685-1588 | | 57.803 | 60.73 | −2.44 | −3.36 | −0.26 | −0.23 |
| G786-0389 | | 71.337 | 73.301 | −2.45 | −3.11 | −0.24 | −0.14 |
| F083-0012 | | 58.875 | 58.674 | −2.46 | −1.46 | −0.34 | −0.16 |
| F324-0080 | | 57.72 | 59.715 | −2.47 | −3.98 | −0.27 | −0.27 |
| D727-0047 | | 58.846 | 54.891 | −2.47 | −2.72 | −0.34 | −0.29 |
| F378-0208 | | 57.692 | 57.682 | −2.48 | −5.21 | −0.27 | −0.35 |
| Cpd8 | | 51.455 | 54.983 | −2.48 | −0.17 | −0.17 | −0.01 |
| G775-0370 | | 71.273 | 74.401 | −2.48 | −1.79 | −0.24 | −0.08 |
| F512-1035 | | 57.633 | 55.8 | −2.50 | −6.35 | −0.27 | −0.43 |
| G933-0058 | | 71.21 | 73.948 | −2.52 | −2.34 | −0.24 | −0.10 |
| F514-0637 | | 57.561 | 59.332 | −2.52 | −4.21 | −0.27 | −0.28 |
| G937-0468 | | 71.134 | 70.134 | −2.56 | −6.92 | −0.25 | −0.30 |
| F321-0021 | | 58.481 | 44.991 | −2.58 | −6.00 | −0.36 | −0.64 |
| G786-1482 | | 71.046 | 74.029 | −2.61 | −2.24 | −0.25 | −0.10 |
| D727-0828 | | 58.376 | 59.045 | −2.61 | −1.34 | −0.36 | −0.14 |
| F293-0616 | | 58.357 | 62.171 | −2.62 | −0.30 | −0.36 | −0.03 |
| F322-0863 | | 57.241 | 55.709 | −2.64 | −6.41 | −0.29 | −0.43 |
| F325-0062 | | 57.197 | 65.694 | −2.65 | −0.34 | −0.29 | −0.02 |
| G774-0218 | | 57.196 | 60.92 | −2.65 | −3.24 | −0.29 | −0.22 |
| F379-0058 | | 57.155 | 61.018 | −2.67 | −3.18 | −0.29 | −0.21 |
| F321-0902 | | 57.152 | 58.475 | −2.67 | −4.73 | −0.29 | −0.32 |
| G947-0023 | | 70.9 | 71.089 | −2.69 | −5.77 | −0.26 | −0.25 |
| L378-0331 | | 68.157 | 77.513 | −2.70 | 0.61 | −0.41 | 0.06 |
| F512-0677 | | 57.031 | 59.57 | −2.71 | −4.06 | −0.29 | −0.27 |
| Cpd46 | | 50.834 | 53.703 | −2.72 | −0.87 | −0.19 | −0.04 |
| F385-0601 | | 56.91 | 66.821 | −2.75 | 0.34 | −0.30 | 0.02 |
| Cpd13 | | 50.74 | 51.23 | −2.76 | −2.20 | −0.19 | −0.11 |
| F378-0421 | | 56.876 | 55.452 | −2.76 | −6.56 | −0.30 | −0.44 |
| Cpd73 | | 50.708 | 54.947 | −2.77 | −0.19 | −0.19 | −0.01 |
| F325-0073 | | 56.834 | 58.985 | −2.78 | −4.42 | −0.30 | −0.30 |
| G786-1317 | | 56.787 | 66.138 | −2.80 | −0.07 | −0.30 | 0.00 |
| J015-0222 | | 70.693 | 70.914 | −2.80 | −5.98 | −0.27 | −0.26 |
| F896-0431 | | 56.709 | 57.443 | −2.82 | −5.36 | −0.31 | −0.36 |
| D727-0524 | | 57.663 | 58.659 | −2.83 | −1.47 | −0.39 | −0.16 |
| F324-0038 | | 56.618 | 58.738 | −2.86 | −4.57 | −0.31 | −0.31 |
| F512-0226 | | 56.573 | 57.885 | −2.87 | −5.09 | −0.31 | −0.34 |
| F294-0983 | | 57.443 | 53.339 | −2.90 | −3.23 | −0.40 | −0.35 |
| Cpd52 | | 50.233 | 49.557 | −2.96 | −3.10 | −0.21 | −0.15 |
| Cpd89 | | 50.218 | 57.771 | −2.96 | 1.33 | −0.21 | 0.06 |
| G775-0268 | | 56.166 | 60.241 | −3.01 | −3.66 | −0.33 | −0.25 |
| G775-0671 | | 56.138 | 55.132 | −3.02 | −6.76 | −0.33 | −0.45 |
| Cpd93 | | 49.974 | 52.005 | −3.06 | −1.78 | −0.21 | −0.09 |
| F506-0010 | | 55.989 | 52.865 | −3.08 | −8.14 | −0.33 | −0.55 |
| G935-2193 | | 70.167 | 72.724 | −3.09 | −3.81 | −0.30 | −0.17 |
| Cpd77 | | 49.861 | 51.967 | −3.10 | −1.80 | −0.22 | −0.09 |
| F512-0817 | | 55.826 | 53.806 | −3.13 | −7.56 | −0.34 | −0.51 |
| Cpd59 | | 49.745 | 55.582 | −3.15 | 0.15 | −0.22 | 0.01 |
| F324-0150 | | 55.761 | 59.182 | −3.16 | −4.30 | −0.34 | −0.29 |
| Cpd79 | | 49.652 | 57.638 | −3.18 | 1.26 | −0.22 | 0.06 |
| F293-0898 | | 56.403 | 60.078 | −3.21 | −1.00 | −0.44 | −0.11 |
| Cpd32 | | 49.57 | 51.302 | −3.21 | −2.16 | −0.23 | −0.10 |
| D727-0182 | | 56.36 | 67.01 | −3.22 | 1.30 | −0.45 | 0.14 |
| F518-0137 | | 55.554 | 57.507 | −3.23 | −5.32 | −0.35 | −0.36 |
| F516-0008 | | 55.491 | 62.204 | −3.25 | −2.46 | −0.35 | −0.17 |
| J015-0225 | | 69.857 | 72.663 | −3.27 | −3.88 | −0.31 | −0.17 |
| Cpd16 | | 49.436 | 51.872 | −3.27 | −1.85 | −0.23 | −0.09 |
| F518-0014 | | 55.407 | 57.92 | −3.28 | −5.07 | −0.36 | −0.34 |
| Cpd56 | | 49.312 | 55.874 | −3.31 | 0.31 | −0.23 | 0.01 |
| Cpd45 | | 49.274 | 51.411 | −3.33 | −2.10 | −0.23 | −0.10 |
| J030-0068 | | 69.719 | 68.405 | −3.34 | −9.00 | −0.32 | −0.39 |
| G769-1017 | | 55.089 | 57.165 | −3.39 | −5.52 | −0.37 | −0.37 |
| F323-0069 | | 55.065 | 54.849 | −3.40 | −6.93 | −0.37 | −0.47 |
| F372-2527 | | 55.057 | 57.985 | −3.41 | −5.03 | −0.37 | −0.34 |
| D727-0350 | | 55.746 | 61.985 | −3.41 | −0.36 | −0.47 | −0.04 |
| F325-0188 | | 55.029 | 55.443 | −3.42 | −6.57 | −0.37 | −0.44 |
| G775-0475 | | 54.958 | 49.97 | −3.44 | −9.89 | −0.37 | −0.67 |
| F294-0003 | | 55.595 | 60.198 | −3.46 | −0.96 | −0.48 | −0.10 |
| F383-0856 | | 54.899 | 55.355 | −3.46 | −6.62 | −0.37 | −0.45 |
| J015-0243 | | 69.443 | 71.391 | −3.49 | −5.41 | −0.34 | −0.24 |
| Cpd26 | | 48.847 | 49.133 | −3.50 | −3.33 | −0.24 | −0.16 |
| D727-0525 | | 55.263 | 52.257 | −3.56 | −3.59 | −0.49 | −0.39 |
| D727-0201 | | 55.164 | 61.785 | −3.59 | −0.43 | −0.50 | −0.05 |
| Cpd34 | | 48.547 | 51.298 | −3.61 | −2.16 | −0.25 | −0.10 |
| G784-0099 | | 69.21 | 76.066 | −3.62 | 0.21 | −0.35 | 0.01 |
| Cpd4 | | 48.322 | 47.796 | −3.70 | −4.05 | −0.26 | −0.20 |
| G786-1483 | | 54.211 | 59.211 | −3.70 | −4.28 | −0.40 | −0.29 |
| F379-0122 | | 53.847 | 54.162 | −3.83 | −7.35 | −0.41 | −0.49 |
| G786-1569 | | 68.715 | 76.809 | −3.90 | 1.10 | −0.38 | 0.05 |
| F086-0028 | | 53.848 | 57.208 | −3.98 | −1.95 | −0.55 | −0.21 |
| G775-0507 | | 53.279 | 56.412 | −4.03 | −5.98 | −0.44 | −0.40 |
| G771-0900 | | 68.441 | 75.658 | −4.05 | −0.28 | −0.39 | −0.01 |
| D727-0159 | | 53.496 | 56.714 | −4.09 | −2.11 | −0.57 | −0.23 |
| Cpd43 | | 47.313 | 49.654 | −4.09 | −3.05 | −0.29 | −0.15 |
| D727-0740 | | 53.347 | 60.86 | −4.14 | −0.74 | −0.57 | −0.08 |
| G771-1118 | | 52.879 | 52.951 | −4.17 | −8.08 | −0.45 | −0.54 |
| Cpd91 | | 46.621 | 50.304 | −4.36 | −2.70 | −0.31 | −0.13 |
| Cpd14 | | 46.612 | 50.263 | −4.36 | −2.72 | −0.31 | −0.13 |
| Cpd23 | | 46.606 | 54.618 | −4.37 | −0.37 | −0.31 | −0.02 |
| F512-0198 | | 52.253 | 53.985 | −4.40 | −7.46 | −0.48 | −0.50 |
| Cpd88 | | 46.423 | 52.026 | −4.44 | −1.77 | −0.31 | −0.09 |
| E722-1380 | | 51.609 | 61.793 | −4.66 | −0.43 | −0.64 | −0.05 |
| E613-0104 | | 51.441 | 56.456 | −4.71 | −2.20 | −0.65 | −0.24 |
| S2686 | | 62.196 | 65.516 | −4.74 | −5.44 | −0.73 | −0.57 |
| S2743 | | 61.679 | 59.461 | −4.92 | −8.50 | −0.75 | −0.89 |
| Cpd41 | | 43.336 | 52.454 | −5.64 | −1.54 | −0.39 | −0.07 |
| Cpd57 | | 42.071 | 56.986 | −6.13 | 0.91 | −0.43 | 0.04 |
| Cpd3 | | 39.631 | 50.772 | −7.08 | −2.45 | −0.50 | −0.12 |
| Cpd96 | | 39.284 | 33.302 | −7.21 | −11.87 | −0.50 | −0.57 |
| S2621 | | 43.978 | 43.815 | −10.98 | −16.40 | −1.68 | −1.71 |
| SAM001246846 | | 26.655 | 24.765 | −16.90 | −26.02 | −2.59 | −2.71 |
| S2670 | | 10.416 | 9.7985 | −22.46 | −33.58 | −3.44 | −3.50 |
| S2749 | | 0 | 0 | −26.02 | −38.53 | −3.98 | −4.01 |

Hits > 3 s 11
Hits > 2 s 16

Based on the results of the eGFP disruption, preferred SpCas9 inhibitors were identified, Table 3A, Table 3B includes compounds based on performance in eGFP and HiBiT assays.

TABLE 3A
Preferred SpCas9 Inhibitors based on eGFP assay.

| | % GFP+, | % GFP+, | Z score | Z score |
|---|---|---|---|---|
| Compound | Rep 1 | Rep 2 | Rep1 | Rep2 |
| G786-2334 | 99.549501 | 99.675549 | 13.12 | 28.59 |
| G786-2325 | 98.876926 | 98.529701 | 12.75 | 27.21 |
| T5242217 | 82.666548 | 80.630209 | 9.64 | 13.66 |
| G786-1325 | 86.012236 | 86.242625 | 5.65 | 12.44 |
| G786-1572 | 82.405359 | 84.887349 | 3.66 | 10.81 |
| G786-1547 | 82.80155 | 84.08576 | 3.88 | 9.85 |
| G786-1264 | 82.717007 | 83.951389 | 3.83 | 9.69 |
| T5461482 | 66.841744 | 67.113319 | 3.50 | 6.37 |
| T0503-6911 | 67.521537 | 66.588167 | 3.76 | 6.09 |
| T5535170 | 67.964175 | 65.338775 | 3.93 | 5.41 |
| G786-1324 | 81.42696 | 79.939026 | 3.12 | 4.86 |
| T5371551 | 64.27829 | 76.754026 | 2.50 | 11.57 |
| T5264279 | 65.175897 | 67.721763 | 2.85 | 6.70 |
| G946-0488 | 79.391636 | 79.92643 | 2.00 | 4.85 |
| T5213954 | 64.255257 | 63.424341 | 2.49 | 4.38 |
| T0504-2965 | 63.859545 | 62.370813 | 2.34 | 3.81 |

TABLE 3B
SpCas9 Compounds according to performance
in eGFP and HiBiT assays.

| | eGFP | Hibit | |
|---|---|---|---|
| | disruption-% | assay-% | |
| | inhibition | inhibition | Top hits |
| G786-1325 | 56.65 | 97.09 | 1 |
| G786-1324 | 32.94 | 94.5 | 1 |
| G786-2334 | 126.67 | 91.01 | Auto |
| | | | fluorescent |
| T5242217 | 67.52 | 90.84 | Toxic |
| T5451097 | 22.10 | 85.71 | Toxic |
| G786-1264 | 41.72 | 72.63 | 1 |
| T5535170 | 27.53 | 68.25 | 1 |
| 8010-1547 | 32.46 | 57.63 | |
| G786-1572 | 46.53 | 56.61 | Toxic |
| 1927-7835 | 22.13 | 48.79 | |
| F516-0003 | 20.69 | 45.4 | |
| F086-0032 | 24.16 | 37.7 | |
| T5461482 | 24.48 | 36.6 | 1 |
| T0504-1437 | 27.74 | 31.95 | |
| G786-2325 | 123.19 | 29.53 | |
| D727-0165 | 25.03 | 22.01 | |
| F083-0404 | 24.77 | 21.82 | |
| T0503-6911 | 26.33 | 18.06 | |
| D727-0069 | 24.03 | 8.56 | |
| G786-1547 | 42.41 | 4.3 | |
| G946-0488 | 22.41 | 0.9 | |
| T5371551 | 22.88 | −12.66 | |
| T5264279 | 19.95 | −13.06 | |
| D727-0535 | 18.61 | 18.11 | |
| T5213954 | 17.45 | 1.85 | |
| T0504-2965 | 16.37 | −23.8 | |
| G786-0334 | 13.93 | −12.3 | |
| T5280552 | 12.75 | −25.13 | |
| T5385382 | 12.03 | −6.38 | |
| T0515-7259 | 6.16 | 0.49 | |
| T0505-1471 | 5.80 | −25.82 | |
| T5446230 | 5.36 | −7.85 | |
| T5348278 | 4.64 | 16.17 | |
| G786-0265 | −12.20 | 82.96 | |

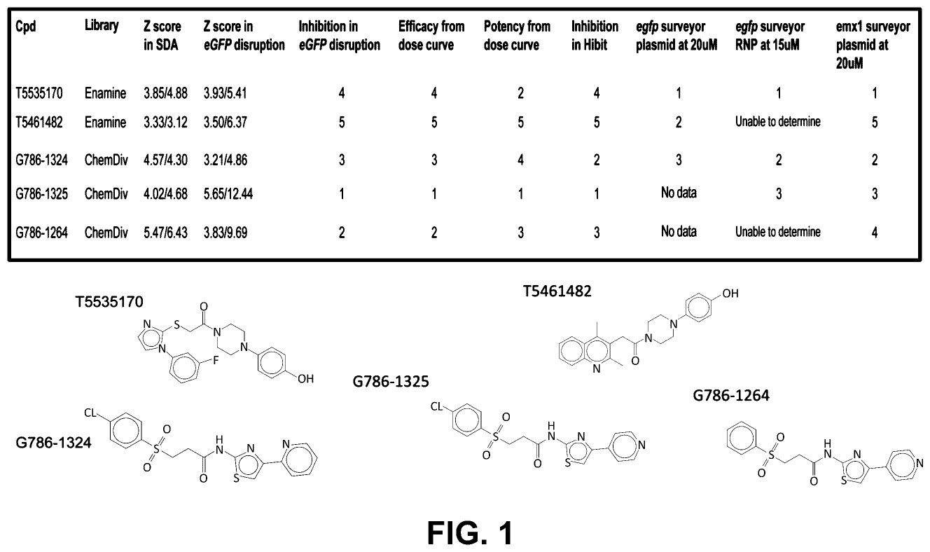

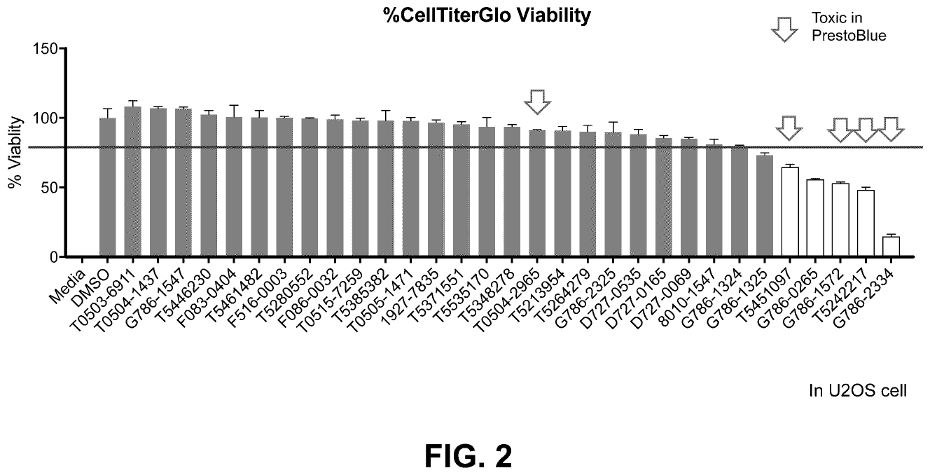

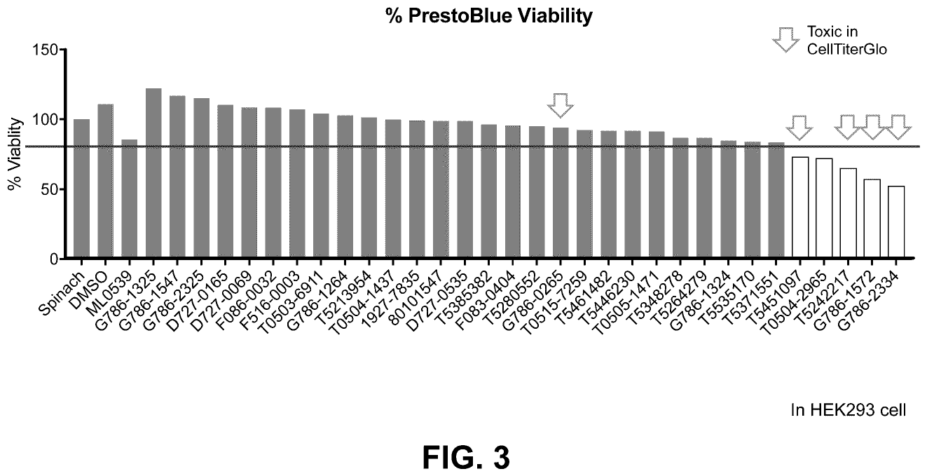

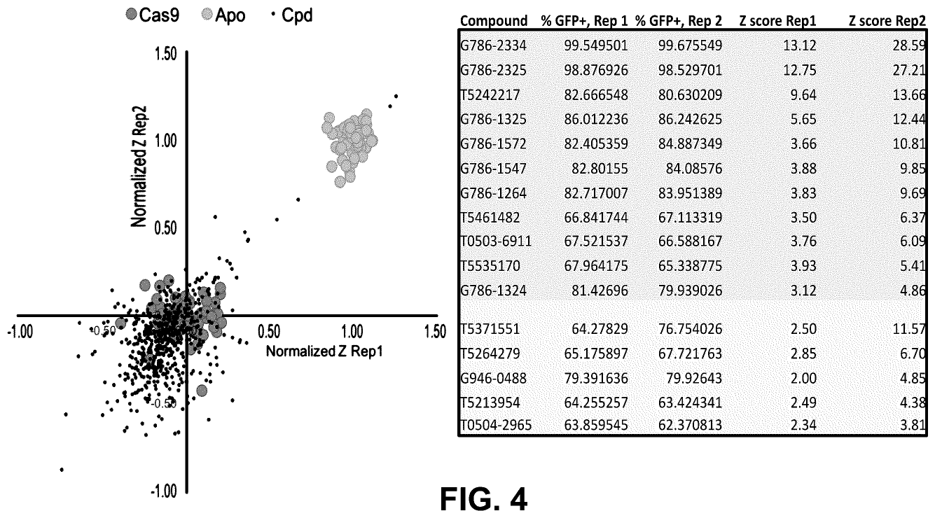

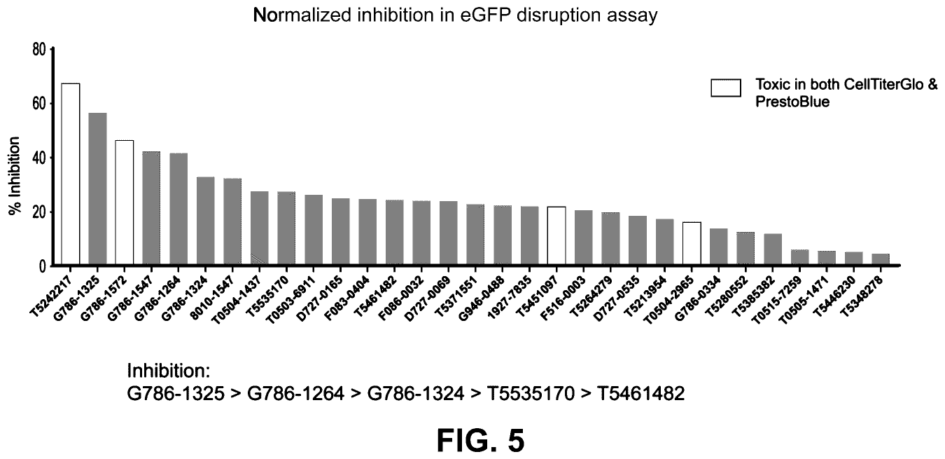

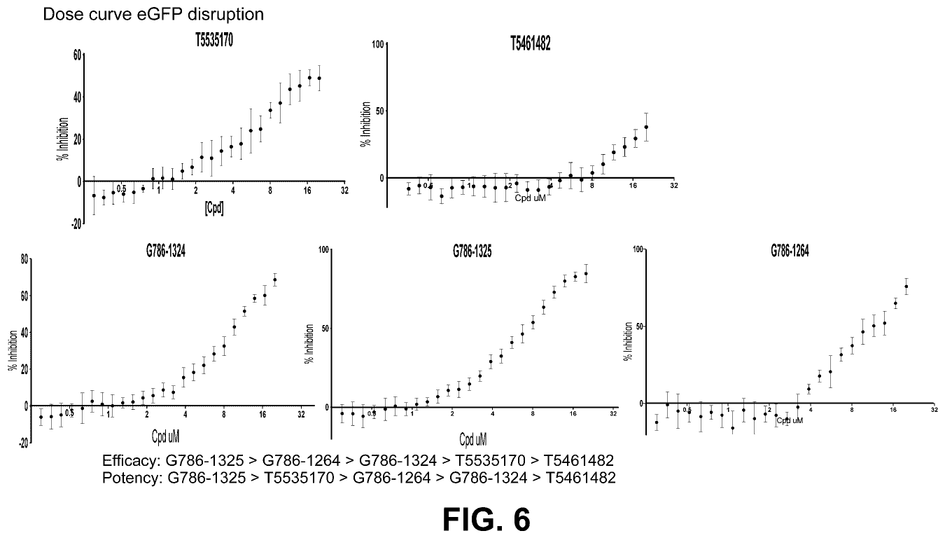

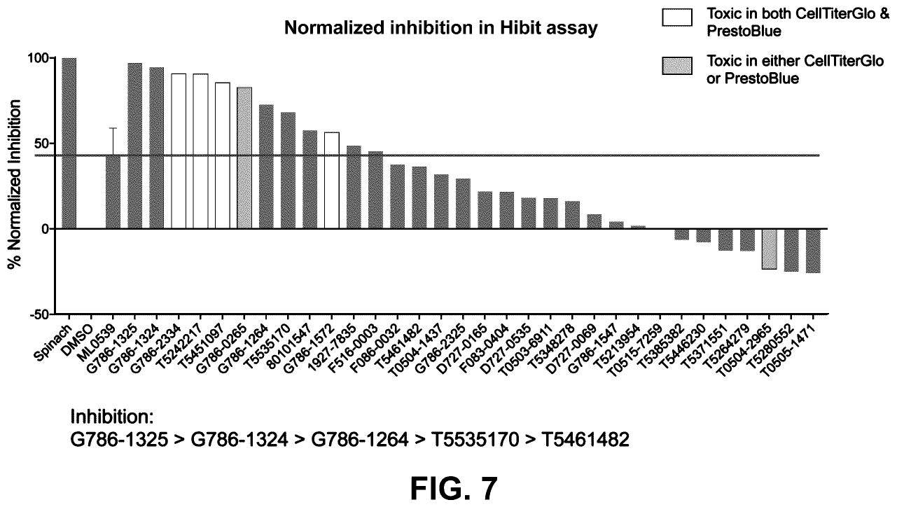

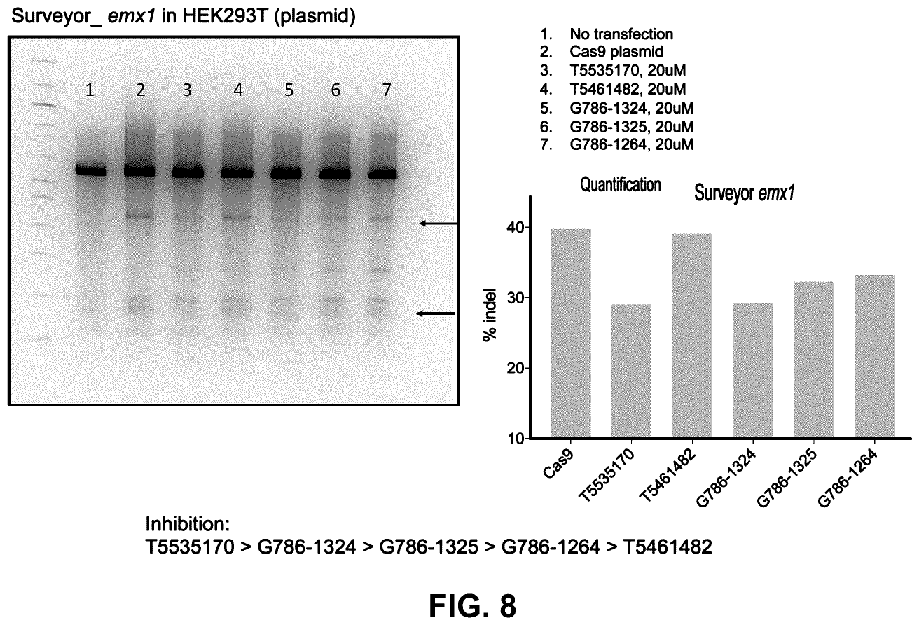

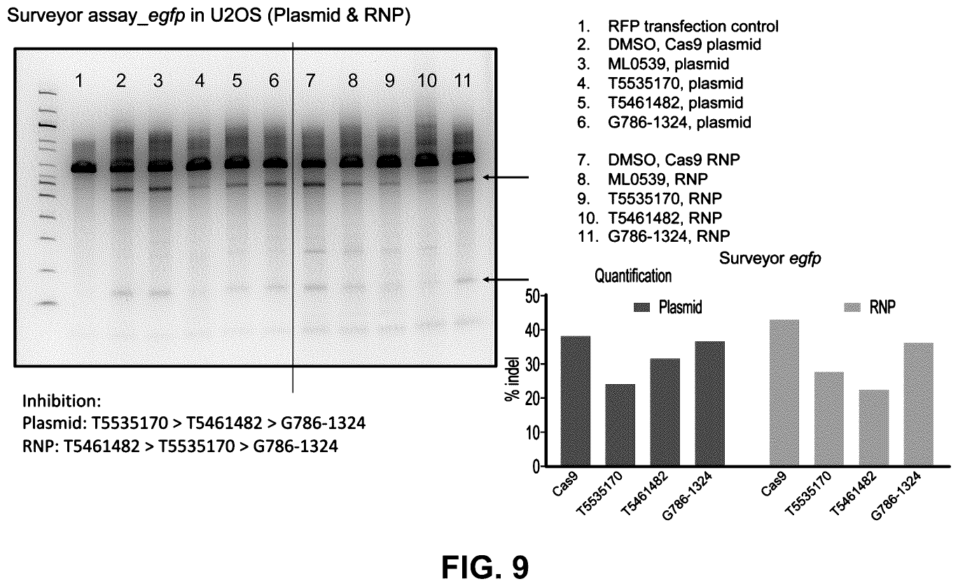

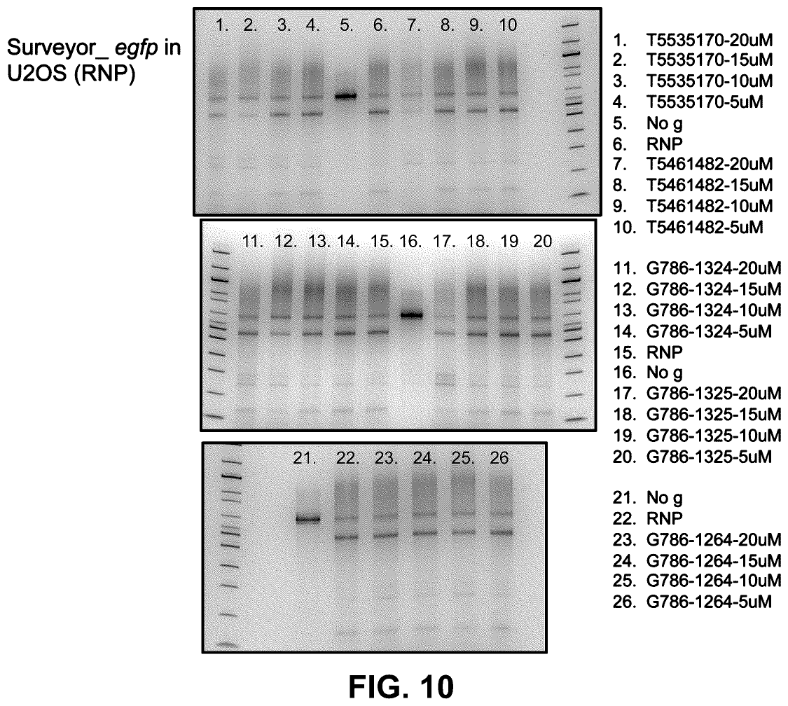

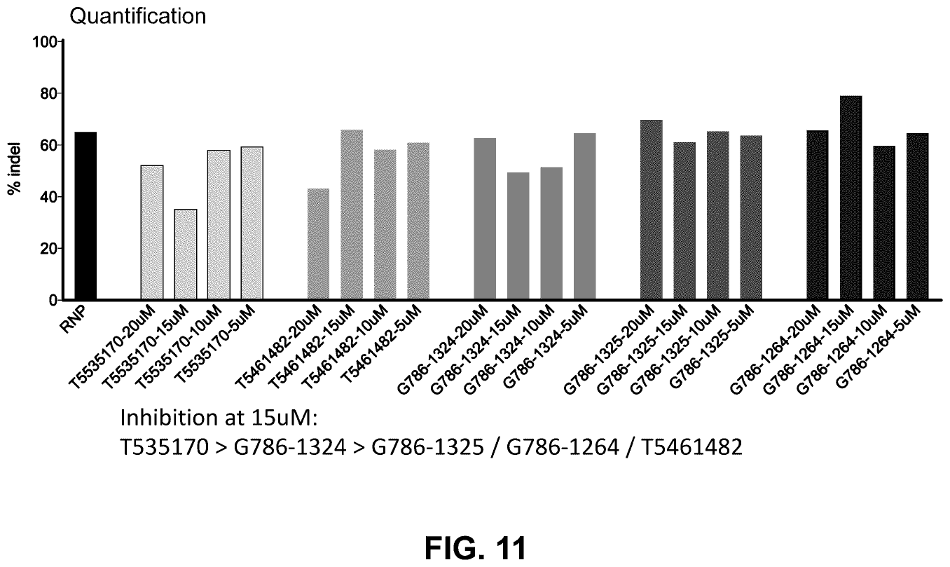

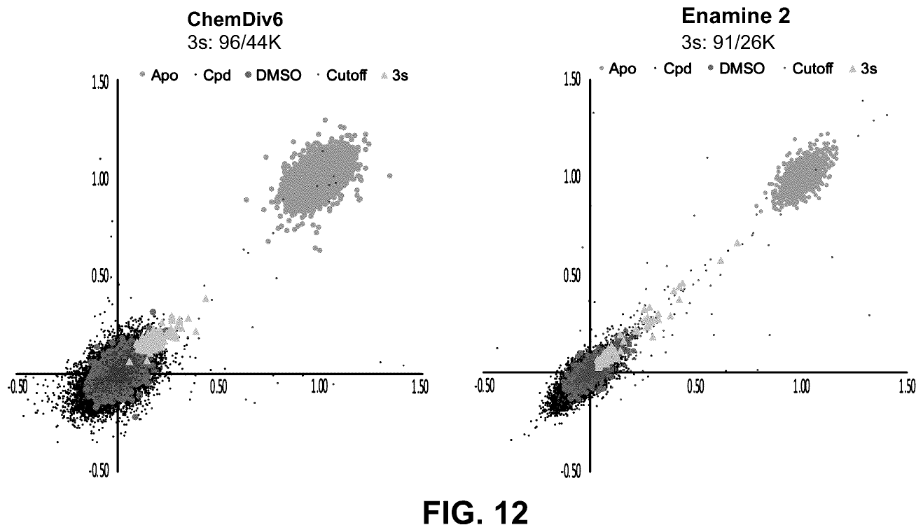

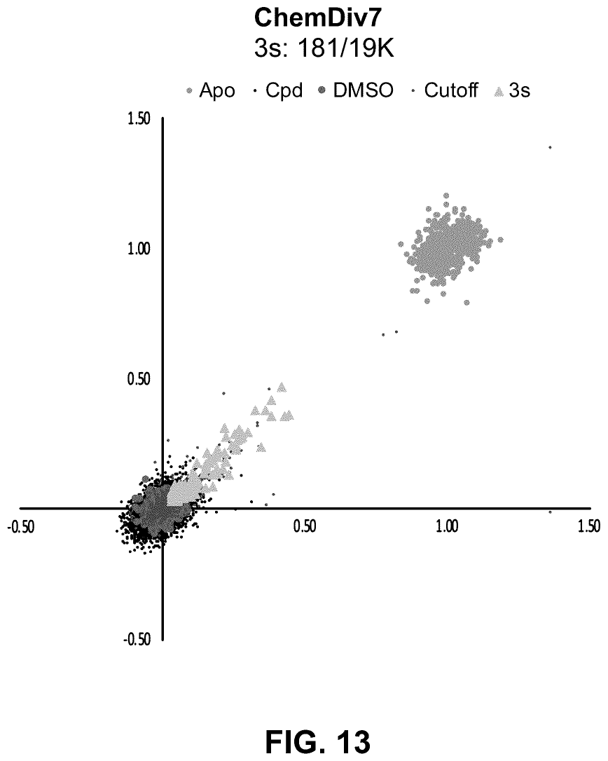

As provided in FIG. 1, several compounds were examined in egfp surveyor assays, both via plasmid and RNP delivery. Preferred compounds include:

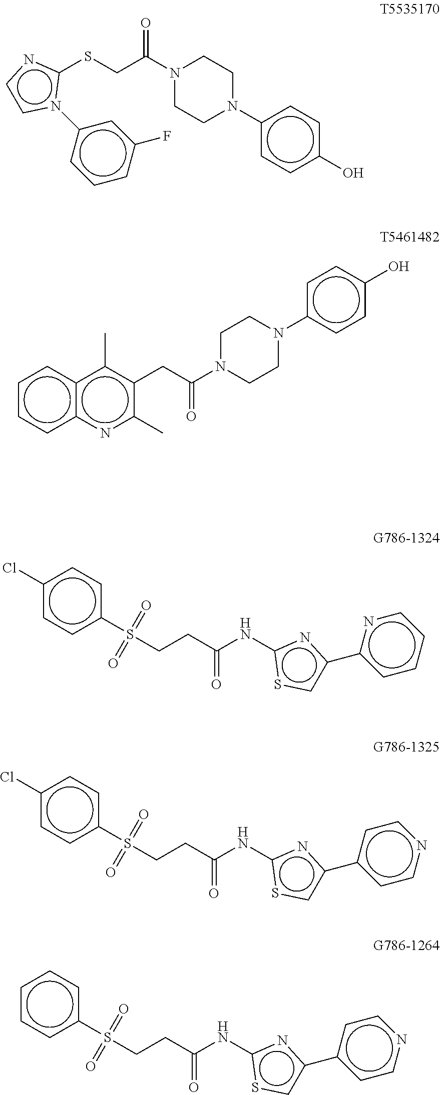

The SpCas9 screening included screening of 149,660 compounds, with total positives of 0.84%. Library screening included Biomol: FDA approved, LOPAC1: pharmacologically active, NCC1-2014: NIH Clinical, Selleck: Bioactive, ChemDiv: Commercially available, Enamine: Commercially available, and Asinex: Commercially available.

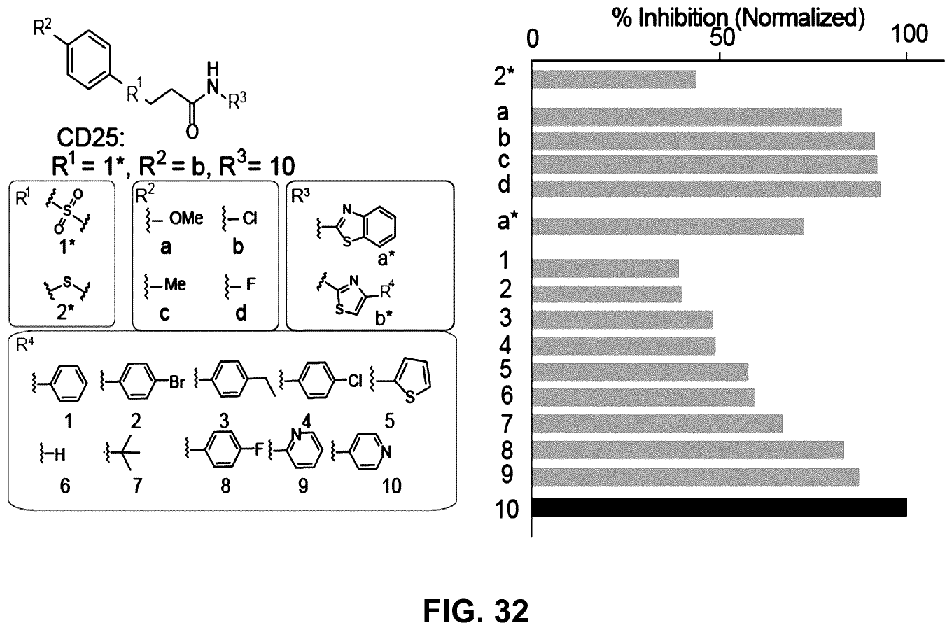

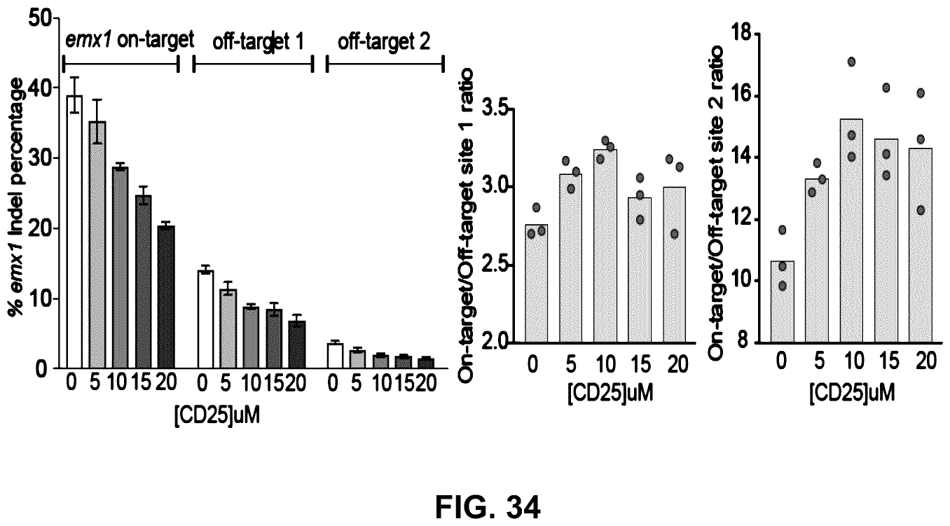

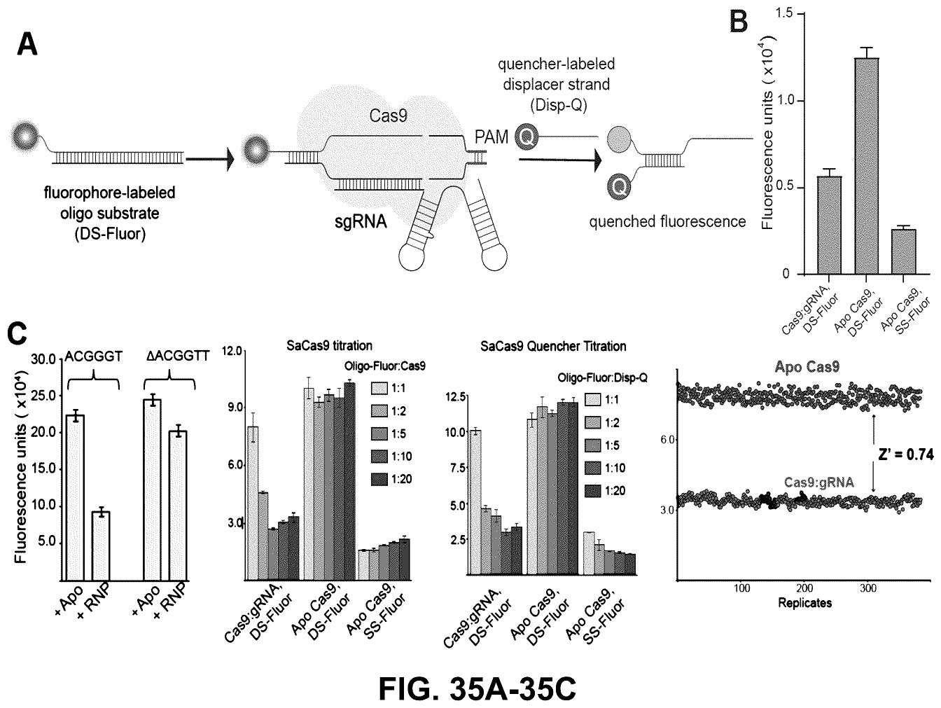

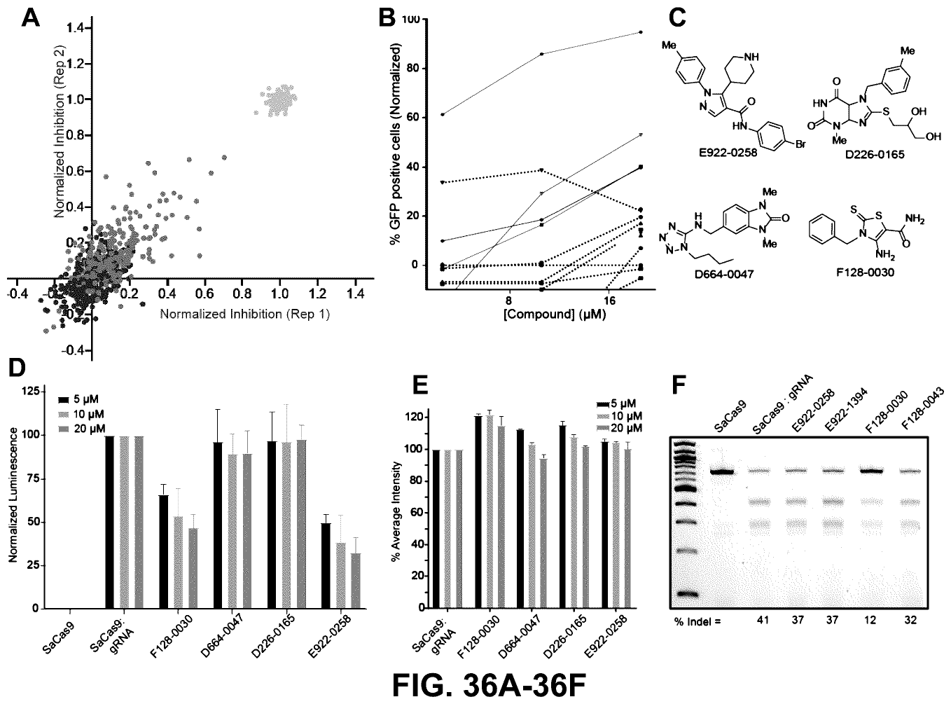

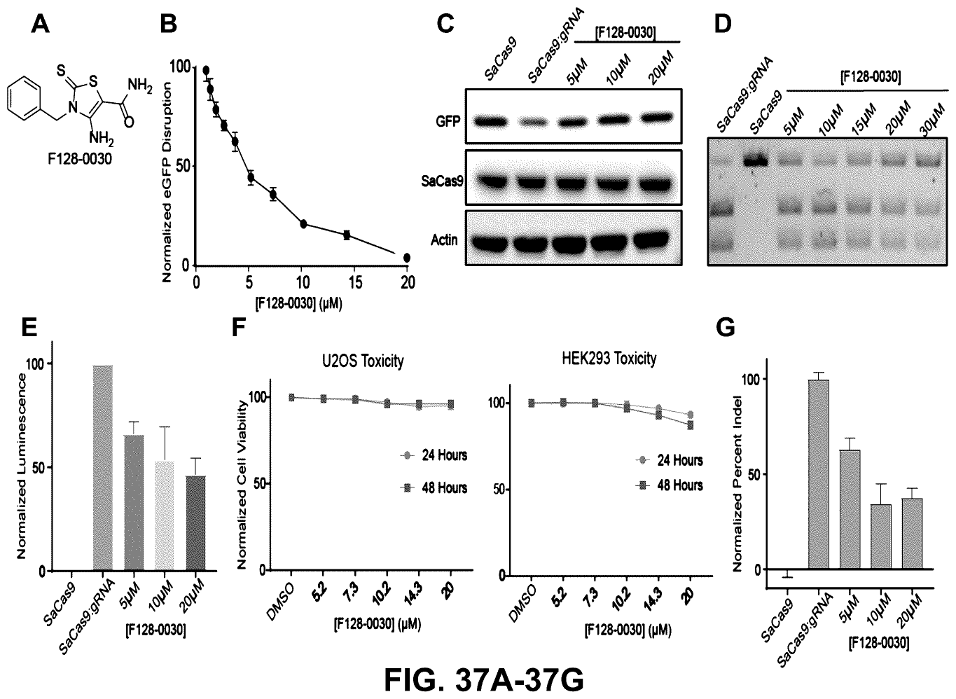

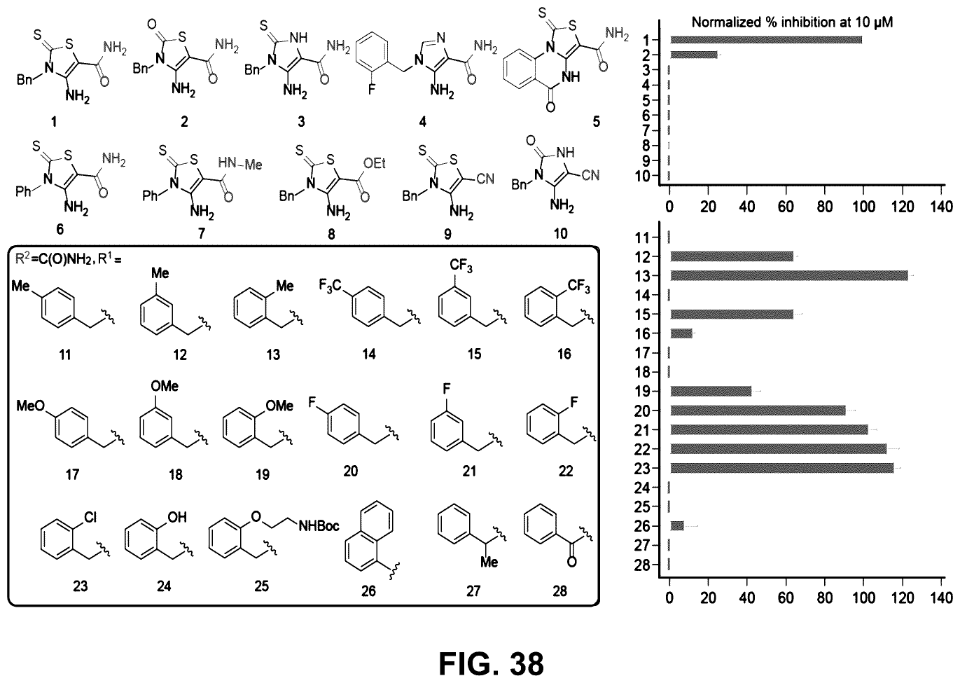

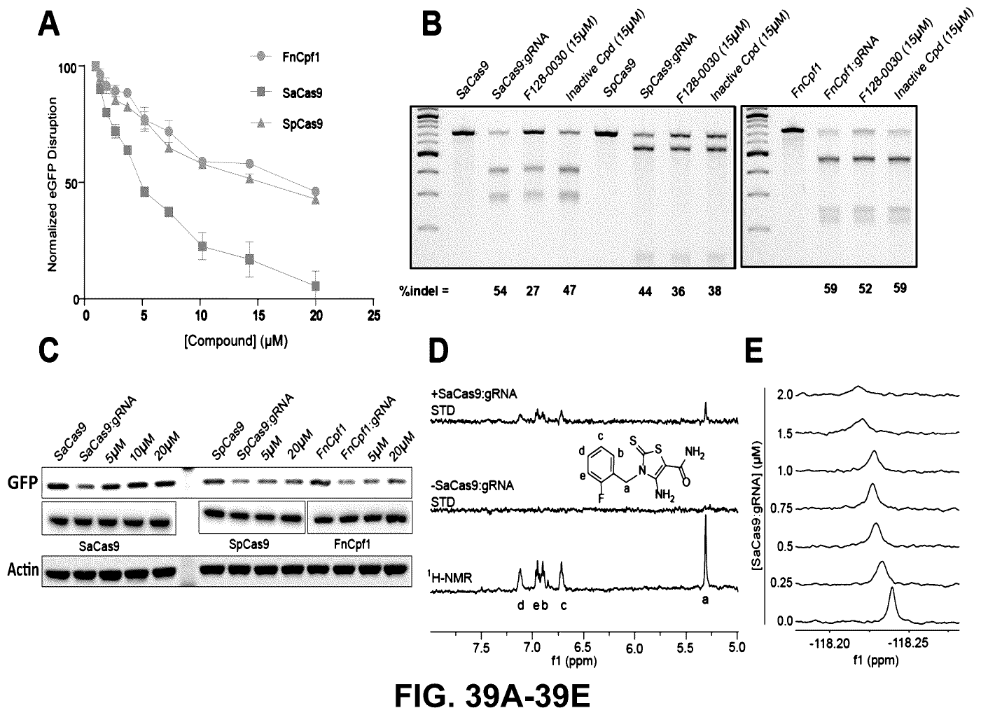

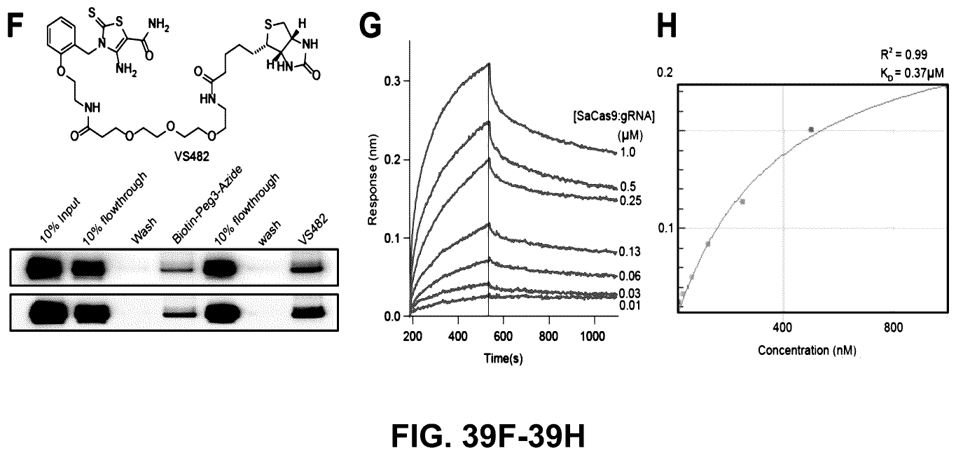

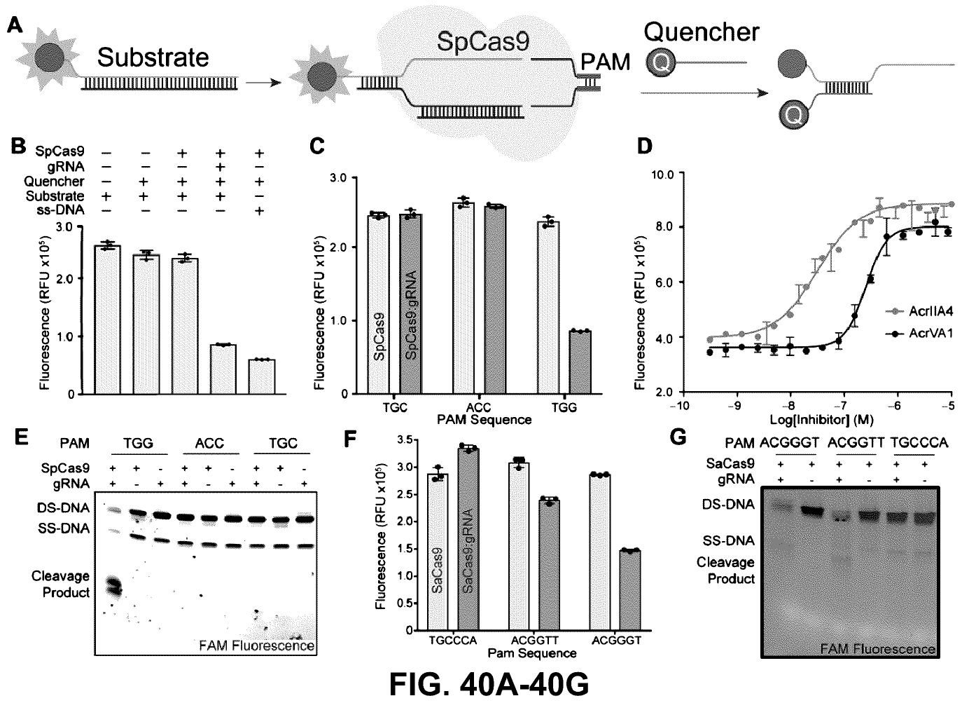

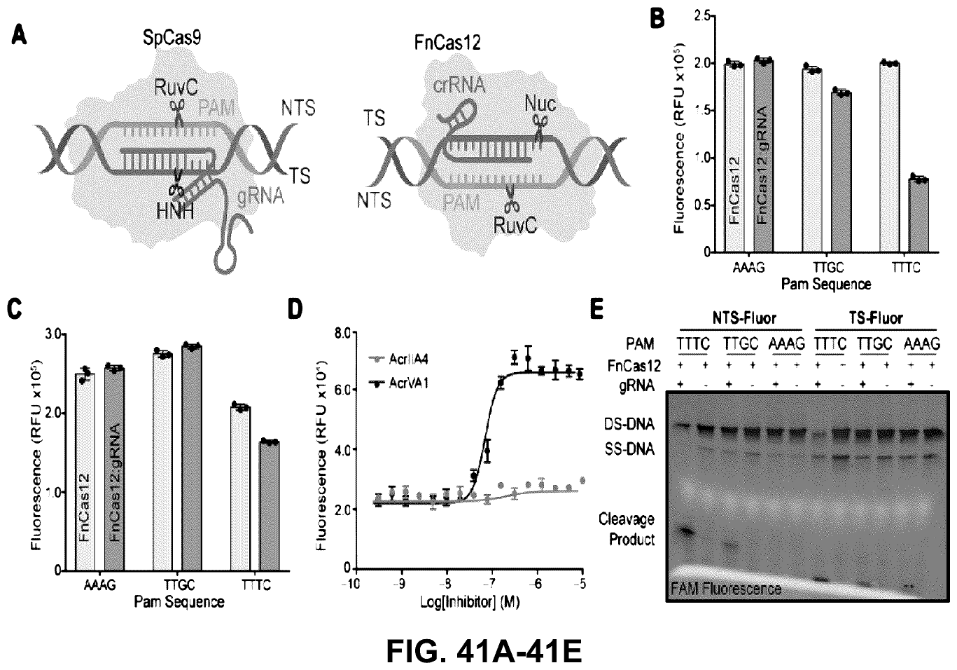

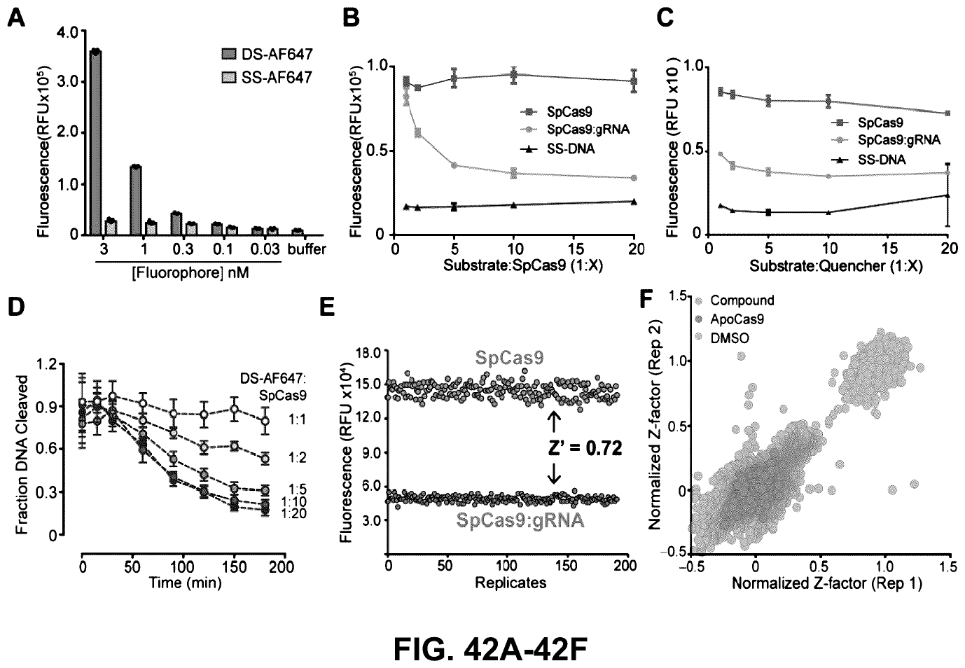

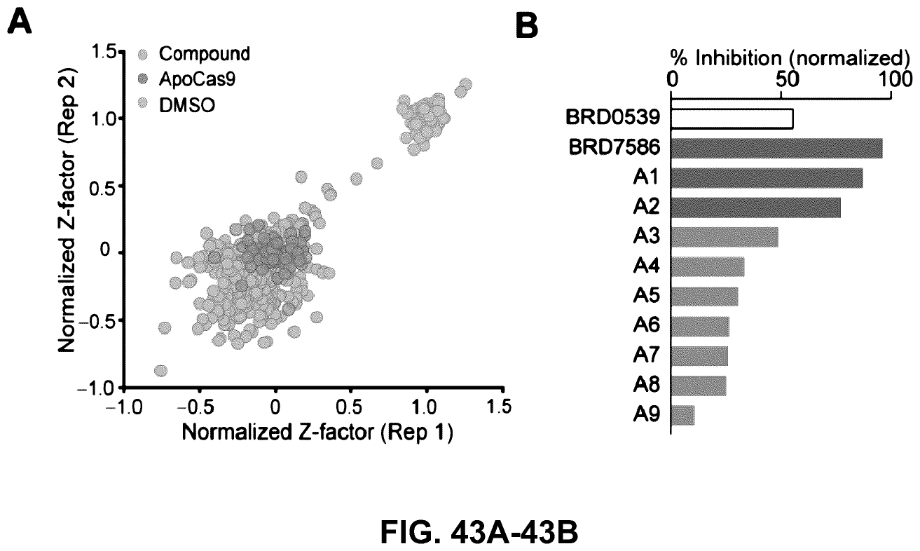

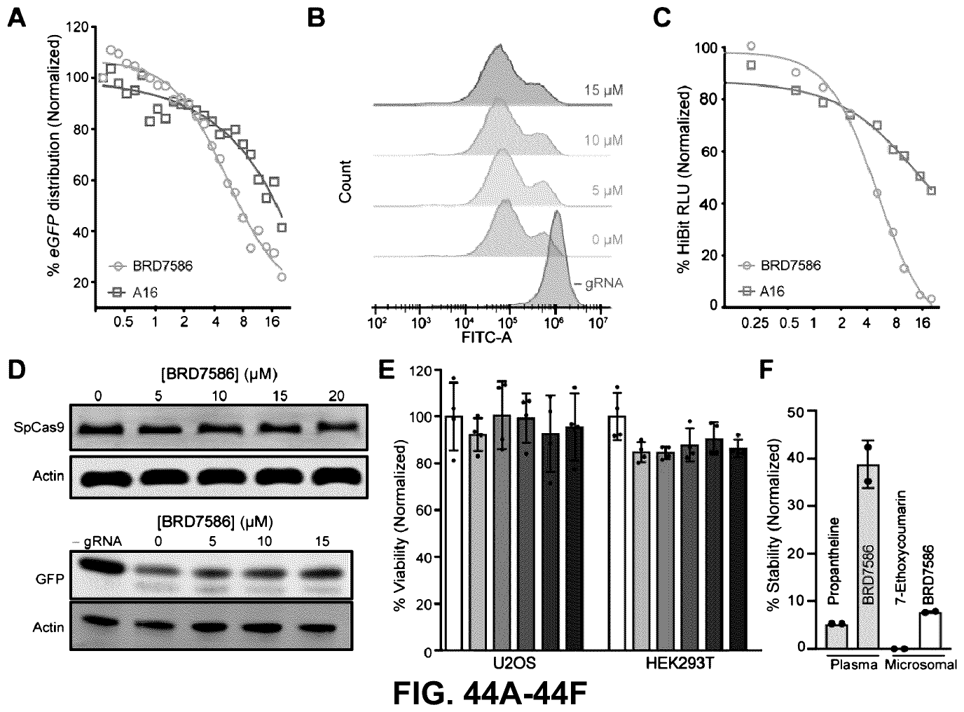

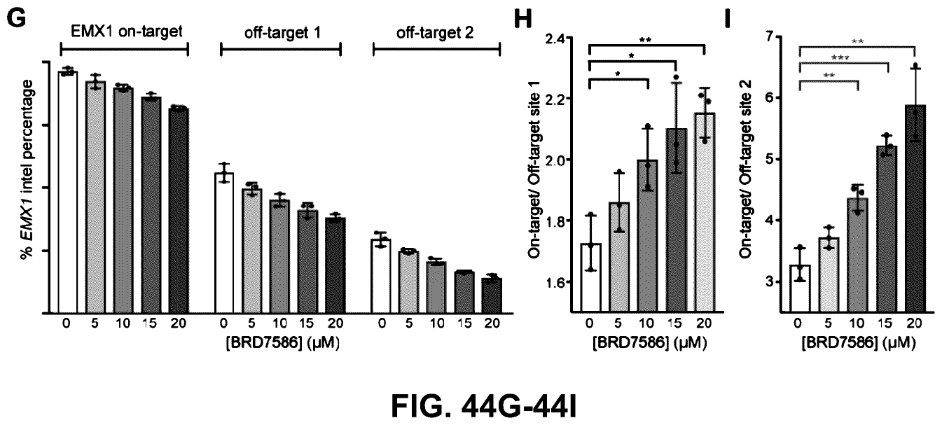

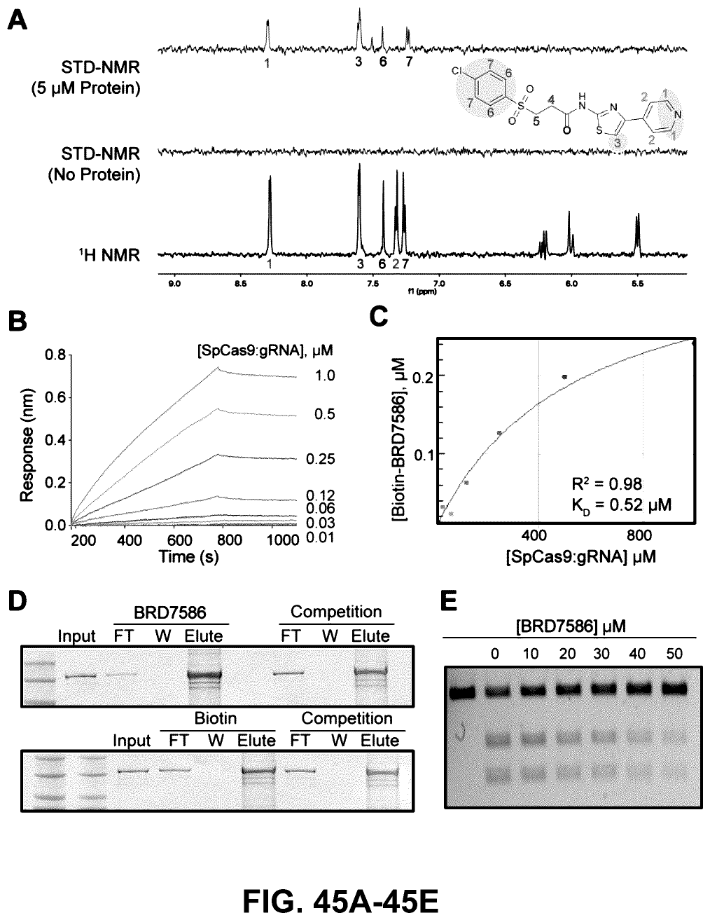

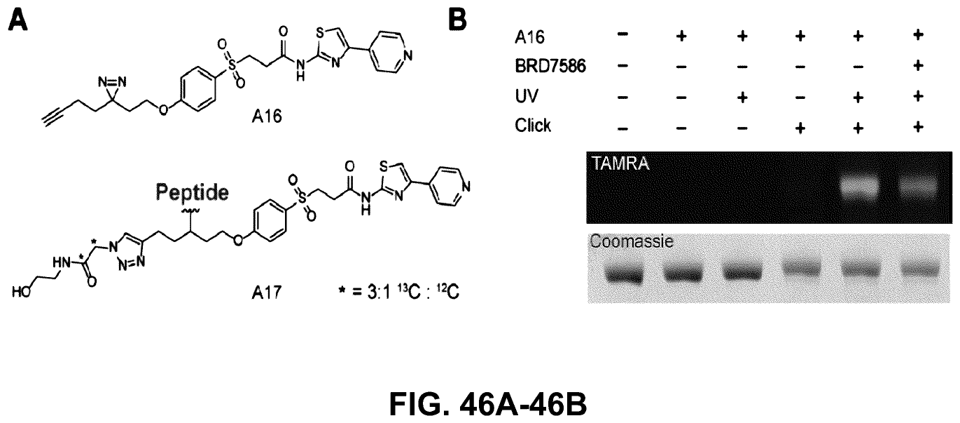

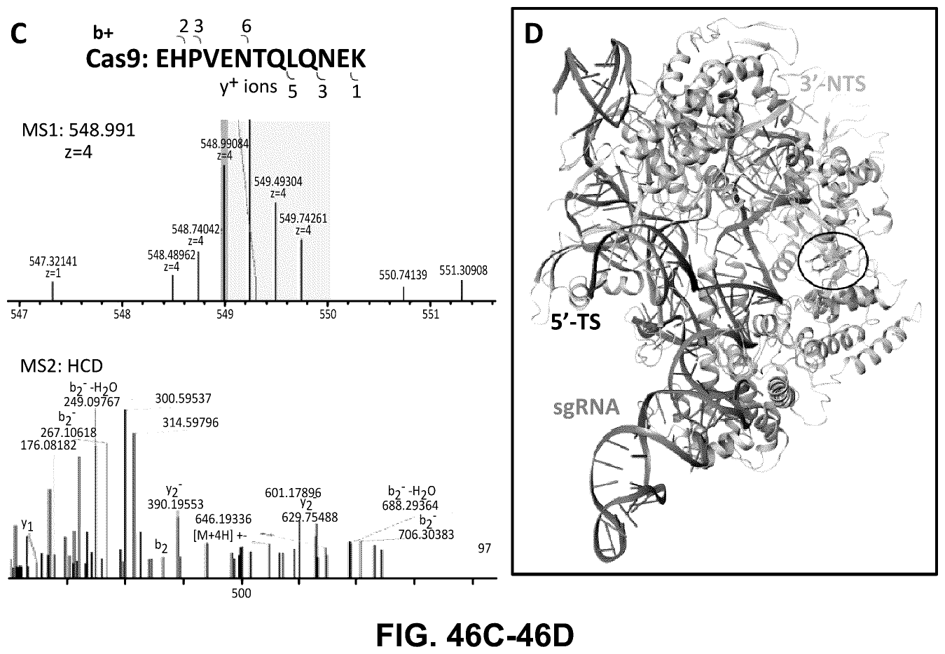

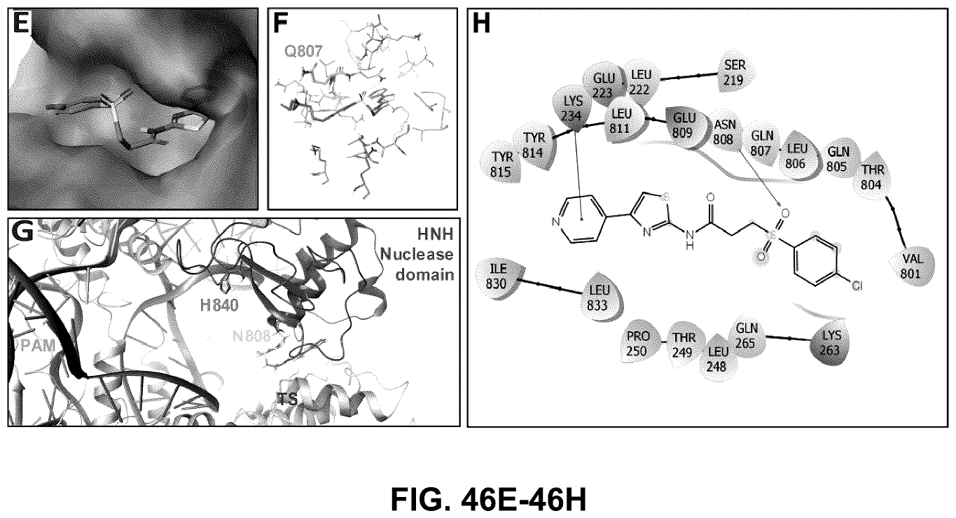

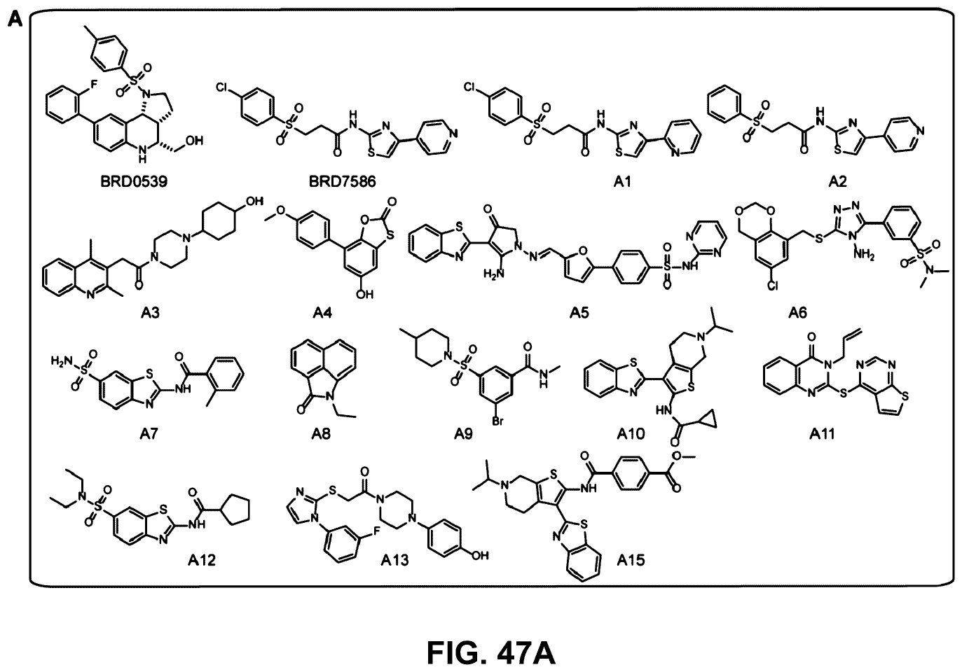

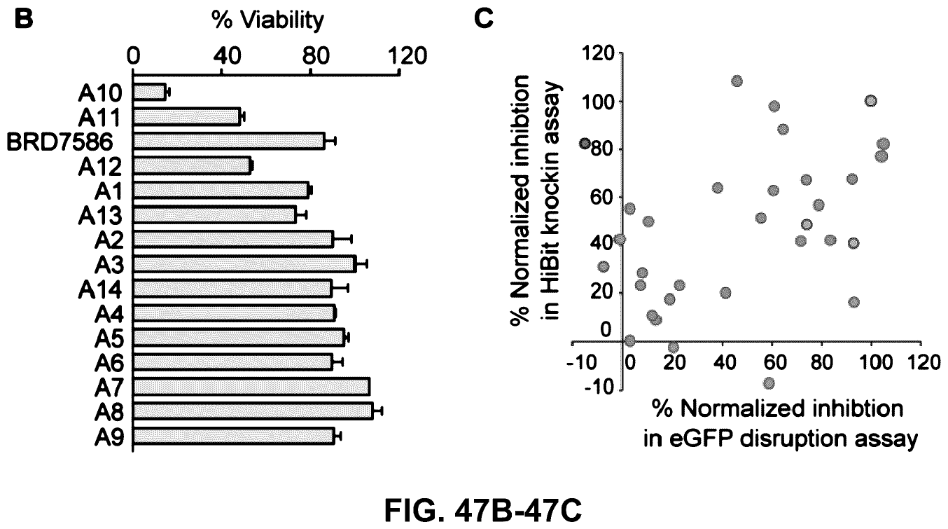

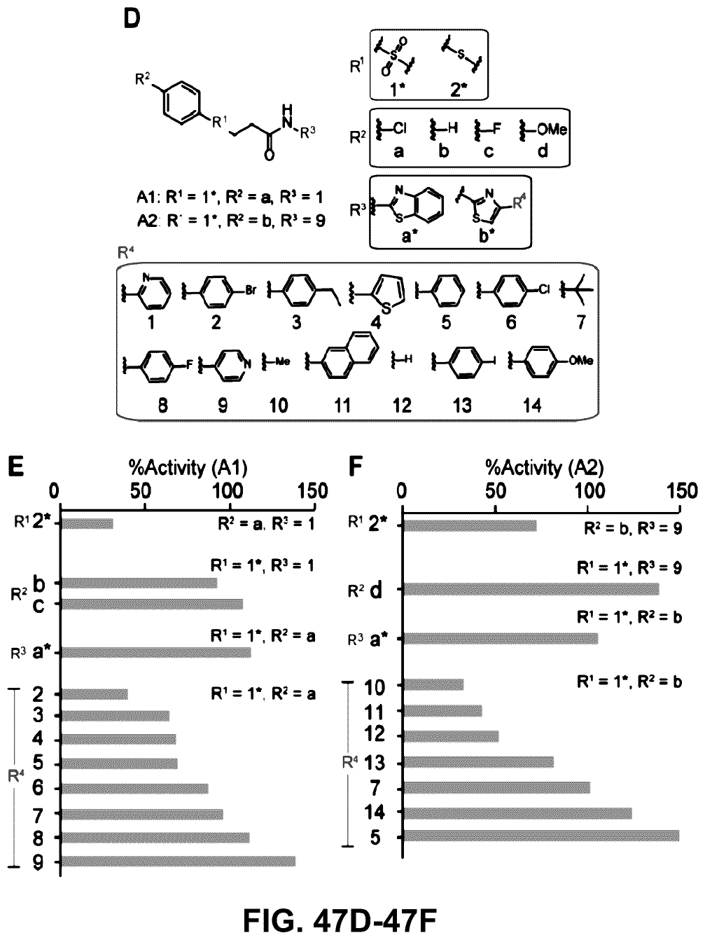

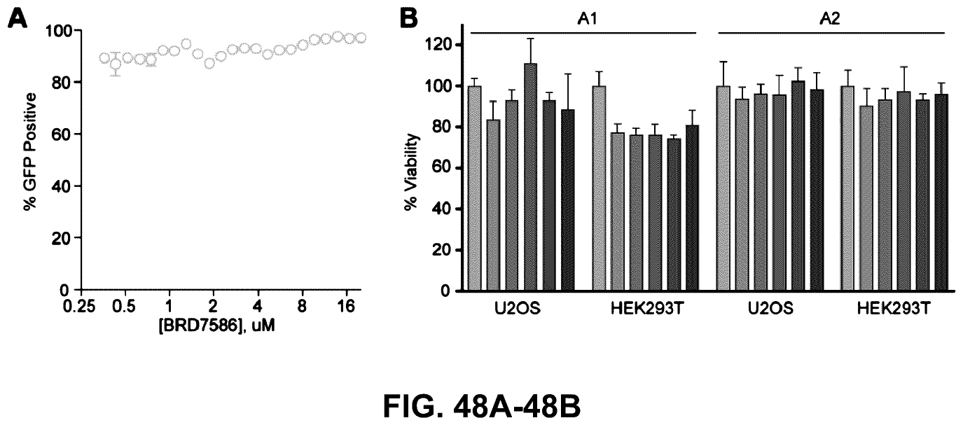

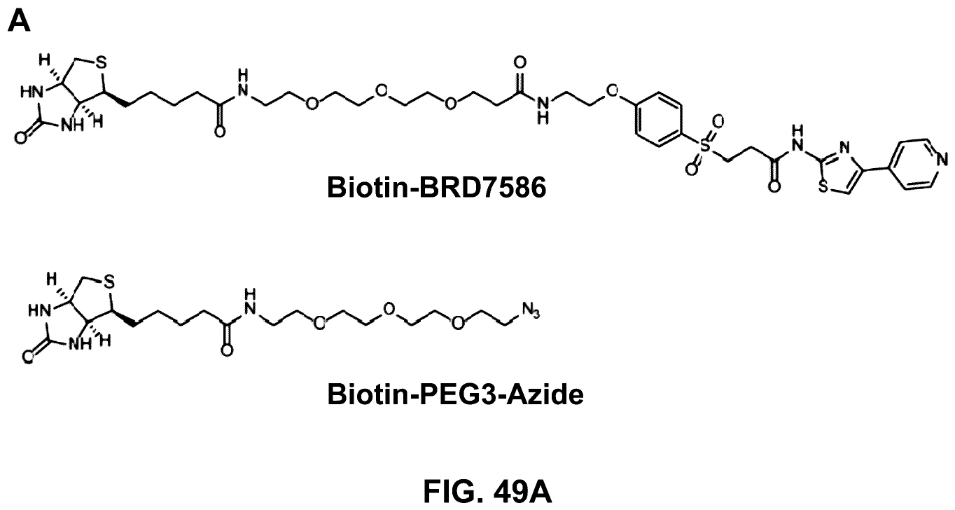

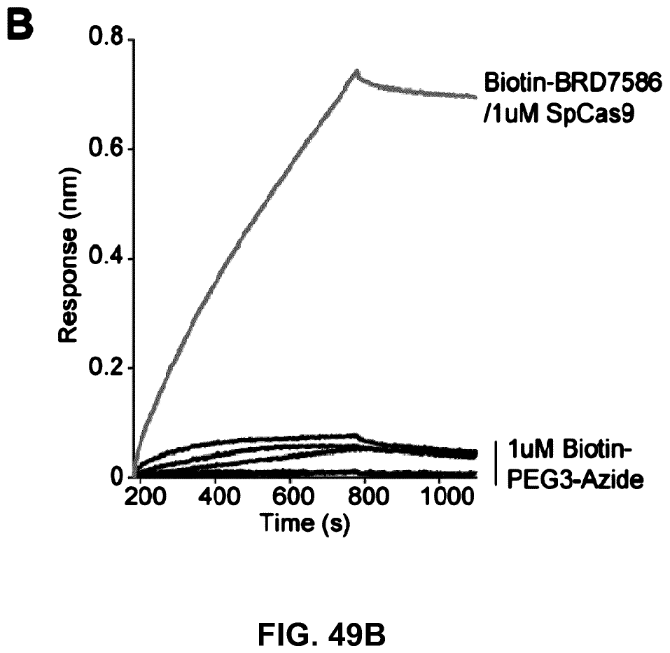

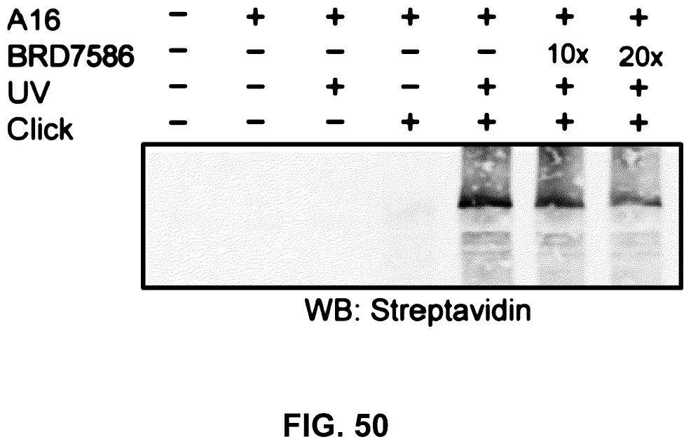

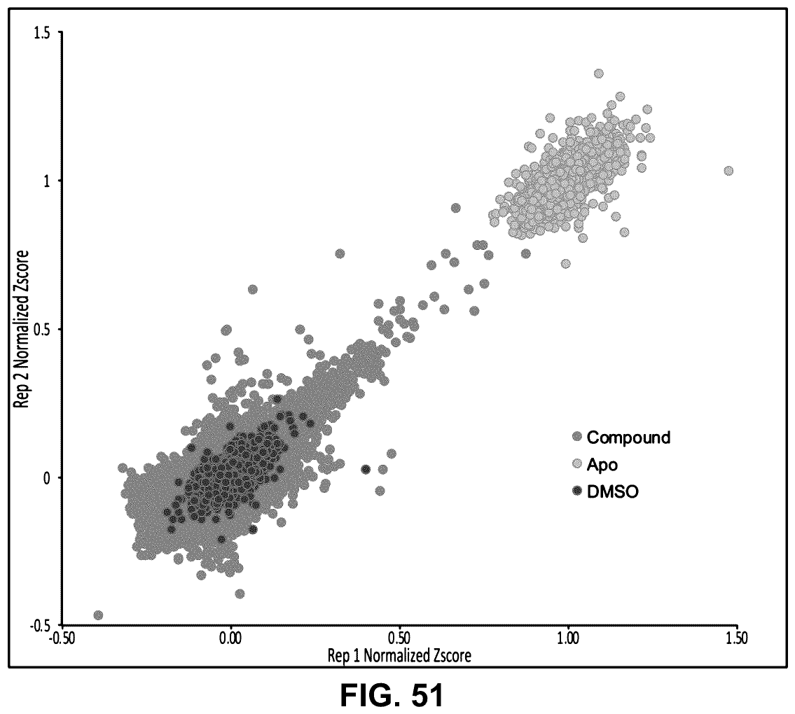

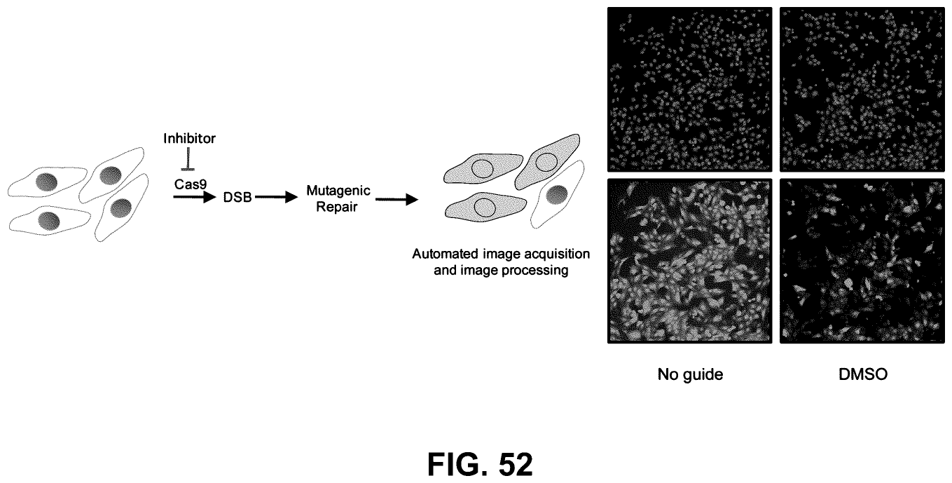

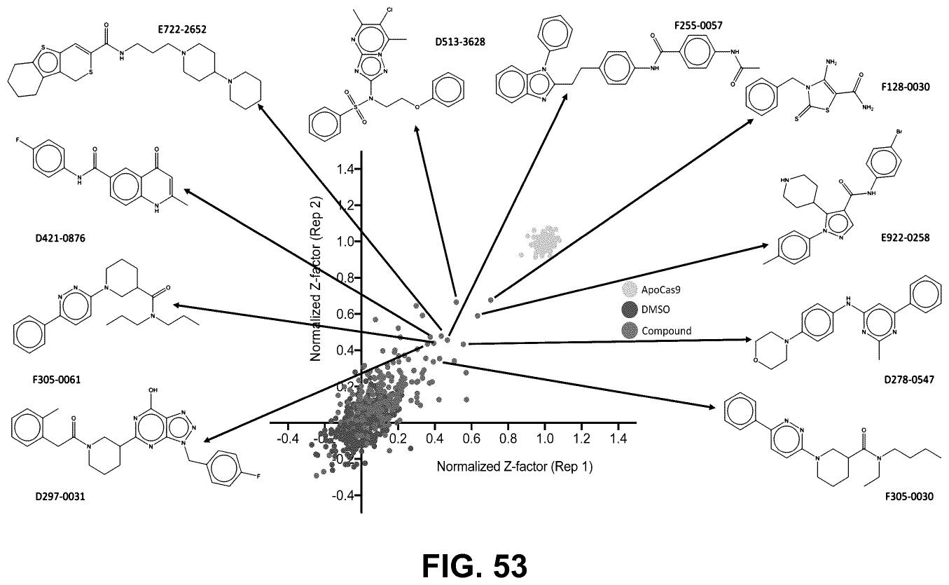

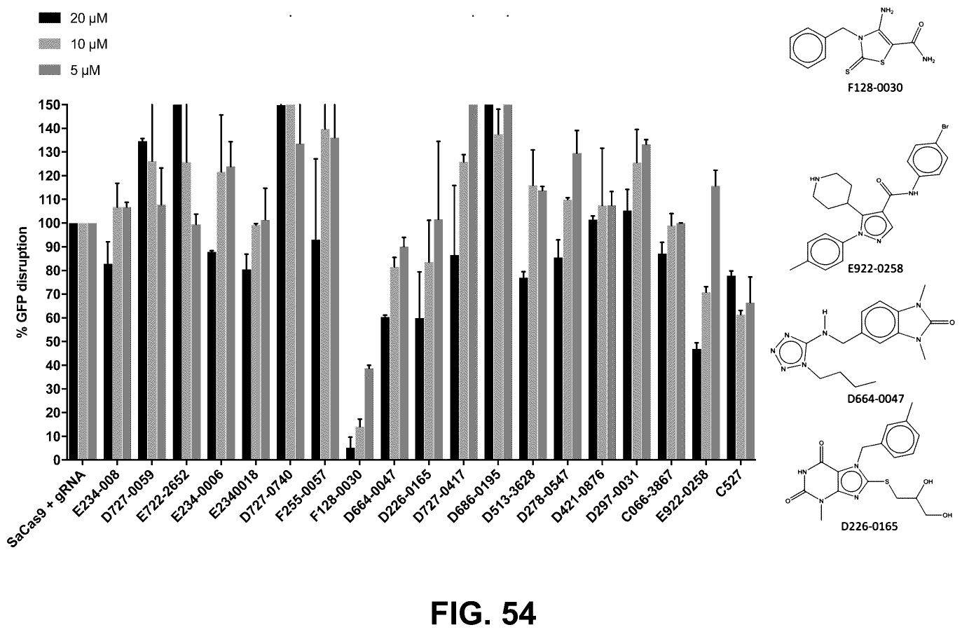

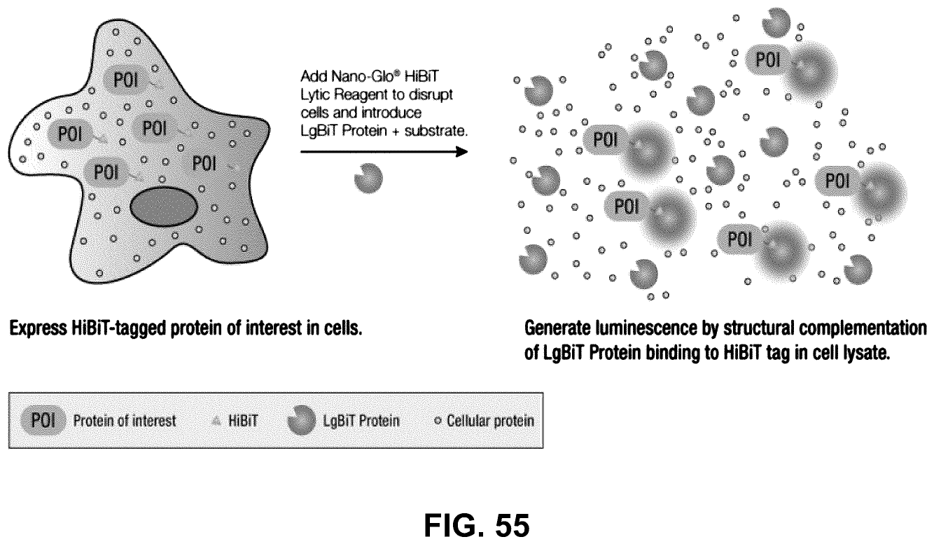

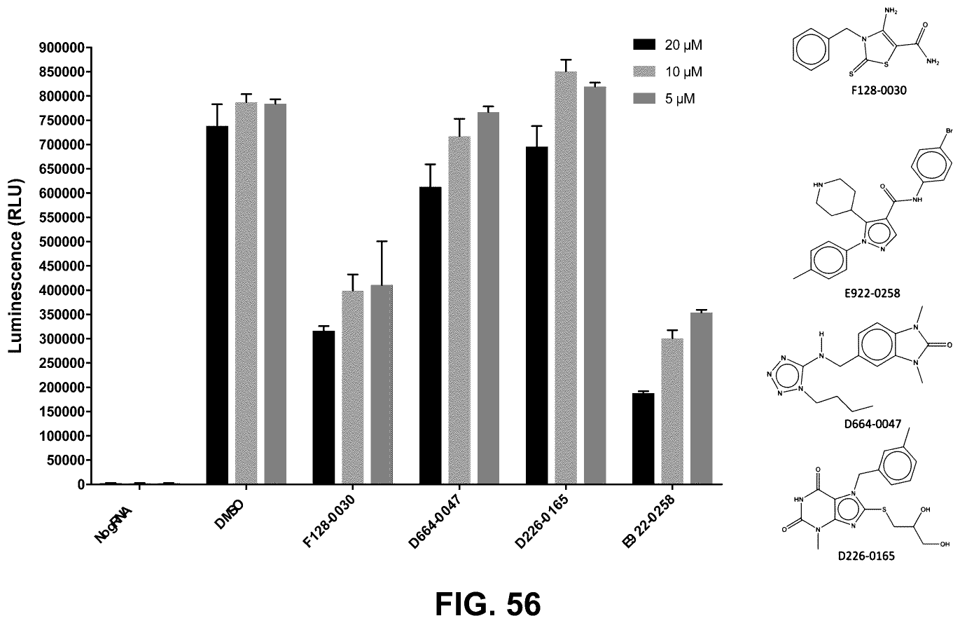

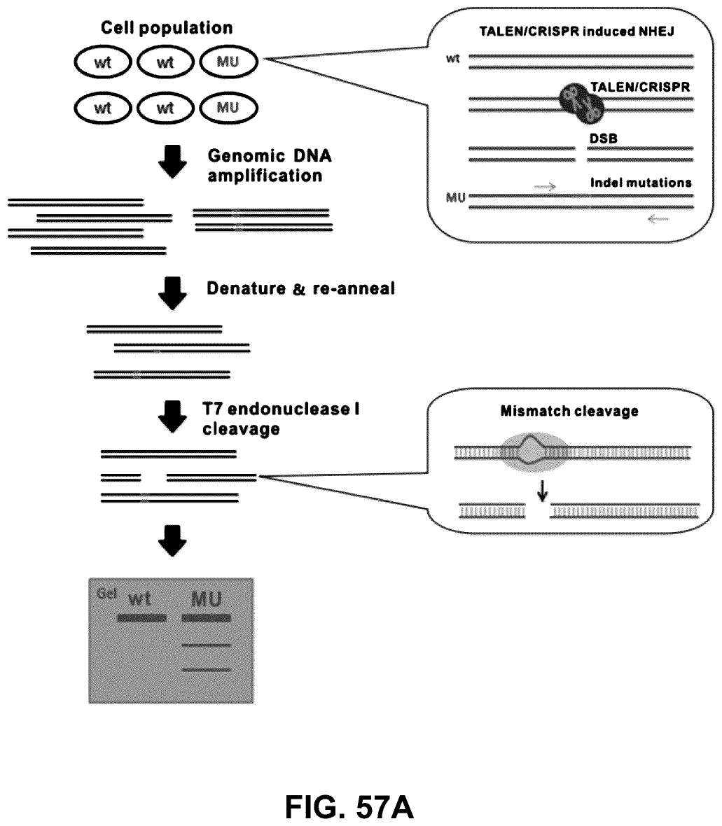

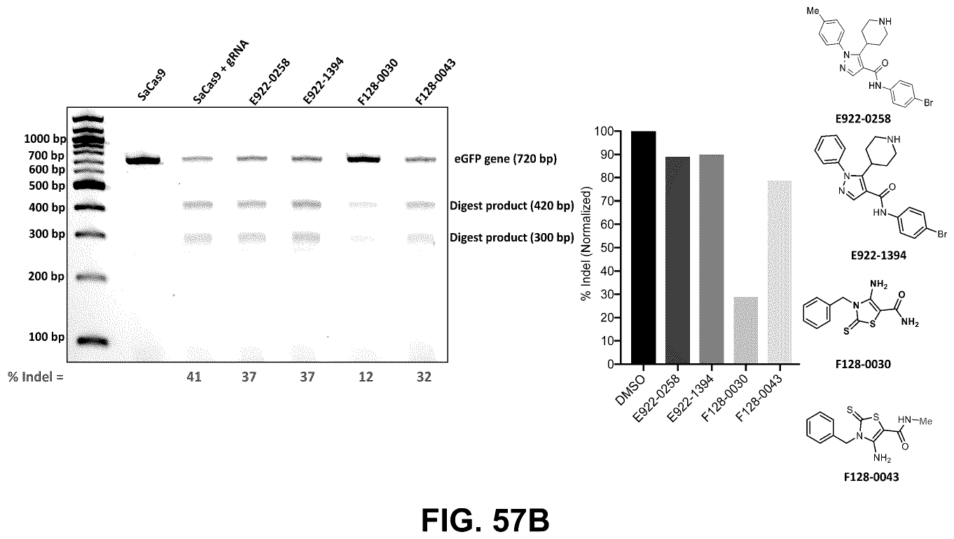

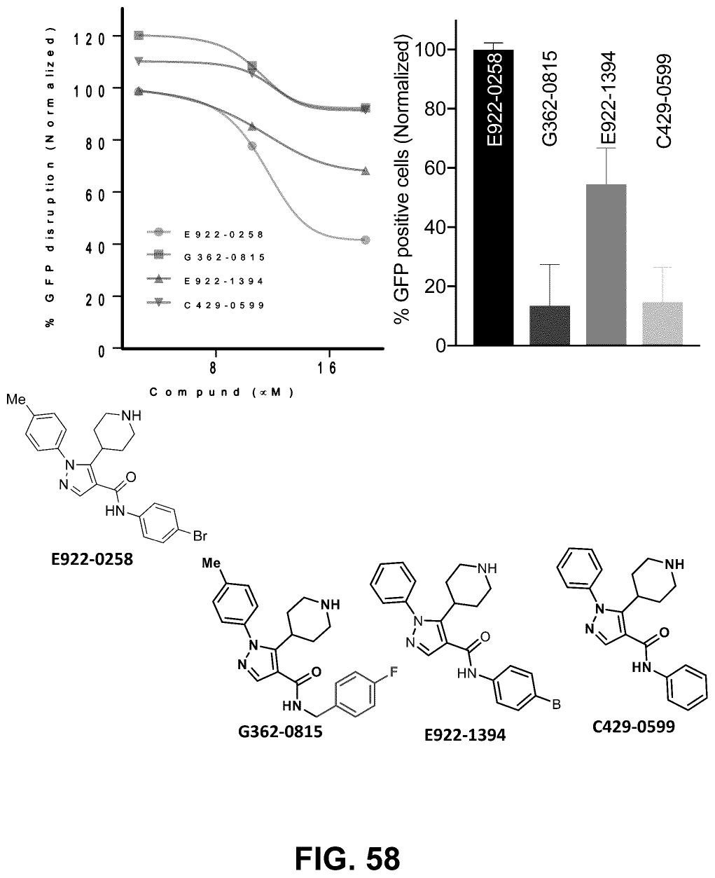

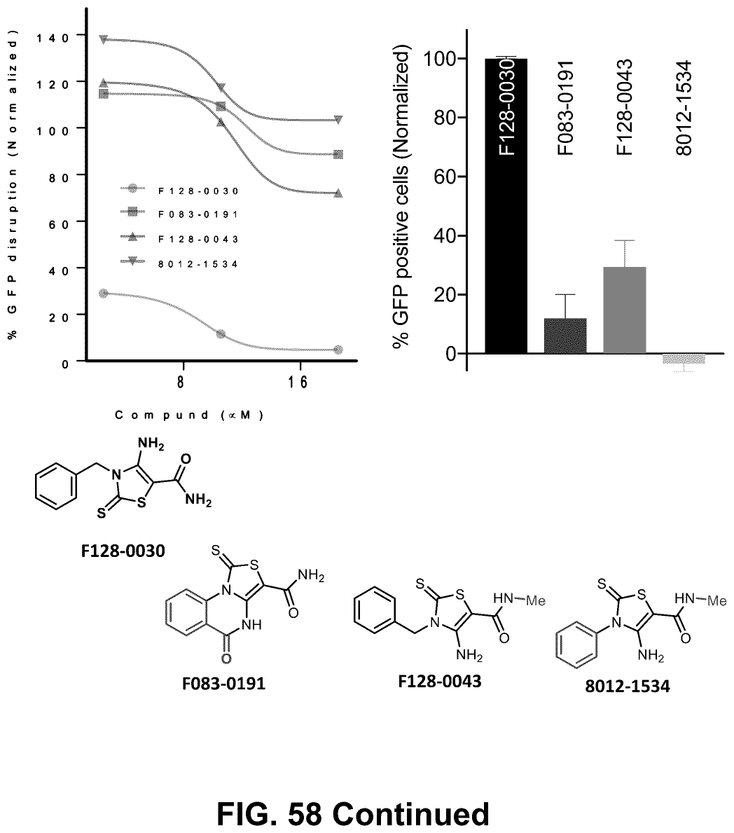

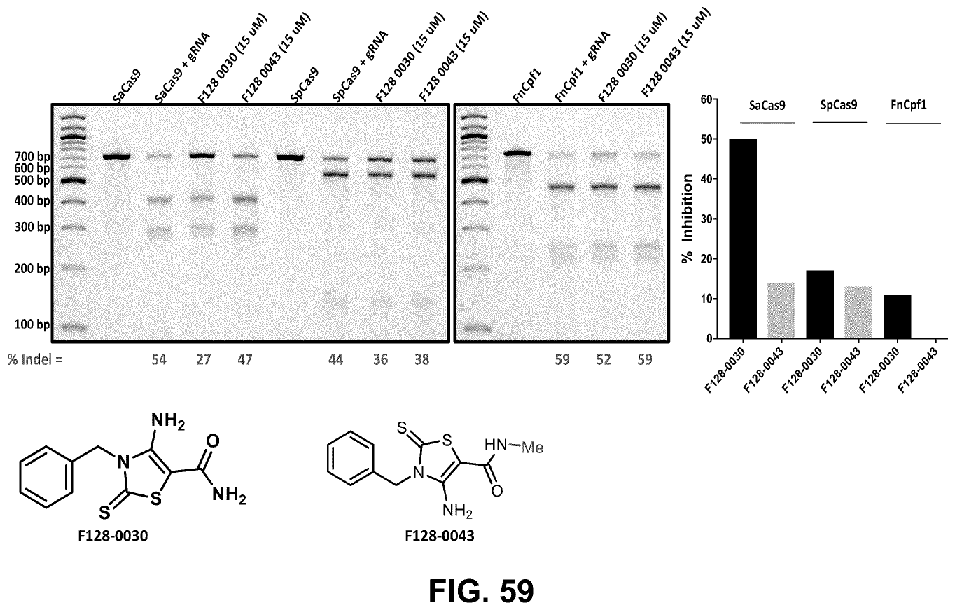

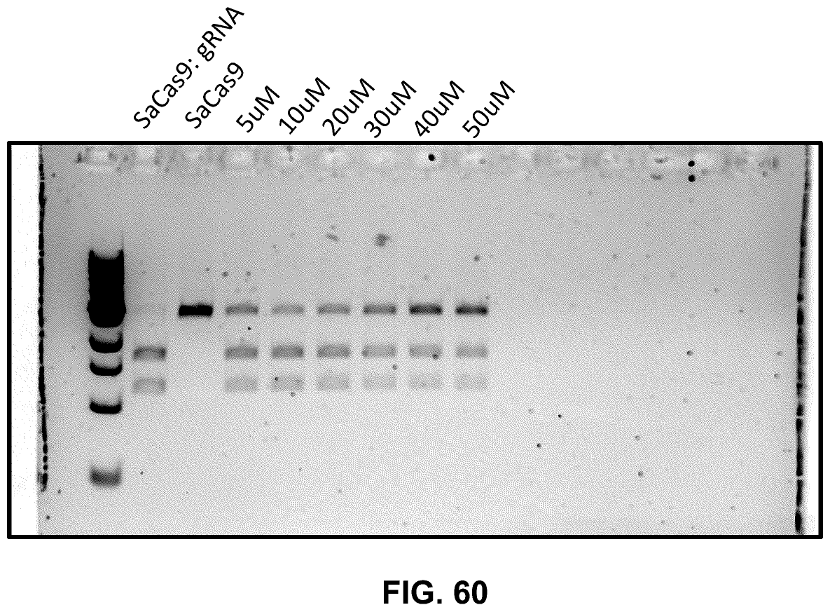

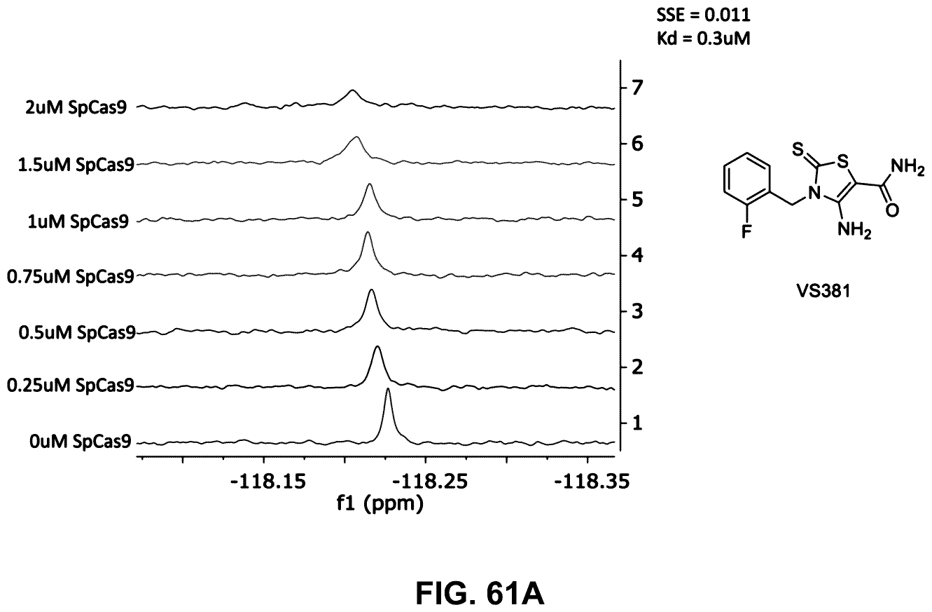

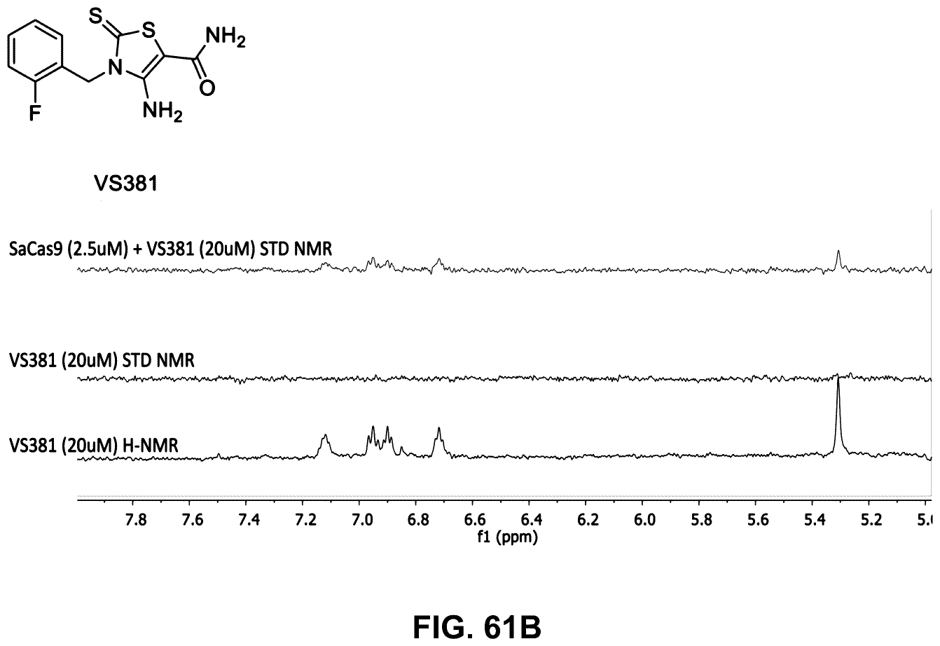

Structure Activity Relationship (SAR) of CD25 was evaluated (FIG. 32). Compounds are as defined below. SpCas9 inhibitor increased specificity for CD25 for on-target and off-target sites are shown FIG. 34. In particular embodiments, the Inhibitor can be according to the formula:

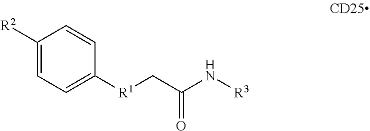

wherein R₁, R₂, R₃ and R₄ can be independently selected from Table 3C.

TABLE 3C
SpCas9 Inhibitors

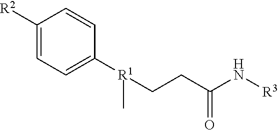

CD25:
R¹

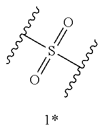 [SMILES: *S(*)(=O)=O]

R²

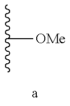 [SMILES: *OC]

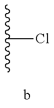 [SMILES: *Cl]

R³

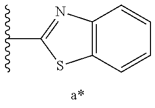 [SMILES: *c1nc2ccccc2s1]

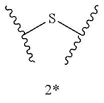 [SMILES: *S*]

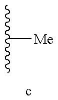

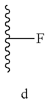 [SMILES: *F]

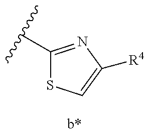

R⁴

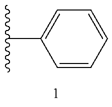 [SMILES: *c1ccccc1]

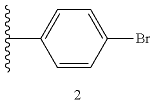 [SMILES: *c1ccc(Br)cc1]

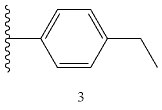 [SMILES: *c1ccc(CC)cc1]

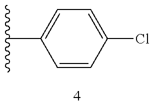 [SMILES: *c1ccc(Cl)cc1]

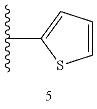 [SMILES: *c1cccs1]

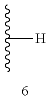 [SMILES: *[H]]

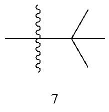 [SMILES: *C(C)(C)C]

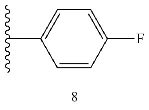 [SMILES: *c1ccc(F)cc1]

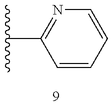 [SMILES: *c1ccccn1]

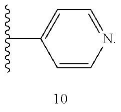 [SMILES: *c1ccncc1]

R¹ = 1*,
R² = b,
R³ = 10

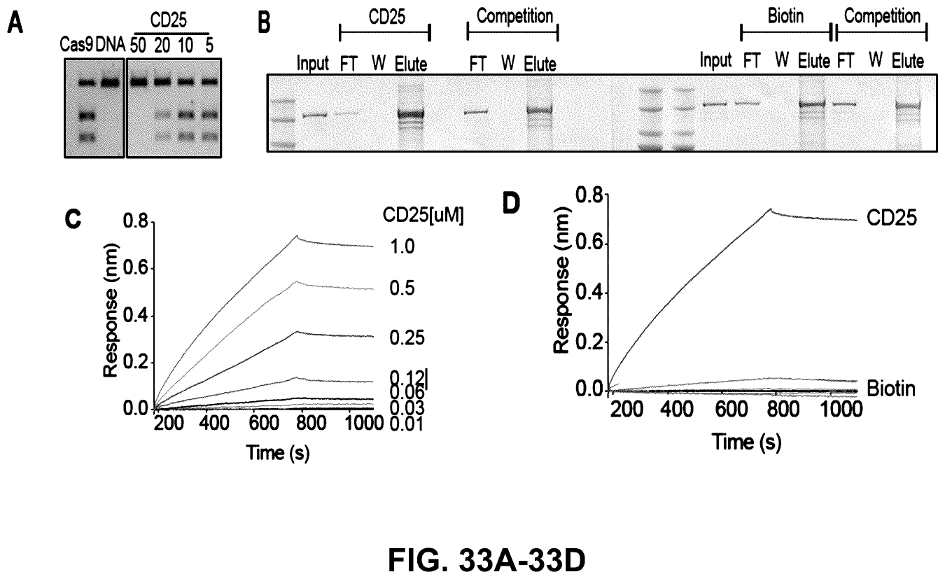

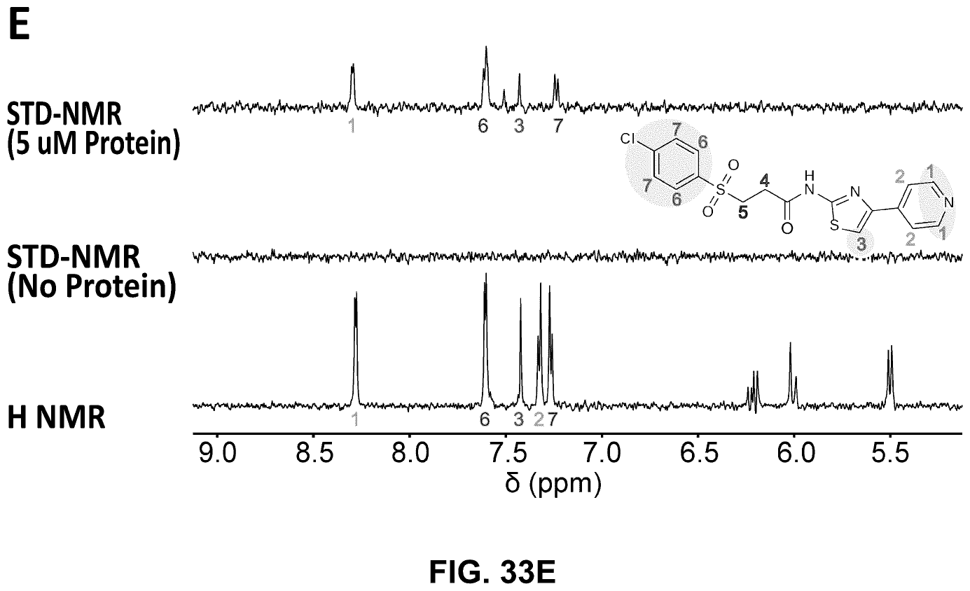

Additional variants to substituent can be explored based, at least in part, on the results of the NMR, shown in FIG. 33E identifying potential binding atoms of CD25 (also referred to herein as BRD7586) bound to SpCas9. In embodiments, preferred diazirine analogs are preferred. Based on the identified binding atoms, additional SAR can be undertaken to enhance specificity of the inhibitors, utilizing techniques as described elsewhere herein. In particular embodiments, the SpCas 9 inhibitor can be selected according to the following formula according to A1 or A2:

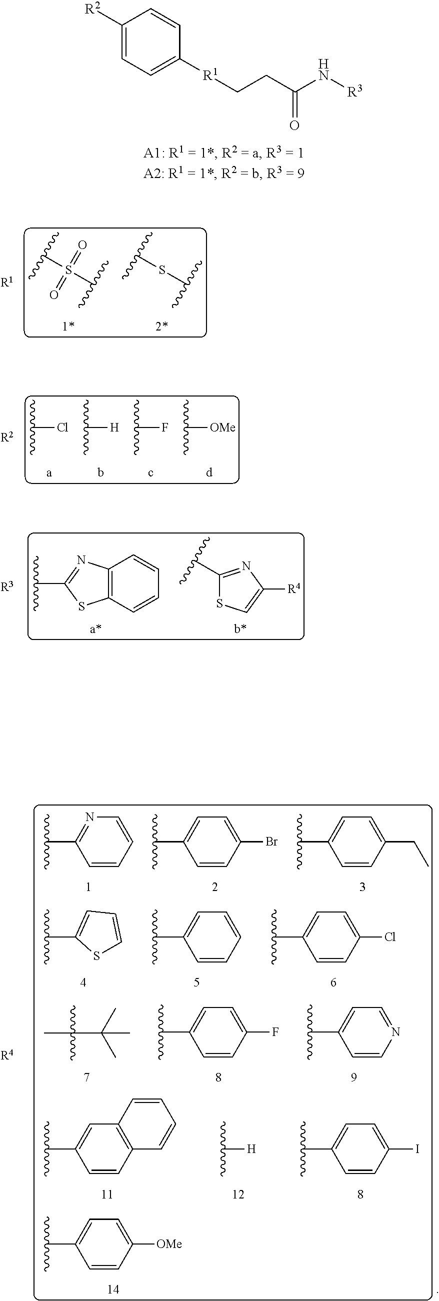

In certain embodiments, the molecule is according to

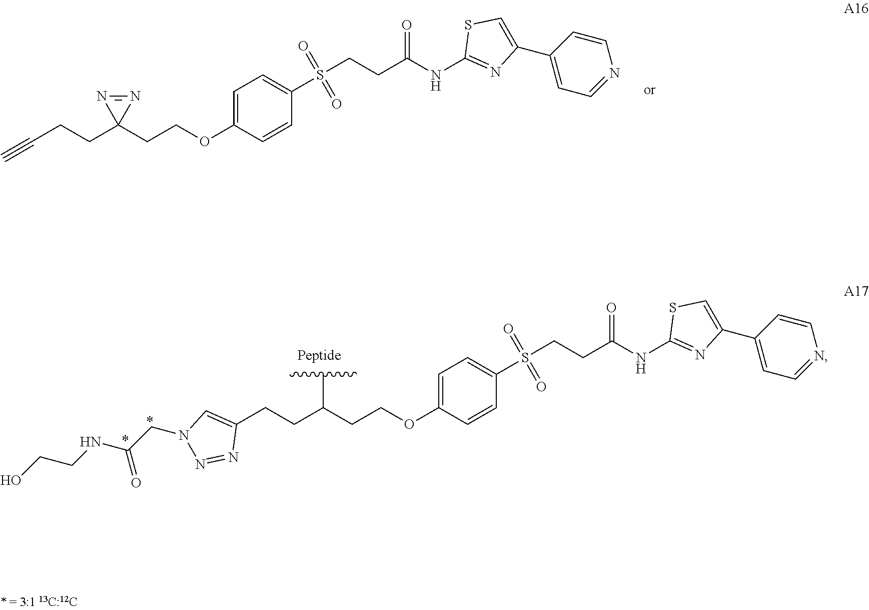 [SMILES: C#CCCC1(CCOc2ccc(S(=O)(=O)CCC(=O)Nc3nc(-c4ccncc4)cs3)cc2)N=N1 ; *C(CCOc1ccc(S(=O)(=O)CCC(=O)Nc2nc(-c3ccncc3)cs2)cc1)CCc1cn(CC(=O)NCCO)nn1]

or derivatives thereof.

TABLE 3D
Compound libraries from ICCB-L screened and compound number from each library
identified as having inhibition against SpCas9 cleavage activity.

| | | | No. of | | No. of |
|---|---|---|---|---|---|
| | | | positive | No. of | positive |
| Compound | | No. of | compounds in | cherrypicked | compounds in |
| group | Library name | compounds | primary screen | compounds* | secondary screen |
| Commercial | ChemDiv6 | 44,000 | 93 (0.21% hit | 83 | 0 |
| compounds | | | rate) | | |
| | ChemDiv7 | 49,128 | 382 (0.77% hit | 374 | 8 |
| | | | rate) | | |
| | Enamine 2 | 26,929 | 90 (0.33% hit | 79 | 8 |
| | | | rate) | | |
| Known | NIH Clinical | 450 | 4 (0.89% hit | 4 | 0 |
| bioactive | Collection 1 - 2014 | | rate) | | |
| compounds | Selleck Bioactive | 1,902 | 10 (0.53% hit | 7 | 0 |
| | Compound Library | | rate) | | |

*PAINS flagged compounds removed from the hit list.

Anti-CRISPR proteins may be utilized in applications as well and may be used in conjunction with the small molecule inhibitors disclosed herein. See, e.g., Etzinger et al., doi:10.1101/854950.

### SaCas9 Inhibitors

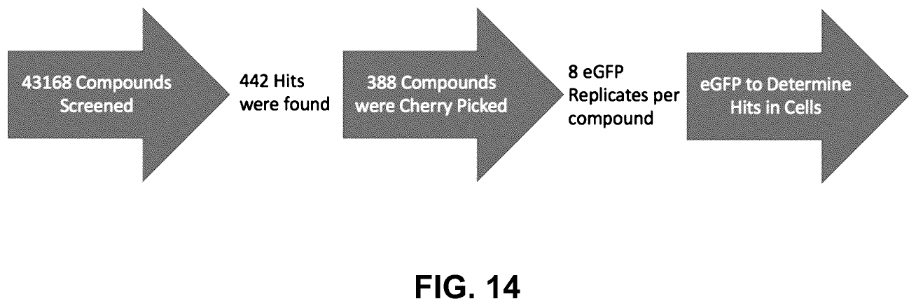

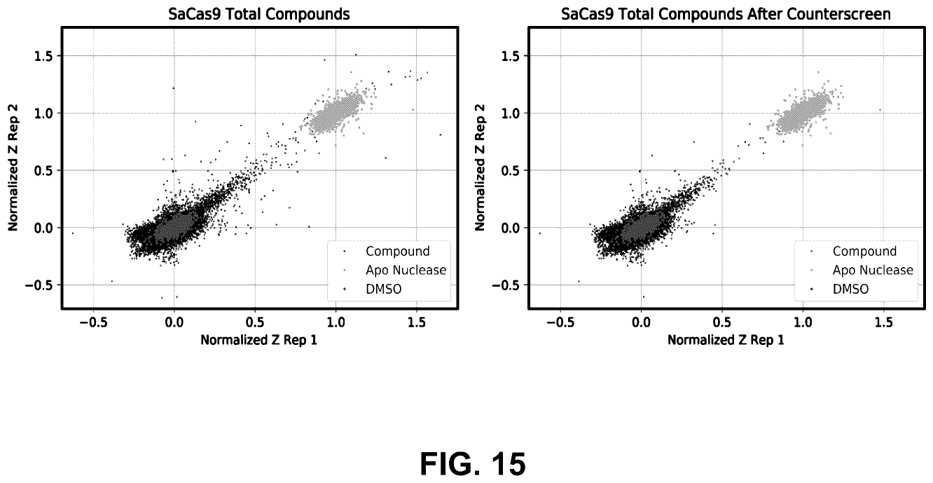

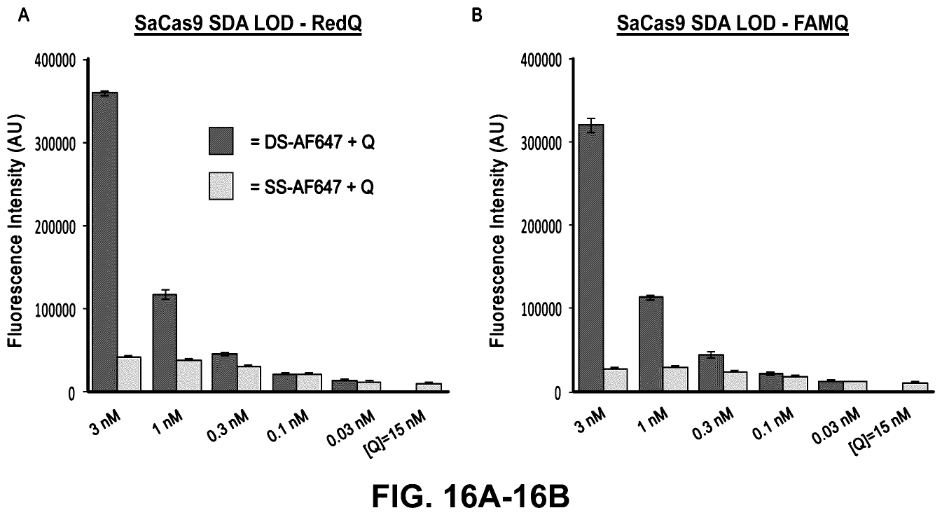

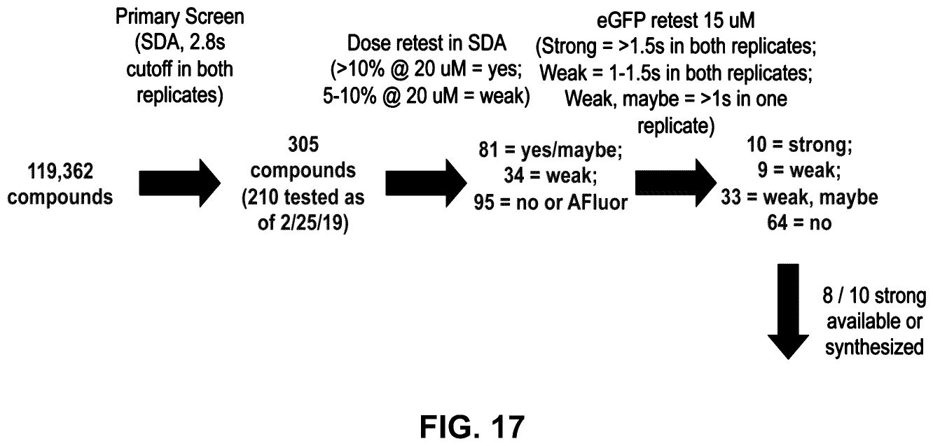

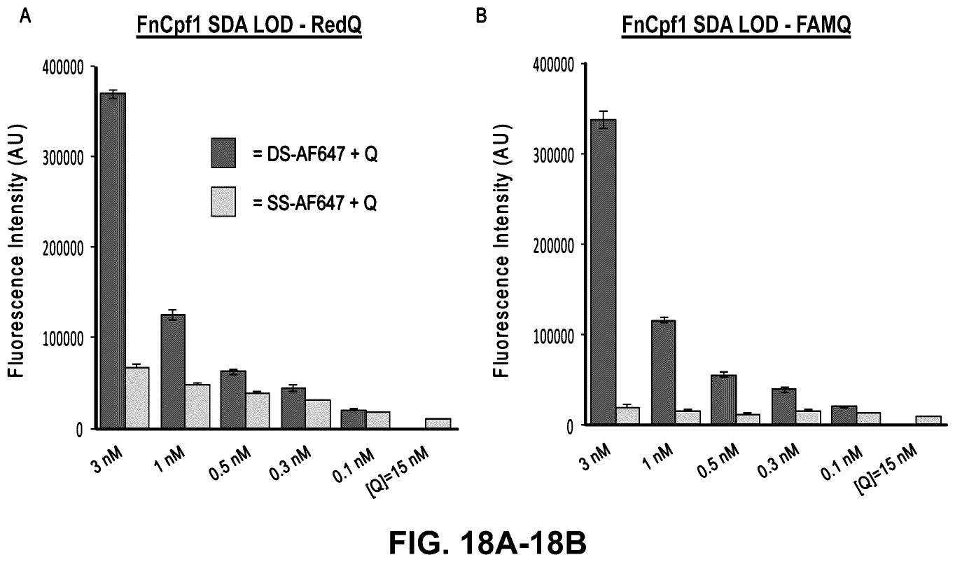

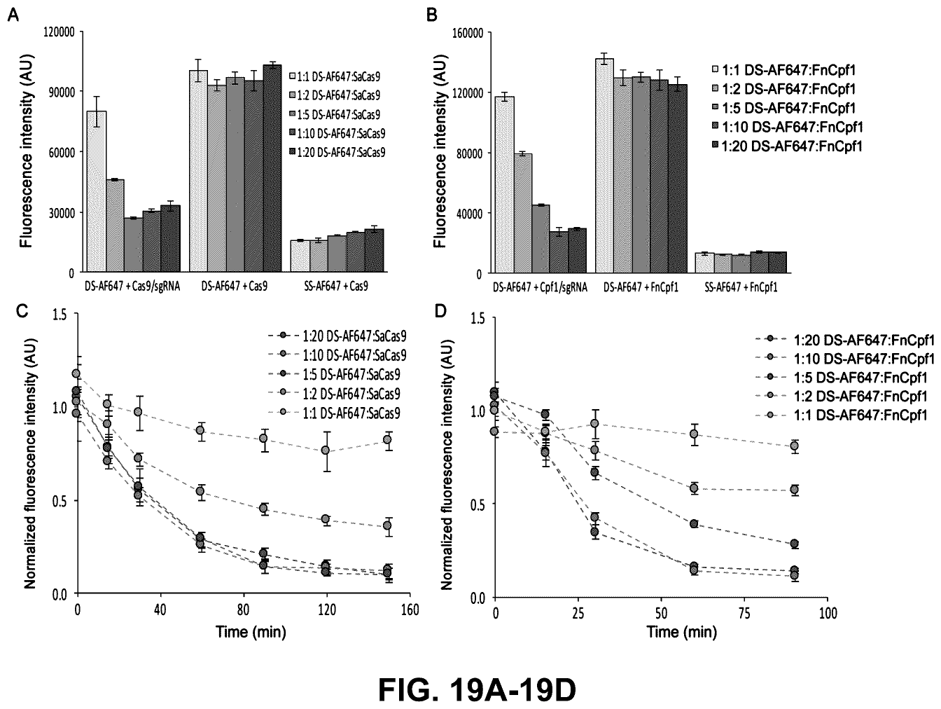

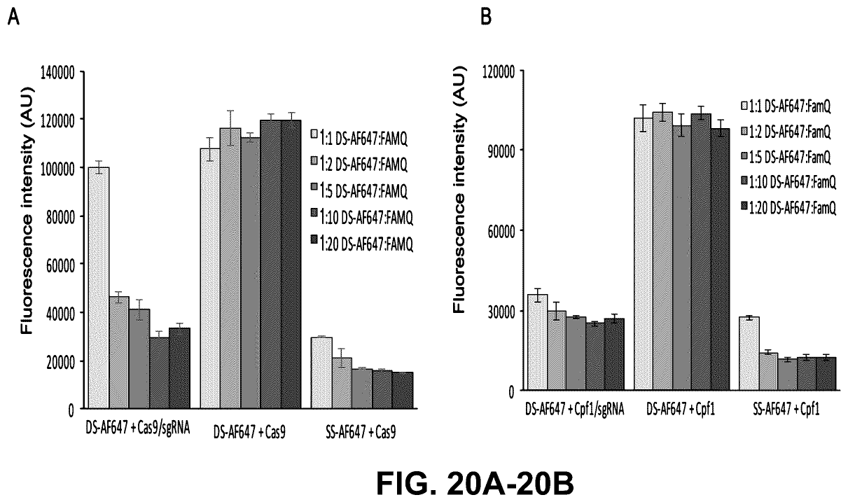

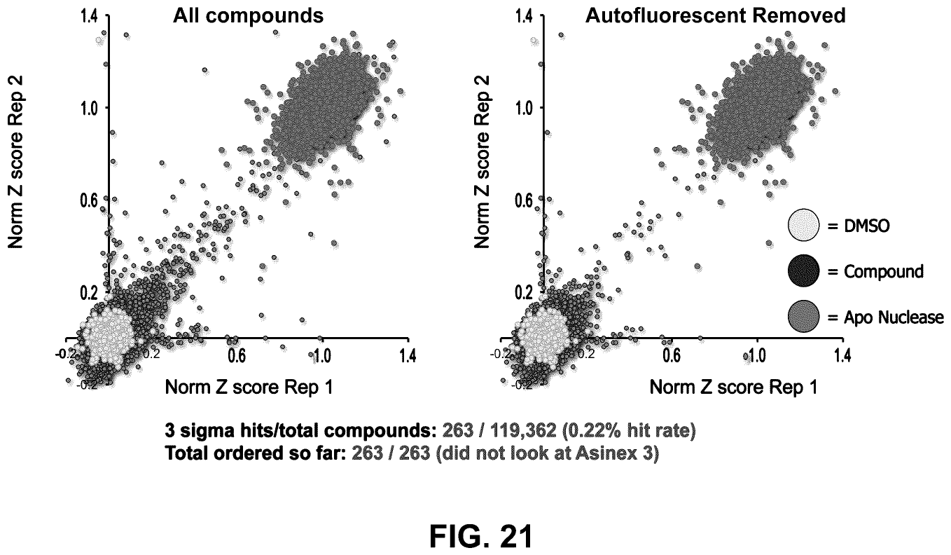

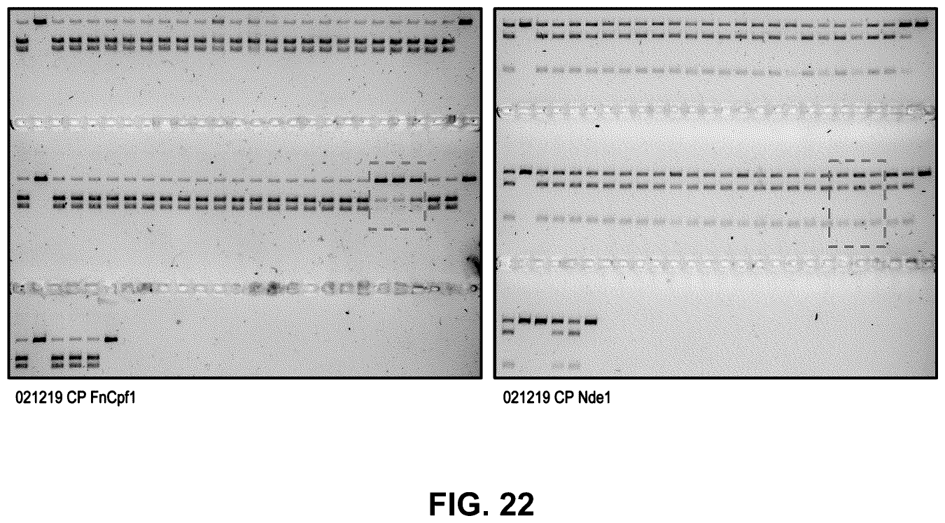

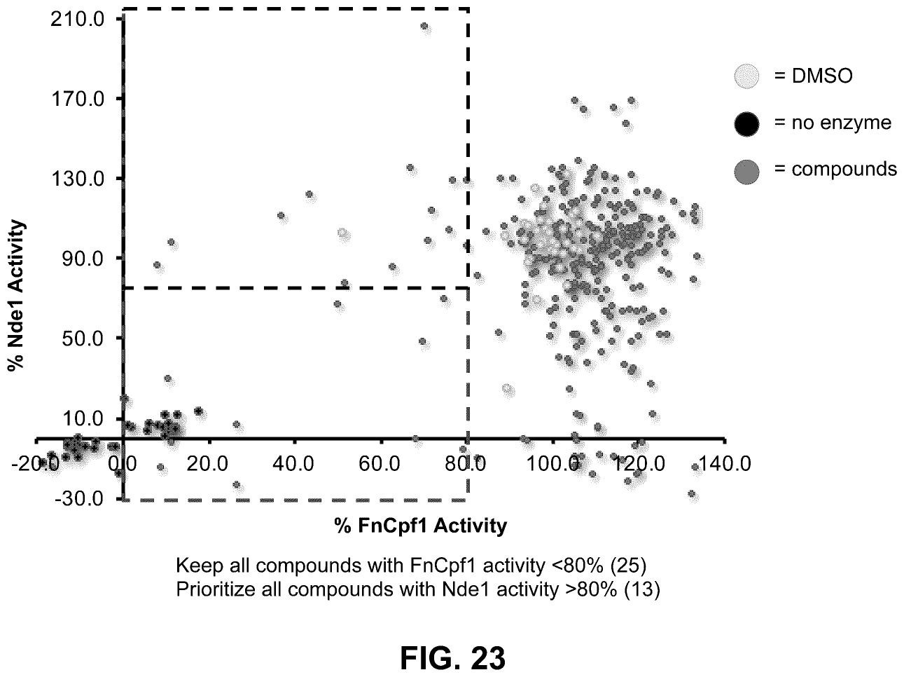

An overview of the screening for SaCas9 inhibitors is depicted in FIG. 14. A total of 43,168 Compounds were screened from the ICCB Libraries. Libraries screened for the initial compounds include ChemDiv 7 (34,848 cpds), Enamine 1 (5,280 cpds), ChemDiv1 (1,760 cpds), NIH Clinical Collection (450 cpds), Biomol 4—FDA Approved Library (640 cpds). A hit is determined from the Zscore, with 3-5σ being a weak hit, >5σ being a strong hit. The results of the hits include 193 Weak Hits and 249 Strong Hits. The NIH Clinical Collection 1-2014 was screened for 450 of 450 compounds, with 7 hits—1.5%. The Biomol 4—FDA Approved Library was screened for 640 of 640 compounds, with 8 hits—1.2%. ChemDiv 1 was screened for 1760 of 28,864 compounds, with 19 hits—1.07%. ChemDiv 7 was screened for 34,848 of 49,128 compounds, with 364 hits—1.04%. Enamine 1 was screened for 5,280 of 6,004 compounds, with 44 hits—0.83%. Libraries are commercially available, with reference numbers utilized for compounds identifying the compound structures used.

Further small-molecule screening and hit identification included primary screening of 95,241 compounds, with 4621 removed by counterscreen, with a total of 1063 hits (1.1%). Screening included ChemDiv1, ChemDivTargeted Diversity, Enamine 1, Enamine 2, NIH Clinical Collections, and Biomol 4.

All hit compounds were moved forward into the secondary and tertiary screens, in which they were tested in cell-based assays. An eGFP assay is used to determine hits in cells as the secondary screen (eGFP assay).

In Table 4A below, the favorable strong hits for SaCas9 inhibitors screened thus far are provided, and in Table 4B weak hits are provided. Table 4C provides a total hit compounds that have been identified from the total of about 90,000 compounds in the primary screens.

TABLE 4A
SaCas9 Strong Inhibitor Hits

| | | Com- | |
|---|---|---|---|
| Com- | Com- | pound | |
| pound | pound | Vendor | |
| Lib | Vendor | ID | Compound SMILE |
| ChemDiv Targeted Diversity Library | Chem Div | D727- 0394 | 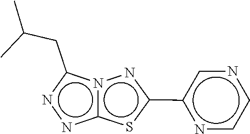 |
| | | | n12c(nnc1CC(C)C)sc(c3cnccn3)n2 |
| ChemDiv Targeted Diversity Library | Chem Div | C301- 5391 | 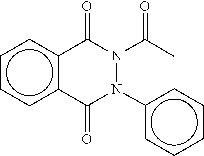 |
| | | | N1(C(C)═O)C═O)c2c(C(═O)N1c3ccccc3)cccc2 |
| ChemDiv Targeted Diversity Library | Chem Div | C200- 7168 | 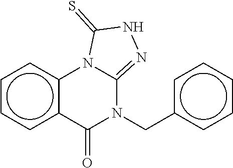 |
| | | | C1(═NNC2═S)N2c(cccc3)c3C(═O)N1Cc4ccccc4 |
| ChemDiv Targeted Diversity Library | Chem Div | D727- 0915 | 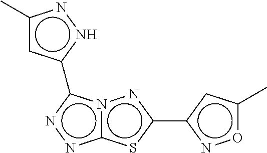 |
| | | | n12c(nnc1c3cc(n[nH]3)C)sc(c4cc(on4)C)n2 |
| ChemDiv Targeted Diversity Library | Chem Div | D727- 0182 | 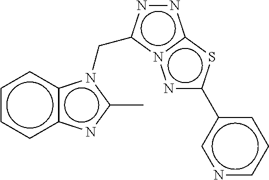 |
| | | | n12c(nnc1Cn(c3c(n4)cccc3)c4C)sc(c(cccn5)c5)n2 |
| ChemDiv Targeted Diversity Library | Chem Div | E218- 0296 | 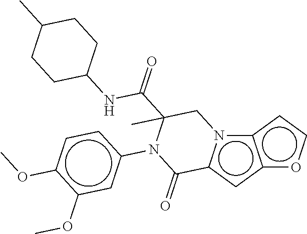 |
| | | | C(C)(C1)(C(═O)NC2CCC(CC2)C)N(c3ccc(c(OC)c3)OC)C(c4n1c5c(c4) [symbol] |
| ChemDiv Targeted Diversity Library | Chem Div | C066- 5401 | 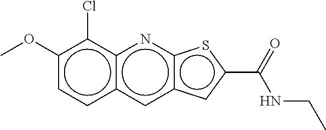 |
| | | | n1c2c(ccc(OC)c2Cl)cc(c1s3)cc3C(NCC)═O |
| ChemDiv Targeted Diversity Library | Chem Div | D727- 0417 | 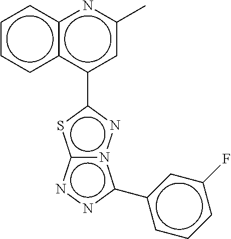 |
| | | | n12c(nnnc1c3cccc(F)c3)sc(c(cc4C)c5c(n4)cccc5)n2 |
| ChemDiv Targeted Diversity Library | Chem Div | C200- 9572 | 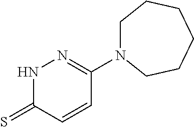 [SMILES: S=c1ccc(N2CCCCCC2)n[nH]1] |
| | | | N1═C(C═CC(═S)N1)N2CCCCCC2 |
| ChemDiv Targeted Diversity Library | Chem Div | D727- 0404 | 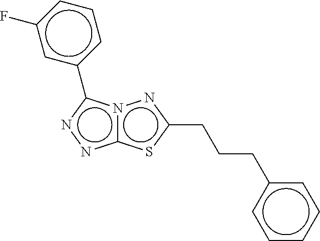 |
| | | | n1(nc(s2)CCCc3ccccc3)c2nnc1c4ccccc(F)c4 |
| ChemDiv Targeted Diversity Library | Chem Div | C200- 7260 | 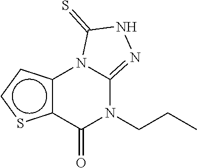 |
| | | | C1(═NNC2═S)N2c(c3C(═O)N1CCC)ccs3 |
| ChemDiv Targeted Diversity Library | Chem Div | D727- 0892 | 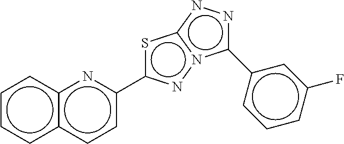 |
| | | | n12c(nnc1c3cccc(F)c3)sc(c4ccc(c5n4)cccc5)n2 |
| ChemDiv Targeted Diversity Library | Chem Div | E198- 0044 | 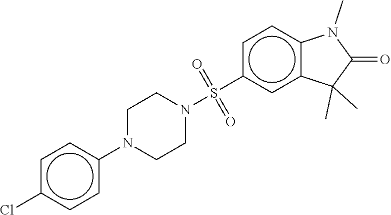 |
| | | | S(═O)(═)(c(ccc(c1C2(C)C)N(C2═O)C)c1)N(CC3)CCN3c4ccc(cc4 [symbol] |
| ChemDiv Targeted Diversity Library | Chem Div | D727- 0524 | 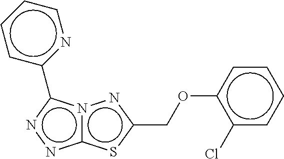 |
| | | | n1(nc(s2)COc3ccccc3Cl)c2nnc1c4ccccn4 |
| ChemDiv Targeted Diversity Library | Chem Div | D727- 0025 | 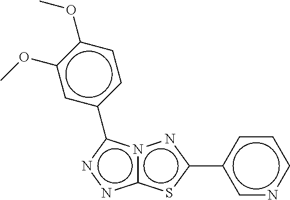 |
| | | | n12c(nnc1c3ccc(c(OC)c3)OC)sc(c(cccn4)c4)n2 |
| ChemDiv Targeted Diversity Librarly | Chem Div | D727- 0838 | 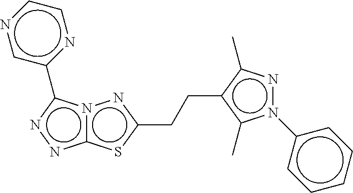 |
| | | | n1(nc(s2)CCc(c(C)nn3c4ccccc4)c3C)c2nnc1c5cnccn5 |
| ChemDiv Targeted Diverstiy Library | Chem Div | 7695- 0166 | 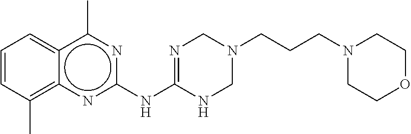 |
| | | | c1(nc(C)c(c2n1)cccc2C)NC3═NCN(CCCN(CC4)CCO4)CN3 |
| ChemDiv Targeted Diversity Library | Chem Div | C200- 7011 | 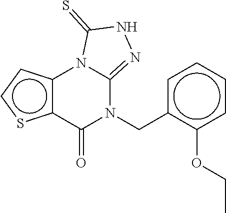 |
| | | | C1(═NNC2═S)N2c(ccs3)c3C(═O)N1Cc4cccc4OCC |
| ChemDiv Targeted Diversity Library | Chem Div | D727- 0743 | 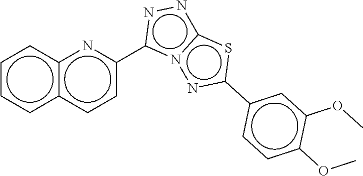 |
| | | | n12c(nnc1c3ccc(c4n3)cccc4)sc(c5ccc(c(OC)c5)OC)n2 |
| ChemDiv Targeted Diversity Library | Chem Div | C200- 9425 | 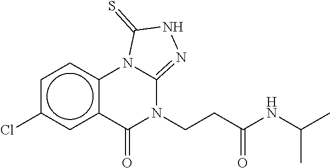 |
| | | | C1(═NNC2═S)N2c3c(cc(cc3)Cl)C(═O)N1CCC(═O)NC(C)C |
| ChemDiv Targeted Diversity Library | Chem Div | C200- 7327 | 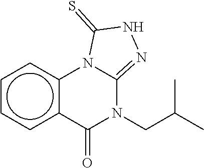 |
| | | | C1(═NNC2═S)N2c(cccc3)c3C(═O)N1CC(C)C |
| ChemDiv Targeted Diversity Library | Chem Div | C200- 8090 | 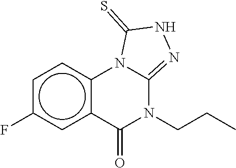 |
| | | | C1(═NNC2═S)N2c3c(cc(cc3)F)C(═O)N1CCC |
| ChemDiv Targeted Diversity Library | Chem Div | C200- 7093 | 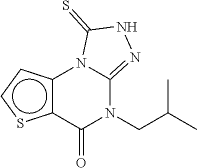 |
| | | | C1(═NNC2═S)N2c(c3C(═O)N1CC(C)C)ccs3 |
| ChemDiv Targeted Diversity Library | Chem Div | D727- 0878 | 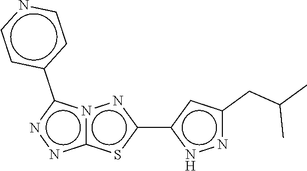 |
| | | | n12c(nnc1c(cc3)ccn3)sc(c4cc(n[nH]4)CC(C))C)n2 |
| ChmDiv Targeted Diversity Library | Chem Div | C301- 7218 | 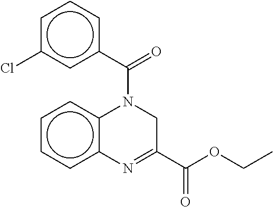 |
| | | | N1(c2c(cccc2)N═C(C(OCC)═O)C1═O)C(═O)c3cccc(Cl)c3 |
| ChemDiv Targeted Diversity Library | Chem Div | D727- 0051 | 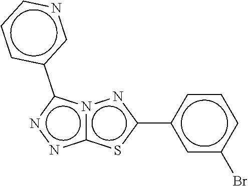 |
| | | | n12c(nnc1c(cccn3)c3)sc(c4cccc(Br)c4)n2 |
| ChemDiv Targeted Diversity Library | Chem Div | E143- 0032 | 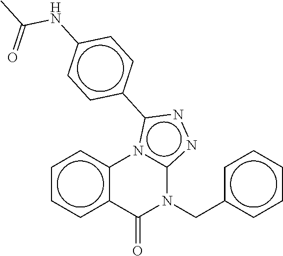 |
| | | | c12n(c(c3C(═O)N1Cc4ccccc4)cccc3)c(nn2)c5ccc(cc5)NC(C)═ [symbol] |
| ChemDiv Targeted Diversity Library | Chem Div | C200- 7326 | 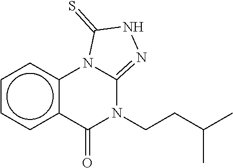 |
| | | | C1(═NNC2═S)N2c(c3C(═O)N1CCC(C)C)cccc3 |
| ChemDiv Targeted Diversity Library | Chem Div | C200- 7834 | 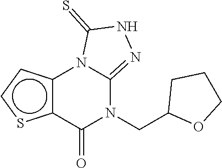 |
| | | | C1(═NNC2═S)N2c(c3C(═O)N1CC4CCCO4)ccs3 |
| ChemDiv Targeted Diversity Library | Chem Div | D727- 768 | 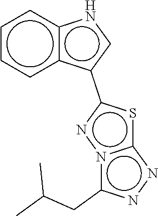 |
| | | | n1(n2)c(nnc1CC(C)C)sc2c3c4c([nH]c3)cccc4 |
| ChemDiv Targeted Diversity Library | Chem Div | D727- 0837 | 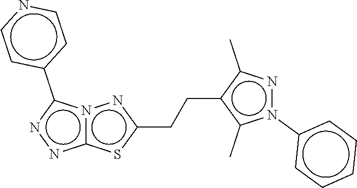 |
| | | | n1(nc(s2)CCc(c(C)nn3c4ccccc4)c3C)c2nnc1c(cc5)ccn5 |
| ChemDiv Targeted Diversity Library | Chem Div | D727- 0740 | 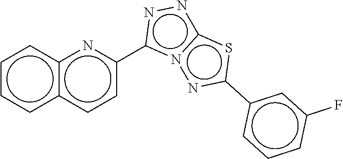 |
| | | | n12c(nnc1c3ccc(c4n3)cccc4)sc(c5cccc(F)c5)n2 |
| ChemDiv Targeted Diversity Library | Chem Div | D588- 0191 | 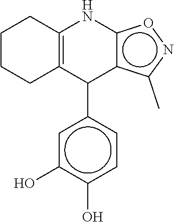 |
| | | | c(o1)(NC(CCCC2═O)═C2C3c4ccc(c(O)c4)O)c3c(n1)C |
| ChemDiv Targeted Diversity Library | Chem Div | D727- 0059 | 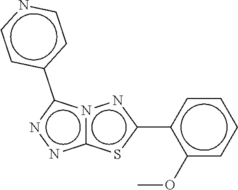 |
| | | | n12c(nnc1c(cccn3)c3)sc(c4ccccc4OC)n2 |
| ChemDiv Targeted Diversity Library | Chem Div | D727- 0796 | 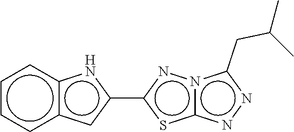 |
| | | | n12c(nnc1CC(C)C)sc(c3cc(c4[nH]3)cccc4)n2 |
| ChemDiv Targeted Diversity Library | Chem Div | D727- 0159 | 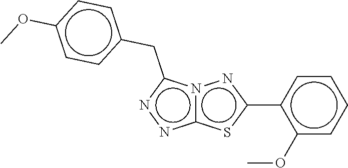 |
| | | | n12c(nnc1Cc(cc3)ccc3OC)sc(c4ccccc4OC)n2 |
| ChemDiv Targeted Diversity Library | Chem Div | D727- 0063 | 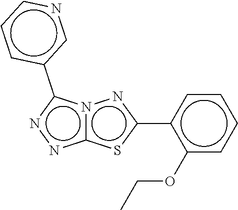 |
| | | | n12c(nnc1c(cccn3)c3)sc(c4ccccc4OCC)n2 |
| ChemDiv Targeted Diversity Library | Chem Div | C200- 7283 | 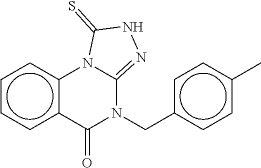 |
| | | | C1(═NNC2═S)N2c(cccc3)c3C(═O)N1Cc(cc4)ccc4C |
| ChemDiv Targeted Diversity Library | Chem Div | D727- 0181 | 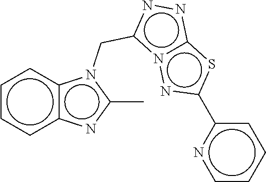 |
| | | | n12c(nnc1Cn(c3c(n4)cccc3)c4C)sc(c5ccccn5)n2 |
| ChemDiv Targeted Diversity Library | Chem Div | D727- 0069 | 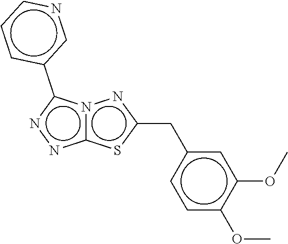 |
| | | | n1(nc(s2)Cc(ccc(c3OC)OC)c3)c2nnc1c(cccn4)c4 |
| ChemDiv Targeted Diversity Library | Chem Div | D727- 0090 | 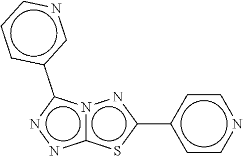 |
| | | | n12c(nnc1c(cccn3)c3)sc(c(cc4)ccn4)n2 |
| ChemDiv Targeted Diversity Library | Chem Div | C200- 9423 | 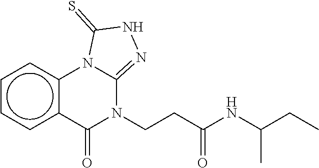 |
| | | | C1(═NNC2═S)N2c(c3C(═O)N1CCC(═O)NC(C)CC)cccc3 |
| ChemDiv Targeted Diversity Library | Chem Div | D727- 0853 | 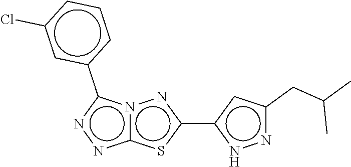 |
| | | | n12c(nnc1c3cccc(Cl)c3)sc(c4cc(n[nH]4)CC(C)C)n2 |
| ChemDiv Targeted Diversity Library | Chem Div | F233- 0200 | 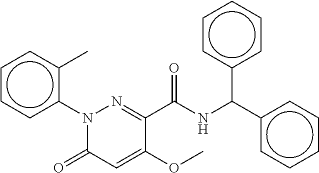 |
| | | | N1(c2ccccc2C)C(═O)C═C(C(C(═O)NC(c3ccccc3)c4ccccc4)═N1) [symbol] |
| ChemDiv Targeted Diversity Library | Chem Div | C200- 7014 | 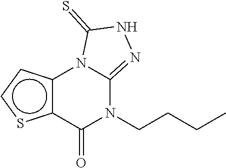 |
| | | | C1(═NNC2═S)N2c(c3C(═)N1CCCC)ccs3 |
| ChemDiv Targeted Diversity Library | Chem Div | E218- 0327 | 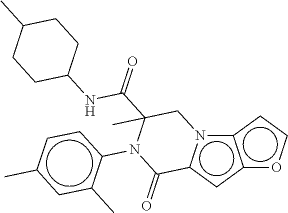 |
| | | | N1(c2ccc(cc2C)C)C(═O)c3n(CC1(C)C(═O)NC4CCC(CC4)C)c5c(occ [symbol] |
| ChemDiv Targeted Diversity Library | Chem Div | D727- 0535 | 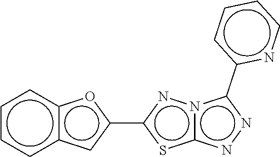 |
| | | | n12c(nnc1c3ccccn3)sc(c4cc(c5o4)cccc5)n2 |
| ChemDiv Targeted Diversity Library | Chem Div | E218- 0181 | 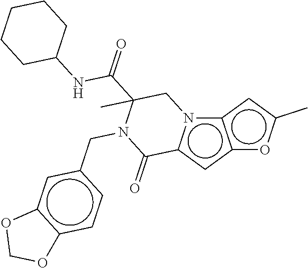 |
| | | | n1(CC2)C)C(═O)NC3CCCCC3)c(cc(oc(C)c4)c14)C(N2Cc(ccc(c56)OCO [symbol] |
| ChemDiv Targeted Diversity Library | Chem Div | D727- 0883 | 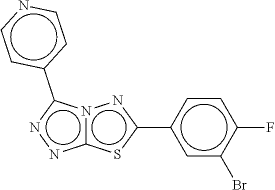 |
| | | | n12c(nnc1c(cc3)ccn3)sc(c4ccc(c(Br)c4)F)n2 |
| ChemDiv Targeted Diversity Library | Chem Div | E922- 0258 | 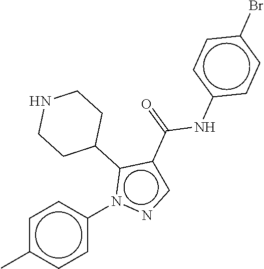 |
| | | | c(cnn1c2ccc(cc2)C)(C(═O)Nc(cc3)ccc3Br)c1C(CC4)CCN4 |
| ChemDiv Targeted Diversity Library | Chem Div | D727- 0489 | 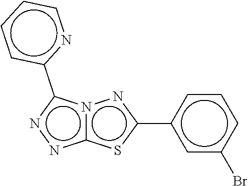 |
| | | | n12c(nnc1c3cccccn3)sc(c4cccc(Br)c4)n2 |
| ChemDiv Targeted Diversity Library | Chem Div | D727- 0619 | 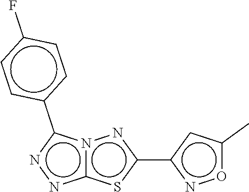 |
| | | | n12c(nnc1c3ccc(cc3)F)sc(c4cc(on4)C)n2 |
| ChemDiv Targeted Diversity Library | Chem Div | D727- 0049 | 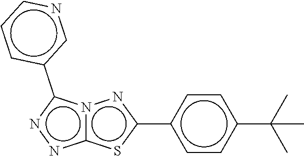 |
| | | | N12c(nnc1c(cccn3)c3)sc(c(cc4)ccc4C(C)(C)C)n2 |
| ChemDiv Targeted Diversity Library | Chem Div | D727- 0047 | 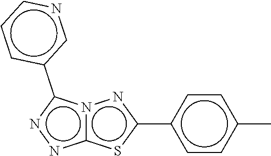 |
| | | | n12c(nnc1c(cccn3)c3)sc(c4ccc(cc4)C)n2 |
| ChemDiv Targeted Diversity Library | Chem Div | D727- 0912 | 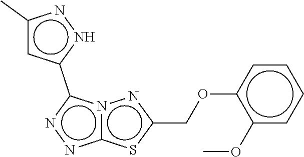 |
| | | | n1(nc(s2)COc3ccccc3OC)c2nnc1c4cc(n[nH]4)C |
| ChemDiv Targeted Diversity Library | Chem Div | D727- 0754 | 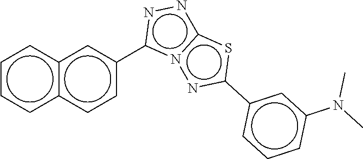 |
| | | | n12c(nnc1c3ccc(c4n3)cccc4)sc(c5cccc(N(C)C)c5)n2 |
| ChemDiv Targeted Diversity Library | Chem Div | E234- 0004 | 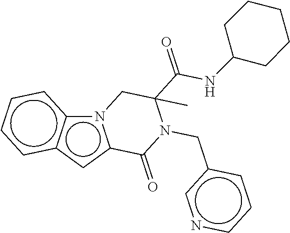 |
| | | | N(Cc(cccn1)c1)(C2═O)C(CO)(Cn(c3c(cccc3)c4)c24)C(═O)NC5CCC [symbol] |
| ChemDiv Targeted Diveristy Library | Chem Div | E218- 0329 | 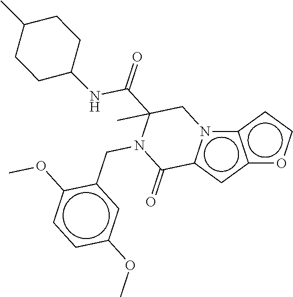 |
| | | | N1(c2c(OC)ccc(OC)c2)C(═O)c3n(CC1(C)C(═O)NC4CCC(CC4C)c5c( [symbol] |
| ChemDiv Targeted Diversity Library | Chem Div | D727- 0713 | 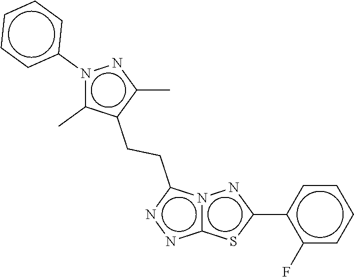 |
| | | | n1(c(CCc(c(C)nn2c3ccccc3)c2C)nn4)c4sc(c5ccccc5F)n1 |
| ChemDiv Targeted Diversity Library | Chem Div | D588- 0192 | 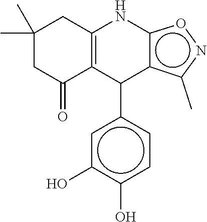 |
| | | | c(o1)(NC(CC(C)(C)CC2═O)═C2C3c4ccc(c(O)c4)O)c3c(N1)C |
| ChemDiv Targeted Diversity Library | Chem Div | C200- 7463 | 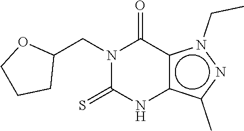 |
| | | | c1(C2═O)c(c(nn1CC)C)NC(═S)N2CC3CCCO3 |
| ChemDiv Targeted Diversity Library | Chem Div | D243- 0426 | 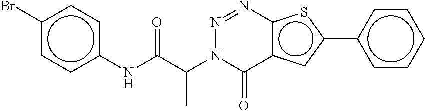 |
| | | | N(C(C)C(═O)Nc(cc1)ccc1Br)(C2═O)N═Nc(sc(c3ccccc3)c)c24 [symbol] |
| ChmDiv Targeted Diversity Library | Chem Div | D727- 0536 | 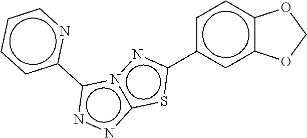 |
| | | | n12c(nnc1c3ccccn3)sc(c(ccc(c45)OCO4)c5)n2 |
| ChemDiv Targeted Diversity Library | Chem Div | C066- 3867 | 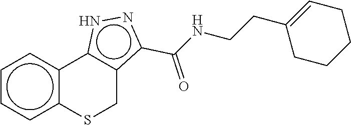 |
| | | | c1(CSc(c23)cccc2)c3[nH]nc1C(NCCC4═CCCCC4)═O |
| ChemDiv Targeted Diversity Library | Chem Div | D727- 0055 | 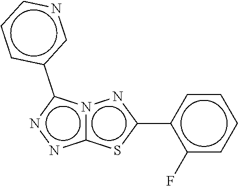 |
| | | | n12c(nnc1c(cccn3)c3)sc(c4ccccc4F)n2 |
| ChemDiv Targeted Diversity Library | Chem Div | D727- 0119 | 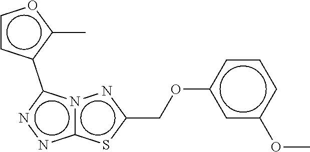 |
| | | | n1(nc(s2)COc3cccc(OC)c3)c2nnc1c4ccoc4C |
| ChemDiv Targeted Diversity Library | Chem Div | C200- 8885 | 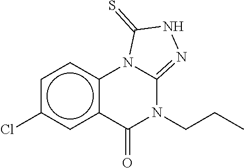 |
| | | | C1(═NNC2═S)N2c3c(cc(cc3)Cl)C(═O)N1CCC |
| ChemDiv Targeted Diversity Library | Chem Div | D727- 0755 | 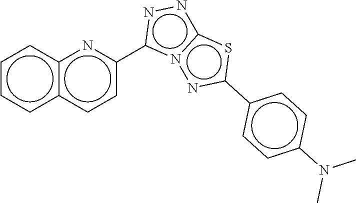 |
| | | | n12c(nnc1c3ccc(c4n3)cccc4)sc(c(cc5)ccc5N(C)C)n2 |
| ChemDiv Targeted Diversity Library | Chem Div | F305- 0030 | 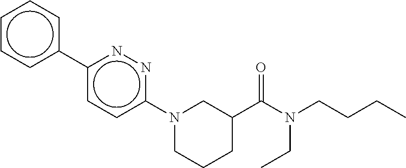 |
| | | | C(CCCN1c2ccc(nn2)c3ccccc3)(C1)C(═O)N(CCCC)CC |
| ChemDiv Targeted Diversity Library | Chem Div | D727- 0772 | 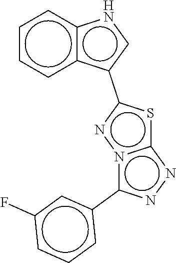 |
| | | | n1(n2)c(nnc1c3cccc(F)c3)sc2c4c5c([nH]c4)cccc5 |
| ChemDiv Targeted Diversity Library | Chem Div | D727- 0884 | 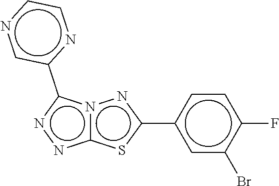 |
| | | | n12c(nnc1c3cnccn3)sc(c4ccc(c(Br)c4)F)n2 |
| ChemDiv Targeted Diversity Library | Chem Div | D727- 0852 | 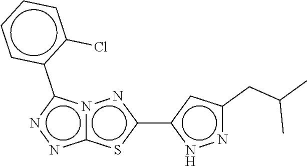 |
| | | | n12c(nnc1c3ccccc3Cl)sc(c4cc(n[nH]4)cc(C)C)n2 |
| ChemDiv Targeted Diversity Library | Chem Div | C301- 8999 | 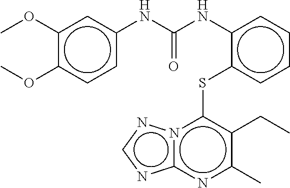 |
| | | | n1(ncn2)c2nc(c(CC)c1Sc3ccccc3NC(═O)Nc4ccc(c(OC)c4)OC) [symbol] |
| ChemDiv Targeted Diversity Library | Chem Div | E218- 0201 | 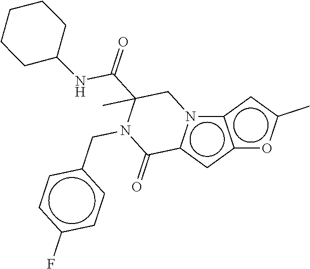 |
| | | | n1(CC2(C)C(═O)NC3CCCCC3)c(cc(oc(C)c4)c14)C(N2Cc(cc5)cc5 [symbol] |
| ChemDiv Targeted Diversity Library | Chem Div | C201- 1864 | 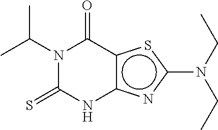 |
| | | | c(sc(n1)N(CC)CC)(C2═O)c1NC(═S)N2C(C)C |
| ChemDiv Targeted Diversity Library | Chem Div | C200- 2668 | 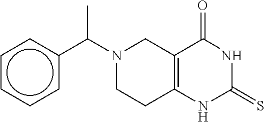 |
| | | | C(C1)(═C(CCN1C(C)c2ccccc2)NC(N3)═S)C3═O |
| ChemDiv Targeted Diversity Library | Chem Div | D727- 0165 | 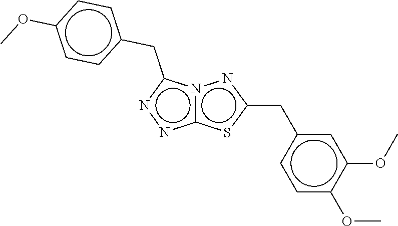 |
| | | | n1(nc(s)Cc(ccc(c3OC)OC)c3)c2nnc1Cc(cc4)ccc4OC |
| ChemDiv Targeted Diversity Library | Chem Div | C200- 4690 | 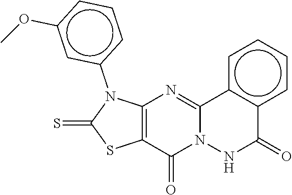 |
| | | | C12═C(SC(═S)N1c3cccc(OC)c3)C(N4C(c5c(cccc5)C(N4)═O)═N2) [symbol] |
| ChemDiv Targeted Diversity Library | Chem Div | D727- 0088 | 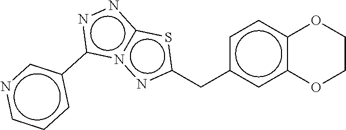 |
| | | | n1(nc(s2)Cc(ccc(c34)OCCO3)c4)c2nnc1c(cccn5)c5 |
| ChemDiv Targeted Diversity Library | Chem Div | D727- 0786 | 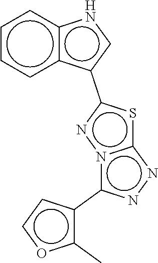 |
| | | | n1(n2)c(nnc1c3ccoc3C)sc2c4c5c([nH]c4)cccc5 |
| ChemDiv Targeted Diversity Library | Chem Div | C301- 9367 | 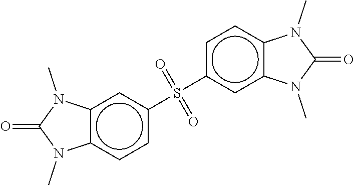 |
| | | | C1(═O)N(C)c(c2N1C)ccc(c2)S(═O)(═O)c(ccc(c3N4C)N(C4═O)C) [symbol] |
| ChemDiv Targeted Diversity Library | Chem Div | D087- 0518 | 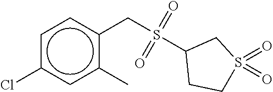 |
| | | | S(═O)(═O)Nc1ccc(cc1C)Cl)C(CCS2(═O)═O)C2 |
| ChemDiv Targeted Diversity Library | Chem Div | D727- 0857 | 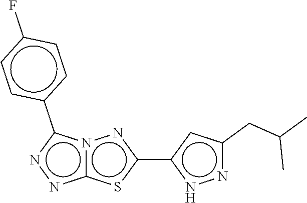 |
| | | | n12c(nnc1c3ccc(cc3)F)sc(c4cc(n[nH]4)CC(C)C)n2 |
| ChemDiv Targeted Diversity Library | Chem Div | C200- 9422 | 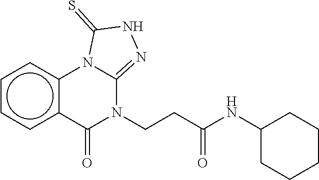 |
| | | | C1(═NNC2═S)N2c(c3C(═O)N1CCC(═O)NC4CCCCC4)cccc3 |
| ChemDiv Targeted Diversity Library | Chem Div | ES34- 0255 | 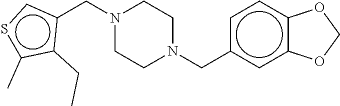 |
| | | | c1(CN(CC2)CCN2Cc(ccc(c34)OCO3)c4)csc(C)c1CC |
| ChemDiv Targeted Diversity Library | Chem Div | F083- 0005 | 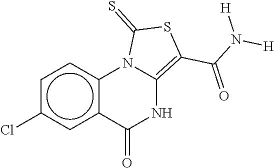 |
| | | | N1(c2c(cc(Cl)cc2)C(N3)═O)C3═C(SC1═S)C(N([H])[H])═O |
| ChemDiv Targeted Diversity Library | Chem Div | D715- 2437 | 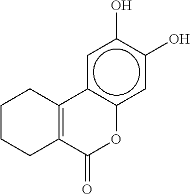 |
| | | | C1(═C2CCCC1)c3c(OC2═O)cc(O)c(O)c3 |
| ChemDiv Targeted Diversity Library | Chem Div | C301- 9375 | 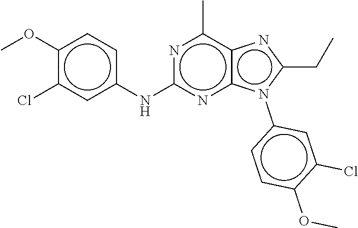 |
| | | | c12c(c(nc(Nc(ccc(c3Cl)OC)c3)n1)C)nc(CC)n2c4ccc(c(Cl)c4)O [symbol] |
| ChemDiv Targeted Diversity Library | Chem Div | C200- 7329 | 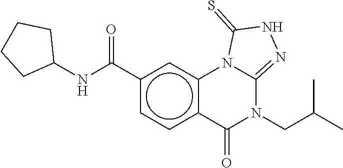 |
| | | | C1(═NNC2═S)N2c3c(C(═O)N1CC(C)C)ccc(c3)C(═)NC4CCCC [symbol] |
| ChemDiv Targeted Diversity Library | Chem Div | D727- 0717 | 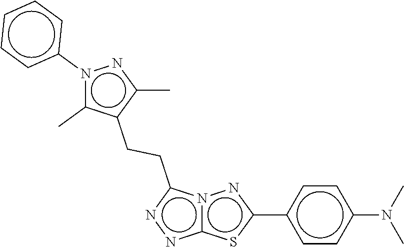 |
| | | | n1(c(CCc(c(C)nn2c3ccccc3)c2C)nn4)c4sc(c(cc5)ccc5N(C)C)n [symbol] |
| ChemDiv Targeted Diversity Library | Chem Div | D727- 0742 | 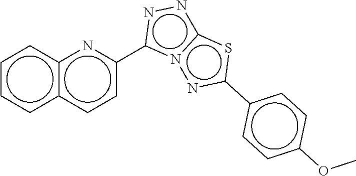 |
| | | | n12c(nnc1c3ccc(c4n3)cccc4)sc(c5ccc(cc5)OC)n2 |
| ChemDiv Targeted Diversity Library | CHem Div | E234- 0018 | 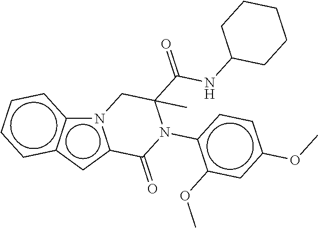 |
| | | | N1(c2ccc(cc2OC)OC)C(═O)c3n(CC1(C)C(═O)NC4CCCCC4)c5c(ccc [symbol] |
| ChemDiv Targeted Diversity Library | Chem Div | F260- 0258 | 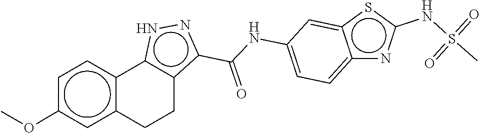 |
| | | | c1(C(═O)Nc(ccc(c2c3)nc3NS(C)(═O)═O)c2)n[nH]c4c1CCc(c45)cc( [symbol] |
| ChemDiv Targeted Diversity Library | Chem Div | D727- 0828 | 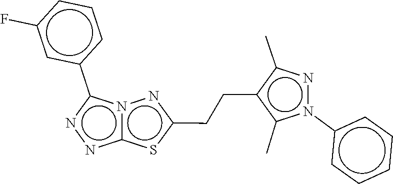 |
| | | | n1(nc(s2)CCc(c(C)nn3c4ccccc4)c3C)c2nnc1c5cccc(F)c5 |
| ChemDiv Targeted Diversity Library | Chem Div | D727- 0089 | 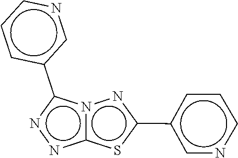 |
| | | | n12c(nnc1c(cccn3)c3)sc(c(cccn4)c4)n2 |
| ChemDiv Targeted Diversity Library | Chem Div | D727- 0890 | 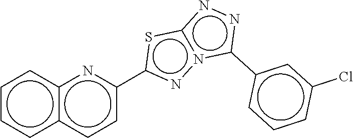 |
| | | | n12c(nnc1c3cccc(Cl)c3)sc(c4ccc(c5n4)cccc5)n2 |
| ChemDiv Targeted Diversity Library | Chem Div | D727- 0712 | 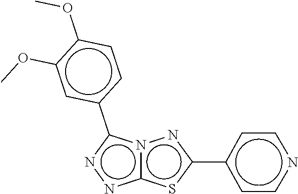 |
| | | | n1(c(CCc(ccc(c2OC)OC)c2)nn3)c3sc(c(cc4)ccn4)n1 |
| ChemDiv Targeted Diversity Library | Chem Div | F233- 0181 | 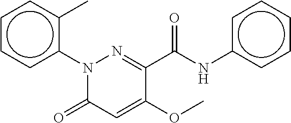 |
| | | | N1(c2ccccc2C)C(═O)C═C(C(C(═O)Nc3ccccc3)═N1)OC |
| ChemDiv Targeted Diversity Library | Chem Div | D433- 1829 | 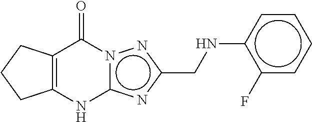 |
| | | | n1(nc(n2)CNc3ccccc3F)c2NC(CCC4)═C4C1═O |
| ChemDiv Targeted Diversity Library | Chem Div | D727- 0829 | 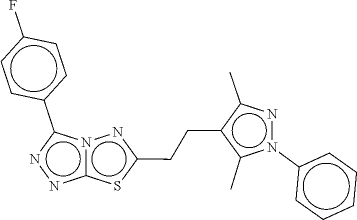 |
| | | | n1(nc(s2)CCc(c(C)nn3c4ccccc4)c3C)c2nnc1c5ccc(cc5)F |
| ChemDiv Targeted Diversity Library | Chem Div | F083- 0017 | 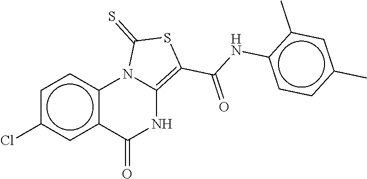 |
| | | | N1(c2c(cc(Cl)cc2)C(N3)═O)C3═C(SC1═S)C(═O)Nc4ccc(cc4C) [symbol] |
| ChemDiv Targeted Diversity Library | Chem Div | C243- 0026 | 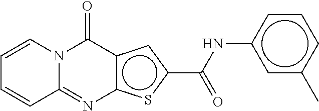 |
| | | | c1(c2═O)c(sc(C(═O)Nc3cccc(C)c3)c1)N═C(C═CC═C4)N24 |
| ChemDiv Targeted Diversity Library | Chem Div | F083- 0404 | 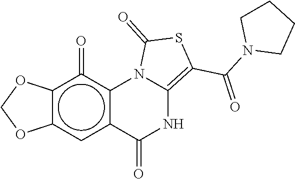 |
| | | | C1(═C(C(═O)N2CCCC2)SC3═S)N3c4c(cc(c5c4)OCO5)C(═O)N [symbol] |
| ChemDiv Targeted Diversity Library | Chem Div | F233- 0420 | 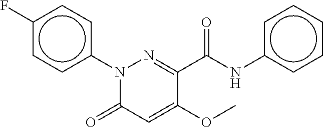 |
| | | | N1═C(C(═O)Nc2ccccc2)C(═CC(═O)N1c3ccc(cc3)F)OC |
| ChemDiv Targeted Diversity Library | Chem Div | D727- 0123 | 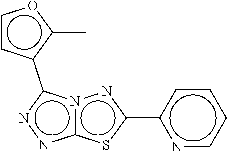 |
| | | | n12c(nnc1c3ccoc3C)sc(c4ccccn4)n2 |
| Enamine 1 | Enamine | T0510- 8045 | 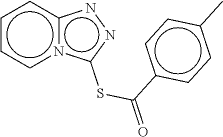 |
| | | | Cc1ccc(cc1)C(═O)Sc1nnc2ccccn12 |
| ChemDiv Targeted Diversity Library | Chem Div | D097- 0031 | 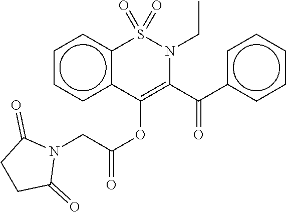 |
| | | | S(═O)(═O)(c(cccc1)c1C(═C2C(c3ccccc3)═O)OC(═O)Cn4C(═O)CCC4═ [symbol] |
| ChemDiv Targeted Diversity Library | Chem Div | D297- 0031 | 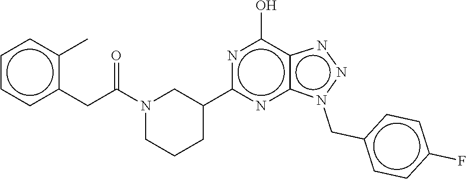 |
| | | | c12c(c(nc(C(CCCN3C(═O)Cc4ccccc4C)C3)n1)O)nnn2Cc(cc5)cc [symbol] |
| ChemDiv Targeted Diversity Library | Chem Div | E234- 0008 | 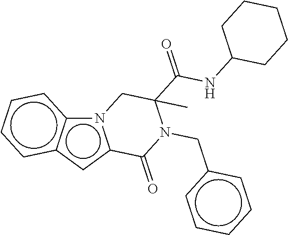 |
| | | | N(Cc1ccccc1)(C2═O)C(C)(Cn(c3c(cccc3)c4)c24)C(═O)NC5CCCC [symbol] |
| ChemDiv Targeted Diversity Library | Chem Div | E157- 3455 | 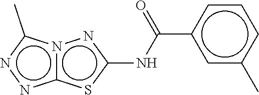 |
| | | | n(c(C)nn1)(n2)c1sc2NC(═O)c3cccc(C)c3 |
| Enamine 1 | Enamine | T0502- 0200 | 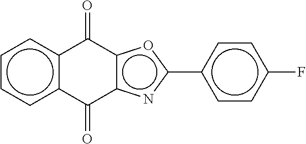 |
| | | | Fc1ccc(cc1)c1nc2C(═O)c3ccccc3C(═O)c2o1 |
| ChemDiv Targeted Diversity Library | Chem Div | D421- 0876 | 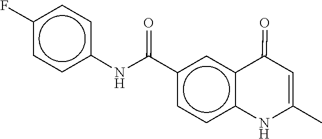 |
| | | | c12c(ccc(c1)C(═O)Nc(cc3)ccc3F)NC(═CC2═O)C |
| ChemDiv Targeted Diversity Library | Chem Div | D727- 0072 | 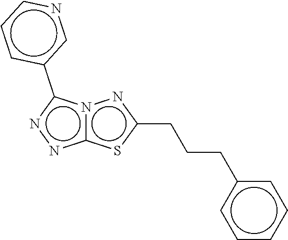 |
| | | | n1(nc(s2)CCCc3ccccc3)c2nnc1c(cccn4)c4 |
| ChemDiv Targeted Diversity Library | Chem Div | D727- 0845 | 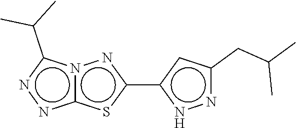 |
| | | | n12c(nnc1C(C)C)sc(c3cc(n[nH]3)CC(C)C)n2 |
| ChemDiv Targeted Diversity Library | Chem Div | D656- 0061 | 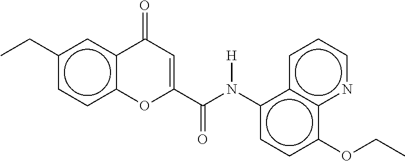 |
| | | | C(C(═O)N([H])c1ccc(c2c1cccn2)OCC)(Oc(ccc(c3)CC)c3C4═O)═ [symbol] |
| ChemDiv Targeted Diversity Library | Chem Div | F255- 0057 | 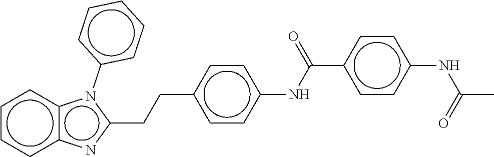 |
| | | | c1(CCc(cc2)ccc2NC(═O)c3ccc(cc3)NC(C)═O)nc(c4n1c5ccccc5)c [symbol] |
| ChemDiv Targeted Diversity Library | Chem Div | E218- 0232 | 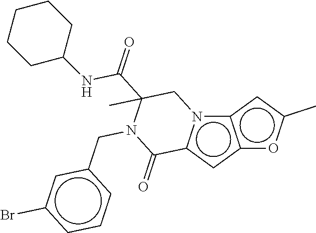 |
| | | | n1(CC2(C)C(═O)NC3CCCCC3)c(cc(oc(C)c4)c14)C(N2Cc5cccc(Br) [symbol] |
| ChemDiv Targeted Diversity Library | Chem Div | F293- 0762 | 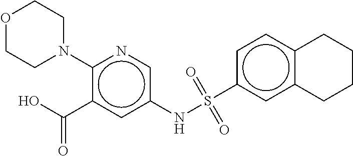 |
| | | | S(═O(═O)(c1ccc(c2c1)CCCC2)Nc(cnc(c3C(O)═O)N(CC4)CCO4) |
| ChemDiv Targeted Diversity Library | Chem Div | F305- 0129 | 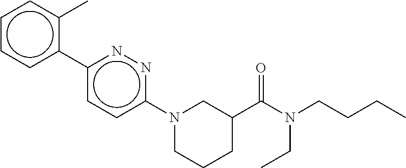 |
| | | | C(CCCN1c2ccc(nn2)c3ccccc3C)(C1)C(═O)N(CCCC(CC |
| ChemDiv Targeted Diversity Library | Chem Div | D727- 0824 | 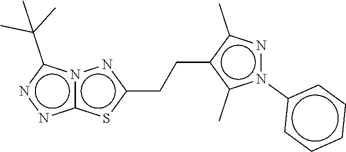 |
| | | | n1(nc(s2)CCc(c(C)nn3c4ccccc4)c3C)c2nnc1C(C)(C)C |
| ChemDiv Targeted Diversity Library | Chem Div | C301- 7136 | 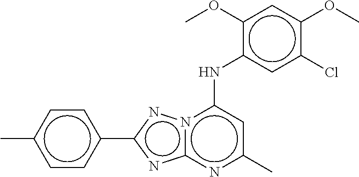 |
| | | | n1(nc(n2)c3ccc(cc3)C)c2nc(C)cc1Nc4c(OC)cc(c(Clc4)OC |
| ChemDiv Targeted Diversity Library | Chem Div | E218- 0397 | 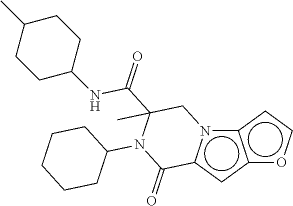 |
| | | | C(C)(C1)(C(═O)NC2CCC(CC2)C)N(C3CCCCC3)C(c4n1cSc(c4)occ5 [symbol] |
| ChemDiv Targeted Diversity Library | Chem Div | D390- 0881 | 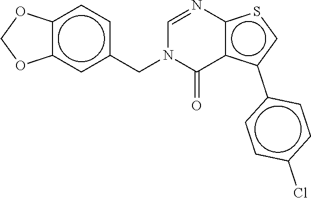 |
| | | | c12c(scc1c3ccc(cc3)Cl)N═CN(Cc(ccc(c45)OCO4)c5)C2═O |
| ChemDiv Targeted Diversity Library | Chem Div | D727- 0819 | 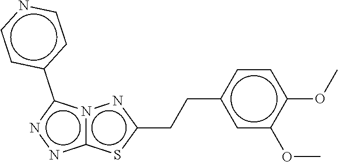 |
| | | | n1(nc(s2)CCc(ccc(c3OC)OC)c3)c2nnc1c(cc4)ccn4 |
| ChemDiv Targeted Diversity Library | Chem Div | D316- 0527 | 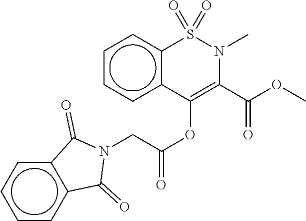 |
| | | | S1(═O)(═O)c(cccc2)c2C(═C(C(OC)═O)N1C)OC(═O)CN3C(═O)c(c4C3═ [symbol] |
| ChemDiv Targeted Diversity Library | Chem Div | D727- 0714 | 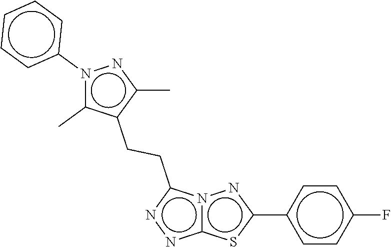 |
| | | | n1(c(CCc(c(C)nn2c3ccccc3)c2C)nn4)c4sc(c5ccc(cc5)F)n1 |
| ChemDiv Targeted Diversity Library | Chem Div | E667- 0223 | 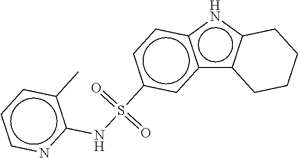 |
| | | | S(═O)(═O)(c(ccc1c2c3c([nH]1)CCCC3)c2)Nc4c(cccn4)C |
| ChemDiv Targeted Diversity Library | Chem Div | F305- 0061 | 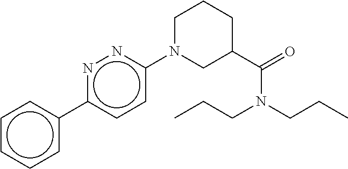 |
| | | | C(CCCN1c2ccc(nn2)c3ccccc3)(C1)C(═O)N(CCC)CCC |
| ChemDiv Targeted Diversity Library | Chem Div | D686- 0195 | 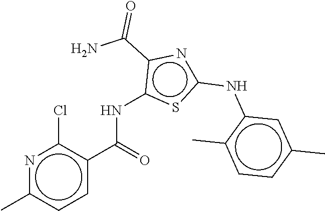 |
| | | | c1(NC(═O)c2ccc(nc2Cl)C)sc(nc1C(N)═O)Nc3cc(C)ccc3C |
| ChemDiv Targeted Diversity Library | Chem Div | D727- 0071 | 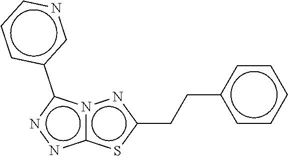 |
| | | | n1(nc(s2)CCc3ccccc3)c2nnc1c(cccn4)c4 |
| ChemDiv Targeted Diversity Library | Chem Div | D664- 0047 | 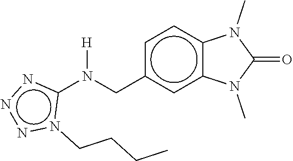 |
| | | | C1(═O)N(C)c2c(cc(cc2)CN([H])c3nnnn3CCCC)N1C |
| ChemDiv Targeted Diversity Library | Chem Div | D727- 0746 | 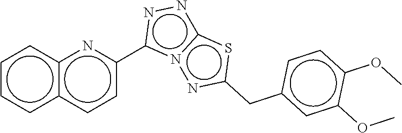 |
| | | | n1(nc(s2)Cc(ccc(c3OC)OC)c3)c2nnc1c4ccc(c5n4)cccc5 |
| ChemDiv Targeted Diversity Library | Chem Div | F305- 0036 | 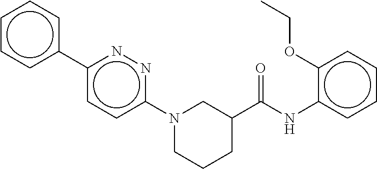 |
| | | | N(CCCC1C(═O)Nc2ccccc2OCC)(C1)c3ccc(nn3)c4ccccc4 |
| ChemDiv Targeted Diversity Library | Chem Div | F083- 0285 | 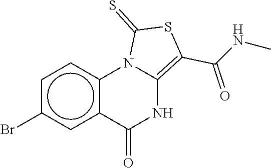 |
| | | | N1(c2c(cc(Br)cc2)C(N3)═O)C3═C(SC1═S)C(NC)═O |
| ChemDiv Targeted Diversity Library | Chem Div | D278- 0547 | 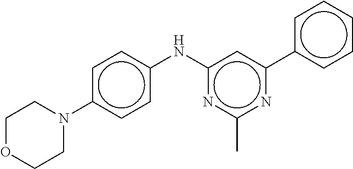 |
| | | | c1(Nc(cc2)ccc2N(CC3)CCO3)cc(c4ccccc4)nc(C)n1 |
| ChemDiv Targeted Diversity Library | Chem Div | D087- 0519 | 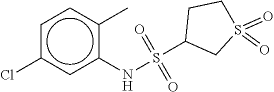 |
| | | | S(═O(═O)(Nc1c(C)ccc(Cl)c1)C(CCS2(═O)═O)C2 |
| ChemDiv Targeted Diversity Library | Chem Div | F083- 0416 | 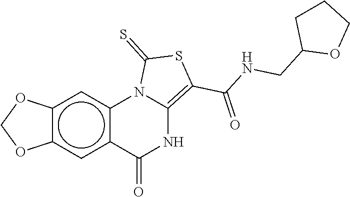 |
| | | | N1(c2c(cc(OCO3)c3c2)C(N4)═O)C4═C(SC1═S)C(═O)NCC5CCC [symbol] |
| ChemDiv Targeted Diversity Library | Chem Div | D585- 0166 | 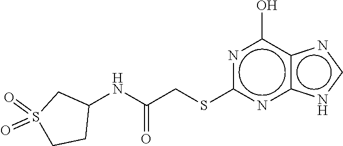 |
| | | | S1(CCC(C1)NC(CSc2nc(O)c3c([nH]cn3)n2)═O)(═O)═O |
| ChemDiv Targeted Diversity Library | CHem Div | D305- 1386 | 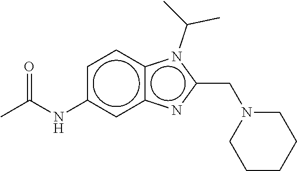 |
| | | | c1(CN2CCCCC2)n(C(C)C)c(c3n1)ccc(c3)NC(C)═O |
| ChemDiv Targeted Diversity Library | Chem Div | D513- 3628 | 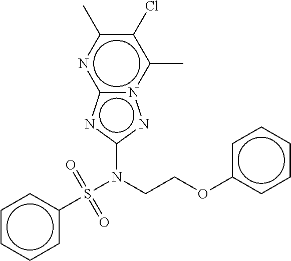 |
| | | | c(N(CCOc1ccccc1)S(c2ccccc2)(═O)═O)(nn3c4nc(c(Cl)c3C)C)n [symbol] |
| ChemDiv Targeted Diversity Library | Chem Div | D212- 0373 | 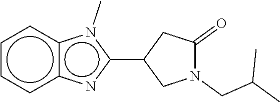 |
| | | | c1(C(CC(N2CC(C)C)═O)C2)n(C)c3c(cccc3)n1 |
| ChemDiv Targeted Diversity Library | Chem Div | D398- 0910 | 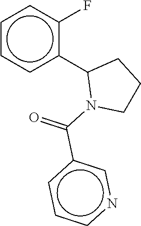 |
| | | | N1(CCCC1c2ccccc2F)C(═O)c(cccn3)c3 |
| ChemDiv Targeted Diversity Library | Chem Div | D132- 0053 | 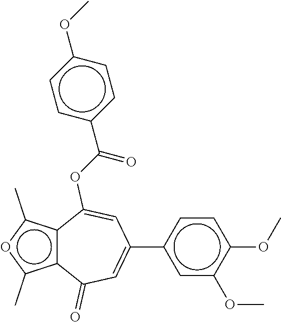 |
| | | | c12c(C(═O)C═C(c3ccc(c(OC)c3)OC)C═C1OC(═O)c4ccc(cc4)OC)c(o [symbol] |
| ChemDiv Targeted Diversity Library | Chem Div | D715- 2438 | 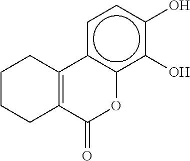 |
| | | | c12c(ccc(O)c1O)c3═C(C(═O)O2)CCCC3 |
| ChemDiv Targeted Diversity Library | Chem Div | E234- 0006 | 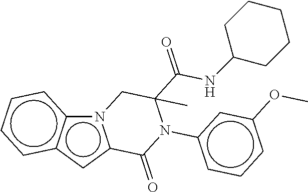 |
| | | | C1(C)(Cn(c2c(c3)cccc2)c3C(═O)N1c4cccc(OC)c4)C(═O)NC5CCC [symbol] |
| ChemDiv Targeted Diversity Library | Chem Div | E218- 0324 | 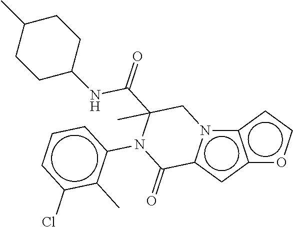 |
| | | | N1(c2cccc(cl)c2C)C(═O)c3n(CC1(C)C(═O)NC4CCC(CC4)C)c5c(oc [symbol] |
| ChemDiv Targeted Diversity Library | Chem Div | C200- 7262 | 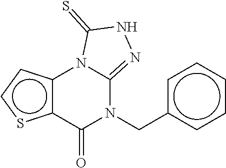 |
| | | | C1(═NNC2═S)N2c(c3C(═O)N1Cc4ccccc4)ccs3 |
| ChemDiv Targeted Diversity Library | Chem Div | F388- 0026 | 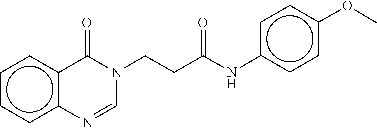 |
| | | | C1(═O)c2c(cccc2)N═CN1CCC(═O)Nc(cc3)ccc3OC |
| ChemDiv Targeted Diversity Library | Chem Div | F401- 0259 | 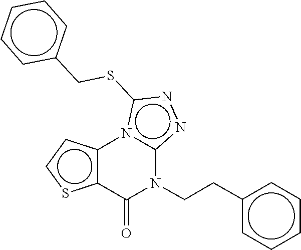 |
| | | | c12n(c(nn1)SCc3ccccc3)c(c4C(═O)N2CCc5ccccc5)ccs4 |
| ChemDiv Targeted Diversity Library | Chem Div | F388- 0145 | 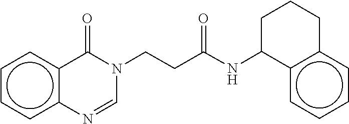 |
| | | | C1(═O)c2c(cccc2)N═CN1CCC(═O)NC3CCCc(cccc4)c34 |
| ChemDiv Targeted Diversity Library | Chem Div | F388- 0151 | 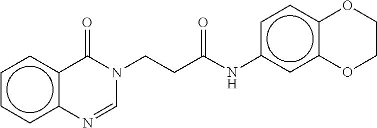 |
| | | | C1(═O)c2c(cccc2)N═CN1CCC(═O)Nc(ccc(c34)OCCO3)c4 |
| ChemDiv Targeted Diversity Library | Chem Div | F407- 012 | 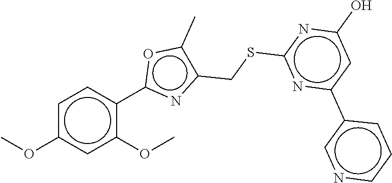 |
| | | | c1(oc(c(CSc2nc(c(cccn3)c3)cc(O)n2)n1)C)c4ccc(cc4OC)OC |
| ChemDiv Targeted Diveristy Library | Chem Div | F449- 1274 | 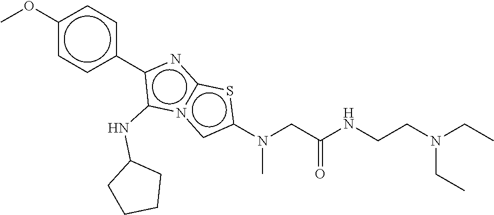 |
| | | | n12c(sc(N(C)CC(NCCN(CC)CC)═O)n1)nc(c3ccc(cc3)OC)c2NC4CC [symbol] |
| ChemDiv Targeted Diversity Library | Chem Div | F458- 0083 | 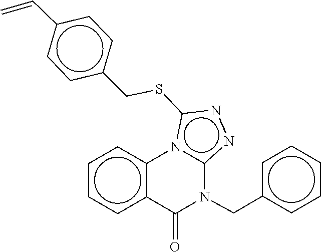 |
| | | | c12n(c(nn1)SCc(cc3)ccc3C═C)c(cccc4)c4C(═O)N2Cc5ccccc5 [symbol] |
| ChemDiv Targeted Diversity Library | Chem Div | F500- 0433 | 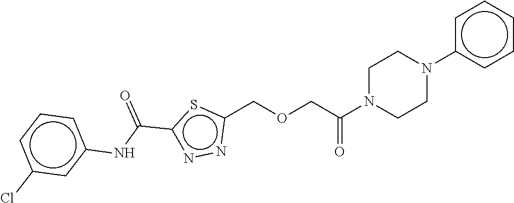 |
| | | | c1(C(═O)Nc2cccc(Cl)c2)sc(nn1)COCC(═O)N(CC3)CCN3c4cccc [symbol] |
| ChemDiv Targeted Diversity Library | Chem Div | F518- 0014 | 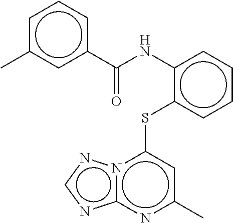 |
| | | | n1(ncn2)c2nc(C)cc1Sc3ccccc3NC(═O)c4cccc(C)c4 |
| ChemDiv Targeted Diversity Library | Chem Div | F518- 0049 | 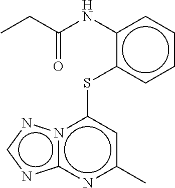 |
| | | | n1(ncn2)c2nc(C)cc1Sc3ccccc3NC(CC)═O |
| ChemDiv Targeted Diversity Library | Chem Div | F518- 0008 | 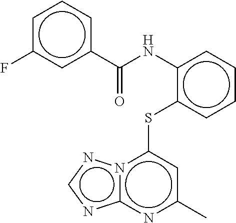 |
| | | | n1(ncn2)c2nc(C)cc1Sc3ccccc3NC(═O)c4cccc(F)c4 |
| ChemDiv Targeted Diversity Library | Chem Div | F542- 0424 | 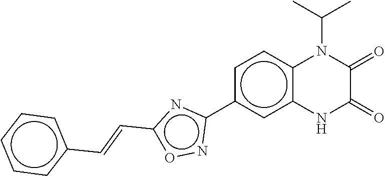 |
| | | | N1(C(C)C)c(cc2)c(cc2c3noc(\C═C\c4ccccc4)n3)NC(═O)C1═O [symbol] |
| ChemDiv Targeted Diversity Library | Chem Div | F545- 0052 | 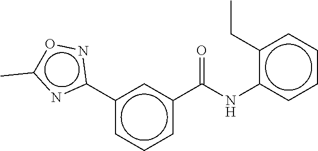 |
| | | | n1c(C)onc1c(cccc2C(═O)Nc3ccccc3CC)c2 |
| ChemDiv Targeted Diversity Library | Chem Div | F571- 0001 | 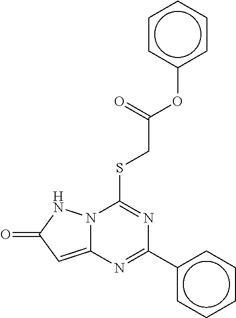 |
| | | | N12C(═CC(═O)N1)N═C(c3ccccc3)N═C2SCC(═O)Oc4ccccc4 |
| ChemDiv Targeted Diversity Library | Chem Div | F571- 0021 | 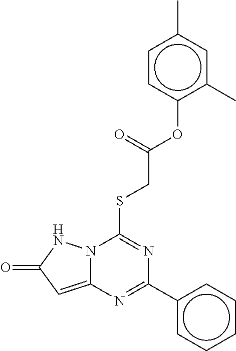 |
| | | | N12C(═CC(═O)N1)N═C(c3ccccc3)N═C2SCC(═O)Oc4ccc(cc4C) [symbol] |
| ChemDiv Targeted Diversity Library | Chem Div | F585- 0060 | 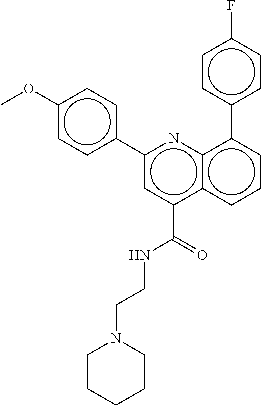 |
| | | | c12c(cccc1c3ccc(cc3)F)c(C(NCCN4CCCCC4)═O)cc(c5ccc(cc5)O [symbol] |
| ChemDiv Targeted Diversity Library | Chem Div | F585- 0086 | 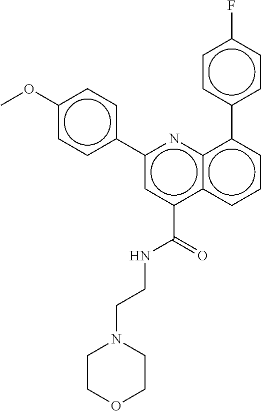 |
| | | | c12c(cccc1c3ccc(cc3)F)c(C(NCCN(CC4)CCO4)═O)cc(c5ccc(cc5)O [symbol] |
| ChemDiv Targeted Diversity Library | Chem Div | F571- 0419 | 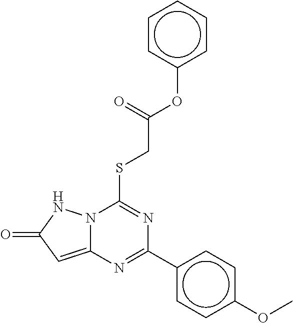 |
| | | | N12C(═CC(═O)N1)N═C(c3ccc(cc3)OC)N═C2SCC(═O)Oc4ccccc [symbol] |
| ChemDiv Targeted Diversity Library | Chem Div | F617- 0185 | 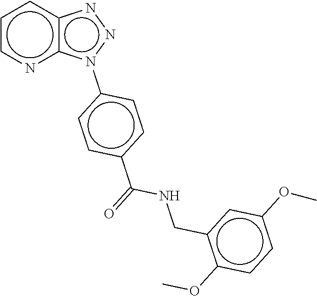 |
| | | | n1(c(cc2)ccc2C(═O)NCc3cc(OC)ccc3OC)c4c(nn1)cccn4 |
| ChemDiv Targeted Diversity Library | Chem Div | F646- 0578 | 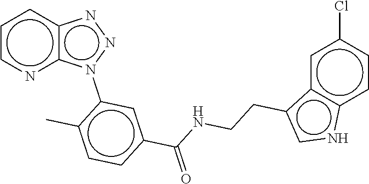 |
| | | | n1(c2c(C)ccc(c2)C(NCCc(c3)c4c([nH]3)ccc(Cl)c4)═O)c5c(nn1)cc [symbol] |
| ChemDiv Targeted Diversity Library | Chem Div | F646- 0636 | 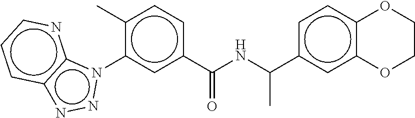 |
| | | | n1(c2c(C)ccc(c2)C(═O)NC(C)c(ccc(c34)OCCO3)c4)c5c(nn1)ccc [symbol] |
| ChemDiv Targeted Diversity Library | Chem Div | F640- 0126 | 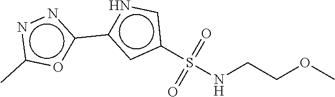 |
| | | | S(NCCOC)(═O)(═O)c(c[nH](c1c2oc(nn2)C)c1 |
| ChemDiv Targeted Diversity Library | Chem Div | F685- 1206 | 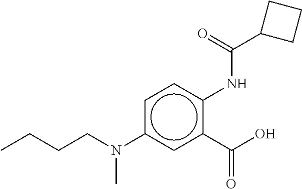 |
| | | | c1(C(O)═O)cc(ccc1NC(C(C2)CC2)═O)N(C)CCCC |
| ChemDiv Targeted Diversity Library | Chem Div | F687- 1038 | 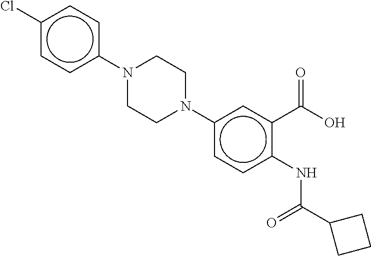 |
| | | | c1(C(O)═O)cc(ccc1NC(C(C2)CC2)═O)N(CC3)CCN3c4ccc(cc4) [symbol] |
| ChemDiv Targeted Diversity Library | Chem Div | F686- 0287 | 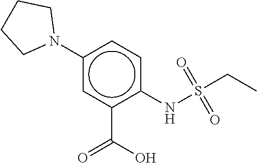 |
| | | | S(CC)(═O)(═O)Nc1ccc(cc1C(O)═O)N2CCCC2 |
| ChemDiv Targeted Diversity Library | Chem Div | F685- 0939 | 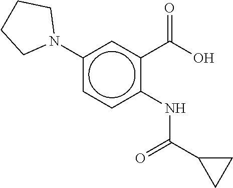 |
| | | | c1(C(O)═O)cc(ccc1NC(═O)C2CC2)N3CCCC3 |
| ChemDiv Targeted Diversity Library | Chem Div | F688- 0002 | 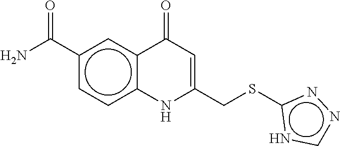 |
| | | | c12c(ccc(c1)C(N)═O)NC(═CC2═O)CSc3nnc[NH]3 |
| ChemDiv Targeted Diversity Library | Chem Div | F680- 0173 | 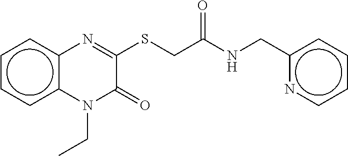 |
| | | | N1(CC)c2c(cccc2)N═C(SCC(═O)NCc3ccccn3)C1═O |
| ChemDiv Targeted Diversity Library | Chem Div | F684- 0019 | 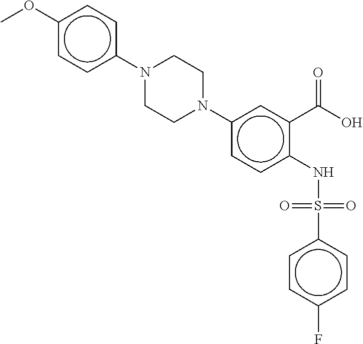 |
| | | | S(═O)(═O)(c1ccc(cc1)F)Nc2ccc(cc2C(O)═O)N(CC3)CCN3c4ccc(cc [symbol] |
| ChemDiv Targeted Diversity Library | Chem Div | F685- 0437 | 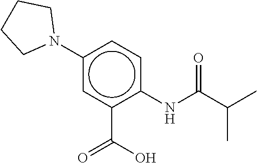 |
| | | | c1(C(O)═O)cc(ccc1NC(C(C)C)═O)N2CCCC2 |
| ChemDiv Targeted Diversity Library | Chem Div | F685- 1588 | 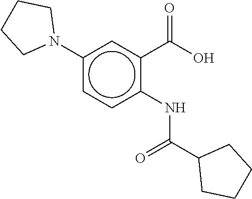 |
| | | | c1(C(O)═O)cc(ccc1NC(C2CCCC2)═O)N3CCCC3 |
| ChemDiv Targeted Diversity Library | Chem Div | F688- 0005 | 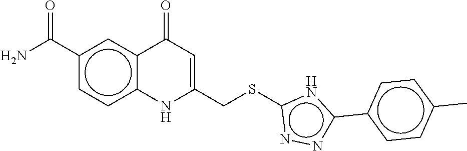 |
| | | | [nH]1c(SCC(═CC2═O)Nc(c23)ccc(c3)C(N)═O)nnc1c4ccc(cc4) [symbol] |
| ChemDiv Targeted Diversity Library | Chem Div | F727- 1225 | 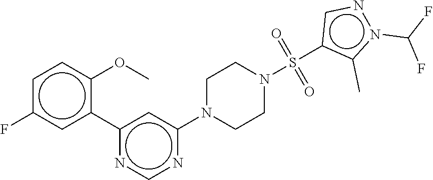 |
| | | | S(═O)(═O)(N(CC1)CCN1c2ncnc(c2)c3cc(F)ccc3OC)c(cnn4C(F)F) [symbol] |
| ChemDiv Targeted Diversity Library | Chem Div | F727- 1233 | 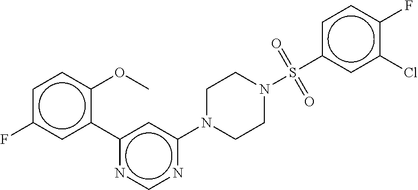 |
| | | | S(═O)(═O)c1ccc(c(Cl)c1)F)N(CC2)CCN2c3ncnc(c3)c4cc(F)occc4 [symbol] |
| ChemDiv Targeted Diversity Library | Chem Div | F726- 1263 | 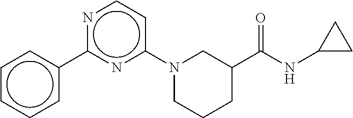 |
| | | | n1c(c2ccccc2)nccc1N(CCCC3C(═O)NC4CC4)C3 |
| ChemDiv Targeted Diversity Library | Chem Div | F781- 0170 | 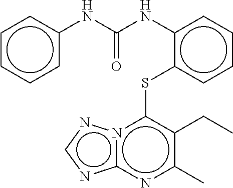 |
| | | | n1(ncnc2)c2nc(c(CC)c1Sc3ccccc3NC(═O)Nc4ccccc4)C |
| ChemDiv Targeted Diversity Library | Chem Div | F792- 1521 | 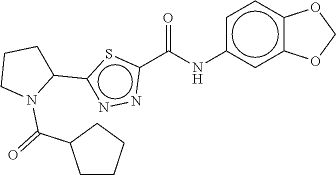 |
| | | | c(C(═O)Nc(ccc(c12)OCO1)c2)(nnc3C4CCCn4C(C5CCCCS)═O)s [symbol] |
| ChemDiv Targeted Diversity Library | Chem Div | F781- 0023 | 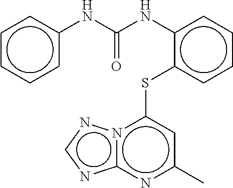 |
| | | | n1(ncn2)c2nc(C)cc1Sc3ccccc3NC(═O)Nc4ccccc4 |
| ChemDiv Targeted Diversity Library | Chem Div | F781- 0201 | 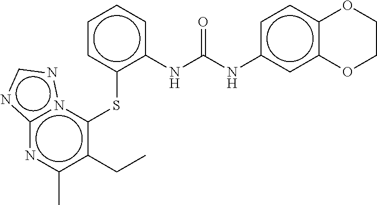 |
| | | | n1(ncn2)c2nc(c(CC)c1Sc3ccccc3NC(═O)Nc(ccc(c45)OCCO4)c5 [symbol] |
| ChemDiv Targeted Diversity Library | Chem Div | F793- 0010 | 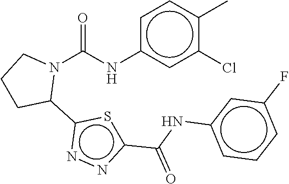 |
| | | | c(C(═O)Nc1cccc(F)c1)(nnc2C3CCCN3C(═O)Nc(ccc(c4Cl)c)c4)s [symbol] |
| ChemDiv Targeted Diversity Library | Chem Div | F793- 0015 | 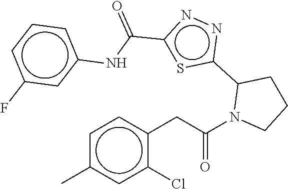 |
| | | | c(C(═O)Nc1cccc(F)c1)(nnc2C3CCCN3C(═O)Nc4ccc(cc4Cl)C)s [symbol] |
| ChemDiv Targeted Diversity Library | Chem Div | F793- 0016 | 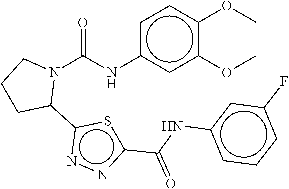 |
| | | | c(C(═O)Nc1cccc(F)c1)(nnc2C3CCCN3C(═O)Nc4ccc(c(OC)c4)OC [symbol] |
| ChemDiv Targeted Diversity Library | Chem Div | F781- 0032 | 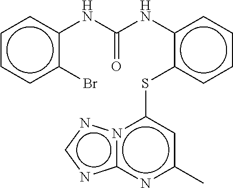 |
| | | | n1(ncn2)c2nc(C)cc1Sc3ccccc3NC(═O)Nc4ccccc4Br |
| ChemDiv Targeted Diversity Library | Chem Div | F781- 0523 | 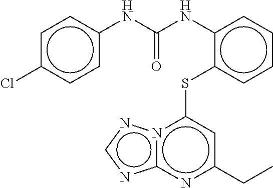 |
| | | | n1(ncn2)c2nc(CC)cc1Sc3ccccc3NC(═O)Nc(cc4)ccc4Cl |
| ChemDiv Targeted Diversity Library | Chem Div | F792- 0003 | 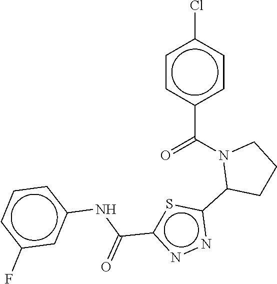 |
| | | | c(C(═O)Nc1cccc(F)c1)(nnc2C3CCCn3C(═O)c4ccc(cc4)Cl)s2 |
| ChemDiv Targeted Diversity Library | Chem Div | F798- 0626 | 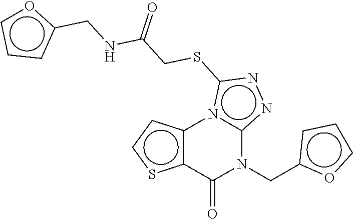 |
| | | | c12n(c(nn1)SCC(═O)NCc3ccco3)c(c4C(═O)N2Cc5ccco5)ccs4 [symbol] |
| ChemDiv Targeted Diversity Library | Chem Div | F835- 0569 | 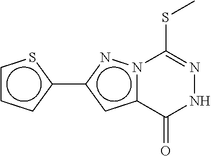 |
| | | | n12c(C(NN═C1SC)═O)cc(c3ccc3)n2 |
| ChemDiv Targeted Diversity Library | Chem Div | F818- 0094 | 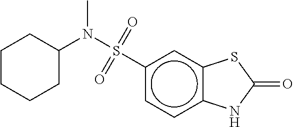 |
| | | | S(═O)(═O)(c(cc(c1S2)NC2═O)c1)N(C)C3CCCCC3 |
| ChemDiv Targeted Diversity Library | Chem Div | F835- 0135 | 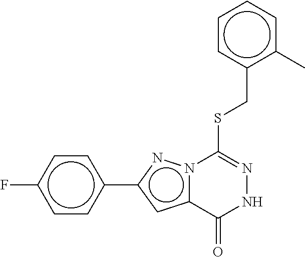 |
| | | | n12c(C(NN═C1SCc3ccccc3C)═O)cc(c4ccc(cc4)F)n2 |
| ChemDiv Targeted Diversity Library | Chem Div | F854- 0008 | 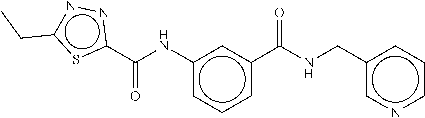 |
| | | | c1(C(═O)Nc(cccc2C(═O)NCc(cccn3)c3)c2)sc(nn1)CC |
| ChemDiv Targeted Diversity Library | Chem Div | F869- 1268 | 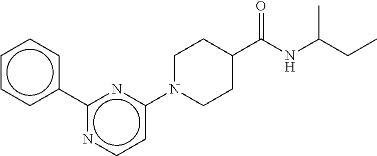 |
| | | | n1c(c2ccccc2)nccc1N(CC3)CCC3C(═O)NC(C)CC |
| ChemDiv Targeted Diversity Library | Chem Div | F854- 0333 | 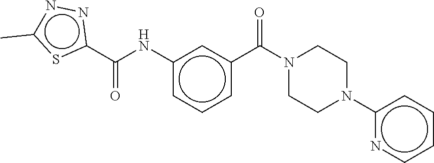 |
| | | | c1(C(═O)Nc(cccc2C(═O)N(CC3)CCN3c4ccccn4)c2)sc(nn1)C |
| ChemDiv Targeted Diveristy Library | Chem Div | F912- 0858 | 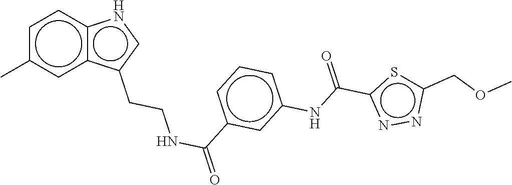 |
| | | | c1(C(═O)Nc(cccc2C(NCCc(c3)c4c([nH]3)ccc(C)c4)═O)c2)sc(nn1) [symbol] |
| ChemDiv Targeted Diversity Library | Chem Div | F912- 0859 | 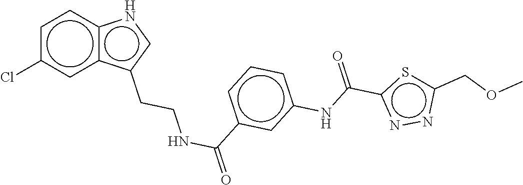 |
| | | | c1(C(═O)Nc(cccc2C(NCCc(c3)c4c([nH]3)ccc(Cl)c4)═O)c2)sc(nn1)COC |
| ChemDiv Targeted Diversity Library | Chem Div | G199- 0398 | 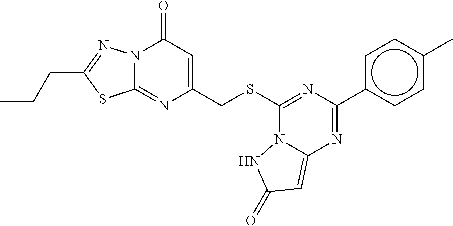 |
| | | | N1(N═C(S2)CCC)C2═NC(CSC3═NC(c4ccc(cc4)C)═NC(═CC(═O)N5)N35)═CC1═ [symbol] |
| ChemDiv Targeted Diversity Library | Chem Div | G199- 0400 | 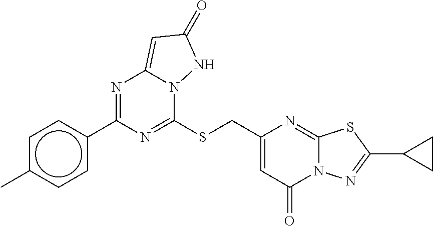 |
| | | | N12C(═NC(CSC3═NC(c4ccc(cc4)C)═NC(═CC(═O)N5)N35)═CC1═O)SC(C6CC6) [symbol] |
| ChemDiv Targeted Diversity Library | Chem Div | G189- 2182 | 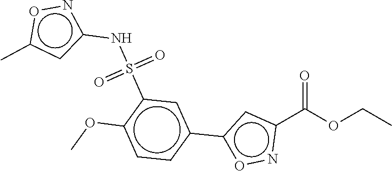 |
| | | | S(═O)(═O)Nc1cc(on1)C)c2c(OC)ccc(c2)c(onc3C(OCC)═O)c3 [symbol] |
| ChemDiv Targeted Diversity Library | CHem Div | G199- 0048 | 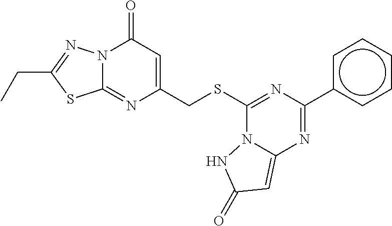 |
| | | | N1(N═C(S2)CC)C2═NC(CSC3═NC(c4ccccc4)═NC(═CC(═O)N5)N35)═ [symbol] |
| ChemDiv Targeted Diversity Library | Chem Div | G199- 2057 | 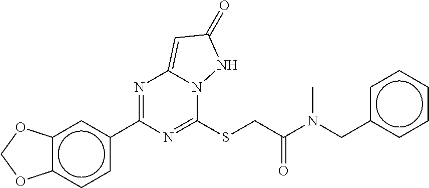 |
| | | | N12C(═CC(═O)N1)N═C(c(ccc(c34)OCO3)c4)N═C2SCC(═O)N(C)Cc5 [symbol] |
| ChemDiv Targeted Diversity Library | Chem Div | G226- 0500 | 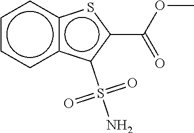 |
| | | | c1(C(OC)═O)sc(c2c1S(N)(═O)═O)cccc2 |
| ChemDiv Targeted Diversity Library | Chem Div | G747- 0002 | 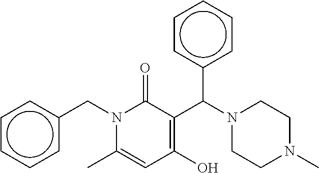 |
| | | | C1(C(c2ccccc2)N(CC3)CCN3C)═C(O)C═C(N(Cc4ccccc4)C1═O) [symbol] |
| ChemDiv Targeted Diversity Library | Chem Div | G786- 1562 | 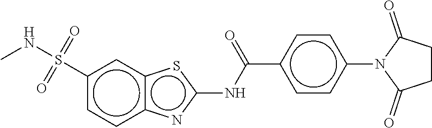 |
| | | | c1(sc(c(ccc2S(NC)(═O)═O)n1)c2)NC(═O)c(cc3)ccc3N4C(═O)CCC [symbol] |
| ChemDiv Targeted Diversity Library | Chem Div | G786- 0335 | 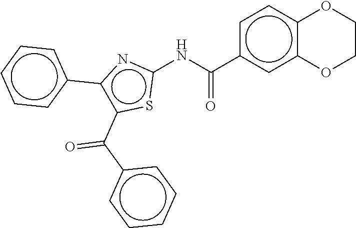 |
| | | | c(C(c1ccccc1)═O)(s2)c(c3ccccc3)nc2NC(═O)c(ccc(c45)OCCO4) |
| ChemDiv Targeted Diversity Library | Chem Div | G784- 0958 | 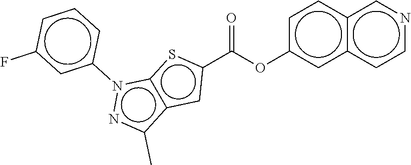 |
| | | | c12c(c(nn1c3cccc(F)c3)C)cc(C)═O)Oc(ccc(c4ccn5)c5)c4)s2 |
| ChemDiv Targeted Diversity Library | Chem Div | G784- 0099 | 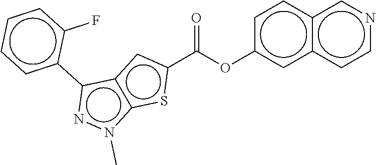 |
| | | | c1(cc(s2)C(═O)Oc(ccc(c3ccn4)c4)c3)c2n(nc1c5ccccc5F)C |
| ChemDiv Targeted Diversity Library | Chem Div | G786- 1547 | 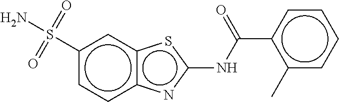 |
| | | | c1(sc(c(ccc2S(N)(═O)═)n1)c2)NC(═O)c3ccccc3C |
| ChemDiv Targeted Diversity Library | Chem Div | G786- 1264 | 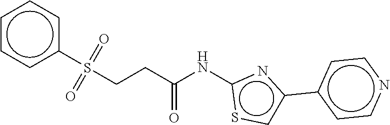 |
| | | | c1(scc(c(cc2)ccn2)n1)NC(═O)CCS(c3ccccc3)(═O)═O |
| ChemDiv Targeted Diversity Library | Chem Div | G821- 0669 | 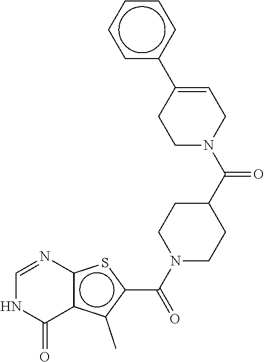 |
| | | | c1(s2)c(C(NC═N1)═O)c(c2C(═O)N(CC3)CCC3C(═O)N(CC4)CC═C4c5c [symbol] |
| ChemDiv Targeted Diversity Library | Chem Div | G843- 043 | 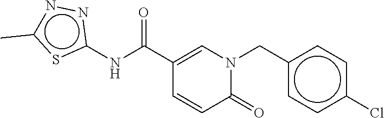 |
| | | | C(C═CC(N1Cc(cc2)ccc2Cl)═O)(═C1)C(═O)Nc3nnc(C)s3 |
| ChemDiv Targeted Diversity LIbrary | Chem Div | G843- 1071 | 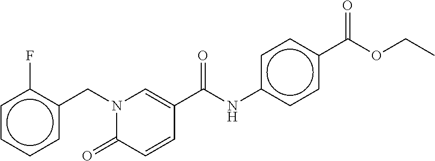 |
| | | | C(C═CC(N1Cc2ccccc2F)═O)(C(═O)Nc(cc3)ccc3C(OCC)═O)═C [symbol] |
| ChemDiv Targeted Diversity Library | Chem Div | G856- 6719 | 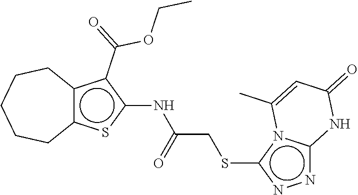 |
| | | | c1(nnc2SCC(═O)Nc3sc(c4c3C(OCC)═O)CCCCC4)n2C(═CC(═O)N [symbol] |
| ChemDiv Targeted Diversity Library | Chem Div | G856- 6165 | 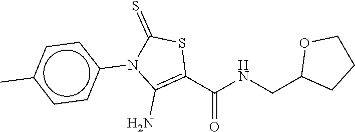 |
| | | | N1(c2ccc(cc2)C)C(═S)SC(C(═O)NCC3CCCO3)═C1N |
| ChemDiv Targeted Diversity Library | Chem Div | G857- 0928 | 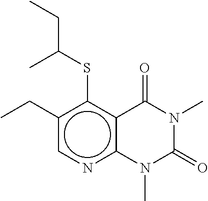 |
| | | | N(C)(C(═O)c1c(N2C)ncc(CC)c1SC(C)CC)C2═O |
| ChemDiv Targeted Diversity Library | Chem Div | G857- 2309 | 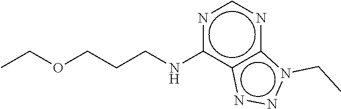 |
| | | | c12c(ncnc1NCCCOCC)n(nn2)CC |
| ChemDiv Targeted Diversity Library | Chem Div | G857- 2274 | 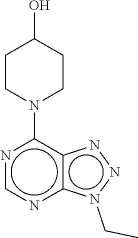 |
| | | | c12c(ncnc1N3CCC(CC3)O)n(nn2)CC |
| ChemDiv Targeted Diversity Library | Chem Div | G889- 0021 | 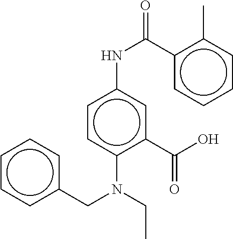 |
| | | | c1(cc(ccc1N(CC)Cc2ccccc2)NC(═O)c3ccccc3C)C(O)═O |
| ChemDiv Targeted Diversity Library | Chem Div | G890- 1803 | 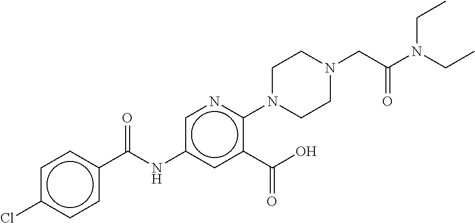 |
| | | | c1(cc(cnc1N(CC2)CCN2CC(═O)N(CC)CC)NC(═O)c3ccc(cc3)cl)c( [symbol] |
| ChemDiv Targeted Diversity Library | Chem Div | G889- 0745 | 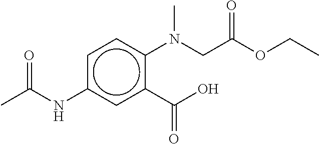 |
| | | | c1(cc(ccc1N(C)CC(OCC)═O)NC(C)═O)C(O)═O |
| ChemDiv Targeted Diveisty Library | Chem Div | G890- 0455 | 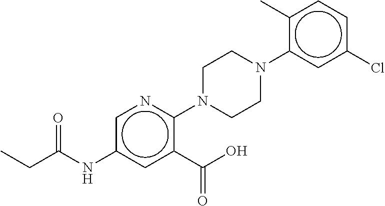 |
| | | | c1(cc(cnc1N(CC2)CCN2c3c(C)ccc(Cl)c3)NC(CC)═O)C(O)═O |
| ChemDiv Targeted Diversity Library | Chem Div | G889- 0171 | 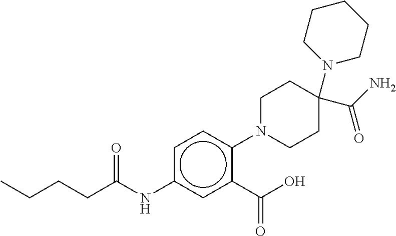 |
| | | | c1(cc(ccc1N(CC2)CCC2(C(N)═O)N3CCCCC3)NC(CCCC)═O)C(O)═ [symbol] |
| ChemDiv Targeted Diversity Library | Chem Div | G890- 0459 | 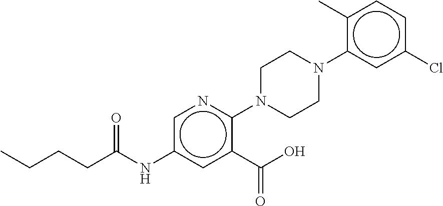 |
| | | | c1(cc(cnc1N(CC2)CCN2c3c(C)ccc(Cl)c3)NC(CCCC)═O)C(O)═ [symbol] |
| ChemDiv Targeted Diversity Library | Chem Div | G890- 0200 | 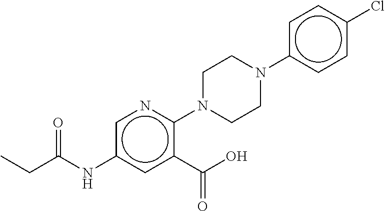 |
| | | | c1(cc(cnc1N(CC2)CCN2c3ccc(cc3)Cl)NC(CC)═O)C(O)═O |
| ChemDiv Targeted Diversity Library | Chem Div | G946- 0149 | 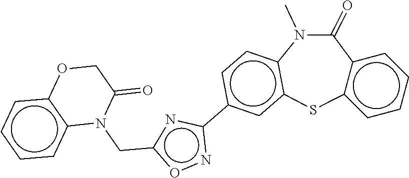 |
| | | | C1(═O)c2c(cccc2)Sc(cc3c4noc(CN5c6c(cccc6)OCC5═O)n4)c(cc3) [symbol] |
| ChemDiv Targeted Diversity Library | Chem Div | J015- 0261 | 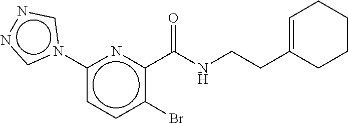 |
| | | | n1c(ccc(Br)c1C(NCCC2═CCCCC2)═O)n(cnn3)c3 |
| ChemDiv Targeted Diversity Library | Chem Div | J015- 0388 | 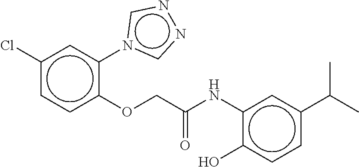 |
| | | | n(c1)(cnn1)c2c(ccc(Cl)c2)OCC(═O)Nc3c(O)CCC(C(C)C)c3 |
| ChemDiv Targeted Diversity Library | Chem Div | J021- 3314 | 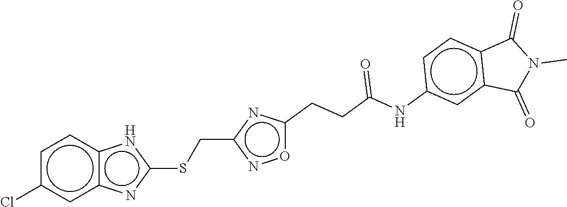 |
| | | | N1(C)C(═O)c2c(cc(cc2)NC(CC3onc(CSc4[nH]c(C5n4)ccc(Cl)c5)n3)═ [symbol] |
| ChemDiv Targeted Diversity Library | CHem Div | J021- 3320 | 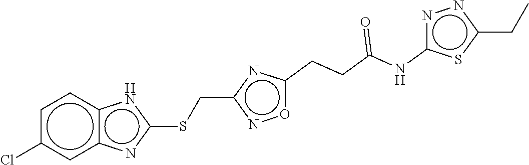 |
| | | | c1(NC(CCc2onc(CSc3[nH]c(c4n3)ccc(Cl)c4)nZ)═O)nnc(CC)s1 [symbol] |
| ChemDiv Targeted Diversity Library | Chem Div | J035- 0001 | 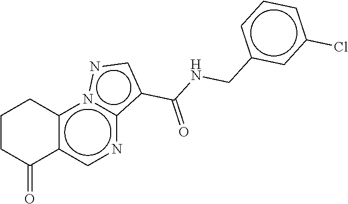 |
| | | | c1(nc2)n(ncc1C(═O)NCc3cccc(Cl)c3)c(c24)CCCC4═O |
| ChemDiv Targeted Diversity Library | Chem Div | J065- 2258 | 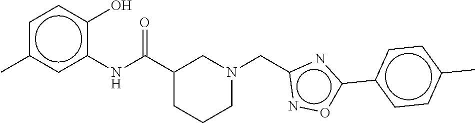 |
| | | | n1c(c2ccc(cc2)C)onc1CN(CCCC3C(═O)Nc4cc(C)ccc4O)C3 |
| ChemDiv Targeted Diversity Library | Chem Div | J094- 187 | 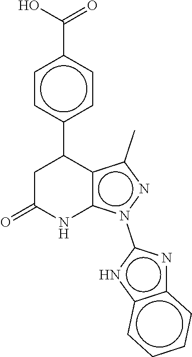 |
| | | | n1(nc(c(C(CC(═O)N2)c(cc3)ccc3C(O)═O)c12)C)c4[nH](c(c5n4)cc [symbol] |
| ChemDiv Targeted Diversity Library | Chem Div | K261- 1443 | 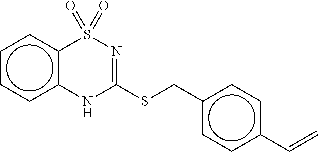 |
| | | | S1(═O)(═O)c2c(cccc2)NC(SCc(cc3)ccc3C═C)═N1 |
| ChemDiv Targeted Diversity LIbary | Chem Div | K261- 1972 | 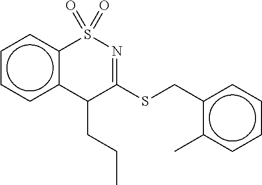 |
| | | | S1(═O)(═O)c2c(cccc2)N(C(SCc3ccccc3C)═N1)CCC |
| ChemDiv Targeted Diversity Library | Chem Div | K784- 4049 | 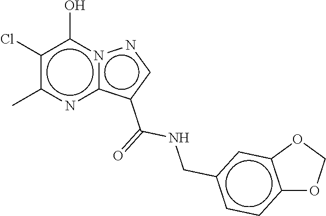 |
| | | | c1(nc(C)c(c2O)Cl)n2ncc1C(═O)NCc(ccc(c34)OCO3)c4 |
| ChemDiv Targeted Diversity Library | Chem Div | L062- 0524 | 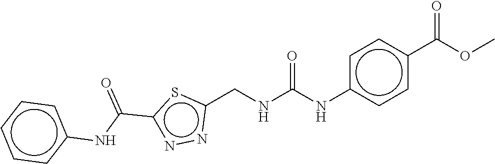 |
| | | | c1(C(═O)Nc2ccccc2)sc(nn1)CNC(═O)Nc(cc3)ccc3C(OC)═O |

[symbol]

indicates data missing or illegible when filed

TABLE 4B
SaCas9 Weaker Potential Hits

| | Cpd | | Normalized | Normalized | Auto | Auto | 3 | 5 |
|---|---|---|---|---|---|---|---|---|
| | Vendor | | Zscore | Zscore | Zscore | Zscore | sigma | sigma |
| Cpd Lib | ID | Compound SMILE | Rep 1 | Rep 2 | Rep 1 | Rep 2 | Hit | Hit |
| ChemDiv | F293-0815 | S(═O)(═O)(c1ccc(c(F)c1)F)Nc(cnc(c2 [symbol] ═O)N(C)C)c2 | 0.208672 | 0.158988 | 1.676174 | 1.234836 | 1 | |
| Trgtd Div | | | | | | | | |
| Lib | | | | | | | | |
| 3641 E19 | | | 0.890798 | 0.909066 | 0.651931 | 0.314075 | 1 | 1 |
| 3641 O17 | | | 0.85453 | 0.838722 | 1.033104 | 0.2483 | 1 | 1 |
| 3641 N09 | | | 0.755481 | 0.649905 | −0.1071 | −0.91262 | 1 | 1 |
| Enamine 1 | T0501-2049 | COc1ccc(cc1)c1nc(N)s[n+]1c1ccccc1 | 0.605295 | 0.603252 | 0.56128 | 0.769096 | 1 | 1 |
| 3640 M06 | | | 0.599047 | 0.71145 | −1.05206 | −0.90028 | 1 | 1 |
| Enamine 1 | T0501-2919 | O═C(NCc1ccco1)C1C(═C)C1C(═O)NCcco1 | 0.506972 | 0.589205 | 0.741084 | 0.471701 | 1 | 1 |
| ChemDiv | C679-2752 | N1(N═C(S2)C)C2═NC(COc(ccc(c3C)C) [symbol] 3)═CC1═O | 0.503382 | 0.529206 | 0.935063 | −0.94273 | 1 | 1 |
| Trgtd Div | | | | | | | | |
| Lib | | | | | | | | |
| Enamine 1 | T0513-3165 | Cc1nn(c(Nc2ccccc2)c1)c1ccccc1C(═O)O | 0.493308 | 0.451675 | 0.053934 | −1.23341 | 1 | 1 |
| ChemDiv | E722-2652 | c12c(CSC(C(NCCCN(CC3)CCC3N4CCCCC4)═O)═C1)c5c(CCCC5)s2 | 0.460581 | 0.319174 | 1.567355 | 0.380657 | 1 | 1 |
| Trgtd Div | | | | | | | | |
| Lib | | | | | | | | |
| ChemDiv | F293-0458 | S(═O)(═O)(Nc(cnc(c1C(O)═O)N2CCC [symbol] )c1)c3c(C)cc(c(C)c3)C | 0.450914 | 0.429034 | 1.794463 | 2.846117 | 1 | 1 |
| Trgtd Div | | | | | | | | |
| Lib | | | | | | | | |
| ChemDiv | D715-0012 | c12c(ccc(O)c1O)C3═C(C(═O)O2)CCC [symbol] | 0.440225 | 0.58225 | −0.80663 | −0.80678 | 1 | 1 |
| Trgtd Div | | | | | | | | |
| Lib | | | | | | | | |
| ChemDiv | D656-0040 | c1(C(═O)N([H])c2ccc(c3c2cccn3)OCC)c(C)c4c(cc(cc4)C)o1 | 0.438861 | 0.358064 | −0.41304 | −1.28689 | 1 | 1 |
| Trgtd Div | | | | | | | | |
| Lib | | | | | | | | |
| Enamine 1 | T0513-4457 | O═C1CC(NC(═O)c2ccc(Cl)cc2)C(═O [symbol] | 0.436118 | 0.413235 | 0.256982 | 0.399771 | 1 | 1 |
| Enamine 1 | T0509-3636 | O═C(NCCc1ccc(O)c(O)c1)CSc1ccc2c [symbol] c2c1 | 0.435101 | 0.356192 | 0.88608 | 0.304827 | 1 | 1 |
| ChemDiv | D733-0293 | c1(C2c3ccc(c(O)c3)O)c(onc1C(C)(C) [symbol] (C)(C)CC4═O)═C24 | 0.419075 | 0.363361 | −1.04347 | 2.014824 | 1 | 1 |
| Trgtd Div | | | | | | | | |
| Lib | | | | | | | | |
| ChemDiv | C202-1892 | c12c(n[nH]c1c3ccc(cc3)OC)nc(c4ccc [symbol] O)c4)O)cc2C(O)═O | 0.408444 | 0.404269 | −1.82007 | −0.39259 | 1 | 1 |
| Trgtd Div | | | | | | | | |
| Lib | | | | | | | | |
| ChemDiv | F293-0898 | c1(cc(cnc1N2CCC(CC2)CCN(CC3)CCO3)NS(C)(═O)═O)C(O)═O | 0.385761 | 0.277043 | 0.362767 | 0.237376 | 1 | 1 |
| Trgtd Div | | | | | | | | |
| Lib | | | | | | | | |
| ChemDiv | F128-0041 | C(C(═O)N1CCCC1)(SC(N2Cc3ccccc3)C2N | 0.357588 | 0.384518 | 1.187536 | −1.11462 | 1 | 1 |
| Trgtd Div | | | | | | | | |
| Lib | | | | | | | | |
| ChemDiv | D727-0525 | n1(nc(s2)COc(cc3)ccc3F)c2nnc1c4c [symbol] n4 | 0.356625 | 0.342587 | 0.939285 | 1.06575 | 1 | 1 |
| Trgtd Div | | | | | | | | |
| Lib | | | | | | | | |
| ChemDiv | D361-0120 | c12c(c(n[nH]1)C)C(CC(═O)N2)c3ccc(c(O)c3)O | 0.353299 | 0.282105 | −0.20298 | −0.05734 | 1 | 1 |
| Trgtd Div | | | | | | | | |
| Lib | | | | | | | | |
| ChemDiv | C795-1664 | c1(n2cccc2CNCc3cccc(OC)c3)sc(c(C)c1C(O)═O)C | 0.34936 | 0.328477 | 0.429043 | 0.830163 | 1 | 1 |
| Trgtd Div | | | | | | | | |
| Lib | | | | | | | | |
| ChemDiv | D727-0522 | n1(nc(s2)COc(cc3)ccc3Br)c2nnc1c4ccccn4 | 0.347052 | 0.377602 | 1.020891 | 0.529695 | 1 | 1 |
| Trgtd Div | | | | | | | | |
| Lib | | | | | | | | |
| ChemDiv | C742-0431 | n12c(ccc(SCC(═O)Nc3nnc(C)s3)n1)nnc2c4cccc(F)c4 | 0.341988 | 0.31069 | −0.06844 | 0.20735 | 1 | 1 |
| Trgtd Div | | | | | | | | |
| Lib | | | | | | | | |
| ChemDiv | F293-0589 | S(═O)(═O)(Nc(cnc(c1C(O)═O)N(CC(C)C)CC(C)C)c1)c2c(C)cc(c(C)c2)C | 0.335011 | 0.292893 | 1.65578 | 0.299717 | 1 | 1 |
| Trgtd Div | | | | | | | | |
| Lib | | | | | | | | |
| | | | 0.332112 | 0.378491 | 1.228662 | −0.04769 | 1 | 1 |
| ChemDiv | F293-0441 | S(═O)(═O)(c1ccc(c2c1)CCCC2)Nc(cnc(c3C(O)═O)N4CCCC4)c3 | 0.330492 | 0.304403 | 1.398809 | 1.863044 | 1 | 1 |
| Trgtd Div | | | | | | | | |
| Lib | | | | | | | | |
| | | | 0.321378 | 0.24242 | 0.11585 | 0.702253 | 1 | |
| ChemDiv | F128-0076 | N1(c2ccc(cc2)Cl)C(═S)SC(C(═O)NCC3CCCO3)═C1N | 0.316765 | 0.206445 | −0.62852 | −1.45073 | 1 | 1 |
| Trgtd Div | | | | | | | | |
| Lib | | | | | | | | |
| Enamine 1 | T0512-2617 | O═C(CSc1nnc2ccccn12)N1CCc2ccccc2C1 | 0.306572 | 0.3358 | −1.3165 | 0.880965 | 1 | 1 |
| ChemDiv | F293-0006 | c1(cc(cnc1N(CC)Cc2ccccc2)NS(CC)(═O)═O)C(O)═O | 0.30224 | 0.295655 | 1.341704 | 0.92313 | 1 | 1 |
| Trgtd Div | | | | | | | | |
| Lib | | | | | | | | |
| ChemDiv | F293-0515 | S(═O)(═O)(Nc(cnc(c1C(O)═O)N(CC)CCN(C)C)c1)c2c(C)cc(c(C)c2)C | 0.29525 | 0.305619 | 2.128933 | 2.544002 | 1 | 1 |
| Trgtd Div | | | | | | | | |
| Lib | | | | | | | | |
| Enamine 1 | T0507-0044 | O═C(CSc1cc(C)ccc1C)NC(C)C(N1CC [symbol] CC1)c1ccccc1 | 0.292291 | 0.241726 | 1.661634 | −1.14008 | 1 | 1 |
| ChemDiv | D727-0351 | n1(nc(s2)COc3ccccc3C)c2nnc1C(C)(C)C | 0.289645 | 0.322712 | −0.02144 | −0.30417 | 1 | 1 |
| Trgtd Div | | | | | | | | |
| Lib | | | | | | | | |
| ChemDiv | F294-0983 | S(═O)(═O)(N(C)C)Nc(ccc(c1C(O)═O)N2CCCC(CN3CCCC3)C2)c1 | 0.28943 | 0.257707 | −0.38775 | −0.58745 | 1 | 1 |
| Trgtd Div | | | | | | | | |
| Lib | | | | | | | | |
| ChemDiv | F293-0183 | c1(cc(cnc1N(CC2)CCC2(C(N)═O)N3CCCCC3)NS(C)(═O)═O)C(O)═O | 0.28787 | 0.328342 | 1.533413 | −0.41481 | 1 | 1 |
| Trgtd Div | | | | | | | | |
| Lib | | | | | | | | |
| ChemDiv | D727-0112 | n1(nc(s2)COc(cc3)ccc3C)c2nnc1c4ccoc4C | 0.286527 | 0.312744 | −0.15127 | −0.22972 | 1 | 1 |
| Trgtd Div | | | | | | | | |
| Lib | | | | | | | | |
| ChemDiv | F128-0042 | C(C(═O)N1CCCCC1)(SC(N2Cc3ccccc3 [symbol] )═S)═C2N | 0.280134 | 0.261196 | −0.34357 | 0.303757 | 1 | 1 |
| Trgtd Div | | | | | | | | |
| Lib | | | | | | | | |
| ChemDiv | F293-0563 | c1(cc(cnc1N(CC(C)C)CC(C)C)NS(CC)(═O)═O)C(O)═O | 0.279157 | 0.31213 | 1.978013 | 2.222705 | 1 | 1 |
| Trgtd Div | | | | | | | | |
| Lib | | | | | | | | |
| ChemDiv | D727-0114 | n1(nc(s2)COc(cc3)ccc3Br)c2nnc1c4ccoc4C | 0.276642 | 0.263465 | 1.154429 | −0.65657 | 1 | 1 |
| Trgtd Div | | | | | | | | |
| Lib | | | | | | | | |
| ChemDiv | E722-2588 | c12c(CSC(C(NCCc(cc3)ccc3S(N)(═O)═O)═O)═C1)c4c(CCCC4)s2 | 0.274584 | 0.287056 | 0.224705 | −0.34072 | 1 | 1 |
| Trgtd Div | | | | | | | | |
| Lib | | | | | | | | |
| ChemDiv | F086-0004 | C(═O)(Nc1cc(ccc1N(CC2)CCN2CCC)C(O)═O)c3ccccc3Cl | 0.27251 | 0.21084 | −1.48261 | 1.076006 | 1 | 1 |
| Trgtd Div | | | | | | | | |
| Lib | | | | | | | | |
| ChemDiv | F293-0004 | S(c1ccccc1)(═O)(═O)Nc(cnc(c2C(O)═O)N(CC)Cc3ccccc3)c2 | 0.270671 | 0.188386 | 1.668017 | 0.918334 | 1 | 1 |
| Trgtd Div | | | | | | | | |
| Lib | | | | | | | | |
| ChemDiv | D715-1040 | C1(═C2CCC1)c3c(OC2═O)cc(OCc([nH]nn4)n4)c(Cl)c3 | 0.269808 | 0.406376 | 0.032344 | 2.455613 | 1 | 1 |
| Trgtd Div | | | | | | | | |
| Lib | | | | | | | | |
| ChemDiv | D271-0002 | C12═C(C(CC(═O)N1)c3ccc(c(O)c3)O)C(NC(SCC)═N2)═O | 0.265507 | 0.320704 | −0.55921 | 0.10261 | 1 | 1 |
| Trgtd Div | | | | | | | | |
| Lib | | | | | | | | |
| ChemDiv | E234-0056 | C1(C)(Cn(c2c(c3)cccc2)c3C(═O)N1c4ccc(c5c4)OCCO5)C(═O)NC6CCCCC6 | 0.259211 | 0.159686 | 1.417629 | 1.056961 | 1 | |
| Trgtd Div | | | | | | | | |
| Lib | | | | | | | | |
| ChemDiv | F294-0003 | S(═O)(═O)(c1ccc(cc1)OC)Nc(ccc(c2C(O)═O)N(CC)Cc3ccccc3)c2 | 0.254708 | 0.272899 | 0.929735 | −1.42666 | 1 | 1 |
| Trgtd Div | | | | | | | | |
| Lib | | | | | | | | |
| ChemDiv | F293-0962 | S(═O)(═O)(N(C)C)Nc(cnc(c1C(O)═O)N2CCC(CC2)CCN3CCCCC3)c1 | 0.253343 | 0.27701 | −1.57471 | −1.35952 | 1 | 1 |
| Trgtd Div | | | | | | | | |
| Lib | | | | | | | | |
| ChemDiv | F293-0908 | n1c(C)c(c(C)n1C)CCCN(C)c2ncc(cc2C(O)═O)NS(CC)(═O)═O | 0.252725 | 0.277207 | −0.56722 | 0.414809 | 1 | 1 |
| Trgtd Div | | | | | | | | |
| Lib | | | | | | | | |
| ChemDiv | D588-0186 | c(o1)(NC(CC(c2ccccc2)CC3═O)═C3C [symbol] c5ccc(c(O)c5)O)c4c(n1)C | 0.252634 | 0.302083 | 0.285818 | −0.83366 | 1 | 1 |
| Trgtd Div | | | | | | | | |
| Lib | | | | | | | | |
| ChemDiv | C594-0003 | n1(C(c(cc2)ccn2)═O)nc(c(cc(ccc(OC)c3)c3n4)c14)N | 0.251498 | 0.197796 | −0.60222 | 0.66432 | 1 | |
| Trgtd Div | | | | | | | | |
| Lib | | | | | | | | |
| ChemDiv | F293-0010 | S(═O)(═O)(Nc(cnc(c1C(O)═O)N(CC)Cc2ccccc2)c1)c3cc(C)ccc3C | 0.250677 | 0.202823 | 2.149327 | 2.534411 | 1 | 1 |
| Trgtd Div | | | | | | | | |
| Lib | | | | | | | | |
| ChemDiv | F293-0426 | S(═O)(═O)(Nc(cnc(c1C(O)═O)N2CCC [symbol] c1)c3c(C)cc(cc3C)C | 0.246938 | 0.253103 | 0.811447 | 1.196472 | 1 | 1 |
| Trgtd Div | | | | | | | | |
| Lib | | | | | | | | |
| ChemDiv | F086-0028 | C(═O)(Nc1cc(ccc1N(CC2)CCN2CC(═O)NC(C)C)C(O)═O)c3ccccc3Cl | 0.246702 | 0.229465 | −0.07719 | 0.584737 | 1 | 1 |
| Trgtd Div | | | | | | | | |
| Lib | | | | | | | | |
| ChemDiv | F294-1002 | S(═O)(═O)(Nc(ccc(c1C(O)═O)N2CCC [symbol] CN3CCCC3)C2)c1)N(CC4)CCO4 | 0.245215 | 0.168097 | −0.79972 | −0.29972 | 1 | 1 |
| Trgtd Div | | | | | | | | |
| Lib | | | | | | | | |
| ChemDiv | F293-0814 | S(═O)(═O)(Nc(cnc(c1C(O)═O)N(C)C) [symbol] 1)c2c(F)ccc(F)c2 | 0.235819 | 0.237582 | 2.553139 | 2.946822 | 1 | 1 |
| Trgtd Div | | | | | | | | |
| Lib | | | | | | | | |
| ChemDiv | C660-1021 | c(cc(s1)C(O)═O)(c2c3ccc(cc3)Cl)c1n(n2)C | 0.23392 | 0.18851 | 1.316447 | 1.188311 | 1 | 1 |
| Trgtd Div | | | | | | | | |
| Lib | | | | | | | | |
| ChemDiv | F086-0619 | c1(cc(ccc1N(CC2)CCN2c3ccccn3)C(O [symbol] )═O)NC(═O)c4ccc(cc4)Br | 0.233541 | 0.145083 | 1.213349 | 0.122366 | 1 | 1 |
| Trgtd Div | | | | | | | | |
| Lib | | | | | | | | |
| ChemDiv | D715-0997 | n1n[nH]c(COc(ccc(c23)C═CC(═O)O2) [symbol] n1 | 0.233459 | 0.462813 | 1.322167 | 0.662888 | 1 | |
| Trgtd Div | | | | | | | | |
| Lib | | | | | | | | |
| ChemDiv | F293-0740 | S(═O)(═O)(Nc(cnc(c1C(O)═O)N(CC)C [symbol] c2c(C)cc(c(C)c2)C | 0.233348 | 0.211373 | 0.277111 | −0.13188 | 1 | 1 |
| Trgtd Div | | | | | | | | |
| Lib | | | | | | | | |
| ChemDiv | F294-0900 | S(═O)(═O)(N(C)C)Nc(ccc(c1C(O)═O)N [symbol] CCC(CC2)CCN(CC3)CCO3)c1 | 0.230715 | 0.229032 | −2.12128 | 0.251763 | 1 | 1 |
| Trgtd Div | | | | | | | | |
| Lib | | | | | | | | |
| ChemDiv | D727-0190 | n12c(nnc1CSc3ccccc3)sc(c4ccc(cc4)F)n2 | 0.227218 | 0.26845 | 0.84655 | −0.67643 | 1 | 1 |
| Trgtd Div | | | | | | | | |
| Lib | | | | | | | | |
| ChemDiv | E722-1395 | C1(NC(═O)C(SCc(c23)c(c(C)s2)C)═C3 [symbol] (C)N(N(c4ccccc4)C1═O)C | 0.225469 | 0.181763 | −0.03529 | 0.492009 | 1 | |
| Trgtd Div | | | | | | | | |
| Lib | | | | | | | | |
| ChemDiv | C301-8945 | n12c(c3c(cccc3)c(NCCCc4ccccc4)n1)nnn2 | 0.224756 | 0.162113 | 0.886786 | 0.99864 | 1 | |
| Trgtd Div | | | | | | | | |
| Lib | | | | | | | | |
| ChemDiv | F083-0067 | N1(c2c(ccc(Cl)c2)C(N3)═O)C3═C(SC1S)C(N([H])[H])═O | 0.224531 | 0.206759 | −0.52141 | 0.840004 | 1 | 1 |
| Trgtd Div | | | | | | | | |
| Lib | | | | | | | | |
| ChemDiv | F294-0002 | S(═O)(═O)(Nc(ccc(c1C(O)═O)N(CC)C [symbol] 2ccccc2)c1)c3c(C)cc(cc3C)C | 0.224115 | 0.204993 | −0.11854 | 0.011989 | 1 | 1 |
| Trgtd Div | | | | | | | | |
| Lib | | | | | | | | |
| ChemDiv | E218-0425 | n(C1)(c2c(c3)occ2)c3C(N(CCN( [symbol] | 0.223502 | 0.182065 | −0.46661 | 0.100663 | 1 | |
| Trgtd Div | | | | | | | | |
| Lib | | | | | | | | |
| | | | 0.219671 | 0.210743 | −0.09384 | −0.78765 | 1 | |
| ChemDiv | F294-0004 | S(c1ccccc1)(═O)(═O)Nc(ccc(c2C(O)═O)N(CC)Cc3ccccc3)c2 | 0.21862 | 0.186282 | −1.13827 | −1.13893 | 1 | |
| Trgtd Div | | | | | | | | |
| Lib | | | | | | | | |
| ChemDiv | D727-0624 | n12c(nnc1COc3ccccc3)sc(c4cc(on4)C)n2 | 0.215057 | 0.223246 | 1.239745 | 0.847357 | 1 | 1 |
| Trgtd Div | | | | | | | | |
| Lib | | | | | | | | |
| ChemDiv | D588-0188 | c(o1)(NC(CC(c2ccc(cc2)O)CC3═O)═C [symbol] C4c5ccc(c(O)c5)O)c4c(n1)C | 0.213256 | 0.219493 | 0.668527 | 0.237426 | 1 | 1 |
| Trgtd Div | | | | | | | | |
| Lib | | | | | | | | |
| ChemDiv | D588-0034 | c(o1)(NC(CC(c2ccc(c(OC)c2)OC)CC3═O)═C3C4c5ccc(c(O)c5)O)c4c(n1)C | 0.210603 | 0.261818 | 0.493812 | −1.18062 | 1 | 1 |
| Trgtd Div | | | | | | | | |
| Lib | | | | | | | | |
| ChemDiv | D686-0236 | c1(NC(═O)c2ccc(cc2)F)sc(nc1C(N)═O)Nc3cccc(C)c3C | 0.20773 | 0.493625 | 2.510059 | 1.918706 | 1 | |
| Trgtd Div | | | | | | | | |
| Lib | | | | | | | | |
| ChemDiv | C770-0245 | S(═O)(═O)(c1ccc(cc1)F)c2c3c(ccc(F)c3)ncc2C(c4ccccc4)═O | 0.206524 | 0.192914 | 0.166087 | 0.110143 | 1 | |
| Trgtd Div | | | | | | | | |
| Lib | | | | | | | | |
| ChemDiv | E722-2603 | C1(NC(═O)C(SCc(c23)c4c(CCCC4)s2)═C3)═C(C)N(N(c5ccccc5)C1═O)C | 0.20351 | 0.26197 | −0.30692 | 2.714222 | 1 | |
| Trgtd Div | | | | | | | | |
| Lib | | | | | | | | |
| ChemDiv | D727-0910 | n1(nc(s2)COc3ccccc3Cl)c2nnc1c4cc( [symbol] [nH]4)C | 0.202757 | 0.212615 | 0.871937 | 0.053273 | 1 | |
| Trgtd Div | | | | | | | | |
| Lib | | | | | | | | |
| ChemDiv | D727-0350 | n1(nc(s2)COc3ccccc3C)c2nnc1C( [symbol] | 0.202522 | 0.206538 | −0.2032 | 0.981371 | 1 | 1 |
| Trgtd Div | | | | | | | | |
| Lib | | | | | | | | |
| ChemDiv | F293-0002 | S(═O)(═O)(Nc(cnc(c1C(O)═O)N(CC)Cc2ccccc2)c1)c3c(C)cc(cc3C)C | 0.202267 | 0.229097 | 0.379083 | −0.09351 | 1 | |
| Trgtd Div | | | | | | | | |
| Lib | | | | | | | | |
| ChemDiv | D433-1057 | n1(nc(n2)CN(c3ccc(cc3)OCC)C(═O)Cc4ccccc4)c2NC(C)═CC1═O | 0.201987 | 0.160279 | 1.036045 | −0.87666 | 1 | 1 |
| Trgtd Div | | | | | | | | |
| Lib | | | | | | | | |
| ChemDiv | D271-0008 | C12═C(C(CC(═O)N1)c3ccc(c(O)c3)O)C(NC(SCc(cc4)ccc4C)═N2)═O | 0.197956 | 0.180966 | −0.47291 | −0.36373 | 1 | 1 |
| Trgtd Div | | | | | | | | |
| Lib | | | | | | | | |
| ChemDiv | F293-0436 | S(═O)(═O)(Nc(cnc(c1C(O)═O)N2CCC [symbol] 2)c1)c3c(OC)ccc(OC)c3 | 0.197748 | 0.22229 | −1.37077 | 1.263609 | 1 | |
| Trgtd Div | | | | | | | | |
| Lib | | | | | | | | |
| ChemDiv | D271-0007 | C12═C(C(CC(═O)N1)c3ccc(c(O)c3)O)NC(SCc4ccccc4)═N2)═O | 0.19422 | 0.179967 | −1.4647 | −0.19459 | 1 | 1 |
| Trgtd Div | | | | | | | | |
| Lib | | | | | | | | |
| ChemDiv | F293-0775 | S(═O)(═O)(Nc(cnc(c1C(O)═O)N(CC2) [symbol] 2)c1)c3c(F)ccc(F)c3 | 0.193944 | 0.164644 | −0.9384 | 0.683356 | 1 | |
| Trgtd Div | | | | | | | | |
| Lib | | | | | | | | |
| ChemDiv | F086-0033 | C(═O)(Nc1cc(ccc1N(CC2)CCN2CC(N [symbol] OC)═O)C(O)═O)c3ccccc3Cl | 0.19372 | 0.224568 | −0.0657 | −0.1907 | 1 | 1 |
| Trgtd Div | | | | | | | | |
| Lib | | | | | | | | |
| Enamine 1 | T0508-7813 | Oc1ccc(cc1)N1CCN(CC1)C(═O)CSc1 [symbol] 2ccccc2c1 | 0.19314 | 0.165036 | −2.32993 | −1.32572 | 1 | |
| ChemDiv | F290-0671 | S(═O)(═O)(N(C)C)Nc(ccc(c1C(O)═O)N(CC2)CCN2CC(═O)N3CCCC3)c1 | 0.192189 | 0.201705 | −0.45301 | −0.8512 | 1 | |
| Trgtd Div | | | | | | | | |
| Lib | | | | | | | | |
| ChemDiv | D727-0542 | n12c(nnc1c3ccccn3)sc(c(cc4)ccn4)n2 | 0.192138 | 0.229484 | −0.37755 | 1.249398 | 1 | 1 |
| Trgtd Div | | | | | | | | |
| Lib | | | | | | | | |
| ChemDiv | D727-0066 | n1(nc(s2)Cc3ccccc3)c2nnc1c(cccn4) [symbol] | 0.191733 | 0.223622 | 0.245631 | 1.30896 | 1 | 1 |
| Trgtd Div | | | | | | | | |
| Lib | | | | | | | | |
| ChemDiv | D271-0012 | C12═C(C(CC(═O)N1)c3ccc(c(O)c3)O)C(NC(SCc4cccc(Cl)c4)═N2)═O | 0.190785 | 0.189305 | 0.25483 | 0.764853 | 1 | 1 |
| Trgtd Div | | | | | | | | |
| Lib | | | | | | | | |
| ChemDiv | C742-0312 | n12c(ccc(SCC(═O)Nc3nnc(C)s3)n1)n [symbol] 2c4ccc(cc4)F | 0.189745 | 0.266536 | −0.0443 | −0.62675 | 1 | |
| Trgtd Div | | | | | | | | |
| Lib | | | | | | | | |
| ChemDiv | F293-0616 | S(═O)(═O)(Nc(cnc(c1C(O)═O)N(CC2) [symbol] CC2N3CCCCC3)c1)c4c(F)ccc(F)c4 | 0.18871 | 0.178784 | −0.18788 | −0.18463 | 1 | |
| Trgtd Div | | | | | | | | |
| Lib | | | | | | | | |
| ChemDiv | F128-0030 | N1(Cc2ccccc2)C(═S)SC(C(N)═O)═C1 [symbol] | 0.18695 | 0.143009 | −0.43714 | 0.192842 | 1 | |
| Trgtd Div | | | | | | | | |
| Lib | | | | | | | | |
| ChemDiv | F293-0529 | c1(cc(cnc1N(CCCC)CC)NS(C)(═O)═O)C(O)═O | 0.181525 | 0.189472 | 0.154743 | −0.23738 | 1 | |
| Trgtd Div | | | | | | | | |
| Lib | | | | | | | | |
| ChemDiv | D727-0113 | n1(nc(s2)COc(ccc(c3C)C)c3)c2nnc1c [symbol] ccoc4C | 0.180164 | 0.162651 | 1.744221 | −0.99905 | 1 | |
| Trgtd Div | | | | | | | | |
| Lib | | | | | | | | |
| ChemDiv | F293-0001 | S(═O)(═O)(c1ccc(cc1)F)Nc(cnc(c2C(O)═O)N(CC)Cc3ccccc3)c2 | 0.18003 | 0.178127 | 0.448424 | 1.330746 | 1 | |
| Trgtd Div | | | | | | | | |
| Lib | | | | | | | | |
| ChemDiv | D271-0011 | C12═C(C(CC(═O)N1)c3ccc(c(O)c3)O)C(NC(SCc(cc4)ccc4Cl)═N2)═O | 0.179677 | 0.206557 | −2.69993 | −0.04169 | 1 | 1 |
| Trgtd Div | | | | | | | | |
| Lib | | | | | | | | |
| ChemDiv | F293-0526 | c1(cc(cnc1N(CC [symbol] | 0.178957 | 0.185986 | 0.795131 | −0.08392 | 1 | |
| Trgtd Div | | | | | | | | |
| Lib | | | | | | | | |
| ChemDiv | F294-0012 | S(═O)(═O)(Nc(ccc(c1C(O)═O)N(CC)C [symbol] cc2)c1)c3c(OC)ccc(OC)c3 | 0.177201 | 0.143434 | −1.67668 | −1.26361 | 1 | |
| Trgtd Div | | | | | | | | |
| Lib | | | | | | | | |
| ChemDiv | F294-0009 | c1(cc(ccc1N(CC)Cc2ccccc2)NS(C)(═OC(O)═O | 0.176876 | 0.154976 | 0.011982 | 0.246967 | 1 | |
| Trgtd Div | | | | | | | | |
| Lib | | | | | | | | |
| ChemDiv | E135-0568 | S(═O)(═O)(N═C(c1ccc(cc1)F)C═C2C(═ [symbol] )Nc3sc(c4c3C(N)═O)CCCC4)N2C | 0.173054 | 0.145791 | 0.365257 | −0.5622 | 1 | |
| Trgtd Div | | | | | | | | |
| Lib | | | | | | | | |
| ChemDiv | C594-0010 | n1(C(═O)c2ccc(cc2)F)nc(c(cc(ccc(OC)c3)c3n4)c14)N | 0.169845 | 0.25666 | 0.861641 | −0.48139 | 1 | |
| Trgtd Div | | | | | | | | |
| Lib | | | | | | | | |
| ChemDiv | F294-0010 | S(═O)(═O)(Nc(ccc(c1C(O)═O)N(CC)C [symbol] 2ccccc2)c1)c3cc(C)ccc3C | 0.169203 | 0.184539 | −0.20012 | 1.493792 | 1 | |
| Trgtd Div | | | | | | | | |
| Lib | | | | | | | | |
| ChemDiv | F294-0006 | c1(cc(ccc1N(CC)Cc2ccccc2)NS(CC)(═ [symbol] ═O)C(O)═O | 0.166797 | 0.179113 | −0.28578 | −0.72652 | 1 | |
| Trgtd Div | | | | | | | | |
| Lib | | | | | | | | |
| ChemDiv | D727-0342 | n12c(SCC(c3ccc(cc3)Cl)═N1)nnc2CC [symbol] | 0.163263 | 0.17713 | −0.62236 | 1.035969 | 1 | |
| Trgtd Div | | | | | | | | |
| Lib | | | | | | | | |
| ChemDiv | F294-0183 | S(═O)(═O)(N(C)C)Nc(ccc(c1C(O)═O)N [symbol] )CCC2(C(N)═O)N3CCCCC3)c1 | 0.162441 | 0.244191 | −1.66037 | −0.58265 | 1 | |
| Trgtd Div | | | | | | | | |
| Lib | | | | | | | | |
| ChemDiv | F305-0007 | N(CCCC1C(═O)Nc2cccc(C)c2)(C1)c3 [symbol] nn3)c4ccccc4 | 0.161368 | 0.163559 | −0.42038 | −0.15106 | 1 | |
| Trgtd Div | | | | | | | | |
| Lib | | | | | | | | |
| ChemDiv | D359-0009 | c(c(c1ccccc1)n(c23)c4c(NC2c5ccc(c( [symbol] O)c5)O)cccc4)(C6═O)c3N(C(═O)N6C [symbol] C | 0.160128 | 0.104393 | −1.66787 | −0.19258 | 1 | 1 |
| Trgtd Div | | | | | | | | |
| Lib | | | | | | | | |
| ChemDiv | D278-0687 | n12c(cc(C)c(cccc3OC)c13)nnc2SCC(═ [symbol] c4sc(c(C)c4C(OCC)═O)C | 0.152597 | 0.149458 | 0.010103 | 0.483901 | 1 | 1 |
| Trgtd Div | | | | | | | | |
| Lib | | | | | | | | |
| ChemDiv | E613-0091 | c1(NC(COCc(c2)noc2c(ccc(c34)OCO3)c4)═O)sc(c5c1C(N)═O)CCCC5 | 0.151512 | 0.360398 | −0.46784 | −1.99792 | 1 | |
| Trgtd Div | | | | | | | | |
| Lib | | | | | | | | |
| ChemDiv | D727-0339 | n12c(SCC(N)═N1)nnc2Cn(c3c(n4)cc [symbol] c3)c4C | 0.147766 | 0.205065 | −1.12313 | −1.93932 | 1 | |
| Trgtd Div | | | | | | | | |
| Lib | | | | | | | | |
| Enamine 1 | T0510-3387 | Cc1ccc(C)n1CCN1CCN(CC1)S(═O)(═O)c1ccccc1 | 0.143587 | 0.173722 | 1.125804 | −0.93659 | 1 | |
| Enamine 1 | T0503-3218 | O═c1c2cccc3cccc(c23)n1S(═O)(═O)c1cccs1 | 0.142603 | 0.122974 | 1.378985 | 1.342862 | 1 | |
| ChemDiv | D727-0612 | n12c(nnc1C(C)C)sc(c3cc(on3)C)n2 | 0.136758 | 0.197228 | −0.25885 | −0.26942 | 1 | |
| Trgtd Div | | | | | | | | |
| Lib | | | | | | | | |
| ChemDiv | D226-0165 | c12c(C(NC(═O)N1C)═O)n(c(SCC(CO)O)n2)Cc3cccc(C)c3 | 0.122352 | 0.112536 | −0.34602 | −0.1307 | 1 | |
| Trgtd Div | | | | | | | | |
| Lib | | | | | | | | |

[symbol]

indicates data missing or illegible when filed

TABLE 4C
Total Hit Compounds for SaCas9 Inhibitors

| | Cmpd | Cmpd | |
|---|---|---|---|
| Compound Lib | Vendor | Vendor ID | Compound SMILE |
| NIH Clinical | Sequoia | SAM001246643 | N[C@@H](C(═O)N[C@H]1[C@H]2SCC(═C(N2C1═O)C(═O)O) |
| Collection 1 - | Research | | CSc3c[nH]nn3)c4ccc(O)cc4•CC(O)CO |
| 2014 | Products | | |
| | Ltd. | | |
| NIH Clinical | Sequoia | SAM001246816 | Nc1nc(cs1)C(═NOCC(═O)O)C(═O)N[C@H]2[C@H]3SCC |
| Collection 1 - | Research | | (═C(N3C2═O)C(═O)O)C═C•O |
| 2014 | Products | | |
| | Ltd. | | |
| NIH Clinical | Tocris | SAM001247071 | O═c1n([se]c2ccccc12)c3ccccc3 |
| Collection 1 - | Cookson | | |
| 2014 | Ltd. | | |
| NIH Clinical | Sequoia | SAM001246846 | CC(C)[C@H]1NC(═O)[C@@H](NC(═O)c2ccc(C)c3oc4c(C)c(═O) |
| Collection 1 - | Research | | c(N)c(C(═O)N[C@H]5[C@@H](C)OC(═O)[C@H](C(C)C)N(C)C |
| 2014 | Products | | (═O)CN(C)C(═O)[C@@H]6CCCN6C(═O)[C@H](NC5═O)C(C)C) |
| | Ltd. | | c4nc23)[C@@H](C)OC(═O)[C@H](C(C)C)N(C)C(═O)CN(C)C(═O) |
| | | | [C@@H]7CCCN7C1═O |
| Biomol 4 - FDA | BIOMOL | AC-748 | c(c1CCN2C)([C@@]2([H])Cc3c4c(O)c(O)cc3)c4ccc1 |
| Approved Drug | | | |
| Library | | | |
| Biomol 4 - FDA | BIOMOL | G-430 | c(c(S([O—])(═O)═O)cc(S([O—]) |
| Approved Drug | | | (═O)═O)c1)(c(NC(═O)c2ccc(C)c(NC(═O)c3cccc(NC(═O) |
| Library | | | Nc4cccc(C(═O)Nc5c(C)ccc(C(═O)Nc6ccc(S([O—])(═O)═O) |
| | | | c7c6c(S([O—])(═O)═O)cc(S([O—])(═O)═O)c7)c5) |
| | | | c4)c3)c2)ccc8S([O—])(═O)═O)c18 |
| Biomol 4 - FDA | BIOMOL | GR-305 | c(c(O)c(c1c2)c(O)c(C)c(O[C@@H]3O[C@H](C)[C@H](O)[C@H] |
| Approved Drug | | | (O[C@@H]4O[C@H](C)[C@@H](O)[C@H](O)C4)C3)c1)(C(═OH] |
| Library | | | 6O[C@H](C)[C@@H](O)[C@H](O[C@@H]7O[C@H](C)[C@ |
| | | | @H](O) |
| Biomol 4 - FDA | BIOMOL | DL-326 | c1(C(═O)O)cc(N)ccc1O |
| Approved Drug | | | |
| Library | | | |
| Biomol 4 - FDA | BIOMOL | DL-431 | c1(O)cc(C[C@H](N)C(═O)O)ccc1O |
| Approved Drug | | | |
| Library | | | |
| Enamine 1 | Enamine | T0501-0191 | ClCc1ccc2sc(C)nc2c1 |
| Enamine 1 | Enamine | T0501-2919 | O═C(NCc1ccco1)C1C(═C)C1C(═O)NCc1ccco1 |
| Enamine 1 | Enamine | T0501-2049 | COc1ccc(cc1)c1nc(N)s[n+]1c1ccccc1 |
| Enamine 1 | Enamine | T0502-5596 | CNc1nc(c2ccccc2)[n+](s1)c1ccccc1 |
| Enamine 1 | Enamine | T0502-0200 | Fc1ccc(cc1)c1nc2C(═O)c3ccccc3C(═O)c2o1 |
| Enamine 1 | Enamine | T0501-6231 | C1CCC(CC1)Nc1nc(c2ccccc2)[n+](s1)c1ccccc1 |
| Enamine 1 | Enamine | T0502-6692 | Oc1c(CC═C)cccc1O |
| Enamine 1 | Enamine | T0503-0513 | CNc1ccc(O)cc1 |
| Enamine 1 | Enamine | T0504-0611 | OCn1c(═S)sc2ccccc12 |
| Enamine 1 | Enamine | T0504-2608 | C═CC[n+]1c(═O)c2ccccc2n2CCCCCc12 |
| Enamine 1 | Enamine | T0503-8092 | O═C1C═CC(═O)C(═C1)Nc1ccccc1 |
| Enamine 1 | Enamine | T0504-2139 | OC(═O)\C═C/c1ccc(1)o1 |
| Enamine 1 | Enamine | T0504-4465 | Oc1ccc(CCNC(═O)C(F)(F)F)cc1O |
| Enamine 1 | Enamine | T0505-0429 | Brc1ccc(cc1)N/C═C1\Sc2ccccc2C/1═S |
| ChemDiv1 | ChemDiv | 0717-0920 | Nc1ccc(cc1)C(═O)Nc1ccc(cc1)c1sc2cc(ccc2n1)NC(═O)c1ccc(N) |
| (Combilab and | | | cc1 |
| International) | | | |
| ChemDiv1 | ChemDiv | 1254-0268 | CCCCCCC1C(═O)NC(═S)NC1═O |
| (Combilab and | | | |
| International) | | | |
| ChemDiv1 | ChemDiv | 1538-0029 | COc1ccc(C═C2C(═O)NC(═S)NC2═O)cc1OCc1ccc(Cl)cc1Cl |
| (Combilab and | | | |
| International) | | | |
| ChemDiv1 | ChemDiv | 1503-0673 | OC(═O)CCN1C(═S)S/C(═C\c2ccco2)/C1═O |
| (Combilab and | | | |
| International) | | | |
| ChemDiv1 | ChemDiv | 1616-0071 | Cc1cc(C)nc(n1)NS(═O)(═O)c1ccc(cc1)Nc1cc(Cl)c2nonc2c1[N+] |
| (Combilab and | | | (═O)[O—] |
| International) | | | |
| ChemDiv1 | ChemDiv | 2027-0268 | S═C1S/C(═C\c2cc(cc(Br)c2O)[N+](═O)[O—])/ |
| (Combilab and | | | C(═O)N1c1ccc(cc1)[N+](═O)[O—] |
| International) | | | |
| ChemDiv1 | ChemDiv | 1927-8049 | CCSc1nnc2c(n1)OC(Nc1ccccc21)c1cc2OCOc2cc1[N+](═O)[O—] |
| (Combilab and | | | |
| International) | | | |
| ChemDiv1 | ChemDiv | 1959-0155 | CCOC(═O)c1ccc2NC(c3ccc(F)cc3)C3CC═CC3c2c1 |
| (Combilab and | | | |
| International) | | | |
| ChemDiv1 | ChemDiv | 2040-0282 | CCCSc1nnc2c(n1)OC(Nc1ccccc21)c1cc2OCOc2cc1[N+](═O)[O—] |
| (Combilab and | | | |
| International) | | | |
| ChemDiv1 | ChemDiv | 2050-0166 | CCCCSc1nnc2c(n1)OC(Nc1ccccc21)c1cc2OCOc2cc1[N+](═O)[O—] |
| (Combilab and | | | |
| International) | | | |
| ChemDiv1 | ChemDiv | 2049-0152 | CSc1nnc2c(n1)OC(Nc1ccccc21)c1ccc(o1)[N+](═O)[O—] |
| (Combilab and | | | |
| International) | | | |
| Enamine 1 | Enamine | T0507-6337 | NC(═O)COC(═O)\C═C/c1ccc(I)o1 |
| Enamine 1 | Enamine | T0507-0044 | O═C(CSc1cc(C)ccc1C)NC(C)C(N1CCOCC1)c1ccccc1 |
| Enamine 1 | Enamine | T0509-3636 | O═C(NCCc1ccc(O)c(O)c1)CSc1ccc2ccccc2c1 |
| Enamine 1 | Enamine | T0510-9145 | COc1ccc(cc1)N\C(═[N+]\c1ccc(OC)cc1)\SC |
| Enamine 1 | Enamine | T0510-7926 | Clc1nc2sccn2c1SC#N |
| Enamine 1 | Enamine | T0510-8045 | Cc1ccc(cc1)C(═O)Sc1nnc2ccccn12 |
| Enamine 1 | Enamine | T0512-2617 | O═C(CSc1nnc2ccccn12)N1CCc2ccccc2C1 |
| Enamine 1 | Enamine | T0515-0121 | O═C1C═CC(═O)N1c1cccc(Cl)c1C |
| Enamine 1 | Enamine | T0512-4688 | FCc1sc2ccccc2[n+]1C |
| Enamine 1 | Enamine | T0514-5155 | O═C1C═CC(═O)N1CCc1ccccc1 |
| Enamine 1 | Enamine | T0515-0141 | O═C1C═CC(═O)N1c1ccccc1Br |
| Enamine 1 | Enamine | T0514-5122 | CCCCc1ccc(cc1)N1C(═O)C═CC1═O |
| Enamine 1 | Enamine | T0513-4457 | O═C1CC(NC(═O)c2ccc(Cl)cc2)C(═O)N1 |
| Enamine 1 | Enamine | T0513-3165 | Cc1nn(c(Nc2ccccc2)c1)c1ccccc1C(═O)O |
| Enamine 1 | Enamine | T0514-5125 | COc1ccc(cc1OC)N1C(═O)C═CC1═O |
| Enamine 1 | Enamine | T0514-5181 | O═C1C═CC(═O)N1c1ccccc1C(F)(F)F |
| Enamine 1 | Enamine | T0514-5182 | Clc1ccc(cc1)CN1C(═O)C═CC1═O |
| Enamine 1 | Enamine | T0514-2693 | N#Cc1c(NC(═O)COc2cccc(F)c2)sc(C)c1C |
| Enamine 1 | Enamine | T0514-5196 | O═C1C═CC(═O)N1c1ccc(F)c(Cl)c1 |
| ChemDiv | ChemDiv | 7695-0166 | c1(nc(C)c(c2n1)cccc2C)NC3═NCN(CCCN(CC4)CCO4)CN3 |
| Targeted | | | |
| Diversity Library | | | |
| ChemDiv | ChemDiv | C066-5201 | n1c2c(ccc(OC)c2Cl)cc(c1s3)cc3C(NCC)═O |
| Targeted | | | |
| Diversity Library | | | |
| ChemDiv | ChemDiv | C200-7260 | C1(═NNC2═S)N2c(c3C(═O)N1CCC)ccs3 |
| Targeted | | | |
| Diversity Library | | | |
| ChemDiv | ChemDiv | C200-7011 | C1(═NNC2═S)N2c(ccs3)c3C(═O)N1Cc4ccccc4OCC |
| Targeted | | | |
| Diversity Library | | | |
| ChemDiv | ChemDiv | C200-7168 | C1(═NNC2═S)N2c(cccc3)c3C(═O)N1Cc4ccccc4 |
| Targeted | | | |
| Diversity Library | | | |
| ChemDiv | ChemDiv | C200-7262 | C1(═NNC2═S)N2c(c3C(═O)N1Cc4ccccc4)ccs3 |
| Targeted | | | |
| Diversity Library | | | |
| ChemDiv | ChemDiv | C200-9572 | N1═C(C═CC(═S)N1)N2CCCCCC2 |
| Targeted | | | |
| Diversity Library | | | |
| ChemDiv | ChemDiv | C202-1858 | c12c(n[nH]c1c3ccc(cc3)C)nc(c4ccc(c(O)c4)O)cc2C(O)═O |
| Targeted | | | |
| Diversity Library | | | |
| ChemDiv | ChemDiv | C202-1892 | c12c(n[nH]c1c3ccc(cc3)OC)nc(c4ccc(c(O)c4)O)cc2C(O)═O |
| Targeted | | | |
| Diversity Library | | | |
| ChemDiv | ChemDiv | C301-7218 | N1(c2c(cccc2)N═C(C(OCC)═O)C1═O)C(═O)c3cccc(Cl)c3 |
| Targeted | | | |
| Diversity Library | | | |
| ChemDiv | ChemDiv | C301-5391 | N1(C(C)═O)C(═O)c2c(C(═O)N1c3ccccc3)cccc2 |
| Targeted | | | |
| Diversity Library | | | |
| ChemDiv | ChemDiv | C430-0373 | C1(═O)c2c(CN1C3CCCCC3)cccc2C(O)═O |
| Targeted | | | |
| Diversity Library | | | |
| ChemDiv | ChemDiv | C660-1021 | c(cc(s1)C(O)═O)(c2c3ccc(cc3)Cl)c1n(n2)C |
| Targeted | | | |
| Diversity Library | | | |
| ChemDiv | ChemDiv | C679-2752 | N1(N═C(S2)C)C2═NC(COc(ccc(c3C)C)c3)═CC1═O |
| Targeted | | | |
| Diversity Library | | | |
| ChemDiv | ChemDiv | C742-0431 | n12c(ccc(SCC(═O)Nc3nnc(C)s3)n1)nnc2c4cccc(F)c4 |
| Targeted | | | |
| Diversity Library | | | |
| ChemDiv | ChemDiv | C795-1664 | c1(n2cccc2CNCc3cccc(OC)c3)sc(c(C)c1C(O)═O)C |
| Targeted | | | |
| Diversity Library | | | |
| ChemDiv | ChemDiv | C798-1346 | n1(c2c(nc1C)cccn2)c3c(C)ccc(C(O)═O)c3 |
| Targeted | | | |
| Diversity Library | | | |
| ChemDiv | ChemDiv | D087-0518 | S(═O)(═O)(Nc1ccc(cc1C)Cl)C(CCS2(═O)═O)C2 |
| Targeted | | | |
| Diversity Library | | | |
| ChemDiv | ChemDiv | D058-0209 | n(c1c([nH]2)cccc1)(c(SCC)nn3)c23 |
| Targeted | | | |
| Diversity Library | | | |
| ChemDiv | ChemDiv | D097-0031 | S(═O)(═O)(c(cccc1)c1C(═C2C(c3ccccc3)═O)OC(═O)CN4C(═O) |
| Targeted | | | CCC4═O)N2CC |
| Diversity Library | | | |
| ChemDiv | ChemDiv | D243-0426 | N(C(C)C(═O)Nc(cc1)ccc1Br)(C2═O)N═Nc(sc(c3ccccc3)c4)c24 |
| Targeted | | | |
| Diversity Library | | | |
| ChemDiv | ChemDiv | D271-0003 | C12═C(C(CC(═O)N1)c3ccc(c(O)c3)O)C(NC(SCCC)═N2)═O |
| Targeted | | | |
| Diversity Library | | | |
| ChemDiv | ChemDiv | D271-0012 | C12═C(C(CC(═O)N1)c3ccc(c(O)c3)O)C(NC(SCc4cccc(Cl)c4)═N2)═O |
| Targeted | | | |
| Diversity Library | | | |
| ChemDiv | ChemDiv | D278-0689 | n12c(cc(C)c(cccc3C)c13)nnc2SCC(═O)Nc4sc(c(C)c4C(OC)═O) |
| Targeted | | | C(OC)═O |
| Diversity Library | | | |
| ChemDiv | ChemDiv | D271-0004 | C12═C(C(CC(═O)N1)c3ccc(c(O)c3)O)C(NC(SCC═C)═N2)═O |
| Targeted | | | |
| Diversity Library | | | |
| ChemDiv | ChemDiv | D271-0005 | C12═C(C(CC(═O)N1)c3ccc(c(O)c3)O)C(NC(SCC#C)═N2)═O |
| Targeted | | | |
| Diversity Library | | | |
| ChemDiv | ChemDiv | D274-0130 | c12c(cnn1c3ccc(cc3)C)C(NC═N2)═S |
| Targeted | | | |
| Diversity Library | | | |
| ChemDiv | ChemDiv | D271-0007 | C12═C(C(CC(═O)N1)c3ccc(c(O)c3)O)C(NC(SCc4ccccc4)═N2)═O |
| Targeted | | | |
| Diversity Library | | | |
| ChemDiv | ChemDiv | D271-0008 | C12═C(C(CC(═O)N1)c3ccc(c(O)c3)O)C(NC(SCc(cc4)ccc4C)═N2)═O |
| Targeted | | | |
| Diversity Library | | | |
| ChemDiv | ChemDiv | D271-0009 | C12═C(C(CC(═O)N1)c3ccc(c(O)c3)O)C(NC(SCc4cccc(C)c4)═N2)═O |
| Targeted | | | |
| Diversity Library | | | |
| ChemDiv | ChemDiv | D271-0010 | C12═C(C(CC(═O)N1)c3ccc(c(O)c3)O)C(NC(SCc4ccccc4C)═N2)═O |
| Targeted | | | |
| Diversity Library | | | |
| ChemDiv | ChemDiv | D278-0687 | n12c(cc(C)c(cccc3OC)c13)nnc2SCC(═O)Nc4sc(c(C)c4C(OCC)═O)C |
| Targeted | | | |
| Diversity Library | | | |
| ChemDiv | ChemDiv | D271-0002 | C12═C(C(CC(═O)N1)c3ccc(c(O)c3)O)C(NC(SCC)═N2)═O |
| Targeted | | | |
| Diversity Library | | | |
| ChemDiv | ChemDiv | D271-0011 | C12═C(C(CC(═O)N1)c3ccc(c(O)c3)O)C(NC(SCc(cc4)ccc4Cl)═N2)═O |
| Targeted | | | |
| Diversity Library | | | |
| ChemDiv | ChemDiv | D297-0031 | c12c(c(nc(C(CCCN3C(═O)Cc4ccccc4C)C3)n1)O)nnn2Cc(cc5)ccc5F |
| Targeted | | | |
| Diversity Library | | | |
| ChemDiv | ChemDiv | D361-0120 | c12c(c(n[nH]1)C)C(CC(═O)N2)c3ccc(c(O)c3)O |
| Targeted | | | |
| Diversity Library | | | |
| ChemDiv | ChemDiv | D359-0009 | c(c(c1ccccc1)n(c23)c4c(NC2c5ccc(c(O)c5)O)cccc4)(C6═O)c3N |
| Targeted | | | (C(═O)N6C)C |
| Diversity Library | | | |
| ChemDiv | ChemDiv | D390-0881 | c12c(scc1c3ccc(cc3)Cl)N═CN(Cc(ccc(c45)OCO4)c5)C2═O |
| Targeted | | | |
| Diversity Library | | | |
| ChemDiv | ChemDiv | D421-0876 | c12c(ccc(c1)C(═O)Nc(cc3)ccc3F)NC(═CC2═O)C |
| Targeted | | | |
| Diversity Library | | | |
| ChemDiv | ChemDiv | D433-1829 | n1(nc(n2)CNc3ccccc3F)c2NC(CCC4)═C4C1═O |
| Targeted | | | |
| Diversity Library | | | |
| ChemDiv | ChemDiv | D433-1057 | n1(nc(n2)CN(c3ccc(cc3)OCC)C(═O)COc4ccccc4)c2NC(C)═CC1═O |
| Targeted | | | |
| Diversity Library | | | |
| ChemDiv | ChemDiv | D588-0034 | c(o1)(NC(CC(c2ccc(c(OC)c2)OC)CC3═O)═C3C4c5ccc(c(O)c5)O) |
| Targeted | | | c4c(n1)C |
| Diversity Library | | | |
| ChemDiv | ChemDiv | D588-0186 | c(o1)(NC(CC(c2ccccc2)CC3═O)═C3C4c5ccc(c(O)c5)O)c4c(n1)C |
| Targeted | | | |
| Diversity Library | | | |
| ChemDiv | ChemDiv | D588-0188 | c(o1)(NC(CC(c2ccc(cc2)O)CC3═O)═C3C4c5ccc(c(O)c5)O)c4c |
| Targeted | | | (n1)C |
| Diversity Library | | | |
| ChemDiv | ChemDiv | D588-0191 | c(o1)(NC(CCCC2═O)═C2C3c4ccc(c(O)c4)O)c3c(n1)C |
| Targeted | | | |
| Diversity Library | | | |
| ChemDiv | ChemDiv | D656-0040 | c1(C(═O)N([H])c2ccc(c3c2cccn3)OCC)c(C)c4c(cc(cc4)C)o1 |
| Targeted | | | |
| Diversity Library | | | |
| ChemDiv | ChemDiv | D588-0192 | c(o1)(NC(CC(C)(C)CC2═O)═C2C3c4ccc(c(O)c4)O)c3c(n1)C |
| Targeted | | | |
| Diversity Library | | | |
| ChemDiv | ChemDiv | D656-0061 | C(C(═O)N([H])c1ccc(c2c1cccn2)OCC)(Oc(ccc(c3)CC)c3C4═O)═C4 |
| Targeted | | | |
| Diversity Library | | | |
| ChemDiv | ChemDiv | D715-2437 | C1(═C2CCCC1)c3c(OC2═O)cc(O)c(O)c3 |
| Targeted | | | |
| Diversity Library | | | |
| ChemDiv | ChemDiv | D715-0012 | c12c(ccc(O)c1O)C3═C(C(═O)O2)CCC3 |
| Targeted | | | |
| Diversity Library | | | |
| ChemDiv | ChemDiv | D715-2438 | c12c(ccc(O)c1O)C3═C(C(═O)O2)CCCC3 |
| Targeted | | | |
| Diversity Library | | | |
| ChemDiv | ChemDiv | D715-1040 | C1(═C2CCC1)c3c(OC2═O)cc(OCc([nH]nn4)n4)c(Cl)c3 |
| Targeted | | | |
| Diversity Library | | | |
| ChemDiv | ChemDiv | D727-0063 | n12c(nnc1c(cccn3)c3)sc(c4ccccc4OCC)n2 |
| Targeted | | | |
| Diversity Library | | | |
| ChemDiv | ChemDiv | D727-0112 | n1(nc(s2)COc(cc3)ccc3C)c2nnc1c4ccoc4C |
| Targeted | | | |
| Diversity Library | | | |
| ChemDiv | ChemDiv | D727-0181 | n12c(nnc1Cn(c3c(n4)cccc3)c4C)sc(c5ccccn5)n2 |
| Targeted | | | |
| Diversity Library | | | |
| ChemDiv | ChemDiv | D727-0051 | n12c(nnc1c(cccn3)c3)sc(c4cccc(Br)c4)n2 |
| Targeted | | | |
| Diversity Library | | | |
| ChemDiv | ChemDiv | D727-0535 | n12c(nnc1c3ccccn3)sc(c4cc(c5o4)cccc5)n2 |
| Targeted | | | |
| Diversity Library | | | |
| ChemDiv | ChemDiv | D727-0350 | n1(nc(s2)COc3ccccc3C)c2nnc1C(C)C |
| Targeted | | | |
| Diversity Library | | | |
| ChemDiv | ChemDiv | D727-0712 | n1(c(CCc(ccc(c2OC)OC)c2)nn3)c3sc(c(cc4)ccn4)n1 |
| Targeted | | | |
| Diversity Library | | | |
| ChemDiv | ChemDiv | D727-0489 | n12c(nnc1c3ccccn3)sc(c4cccc(Br)c4)n2 |
| Targeted | | | |
| Diversity Library | | | |
| ChemDiv | ChemDiv | D727-0772 | n1(n2)c(nnc1c3cccc(F)c3)sc2c4c5c([nH]c4)cccc5 |
| Targeted | | | |
| Diversity Library | | | |
| ChemDiv | ChemDiv | D727-0066 | n1(nc(s2)Cc3ccccc3)c2nnc1c(cccn4)c4 |
| Targeted | | | |
| Diversity Library | | | |
| ChemDiv | ChemDiv | D727-0182 | n12c(nnc1Cn(c3c(n4)cccc3)c4C)sc(c(cccn5)c5)n2 |
| Targeted | | | |
| Diversity Library | | | |
| ChemDiv | ChemDiv | D727-0536 | n12c(nnc1c3ccccn3)sc(c(ccc(c45)OCO4)c5)n2 |
| Targeted | | | |
| Diversity Library | | | |
| ChemDiv | ChemDiv | D727-0351 | n1(nc(s2)COc3ccccc3C)c2nnc1C(C)(C)C |
| Targeted | | | |
| Diversity Library | | | |
| ChemDiv | ChemDiv | D727-0624 | n12c(nnc1COc3ccccc3)sc(c4cc(on4)C)n2 |
| Targeted | | | |
| Diversity Library | | | |
| ChemDiv | ChemDiv | D727-0394 | n12c(nnc1CC(C)C)sc(c3cnccn3)n2 |
| Targeted | | | |
| Diversity Library | | | |
| ChemDiv | ChemDiv | D727-0713 | n1(c(CCc(c(C)nn2c3ccccc3)c2C)nn4)c4sc(c5ccccc5F)n1 |
| Targeted | | | |
| Diversity Library | | | |
| ChemDiv | ChemDiv | D727-0740 | n12c(nnc1c3ccc(c4n3)cccc4)sc(c5cccc(F)c5)n2 |
| Targeted | | | |
| Diversity Library | | | |
| ChemDiv | ChemDiv | D727-0754 | n12c(nnc1c3ccc(c4n3)cccc4)sc(c5cccc(N(C)C)c5)n2 |
| Targeted | | | |
| Diversity Library | | | |
| ChemDiv | ChemDiv | D727-0522 | n1(nc(s2)COc(cc3)ccc3Br)c2nnc1c4ccccn4 |
| Targeted | | | |
| Diversity Library | | | |
| ChemDiv | ChemDiv | D727-0114 | n1(nc(s2)COc(cc3)ccc3Br)c2nnc1c4ccoc4C |
| Targeted | | | |
| Diversity Library | | | |
| ChemDiv | ChemDiv | D727-0165 | n1(nc(s2)Cc(ccc(c3OC)OC)c3)c2nnc1Cc(cc4)ccc4OC |
| Targeted | | | |
| Diversity Library | | | |
| ChemDiv | ChemDiv | D727-0755 | n12c(nnc1c3ccc(c4n3)cccc4)sc(c(cc5)ccc5N(C)C)n2 |
| Targeted | | | |
| Diversity Library | | | |
| ChemDiv | ChemDiv | D727-0786 | n1(n2)c(nnc1c3ccoc3C)sc2c4c5c([nH]c4)cccc5 |
| Targeted | | | |
| Diversity Library | | | |
| ChemDiv | ChemDiv | D727-0123 | n12c(nnc1c3ccoc3C)sc(c4ccccn4)n2 |
| Targeted | | | |
| Diversity Library | | | |
| ChemDiv | ChemDiv | D727-0055 | n12c(nnc1c(cccn3)c3)sc(c4ccccc4F)n2 |
| Targeted | | | |
| Diversity Library | | | |
| ChemDiv | ChemDiv | D727-0717 | n1(c(CCc(c(C)nn2c3ccccc3)c2C)nn4)c4sc(c(cc5)ccc5N(C)C)n1 |
| Targeted | | | |
| Diversity Library | | | |
| ChemDiv | ChemDiv | D727-0742 | n12c(nnc1c3ccc(c4n3)cccc4)sc(c5ccc(cc5)OC)n2 |
| Targeted | | | |
| Diversity Library | | | |
| ChemDiv | ChemDiv | D727-0524 | n1(nc(s2)COc3ccccc3Cl)c2nnc1c4ccccn4 |
| Targeted | | | |
| Diversity Library | | | |
| ChemDiv | ChemDiv | D727-0069 | n1(nc(s2)Cc(ccc(c3OC)OC)c3)c2nnc1c(cccn4)c4 |
| Targeted | | | |
| Diversity Library | | | |
| ChemDiv | ChemDiv | D727-0088 | n1(nc(s2)Cc(ccc(c34)OCCO3)c4)c2nnc1c(cccn5)c5 |
| Targeted | | | |
| Diversity Library | | | |
| ChemDiv | ChemDiv | D727-0047 | n12c(nnc1c(cccn3)c3)sc(c4ccc(cc4)C)n2 |
| Targeted | | | |
| Diversity Library | | | |
| ChemDiv | ChemDiv | D727-0059 | n12c(nnc1c(cccn3)c3)sc(c4ccccc4OC)n2 |
| Targeted | | | |
| Diversity Library | | | |
| ChemDiv | ChemDiv | D727-0743 | n12c(nnc1c3ccc(c4n3)cccc4)sc(c5ccc(c(OC)c5)OC)n2 |
| Targeted | | | |
| Diversity Library | | | |
| ChemDiv | ChemDiv | D727-0525 | n1(nc(s2)COc(cc3)ccc3F)c2nnc1c4ccccn4 |
| Targeted | | | |
| Diversity Library | | | |
| ChemDiv | ChemDiv | D727-0089 | n12c(nnc1c(cccn3)c3)sc(c(cccn4)c4)n2 |
| Targeted | | | |
| Diversity Library | | | |
| ChemDiv | ChemDiv | D727-0159 | n12c(nnc1Cc(cc3)ccc3OC)sc(c4ccccc4OC)n2 |
| Targeted | | | |
| Diversity Library | | | |
| ChemDiv | ChemDiv | D727-0025 | n12c(nnc1c3ccc(c(OC)c3)OC)sc(c(cccn4)c4)n2 |
| Targeted | | | |
| Diversity Library | | | |
| ChemDiv | ChemDiv | D727-0542 | n12c(nnc1c3ccccn3)sc(c(cc4)ccn4)n2 |
| Targeted | | | |
| Diversity Library | | | |
| ChemDiv | ChemDiv | D727-0768 | n1(n2)c(nnc1CC(C)C)sc2c3c4c([nH]c3)cccc4 |
| Targeted | | | |
| Diversity Library | | | |
| ChemDiv | ChemDiv | D727-0072 | n1(nc(s2)CCCc3ccccc3)c2nnc1c(cccn4)c4 |
| Targeted | | | |
| Diversity Library | | | |
| ChemDiv | ChemDiv | D727-0090 | n12c(nnc1c(cccn3)c3)sc(c(cc4)ccn4)n2 |
| Targeted | | | |
| Diversity Library | | | |
| ChemDiv | ChemDiv | D727-0190 | n12c(nnc1CSc3ccccc3)sc(c4ccc(cc4)F)n2 |
| Targeted | | | |
| Diversity Library | | | |
| ChemDiv | ChemDiv | D727-0049 | n12c(nnc1c(cccn3)c3)sc(c(cc4)ccc4C(C)(C)C)n2 |
| Targeted | | | |
| Diversity Library | | | |
| ChemDiv | ChemDiv | D727-0619 | n12c(nnc1c3ccc(cc3)F)sc(c4cc(on4)C)n2 |
| Targeted | | | |
| Diversity Library | | | |
| ChemDiv | ChemDiv | D727-0404 | n1(nc(s2)CCCc3ccccc3)c2nnc1c4cccc(F)c4 |
| Targeted | | | |
| Diversity Library | | | |
| ChemDiv | ChemDiv | D727-0119 | n1(nc(s2)COc3cccc(OC)c3)c2nnc1c4ccoc4C |
| Targeted | | | |
| Diversity Library | | | |
| ChemDiv | ChemDiv | D727-0417 | n12c(nnc1c3cccc(F)c3)sc(c(cc4C)c5c(n4)cccc5)n2 |
| Targeted | | | |
| Diversity Library | | | |
| ChemDiv | ChemDiv | D727-0837 | n1(nc(s2)CCc(c(C)nn3c4ccccc4)c3C)c2nnc1c(cc5)ccn5 |
| Targeted | | | |
| Diversity Library | | | |
| ChemDiv | ChemDiv | D727-0852 | n12c(nnc1c3ccccc3Cl)sc(c4cc(n[nH]4)CC(C)C)n2 |
| Targeted | | | |
| Diversity Library | | | |
| ChemDiv | ChemDiv | D727-0884 | n12c(nnc1c3cnccn3)sc(c4ccc(c(Br)c4)F)n2 |
| Targeted | | | |
| Diversity Library | | | |
| ChemDiv | ChemDiv | D727-0838 | n1(nc(s2)CCc(c(C)nn3c4ccccc4)c3C)c2nnc1c5cnccn5 |
| Targeted | | | |
| Diversity Library | | | |
| ChemDiv | ChemDiv | D727-0853 | n12c(nnc1c3cccc(Cl)c3)sc(c4cc(n[nH]4)CC(C)C)n2 |
| Targeted | | | |
| Diversity Library | | | |
| ChemDiv | ChemDiv | D727-0796 | n12c(nnc1CC(C)C)sc(c3cc(c4[nH]3)cccc4)n2 |
| Targeted | | | |
| Diversity Library | | | |
| ChemDiv | ChemDiv | D727-0878 | n12c(nnc1c(cc3)ccn3)sc(c4cc(n[nH]4)CC(C)C)n2 |
| Targeted | | | |
| Diversity Library | | | |
| ChemDiv | ChemDiv | E143-0032 | c12n(c(c3C(═O)N1Cc4ccccc4)cccc3)c(nn2)c5ccc(cc5)NC(C)═O |
| Targeted | | | |
| Diversity Library | | | |
| ChemDiv | ChemDiv | D727-0892 | n12c(nnc1c3cccc(F)c3)sc(c4ccc(c5n4)cccc5)n2 |
| Targeted | | | |
| Diversity Library | | | |
| ChemDiv | ChemDiv | D727-0912 | n1(nc(s2)COc3ccccc3OC)c2nnc1c4cc(n[nH]4)C |
| Targeted | | | |
| Diversity Library | | | |
| ChemDiv | ChemDiv | D733-0293 | c1(C2c3ccc(c(O)c3)O)c(onc1C(C)(C)C)NC(CC(C)(C)CC4═O)═C24 |
| Targeted | | | |
| Diversity Library | | | |
| ChemDiv | ChemDiv | D727-0883 | n12c(nnc1c(cc3)ccn3)sc(c4ccc(c(Br)c4)F)n2 |
| Targeted | | | |
| Diversity Library | | | |
| ChemDiv | ChemDiv | D727-0915 | n12c(nnc1c3cc(n[nH]3)C)sc(c4cc(on4)C)n2 |
| Targeted | | | |
| Diversity Library | | | |
| ChemDiv | ChemDiv | E198-0044 | S(═O)(═O)(c(ccc(c1C2(C)C)N(C2═O)C)c1)N(CC3)CCN3c4ccc(cc4) |
| Targeted | | | Cl |
| Diversity Library | | | |
| ChemDiv | ChemDiv | E218-0327 | N1(c2ccc(cc2C)C)C(═O)c3n(CC1(C)C(═O)NC4CCC(CC4)C)c5c(occ5) |
| Targeted | | | c3 |
| Diversity Library | | | |
| ChemDiv | ChemDiv | E218-0329 | N1(c2c(OC)ccc(OC)c2)C(═O)c3n(CC1(C)C(═O)NC4CCC(CC4)C) |
| Targeted | | | c5c(occ5)c3 |
| Diversity Library | | | |
| ChemDiv | ChemDiv | E234-0004 | N(Cc(cccn1)c1)(C2═O)C(C)(Cn(c3c(cccc3)c4)c24)C(═O)NC5CCCCC5 |
| Targeted | | | |
| Diversity Library | | | |
| ChemDiv | ChemDiv | E218-0181 | n1(CC2(C)C(═O)NC3CCCCC3)c(cc(oc(C)c4)c14)C(N2Cc(ccc(c56) |
| Targeted | | | OCO5)c6)═O |
| Diversity Library | | | |
| ChemDiv | ChemDiv | E218-0296 | C(C)(C1)(C(═O)NC2CCC(CC2)C)N(c3ccc(c(OC)c3)OC)C(c4n1c5c |
| Targeted | | | (c4)occ5)═O |
| Diversity Library | | | |
| ChemDiv | ChemDiv | E234-0006 | C1(C)(Cn(c2c(c3)cccc2)c3C(═O)N1c4cccc(OC)c4)C(═O)NC5CCCCC5 |
| Targeted | | | |
| Diversity Library | | | |
| ChemDiv | ChemDiv | E218-0324 | N1(c2cccc(Cl)c2C)C(═O)c3n(CC1(C)C(═O)NC4CCC(CC4)C)c5c |
| Targeted | | | (occ5)c3 |
| Diversity Library | | | |
| ChemDiv | ChemDiv | E534-0255 | c1(CN(CC2)CCN2Cc(ccc(c34)OCO3)c4)csc(C)c1CC |
| Targeted | | | |
| Diversity Library | | | |
| ChemDiv | ChemDiv | E722-2588 | c12c(CSC(C(NCCc(cc3)ccc3S(N)(═O)═O)═O)═C1)c4c(CCCC4)s2 |
| Targeted | | | |
| Diversity Library | | | |
| ChemDiv | ChemDiv | E722-2652 | c12c(CSC(C(NCCCN(CC3)CCC3N4CCCCC4)═O)═C1)c5c(CCCC5)s2 |
| Targeted | | | |
| Diversity Library | | | |
| ChemDiv | ChemDiv | E922-0258 | c(cnn1c2ccc(cc2)C)(C(═O)Nc(cc3)ccc3Br)c1C(CC4)CCN4 |
| Targeted | | | |
| Diversity Library | | | |
| ChemDiv | ChemDiv | F083-0017 | N1(c2c(cc(Cl)cc2)C(N3)═O)C3═C(SC1═S)C(═O)Nc4ccc(cc4C)C |
| Targeted | | | |
| Diversity Library | | | |
| ChemDiv | ChemDiv | F086-0619 | c1(cc(ccc1N(CC2)CCN2c3ccccn3)C(O)═O)NC(═O)c4ccc(cc4)Br |
| Targeted | | | |
| Diversity Library | | | |
| ChemDiv | ChemDiv | F086-0033 | C(═O)(Nc1cc(ccc1N(CC2)CCN2CC(NCCCOC)═O)C(O)═O)c3ccccc3Cl |
| Targeted | | | |
| Diversity Library | | | |
| ChemDiv | ChemDiv | F083-0067 | N1(c2c(ccc(Cl)c2)C(N3)═O)C3═C(SC1═S)C(N([H])[H])═O |
| Targeted | | | |
| Diversity Library | | | |
| ChemDiv | ChemDiv | F083-0005 | N1(c2c(cc(Cl)cc2)C(N3)═O)C3═C(SC1═S)C(N([H])[H])═O |
| Targeted | | | |
| Diversity Library | | | |
| ChemDiv | ChemDiv | F086-0004 | C(═O)(Nc1cc(ccc1N(CC2)CCN2CCC)C(O)═O)c3ccccc3Cl |
| Targeted | | | |
| Diversity Library | | | |
| ChemDiv | ChemDiv | F086-0028 | C(═O)(Nc1cc(ccc1N(CC2)CCN2CC(═O)NC(C)C)C(O)═O)c3ccccc3Cl |
| Targeted | | | |
| Diversity Library | | | |
| ChemDiv | ChemDiv | F083-0404 | C1(═C(C(═O)N2CCCC2)SC3═S)N3c4c(cc(c5c4)OCO5)C(═O)N1 |
| Targeted | | | |
| Diversity Library | | | |
| ChemDiv | ChemDiv | F086-0029 | C(═O)(Nc1cc(ccc1N(CC2)CCN2CC(NCCCC)═O)C(O)═O)c3ccccc3Cl |
| Targeted | | | |
| Diversity Library | | | |
| ChemDiv | ChemDiv | F128-0076 | N1(c2ccc(cc2)Cl)C(═S)SC(C(═O)NCC3CCCO3)═C1N |
| Targeted | | | |
| Diversity Library | | | |
| ChemDiv | ChemDiv | F128-0041 | C(C(═O)N1CCCC1)(SC(N2Cc3ccccc3)═S)═C2N |
| Targeted | | | |
| Diversity Library | | | |
| ChemDiv | ChemDiv | F233-0200 | N1(c2ccccc2C)C(═O)C═C(C(C(═O)NC(c3ccccc3)c4ccccc4)═N1)OC |
| Targeted | | | |
| Diversity Library | | | |
| ChemDiv | ChemDiv | F293-0010 | S(═O)(═O)(Nc(cnc(c1C(O)═O)N(CC)Cc2ccccc2)c1)c3cc(C)ccc3C |
| Targeted | | | |
| Diversity Library | | | |
| ChemDiv | ChemDiv | F293-0458 | S(═O)(═O)(Nc(cnc(c1C(O)═O)N2CCCC2)c1)c3c(C)cc(c(C)c3)C |
| Targeted | | | |
| Diversity Library | | | |
| ChemDiv | ChemDiv | F293-0589 | S(═O)(═O)(Nc(cnc(c1C(O)═O)N(CC(C)C)CC(C)C)c1)c2c(C)cc(c(C) |
| Targeted | | | c2)C |
| Diversity Library | | | |
| ChemDiv | ChemDiv | F293-0814 | S(═O)(═O)(Nc(cnc(c1C(O)═O)N(C)C)c1)c2c(F)ccc(F)c2 |
| Targeted | | | |
| Diversity Library | | | |
| ChemDiv | ChemDiv | F293-0183 | c1(cc(cnc1N(CC2)CCC2(C(N)═O)N3CCCCC3)NS(C)(═O)═O)C |
| Targeted | | | (O)═O |
| Diversity Library | | | |
| ChemDiv | ChemDiv | F293-0515 | S(═O)(═O)(Nc(cnc(c1C(O)═O)N(CC)CCN(C)C)c1)c2c(C)cc(c(C)c2)C |
| Targeted | | | |
| Diversity Library | | | |
| ChemDiv | ChemDiv | F293-0898 | c1(cc(cnc1N2CCC(CC2)CCN(CC3)CCO3)NS(C)(═O)═O)C(O)═O |
| Targeted | | | |
| Diversity Library | | | |
| ChemDiv | ChemDiv | F293-0004 | S(c1ccccc1)(═O)(═O)Nc(cnc(c2C(O)═O)N(CC)Cc3ccccc3)c2 |
| Targeted | | | |
| Diversity Library | | | |
| ChemDiv | ChemDiv | F293-0908 | n1c(C)c(c(C)n1C)CCCN(C)c2ncc(cc2C(O)═O)NS(CC)(═O)═O |
| Targeted | | | |
| Diversity Library | | | |
| ChemDiv | ChemDiv | F293-0740 | S(═O)(═O)(Nc(cnc(c1C(O)═O)N(CC)CC)c1)c2c(C)cc(c(C)c2)C |
| Targeted | | | |
| Diversity Library | | | |
| ChemDiv | ChemDiv | F293-0962 | S(═O)(═O)(N(C)C)Nc(cnc(c1C(O)═O)N2CCC(CC2)CCN3CCCCC3) |
| Targeted | | | c1 |
| Diversity Library | | | |
| ChemDiv | ChemDiv | F294-0900 | S(═O)(═O)(N(C)C)Nc(ccc(c1C(O)═O)N2CCC(CC2)CCN(CC3)CCO3) |
| Targeted | | | c1 |
| Diversity Library | | | |
| ChemDiv | ChemDiv | F293-0006 | c1(cc(cnc1N(CC)Cc2ccccc2)NS(CC)(═O)═O)C(O)═O |
| Targeted | | | |
| Diversity Library | | | |
| ChemDiv | ChemDiv | F293-0426 | S(═O)(═O)(Nc(cnc(c1C(O)═O)N2CCCC2)c1)c3c(C)cc(cc3C)C |
| Targeted | | | |
| Diversity Library | | | |
| ChemDiv | ChemDiv | F294-0983 | S(═O)(═O)(N(C)C)Nc(ccc(c1C(O)═O)N2CCCC(CN3CCCC3)C2)c1 |
| Targeted | | | |
| Diversity Library | | | |
| ChemDiv | ChemDiv | F305-0030 | C(CCCN1c2ccc(nn2)c3ccccc3)(C1)C(═O)N(CCCC)CC |
| Targeted | | | |
| Diversity Library | | | |
| ChemDiv | ChemDiv | F294-0002 | S(═O)(═O)(Nc(ccc(c1C(O)═O)N(CC)Cc2ccccc2)c1)c3c(C)cc |
| Targeted | | | (cc3C)C |
| Diversity Library | | | |
| ChemDiv | ChemDiv | F294-1002 | S(═O)(═O)(Nc(ccc(c1C(O)═O)N2CCCC(CN3CCCC3)C2)c1)N(CC4) |
| Targeted | | | CCO4 |
| Diversity Library | | | |
| ChemDiv | ChemDiv | F293-0441 | S(═O)(═O)(c1ccc(c2c1)CCCC2)Nc(cnc(c3C(O)═O)N4CCCC4)c3 |
| Targeted | | | |
| Diversity Library | | | |
| ChemDiv | ChemDiv | F293-0563 | c1(cc(cnc1N(CC(C)C)CC(C)C)NS(CC)(═O)═O)C(O)═O |
| Targeted | | | |
| Diversity Library | | | |
| ChemDiv | ChemDiv | F294-0003 | S(═O)(═O)(c1ccc(cc1)OC)Nc(ccc(c2C(O)═O)N(CC)Cc3ccccc3)c2 |
| Targeted | | | |
| Diversity Library | | | |
| ChemDiv | ChemDiv | F401-0259 | c12n(c(nn1)SCc3ccccc3)c(c4C(═O)N2CCc5ccccc5)ccs4 |
| Targeted | | | |
| Diversity Library | | | |
| ChemDiv | ChemDiv | F388-0145 | C1(═O)c2c(cccc2)N═CN1CCC(═O)NC3CCCc(cccc4)c34 |
| Targeted | | | |
| Diversity Library | | | |
| ChemDiv | ChemDiv | F388-0151 | C1(═O)c2c(cccc2)N═CN1CCC(═O)Nc(ccc(c34)OCCO3)c4 |
| Targeted | | | |
| Diversity Library | | | |
| ChemDiv | ChemDiv | F449-1274 | n12c(sc(N(C)CC(NCCN(CC)CC)═O)n1)nc(c3ccc(cc3)OC)c2NC4CCCC4 |
| Targeted | | | |
| Diversity Library | | | |
| ChemDiv | ChemDiv | F458-0083 | c12n(c(nn1)SCc(cc3)ccc3C═C)c(cccc4)c4C(═O)N2Cc5ccccc5 |
| Targeted | | | |
| Diversity Library | | | |
| ChemDiv | ChemDiv | F518-0008 | n1(ncn2)c2nc(C)cc1Sc3ccccc3NC(═O)c4cccc(F)c4 |
| Targeted | | | |
| Diversity Library | | | |
| ChemDiv | ChemDiv | F545-0052 | n1c(C)onc1c(cccc2C(═O)Nc3ccccc3CC)c2 |
| Targeted | | | |
| Diversity Library | | | |
| ChemDiv | ChemDiv | F571-0021 | N12C(═CC(═O)N1)N═C(c3ccccc3)N═C2SCC(═O)Oc4ccc(cc4C)C |
| Targeted | | | |
| Diversity Library | | | |
| ChemDiv | ChemDiv | F585-0060 | c12c(cccc1c3ccc(cc3)F)c(C(NCCN4CCCCC4)═O)cc(c5ccc(cc5) |
| Targeted | | | OC)n2 |
| Diversity Library | | | |
| ChemDiv | ChemDiv | F585-0086 | c12c(cccc1c3ccc(cc3)F)c(C(NCCN(CC4)CCO4)═O)cc(c5ccc(cc5) |
| Targeted | | | OC)n2 |
| Diversity Library | | | |
| ChemDiv | ChemDiv | F646-0578 | n1(c2c(C)ccc(c2)C(NCCc(c3)c4c([nH]3)ccc(Cl)c4)═O)c5c(nn1) |
| Targeted | | | cccn5 |
| Diversity Library | | | |
| ChemDiv | ChemDiv | F646-0636 | n1(c2c(C)ccc(c2)C(═O)NC(C)c(ccc(c34)OCCO3)c4)c5c(nn1) |
| Targeted | | | cccn5 |
| Diversity Library | | | |
| ChemDiv | ChemDiv | F640-0126 | S(NCCOC)(═O)(═O)c(c[nH]c1c2oc(nn2)C)c1 |
| Targeted | | | |
| Diversity Library | | | |
| ChemDiv | ChemDiv | F686-0287 | S(CC)(═O)(═O)Nc1ccc(cc1C(O)═O)N2CCCC2 |
| Targeted | | | |
| Diversity Library | | | |
| ChemDiv | ChemDiv | F685-0939 | c1(C(O)═O)cc(ccc1NC(═O)C2CC2)N3CCCC3 |
| Targeted | | | |
| Diversity Library | | | |
| ChemDiv | ChemDiv | F685-0437 | c1(C(O)═O)cc(ccc1NC(C(C)C)═O)N2CCCC2 |
| Targeted | | | |
| Diversity Library | | | |
| ChemDiv | ChemDiv | F685-1588 | c1(C(O)═O)cc(ccc1NC(C2CCCC2)═O)N3CCCC3 |
| Targeted | | | |
| Diversity Library | | | |
| ChemDiv | ChemDiv | F727-1225 | S(═O)(═O)(N(CC1)CCN1c2ncnc(c2)c3cc(F)ccc3OC)c(cnn4C(F)F) |
| Targeted | | | c4C |
| Diversity Library | | | |
| ChemDiv | ChemDiv | F727-1233 | S(═O)(═O)(c1ccc(c(Cl)c1)F)N(CC2)CCN2c3ncnc(c3)c4cc(F) |
| Targeted | | | ccc4OC |
| Diversity Library | | | |
| ChemDiv | ChemDiv | F793-0010 | c(C(═O)Nc1cccc(F)c1)(nnc2C3CCCN3C(═O)Nc(ccc(c4Cl)C)c4)s2 |
| Targeted | | | |
| Diversity Library | | | |
| ChemDiv | ChemDiv | F793-0016 | c(C(═O)Nc1cccc(F)c1)(nnc2C3CCCN3C(═O)Nc4ccc(c(OC)c4)OC) |
| Targeted | | | s2 |
| Diversity Library | | | |
| ChemDiv | ChemDiv | F835-0569 | n12c(C(NN═C1SC)═O)cc(c3cccs3)n2 |
| Targeted | | | |
| Diversity Library | | | |
| ChemDiv | ChemDiv | F854-0008 | c1(C(═O)Nc(cccc2C(═O)NCc(cccn3)c3)c2)sc(nn1)CC |
| Targeted | | | |
| Diversity Library | | | |
| ChemDiv | ChemDiv | F869-1268 | n1c(c2ccccc2)nccc1N(CC3)CCC3C(═O)NC(C)CC |
| Targeted | | | |
| Diversity Library | | | |
| ChemDiv | ChemDiv | F854-0333 | c1(C(═O)Nc(cccc2C(═O)N(CC3)CCN3c4ccccn4)c2)sc(nn1)C |
| Targeted | | | |
| Diversity Library | | | |
| ChemDiv | ChemDiv | G199-0400 | N12C(═NC(CSC3═NC(c4ccc(cc4)C)═NC(═CC(═O)N5)N35)═CC1═O) |
| Targeted | | | SC(C6CC6)═N2 |
| Diversity Library | | | |
| ChemDiv | ChemDiv | G226-0500 | c1(C(OC)═O)sc(c2c1S(N)(═O)═O)cccc2 |
| Targeted | | | |
| Diversity Library | | | |
| ChemDiv | ChemDiv | G786-1562 | c1(sc(c(ccc2S(NC)(═O)═O)n1)c2)NC(═O)c(cc3)ccc3N4C(═O) |
| Targeted | | | CCC4═O |
| Diversity Library | | | |
| ChemDiv | ChemDiv | G786-0335 | c(C(c1ccccc1)═O)(s2)c(c3ccccc3)nc2NC(═O)c(ccc(c45)OCCO4) |
| Targeted | | | c5 |
| Diversity Library | | | |
| ChemDiv | ChemDiv | G784-0958 | c12c(c(nn1c3cccc(F)c3)C)cc(C(═O)Oc(ccc(c4ccn5)c5)c4)s2 |
| Targeted | | | |
| Diversity Library | | | |
| ChemDiv | ChemDiv | G786-1547 | c1(sc(c(ccc2S(N)(═O)═O)n1)c2)NC(═O)c3ccccc3C |
| Targeted | | | |
| Diversity Library | | | |
| ChemDiv | ChemDiv | G843-1071 | C(C═CC(N1Cc2ccccc2F)═O)(C(═O)Nc(cc3)ccc3C(OCC)═O)═C1 |
| Targeted | | | |
| Diversity Library | | | |
| ChemDiv | ChemDiv | G856-6165 | N1(c2ccc(cc2)C)C(═S)SC(C(═O)NCC3CCCO3)═C1N |
| Targeted | | | |
| Diversity Library | | | |
| ChemDiv | ChemDiv | G857-2274 | c12c(ncnc1N3CCC(CC3)O)n(nn2)CC |
| Targeted | | | |
| Diversity Library | | | |
| ChemDiv | ChemDiv | G889-0745 | c1(cc(ccc1N(C)CC(OCC)═O)NC(C)═O)C(O)═O |
| Targeted | | | |
| Diversity Library | | | |
| ChemDiv | ChemDiv | G946-0149 | C1(═O)c2c(cccc2)Sc(cc3c4noc(CN5c6c(cccc6)OCC5═O)n4)c(cc3) |
| Targeted | | | N1C |
| Diversity Library | | | |
| ChemDiv | ChemDiv | J094-0187 | n1(nc(c(C(CC(═O)N2)c(cc3)ccc3C(O)═O)c12)C)c4[nH]c(c5n4) |
| Targeted | | | cccc5 |
| Diversity Library | | | |
| ChemDiv | ChemDiv | K261-1443 | S1(═O)(═O)c2c(cccc2)NC(SCc(cc3)ccc3C═C)═N1 |
| Targeted | | | |
| Diversity Library | | | |
| ChemDiv | ChemDiv | G373-2168 | n(ccn1)(c1)C(═O)c(ccc2c3OCC(═O)N2)c3 |
| Targeted | | | |
| Diversity Library | | | |
| ChemDiv | ChemDiv | G373-2873 | N1C(COc2c1ccc(c2)C)CC(O)═O |
| Targeted | | | |
| Diversity Library | | | |
| ChemDiv | ChemDiv | G517-0062 | c1(c(C)c(s2)C)c2nc(C)nc1Oc3cccc(Cl)c3 |
| Targeted | | | |
| Diversity Library | | | |
| ChemDiv | ChemDiv | G517-0063 | c1(c(C)c(s2)C)c2nc(C)nc1Oc3cccc(F)c3 |
| Targeted | | | |
| Diversity Library | | | |
| ChemDiv | ChemDiv | G517-0064 | c1(c(C)c(s2)C)c2nc(C)nc1Oc(cc3)ccc3F |
| Targeted | | | |
| Diversity Library | | | |
| ChemDiv | ChemDiv | G517-0076 | c1(c(C)c(s2)C)c2nc(C)nc1Oc(cc3)ccc3OCC |
| Targeted | | | |
| Diversity Library | | | |
| ChemDiv | ChemDiv | G517-0078 | c1(c(C)c(s2)C)c2nc(C)nc1Oc(cc3)ccc3OCCC |
| Targeted | | | |
| Diversity Library | | | |
| ChemDiv | ChemDiv | G620-0592 | S(═O)(═O)(Nc(cc1)ccc1c(ccc(n2)N(CC3)CCO3)n2)c4c(OC)ccc(OC) |
| Targeted | | | c4 |
| Diversity Library | | | |
| ChemDiv | ChemDiv | G620-0632 | S(═O)(═O)(Nc(cc1)ccc1c(ccc(n2)N(CC3)CCO3)n2)c4ccc(cc4OC) |
| Targeted | | | OC |
| Diversity Library | | | |
| ChemDiv | ChemDiv | G620-0536 | S(═O)(═O)(Nc(cc1)ccc1c(ccc(n2)N3CCCC3)n2)c4c(OC)ccc(OC) |
| Targeted | | | c4 |
| Diversity Library | | | |
| ChemDiv | ChemDiv | G650-0193 | C1(C(═O)Nc2nccc(C)n2)═C(O)c3c(N(CC)C1═O)cccc3 |
| Targeted | | | |
| Diversity Library | | | |
| ChemDiv | ChemDiv | G713-0011 | c1(C(═O)Nc(cc2)ccc2C)sc(nn1)CCC(═O)Nc3ccc(cc3OC)OC |
| Targeted | | | |
| Diversity Library | | | |
| ChemDiv | ChemDiv | G702-4364 | n1(nc(n2)c3ccco3)c2nc(CCC4)c4c1NCc5cccs5 |
| Targeted | | | |
| Diversity Library | | | |
| ChemDiv | ChemDiv | G721-0011 | N(C)(C(═O)c1c(N2C)ncc(C(C)C)c1SC3CCCC3)C2═O |
| Targeted | | | |
| Diversity Library | | | |
| ChemDiv | ChemDiv | L150-0826 | c1(cn(c(ccc2S(═O)(═O)N(C)C)c1c2)CC)C(═O)n(ccn3)c3 |
| Targeted | | | |
| Diversity Library | | | |
| ChemDiv | ChemDiv | L150-0160 | n1(ncn2)c2nc(CCC)cc1Sc3ccccc3N |
| Targeted | | | |
| Diversity Library | | | |
| ChemDiv | ChemDiv | L150-0829 | c1(cn(c(ccc2S(═O)(═O)N(C)C)c1c2)C)C(═O)n(ccn3)c3 |
| Targeted | | | |
| Diversity Library | | | |
| ChemDiv | ChemDiv | L378-0350 | c1(N(CC2)CCN2C(═O)Nc3cccc(C)c3)nnnn1c4cccc(CC)c4 |
| Targeted | | | |
| Diversity Library | | | |
| ChemDiv | ChemDiv | L378-0355 | c1(N(CC2)CCN2C(═O)Nc3ccc(cc3C)C)nnnn1c4cccc(CC)c4 |
| Targeted | | | |
| Diversity Library | | | |
| ChemDiv | ChemDiv | L378-0372 | c1(N(CC2)CCN2C(═O)Nc(ccc(c3C)Br)c3)nnnn1c4cccc(CC)c4 |
| Targeted | | | |
| Diversity Library | | | |
| ChemDiv | ChemDiv | L663-1002 | C(═O)(c1cccc(c1)Oc2ncnc(c3ccccc3)c2)NC4CCCC4 |
| Targeted | | | |
| Diversity Library | | | |
| ChemDiv | ChemDiv | L663-1076 | c1c(c2ccccc2)ncnc1Oc(cccc3C(═O)NCc(ccc(c45)OCO4)c5)c3 |
| Targeted | | | |
| Diversity Library | | | |
| ChemDiv | ChemDiv | L662-0973 | n1c(Oc(cc2)ccc2C(═O)NCc3cc(OC)ccc3OC)ccnc1c4cccc(F)c4 |
| Targeted | | | |
| Diversity Library | | | |
| ChemDiv | ChemDiv | L662-0977 | n1c(Oc(cc2)ccc2C(═O)NCc(cc3)ccc3F)ccnc1c4cccc(F)c4 |
| Targeted | | | |
| Diversity Library | | | |
| ChemDiv | ChemDiv | L663-1062 | C(═O)(c1cccc(c1)Oc2ncnc(c3ccccc3)c2)NC(C)c4ccc(cc4)C |
| Targeted | | | |
| Diversity Library | | | |
| ChemDiv | ChemDiv | L662-0597 | n1c(Oc(cc2)ccc2C(═O)NCC(C)C)ccnc1c3ccc(cc3)F |
| Targeted | | | |
| Diversity Library | | | |
| ChemDiv | ChemDiv | L662-0654 | n1c(Oc(cc2)ccc2C(═O)Nc(ccc(c3Cl)F)c3)ccnc1c4ccc(cc4)F |
| Targeted | | | |
| Diversity Library | | | |
| ChemDiv | ChemDiv | L662-0987 | n1c(Oc(cc2)ccc2C(═O)NCc3ccc(cc3OC)OC)ccnc1c4cccc(F)c4 |
| Targeted | | | |
| Diversity Library | | | |
| ChemDiv | ChemDiv | L675-0238 | C1(NCc(cc2)ccc2OC)═NCCNC13CCCCC3C |
| Targeted | | | |
| Diversity Library | | | |
| ChemDiv | ChemDiv | L663-0996 | C(═O)(c1cccc(c1)Oc2ncnc(c3ccccc3)c2)NC(C)CC |
| Targeted | | | |
| Diversity Library | | | |
| ChemDiv | ChemDiv | L662-0604 | n1c(Oc(cc2)ccc2C(═O)NCC3CCCO3)ccnc1c4ccc(cc4)F |
| Targeted | | | |
| Diversity Library | | | |
| ChemDiv | ChemDiv | L663-0999 | C(═O)(c1cccc(c1)Oc2ncnc(c3ccccc3)c2)NC4CCCCC4 |
| Targeted | | | |
| Diversity Library | | | |
| ChemDiv | ChemDiv | L705-0872 | S(═O)(═O)(CC(C)C(═O)N1CCc(c2C1)cccc2)c(ccc(c34)SCCC(═O) |
| Targeted | | | N3)c4 |
| Diversity Library | | | |
| ChemDiv | ChemDiv | L921-0265 | c1(c(C)sc(c2scc(C)n2)c1)S(═O)(═O)NCc(cc3)ccc3N(C)C |
| Targeted | | | |
| Diversity Library | | | |
| ChemDiv | ChemDiv | M056-0617 | c1(ccn2CC(═O)NCc(cc3)ccc3N(CC4)CCN4C)c2ccnc1SCc5ccccc5 |
| Targeted | | | |
| Diversity Library | | | |
| ChemDiv | ChemDiv | M130-0012 | c1(ncnc(c2ccc(cc2)OC)c1)N(CC3)CCN3C(═O)c(cc4)ccc4C(C)(C) |
| Targeted | | | C |
| Diversity Library | | | |
| ChemDiv | ChemDiv | M467-0616 | S(═O)(═O)(c1ccc(c2c1)CCC2)Nc3onc(c4ccoc4)c3 |
| Targeted | | | |
| Diversity Library | | | |
| ChemDiv | ChemDiv | Z354-0947 | c1(N2CCC(CC2)C(NCCC)═O)c3c(csc3C)nc(C(C4)CC4)n1 |
| Targeted | | | |
| Diversity Library | | | |
| Enamine 2 | Enamine | T5324509 | CCC(CC)NC(═O)c1ccc2ncsc2c1 |
| Enamine 2 | Enamine | T5253378 | OC(═O)c1cc(N/N═C2\SC(═N)N═C/2)c(Cl)cc1 |
| Enamine 2 | Enamine | T5253322 | CC(═O)Oc1c(cccc1OC(═O)C)OC(═O)C |
| Enamine 2 | Enamine | T5221033 | O═c1cc(CSc2nnc(Cc3ccccc3F)n2N)c2cc(O)c(O)cc2o1 |
| Enamine 2 | Enamine | T0510-5997 | Cc1sc2ncnc(N)c2c1C |
| Enamine 2 | Enamine | T5275200 | O═C(COC(═O)c1cc(nc2ccccc12)c1ccco1)N1CCCC1 |
| Enamine 2 | Enamine | T0509-4263 | OC(═O)c1ccccc1NS(═O)(═O)c1cccc(c1)n1sc2ccccc2c1═O |
| Enamine 2 | Enamine | T0510-1246 | CCCCC(═C1C(═O)CCCC1═O)N1CCCC1 |
| Enamine 2 | Enamine | T0515-6189 | O═C(CSc1nnc[nH]1)c1cccc(c1)C(F)(F)F |
| Enamine 2 | Enamine | T0515-6232 | CC1═CC(═O)C(═C\C/1═N/C(═O)c1ccco1)C |
| Enamine 2 | Enamine | T0507-8510 | O═C1c2ccccc2C(═O)N1SC1CCCCC1 |
| Enamine 2 | Enamine | T0520-4712 | CCOC(═O)c1c(CSc2cccc[n+]2[O—])[nH]c(C(═O)OCC)c1C |
| Enamine 2 | Enamine | T0507-9032 | CC(CCCNc1nnc(S)s1)CC |
| Enamine 2 | Enamine | T0509-1852 | S═C(N/N═C/c1ccc(O)c(O)c1O)Nc1ccc(Cl)cc1Cl |
| Enamine 2 | Enamine | T0500-6341 | Cc1cc(C)[nH]c(═S)c1C#N |
| Enamine 2 | Enamine | T0502-6915 | Cc1ccc(cc1)NP(═O)(C)c1nc2CCCCc2s1 |
| Enamine 2 | Enamine | T5463073 | N#CCCn1nc(cc1Nc1ccccc1N)c1ccccc1 |
| Enamine 2 | Enamine | T5233568 | CC(═O)OCc1ccc(/C═C\C(═O)O)o1 |
| Enamine 2 | Enamine | T5483278 | Nc1nc(N)c(N)c(═O)n1C |
| Enamine 2 | Enamine | T0509-3972 | Clc1cc(CN2CCCCCC2)c(O)c2ncccc12 |
| Enamine 2 | Enamine | T5501904 | S═c1[nH]nc(s1)NC(C)(C)C |
| Enamine 2 | Enamine | T5412600 | CCOC(═O)c1sc(N)c(C#N)c1COC(═O)CC1Sc2ccccc2NC1═O |
| Enamine 2 | Enamine | T5471321 | O═C(OCC(═O)Nc1cccc2nsnc12)CCc1c[nH]c2ccccc12 |
| Enamine 2 | Enamine | T0520-0702 | Oc1ccc(cc1)N1CCN(CC1)CC(═O)NC1CCCc2ccccc12 |
| Enamine 2 | Enamine | T5238843 | CC1OC(C)CN(C1)Cn1nc(c2cccs2)n(c2ccccc2)c1═S |
| Enamine 2 | Enamine | T0519-4497 | C═CCN(CC═C)Cn1nc([nH]c1═S)c1cccs1 |
| Enamine 2 | Enamine | T0519-5245 | S═c1n(CN2CCCC2)nc2sc3ccccc3n12 |
| Enamine 2 | Enamine | T0519-9012 | Clc1ccc(C)c(c1)NC(═O)C1═NCCN1 |
| Enamine 2 | Enamine | T0519-8609 | OC(═O)\C═C/c1cc(C)n(c2ccccc2)c1C |
| Enamine 2 | Enamine | T0519-9021 | O═C(Nc1ccc(cc1)N1CCOCC1)C1═NCCN1 |
| Enamine 2 | Enamine | T5238087 | CC1OC(C)CN(C1)Cn1nc(c2ccc(Br)o2)n(Cc2ccccc2)c1═S |
| Enamine 2 | Enamine | T0519-9962 | CC(═NNC(═O)Cc1nc2sccn2c1)C |
| Enamine 2 | Enamine | T0519-5214 | Oc1ccc(cc1)N1CCN(CC1)C(c1ccccc1)C(═O)c1c[nH]c2ccccc12 |
| Enamine 2 | Enamine | T5237824 | Oc1ccc2c(COC(═O)c3cccc(c3)N(C)C)cc(═O)oc2c1 |
| Enamine 2 | Enamine | T0519-5941 | CN(Cn1nc2sc3ccccc3n2c1═S)C1CCCCC1 |
| Enamine 2 | Enamine | T5342193 | Cc1nc(S)n(C2CCCCC2)c1C |
| Enamine 2 | Enamine | T0518-5977 | OC(═O)c1oc2ccccc2c1CSC1═NCCS1 |
| Enamine 2 | Enamine | T5342236 | CCn1c(S)ncc1c1ccccc1 |
| Enamine 2 | Enamine | T5449407 | Fc1ccc(cc1)OCc1n[nH]c(═S)n1N |
| Enamine 2 | Enamine | T5343027 | NNC(═O)CSC(═S)N1CCCC1 |
| Enamine 2 | Enamine | T5343029 | NC(═S)C(═O)Nc1ccccc1C |
| Enamine 2 | Enamine | T5342754 | Cc1ccc(cc1)Cc1cnc(S)s1 |
| Enamine 2 | Enamine | T0513-7645 | Fc1ccc(cc1)C(═O)NN1C(═S)SCC1═O |
| Enamine 2 | Enamine | T0519-8157 | Ccloccc1c1nnc(S)n1C |
| Enamine 2 | Enamine | T5342842 | CC(C)Cn1c(S)ncc1c1ccccc1 |
| Enamine 2 | Enamine | T5342230 | S═c1[nH]nc(o1)C1CC1 |
| Enamine 2 | Enamine | T0504-6366 | CCOC1CSc2sc(═S)sc2S1 |
| Enamine 2 | Enamine | T5466423 | S═C(NCC1CCCO1)SCc1cn2cccnc2n1 |
| Enamine 2 | Enamine | T0504-8752 | CC1═NN(CCc2ncc(s2)c2ccccc2)C(═O)\C/1═C1\CCCCCN\1 |
| Enamine 2 | Enamine | T0520-3537 | Cc1noc(C)c1CSc1nnc(c2ccccc2Br)n1Cc1ccccc1 |
| Enamine 2 | Enamine | T0400-1924 | COC(═O)NNc1ccccc1 |
| Enamine 2 | Enamine | T0520-4198 | CCCc1nc(SCC(═O)\C(═C(\C)/N)/C#N)nc(O)c1 |
| Enamine 2 | Enamine | T5360007 | CN(Cc1ccccc1)Cn1nc([nH]c1═S)c1cccs1 |
| Enamine 2 | Enamine | T0504-6236 | O═C(NCc1cn2ccsc2n1)Nc1ccc(cc1)Cc1ccc(cc1)NC(═O) |
| | | | NCc1cn2ccsc2n1 |
| Enamine 2 | Enamine | T0504-5648 | COC(═O)NP(═O)(OC)NNc1ccccc1 |
| Enamine 2 | Enamine | T5466422 | S═C(SCc1nc2ncccn2c1)NCc1ccco1 |
| Enamine 2 | Enamine | T0504-6551 | CCNC(═S)Nc1ccc2[nH]c(═O)[nH]c2c1 |
| Enamine 2 | Enamine | T5399920 | Cc1ccc(cc1)NC(═O)CN(C)C(═O)C1COc2ccccc2O1 |
| Enamine 2 | Enamine | T5350732 | O═C(Nc1nnc[nH]1)c1cccnc1 |
| Enamine 2 | Enamine | T5380152 | CCn1c(CCC(═O)O)nc2cc(ccc12)S(═O)(═O)N |
| Enamine 2 | Enamine | T5383254 | COCC/N═C/c1cc(Br)cc(Br)c1O |
| Enamine 2 | Enamine | T5356906 | CC1CCC2(CC1)NC(═O)N(NC(═S)NCc1ccc3OCOc3c1)C2═O |
| Enamine 2 | Enamine | T5385370 | S═c1[nH]ncc(n1)c1cccs1 |
| Enamine 2 | Enamine | T5465785 | Cc1ccc(cc1)c1n[nH]c(═S)[nH]c1═O |
| Enamine 2 | Enamine | T5446998 | N#Cc1sc2[nH]c(═O)c(C#N)c(SC)c2c1N |
| Enamine 2 | Enamine | T5448031 | N#Cc1cc(C#N)c(N)nc1SCc1csc(C)n1 |
| Enamine 2 | Enamine | T5444232 | O═C(NCC1COc2ccccc2O1)c1cc(nc2ccccc12)c1ccco1 |
| Enamine 2 | Enamine | T0519-6400 | CCOC(═O)C1═C(C)NN═C(S1)Nc1cccc(C)c1C |
| Enamine 2 | Enamine | T5440124 | O═C(CSc1nc2cc(ccc2n1C)S(═O)(═O)N)NC(═O)Nc1ccccc1F |
| Enamine 2 | Enamine | T0519-6842 | CCC(CC)NC(═O)CSc1nnc(c2ccc(C)cc2)n1N |
| Enamine 2 | Enamine | T0517-4540 | Oc1c(O)ccc(\C═N/n2c(C)cs\c\2═N\C2CCCCC2)c1O |
| Enamine 2 | Enamine | T0519-6231 | CNC(═S)N/N═C1\C(═Nc2ccc(cc/12)C(C)C)O |
| Enamine 2 | Enamine | T0519-6369 | NNC(═O)C(C)Oc1ccc2c(c1)oc(═O)cc2C(F)(F)F |
| Enamine 2 | Enamine | T5239935 | COc1cc(\C═N/n2cc(nc2S)c2ccccc2)cc(OC)c1OC(═O)C |
| Enamine 2 | Enamine | T5238978 | N#Cc1c(nc(N)c2c(N)nc(SCC(═O)O)cc12)N(C)c1ccccc1 |
| Enamine 2 | Enamine | T0519-5365 | O═c1[nH]c2ccccc2n2c(S)nnc12 |
| Enamine 2 | Enamine | T0519-7856 | Nn1c(S)nnc1c1ccco1 |
| Enamine 2 | Enamine | T0503-6223 | COc1ccc(cc1)n1c(N(C(═O)C)C(═O)C)c(C#N)c2nc3ccccc3nc12 |
| Enamine 2 | Enamine | T5441846 | CC(═O)SCc1n[nH]c(═S)n1C(═O)C |
| Enamine 2 | Enamine | T0503-7385 | COc1ccc(cc1)\C(═N\O)/COc1ccccc1O |
| Enamine 2 | Enamine | T0503-7720 | N#CCCn1nc(C)c2c1N═C(OP2(═S)N1CCCCC1)c1ccccc1 |
| Enamine 2 | Enamine | T5441221 | NNc1nc(nc2n(ncc12)Cc1ccccc1)C(F)(F)F |
| Enamine 2 | Enamine | T5441809 | O═C1NC(═S)C(═C2CCCC2)S1 |
| Enamine 2 | Enamine | T0503-7336 | [O—]C1NN═C(C(C)C1)c1ccc2[n+]c(COc3ccccc3O)[nH]c2c1 |
| Enamine 2 | Enamine | T0503-8014 | N#CC(C)(C)NNC(═S)N |
| Enamine 2 | Enamine | T5441192 | CC(C)CNc1n[H]c(═S)s1 |
| Enamine 2 | Enamine | T5539656 | O═C(N1CCN(CC1)C(═O)c1sc2nc[nH]c(═O)c2c1C)C1COc2ccccc2O1 |
| Enamine 2 | Enamine | T5441862 | COc1ccc(cc1)C1C2C(Sc3[nH]c(═O)sc13)C(═O)N(CC(═O)O)C2═O |
| Enamine 2 | Enamine | T5441199 | Cn1c(CC(═O)OCC)n[nH]c1═S |
| Enamine 2 | Enamine | T5441203 | O═c1oc2ccccc2o1 |
| Enamine 2 | Enamine | T5441826 | OC(═O)/C(═C\C═C/c1ccccc1)\S |
| Enamine 2 | Enamine | T5345839 | O═C(CN1CCN(CC1)c1ccccc1O)N(C)c1ccccc1 |
| Enamine 2 | Enamine | T5441843 | O═C1NC(═O)C(═C2CCCCC2)S1 |
| Enamine 2 | Enamine | T0503-6911 | CCn1c(═O)c2cccc3cccc1c23 |
| Enamine 2 | Enamine | T5248882 | COCCn1c(C)cc(/C═N\n2c(S)nnc2Cc2cccc3ccccc23)c1C |
| Enamine 2 | Enamine | T0516-1616 | O═c1[nH][nH]c(═S)n1C1CC1 |
| Enamine 2 | Enamine | T0518-8713 | O═C(NN═C1CCCC1)c1ccccc1NS(═O)(═O)c1cccs1 |
| Enamine 2 | Enamine | T5227570 | OC(═O)c1ccccc1Sc1ncnc2sccc12 |
| Enamine 2 | Enamine | T0518-7708 | CC(O)CNc1nc2ccccc2n1CC(═O)N(Cc1ccccc1)C(C)C |
| Enamine 2 | Enamine | T5227668 | CCOC(═O)C1CCCN(C1)C(═O)c1cc(nc2ccccc12)c1ccco1 |
| Enamine 2 | Enamine | T5227576 | OC(═O)CSc1ncnc2sccc12 |
| Enamine 2 | Enamine | T5229649 | CCc1ccc(cc1)NC(═O)/C(═C\c1cc(C#N)n(C)c1C)\C#N |
| Enamine 2 | Enamine | T5245627 | Oc1nnc2ccccn12 |
| Enamine 2 | Enamine | T5212958 | CN1CCC(CC1)N(C)Cc1cc(c(O)c(c1)C(C)(C)C)C(C)(C)C |
| Enamine 2 | Enamine | T0503-9777 | N#Cc1cc(c(C)n1C)P(═S)(N1CCOCC1)N1CCOCC1 |
| Enamine 2 | Enamine | T5211966 | Fc1ccc(cc1)n1nc(C)c(/C═N\N═C2\C(═O)Nc3ccccc/23)c1Cl |
| Enamine 2 | Enamine | T5245573 | COc1cccc(c1)Oc1ccc(cc1Nc1nc2ccccc2o1)C(F)(F)F |
| Enamine 2 | Enamine | T0504-1446 | C1═CSC(═C2SC═CS2)S1 |
| Enamine 2 | Enamine | T5237273 | O═c1n(Cc2ccccc2)c2nnc(S)n2c2ccccc12 |
| Enamine 2 | Enamine | T5241045 | N#C/C(═C(/C)\N1CCCC1)/P(═N\N═N\c1ccccc1)(N1CCOCC1) |
| | | | N1CCOCC1 |
| Enamine 2 | Enamine | T5245636 | O═C(Nc1c(cnn1C(═S)N)C(═O)O)c1ccccc1Cl |
| Enamine 2 | Enamine | T5237227 | COc1ccc(cc1)NCc1c(C#N)c(C)nn1c1ccccc1 |
| Enamine 2 | Enamine | T5237274 | COc1ccccc1C(═S)N |
| Enamine 2 | Enamine | T0504-1173 | Cc1ccc(cc1)N1C(═O)c2ccccc2C1C(═O)c1ccc(O)cc1O |
| Enamine 2 | Enamine | T0519-0594 | O═C(CSc1ncnc2[nH]ncc12)CSc1ncnc2[nH]ncc12 |
| Enamine 2 | Enamine | T5212942 | Oc1ccc(cc1)N1CCN(CC1)C1CC(═O)N(c2ccc(Cl)c(c2)C(F)(F)F)C1═O |
| Enamine 2 | Enamine | T5237295 | Cc1ccc(cc1)S(═O)(═O)/C═C1\SCC(═O)N\1 |
| Enamine 2 | Enamine | T0504-1223 | CC(C)CC1NC(═S)N(Cc2ccccc2)C1═O |
| Enamine 2 | Enamine | T0504-2231 | CCn1c2ccccc2nc1P(═S)(N(CC)CC)N(CC)CC |
| Enamine 2 | Enamine | T5245560 | O═C(COC(═O)C1CC(═O)N(Cc2ccccc2)C1)Nc1ccc2OCOc2c1 |
| Enamine 2 | Enamine | T0513-9090 | OC(═O)CCCCN1C(═S)SCC1═O |
| Enamine 2 | Enamine | T0504-1405 | CCOC(═O)C1═C(C)C\C(═N/NC(═O)c2ccccc2O)\CC1 |
| Enamine 2 | Enamine | T0513-0218 | CCOC(═O)C1CCCN(C1)Cn1nnn(c2ccc(OC)cc2)c1═S |
| Enamine 2 | Enamine | T5211003 | OC(═O)CSc1nnc(NC2CCCCC2)s1 |
| Enamine 2 | Enamine | T0520-0461 | S═c1[nH]c2ccccc2c(═O)n1c1cccc(c1)S(═O)(═O)N1CCCC1 |
| Enamine 2 | Enamine | T5224714 | S═C═Nc1ccccn1 |
| Enamine 2 | Enamine | T0520-2027 | CNC(═S)N/N═C/c1cccc(Cl)c1 |
| Enamine 2 | Enamine | T5213804 | O═C1CSc2ccc(cc2N1)C(═O)N1CCN(CC1)Cc1ccc2OCOc2c1 |
| Enamine 2 | Enamine | T0520-0462 | Cc1sc2nc3CCCn3c(═O)c2c1c1ccccc1 |
| Enamine 2 | Enamine | T0513-0809 | CCNC(═S)Nc1ccc(cc1)N1CCCCC1 |
| Enamine 2 | Enamine | T5213542 | S═C1NC(═O)/C(═C\c2ccc(o2)c2ccc(cc2)S(═O)(═O)N2CCOCC2)\ |
| | | | S1 |
| Enamine 2 | Enamine | T5225036 | CCOC(═O)C1CCN(CC1)Cn1nc(Nc2ccc(CC)cc2)sc1═S |
| Enamine 2 | Enamine | T5212584 | N#Cc1c(nc(N)c2c(N)nc3N(C)C(═O)Cc3c12)N1CCN(CC1)C(═O)C |
| Enamine 2 | Enamine | T5225046 | Fc1ccc(cc1)n1c(nn(CN2CC(C)OC(C)C2)c1═S)c1cccc(c1)S(═O) |
| | | | (═O)N(C)C |
| Enamine 2 | Enamine | T0512-8800 | c1ccc(nc1)c1nc2ccccc2c(c1)clocnn1 |
| Enamine 2 | Enamine | T5226260 | O═C1CCCCC1Sc1nnc(c2ccco2)n1Cc1ccccc1 |
| Enamine 2 | Enamine | T0513-1160 | CCOC(═O)\C═C1\CC/C(═C/1\N1CCOCC1)/C═N\Nc1ccccc1 |
| Enamine 2 | Enamine | T0520-2213 | CNC(═S)N/N═C/c1ccc(cc1)C(═O)OC |
| Enamine 2 | Enamine | T0512-7666 | O═S(═O)(N1CCCCC1)c1cccc(c1)c1nn2c(nnc2c2ccncc2)s1 |
| Enamine 2 | Enamine | T5227003 | CCCCn1c(═O)[nH]c(═O)c(C(═S)NC(═O)C2CC2)c1N |
| Enamine 2 | Enamine | T5211106 | NC(═S)c1ccccn1 |
| Enamine 2 | Enamine | T0520-0454 | O═C(c1ccco1)c1oc2ccccc2c1N |
| Enamine 2 | Enamine | T0512-8635 | O═C(CCC(═O)c1ccc(F)cc1)OC1CCOC1═O |
| Enamine 2 | Enamine | T0512-3583 | COc1ccc(cc1OC)c1nn(CCC(═O)O)cc1C═C1C(═O)NC(═S)NC1═O |
| Enamine 2 | Enamine | T0513-0201 | O═C(CSc1nnc2ccccn12)Nc1cc(cc(c1)C(═O)O)C(═O)O |
| Enamine 2 | Enamine | T0513-1036 | Clc1ccccc1NC1═NCCCS1 |
| Enamine 2 | Enamine | T5213475 | N#C\C(═C(\C)/N)\C(═O)CSc1nnc(Cc2cccc3ccccc23)n1N |
| Enamine 2 | Enamine | T0520-1835 | CNC(═S)N/N═C/c1cc(C)ccc1C |
| Enamine 2 | Enamine | T0519-0635 | Clc1ccc2Oc3c(C═Nc2c1)c(C)nn3c1ccccc1 |
| Enamine 2 | Enamine | T0519-3860 | S═c1[nH]c(nn1CN1CCCC1)c1cccs1 |
| Enamine 2 | Enamine | T0519-3545 | Clc1ccc(cc1)\C═c1/sc2═NCC(C)(C)Cn2c\1═O |
| Enamine 2 | Enamine | T0517-8266 | CCN(CC)S(═O)(═O)c1cccc(c1)c1nnc(SCc2c(C)noc2C)n1CCc1ccccc1 |
| Enamine 2 | Enamine | T0516-4886 | CCOc1ccc(C(═O)Cc2ccc3oc(cc3c2)C(═O)O)c(O)c1 |
| Enamine 2 | Enamine | T0501-8489 | Cc1cc(O)nc(S)n1 |
| Enamine 2 | Enamine | T0515-8710 | N#C\C(═C(\C)/N1CCCC1)\P(═S)(N1CCOCC1)N1CCOCC1 |
| Enamine 2 | Enamine | T0518-3309 | O═C(\C═C/c1ccco1)OC1CCCCC1═O |
| Enamine 2 | Enamine | T0519-3812 | S═c1[nH]c(nn1CN1CCOCC1)c1cccs1 |
| Enamine 2 | Enamine | T0519-3857 | Fc1ccccc1Nc1nn(CN2CCCC2)c(═S)s1 |
| Enamine 2 | Enamine | T0517-6134 | NNC(═O)Cc1nc2ccccc2n1C |
| Enamine 2 | Enamine | T0517-6101 | N#Cc1ccc(cc1)OCC(═O)N1CCN(CC1)c1ccc(O)cc1 |
| Enamine 2 | Enamine | T0519-3870 | CN(Cn1nc([nH]c1═S)c1cccs1)C1CCCCC1 |
| Enamine 2 | Enamine | T0519-3871 | CN(Cn1nc(Nc2ccccc2F)sc1═S)C1CCCCC1 |
| Enamine 2 | Enamine | T0514-9907 | N#C/C(═C\c1ccc2OCCOc2c1)/C(═O)NC1CCCCC1C |
| Enamine 2 | Enamine | T0515-0810 | NC(═S)CCn1ncc2c(N)ncnc12 |
| Enamine 2 | Enamine | T0517-5499 | CCOc1cc(ccc1OCCOc1ccccc1)C(═O)O |
| Enamine 2 | Enamine | T0519-3854 | S═c1n(CN2CCCC2)nc(C2COc3ccccc3O2)n1c1ccccc1 |
| Enamine 2 | Enamine | T0519-3967 | Clc1cc(Cl)c2nn(CN3C(C)CCCC3C)c(═S)n2c1 |
| Enamine 2 | Enamine | T0519-4284 | CC(C)CN(CC(C)C)Cn1nc([nH]c1═S)c1cccs1 |
| Enamine 2 | Enamine | T0515-8620 | CCCn1c(═O)[nH][nH]c1═S |
| Enamine 2 | Enamine | T0515-0711 | CCOC(═O)c1ccc(cc1)S(═O)(═O)NNS(═O)(═O)c1ccc(C)cc1 |
| Enamine 2 | Enamine | T0517-5511 | N#CCCn1c(S)nc2ccccc12 |
| Enamine 2 | Enamine | T0519-3856 | C═CCN(CC═C)Cn1nc(Nc2ccccc2F)sc1═S |
| Enamine 2 | Enamine | T0517-1513 | Cc1nc(NNS(═O)(═O)c2ccc3ccccc3c2)nc(O)c1 |
| Enamine 2 | Enamine | T5439358 | O═C(CSc1n[nH]/c(═C2\C═c3ccccc3═N/2)/n1c1ccccc1) |
| | | | N1CCNC1═O |
| Enamine 2 | Enamine | T5504596 | CNC(═O)CN(C)Cc1c(O)c(cc2ccccc12)C(═O)NCc1ccccc1 |
| Enamine 2 | Enamine | T5504715 | COC(═O)c1c(C)[nH]c(C(═O)C(C)N2CCN(CC2)c2ccc(O)cc2)c1C |
| Enamine 2 | Enamine | T5439819 | N═c1nc(SCC(═O)c2cc(C)n(C3CCS(═O)(═O)C3)c2C)[nH]c(N)n1 |
| Enamine 2 | Enamine | T5505110 | N═c1sccn1CC(═O)c1cc(C)n(C2CC2)c1C |
| Enamine 2 | Enamine | T5425898 | N#CC(═Cc1cc2OCOc2cc1Br)C#N |
| Enamine 2 | Enamine | T5445942 | CCOc1ccccc1OCc1n[nH]c(═S)n1N |
| Enamine 2 | Enamine | T5252419 | CC1CCCN(C1)C(═O)COC(═O)C1═NN(C(═O)CC1)c1ccccc1 |
| Enamine 2 | Enamine | T5529935 | O═C(NC1CCCCC1C)Cc1sc(═S)[nH]c1C |
| Enamine 2 | Enamine | T5518077 | N#Cc1c(C)cc(C)n(CN2CCN(CC2)C(═O)c2cccs2)c1═S |
| Enamine 2 | Enamine | T5253568 | CCN(CC)C(═O)COc1ccc(cc1)/C(═N\NC(═O)c1ccccc1O)\C |
| Enamine 2 | Enamine | T5243708 | Cn1c(nnc1S)Cc1ccccc1 |
| Enamine 2 | Enamine | T5529934 | O═C(Cc1sc(═S)[nH]c1C)N(C)Cc1ccccc1Cl |
| Enamine 2 | Enamine | T5517626 | N#Cc1cccc(c1)C(═O)OCc1cc(═O)oc2cc(O)ccc12 |
| Enamine 2 | Enamine | T5243711 | CCCc1nnc(S)s1 |
| Enamine 2 | Enamine | T5245853 | COc1cc(/C═N\NS(═O)(═O)c2cc(C)ccc2C)cc(OC)c1O |
| Enamine 2 | Enamine | T0518-2859 | Oc1ccc(cc1)N1CCN(CC1)C(═O)c1ccc(I)cc1 |
| Enamine 2 | Enamine | T0518-4954 | C═CCn1c(S)nnc1C1COc2ccccc2O1 |
| Enamine 2 | Enamine | T0518-6809 | CC1CCCC(C)N1Cn1nc2sc3ccccc3n2c1═S |
| Enamine 2 | Enamine | T0518-0746 | CCS(═O)(═O)c1ccc(O)c(c1)n1c(═S)[nH]c2ccccc2c1═O |
| Enamine 2 | Enamine | T0505-8531 | O═C(OCC(═O)c1ccco1)CSc1cc(C)ccc1C |
| Enamine 2 | Enamine | T0518-6164 | Cc1cc2nn(CN3CCOCC3)c(═S)n2c2ccccc12 |
| Enamine 2 | Enamine | T0507-2701 | O1CCN(CC1)Sc1nc2ccccc2s1 |
| Enamine 2 | Enamine | T0518-7519 | CN(C)CC(C)(C)Cn1c(═S)[H]c2ccccc2c1═O |
| Enamine 2 | Enamine | T0507-2339 | CC(═O)Nc1ccc(cc1)n1cc([nH]c1═S)c1ccccc1 |
| Enamine 2 | Enamine | T5215297 | NC(═S)NN1C(═O)C2CC═CCC2C1═O |
| Enamine 2 | Enamine | T0506-4377 | OC(═O)Cc1ccc(s1)S(═O)(═O)N1CCOCC1 |
| Enamine 2 | Enamine | T0513-6935 | O═C(NN1C(═O)CSC1═S)CN1C(═O)c2ccccc2C1═O |
| Enamine 2 | Enamine | T0518-7383 | N#CCCN(Cc1cccnc1)Cn1nc(c2cccc(c2)S(═O)(═O)N(CC)CC)n |
| | | | (CC(C)C)c1═S |
| Enamine 2 | Enamine | T0518-0705 | N#CCSCc1ccco1 |
| Enamine 2 | Enamine | T0518-6143 | Clc1cc(Cl)c2nn(CN(C)C3CCCCC3)c(═S)n2c1 |
| Enamine 2 | Enamine | T0518-3010 | Cc1ccc2OCCCC(═O)c2c1 |
| Enamine 2 | Enamine | T0506-5702 | O═C(N/N═C/c1ccc(O)c(O)c1O)c1cccc(c1)S(═O)(═O)Nc1ccccc1Cl |
| Enamine 2 | Enamine | T0507-3405 | Cc1nc(C)c(cc1NC(═O)SCC(═O)O)C(═O)OCC |
| Enamine 2 | Enamine | T0506-1739 | O═C(N/N═C/C═C1\N(C)c2ccccc2C\1(C)C)Cc1cn2ccsc2n1 |
| Enamine 2 | Enamine | T0506-6275 | O═C(NN═C1CCC(CC1)C(C)(C)C)Cc1nc2sccn2c1 |
| Enamine 2 | Enamine | T0506-3957 | COCCC/N═C(\c1ccco1)/n1c(═S)nc(c2ccco2)n(CCCOC)c1═S |
| Enamine 2 | Enamine | T5321327 | CCN(CC1COc2ccccc2O1)C(═O)c1ccccc1Cl |
| Enamine 2 | Enamine | T0506-5804 | Cc1ccc(C(═O)N/N═C/c2ccc(O)c(O)c2O)c(C)c1 |
| Enamine 2 | Enamine | T5319291 | CC(═C)CSc1nnc(S)s1 |
| Enamine 2 | Enamine | T0516-6820 | CC1CCCC(C)N1Cn1nc(c2ccccc2)n(c2ccccc2)c1═S |
| Enamine 2 | Enamine | T0516-8413 | COc1ccc(O)c(c1)C(═O)c1cc(C#N)c(═O)n(c1)C(C)C |
| Enamine 2 | Enamine | T0506-2343 | COc1cc(ccc1OC)\C═C1\SC(═O)N(C/1═O)c1c(C)n(C)n(c2ccccc2) |
| | | | c1═O |
| Enamine 2 | Enamine | T0507-1851 | NC(═S)Cc1nnc(N2CCOCC2)n1c1ccccc1 |
| Enamine 2 | Enamine | T0506-4329 | c1coc(c1)c1nc(c2ccco2)c(nc1c1ccco1)c1ccco1 |
| Enamine 2 | Enamine | T5319264 | S═c1[nH]c2ccccc2c(═S)[nH]1 |
| Enamine 2 | Enamine | T0516-7063 | O═C(CSc1nccc(O)n1)CSc1nccc(O)n1 |
| Enamine 2 | Enamine | T5319213 | CCS(═O)(═O)c1ccc2oc(Nc3ccc(cc3)C(C)C)nc2c1 |
| Enamine 2 | Enamine | T0506-3567 | NC(═S)Cc1nn2Cc3ccccc3c2n1 |
| Enamine 2 | Enamine | T0514-2793 | S═C(NNc1nc2ccccc2[nH]1)Nc1ccc(cc1)OC(F)F |
| Enamine 2 | Enamine | T0513-3224 | S═C(NCCc1ccccc1)NNc1nc2ccccc2o1 |
| Enamine 2 | Enamine | T0513-3087 | Fc1ccc(cc1)C(═O)CSCc1ccco1 |
| Enamine 2 | Enamine | T0513-6692 | S═c1[nH]cnc2sccc12 |
| Enamine 2 | Enamine | T0513-3325 | CCN(CC)S(═O)(═O)c1cccc(c1)n1sc2ccccc2c1═O |
| Enamine 2 | Enamine | T0505-3376 | Brc1ccc(O)c(c1)C(═O)N/N═C/c1c(C)n(C)c(═O)n(C)c1═O |
| Enamine 2 | Enamine | T0517-3913 | Cc1nc2sccn2c1C(═S)NC(═O)CC |
| Enamine 2 | Enamine | T0514-5344 | OC1CCCN(C1)Cn1nc(c2ccccc2)n(c2ccc(F)cc2F)c1═S |
| Enamine 2 | Enamine | T0514-7250 | OCC1CCCCN1C(═S)NC(═O)C12CC3CC(CC(C3)C2)C1 |
| Enamine 2 | Enamine | T0517-7965 | CC1CCCN(C1)C(═S)NC(═O)C1CC1 |
| Enamine 2 | Enamine | T0512-4735 | Clc1cc(cc(Br)c1O)NS(═O)(═O)C |
| Enamine 2 | Enamine | T0514-1506 | OC(═O)Cc1ccc(s1)S(═O)(═O)N1CCCC1 |
| Enamine 2 | Enamine | T0514-7258 | N#Cc1ccccc1NC(═S)NC(═O)c1cc(nc2ccccc12)c1ccco1 |
| Enamine 2 | Enamine | T0513-3388 | COc1ccccc1OC(═O)NC(═O)C(F)(F)F |
| Enamine 2 | Enamine | T0514-5358 | CCCN(CC1CC1)Cn1c(═S)sc2ccccc12 |
| Enamine 2 | Enamine | T0517-3957 | O═C(CSc1[nH]c2ccccc2n1)CSc1[nH]c2ccccc2n1 |
| Enamine 2 | Enamine | T0515-4525 | O═C1c2ccccc2C(═O)N1CCOC(═O)c1cc(nc2ccccc12)c1ccco1 |
| Enamine 2 | Enamine | T0512-5707 | S═C(NC(═O)C1CC1)Nc1cccc2c(O)cccc12 |
| Enamine 2 | Enamine | T0517-2799 | COc1ccc(cc1O)\C═C1\Cc2ccccc2C/1═O |
| Enamine 2 | Enamine | T0501-4743 | S═c1[nH]nc2ccccn12 |
| Enamine 2 | Enamine | T0517-5191 | O═C1NNC(═S)C1C1CC1 |
| Enamine 2 | Enamine | T0512-6506 | O═c1[nH]nc(N2CCN(CC2)C(═O)/C═C\c2ccc3OCOc3c2)c(═O) |
| | | | [nH]1 |
| Enamine 2 | Enamine | T0510-3828 | NC(═S)N/N═C(\c1ccccn1)/C(═O)c1ccccn1 |
| Enamine 2 | Enamine | T0515-5890 | CC(═O)Nc1ccc(cc1)NC(═O)C(C)OC(═O)c1cc(nc2ccccc12)c1ccco1 |
| Enamine 2 | Enamine | T0509-6906 | COc1ccc(cc1)c1nn(CN2CCOCC2)c(═S)n1C1CCCCC1 |
| Enamine 2 | Enamine | T0515-1706 | O═C1C\C(═N\Nc2nc3ccccc3s2)\CC(C)(C)C1 |
| Enamine 2 | Enamine | T0509-6936 | COCC(C)n1c(COc2ccccc2F)nnc1S |
| Enamine 2 | Enamine | T0512-7306 | O═C(CSc1oc2ccc(cc2n1)S(═O)(═O)N1CCOCC1)N(C)C |
| Enamine 2 | Enamine | T0509-6938 | C═CCn1c(═O)c2ccccc2n2c(S)nnc12 |
| Enamine 2 | Enamine | T0510-3476 | Cc1ccc(cc1)C(═O)n1ncn(C)c1═S |
| Enamine 2 | Enamine | T0507-8198 | O═C(Nc1sc2CCCCc2c1Sc1nnnn1c1ccccc1)c1ccccc1 |
| Enamine 2 | Enamine | T5306344 | N#Cc1c(N)nc2N(c3ccccc3)C(═O)Cc2c1N |
| Enamine 2 | Enamine | T0515-8103 | CC1CC(C)CN(C1)S(═O)(═O)c1ccc(Cl)c(c1)C(═O)O |
| Enamine 2 | Enamine | T0507-8091 | CC(\C═N\Nc1nc2ccccc2[nH]1)c1ccccc1 |
| Enamine 2 | Enamine | T5214676 | Cc1ccc(O)c(c1)C(═O)c1cnc2nc3ccccc3n2c1 |
| Enamine 2 | Enamine | T5214589 | OCCNC(═O)CCc1nc2ccccc2n1c1ccccc1 |
| Enamine 2 | Enamine | T0516-6576 | O═C(N/N═C1\N═C(N)c2ccccc/12)c1cc2c(C)nn(c3ccccc3)c2s1 |
| Enamine 2 | Enamine | T0507-7784 | S═C(Nc1ncccn1)NC(═O)c1cccs1 |
| Enamine 2 | Enamine | T0506-9600 | COc1ccc(cc1)c1c(C)oc2cc(OCC(O)CN3CCC(CC3)C(═O)N)ccc2c1═O |
| Enamine 2 | Enamine | T0507-0218 | CC1CCCN(C1)C(═S)NCc1ccccc1 |
| Enamine 2 | Enamine | T0504-2723 | CCC(CO)Nc1ncnc2sc3CCCCc3c12 |
| Enamine 2 | Enamine | T0515-1889 | Cc1ccc(cc1)n1c(nnc1c1ccccc1)SCc1nnc2CCCn12 |
| Enamine 2 | Enamine | T0518-0331 | COc1ccc(CCNC(═O)C2═NNC(═O)CC2)cc1 |
| Enamine 2 | Enamine | T0516-3523 | O═C(COC(═O)c1cc(═O)c2ccccc2o1)NC1CCCCC1C |
| Enamine 2 | Enamine | T0516-3948 | O═C(NCc1ccccc1)COC(═O)C1CCN(CC1)S(═O)(═O)c1cccs1 |
| Enamine 2 | Enamine | T0518-0337 | COC(═O)CSc1nnc(c2ccccc2)n1Cc1ccc2OCOc2c1 |
| Enamine 2 | Enamine | T5223786 | O═C(CSc1nnc([nH]1)c1cccs1)c1cc(C)n(Cc2ccco2)c1C |
| Enamine 2 | Enamine | T5224439 | Fc1ccc(cc1)S(═O)(═O)NCc1ccccc1 |
| Enamine 2 | Enamine | T0518-3414 | CCNc1nc(NCC)nc2nnc(SCC(═O)c3c[nH]c4ccccc34)n12 |
| Enamine 2 | Enamine | T0516-3968 | O═C(NCc1ccccc1)COC(═O)c1cnccn1 |
| Enamine 2 | Enamine | T5223269 | OC(═O)C(C)NC1═NS(═O)(═O)c2ccccc12 |
| Enamine 2 | Enamine | T0518-1651 | CCOC(═O)c1oc2ccccc2c1COC(═O)Cn1cnnn1 |
| Enamine 2 | Enamine | T0517-4122 | CCC1CCCCN1CC(═O)c1ccco1 |
| Enamine 2 | Enamine | T5223798 | O═C(N/N═C/c1c(C)nn(c2ccccc2)c1N1CCCC1)C1CC1 |
| Enamine 2 | Enamine | T0516-2795 | N#Cc1ccc(cc1)C(═O)OCC(═O)c1cc2ccccc2o1 |
| Enamine 2 | Enamine | T0504-2415 | N#Cc1nc(oc1NCc1ccccc1)c1ccc(Cl)cc1 |
| Enamine 2 | Enamine | T0504-3463 | CN(C)\N═C\C═C1\CCC(═C/1N1CCOCC1)C(═O)Nc1ccccc1 |
| Enamine 2 | Enamine | T0515-7315 | S═C(N/N═C1\CC(═O)CC(C)(C)C\1)Nc1ccc(cc1)S(═O)(═O) |
| | | | N1CCOCC1 |
| Enamine 2 | Enamine | T0516-3860 | COc1ccc(OC)cc1C(═O)COC(═O)c1ccc2ccccc2n1 |
| Enamine 2 | Enamine | T5360467 | N#Cc1ccccc1Cn1c(═O)c2n(cnc2n(Cc2ccccc2)c1═O)Cc1ccccc1 |
| Enamine 2 | Enamine | T5338384 | O═C(N1CCN(CC1)c1ncccn1)C1═NN(C(═O)CC1)c1ccccc1 |
| Enamine 2 | Enamine | T5442076 | O═C(NCCCN1CCOCC1)c1cc2ccccc2cc1O |
| Enamine 2 | Enamine | T5338792 | CC(═O)Nc1ccc(cc1)C(═O)OCC(═O)N1CCCC1═O |
| Enamine 2 | Enamine | T5330110 | O═C(C(C)O/N═C/c1ccco1)N1CCN(CC1)Cc1ccccc1 |
| Enamine 2 | Enamine | T5442114 | CCC(═O)N(C1CC1)c1nnc(SCc2cc(═O)n3c(C)csc3n2)s1 |
| Enamine 2 | Enamine | T5330137 | COc1ccccc1NC(═O)CN(Cc1ccco1)C(═O)c1cccs1 |
| Enamine 2 | Enamine | T5361098 | CCN(CC(═O)Nc1ccc(cc1)NC(═O)C)C(═O)C1CCCC1 |
| Enamine 2 | Enamine | T5338315 | COc1ccccc1NC(═O)Cc1noc(COC(═O)c2cccc(c2)S(═O)(═O) |
| | | | N2CCc3ccccc23)n1 |
| Enamine 2 | Enamine | T5338036 | COc1ccccc1N(C)S(═O)(═O)c1ccc(cc1)C(═O)OCC(═O)N1CCC1 |
| Enamine 2 | Enamine | T5337323 | c1ccc(cc1)c1nc(nnc1c1ccccc1)C1CC1 |
| Enamine 2 | Enamine | T5338062 | Cc1cnc(cn1)C(═O)OCC(═O)c1csc(n1)N1CCCCC1 |
| Enamine 2 | Enamine | T5337159 | NC(═O)COC(═O)c1sc2CCCc2c1 |
| Enamine 2 | Enamine | T5334882 | CCOC(═O)c1cc2c(N)n[nH]c2[nH]c1═O |
| Enamine 2 | Enamine | T0501-2492 | COc1ccc(cc1N)S(═O)(═O)SC |
| Enamine 2 | Enamine | T5347679 | CC(═O)Nc1ccc(cc1)S(═O)(═O)N(Cc1ccco1)S(═O)(═O)c1ccccc1F |
| Enamine 2 | Enamine | T5339020 | Cc1ccc(c(C)c1)N1C(═O)/C(═C\NNc2nc3cc(ccc302)S(═O)(═O) |
| | | | N2CCOCC2)\c2ccccc2C1═O |
| Enamine 2 | Enamine | T5336016 | N#Cc1c(nc(N)c2c(N)nc(SCC(═O)O)cc12)N(C)CCc1ccc(OC)c(OC)c1 |
| Enamine 2 | Enamine | T0501-2496 | O═c1[nH]nc(N/N═C2\CC(═O)CC(C)(C)C\2)c(═O)[nH]1 |
| Enamine 2 | Enamine | T0501-4107 | Cc1cc(C)nc(NN═C2CCCC2)n1 |
| Enamine 2 | Enamine | T0501-6198 | O═C(Nc1nccs1)C1C(C(═O)Nc2nccs2)C1═C |
| Enamine 2 | Enamine | T0500-0137 | CN(N)P(═S)(N(C)N)c1ccccc1 |
| Enamine 2 | Enamine | T0501-8566 | Ic1cc2oc3CCCCc3c2c(/C═N\Cc2ccncc2)c1O |
| Enamine 2 | Enamine | T0501-7343 | COc1ccc(cc1)C(═O)CSc1[nH]c2c(═O)[nH]c(═O)n(C)c2n1 |
| Enamine 2 | Enamine | T5515717 | Cc1cc(NNS(═O)(═O)c2ccc(cc2)OC(F)(F)F)n2ncnc2n1 |
| Enamine 2 | Enamine | T5528041 | S═C(NNc1ccc(cc1)C(═O)O)NC1CC1 |
| Enamine 2 | Enamine | T0518-5673 | N#Cc1cc(cn(CC2CCCO2)c1═O)C(═O)c1cc(OC)ccc1O |
| Enamine 2 | Enamine | T5526469 | CCOC(═O)Nc1ccc2c(CNCC(C)C)cc(═O)oc2c1 |
| Enamine 2 | Enamine | T5232515 | N#Cc1c(NC═C2C(═O)N(C)C(═O)N(C)C2═O)sc2CCCc12 |
| Enamine 2 | Enamine | T5426213 | O═C(OCc1ccco1)CCN1C(═O)c2ccccc2C1═O |
| Enamine 2 | Enamine | T5422614 | N#Cc1cccnc1S |
| Enamine 2 | Enamine | T5422616 | NC(═O)Cc1cc(═O)[nH]c(═S)[nH]1 |
| Enamine 2 | Enamine | T5429778 | OC(═O)CCc1ccc(cc1)S(═O)(═O)Nc1cc(ccc1N1CCCC1)C(F)(F)F |
| Enamine 2 | Enamine | T5306364 | O═c1cc(CSc2nnc(c3ccncc3)n2N)c2cc(O)c(O)cc2o1 |
| Enamine 2 | Enamine | T5386516 | NNC(═O)Cc1[nH]n(c2ccc(F)cc2)c(═O)c1 |
| Enamine 2 | Enamine | T5359487 | NNC(═O)Cc1cc(═O)n(CC(C)C)[nH]1 |
| Enamine 2 | Enamine | T5391565 | Cc1cc(C)nc(NNC═C2C(═O)CC(C)(C)CC2═O)n1 |
| Enamine 2 | Enamine | T5221979 | Cc1ccc(cc1)S(═O)(═O)N1CCN(CC1)Cn1nc(Nc2ccccc2F)sc1═S |
| Enamine 2 | Enamine | T0400-1171 | O═C1C═CC(═O)N1c1ccc2ccccc2c1 |
| Enamine 2 | Enamine | T5301829 | Clc1ccc(cc1)n1ccnc1S |
| Enamine 2 | Enamine | T5222002 | S═c1[nH]c(nn1CN1CCN(CC1)S(═O)(═O)c1ccc2ccccc2c1)c1cccs1 |
| Enamine 2 | Enamine | T5294607 | CCN(CC)C(═O)CSc1nnc(S)s1 |
| Enamine 2 | Enamine | T5303095 | O═C(Nc1ccc(cc1)n1cnnn1)c1cccc(c1)S(═O)(═O)N1CCc2ccccc2C1 |
| Enamine 2 | Enamine | T5504271 | S═c1[nH]c(nn1CN1CCc2ccccc2C1)c1ccccc1 |
| Enamine 2 | Enamine | T5536877 | Cc1ccc(NC(═O)CNC(═O)C23CC4CC(CC(C4)C3)C2)c(O)c1 |
| Enamine 2 | Enamine | T5512248 | O═C(COC(═O)c1cccc(c1)n1cccc1)N1CCCC1═O |
| Enamine 2 | Enamine | T5536108 | O═C(CCc1ccco1)N1CCCC(C1)c1nc2ccccc2s1 |
| Enamine 2 | Enamine | T5536852 | CC(═O)Nc1cc(NC(═O)C)cc(c1)C(═O)N1CCCC2CCCCC12 |
| Enamine 2 | Enamine | T5426952 | OC(═O)c1cc(Cl)c[nH]1 |
| Enamine 2 | Enamine | T5512337 | O═C(NC1(Oc2ccccc2O1)C(F)(F)F)N1CCCC1 |
| Enamine 2 | Enamine | T5423230 | COc1ccc(C)cc1S(═O)(═O)NNc1nc(C)cc(C)n1 |
| Enamine 2 | Enamine | T5499106 | O═C(OCC(═O)c1[nH]ccc1)CN1C(═O)S/C(═C\c2cccs2)/C1═O |
| Enamine 2 | Enamine | T5415173 | O═C1CCCCC1OC(═O)c1ccc2C(═O)N(Cc3ccco3)C(═O)c2c1 |
| Enamine 2 | Enamine | T5520038 | COc1ccc(CCNC(═S)N2CCCC(C2)C(F)(F)F)cc1 |
| Enamine 2 | Enamine | T5519492 | Cccccc(NNC(═O)C2CCCCC2)c1 |
| Enamine 2 | Enamine | T5519571 | FC(F)(F)C1CCCN(C1)c1nsc2ccccc12 |
| Enamine 2 | Enamine | T5534227 | S═c1[nH]cnc2ccsc12 |
| Enamine 2 | Enamine | T5495369 | CNc1scc(n1)c1ccc(O)cc1O |
| Enamine 2 | Enamine | T5538901 | O═C(COCc1nc2ccccc2s1)Nc1ccc(cc1)c1nnc2CCCCCn12 |
| Enamine 2 | Enamine | T5305318 | CNC(═O)c1c(C)nc(S)c(C#N)c1c1ccco1 |
| Enamine 2 | Enamine | T5305326 | CNc1nc(═S)[nH]c2ccccc12 |
| Enamine 2 | Enamine | T5307301 | Cc1cc(C)c(C)c(S(═O)O)c1C |
| Enamine 2 | Enamine | T5343152 | Cc1scc(COC(═O)c2cc(O)c3ccccc3c2O)n1 |
| Enamine 2 | Enamine | T5305343 | Sc1nnc(NCCCN2CCOCC2)s1 |
| Enamine 2 | Enamine | T5342871 | NC(═S)CC(═S)N1CCOCC1 |
| Enamine 2 | Enamine | T5343010 | Sc1nnc(NC2CC2)s1 |
| Enamine 2 | Enamine | T5353580 | N#CC1C(═O)NC(═S)C(C(═O)N)C21CCCCC2 |
| Enamine 2 | Enamine | T5350560 | COc1ccc(cc1)S(═O)(═O)n1c(═O)cnc2ccccc12 |
| Enamine 2 | Enamine | T5482290 | N#C\C(═C(\C)/N)\C(═O)CSc1nnc2c(Cl)cc(Cl)cn12 |
| Enamine 2 | Enamine | T5474303 | COc1ccc(cc1)c1n[nH]c(═S)[nH]c1═O |
| Enamine 2 | Enamine | T0515-4592 | CC(═C)C/N═c1/scc(c2cccs2)n/1\N═C\c1ccc(O)c(O)c1 |
| Enamine 2 | Enamine | T0512-3788 | Sc1nnc(C(C)C)n1N |
| Enamine 2 | Enamine | T0512-2275 | Oc1nc2nnc(S)n2nc1C |
| Enamine 2 | Enamine | T0512-8383 | S═C(NNc1ccc(C)cc1)NC1CCCCC1 |
| Enamine 2 | Enamine | T0501-3854 | NNC(═S)\N═C/c1ccco1 |
| Enamine 2 | Enamine | T0510-7562 | Brc1cccc(/C═N\NC(═O)c2ccccc2O)c1 |
| Enamine 2 | Enamine | T0514-5126 | CCc1sc2ncnc(S)c2c1 |
| Enamine 2 | Enamine | T0510-6731 | O═c1oc2ccccc2[nH]1 |
| Enamine 2 | Enamine | T0517-2361 | CCOc1ccc(cc1)S(═O)(═O)N═C1C═CC(═O)C═C1 |
| Enamine 2 | Enamine | T0515-1927 | Oc1c(ccc2cccnc12)C(N1CCCCC1)c1cccs1 |
| Enamine 2 | Enamine | T5474551 | Oc1c(ccc2cccnc12)CN1CCCCC1 |
| Enamine 2 | Enamine | T5378965 | OC(═O)C(C)n1c(S)nnc1c1cccs1 |
| Enamine 2 | Enamine | T5473232 | COc1cccc(c1)C(Nc1ccccn1)c1oc(CO)cc(═O)c1O |
| Enamine 2 | Enamine | T0516-1631 | Cn1c(S)nc2sccc2c1═O |
| Enamine 2 | Enamine | T5224658 | OC(═O)CCSc1nnc(S)s1 |
| Enamine 2 | Enamine | T5229935 | CC(═O)Nc1ccc(cc1)S(═O)(═O)Nc1nc2ccccc2nc1Nc1ccc(C(═O) |
| | | | O)c(O)c1 |
| Enamine 2 | Enamine | T5365031 | OC(═O)\C═C/c1cccn1C |
| Enamine 2 | Enamine | T5298840 | N#Cc1c(C)cc(═O)[nH]c1S |
| Enamine 2 | Enamine | T0518-0602 | C1CCC2(CC1)Oc1ccccc1O2 |
| Enamine 2 | Enamine | T5371551 | O═C1CN(\N═C\c2ccc(o2)c2ccc(cc2)S(═O)(═O)Nc2ncccn2)C |
| | | | (═C1c1nc2ccccc2s1)N |
| Enamine 2 | Enamine | T0502-8525 | S═C(Nc1ccc(cc1)N(C)C)N═P(N(C)C)(N(C)C)c1ccncc1 |
| Enamine 2 | Enamine | T0506-4306 | Sc1nnc(s1)Nc1ccccc1C |
| Enamine 2 | Enamine | T0502-3042 | CCCN(CCC)C1═CC(═O)C(═C(C)C1═O)C |
| Enamine 2 | Enamine | T0508-4734 | O═C(N/N═C/c1ccc(O)c(O)c1O)c1ccc(CSc2nc3ccccc3o2)cc1 |
| Enamine 2 | Enamine | T5437483 | S═c1[nH]c2ccccc2c(═O)n1c1nnc[nH]1 |
| Enamine 2 | Enamine | T5421512 | N#CC1C(═O)NC(═C(C#N)C1(C)C)S |
| Enamine 2 | Enamine | T5507363 | Cc1scc(CSc2nnc(c3cccc(c3)S(═O)(═O)N(C)C)n2c2cccc(C)c2)n1 |
| Enamine 2 | Enamine | T0510-2176 | COc1cc(OC)c(Cl)cc1NS(═O)(═O)c1ccc(C)c(c1)C(═O)Nc1nnn[nH]1 |
| Enamine 2 | Enamine | T0514-3372 | S═C1NC(═O)C(CNc2ccccc2C)S1 |
| Enamine 2 | Enamine | T5358965 | Fc1ccc(cc1)n1c(nnc1c1ccncc1)SC1CCCC1═O |
| Enamine 2 | Enamine | T0515-9025 | O═C(COC(═O)c1cc(O)c(O)c(O)c1)NC1CCCc2ccccc12 |
| Enamine 2 | Enamine | T0515-7154 | COCCn1c(NC(═S)NC(═O)C2CC2)cc(═O)[nH]c1═O |
| Enamine 2 | Enamine | T0508-4535 | O═C(N/N═C/c1ccc(O)c(O)c1O)c1ccccc1n1cccc1 |
| Enamine 2 | Enamine | T0515-8355 | S═C(NNc1ccc(Cl)c(c1)C(═O)O)NC1CCCCC1 |
| Enamine 2 | Enamine | T0509-8494 | Sc1nc2c(═O)nc(S)[nH]c2[nH]1 |
| Enamine 2 | Enamine | T0512-2414 | Sc1nnc([nH]1)c1cccs1 |
| Enamine 2 | Enamine | T5359509 | N#Cc1c(N)cc(N)nc1S |
| Enamine 2 | Enamine | T0515-6184 | N═C1N═C\C(═N/Nc2cc(cc(c2)C(═O)O)C(═O)O)/S1 |
| Enamine 2 | Enamine | T0517-0129 | O═C1CCCC(═O)C1═NNc1ccccc1C(═O)O |
| Enamine 2 | Enamine | T0515-1673 | S═C(Nn1cnnc1)NC(═O)C1CCCCC1 |
| Enamine 2 | Enamine | T0515-7122 | S═C(NC(═O)C1CC1)N1CCN(C(═S)NC(═O)C2CC2)C(C)C1 |
| Enamine 2 | Enamine | T0515-8438 | COc1ccc(cc1)NC(═S)NNc1ccc(cc1)C(═O)O |
| Enamine 2 | Enamine | T0514-3358 | S═C1NC(═O)C(CNc2cccc(c2)C(═O)O)S1 |
| Enamine 2 | Enamine | T0515-9018 | O═C(COC(═O)c1cc(O)c(O)c(O)c1)c1ccc(cc1)C1CCCCC1 |
| NIH Clinical | Tocris | SAM001247031 | Oc1cc(O)c2C[C@@H](OC(═O)c3cc(O)c(O)c(O)c3)[C@H](Oc2c1) |
| Collection 1 - | Cookson | | c4cc(O)c(O)c(O)c4 |
| 2014 | Ltd. | | |
| NIH Clinical | Sequoia | SAM001246818 | Nc1nc(cs1)C(═NO)C(═O)N[C@H]2[C@H]3SCC(═C(N3C2═O)C |
| Collection 1 - | Research | | (═O)O)C═C |
| 2014 | Products | | |
| | Ltd. | | |
| NIH Clinical | Tocris | SAM001247083 | Oc1cc2CC[C@H]3NCc4ccccc4[C@@H]3c2cc1O•O•Cl— |
| Collection 1 - | Cookson | | |
| 2014 | Ltd. | | |
| Biomol 4 - FDA | BIOMOL | EI-165 | c1(CNNC(═O)C(N)CO)ccc(O)c(O)c1O |
| Approved Drug | | | |
| Library | | | |
| Biomol 4 - FDA | BIOMOL | DL-106 | C(SSC1═S)(═C1C)c2nccnc2 |
| Approved Drug | | | |
| Library | | | |
| Biomol 4 - FDA | BIOMOL | DL-348 | N(C(C([O—])═O)═C(C[n+]1ccccc1)CS2)(C(═O)[C@H]3NC(═O)\ |
| Approved Drug | | | C(═N/OC(C)(C)C(═O)O)\c4nc(N)sc4)[C@H]23 |
| Library | | | |
| Enamine 1 | Enamine | T0501-0693 | COc1ccc(cc1)\N═C/c1ccc2ncccc2c1 |
| Enamine 1 | Enamine | T0503-3218 | O═c1c2cccc3cccc(c23)n1S(═O)(═O)c1cccs1 |
| Enamine 1 | Enamine | T0505-2004 | CCOc1ccc(cc1)NC1═CC(═O)C═CC1═O |
| ChemDiv1 | ChemDiv | 1464-0277 | COc1cc(ccc1OCc1ccc(cc1)[N+](═O)[O—])/ |
| (Combilab and | | | C═C1\SC(═S)N(CC(═O)O)C/1═O |
| International) | | | |
| ChemDiv1 | ChemDiv | 1545-0256 | OCCNc1cc(F)nc(N)n1 |
| (Combilab and | | | |
| International) | | | |
| ChemDiv1 | ChemDiv | 1630-1506 | CCOCCOC(═O)C1═C(C)NC2═C(C(═O)CC(C)(C)C2)C1c1cc(Br)c(O) |
| (Combilab and | | | c(OC)c1 |
| International) | | | |
| ChemDiv1 | ChemDiv | 1630-1729 | O═C(OCc1ccccc1)C1═C(C)N(C)C(═O)NC1c1ccc(OCc2ccccc2)cc1 |
| (Combilab and | | | |
| International) | | | |
| ChemDiv1 | ChemDiv | 1611-4804 | CCOC(═O)C1═C(C)N═c2s/c(═C/c3cc(Br)ccc3OC(═O)C)/c(═O) |
| (Combilab and | | | n2C1c1ccccc1 |
| International) | | | |
| ChemDiv1 | ChemDiv | 1852-0310 | CCOC(═O)C1═C(C)OC(═C(C#N)C1c1cccs1)N |
| (Combilab and | | | |
| International) | | | |
| ChemDiv1 | ChemDiv | 1927-7855 | CCSc1nnc2c(n1)OC(Nc1ccccc21)c1ccc(o1)[N+](═O)[O—] |
| (Combilab and | | | |
| International) | | | |
| ChemDiv1 | ChemDiv | 2155-0006 | CC(═O)c1ccc2NC(C3CC═CC3c2c1)c1cccc2ccccc12 |
| (Combilab and | | | |
| International) | | | |
| Enamine 1 | Enamine | T0506-1917 | Clc1ccc(cc1)SCC(═O)c1ccco1 |
| Enamine 1 | Enamine | T0507-5780 | NNc1ccccc1C(F)(F)F |
| Enamine 1 | Enamine | T0508-7813 | Oc1ccc(cc1)N1CCN(CC1)C(═O)CSc1ccc2ccccc2c1 |
| Enamine 1 | Enamine | T0510-1734 | c1coc(c1)c1cn2c(n1)sc1ccccc21 |
| Enamine 1 | Enamine | T0510-3387 | Cc1ccc(C)n1CCN1CCN(CC1)S(═O)(═O)c1ccccc1 |
| Enamine 1 | Enamine | T0510-7914 | OC(═O)c1nc2cccc3cccc([nH]1)c23 |
| Enamine 1 | Enamine | T0511-7669 | O═C(CSCc1ccco1)c1ccccc1F |
| Enamine 1 | Enamine | T0514-0118 | OC(═O)\C═C/C(═O)c1ccc(F)cc1 |
| Enamine 1 | Enamine | T0514-5241 | COc1ccc(cc1)CCN1C(═O)C═CC1═O |
| ChemDiv | ChemDiv | C066-3867 | c1(CSc(c23)cccc2)c3[nH]nc1C(NCCC4═CCCCC4)═O |
| Targeted | | | |
| Diversity Library | | | |
| ChemDiv | ChemDiv | C200-4690 | C12═C(SC(═S)N1c3cccc(OC)c3)C(N4C(c5c(cccc5)C(N4)═O)═N2)═O |
| Targeted | | | |
| Diversity Library | | | |
| ChemDiv | ChemDiv | C200-2668 | C(C1)(═C(CCN1C(C)c2ccccc2)NC(N3)═S)C3═O |
| Targeted | | | |
| Diversity Library | | | |
| ChemDiv | ChemDiv | C200-7834 | C1(═NNC2═S)N2c(c3C(═O)N1CC4CCCO4)ccs3 |
| Targeted | | | |
| Diversity Library | | | |
| ChemDiv | ChemDiv | C200-7283 | C1(═NNC2═S)N2c(cccc3)c3C(═O)N1Cc(cc4)ccc4C |
| Targeted | | | |
| Diversity Library | | | |
| ChemDiv | ChemDiv | C200-7014 | C1(═NNC2═S)N2c(c3C(═O)N1CCCC)ccs3 |
| Targeted | | | |
| Diversity Library | | | |
| ChemDiv | ChemDiv | C200-7326 | C1(═NNC2═S)N2c(c3C(═O)N1CCC(C)C)cccc3 |
| Targeted | | | |
| Diversity Library | | | |
| ChemDiv | ChemDiv | C200-8090 | C1(═NNC2═S)N2c3c(cc(cc3)F)C(═O)N1CCC |
| Targeted | | | |
| Diversity Library | | | |
| ChemDiv | ChemDiv | C200-7093 | C1(═NNC2═S)N2c(c3C(═O)N1CC(C)C)ccs3 |
| Targeted | | | |
| Diversity Library | | | |
| ChemDiv | ChemDiv | C200-7327 | C1(═NNC2═S)N2c(cccc3)c3C(═O)N1CC(C)C |
| Targeted | | | |
| Diversity Library | | | |
| ChemDiv | ChemDiv | C200-7329 | C1(═NNC2═S)N2c3c(C(═O)N1CC(C)C)ccc(c3)C(═O)NC4CCCC4 |
| Targeted | | | |
| Diversity Library | | | |
| ChemDiv | ChemDiv | C200-7463 | c1(C2═O)c(c(nn1CC)C)NC(═S)N2CC3CCCO3 |
| Targeted | | | |
| Diversity Library | | | |
| ChemDiv | ChemDiv | C200-8885 | C1(═NNC2═S)N2c3c(cc(cc3)Cl)C(═O)N1CCC |
| Targeted | | | |
| Diversity Library | | | |
| ChemDiv | ChemDiv | C200-9422 | C1(═NNC2═S)N2c(c3C(═O)N1CCC(═O)NC4CCCCC4)cccc3 |
| Targeted | | | |
| Diversity Library | | | |
| ChemDiv | ChemDiv | C200-9423 | C1(═NNC2═S)N2c(c3C(═O)N1CCC(═O)NC(C)CC)cccc3 |
| Targeted | | | |
| Diversity Library | | | |
| ChemDiv | ChemDiv | C200-9425 | C1(═NNC2═S)N2c3c(cc(cc3)Cl)C(═O)N1CCC(═O)NC(C)C |
| Targeted | | | |
| Diversity Library | | | |
| ChemDiv | ChemDiv | C201-1864 | c(sc(n1)N(CC)CC)(C2═O)c1NC(═S)N2C(C)C |
| Targeted | | | |
| Diversity Library | | | |
| ChemDiv | ChemDiv | C202-1816 | c12c(n[nH]c1c3ccc(cc3)F)nc(c4ccc(c(O)c4)O)cc2C(O)═O |
| Targeted | | | |
| Diversity Library | | | |
| ChemDiv | ChemDiv | C243-0026 | c1(C2═O)c(sc(C(═O)Nc3cccc(C)c3)c1)N═C(C═CC═C4)N24 |
| Targeted | | | |
| Diversity Library | | | |
| ChemDiv | ChemDiv | C301-8945 | n12c(c3c(cccc3)c(NCCCc4ccccc4)n1)nnn2 |
| Targeted | | | |
| Diversity Library | | | |
| ChemDiv | ChemDiv | C301-7136 | n1(nc(n2)c3ccc(cc3)C)c2nc(C)cc1Nc4c(OC)cc(c(Cl)c4)OC |
| Targeted | | | |
| Diversity Library | | | |
| ChemDiv | ChemDiv | C301-9367 | C1(═O)N(C)c(c2N1C)ccc(c2)S(═O)(═O)c(ccc(c3N4C)N(C4═O)C)c3 |
| Targeted | | | |
| Diversity Library | | | |
| ChemDiv | ChemDiv | C301-9375 | c12c(c(nc(Nc(ccc(c3Cl)OC)c3)n1)C)nc(CC)n2c4ccc(c(Cl)c4)OC |
| Targeted | | | |
| Diversity Library | | | |
| ChemDiv | ChemDiv | C301-8999 | n1(ncn2)c2nc(c(CC)c1Sc3ccccc3NC(═O)Nc4ccc(c(OC)c4)OC)C |
| Targeted | | | |
| Diversity Library | | | |
| ChemDiv | ChemDiv | C594-0003 | n1(C(c(cc2)ccn2)═O)nc(c(cc(ccc(OC)c3)c3n4)c14)N |
| Targeted | | | |
| Diversity Library | | | |
| ChemDiv | ChemDiv | C594-0010 | n1(C(═O)c2ccc(cc2)F)nc(c(cc(ccc(OC)c3)c3n4)c14)N |
| Targeted | | | |
| Diversity Library | | | |
| ChemDiv | ChemDiv | C742-0312 | n12c(ccc(SCC(═O)Nc3nnc(C)s3)n1)nnc2c4ccc(cc4)F |
| Targeted | | | |
| Diversity Library | | | |
| ChemDiv | ChemDiv | C770-0245 | S(═O)(═O)(c1ccc(cc1)F)c2c3c(ccc(F)c3)ncc2C(c4ccccc4)═O |
| Targeted | | | |
| Diversity Library | | | |
| ChemDiv | ChemDiv | C793-0254 | c1(C(OC)═O)oc(c2c1O)cccc2 |
| Targeted | | | |
| Diversity Library | | | |
| ChemDiv | ChemDiv | D087-0519 | S(═O)(═O)(Nc1c(C)ccc(Cl)c1)C(CCS2(═O)═O)C2 |
| Targeted | | | |
| Diversity Library | | | |
| ChemDiv | ChemDiv | D132-0053 | c12c(C(═O)C═C(c3ccc(c(OC)c3)OC)C═C1OC(═O)c4ccc(cc4)OC) |
| Targeted | | | c(oc2C)C |
| Diversity Library | | | |
| ChemDiv | ChemDiv | D212-0373 | c1(C(CC(N2CC(C)C)═O)C2)n(C)c3c(cccc3)n1 |
| Targeted | | | |
| Diversity Library | | | |
| ChemDiv | ChemDiv | D226-0165 | c12c(C(NC(═O)N1C)═O)n(c(SCC(CO)O)n2)Cc3cccc(C)c3 |
| Targeted | | | |
| Diversity Library | | | |
| ChemDiv | ChemDiv | D278-0547 | c1(Nc(cc2)ccc2N(CC3)CCO3)cc(c4ccccc4)nc(C)n1 |
| Targeted | | | |
| Diversity Library | | | |
| ChemDiv | ChemDiv | D316-0527 | S1(═O)(═O)c(cccc2)c2C(═C(C(OC)═O)N1C)OC(═O)CN3C(═O) |
| Targeted | | | c(c4C3═O)cccc4 |
| Diversity Library | | | |
| ChemDiv | ChemDiv | D305-1386 | c1(CN2CCCCC2)n(C(C)C)c(c3n1)ccc(c3)NC(C)═O |
| Targeted | | | |
| Diversity Library | | | |
| ChemDiv | ChemDiv | D344-7204 | c1(c2ccccc2F)oc(c3n1)ccc(c3)NC(COc(cc4)ccc4F)═O |
| Targeted | | | |
| Diversity Library | | | |
| ChemDiv | ChemDiv | D398-0910 | N1(CCCC1c2ccccc2F)C(═O)c(cccn3)c3 |
| Targeted | | | |
| Diversity Library | | | |
| ChemDiv | ChemDiv | D513-3628 | c(N(CCOc1ccccc1)S(c2ccccc2)(═O)═O)(nn3c4nc(c(Cl)c3C)C)n4 |
| Targeted | | | |
| Diversity Library | | | |
| ChemDiv | ChemDiv | D585-0166 | S1(CCC(C1)NC(CSc2nc(O)c3c([nH]cn3)n2)═O)(═O)═O |
| Targeted | | | |
| Diversity Library | | | |
| ChemDiv | ChemDiv | D664-0047 | C1(═O)N(C)c2c(cc(cc2)CN([H])c3nnnn3CCCC)N1C |
| Targeted | | | |
| Diversity Library | | | |
| ChemDiv | ChemDiv | D686-0195 | c1(NC(═O)c2ccc(nc2Cl)C)sc(nc1C(N)═O)Nc3cc(C)ccc3C |
| Targeted | | | |
| Diversity Library | | | |
| ChemDiv | ChemDiv | D686-0236 | c1(NC(═O)c2ccc(cc2)F)sc(nc1C(N)═O)Nc3cccc(C)c3C |
| Targeted | | | |
| Diversity Library | | | |
| ChemDiv | ChemDiv | D715-0997 | n1n[nH]c(COc(ccc(c23)C═CC(═O)O2)c3)n1 |
| Targeted | | | |
| Diversity Library | | | |
| ChemDiv | ChemDiv | D727-0113 | n1(nc(s2)COc(ccc(c3C)C)c3)c2nnc1c4ccoc4C |
| Targeted | | | |
| Diversity Library | | | |
| ChemDiv | ChemDiv | D727-0215 | n12c(nnc1CSc3ccccc3)sc(c4cccc(N(C)C)c4)n2 |
| Targeted | | | |
| Diversity Library | | | |
| ChemDiv | ChemDiv | D727-0612 | n12c(nnc1C(C)C)sc(c3cc(on3)C)n2 |
| Targeted | | | |
| Diversity Library | | | |
| ChemDiv | ChemDiv | D727-0714 | n1(c(CCc(c(C)nn2c3ccccc3)c2C)nn4)c4sc(c5ccc(cc5)F)n1 |
| Targeted | | | |
| Diversity Library | | | |
| ChemDiv | ChemDiv | D727-0115 | n1(nc(s2)COc(cc3)ccc3Cl)c2nnc1c4ccoc4C |
| Targeted | | | |
| Diversity Library | | | |
| ChemDiv | ChemDiv | D727-0216 | n1(nc(s2)Cc(ccc(c34)OCCO3)c4)c2nnc1CSc5ccccc5 |
| Targeted | | | |
| Diversity Library | | | |
| ChemDiv | ChemDiv | D727-0339 | n12c(SCC(N)═N1)nnc2Cn(c3c(n4)cccc3)c4C |
| Targeted | | | |
| Diversity Library | | | |
| ChemDiv | ChemDiv | D727-0116 | n1(nc(s2)COc3ccccc3Cl)c2nnc1c4ccoc4C |
| Targeted | | | |
| Diversity Library | | | |
| ChemDiv | ChemDiv | D727-0342 | n12c(SCC(c3ccc(cc3)Cl)═N1)nnc2CCOC |
| Targeted | | | |
| Diversity Library | | | |
| ChemDiv | ChemDiv | D727-0446 | n12c(nnc1COc(cc3)ccc3Br)sc(c4ccc(c(OC)c4)OC)n2 |
| Targeted | | | |
| Diversity Library | | | |
| ChemDiv | ChemDiv | D727-0071 | n1(nc(s2)CCc3ccccc3)c2nnc1c(cccn4)c4 |
| Targeted | | | |
| Diversity Library | | | |
| ChemDiv | ChemDiv | D727-0117 | n1(nc(s2)COc(cc3)ccc3F)c2nnc1c4ccoc4C |
| Targeted | | | |
| Diversity Library | | | |
| ChemDiv | ChemDiv | D727-0210 | n1(nc(s2)COc3ccccc3OC)c2nnc1CSc4ccccc4 |
| Targeted | | | |
| Diversity Library | | | |
| ChemDiv | ChemDiv | D727-0746 | n1(nc(s2)Cc(ccc(c3OC)OC)c3)c2nnc1c4ccc(c5n4)cccc5 |
| Targeted | | | |
| Diversity Library | | | |
| ChemDiv | ChemDiv | D727-0191 | n12c(nnc1CSc3ccccc3)sc(c4ccccc4OC)n2 |
| Targeted | | | |
| Diversity Library | | | |
| ChemDiv | ChemDiv | D727-0200 | n1(nc(s2)Cc(ccc(c3OC)OC)c3)c2nnc1CSc4ccccc4 |
| Targeted | | | |
| Diversity Library | | | |
| ChemDiv | ChemDiv | D727-0824 | n1(nc(s2)CCc(c(C)nn3c4ccccc4)c3C)c2nnc1C(C)(C)C |
| Targeted | | | |
| Diversity Library | | | |
| ChemDiv | ChemDiv | E135-0568 | S(═O)(═O)(N═C(c1ccc(cc1)F)C═C2C(═O)Nc3sc(c4c3C(N)═O) |
| Targeted | | | CCCC4)N2C |
| Diversity Library | | | |
| ChemDiv | ChemDiv | D727-0828 | n1(nc(s2)CCc(c(C)nn3c4ccccc4)c3C)c2nnc1c5cccc(F)c5 |
| Targeted | | | |
| Diversity Library | | | |
| ChemDiv | ChemDiv | D727-0890 | n12c(nnc1c3cccc(Cl)c3)sc(c4ccc(c5n4)cccc5)n2 |
| Targeted | | | |
| Diversity Library | | | |
| ChemDiv | ChemDiv | D727-0910 | n1(nc(s2)COc3ccccc3Cl)c2nnc1c4cc(n[nH]4)C |
| Targeted | | | |
| Diversity Library | | | |
| ChemDiv | ChemDiv | D727-0829 | n1(nc(s2)CCc(c(C)nn3c4ccccc4)c3C)c2nnc1c5ccc(cc5)F |
| Targeted | | | |
| Diversity Library | | | |
| ChemDiv | ChemDiv | D727-0845 | n12c(nnc1C(C)C)sc(c3cc(n[nH]3)CC(C)C)n2 |
| Targeted | | | |
| Diversity Library | | | |
| ChemDiv | ChemDiv | D727-0857 | n12c(nnc1c3ccc(cc3)F)sc(c4cc(n[nH]4)CC(C)C)n2 |
| Targeted | | | |
| Diversity Library | | | |
| ChemDiv | ChemDiv | D727-0911 | n1(nc(s2)COc(cc3)ccc3F)c2nnc1c4cc(n[nH]4)C |
| Targeted | | | |
| Diversity Library | | | |
| ChemDiv | ChemDiv | D727-0819 | n1(nc(s2)CCc(ccc(c3OC)OC)c3)c2nnc1c(cc4)ccn4 |
| Targeted | | | |
| Diversity Library | | | |
| ChemDiv | ChemDiv | E218-0397 | C(C)(C1)(C(═O)NC2CCC(CC2)C)N(C3CCCCC3)C(c4n1c5c(c4) |
| Targeted | | | occ5)═O |
| Diversity Library | | | |
| ChemDiv | ChemDiv | E218-0232 | n1(CC2(C)C(═O)NC3CCCCC3)c(cc(oc(C)c4)c14)C(N2Cc5cccc |
| Targeted | | | (Br)c5)═O |
| Diversity Library | | | |
| ChemDiv | ChemDiv | E234-0018 | N1(c2ccc(cc20C)OC)C(═O)c3n(CC1(C)C(═O)NC4CCCCC4)c5c |
| Targeted | | | (cccc5)c3 |
| Diversity Library | | | |
| ChemDiv | ChemDiv | E234-0056 | C1(C)(Cn(c2c(c3)cccc2)c3C(═O)N1c4ccc(c5c4)OCCO5)C(═O) |
| Targeted | | | NC6CCCCC6 |
| Diversity Library | | | |
| ChemDiv | ChemDiv | E157-3455 | n(c(C)nn1)(n2)c1sc2NC(═O)c3cccc(C)c3 |
| Targeted | | | |
| Diversity Library | | | |
| ChemDiv | ChemDiv | E218-0201 | n1(CC2(C)C(═O)NC3CCCCC3)c(cc(oc(C)c4)c14)C(N2Cc(cc5) |
| Targeted | | | ccc5F)═O |
| Diversity Library | | | |
| ChemDiv | ChemDiv | E218-0425 | n(C1)(c2c(c3)occ2)c3C(N(CCN(C)C4CCCCC4)C1(C)C(═O)NC5CCC |
| Targeted | | | (CC5)C)═O |
| Diversity Library | | | |
| ChemDiv | ChemDiv | E234-0008 | N(Cc1ccccc1)(C2═O)C(C)(Cn(c3c(cccc3)c4)c24)C(═O)NC5CCCCC5 |
| Targeted | | | |
| Diversity Library | | | |
| ChemDiv | ChemDiv | E613-0091 | c1(NC(COCc(c2)noc2c(ccc(c34)OCO3)c4)═O)sc(c5c1C(N)═O)CCCC5 |
| Targeted | | | |
| Diversity Library | | | |
| ChemDiv | ChemDiv | E722-2603 | C1(NC(═O)C(SCc(c23)c4c(CCCC4)s2)═C3)═C(C)N(N(c5ccccc5) |
| Targeted | | | C1═O)C |
| Diversity Library | | | |
| ChemDiv | ChemDiv | E722-1395 | C1(NC(═O)C(SCc(c23)c(c(C)s2)C)═C3)═C(C)N(N(c4ccccc4)C1═O)C |
| Targeted | | | |
| Diversity Library | | | |
| ChemDiv | ChemDiv | E667-0223 | S(═O)(═O)(c(ccc1c2c3c[H]1)CCCC3)c2)Nc4c(cccn4)C |
| Targeted | | | |
| Diversity Library | | | |
| ChemDiv | ChemDiv | F083-0416 | N1(c2c(cc(OCO3)c3c2)C(N4)═O)C4═C(SC1═S)C(═O)NCC5CCCO5 |
| Targeted | | | |
| Diversity Library | | | |
| ChemDiv | ChemDiv | F083-0285 | N1(c2c(cc(Br)cc2)C(N3)═O)C3═C(SC1═S)C(NC)═O |
| Targeted | | | |
| Diversity Library | | | |
| ChemDiv | ChemDiv | F128-0042 | C(C(═O)N1CCCCC1)(SC(N2Cc3ccccc3)═S)═C2N |
| Targeted | | | |
| Diversity Library | | | |
| ChemDiv | ChemDiv | F128-0030 | N1(Cc2ccccc2)C(═S)SC(C(N)═O)═C1N |
| Targeted | | | |
| Diversity Library | | | |
| ChemDiv | ChemDiv | F201-0117 | N1(Cc(c(c2)C1═O)ccn2)c3c(F)ccc(F)c3 |
| Targeted | | | |
| Diversity Library | | | |
| ChemDiv | ChemDiv | F233-0420 | N1═C(C(═O)Nc2ccccc2)C(═CC(═O)N1c3ccc(cc3)F)OC |
| Targeted | | | |
| Diversity Library | | | |
| ChemDiv | ChemDiv | F233-0181 | N1(c2ccccc2C)C(═O)C═C(C(C(═O)Nc3ccccc3)═N1)OC |
| Targeted | | | |
| Diversity Library | | | |
| ChemDiv | ChemDiv | F260-0258 | c1(C(═O)Nc(ccc(c2s3)nc3NS(C)(═O)═O)c2)n[nH]c4c1CCc(c45) |
| Targeted | | | cc(cc5)OC |
| Diversity Library | | | |
| ChemDiv | ChemDiv | F255-0057 | c1(CCc(cc2)ccc2NC(═O)c3ccc(cc3)NC(C)═O)nc(c4n1c5ccccc5) |
| Targeted | | | cccc4 |
| Diversity Library | | | |
| ChemDiv | ChemDiv | F268-0090 | o1c(CCN(CC2)CCO2)nnc1SCC(═O)Nc3ccccc3 |
| Targeted | | | |
| Diversity Library | | | |
| ChemDiv | ChemDiv | F293-0001 | S(═O)(═O)(c1ccc(cc1)F)Nc(cnc(c2C(O)═O)N(CC)Cc3ccccc3)c2 |
| Targeted | | | |
| Diversity Library | | | |
| ChemDiv | ChemDiv | F294-0004 | S(c1ccccc1)(═O)(═O)Nc(ccc(c2C(O)═O)N(CC)Cc3ccccc3)c2 |
| Targeted | | | |
| Diversity Library | | | |
| ChemDiv | ChemDiv | F305-0036 | N(CCCC1C(═O)Nc2ccccc2OCC)(C1)c3ccc(nn3)c4ccccc4 |
| Targeted | | | |
| Diversity Library | | | |
| ChemDiv | ChemDiv | F293-0002 | S(═O)(═O)(Nc(cnc(c1C(O)═O)N(CC)Cc2ccccc2)c1)c3c(C)cc(cc3C)C |
| Targeted | | | |
| Diversity Library | | | |
| ChemDiv | ChemDiv | F293-0815 | S(═O)(═O)(c1ccc(c(F)c1)F)Nc(cnc(c2C(O)═O)N(C)C)c2 |
| Targeted | | | |
| Diversity Library | | | |
| ChemDiv | ChemDiv | F305-0129 | C(CCCN1c2ccc(nn2)c3ccccc3C)(C1)C(═O)N(CCCC)CC |
| Targeted | | | |
| Diversity Library | | | |
| ChemDiv | ChemDiv | F294-0006 | c1(cc(ccc1N(CC)Cc2ccccc2)NS(CC)(═O)═O)C(O)═O |
| Targeted | | | |
| Diversity Library | | | |
| ChemDiv | ChemDiv | F293-0526 | c1(cc(cnc1N(CCCC)CC)NS(CC)(═O)═O)C(O)═O |
| Targeted | | | |
| Diversity Library | | | |
| ChemDiv | ChemDiv | F293-0616 | S(═O)(═O)(Nc(cnc(c1C(O)═O)N(CC2)CCC2N3CCCCC3)c1)c4c(F) |
| Targeted | | | ccc(F)c4 |
| Diversity Library | | | |
| ChemDiv | ChemDiv | F305-0061 | C(CCCN1c2ccc(nn2)c3ccccc3)(C1)C(═O)N(CCC)CCC |
| Targeted | | | |
| Diversity Library | | | |
| ChemDiv | ChemDiv | F305-0007 | N(CCCC1C(═O)Nc2cccc(C)c2)(C1)c3ccc(nn3)c4ccccc4 |
| Targeted | | | |
| Diversity Library | | | |
| ChemDiv | ChemDiv | F293-0529 | c1(cc(cnc1N(CCCC)CC)NS(C)(═O)═O)C(O)═O |
| Targeted | | | |
| Diversity Library | | | |
| ChemDiv | ChemDiv | F294-0009 | c1(cc(ccc1N(CC)Cc2ccccc2)NS(C)(═O)═O)C(O)═O |
| Targeted | | | |
| Diversity Library | | | |
| ChemDiv | ChemDiv | F293-0762 | S(═O)(═O)(c1ccc(c2c1)CCCC2)Nc(cnc(c3C(O)═O)N(CC4)CCO4) |
| Targeted | | | c3 |
| Diversity Library | | | |
| ChemDiv | ChemDiv | F294-0010 | S(═O)(═O)(Nc(ccc(c1C(O)═O)N(CC)Cc2ccccc2)c1)c3cc(C)ccc3C |
| Targeted | | | |
| Diversity Library | | | |
| ChemDiv | ChemDiv | F281-0114 | c1(c(cccc1NC(COc(cc2)ccc2F)═O)ns3)n3 |
| Targeted | | | |
| Diversity Library | | | |
| ChemDiv | ChemDiv | F290-0671 | S(═O)(═O)(N(C)C)Nc(ccc(c1C(O)═O)N(CC2)CCN2CC(═O)N3CCCC3) |
| Targeted | | | c1 |
| Diversity Library | | | |
| ChemDiv | ChemDiv | F293-0436 | S(═O)(═O)(Nc(cnc(c1C(O)═O)N2CCCC2)c1)c3c(OC)ccc(OC)c3 |
| Targeted | | | |
| Diversity Library | | | |
| ChemDiv | ChemDiv | F293-0775 | S(═O)(═O)(Nc(cnc(c1C(O)═O)N(CC2)CCO2)c1)c3c(F)ccc(F)c3 |
| Targeted | | | |
| Diversity Library | | | |
| ChemDiv | ChemDiv | F294-0012 | S(═O)(═O)(Nc(ccc(c1C(O)═O)N(CC)Cc2ccccc2)c1)c3c(OC)ccc |
| Targeted | | | (OC)c3 |
| Diversity Library | | | |
| ChemDiv | ChemDiv | F294-0183 | S(═O)(═O)(N(C)C)Nc(ccc(c1C(O)═O)N(CC2)CCC2(C(N)═O) |
| Targeted | | | N3CCCCC3)c1 |
| Diversity Library | | | |
| ChemDiv | ChemDiv | F388-0026 | C1(═O)c2c(cccc2)N═CN1CCC(═O)Nc(cc3)ccc3OC |
| Targeted | | | |
| Diversity Library | | | |
| ChemDiv | ChemDiv | F407-0312 | c1(oc(c(CSc2nc(c(cccn3)c3)cc(O)n2)n1)C)c4ccc(cc4OC)OC |
| Targeted | | | |
| Diversity Library | | | |
| ChemDiv | ChemDiv | F500-0433 | c1(C(═O)Nc2cccc(Cl)c2)sc(nn1)COCC(═O)N(CC3)CCN3c4ccccc4 |
| Targeted | | | |
| Diversity Library | | | |
| ChemDiv | ChemDiv | F518-0014 | n1(ncn2)c2nc(C)cc1Sc3ccccc3NC(═O)c4cccc(C)c4 |
| Targeted | | | |
| Diversity Library | | | |
| ChemDiv | ChemDiv | F518-0049 | n1(ncn2)c2nc(C)cc1Sc3ccccc3NC(CC)═O |
| Targeted | | | |
| Diversity Library | | | |
| ChemDiv | ChemDiv | F542-0424 | N1(C(C)C)c(cc2)c(cc2c3noc(\C═C\c4ccccc4)n3)NC(═O)C1═O |
| Targeted | | | |
| Diversity Library | | | |
| ChemDiv | ChemDiv | F571-0001 | N12C(═CC(═O)N1)N═C(c3ccccc3)N═C2SCC(═O)Oc4ccccc4 |
| Targeted | | | |
| Diversity Library | | | |
| ChemDiv | ChemDiv | F571-0419 | N12C(═CC(═O)N1)N═C(c3ccc(cc3)OC)N═C2SCC(═O)Oc4ccccc4 |
| Targeted | | | |
| Diversity Library | | | |
| ChemDiv | ChemDiv | F617-0185 | n1(c(cc2)ccc2C(═O)NCc3cc(OC)ccc3OC)c4c(nn1)cccn4 |
| Targeted | | | |
| Diversity Library | | | |
| ChemDiv | ChemDiv | F685-1206 | c1(C(O)═O)cc(ccc1NC(C(C2)CC2)═O)N(C)CCCC |
| Targeted | | | |
| Diversity Library | | | |
| ChemDiv | ChemDiv | F687-1038 | c1(C(O)═O)cc(ccc1NC(C(C2)CC2)═O)N(CC3)CCN3c4ccc(cc4)Cl |
| Targeted | | | |
| Diversity Library | | | |
| ChemDiv | ChemDiv | F688-0002 | c12c(ccc(c1)C(N)═O)NC(═CC2═O)CSc3nnc[nH]3 |
| Targeted | | | |
| Diversity Library | | | |
| ChemDiv | ChemDiv | F680-0173 | N1(CC)c2c(cccc2)N═C(SCC(═O)NCc3ccccn3)C1═O |
| Targeted | | | |
| Diversity Library | | | |
| ChemDiv | ChemDiv | F684-0019 | S(═O)(═O)(c1ccc(cc1)F)Nc2ccc(cc2C(O)═O)N(CC3)CCN3c4ccc |
| Targeted | | | (cc4)OC |
| Diversity Library | | | |
| ChemDiv | ChemDiv | F688-0005 | [nH]1c(SCC(═CC2═O)Nc(c23)ccc(c3)C(N)═O)nnc1c4ccc(cc4)C |
| Targeted | | | |
| Diversity Library | | | |
| ChemDiv | ChemDiv | F726-1263 | n1c(c2ccccc2)nccc1N(CCCC3C(═O)NC4CC4)C3 |
| Targeted | | | |
| Diversity Library | | | |
| ChemDiv | ChemDiv | F781-0170 | n1(ncn2)c2nc(c(CC)c1Sc3ccccc3NC(═O)Nc4ccccc4)C |
| Targeted | | | |
| Diversity Library | | | |
| ChemDiv | ChemDiv | F792-1521 | c(C(═O)Nc(ccc(c12)OCO1)c2)(nnc3C4CCCN4C(C5CCCC5)═O)s3 |
| Targeted | | | |
| Diversity Library | | | |
| ChemDiv | ChemDiv | F781-0023 | n1(ncn2)c2nc(C)cc1Sc3ccccc3NC(═O)Nc4ccccc4 |
| Targeted | | | |
| Diversity Library | | | |
| ChemDiv | ChemDiv | F781-0201 | n1(ncn2)c2nc(c(CC)c1Sc3ccccc3NC(═O)Nc(ccc(c45)OCCO4)c5)C |
| Targeted | | | |
| Diversity Library | | | |
| ChemDiv | ChemDiv | F793-0015 | c(C(═O)Nc1cccc(F)c1)(nnc2C3CCCN3C(═O)Nc4ccc(cc4Cl)C)s2 |
| Targeted | | | |
| Diversity Library | | | |
| ChemDiv | ChemDiv | F781-0032 | n1(ncn2)c2nc(C)cc1Sc3ccccc3NC(═O)Nc4ccccc4Br |
| Targeted | | | |
| Diversity Library | | | |
| ChemDiv | ChemDiv | F781-0523 | n1(ncn2)c2nc(CC)cc1Sc3ccccc3NC(═O)Nc(cc4)ccc4Cl |
| Targeted | | | |
| Diversity Library | | | |
| ChemDiv | ChemDiv | F792-0003 | c(C(═O)Nc1cccc(F)c1)(nnc2C3CCCN3C(═O)c4ccc(cc4)Cl)s2 |
| Targeted | | | |
| Diversity Library | | | |
| ChemDiv | ChemDiv | F798-0626 | c12n(c(nn1)SCC(═O)NCc3ccco3)c(c4C(═O)N2Cc5ccco5)ccs4 |
| Targeted | | | |
| Diversity Library | | | |
| ChemDiv | ChemDiv | F818-0094 | S(═O)(═O)(c(ccc(c1S2)NC2═O)c1)N(C)C3CCCCC3 |
| Targeted | | | |
| Diversity Library | | | |
| ChemDiv | ChemDiv | F835-0135 | n12c(C(NN═C1SCc3ccccc3C)═O)cc(c4ccc(cc4)F)n2 |
| Targeted | | | |
| Diversity Library | | | |
| ChemDiv | ChemDiv | F912-0858 | c1(C(═O)Nc(cccc2C(NCCc(c3)c4c([nH]3)ccc(C)c4)═O)c2)sc(nn1) |
| Targeted | | | COC |
| Diversity Library | | | |
| ChemDiv | ChemDiv | F912-0859 | c1(C(═O)Nc(cccc2C(NCCc(c3)c4c([nH]3)ccc(Cl)c4)═O)c2)sc(nn1) |
| Targeted | | | COC |
| Diversity Library | | | |
| ChemDiv | ChemDiv | G199-0398 | N1(N═C(S2)CCC)C2═NC(CSC3═NC(c4ccc(cc4)C)═NC(═CC(═O) |
| Targeted | | | N5)N35)═CC1═O |
| Diversity Library | | | |
| ChemDiv | ChemDiv | G189-2182 | S(═O)(═O)(Nc1cc(on1)C)c2c(OC)ccc(c2)c(onc3C(OCC)═O)c3 |
| Targeted | | | |
| Diversity Library | | | |
| ChemDiv | ChemDiv | G199-0048 | N1(N═C(S2)CC)C2═NC(CSC3═NC(c4ccccc4)═NC(═CC(═O)N5) |
| Targeted | | | N35)═CC1═O |
| Diversity Library | | | |
| ChemDiv | ChemDiv | G199-2057 | N12C(═CC(═O)N1)N═C(c(ccc(c34)OCO3)c4)N═C2SCC(═O)N(C) |
| Targeted | | | Cc5ccccc5 |
| Diversity Library | | | |
| ChemDiv | ChemDiv | G747-0002 | C1(C(c2ccccc2)N(CC3)CCN3C)═C(O)C═C(N(Cc4ccccc4)C1═O)C |
| Targeted | | | |
| Diversity Library | | | |
| ChemDiv | ChemDiv | G784-0099 | c1(cc(s2)C(═O)Oc(ccc(c3ccn4)c4)c3)c2n(nc1c5ccccc5F)C |
| Targeted | | | |
| Diversity Library | | | |
| ChemDiv | ChemDiv | G786-1264 | c1(scc(c(cc2)ccn2)n1)NC(═O)CCS(c3ccccc3)(═O)═O |
| Targeted | | | |
| Diversity Library | | | |
| ChemDiv | ChemDiv | G821-0669 | c1(s2)c(C(NC═N1)═O)c(c2C(═O)N(CC3)CCC3C(═O)N(CC4) |
| Targeted | | | CC═C4c5ccccc5)C |
| Diversity Library | | | |
| ChemDiv | ChemDiv | G843-0432 | C(C═CC(N1Cc(cc2)ccc2Cl)═O)(═C1)C(═O)Nc3nnc(C)s3 |
| Targeted | | | |
| Diversity Library | | | |
| ChemDiv | ChemDiv | G856-6719 | c1(nnc2SCC(═O)Nc3sc(c4c3C(OCC)═O)CCCCC4)n2C(═CC(═O)N1)C |
| Targeted | | | |
| Diversity Library | | | |
| ChemDiv | ChemDiv | G857-0928 | N(C)(C(═O)c1c(N2C)ncc(CC)c1SC(C)CC)C2═O |
| Targeted | | | |
| Diversity Library | | | |
| ChemDiv | ChemDiv | G857-2309 | c12c(ncnc1NCCCOCC)n(nn2)CC |
| Targeted | | | |
| Diversity Library | | | |
| ChemDiv | ChemDiv | G889-0021 | c1(cc(ccc1N(CC)Cc2ccccc2)NC(═O)c3ccccc3C)C(O)═O |
| Targeted | | | |
| Diversity Library | | | |
| ChemDiv | ChemDiv | G890-1803 | c1(cc(cnc1N(CC2)CCN2CC(═O)N(CC)CC)NC(═O)c3ccc(cc3)Cl)C |
| Targeted | | | (O)═O |
| Diversity Library | | | |
| ChemDiv | ChemDiv | G890-0455 | c1(cc(cnc1N(CC2)CCN2c3c(C)ccc(Cl)c3)NC(CC)═O)C(O)═O |
| Targeted | | | |
| Diversity Library | | | |
| ChemDiv | ChemDiv | G889-0171 | c1(cc(ccc1N(CC2)CCC2(C(N)═O)N3CCCCC3)NC(CCCC)═O)C(O)═O |
| Targeted | | | |
| Diversity Library | | | |
| ChemDiv | ChemDiv | G890-0459 | c1(cc(cnc1N(CC2)CCN2c3c(C)ccc(Cl)c3)NC(CCCC)═O)C(O)═O |
| Targeted | | | |
| Diversity Library | | | |
| ChemDiv | ChemDiv | G890-0200 | c1(cc(cnc1N(CC2)CCN2c3ccc(cc3)Cl)NC(CC)═O)C(O)═O |
| Targeted | | | |
| Diversity Library | | | |
| ChemDiv | ChemDiv | J015-0261 | n1c(ccc(Br)c1C(NCCC2═CCCCC2)═O)n(cnn3)c3 |
| Targeted | | | |
| Diversity Library | | | |
| ChemDiv | ChemDiv | J015-0388 | n(c1)(cnn1)c2c(ccc(Cl)c2)OCC(═O)Nc3c(O)ccc(C(C)C)c3 |
| Targeted | | | |
| Diversity Library | | | |
| ChemDiv | ChemDiv | J021-3314 | N1(C)C(═O)c2c(cc(cc2)NC(CCc3onc(CSc4[nH]c(c5n4)ccc(Cl)c5) |
| Targeted | | | n3)═O)C1═O |
| Diversity Library | | | |
| ChemDiv | ChemDiv | J021-3320 | c1(NC(CCc2onc(CSc3[nH]c(c4n3)ccc(Cl)c4)n2)═O)nnc(CC)s1 |
| Targeted | | | |
| Diversity Library | | | |
| ChemDiv | ChemDiv | J035-0001 | c1(nc2)n(ncc1C(═O)NCc3cccc(Cl)c3)c(c24)CCCC4═O |
| Targeted | | | |
| Diversity Library | | | |
| ChemDiv | ChemDiv | J065-2258 | n1c(c2ccc(cc2)C)onc1CN(CCCC3C(═O)Nc4cc(C)ccc4O)C3 |
| Targeted | | | |
| Diversity Library | | | |
| ChemDiv | ChemDiv | K261-1972 | S1(═O)(═O)c2c(cccc2)N(C(SCc3ccccc3C)═N1)CCC |
| Targeted | | | |
| Diversity Library | | | |
| ChemDiv | ChemDiv | K784-4049 | c1(nc(C)c(c2O)Cl)n2ncc1C(═O)NCc(ccc(c34)OCO3)c4 |
| Targeted | | | |
| Diversity Library | | | |
| ChemDiv | ChemDiv | L062-0524 | c1(C(═O)Nc2ccccc2)sc(nn1)CNC(═O)Nc(cc3)ccc3C(OC)═O |
| Targeted | | | |
| Diversity Library | | | |
| ChemDiv | ChemDiv | G541-0792 | c1(CSc(c23)ccc(Cl)c2)c3n(nc1C(NCCC4═CCCCC4)═O)C |
| Targeted | | | |
| Diversity Library | | | |
| ChemDiv | ChemDiv | G550-0094 | S1(═O)(═O)c2c(cccc2)NC(C(═O)N(CC3)CCN3c4ccccc4)N1 |
| Targeted | | | |
| Diversity Library | | | |
| ChemDiv | ChemDiv | G695-0123 | S(═O)(═O)(N(CC1)CCN1c2ncccn2)c3n[nH]c(C(OCC)═O)c3 |
| Targeted | | | |
| Diversity Library | | | |
| ChemDiv | ChemDiv | L378-0331 | c1(N(CC2)CCN2C(═O)Nc3ccc(cc3Cl)C)nnnn1c4cccc(CC)c4 |
| Targeted | | | |
| Diversity Library | | | |
| ChemDiv | ChemDiv | L662-0326 | n1c(Oc(cc2)ccc2C(═O)Nc(ccc(c34)OCCO3)c4)ccnc1c5cccc(C)c5 |
| Targeted | | | |
| Diversity Library | | | |
| ChemDiv | ChemDiv | M040-0342 | n1c(Cc2scc(c3ccc(cc3)Cl)n2)onc1c(ccc(c45)OCO4)c5 |
| Targeted | | | |
| Diversity Library | | | |
| ChemDiv | ChemDiv | M348-1088 | c(c(C)nn1c2ccc(nn2)c3ccc(c(C)c3)C)(S(NCCN4CCCCC4C)(═O)═O) |
| Targeted | | | c1C |
| Diversity Library | | | |
| Enamine 2 | Enamine | T5369197 | SC1═NC(C)C(═O)N1Cc1ccccc1 |
| Enamine 2 | Enamine | T5274408 | O═C(NCc1ccco1)COC(═O)c1ccc(cc1)S(═O)(═O)NCc1ccco1 |
| Enamine 2 | Enamine | T5256544 | Fc1ccc(\C═C/C(═O)Oc2cc(C)oc(═O)c2)cc1 |
| Enamine 2 | Enamine | T5220323 | CCCn1c(═O)c2ccccc2n2c(S)nnc12 |
| Enamine 2 | Enamine | T5311662 | CC1OC(C)CN(C1)C(═O)C(C)OC(═O)c1ccc2ncsc2c1 |
| Enamine 2 | Enamine | T5233302 | CN(C)Cc1n[nH]c(═S)o1 |
| Enamine 2 | Enamine | T0513-3552 | O═C(CSCc1ccco1)c1ccco1 |
| Enamine 2 | Enamine | T0510-7133 | OC(═O)CCCCN1C(═S)S/C(═C\c2ccc(C)o2)/C1═O |
| Enamine 2 | Enamine | T0509-6651 | O═c1n(c2ccccc2)c2nnc(S)n2c2ccccc12 |
| Enamine 2 | Enamine | T0511-0483 | CC(═O)Oc1cn(C(C)C)c2nc(C)n3c(nc4ccccc4c3═O)c12 |
| Enamine 2 | Enamine | T0508-0820 | CCN1C(═O)\C(═N\N2C(═S)SCC2═O)/c2ccccc12 |
| Enamine 2 | Enamine | T5407468 | COC(═O)CC1C(═O)NCCN1c1ncnc2sccc12 |
| Enamine 2 | Enamine | T5333269 | O═C(Nc1ccc(cc1)N1CCOCC1)CN(C)Cn1nc(Nc2ccccc2F)sc1═S |
| Enamine 2 | Enamine | T5327980 | O═C(CN1CCC(═CC1)c1ccccc1)Nc1ccc(cc1)NC(═O)C |
| Enamine 2 | Enamine | T5232715 | CCC(═O)N(C1CC1)c1nnc(S)s1 |
| Enamine 2 | Enamine | T5233113 | CC(═O)N1CCN(CC1)C(═O)c1sccc1c1ccccc1 |
| Enamine 2 | Enamine | T5477163 | Cn1nc(Nc2ccccc2N)cc1c1ccccc1 |
| Enamine 2 | Enamine | T5468381 | Clc1cc2CN(COc2c2ncccc12)CC1CCCO1 |
| Enamine 2 | Enamine | T5394435 | COc1ccc(cn1)NC(═O)COc1ccc(C)c(C)c1 |
| Enamine 2 | Enamine | T0519-5027 | CCN1CCN(CC1)Cn1nc(c2c[nH]c3ccccc23)n(CC2CCCO2)c1═S |
| Enamine 2 | Enamine | T5237912 | S═C(NC1CC2CCC1C2)Nn1c(═O)[nH]c2ccccc2c1═O |
| Enamine 2 | Enamine | T5236464 | CCOC(═O)c1sc(N)c(C#N)c1CSc1nnc(C2CC2)n1N |
| Enamine 2 | Enamine | T0518-9672 | C═CC/N═c1/scc(c2ccco2)n/1/N═C(\C)/CC |
| Enamine 2 | Enamine | T0519-5849 | O═C(Nc1ccc(cc1)S(═O)(═O)N)C(C)NCC1COc2ccccc2O1 |
| Enamine 2 | Enamine | T5450906 | O═C(CSCC(═O)O)Nc1scc(n1)c1cccs1 |
| Enamine 2 | Enamine | T0520-3534 | N#C\C(═C(\C)/N)\C(═O)CSc1nc(cn1N)c1ccccc1 |
| Enamine 2 | Enamine | T0504-7478 | S═C1S/C(═C\c2cccc(OCc3c(C)no[n+]3[O—])c2)/C(═O)N1 |
| Enamine 2 | Enamine | T0504-5521 | CC(C)NP(═O)(c1ccccc1)C(C)(C)C |
| Enamine 2 | Enamine | T0504-7486 | Oc1ccc(C(═O)Cc2nc3ccccc3s2)c(O)c1 |
| Enamine 2 | Enamine | T0504-9168 | CCOC(═O)c1sc(═S)n(CC═C)c1NC(═O)CC |
| Enamine 2 | Enamine | T0504-8837 | CC1═C(C)CSC2(C1)C(═NN(c1ccccc1)C2═O)C |
| Enamine 2 | Enamine | T0520-0527 | Sc1scc(n1)c1ccccc1 |
| Enamine 2 | Enamine | T0504-6549 | CNC(═S)Nc1ccc2[nH]c(═O)[nH]c2c1 |
| Enamine 2 | Enamine | T0504-7177 | Nc1nc(═O)c2cccnc2s1 |
| Enamine 2 | Enamine | T5405984 | O═C(COc1ccc(Cl)cc1)NN1CC(═O)NC1═O |
| Enamine 2 | Enamine | T5381504 | Oc1ccc(cc1)N1CCN(CC1)c1nc(nc2ccccc12)c1cccnc1 |
| Enamine 2 | Enamine | T0517-4492 | COC(═O)CC1C(═O)NCCN1C(═O)CSc1nc2ccccc2s1 |
| Enamine 2 | Enamine | T0518-7941 | OC(═O)CCc1nc2ccccc2c(═O)n1Cc1ccc2OCOc2c1 |
| Enamine 2 | Enamine | T0503-7718 | N#CCCn1nc(C)c2c1N═C(OP2(═S)N(CC)CC)c1ccc(I)cc1 |
| Enamine 2 | Enamine | T5344405 | O═C1OCCC1Sc1nnc(Cc2cccc3ccccc23)n1N |
| Enamine 2 | Enamine | T5345416 | COCCn1c(SCC(═O)NC(═O)CN2CCCC2═O)nc2ccccc2c1═O |
| Enamine 2 | Enamine | T5441865 | COc1cc(/C═N\NC(═O)Cn2ncn(c3ccccc3)c2═O)ccc1OC(═O) |
| | | | CC1SC(═O)NC1═O |
| Enamine 2 | Enamine | T5441567 | O═c1sc2c([nH]1)SC1═C(C2c2ccccc2O)C(═O)c2ccccc2C1═O |
| Enamine 2 | Enamine | T0518-9283 | O═C(N/N═C1\c2cccc3cccc(C/1═O)c23)C1COc2ccccc2O1 |
| Enamine 2 | Enamine | T5246216 | CCC(N(C)C)c1n[nH]c(═S)o1 |
| Enamine 2 | Enamine | T0516-1649 | CC(CC)NC(═S)N |
| Enamine 2 | Enamine | T0507-4301 | CCN1CCN(CC1)Cc1c(O)ccc2ccccc12 |
| Enamine 2 | Enamine | T0513-8817 | N#Cc1c(NC(═O)/C═C\c2ccco2)sc2CCCCc12 |
| Enamine 2 | Enamine | T0502-0855 | O═C1C2(C)CN3CC1(C)C(═O)C(C)(C3)C2═O |
| Enamine 2 | Enamine | T0504-1589 | Cc1cc(C)nc(NNc2ccccc2)n1 |
| Enamine 2 | Enamine | T0504-1872 | CCOC(═O)C1═C(C)NC(═O)NC1c1cn(nc1C)c1ccccc1 |
| Enamine 2 | Enamine | T0513-0928 | CCS(═O)(═O)c1ccc2oc(SCC(═O)Nc3ccc4OCCOc4c3)nc2c1 |
| Enamine 2 | Enamine | T5213648 | COCCN(C(═O)C)c1c(N)n(CCC)c(═O)n(CC(═O)Nc2cc(Cl)cc(Cl)c2) |
| | | | c1═O |
| Enamine 2 | Enamine | T0513-7553 | NC(═O)c1cc(sc1N)c1ccccc1 |
| Enamine 2 | Enamine | T0519-3366 | Brc1ccc2nc(COC(═O)c3cc(nc4ccccc34)c3ccco3)cc(═O)n2c1 |
| Enamine 2 | Enamine | T5211011 | Cc1cccc(c1)n1[nH]c(═S)sc1═S |
| Enamine 2 | Enamine | T5439822 | O═c1[nH]c2ccc(cc2o1)C(═O)CSc1nnc(c2ccco2)n1C1CCCCC1 |
| Enamine 2 | Enamine | T5414780 | COc1cccc(c1)c1nnc(SCN2C(═O)c3ccccc3C2═O)n1Cc1ccco1 |
| Enamine 2 | Enamine | T5427681 | FC(F)Oc1ccc(cc1)\C═C1\SC(═O)N(CCNC(═O)C2COc3ccccc3O2) |
| | | | C/1═O |
| Enamine 2 | Enamine | T5241112 | Oc1ccccc1C(═O)c1cnn(C(═S)NC2CCCCC2)c1N |
| Enamine 2 | Enamine | T5245818 | Oc1ccc(cc1)N1CCN(CC1)C(═O)c1cccc(c1)S(═O)(═O)N1CCc2ccccc2C1 |
| Enamine 2 | Enamine | T5517889 | O═C(NC(C(C)C)c1nc2ccccc2[nH]1)C1COc2ccccc2O1 |
| Enamine 2 | Enamine | T0518-4938 | CCc1ccc(cc1)Nc1nnc(SCC(═O)O)s1 |
| Enamine 2 | Enamine | T0518-5871 | CCOc1cc(ccc1OCC)C(═O)c1ccccc1C(═O)O |
| Enamine 2 | Enamine | T0518-4530 | c1ccc2OCC(CNc3ncnc4c3oc3ccccc43)Oc2c1 |
| Enamine 2 | Enamine | T0518-5876 | NNc1ccc(cn1)S(═O)(═O)N1CC(C)CC(C)C1 |
| Enamine 2 | Enamine | T5217358 | CN1CCC(CC1)N(C)Cn1ncn(c2ccccc2)c1═S |
| Enamine 2 | Enamine | T0505-8800 | CSC(═S)N/N═C(\C)/c1ccccc1 |
| Enamine 2 | Enamine | T0505-9441 | O═C(/C═C\c1ccco1)N(C)c1ccccc1C(═O)O |
| Enamine 2 | Enamine | T0518-6140 | S═c1n(CN2CCC(═CC2)c2ccccc2)nc2sc3ccccc3n12 |
| Enamine 2 | Enamine | T5218273 | O═C(COC(═O)C1═NN(C(═O)CC1)c1ccccc1)NC1CCCC1 |
| Enamine 2 | Enamine | T0515-7499 | N#C/C(═C\C1═CCC2CC1C2(C)C)/C(═O)N |
| Enamine 2 | Enamine | T0516-6822 | OC(═O)CC(Sc1ncnc2sc3CCCCc3c12)C(═O)O |
| Enamine 2 | Enamine | T0514-1693 | O═C(Nc1nnc(s1)C1CC1)c1ccc2ncsc2c1 |
| Enamine 2 | Enamine | T0514-2924 | CN1CCN(CC1)NC(═S)N═P(N1CCOCC1)(c1ccccc1)C(C)(C)C |
| Enamine 2 | Enamine | T0517-3239 | CC(═O)NCCCc1[nH]n(c2ccccc2)c(═O)c1NC(═O)c1ccccc1 |
| Enamine 2 | Enamine | T0515-5872 | COc1ccc(cc1)C(═O)NC(C(C)C)C(═O)N1CCN(CC1)c1ccccc1O |
| Enamine 2 | Enamine | T0516-9705 | CCCSc1[nH]c2ccc(cc2n1)S(═O)(═O)N1CCOCC1 |
| Enamine 2 | Enamine | T0512-6296 | NC(═O)CSc1oc2ccc(cc2n1)S(═O)(═O)N1CCOCC1 |
| Enamine 2 | Enamine | T0507-5894 | COc1cccc(c1)N(CCN1C(═O)c2ccccc2C1═O)C(═O)C(C)C |
| Enamine 2 | Enamine | T0517-1776 | O═C(COC(═O)c1nn(C)c(═O)c2ccccc12)NC1CC1 |
| Enamine 2 | Enamine | T0507-5444 | O═C(\C═C/c1ccccc1)Nc1ccc(O)cc1 |
| Enamine 2 | Enamine | T5214924 | O═C(OCc1nc2sc3CCCCc3c2c(═O)[nH]1)c1cc2CCCc2s1 |
| Enamine 2 | Enamine | T0517-0533 | Nc1cccc2c1C(═O)N(C(═O)c1cccc(c1)S(═O)(═O)N1CCOCC1)C2═O |
| Enamine 2 | Enamine | T0506-8624 | Cc1ccc(C)c(c1)C(═O)OCC(═O)C(C)(C)C |
| Enamine 2 | Enamine | T5225742 | COc1cc(CC(═O)OCC(═O)N(C)C2CCCCC2)cc(OC)c1OC |
| Enamine 2 | Enamine | T0504-5175 | CCOC(═O)c1oc2nc(cc(c2c1)C(F)(F)F)c1cccs1 |
| Enamine 2 | Enamine | T5221648 | Cc1occc1C(═O)CC#N |
| Enamine 2 | Enamine | T5223687 | O═C(CN1CCOCC1)N(C)C1(CCCCC1═O)c1ccccc1Cl |
| Enamine 2 | Enamine | T5337394 | c1ccc(cn1)c1nnc(c2ccccc2)c(n1)c1ccccc1 |
| Enamine 2 | Enamine | T5338184 | NC(═O)c1nn(C(C)C)c(═O)c2ccccc12 |
| Enamine 2 | Enamine | T5442194 | O═C(Nc1ccccc1N1CCOCC1)c1cc(nn1c1ccccc1)C1CC1 |
| Enamine 2 | Enamine | T5337098 | CC(C)Cn1c(SCn2nnc3ccccc3c2═O)nc2ccccc2c1═O |
| Enamine 2 | Enamine | T5337414 | CC(C)Cn1nc(C)c(\C═C2\N(C)C(═S)N(C)C/2═O)c1Cl |
| Enamine 2 | Enamine | T5384395 | N#Cc1c(S)[nH]c(═O)cc1C(F)(F)F |
| Enamine 2 | Enamine | T0515-5826 | O═C(CCc1nc2ccccc2s1)OC1CCCCC1═O |
| Enamine 2 | Enamine | T0513-8359 | NC(═O)c1c(N)sc2CCCCCc12 |
| Enamine 2 | Enamine | T0501-9604 | CN(C)/C═N/C1═NN(CC1)c1ccccc1 |
| Enamine 2 | Enamine | T0502-3140 | S═c1[nH]ncc2ccccc12 |
| Enamine 2 | Enamine | T0501-4765 | OCCN(CCO)C1═CC(═O)C(═CC1═O)C |
| Enamine 2 | Enamine | T0500-0203 | O═C(NNc1ccccc1)C(═C)C |
| Enamine 2 | Enamine | T5539359 | N#Cc1ccsc1NC(═O)CSc1nnc(NC2CC2)s1 |
| Enamine 2 | Enamine | T5526249 | Clc1c2ccccc2sc1c1nnc(S)n1CCN1CCOCC1 |
| Enamine 2 | Enamine | T5232474 | CCN1C(═O)C(═CNc2cc(ccc2O)S(═O)(═O)CC)C(═O)N(CC)C1═S |
| Enamine 2 | Enamine | T5527035 | Sc1nnc(Cc2cccc3ccccc23)n1\N═C\c1ccco1 |
| Enamine 2 | Enamine | T5393666 | CCn1c2ccc(cc2[nH]c(═O)c1═O)C(═O)N1CCN(CC1)c1ccccc1O |
| Enamine 2 | Enamine | T0515-3106 | O═c1[nH]c2cccc3cccc1c23 |
| Enamine 2 | Enamine | T5426338 | CCCNC(═O)c1ccc(cc1)C1SCCCS1 |
| Enamine 2 | Enamine | T5426076 | O═C1c2ccccc2C(═O)N1CSc1nnc(C)c(═O)n1N |
| Enamine 2 | Enamine | T5499746 | NC(═S)N1CCOCC1 |
| Enamine 2 | Enamine | T5499806 | OC(═O)CC1Sc2nnc(c3cccc(c3)S(═O)(═O)N(C)C)n2N═C1c1ccccc1 |
| Enamine 2 | Enamine | T5535917 | Nc1nc(N)nc(n1)CN1C(═O)NC(c2ccccc2)(c2ccccc2)C1═O |
| Enamine 2 | Enamine | T5535808 | Cc1scc(CSc2nnc(Cc3cccc4ccccc34)n2N)n1 |
| Enamine 2 | Enamine | T5535170 | Oc1ccc(cc1)N1CCN(CC1)C(═O)CSc1nccn1c1cccc(F)c1 |
| Enamine 2 | Enamine | T5535492 | COc1ccc(CCC(═O)NNc2ccccc2)cc1 |
| Enamine 2 | Enamine | T5508565 | Cc1n[nH]/c(═N\C(═O)c2cn(nc2c2cccnc2)c2ccccc2)/o1 |
| Enamine 2 | Enamine | T5221304 | COC(═O)C1Cc2ccccc2CN1Cn1nc2sc3ccccc3n2c1═S |
| Enamine 2 | Enamine | T5504267 | CC(C)Nc1nn(CN2CCN(CC2)C(═O)c2ccccc2)c(═S)s1 |
| Enamine 2 | Enamine | T5303011 | COc1ccc(cc1)Nc1nnc(s1)SC1CCOC1═O |
| Enamine 2 | Enamine | T5512014 | N#Cc1ccc(cc1)NC(═O)C(C)N1CCN(CC1)c1ccc(O)cc1 |
| Enamine 2 | Enamine | T5495590 | CC(═O)NCc1ccc(o1)C(═O)CSc1nnc2sc3ccccc3n12 |
| Enamine 2 | Enamine | T5495341 | CCOC(═O)C1═C(CSc2nc3scc(c4cccs4)c3c(═O)[nH]2)NC(═O)NC1C |
| Enamine 2 | Enamine | T5495808 | Oc1ccc(cc1)N1CCN(CC1)CC(═O)c1[nH]ccc1 |
| Enamine 2 | Enamine | T5539084 | Cc1cccc(OCCNC(═O)c2sc3nc[nH]c(═O)c3c2C)c1 |
| Enamine 2 | Enamine | T5342884 | OC(═O)\C═C/c1cc(Cl)c2OCOc2c1 |
| Enamine 2 | Enamine | T5475332 | COc1cc(NS(═O)(═O)c2cccs2)c(C)cc1OC |
| Enamine 2 | Enamine | T5350666 | O═C1N(c2ccccc2)C(═S)N2CCCC12 |
| Enamine 2 | Enamine | T0514-1466 | CC(═C)CNC(═S)NN |
| Enamine 2 | Enamine | T0514-3281 | NNc1ccc(cc1)S(═O)(═O)c1ccc(cc1)C(C)C |
| Enamine 2 | Enamine | T0515-0782 | CCCCOC(═O)C1COc2ccccc2O1 |
| Enamine 2 | Enamine | T0518-9662 | S═C(NCCc1ccccc1)NNC1═NCCCCC1 |
| Enamine 2 | Enamine | T0400-1492 | S═C(NC(═O)c1ccccc1)Nc1ccccc1O |
| Enamine 2 | Enamine | T5473175 | Oc1c(ccc2cccnc12)C(N1CCN(CC1)c1ccccn1)c1ccccn1 |
| Enamine 2 | Enamine | T0518-0732 | Sc1nnc([nH]1)C(C)C |
| Enamine 2 | Enamine | T5372994 | SCCn1c(═S)[nH]c2ccccc2c1═O |
| Enamine 2 | Enamine | T0518-7999 | NC(═S)NCCc1ccccc1 |
| Enamine 2 | Enamine | T0510-3348 | NNC(═S)NC1CCCCC1C |
| Enamine 2 | Enamine | T0518-0326 | CCOc1cc(N2CCOCC2)c(OCC)cc1NC(═O)C1═NN(C(═O)CC1) |
| | | | c1ccccc1 |
| Enamine 2 | Enamine | T0506-4134 | Sc1nc2nc(cc(c2c(O)n1)C(F)(F)F)c1cccs1 |
| Enamine 2 | Enamine | T0508-1660 | CCOc1cccc(/C═N\n2c(S)nnc2c2ccc(Cl)cc2)c1O |
| Enamine 2 | Enamine | T0510-3343 | SC1═NCC(C)(C)CN1 |
| Enamine 2 | Enamine | T5355464 | O═C(NCc1ccc2OCOc2c1)C(C)NC1CCCc2ccccc12 |
| Enamine 2 | Enamine | T5364849 | Oc1ccccc1C(═O)c1cc(N2CCOCC2)c2nc3ccccc3c(═O)n2c1 |
| Enamine 2 | Enamine | T0514-0186 | NCCN1C(═O)S/C(═C\c2ccc(Cl)c(Cl)c2)/C1═O |
| Enamine 2 | Enamine | T0512-4738 | CC(═O)Oc1ccc(cc1)OC(═O)C |
| Enamine 2 | Enamine | T0513-3035 | CCOc1ccccc1NC(═S)NNc1nc2ccccc2o1 |
| Enamine 2 | Enamine | T0519-7535 | O═C(NCCCN1CCOCC1)c1cc(nc2ccccc12)c1ccco1 |
| Enamine 2 | Enamine | T0507-5550 | Sc1nnc(c2cccc(c2)S(═O)(═O)N2CCCC2)n1c1ccccc1 |
| Enamine 2 | Enamine | T0515-8376 | O═C(Oc1ccc2OCOc2c1)C(C)N1C(═O)c2ccccc2C1═O |
| Enamine 2 | Enamine | T5438075 | Cc1ccnc(SCC(═O)N)c1C#N |
| Enamine 2 | Enamine | T0515-3065 | Oc1ccc(cc1)N1CCNCC1 |
| Enamine 2 | Enamine | T0515-7072 | NNC(═O)CC1═NNC(═O)C1 |
| Enamine 2 | Enamine | T0504-0502 | N#CCCn1nc(/C═N\c2ccc(O)cc2)c(C)c1 |
| Enamine 2 | Enamine | T0516-9998 | CCN(CC)S(═O)(═O)c1cccc(c1)c1nnc(S)[nH]1 |

The eGFP secondary assay evaluated the hits identified from the primary screen. The compounds were judged off the % eGFP disruption to determine the Cas9 inhibition and the assay Zscore. Similar to the primary assay, compounds that had a Zscore>3 were selected as hits and are detailed in Tables 5A and 5B. The hits from the secondary screens will be processed through tertiary screens of dose studies and HiBit Assay as detailed herein.

TABLE 5A
eGFP Hits for SaCas9 Inhibitors

| Above 3 | Library | Vendor | |
|---|---|---|---|
| sigma | Name | ID | Smile |
| Yes | ChemDiv Targeted Diversity Library | D513- 3628 | 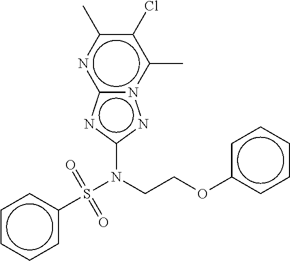 |
| | | | c(N(CCOc1ccccc1)S(c2ccccc2)(═O)═O)(nn3c4nc(c(Cl)c3C)C)n4 |
| Yes | ChemDiv Targeted Diversity Library | D278- 0547 | 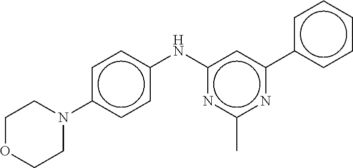 |
| | | | c1(Nc(cc2)ccc2N(CC3)CCO3)cc(c4ccccc4)nc(C)n1 |
| Yes | ChemDiv Targeted Diversity Library | D421- 0876 | 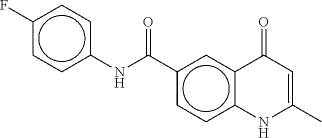 |
| | | | c12c(ccc(c1)C(═O)Nc(cc3)ccc3F)NC(═CC2═O)C |
| Yes | ChemDiv Targeted Diversity Library | D297- 0031 | 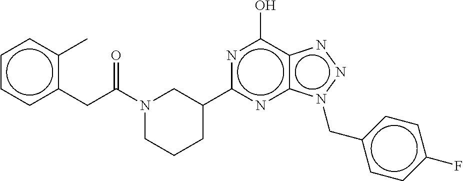 |
| | | | c12c(c(nc(C(CCCN3C(═O)Cc4ccccc4C)C3)n1)O)nnn2Cc(cc5)ccc5F |
| Yes | ChemDiv Targeted Diversity Library | C243- 0026 | 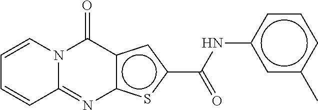 |
| | | | c1(C2═O)c(sc(C(═O)Nc3cccc(C)c3)c1)N═C(C═CC═C4)N24 |
| Almost | ChemDiv Targeted Diversity Library | C066- 3867 | 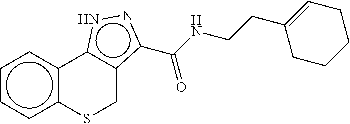 |
| | | | c1(CSc(c23)cccc2)c3[nH]nc1C(NCCC4═CCCCC4)═O |
| Almost | Enamine 1 | T0502- 0200 | 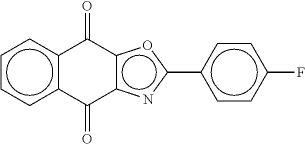 |
| | | | Fc1ccc(cc1)c1nc2C(═O)c3ccccc3C(═O)c2o1 |
| Yes | ChemDiv Targeted Diversity Library | D664- 0047 | 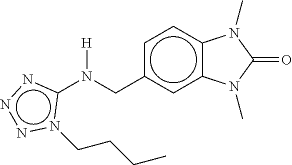 |
| | | | C1(═O)N(C)c2c(cc(cc2)CN([H])c3nnnn3CCCC)N1C |
| Yes | ChemDiv Targeted Diversity Library | D727- 0717 | 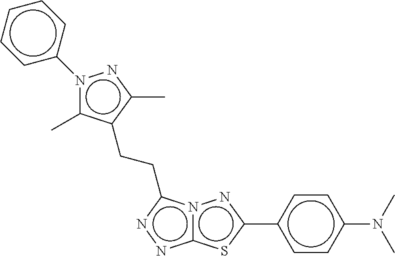 |
| | | | n1(c(CCc(c(C)nn2c3ccccc3)c2C)nn4)c4sc(c(cc5)ccc5N(C)C)n1 |
| Yes | ChemDiv Targeted Diversity Library | D686- 0195 | 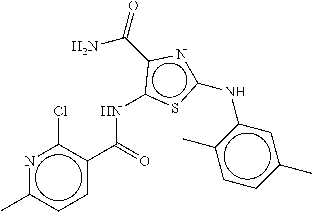 |
| | | | c1(NC(═O)c2ccc(nc2Cl)C)sc(nc1C(N)═O)Nc3cc(C)ccc3C |
| Yes | ChemDiv Targeted Diversity Library | D727- 0768 | 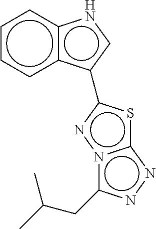 |
| | | | n1(n2)c(nnc1CC(C)C)sc2c3c4c([nH]c3)cccc4 |
| Almost | ChemDiv Targeted Diversity Library | D727- 0740 | 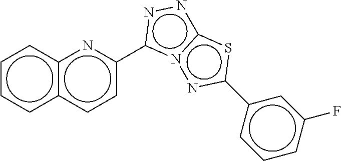 |
| | | | n12c(nnc1c3ccc(c4n3)cccc4)sc(c5cccc(F)c5)n2 |
| Almost | ChemDiv Targeted Diversity Library | D715- 2438 | 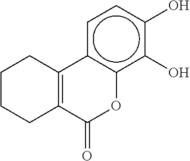 |
| | | | c12c(ccc(O)c1O)C3═C(C(═O)O2)CCCC3 |
| Almost | ChemDiv Targeted Diversity Library | D727- 0417 | 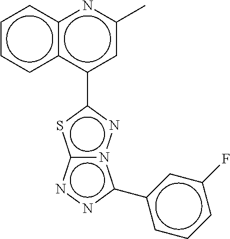 |
| | | | n12c(nnc1c3cccc(F)c3)sc(c(cc4C)c5c(n4)cccc5)n2 |
| Almost | ChemDiv Targeted Diversity Library | D727- 0059 | 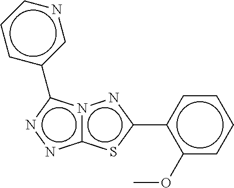 |
| | | | n12c(nnc1c(cccn3)c3)sc(c4ccccc4OC)n2 |
| Yes | ChemDiv Targeted Diversity Library | E922- 0258 | 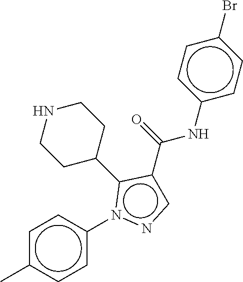 |
| | | | c(cnn1c2ccc(cc2)C)(C(═O)Nc(cc3)ccc3Br)c1C(CC4)CCN4 |
| Yes | ChemDiv Targeted Diversity Library | F255- 0057 | 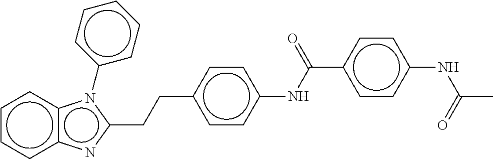 |
| | | | c1(CCc(cc2)ccc2NC(═O)c3ccc(cc3)NC(C)═O)nc(c4n1c5ccccc5)cccc4 |
| Yes | ChemDiv Targeted Diversity Library | E234- 0006 | 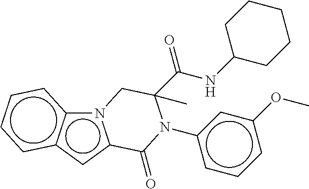 |
| | | | C1(C)(Cn(c2c(c3)cccc2)c3C(═O)N1c4cccc(OC)c4)C(═O)NC5CCCCC5 |
| Yes | ChemDiv Targeted Diversity Library | F083- 0285 | 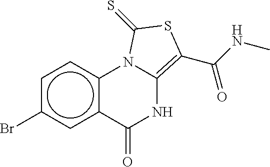 |
| | | | N1(c2c(cc(Br)cc2)C(N3)═O)C3═C(SC1═S)C(NC)═O |
| Yes | ChemDiv Targeted Diversity Library | E234- 0008 | 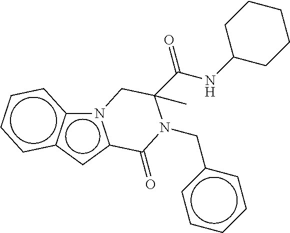 |
| | | | N(Cc1ccccc1)(C2═O)C(C)(Cn(c3c(cccc3)c4)c24)C(═O)NC5CCCCC5 |
| Yes | ChemDiv Targeted Diversity Library | F305- 0061 | 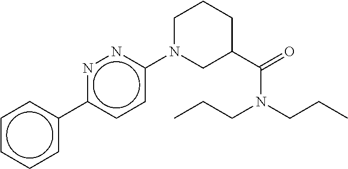 |
| | | | C(CCCN1c2ccc(nn2)c3ccccc3)(C1)C(═O)N(CCC)CCC |
| Yes | ChemDiv Targeted Diversity Library | F305- 0036 | 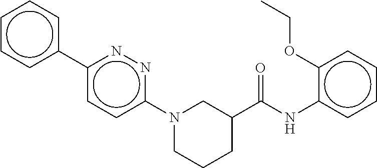 |
| | | | N(CCCC1C(═O)Nc2ccccc2OCC)(C1)c3ccc(nn3)c4ccccc4 |
| Almost | ChemDiv Targeted Diversity Library | E234- 0018 | 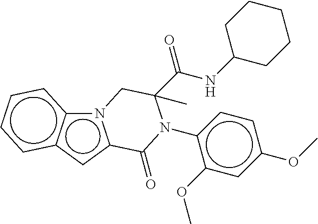 |
| | | | N1(c2ccc(cc2OC)OC)C(═O)c3n(CC1(C)C(═O)NC4CCCCC4)c5c(cccc5)c3 |
| Almost | ChemDiv Targeted Diversity Library | D226- 0165 | 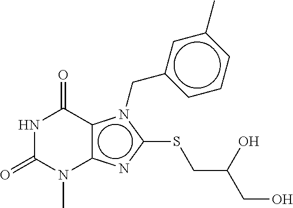 |
| | | | c12c(C(NC(═O)N1C)═O)n(c(SCC(CO)O)n2)Cc3cccc(C)c3 |
| Yes | ChemDiv Targeted Diversity Library | E722- 2652 | 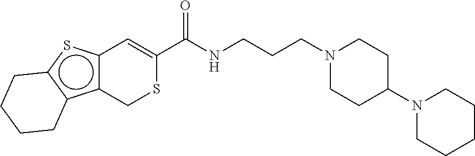 |
| | | | c12c(CSC(C(NCCCN(CC3)CCC3N4CCCCC4)═O)═C1)c5c(CCCC5)s2 |
| Yes | ChemDiv Targeted Diversity Library | F128- 0030 | 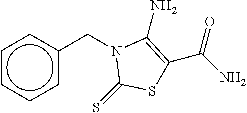 |
| | | | N1(Cc2ccccc2)C(═S)SC(C(N)═O)═C1N |

TABLE 5B
Hits for Secondary Assay of SaCas9

| | | | | | | | Normalized | | | | | | |
|---|---|---|---|---|---|---|---|---|---|---|---|---|---|
| | | | | | | | eGFP | | Normalized | | | | |
| | Cherry | | | | | | Disruption | | Inhibition | | Z score | | |
| eGFP | Pick | SDA | SDA | | | | Rep | Rep | Rep | Rep | Rep | Rep | Avg |
| Well | Well | Plate | Well | Library | Vendor ID | Smile | 1 | 2 | 1 | 2 | 1 | 2 | Z |
| A5 | C03 | 1395 | D01 | Enamine | T0502-0200 | Fc1ccc(cc1)c1nc2C(═O) | 64.909 | 64.481 | 35.091 | 35.519 | 3.528 | 2.749 | 3.138 |
| | | | | 1 | | c3ccccc3C(═O)c201 | | | | | | | |
| A11 | C09 | 3433 | A16 | Chem Div7 | C066-3867 | c1(CSc(c23)cccc2)c3[nH] | 60.813 | 66.352 | 39.187 | 33.648 | 3.940 | 2.604 | 3.272 |
| | | | | | | nc1C(NCCC4═CCCCC4)═O | | | | | | | |
| D7 | I05 | 3438 | G04 | ChemDiv | C243-0026 | c1(C2═O)c(sc(C(═O) | 69.530 | 59.481 | 30.470 | 40.519 | 3.063 | 3.136 | 3.100 |
| | | | | 7 | | Nc3cccc(C)c3)c1)N═C | | | | | | | |
| | | | | | | (C═CC═C4)N24 | | | | | | | |
| E17 | L07 | 3451 | P15 | Chem Div | D278-0547 | c1(Nc(cc2)ccc2N(CC3) | 44.472 | 56.693 | 55.528 | 43.307 | 5.583 | 3.352 | 4.467 |
| | | | | 7 | | CCO3)cc(c4ccccc4)nc(C)n1 | | | | | | | |
| F5 | M03 | 3452 | G08 | ChemDiv | D297-0031 | c12c(c(nc(C(CCCN3C(═O) | 64.041 | 56.575 | 35.959 | 43.425 | 3.615 | 3.361 | 3.488 |
| | | | | 7 | | Cc4ccccc4C)C3)n1)O) | | | | | | | |
| | | | | | | nnn2Cc(cc5)ccc5F | | | | | | | |
| F13 | N03 | 3459 | O02 | ChemDiv | D421-0876 | c12c(ccc(c1)C(═O)Nc | 62.465 | 52.657 | 37.535 | 47.343 | 3.774 | 3.664 | 3.719 |
| | | | | 7 | | (cc3)ccc3F)NC(═CC2═O)C | | | | | | | |
| F17 | N07 | 3465 | P21 | ChemDiv | D513-3628 | c(N(CCOc1ccccc1)S | 48.274 | 33.520 | 51.726 | 66.480 | 5.201 | 5.145 | 5.173 |
| | | | | 7 | | (c2ccccc2)(═O)═O)(nn3 | | | | | | | |
| | | | | | | c4nc(c(Cl)c3C)C)n4 | | | | | | | |
| A7 | C05 | 3467 | N10 | ChemDiv | D664-0047 | C1(═O)N(C)c2c(cc(cc2) | 78.799 | 52.771 | 21.201 | 47.229 | 3.210 | 5.639 | 4.425 |
| | | | | 7 | | CN([H])c3nnnn3CCCC) | | | | | | | |
| | | | | | | N1C | | | | | | | |
| A11 | C09 | 3467 | P15 | ChemDiv | D656-0061 | C(C(═O)N([H])c1ccc | 92.384 | 43.101 | 7.616 | 56.899 | 1.153 | 6.794 | 3.973 |
| | | | | 7 | | (c2c1cccn2)OCC)(Oc(ccc | | | | | | | |
| | | | | | | (c3)CC)c3C4═O)═C4 | | | | | | | |
| A13 | D03 | 3468 | F08 | ChemDiv | D715-2438 | c12c(ccc(O)c10)C3═C | 82.092 | 61.053 | 17.908 | 38.947 | 2.712 | 4.650 | 3.681 |
| | | | | 7 | | (C(═O)O2)CCCC3 | | | | | | | |
| A15 | D05 | 3468 | J09 | ChemDiv | D686-0195 | c1(NC(═O)c2ccc(nc2Cl)C) | 70.121 | 35.542 | 29.879 | 64.458 | 4.525 | 7.696 | 6.110 |
| | | | | 7 | | sc(nc1C(N)═O)Nc3cc | | | | | | | |
| | | | | | | (C)ccc3C | | | | | | | |
| B13 | F03 | 3469 | B20 | ChemDiv | D727-0772 | n1(n2)c(nnc1c3cccc(F) | 57.757 | 87.293 | 42.243 | 12.707 | 6.397 | 1.517 | 3.957 |
| | | | | 7 | | c3)sc2c4c5c([nH]c4) | | | | | | | |
| | | | | | | cccc5 | | | | | | | |
| C17 | H07 | 3469 | F22 | Chem Div | D727-0786 | n1(n2)c(nnc1c3ccoc3C) | 42.718 | 87.278 | 57.282 | 12.722 | 8.674 | 1.519 | 5.097 |
| | | | | 7 | | sc2c4c5c([nH]c4)cccc5 | | | | | | | |
| D7 | I05 | 3469 | H10 | ChemDiv | D727-0717 | n1(c(CCc(c(C)nn2c3ccccc3) | 66.716 | 40.829 | 33.284 | 59.171 | 5.040 | 7.065 | 6.053 |
| | | | | 7 | | c2C)nn4)c4sc(c(cc5) | | | | | | | |
| | | | | | | ccc5N(C)C)n1 | | | | | | | |
| D19 | J09 | 3469 | I21 | ChemDiv | D727-0059 | n12c(nnc1c(cccn3)c3) | 82.773 | 66.747 | 17.227 | 33.253 | 2.609 | 3.970 | 3.290 |
| | | | | 7 | | sc(c4ccccc40C)n2 | | | | | | | |
| E17 | L07 | 3469 | L18 | Chem Div | D727-0768 | n1(n2)c(nnc1CC(C)C) | 79.951 | 47.813 | 20.049 | 52.187 | 3.036 | 6.231 | 4.634 |
| | | | | 7 | | sc2c3c4c([nH]c3)cccc4 | | | | | | | |
| F15 | N05 | 3469 | P09 | Chem Div | D727-0417 | n12c(nnc1c3cccc(F)c3) | 66.674 | 75.341 | 33.326 | 24.659 | 5.046 | 2.944 | 3.995 |
| | | | | 7 | | sc(c(cc4C)c5c(n4)cccc5)n2 | | | | | | | |
| C15 | H05 | 3471 | F20 | ChemDiv | E234-0018 | N1(c2ccc(cc20C)OC) | 61.394 | 77.421 | 38.606 | 22.579 | 4.570 | 2.970 | 3.770 |
| | | | | 7 | | C(═O)c3n(CC1(C)C(═O) | | | | | | | |
| | | | | | | NC4CCCCC4)c5c(cccc5)c3 | | | | | | | |
| D9 | I07 | 3471 | L18 | Chem Div | E234-0006 | C1(C)(Cn(c2c(c3)cccc2) | 42.952 | 72.361 | 57.048 | 27.639 | 6.753 | 3.635 | 5.194 |
| | | | | 7 | | c3C(═O)N1c4cccc(OC)c4) | | | | | | | |
| | | | | | | C(═O)NC5CCCCC5 | | | | | | | |
| D15 | J05 | 3471 | P18 | ChemDiv | E234-0008 | N(Cc1ccccc1)(C2═O)C | 49.411 | 65.899 | 50.589 | 34.101 | 5.989 | 4.485 | 5.237 |
| | | | | 7 | | (C)(Cn(c3c(cccc3)c4)c24) | | | | | | | |
| | | | | | | C(═O)NC5CCCCC5 | | | | | | | |
| E5 | K03 | 3476 | J05 | ChemDiv | E922-0258 | c(cnn1c2ccc(cc2)C)(C | 36.699 | 40.996 | 63.301 | 59.004 | 7.494 | 7.761 | 7.627 |
| | | | | 7 | | (═O)Nc(cc3)ccc3Br)c1 | | | | | | | |
| | | | | | | C(CC4)CCN4 | | | | | | | |
| E11 | K09 | 3479 | J02 | ChemDiv | F083-0285 | N1(c2c(cc(Br)cc2)C | 69.292 | 77.181 | 30.708 | 22.819 | 3.635 | 3.002 | 3.318 |
| | | | | 7 | | (N3)═O)C3═C(SC1═S) | | | | | | | |
| | | | | | | C(NC)═O | | | | | | | |
| F7 | M05 | 3484 | M10 | ChemDiv | F255-0057 | c1(CCc(cc2)ccc2NC | 53.188 | 54.359 | 46.812 | 45.641 | 5.542 | 6.003 | 5.773 |
| | | | | 7 | | (═O)c3ccc(cc3)NC | | | | | | | |
| | | | | | | (C)═O)nc(c4n1c5ccccc5) | | | | | | | |
| | | | | | | cccc4 | | | | | | | |
| F15 | N05 | 3485 | H02 | ChemDiv | F305-0061 | C(CCCN1c2ccc(nn2) | 60.527 | 56.155 | 39.473 | 43.845 | 4.673 | 5.767 | 5.220 |
| | | | | 7 | | c3ccccc3)(C1)C(═O) | | | | | | | |
| | | | | | | N(CCC)CCC | | | | | | | |
| F19 | N09 | 3485 | L21 | ChemDiv | F305-0030 | C(CCCN1c2ccc(nn2) | 57.392 | 64.577 | 42.608 | 35.423 | 5.044 | 4.659 | 4.852 |
| | | | | 7 | | c3ccccc3)(C1)C(═O) | | | | | | | |
| | | | | | | N(CCCC)CC | | | | | | | |
| A11 | C09 | 3474 | H05 | Chem Div | E722-2652 | c12c(CSC(C(NCCCN | 56.478 | 52.173 | 43.522 | 47.827 | 5.564 | 5.937 | 5.750 |
| | | | | 7 | | (CC3)CCC3N4CCCCC4) | | | | | | | |
| | | | | | | ═O)═C1)c5c(CCCC5)s2 | | | | | | | |
| B9 | E07 | 3481 | M15 | Chem Div | F128-0030 | N1(Cc2ccccc2)C(═S)SC | 29.562 | 32.307 | 70.438 | 67.693 | 9.005 | 8.402 | 8.704 |
| | | | | 7 | | (C(N)═O)═C1N | | | | | | | |
| F11 | M09 | 3641 | E19 | NCC1- | SAM001246816 | Nc1nc(cs1)C(═NOCC | 62.883 | 78.109 | 37.117 | 21.891 | 4.745 | 2.717 | 3.731 |
| | | | | 2014 | | (═O)O)C(═O)N[C@H]2 | | | | | | | |
| | | | | | | [C@H]3SCC(═C | | | | | | | |
| | | | | | | (N3C2═O)C(═O)O) | | | | | | | |
| | | | | | | C═C•O | | | | | | | |
| E7 | 1 - | 3495 | J12 | ChemDiv | | | 76.845 | 77.278 | 23.155 | 22.722 | 4.893 | 5.513 | 5.203 |
| | K05 | | | 7 | | | | | | | | | |
| F13 | 1 - | 3493 | H07 | ChemDiv | | | 56.501 | 46.007 | 43.499 | 53.993 | 9.192 | 13.100 | 11.146 |
| | N03 | | | 7 | | | | | | | | | |
| I19 | 2 - | 3533 | B03 | ChemDiv | | | 86.387 | 82.990 | 13.613 | 17.010 | 2.877 | 4.127 | 3.502 |
| | H09 | | | 7 | | | | | | | | | |
| J15 | 2 - | 3519 | J06 | ChemDiv | | | 74.648 | 73.024 | 25.352 | 26.976 | 5.357 | 6.545 | 5.951 |
| | J05 | | | 7 | | | | | | | | | |
| K9 | 2 - | 3526 | I11 | ChemDiv | | | 86.517 | 82.925 | 13.483 | 17.075 | 2.849 | 4.143 | 3.496 |
| | K07 | | | 7 | | | | | | | | | |
| A7 | C05 | 1725 | P16 | Enamine | | | 51.002 | 55.586 | 48.998 | 44.414 | 13.818 | 12.379 | 13.098 |
| | | | | 2 | | | | | | | | | |
| B15 | D06 | 1736 | A09 | Enamine | | | 38.494 | 33.210 | 61.506 | 66.790 | 17.345 | 18.616 | 17.980 |
| | | | | 2 | | | | | | | | | |
| B19 | D08 | 1748 | D11 | Enamine | | | 89.426 | 90.096 | 10.574 | 9.904 | 2.982 | 2.760 | 2.871 |
| | | | | 2 | | | | | | | | | |
| D13 | E12 | 1752 | N03 | Enamine | | | 84.563 | 84.106 | 15.437 | 15.894 | 4.353 | 4.430 | 4.392 |
| | | | | 2 | | | | | | | | | |
| D17 | E14 | 1753 | O14 | Enamine | | | 68.459 | 61.525 | 31.541 | 38.475 | 8.895 | 10.724 | 9.809 |
| | | | | 2 | | | | | | | | | |
| G7 | G13 | 1753 | H15 | Enamine | | | 88.094 | 90.063 | 11.906 | 9.937 | 3.358 | 2.770 | 3.064 |
| | | | | 2 | | | | | | | | | |
| G9 | G14 | 1753 | P07 | Enamine | | | 73.728 | 62.252 | 26.272 | 37.748 | 7.409 | 10.521 | 8.965 |
| | | | | 2 | | | | | | | | | |
| G13 | H03 | 1718 | I07 | Enamine | | | 56.423 | 66.997 | 43.577 | 33.003 | 12.289 | 9.199 | 10.744 |
| | | | | 2 | | | | | | | | | |
| M5 | L07 | 1747 | P17 | Enamine | | | 89.157 | 84.899 | 10.843 | 15.101 | 3.058 | 4.209 | 3.633 |
| | | | | 2 | | | | | | | | | |
| N21 | M12 | 1753 | D11 | Enamine | | | 53.612 | 51.308 | 46.388 | 48.692 | 13.082 | 13.571 | 13.327 |
| | | | | 2 | | | | | | | | | |
| O7 | M15 | 1755 | C16 | Enamine | | | 1.585 | −0.759 | 98.415 | 100.759 | 27.754 | 28.083 | 27.919 |
| | | | | 2 | | | | | | | | | |
| O13 | N05 | 1734 | G14 | Enamine | | | 75.246 | 29.310 | 24.754 | 70.690 | 6.981 | 19.703 | 13.342 |
| | | | | 2 | | | | | | | | | |
| P5 | N11 | 1752 | G02 | Enamine | | | 72.766 | 67.793 | 27.234 | 32.207 | 7.680 | 8.977 | 8.328 |
| | | | | 2 | | | | | | | | | |
| P7 | N12 | 1753 | D13 | Enamine | | | 67.759 | 75.295 | 32.241 | 24.705 | 9.092 | 6.886 | 7.989 |
| | | | | 2 | | | | | | | | | |
| P9 | N13 | 1753 | M10 | Enamine | | | 47.564 | 33.546 | 52.436 | 66.454 | 14.787 | 18.522 | 16.655 |
| | | | | 2 | | | | | | | | | |
| B3 | C13 | 1753 | E12 | Enamine | | | 83.281 | 84.801 | 16.719 | 15.199 | 4.715 | 4.236 | 4.476 |
| | | | | 2 | | | | | | | | | |
| C3 | D10 | 1749 | L05 | Enamine | | | 68.482 | 88.171 | 31.518 | 11.829 | 8.888 | 3.297 | 6.093 |
| | | | | 2 | | | | | | | | | |
| F21 | G10 | 1749 | P06 | Enamine | | | 77.415 | 62.113 | 22.585 | 37.887 | 6.369 | 10.560 | 8.465 |
| | | | | 2 | | | | | | | | | |
| F3 | F14 | 1753 | O20 | Enamine | | | 85.206 | 87.182 | 14.794 | 12.818 | 4.172 | 3.573 | 3.872 |
| | | | | 2 | | | | | | | | | |
| K3 | J12 | 1753 | B05 | Enamine | | | 65.183 | 64.815 | 34.817 | 35.185 | 9.819 | 9.807 | 9.813 |
| | | | | 2 | | | | | | | | | |
| A5 | C04 | 1755 | N22 | Enamine | | | 78.440 | 82.300 | 21.560 | 17.700 | 4.715 | 4.801 | 4.758 |
| | | | | 2 | | | | | | | | | |
| E13 | F09 | 1758 | G06 | Enamine | | | 83.149 | 74.293 | 16.851 | 25.707 | 3.685 | 6.973 | 5.329 |
| | | | | 2 | | | | | | | | | |
| F19 | G09 | 1758 | K20 | Enamine | | | 83.546 | 78.923 | 16.454 | 21.077 | 3.598 | 5.717 | 4.658 |
| | | | | 2 | | | | | | | | | |
| H19 | I03 | 1755 | I12 | Enamine | | | 82.759 | 82.348 | 17.241 | 17.652 | 3.770 | 4.788 | 4.279 |
| | | | | 2 | | | | | | | | | |
| I11 | I09 | 1758 | N22 | Enamine | | | 65.000 | 43.436 | 35.000 | 56.564 | 7.653 | 15.344 | 11.499 |
| | | | | 2 | | | | | | | | | |
| I19 | I13 | 1761 | E07 | Enamine | | | 85.202 | 82.585 | 14.798 | 17.415 | 3.236 | 4.724 | 3.980 |
| | | | | 2 | | | | | | | | | |
| M19 | L14 | 1762 | A18 | Enamine | | | 83.569 | 88.418 | 16.431 | 11.582 | 3.593 | 3.142 | 3.367 |
| | | | | 2 | | | | | | | | | |
| E3 | F04 | 1755 | P16 | Enamine | | | 86.207 | 86.295 | 13.793 | 13.705 | 3.016 | 3.718 | 3.367 |
| | | | | 2 | | | | | | | | | |
| I3 | I05 | 1756 | I06 | Enamine | | | 85.839 | 72.729 | 14.161 | 27.271 | 3.096 | 7.398 | 5.247 |
| | | | | 2 | | | | | | | | | |
| L3 | K09 | 1758 | O19 | Enamine | | | 76.844 | 77.038 | 23.156 | 22.962 | 5.063 | 6.229 | 5.646 |
| | | | | 2 | | | | | | | | | |
| M3 | L06 | 1756 | P13 | Enamine | | | 65.805 | 56.289 | 34.195 | 43.711 | 7.477 | 11.857 | 9.667 |
| | | | | 2 | | | | | | | | | |
| A5 | C04 | 1763 | I12 | Enamine | | | 83.842 | 90.811 | 16.158 | 9.189 | 5.267 | 3.801 | 4.534 |
| | | | | 2 | | | | | | | | | |
| A9 | C06 | 1766 | A21 | Enamine | | | 88.274 | 93.079 | 11.726 | 6.921 | 3.822 | 2.863 | 3.343 |
| | | | | 2 | | | | | | | | | |
| B7 | C15 | 1778 | O10 | Enamine | | | 64.430 | 88.268 | 35.570 | 11.732 | 11.596 | 4.853 | 8.225 |
| | | | | 2 | | | | | | | | | |
| C5 | D11 | 1771 | C01 | Enamine | | | 88.170 | 85.805 | 11.830 | 14.195 | 3.856 | 5.873 | 4.864 |
| | | | | 2 | | | | | | | | | |
| C11 | D14 | 1776 | K03 | Enamine | | | 79.166 | 89.979 | 20.834 | 10.021 | 6.792 | 4.146 | 5.469 |
| | | | | 2 | | | | | | | | | |
| E17 | F11 | 1771 | G07 | Enamine | | | 91.027 | 90.967 | 8.973 | 9.033 | 2.925 | 3.737 | 3.331 |
| | | | | 2 | | | | | | | | | |
| F19 | G09 | 1767 | N18 | Enamine | | | 89.203 | 77.279 | 10.797 | 22.721 | 3.520 | 9.399 | 6.460 |
| | | | | 2 | | | | | | | | | |
| G5 | G12 | 1773 | C12 | Enamine | | | 89.162 | 86.559 | 10.838 | 13.441 | 3.533 | 5.561 | 4.547 |
| | | | | 2 | | | | | | | | | |
| G9 | G14 | 1776 | N07 | Enamine | | | 89.496 | 93.395 | 10.504 | 6.605 | 3.424 | 2.733 | 3.078 |
| | | | | 2 | | | | | | | | | |
| I21 | I14 | 1776 | O13 | Enamine | | | 45.837 | 64.293 | 54.163 | 35.707 | 17.657 | 14.772 | 16.214 |
| | | | | 2 | | | | | | | | | |
| A3 | C03 | 1762 | P20 | Enamine | | | 80.922 | 78.623 | 19.078 | 21.377 | 6.219 | 8.843 | 7.531 |
| | | | | 2 | | | | | | | | | |
| D21 | F03 | 1763 | C05 | Enamine | | | 79.742 | 74.804 | 20.258 | 25.196 | 6.604 | 10.423 | 8.514 |
| | | | | 2 | | | | | | | | | |
| E3 | F04 | 1763 | L12 | Enamine | | | 90.235 | 88.285 | 9.765 | 11.715 | 3.183 | 4.846 | 4.015 |
| | | | | 2 | | | | | | | | | |
| F3 | F14 | 1776 | N02 | Enamine | | | 81.004 | 83.026 | 18.996 | 16.974 | 6.193 | 7.022 | 6.607 |
| | | | | 2 | | | | | | | | | |
| G3 | G11 | 1771 | H02 | Enamine | | | 87.516 | 83.869 | 12.484 | 16.131 | 4.070 | 6.673 | 5.372 |
| | | | | 2 | | | | | | | | | |
| J3 | I15 | 1779 | M04 | Enamine | | | 50.702 | 51.848 | 49.298 | 48.152 | 16.071 | 19.920 | 17.995 |
| | | | | 2 | | | | | | | | | |
| K3 | J12 | 1773 | L13 | Enamine | | | 61.850 | 60.963 | 38.150 | 39.037 | 12.436 | 16.149 | 14.293 |
| | | | | 2 | | | | | | | | | |
| L3 | K09 | 1768 | I12 | Enamine | | | 89.285 | 85.891 | 10.715 | 14.109 | 3.493 | 5.837 | 4.665 |
| | | | | 2 | | | | | | | | | |
| O3 | M13 | 1775 | O19 | Enamine | | | 80.140 | 74.346 | 19.860 | 25.654 | 6.474 | 10.613 | 8.543 |
| | | | | 2 | | | | | | | | | |

SaCas9 inhibitors may comprise a compound according to the general formula:

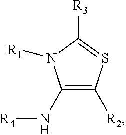

wherein X is selected from N or S, R₁ can be selected from

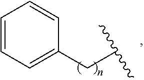

wherein n is 0 to 5 and is optionally substituted, in some embodiments, the ring is a benzyl ring. In any embodiment, the ring can be substituted at one or more locations on the ring with hydroxyl, alkoxy, phenyl, halogen, CF₃, amine, amide, saturated or unsaturated hydrocarbons optionally forming a 3, 4, 5, 6, 7, or 8 membered ring, wherein R₂ is C(O)NH₂, C(O)OR₅, CN, C(O)NHR, wherein R₃ is S or O; wherein R₄ is H, alkoxy, saturate or unsaturated hydrocarbons optionally forming a 3, 4, 5, 6, 7 or 8 membered ring with R₁; wherein R₅ is independently hydrogen, alkyl, alkoxy, hydroxyl, alkylenyl, alkynyl, heterocyclyl, heteroalkyl, or heteroaryl.

SaCas9 inhibitors may comprise a compound according to the general formula:

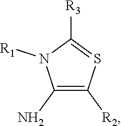

wherein R₂ is C(O)NH₂, C(O)OR, CN, C(O)NHR, wherein R is independently hydrogen, alkyl, alkoxy, hydroxyl, alkylenyl, alkynyl, heterocyclyl, heteroalkyl, or heteroaryl. In certain embodiments, when R₂ is C(O)NH₂, R₁ can be selected from

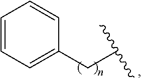

wherein n is 0 to 5 and is optionally substituted, in some embodiments, the ring is a benzyl ring. In any embodiment, the ring can be substituted at one or more locations on the ring with hydroxyl, alkoxy, phenyl, halogen, CF₃, amine, amide, saturated or unsaturated hydrocarbons optionally forming a 3, 4, 5, 6, 7, or 8 membered ring. In particular embodiments, the SaCas9 inhibitor is according to any one of compounds 1-28 of Table 6.

TABLE 6
SaCas9 Inhibitors

| 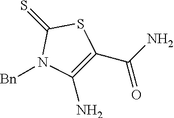 [SMILES: NC(=O)c1sc(=S)n(Cc2ccccc2)c1N] | 1 |
|---|---|
| 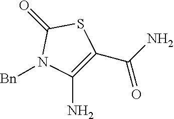 [SMILES: NC(=O)c1sc(=O)n(Cc2ccccc2)c1N] | 2 |
| 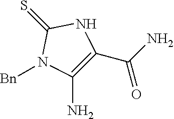 [SMILES: NC(=O)c1[nH]c(=S)n(Cc2ccccc2)c1N] | 3 |
| 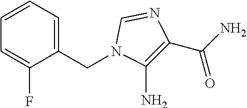 [SMILES: NC(=O)c1ncn(Cc2ccccc2F)c1N] | 4 |
| 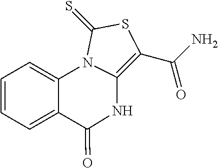 [SMILES: NC(=O)c1sc(=S)n2c1[nH]c(=O)c1ccccc12] | 5 |
| 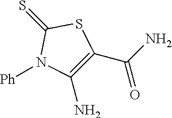 [SMILES: NC(=O)c1sc(=S)n(-c2ccccc2)c1N] | 6 |
| 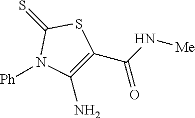 [SMILES: CNC(=O)c1sc(=S)n(-c2ccccc2)c1N] | 7 |
| 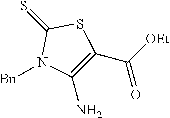 [SMILES: CCOC(=O)c1sc(=S)n(Cc2ccccc2)c1N] | 8 |
| 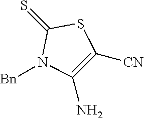 [SMILES: N#Cc1sc(=S)n(Cc2ccccc2)c1N] | 9 |
| 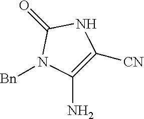 [SMILES: N#Cc1[nH]c(=O)n(Cc2ccccc2)c1N] | 10 |
| R² = C(O)NH₂, R¹ = | |
| 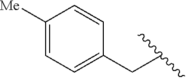 [SMILES: *Cc1ccc(C)cc1] | 11 |
| 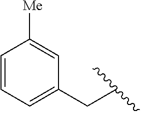 [SMILES: *Cc1cccc(C)c1] | 12 |
| 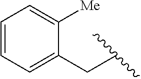 [SMILES: *Cc1ccccc1C] | 13 |
| 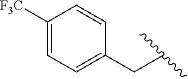 [SMILES: *Cc1ccc(C(F)(F)F)cc1] | 14 |
| 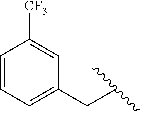 [SMILES: *Cc1cccc(C(F)(F)F)c1] | 15 |
| 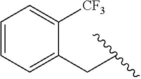 [SMILES: *Cc1ccccc1C(F)(F)F] | 16 |
| 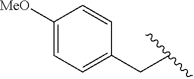 [SMILES: *Cc1ccc(OC)cc1] | 17 |
| 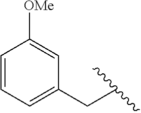 [SMILES: *Cc1cccc(OC)c1] | 18 |
| 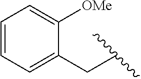 [SMILES: *Cc1ccccc1OC] | 19 |
| 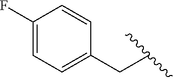 [SMILES: *Cc1ccc(F)cc1] | 20 |
| 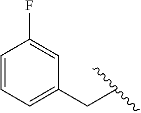 [SMILES: *Cc1cccc(F)c1] | 21 |
| 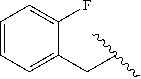 [SMILES: *Cc1ccccc1F] | 22 |
| 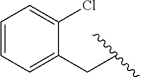 [SMILES: *Cc1ccccc1Cl] | 23 |
| 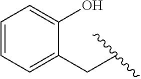 [SMILES: *Cc1ccccc1O] | 24 |
| 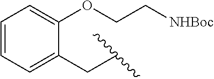 [SMILES: *Cc1ccccc1OCCNC(=O)OC(C)(C)C] | 25 |
| 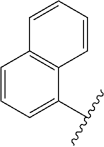 [SMILES: *c1cccc2ccccc12] | 26 |
| 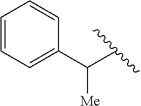 [SMILES: *C(C)c1ccccc1] | 27 |
| 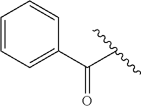 [SMILES: *C(=O)c1ccccc1] | 28 |

### FnCpf1 Inhibitors

Over 119,000 compounds have been screened as potential inhibitors of FnCpf1. Results at or above 3 sigma hits/total compounds were 263/119,362 (0.22% hit rate). 263/263 cherry picks were tested.

Libraries tested included: Torcis Bioactive (1,120/1,120) with a Hit rate: 0.36% (4 compounds); ChemDiv2 (8,544/8,544) with a Hit rate: 0.023% (2 compounds total); ChemDiv6 (7,040/44,000) with a Hit rate: 0.085% (6 compounds so far, prioritizing other libraries first); ChemDiv7 (49,128/49,128) with a Hit rate: 0.16% (78 compounds total); Enamine2+Enamine2a (26,576/26,576) with a Hit rate: 0.20% (52 compounds total); Asinex 2 (23,031/23,031; Rachit's screening) with a Hit rate: 0.30% (70 compounds); Asinex 3 (3,923/3,923; Rachit's screening) with a Hit rate: 1.2% (47 compounds total, most from just one plate).

Additional compounds were selected from a few libraries after lowering the hit cutoff rate to 2.8 sigma. Torcis Bioactive (1,120/1,120) 2.8-3s hits: 2 additional compounds). ChemDiv2 (8,544/8,544)—2.8-3s hits: 1 additional compound); ChemDiv6 (7,040/44,000)—2.8-3 s hits: 1 additional compound); ChemDiv7 (49,128/49,128)—2.8-3 s hits: 22 additional compounds); Enamine2+Enamine2a (26,576/26,576) 2.8-3s hits: 16 additional compounds). 2.8 sigma hits/total compounds: 305/119,362 (0.255% hit rate). The additional 42 cherry picks+4 additional 3s hits overlooked in the first run were ordered.

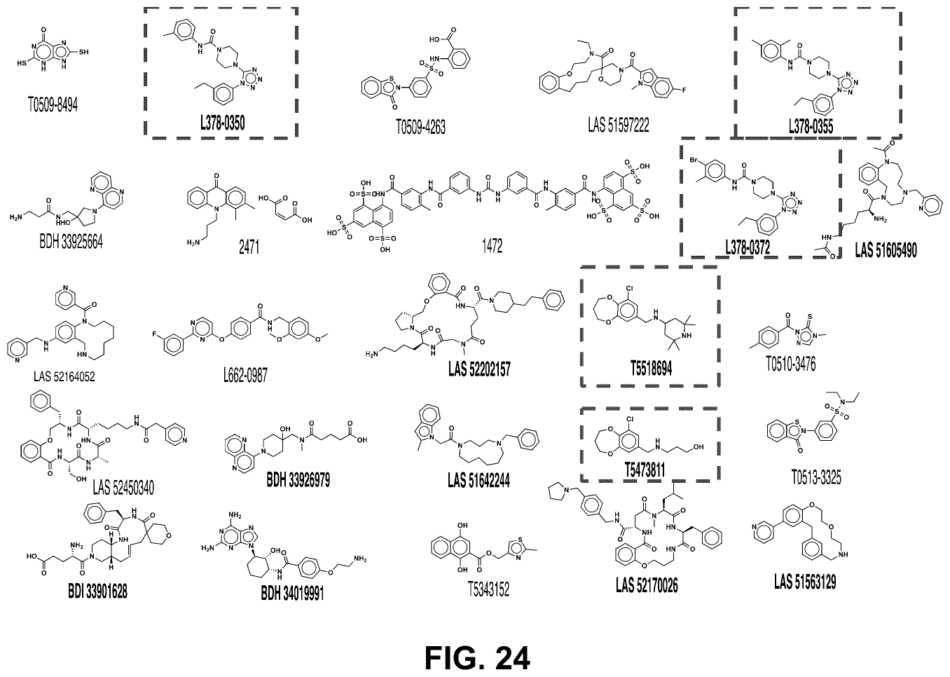

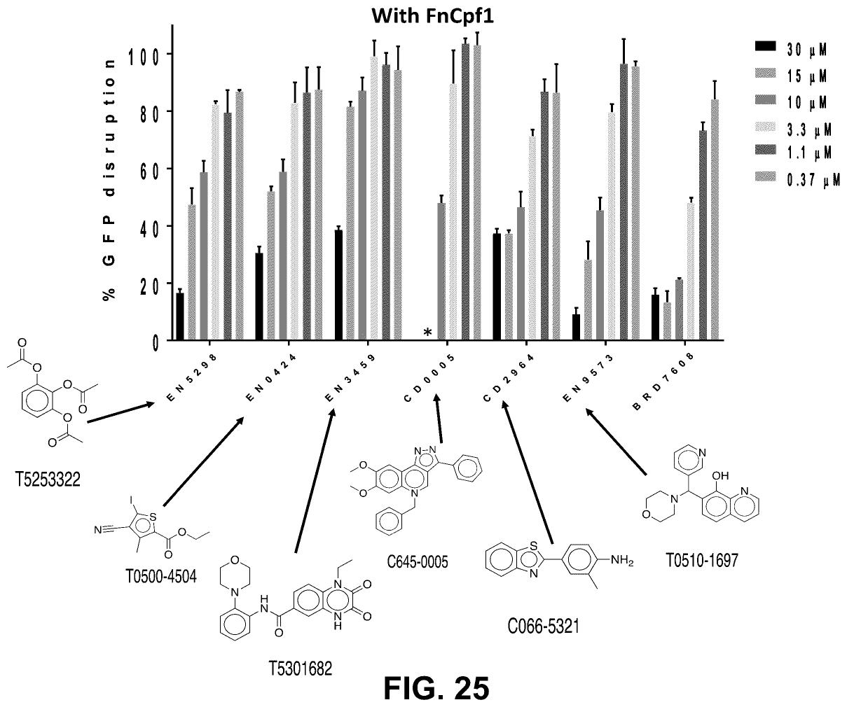

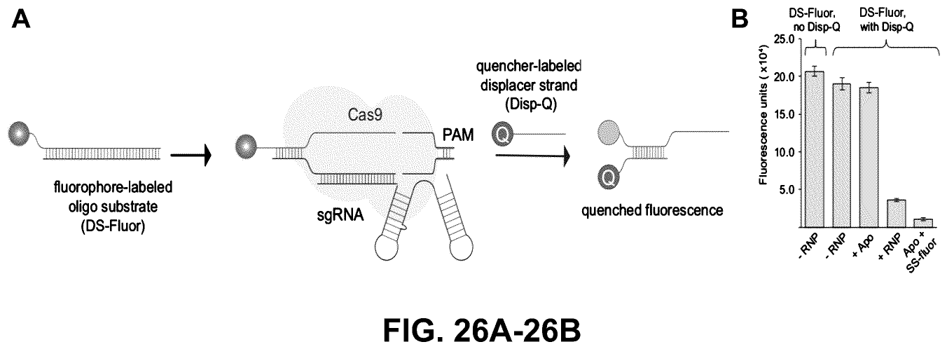

Keeping all compounds with FnCpf1 activity <80% (25); with prioritization of all compounds with Nde1 activity >80% (13) (bolded) (FIG. 24). All ChemDiv libraries from ChemDiv, Inc., San Diego, CA; all Enamine libraries, ENAMINE Ltd., Kiev, Ukraine. Compound reference numbers supplied in each of the examples can be used to identify compounds in these commercially available libraries.

Particular compounds of interest as inhibitors of FnCpf1 have been identified, included below:

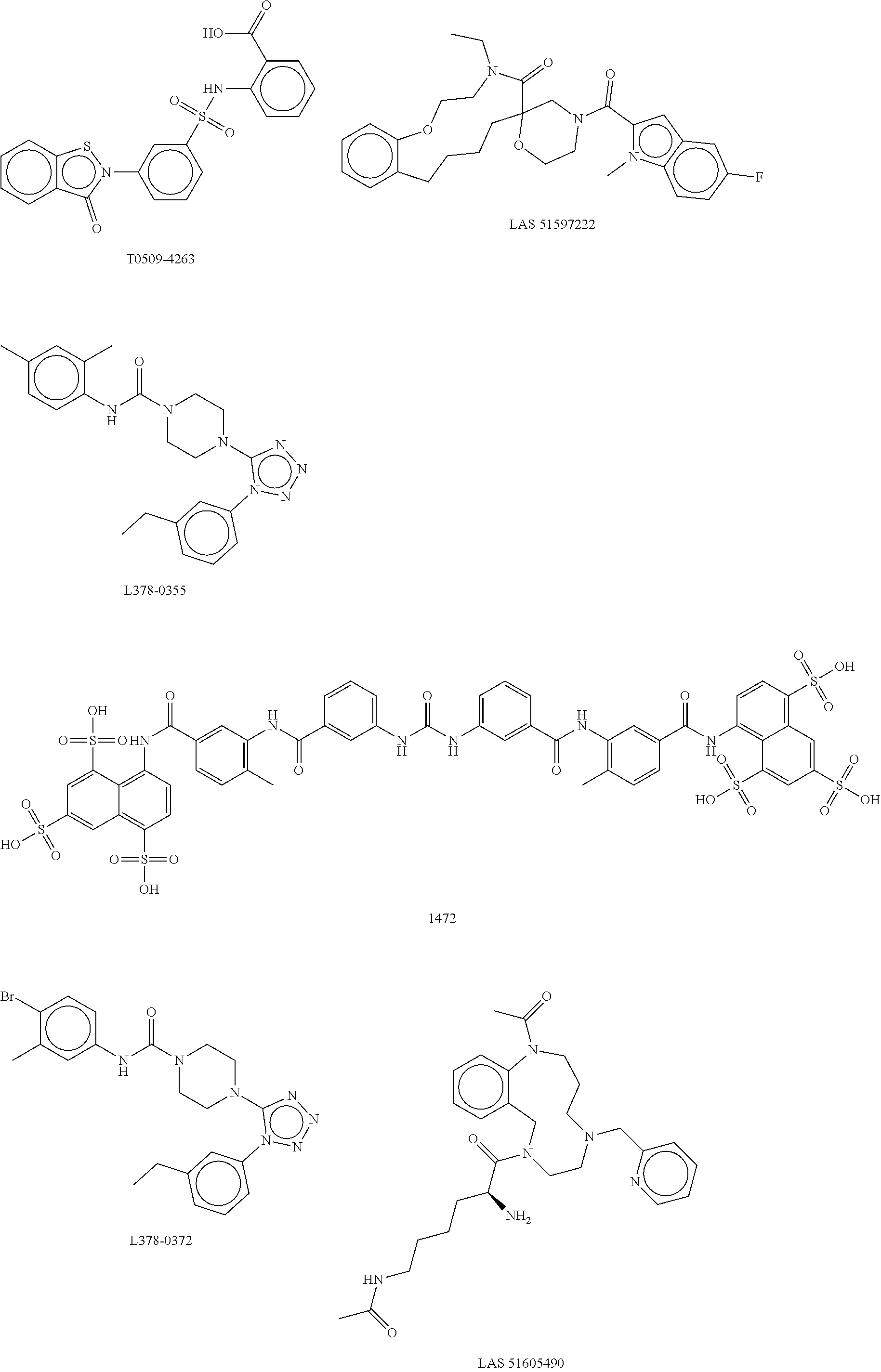

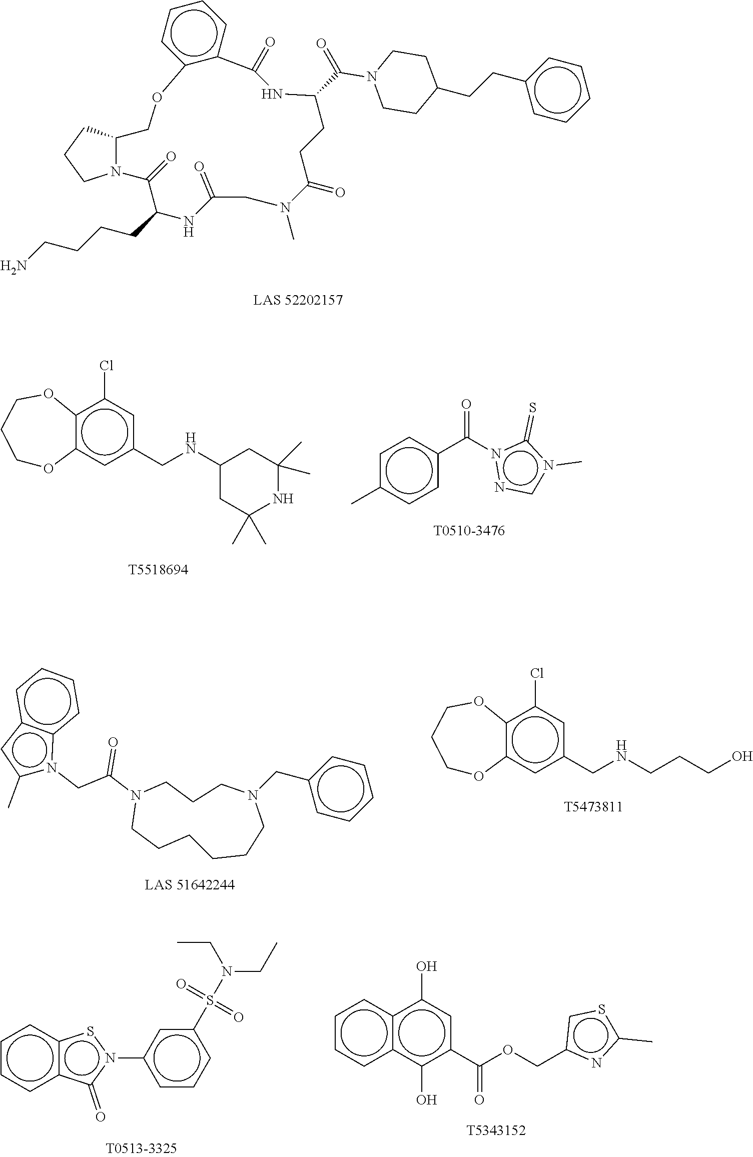

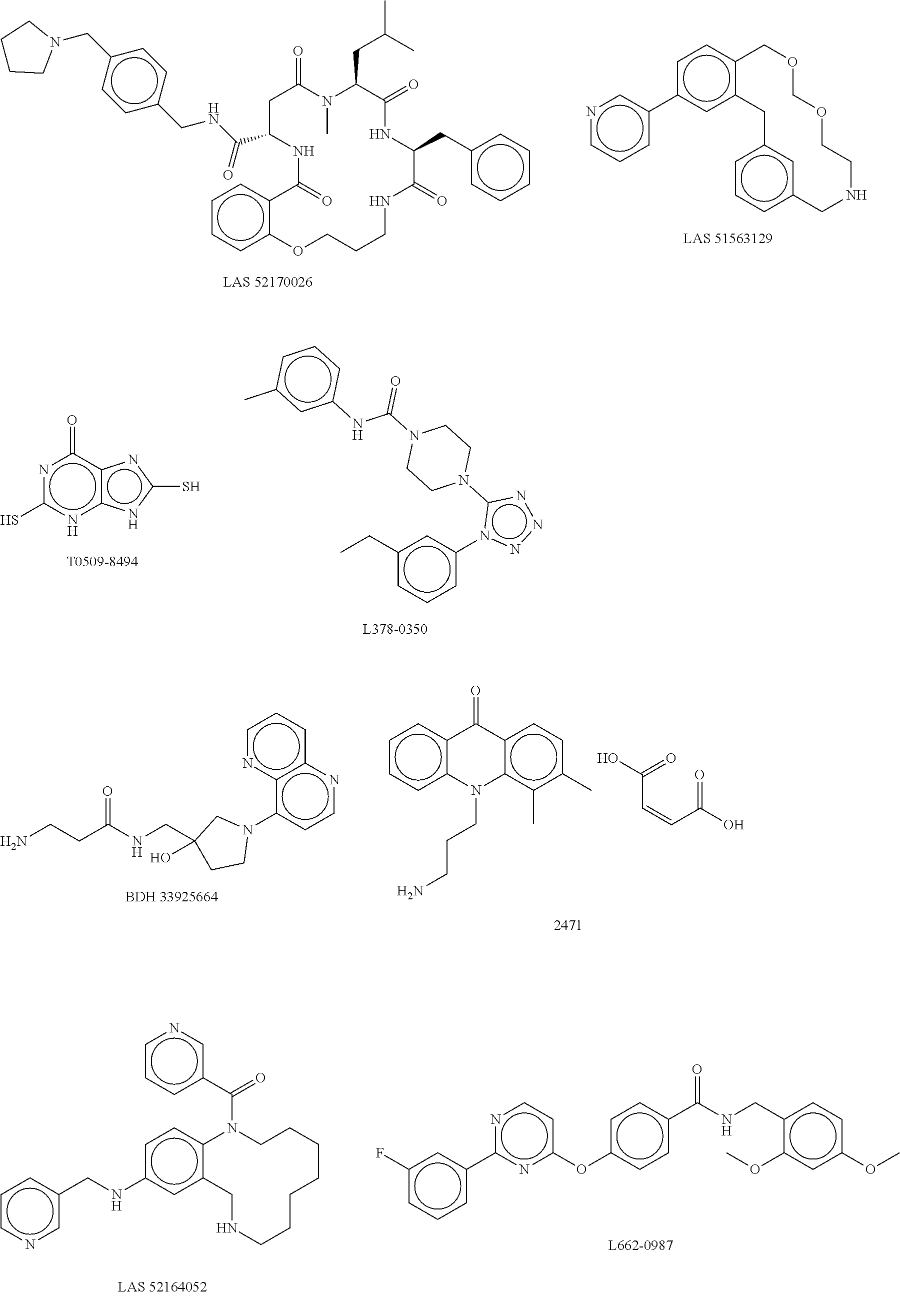

In embodiments, the inhibitor is selected from

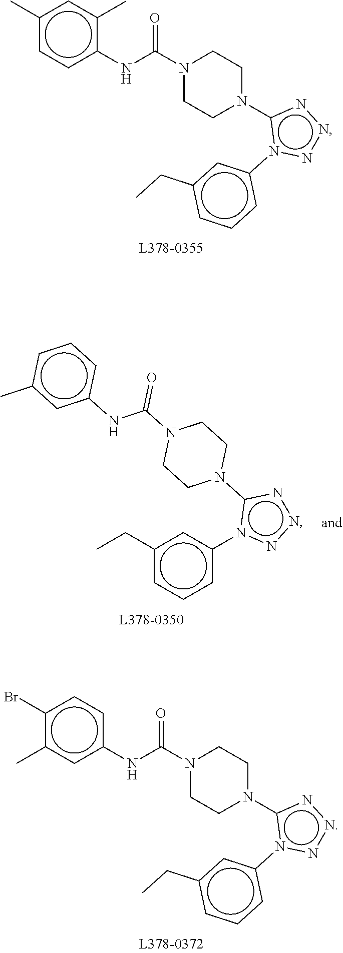

### Example 2

### Introduction

CRISPR-associated nucleases (e.g., SpCas9, SaCas9, Cas12) are programmable RNA-guided endonucleases used to induce site-specific DNA strand breaks, though their non-specific or excessive activity can have deleterious consequences.¹⁻⁵ As the specificity of such strand breaks can depend on extrinsic factors, such as nuclease concentration and activity duration, the need to control these factors has propelled the discovery of anti-CRISPR molecules that can fine-tune the nuclease activity over dose and time.⁶⁻⁸ Ideal anti-CRISPR molecules should be: (1) cell-permeable for facile delivery, precise dosing, and temporal control of the nuclease activity, (2) non-immunogenic and stable in circulation for in vivo use, (3) fast-acting to ensure rapid modulation of nuclease activity and specificity, and (4) easy to use and inexpensive. The precision control of intracellular enzymes is nearly always accomplished using small molecules, which generally possess these desired attributes.⁹⁻¹¹ However, the identification of small-molecule inhibitors of CRISPR-associated nucleases requires a suite of robust, high-throughput, orthogonal, sensitive, and inexpensive activity assays, which are currently unavailable. It is challenging to develop such assays because these nucleases operate via different mechanisms^{12, 13} and their tight binding to DNA yields a single turnover enzyme, preventing signal amplification via multiple catalytic cycles.^{14, 15} Additionally, Cas nucleases possess two nuclease domains that would need to be inactivated and are DNA-binding proteins^{12,16} that are often deemed chemically intractable. Finally, novel protein folds and massive conformational changes during the catalytic cycle complicate rational, structure-guided design approaches.^{12,15,16}

Previously, Applicants developed an assay probing small molecules that disrupt the SpCas9/protospacer adjacent motif(PAM) interaction and discovered BRD0539, a first-generation SpCas9 inhibitor.¹⁷ This small-molecule screening assay based on PAM recognition by SpCas9, the initial step in the catalytic process, overlooks other modes of inhibition (e.g., nuclease activity) and requires high concentrations of SpCas9:gRNA complex, both of which lower the chances of inhibitor discovery. Historically, assays disrupting protein:DNA interactions have not furnished potent small-molecule inhibitors.¹⁸ Additionally, different Cas nucleases recognize different PAM sequences, and preventing this assay from being generalizable to other Cas9 orthologs. Despite examining ˜1000 analogs, BRD0539 had a poor potency, was unable to enhance SpCas9 specificity, and its inhibitory activity depended on the genomic loci or mode of SpCas9 delivery (e.g., plasmid or as a ribonucleoprotein complex). Finally, the synthesis of BRD0539 is cumbersome (8 steps from commercially available materials) and low-yielding, which prohibits its optimization and large-scale production.¹⁷

Applicants hypothesized that small-molecule screening using an assay that cumulatively reports all steps of the catalytic cycle could furnish improved inhibitors. Herein, Applicants describe such a fluorescence resonance energy transfer (FRET)-based cumulative activity assay (CAA) that reports on all the catalytic activity steps, requires 10-fold less SpCas9:gRNA complex compared to the PAM-binding assay, and is broadly applicable across CRISPR nuclease families. Leveraging CAA's high-throughput nature, Applicants screened 122,409 small-molecules, followed by triaging with a suite of orthogonal cellular secondary assays. Using this pipeline, Applicants discovered BRD7586, which is ˜2-fold more potent than BRD0539 and inhibits SpCas9 at multiple genomic loci irrespective of the mode of SpCas9 delivery. Applicants demonstrate that BRD7586 specifically engages SpCas9 but not Cas12a in cells, and it enhances SpCas9 specificity at multiple loci. With a molecular weight of 408 Da, BRD7586 is the smallest known anti-CRISPR and can be synthesized on a large scale in a single step from the commercially available starting materials. Finally, based on structure-activity relationship studies, Applicants have identified an inactive analog of BRD7586. Overall, Applicants present a general, inexpensive, high-throughput and ready-to-implement suite of assays to rapidly identify synthetic, miniature, and cell-permeable inhibitors of CRISPR-associated nucleases and demonstrate the utility of the identified inhibitors to improve genome editing specificity.

### Results

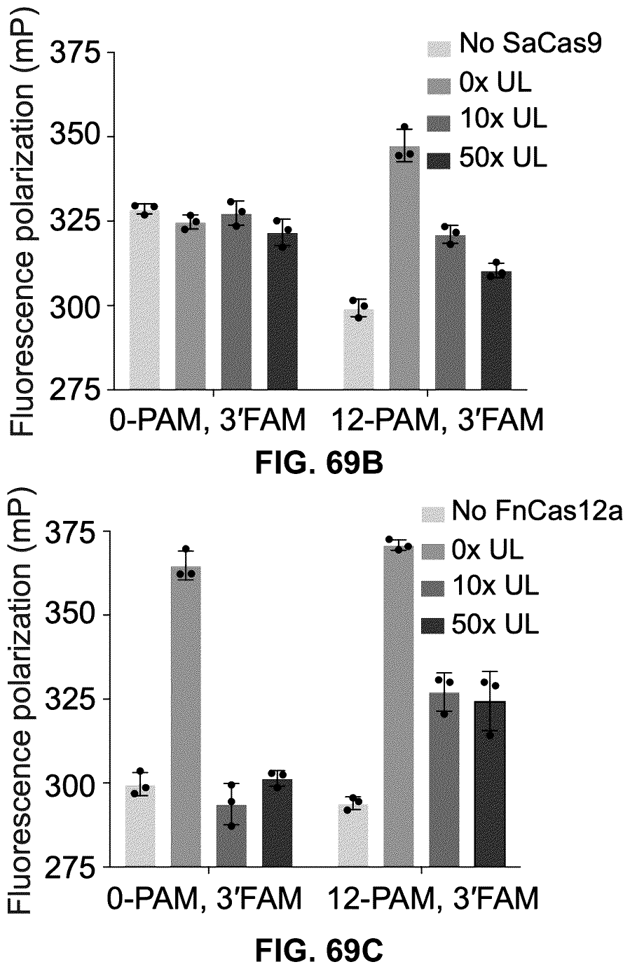

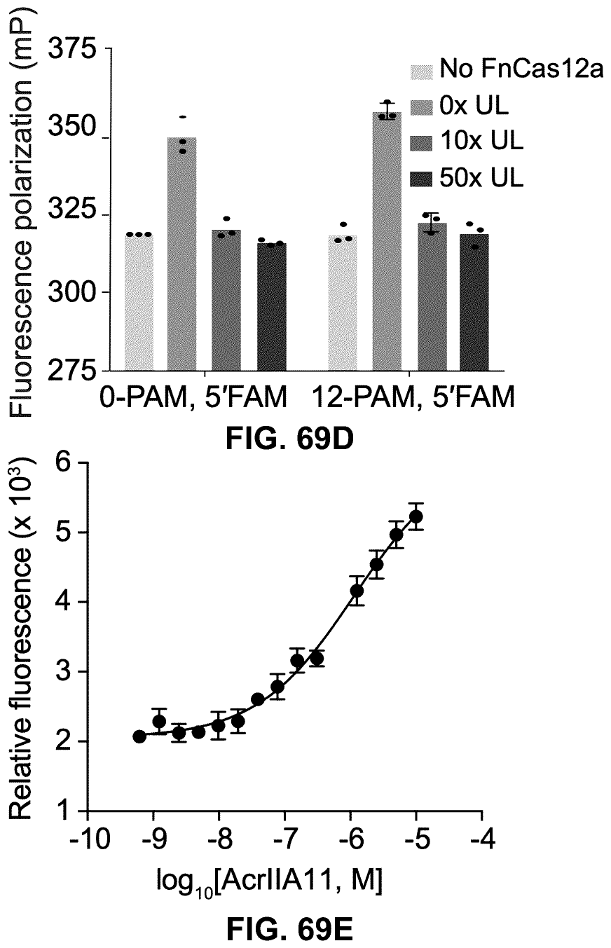

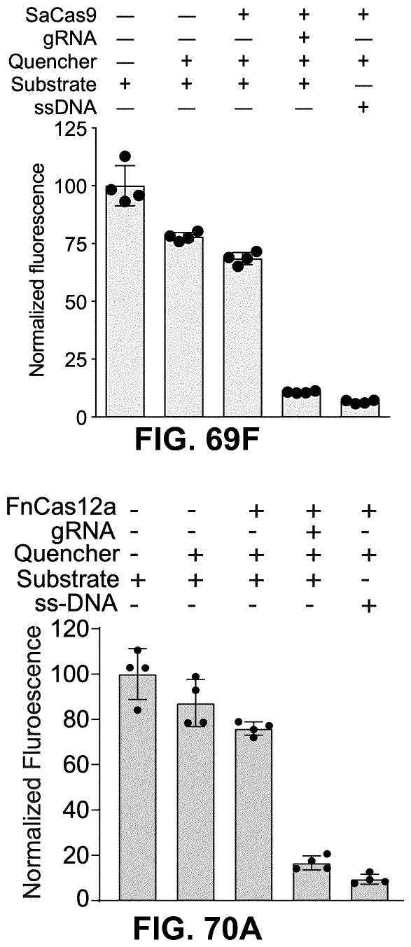

Development of cumulative activity assay (CAA) for SpCas9. Applicants previously reported an assay that uses fluorescence polarization (FP) to monitor the binding between a fluorophore-labeled poly-PAM DNA oligonucleotide and SpCas9 charged with a non-targeting gRNA.¹⁷ This assay permitted screening for small molecules that interfered with the early steps of the SpCas9 catalytic mechanism, namely, binding of SpCas9 and the relatively low-affinity NGG PAM DNA sequence. However, this assay failed to identify molecules that block the cutting activity of SpCas9's nuclease domains and could not be applied for Cas12a as the enzyme bound to the DNA in a PAM-independent fashion (FIG. 69A-D). A high-throughput assay to monitor the nuclease activity of SpCas9 was reported but did not produce ideal chemical matter in terms of toxicity, potency, and on-target specificity.¹⁹ To address these issues, Applicants sought to develop an assay that cumulatively reports on all steps in the catalytic cycle of these nucleases.

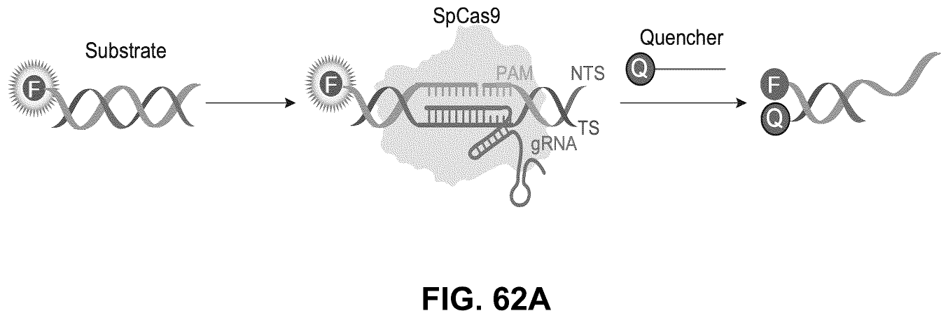

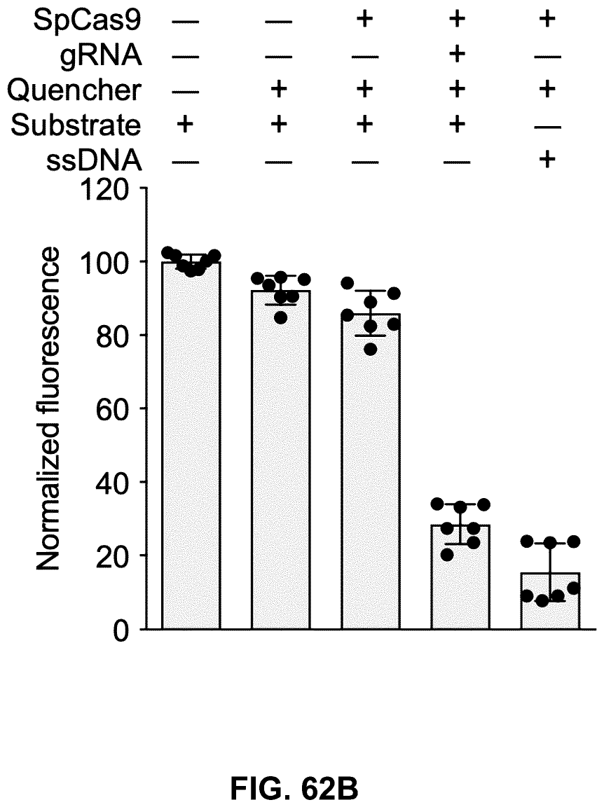

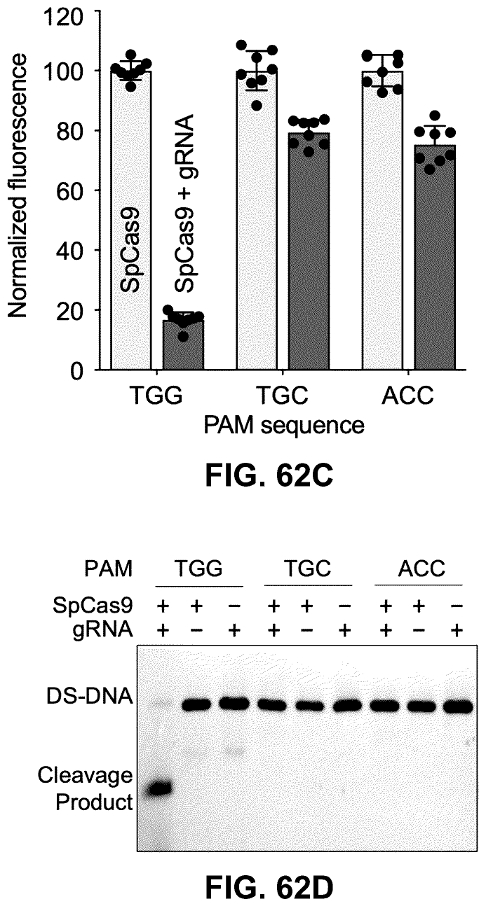

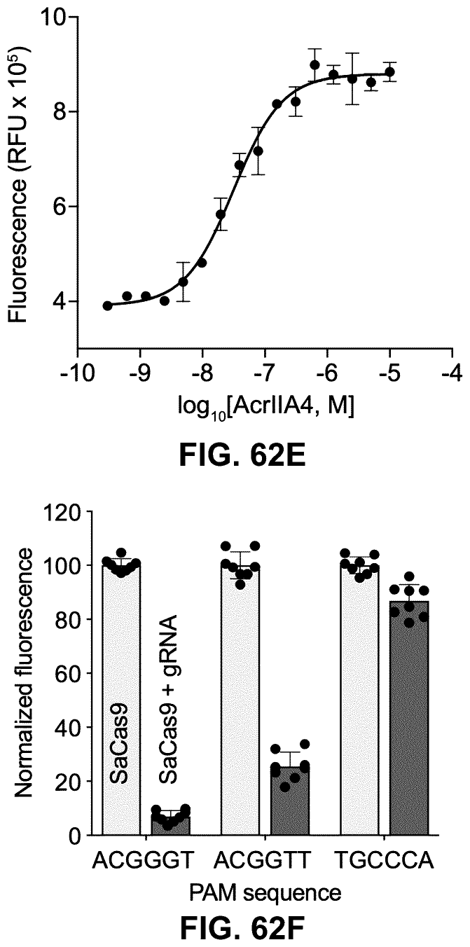

Applicants based the assay on the observation that while Cas9 is bound to the DNA substrate following the double-strand break, the 5′ distal non-target DNA strand is only weakly held by Cas9, and this strand can be displaced upon addition of excess complementary single-stranded DNA (ss-DNA),¹⁴ analogous to toe-hold-mediated strand displacement.^{20, 21} Therefore, Applicants designed a FRET-based assay wherein the 5′ end of the non-target strand in the substrate was labeled with a fluorophore. The 3′ end of the displacing single-stranded DNA was labeled with a quencher. Following nuclease cleavage of the substrate, the 3′-labeled quenching DNA strand (present in excess) could outcompete the weakly held 3′ strand to anneal to the 5′ strand. The resulting FRET fluorescence quenching provides an optical readout for nuclease activity (FIG. 62A).

When testing this CAA, loss of fluorescence was indeed only observed when active SpCas9:gRNA was added to a mixture of both the substrate and quencher. When all components were present, the quenching efficiency was similar to the control when the quencher was directly added to the complementary fluorophore-labeled single-strand oligonucleotide (FIG. 62B). Applicants validated that the loss of fluorescence depended on the presence of an NGG PAM (FIG. 62C) and confirmed the activity correlation between the CAA and that observed using gel electrophoresis on the same assay (FIG. 62D). Importantly, CAA can recapitulate concentration-dependent inhibition of SpCas9 by anti-CRISPR proteins that operate by different mechanisms (FIG. 62E and FIG. 69E). For example, AcrIIA4 disrupts the PAM recognition by SpCas9:gRNA complex while AcrIIA11 inhibits DNA cleavage by trapping SpCas9:gRNA at the PAM-rich sites.^{7 22-25} However, CAA cannot distinguish between various inhibitory mechanisms, for which additional assays will be required.

Generalization of CAA to other Cas nucleases. Applicants sought to generalize CAA to other CRISPR-associated nucleases, including from Staphylococcus aureus (SaCas9). Given the similarities between SaCas9 and SpCas9 modes of DNA-substrate binding and protein folding,^{26, 27} Applicants hypothesized that SaCas9-induced strand displacement could be similarly measured. Indeed, fluorescence quenching correlated with substrate cleavage in the CAA assay with active SaCas9:gRNA and an ACGGGT PAM sequence, which was validated with gel electrophoresis (FIG. 62F,G and FIG. 69F).

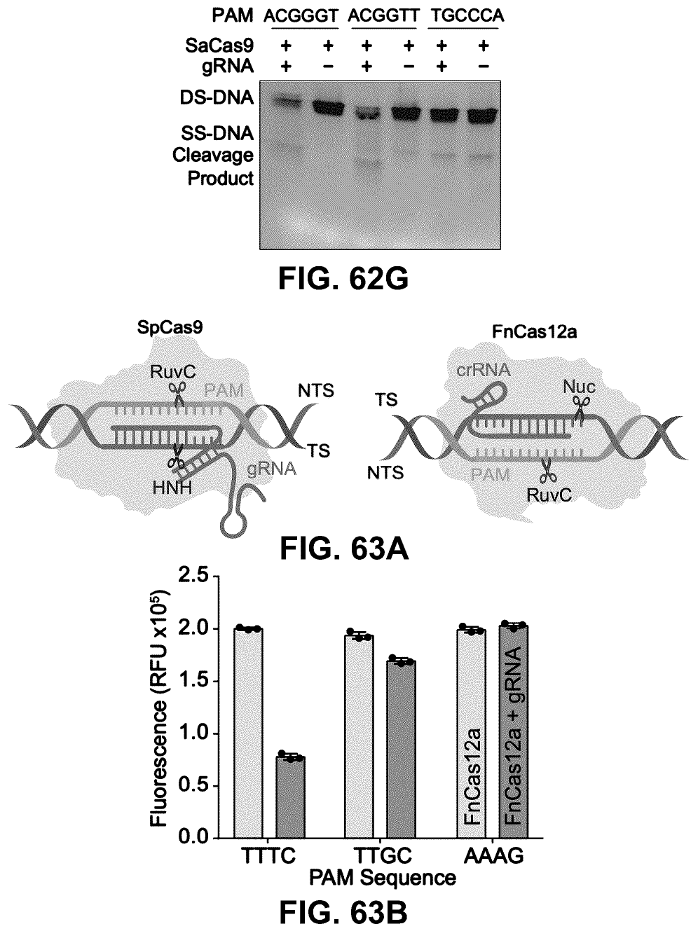

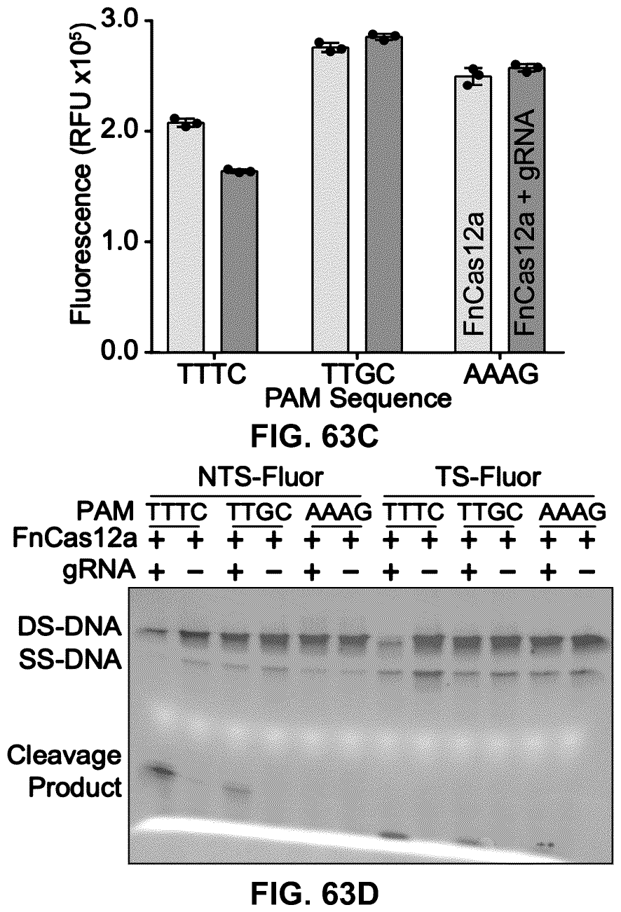

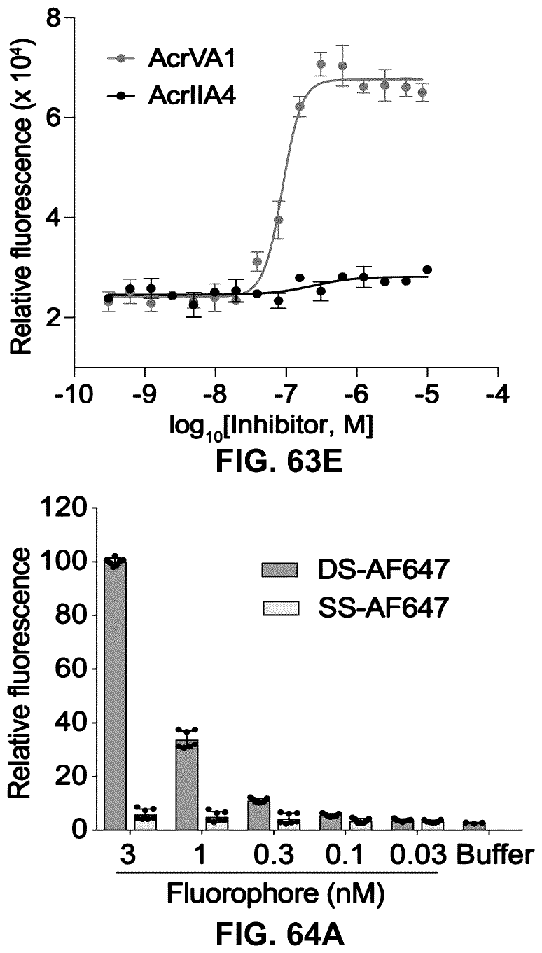

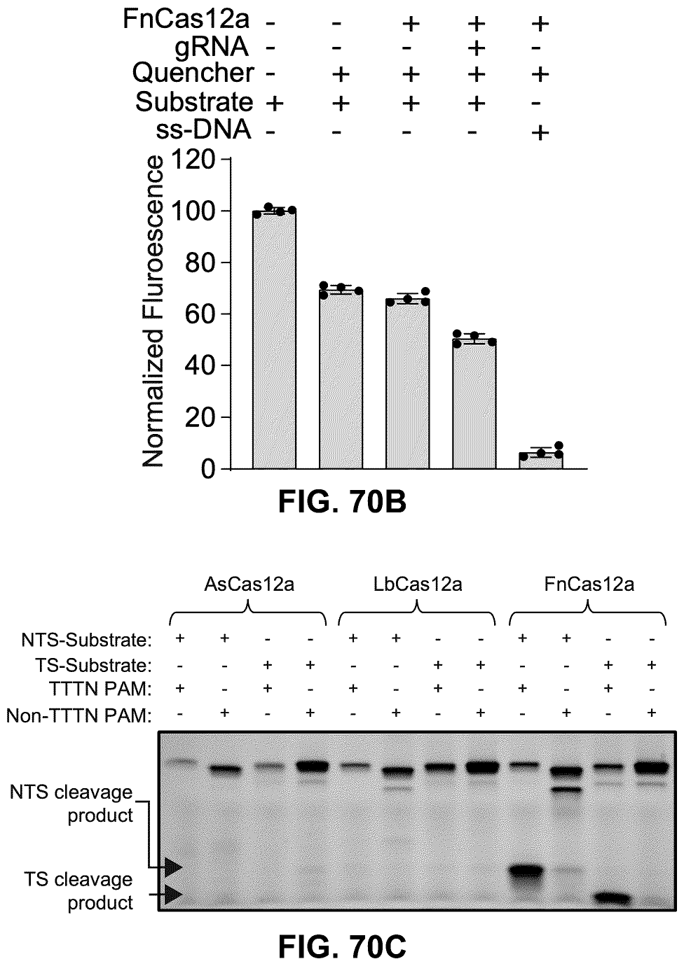

Next, Applicants adapted the assay for other Cas-family enzymes, starting with Cas12a. There are several mechanistic differences between the Cas9 and Cas12a families, including the number of nuclease domains (Cas9 has two; Cas12a has one), orientation of substrate binding (Cas9 recognizes a 3′-PAM; Cas12a recognizes a 5′-PAM), and additional enzymatic functionalities (Cas12a undergo non-specific collateral DNase and RNase activity, FIG. 63A).^{13, 28-30} To address these differences, Applicants prepared Cas12a substrates containing a 3′-fluorophore on either the non-targeting (NTS-Fluor) or targeting strand (TS-Fluor) (FIG. 63A). NTS-Fluor substrate showed higher PAM-dependent quenching than the TS-Fluor substrate (FIG. 63B,C and FIG. 70A,B), with the PAM-dependent cleavage observed in the CAA mirroring the gel electrophoresis results (FIG. 63D and FIG. 70C). The CAA was able to report on concentration-dependent inhibition by AcrVA1, but not AcrIIA4 (FIG. 63E), as reported previously.³¹ Overall, these studies indicate the relative ease of generalizing CAA for different and emerging CRISPR-associated nucleases.

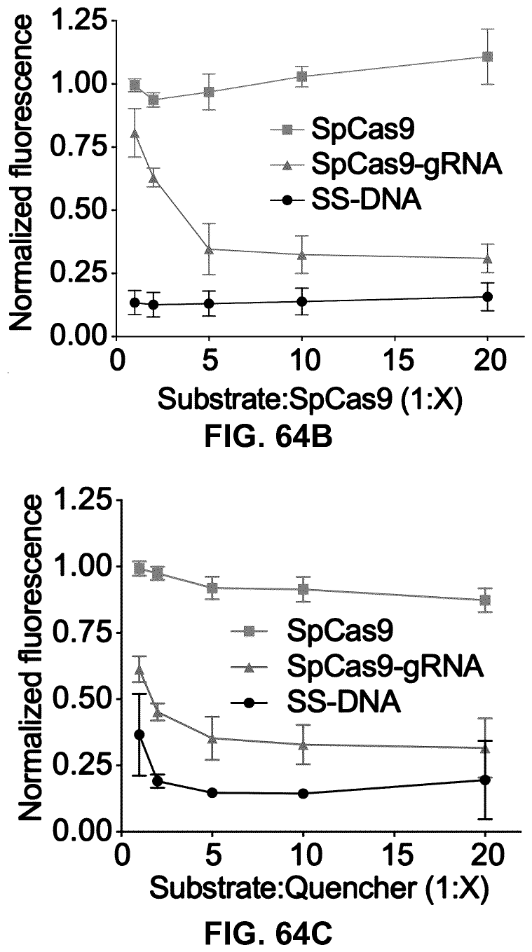

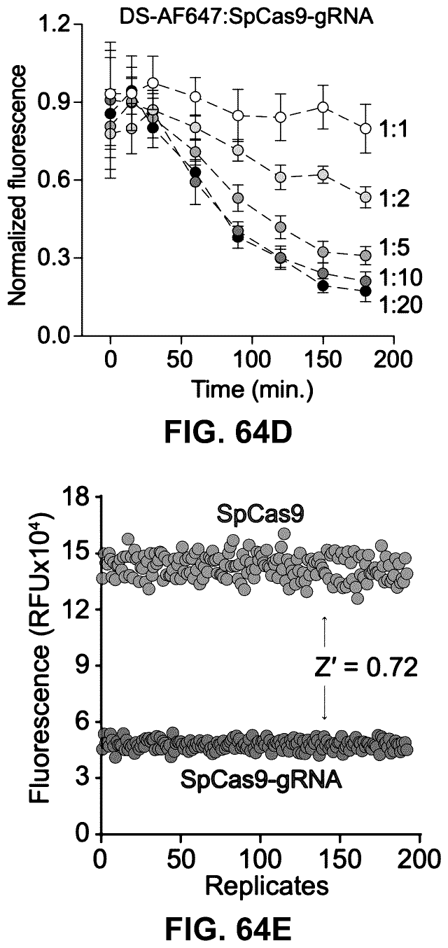

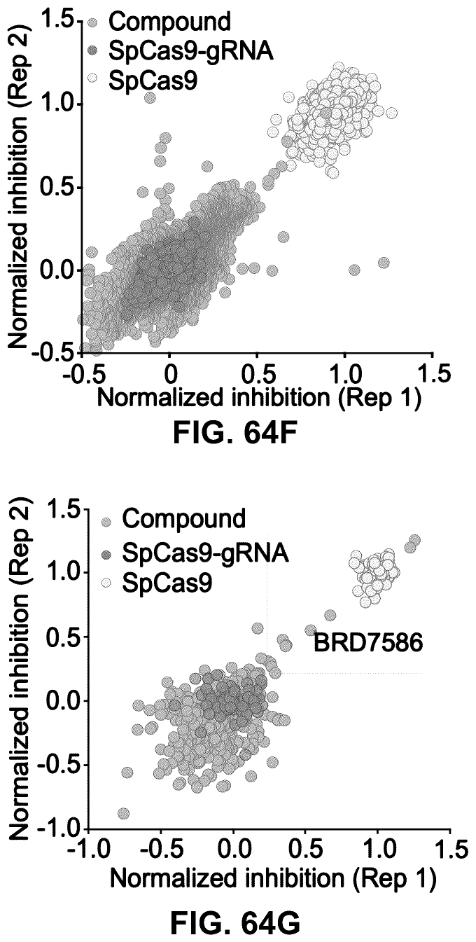

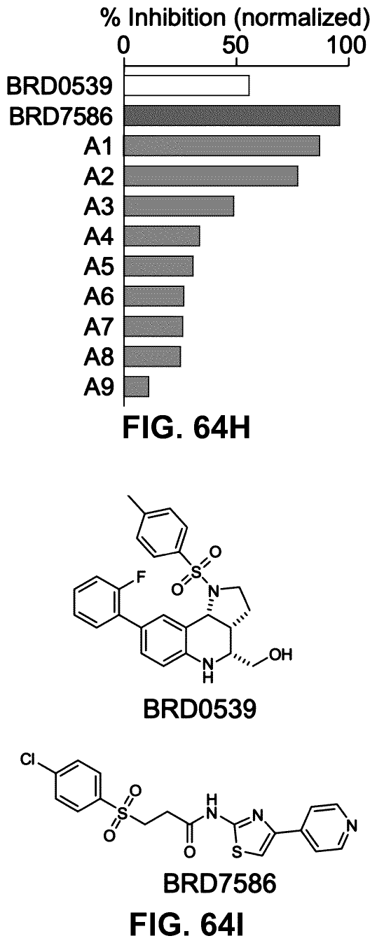

Optimization of CAA for high-throughput screening. To apply the CAA for high-throughput screening, it would need to sensitively detect SpCas9 activity within a reasonable time window. To minimize the interference from compound autofluorescence, Applicants used a red-shifted AlexaFluor 647-labeled DS-Fluor substrate (DS-AF647) for assay development. DS-AF647 was readily detectable down to 1 nM and could be efficiently quenched by the complementary strand bearing the quencher (Disp-Q, FIG. 64A). Applicants separately optimized the ratio of Disp-Q to 1 nM of DS-AF647 and SpCas9:gRNA to 1 nM of DS-AF647 and found that a 5-fold excess of each reagent relative to DS-AF647 yielded maximum quenching (FIG. 64B,C). Monitoring a time course of fluorescence quenching at various SpCas9:gRNA and DS-AF647 ratios showed that the reaction was effectively completed after 2.5 h (FIG. 64D). After adapting the assay for liquid handling systems to enable high-throughput screening, Applicants were able to detect 5 nM of SpCas9 using 0.5 nM of DS-AF647 with a Z′ factor (reports on the degree of separation between positive and negative control³²) of 0.72 (FIG. 64E).

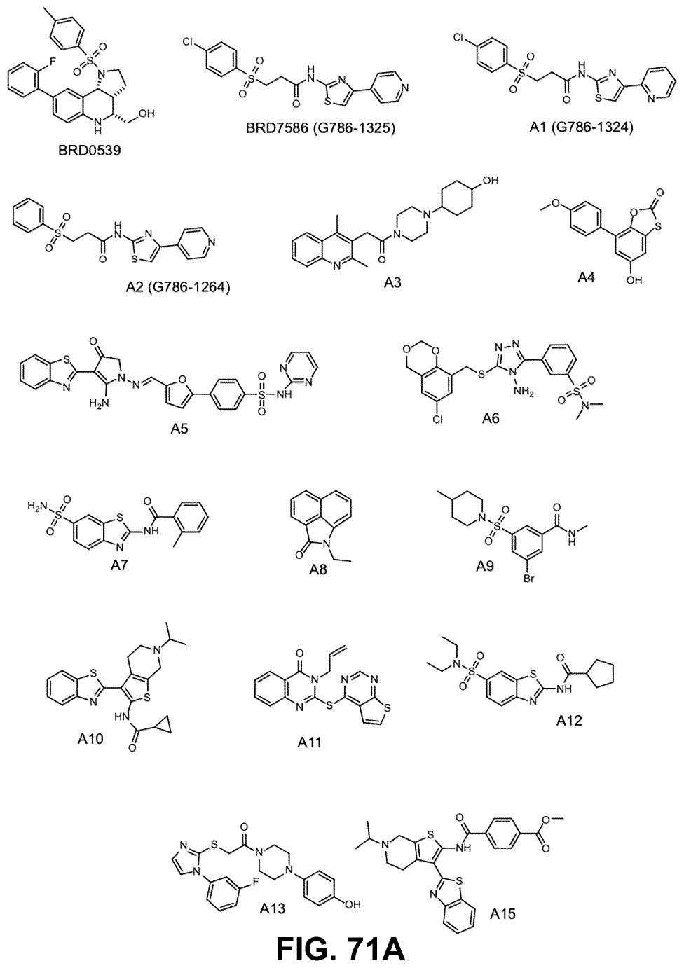

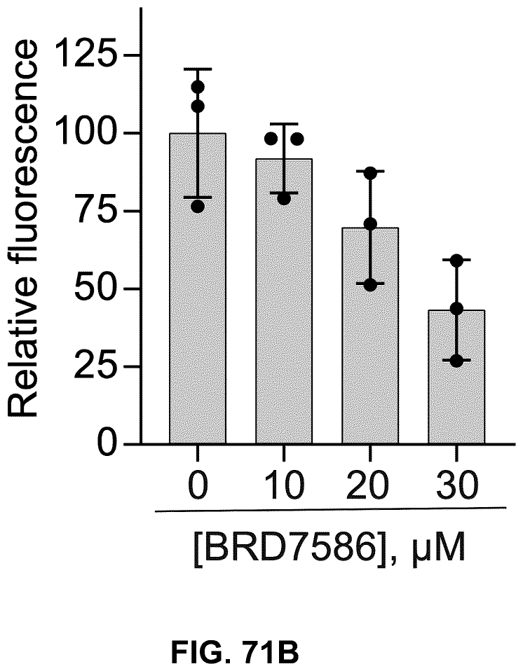

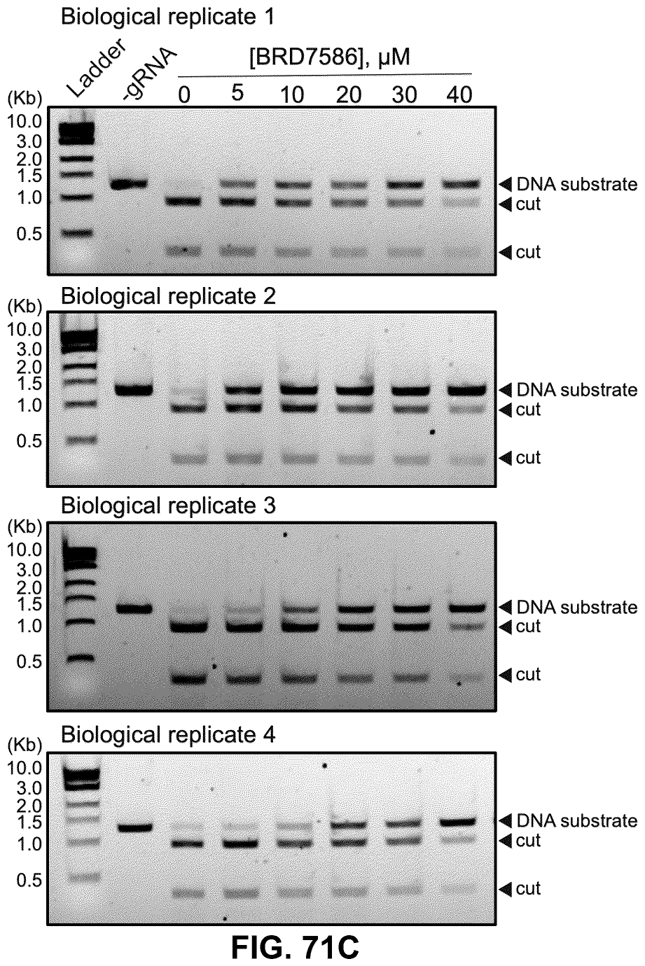

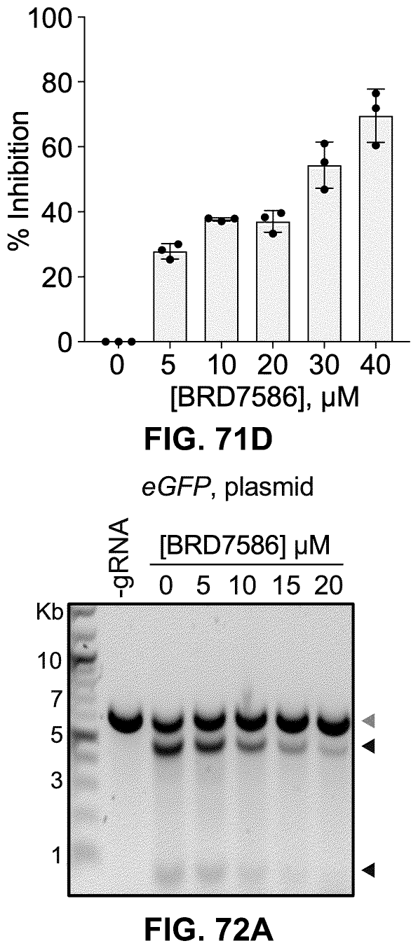

Primary and secondary screening. The primary screen assayed a selection of unique chemical scaffolds derived from commercially available compounds and known bioactive molecular libraries (Table 7). Auto-fluorescent compounds were removed by a counter screen. Overall, the CAA was used to assay 122,409 small molecules with over 2,500 unique chemical scaffolds (FIG. 64F, Table 7). Of these compounds, 547 were selected as hits (Z score >3 in both replicates) for testing in orthogonal cell-based secondary assays. Hits were tested in duplicate in an eGFP disruption assay, wherein U2OS.eGFP.PEST cells³³ transfected with SpCas9 plasmid and eGFP-targeting gRNA plasmid were incubated with 20 μM of the compounds. In this assay, any compound that inhibited SpCas9 would rescue the loss of eGFP fluorescence. Here, toxic and auto-fluorescent compounds with a high GFP signal in cells were removed as false positives. Of the 547 compounds tested in cellular assays, 15 compounds displayed a Z score >2 and 11 compounds displayed a Z score >3 in two independent screens (FIG. 64G). Next, Applicants tested the 15 compounds in a luminescence-based gain-of-signal HiBiT knock-in assay, which involves SpCas9-mediated homology-directed tagging of GAPDH with a short peptide that luminesces upon complementation with a subunit derived from nanoluciferase.³⁴ Complementarily, the eGFP-disruption assay is fluorescence-based and involves error-prone DNA repair. As a counter-screen to remove false positives caused by cell death (viability <80%), the cell viability was measured after incubation. Interestingly, the top three-performing compounds had a similar core structure, so Applicants selected BRD7586 for further studies. Furthermore, BRD7586 exhibited higher potency than BRD0539 in the HiBiT knock-in assay (FIG. 64H,I and FIG. 71A), and Applicants confirmed dose-dependent inhibition of SpCas9 by BRD7586 in CAA (FIG. 71B) and in vitro DNA cleavage assay (FIG. 71C, D).

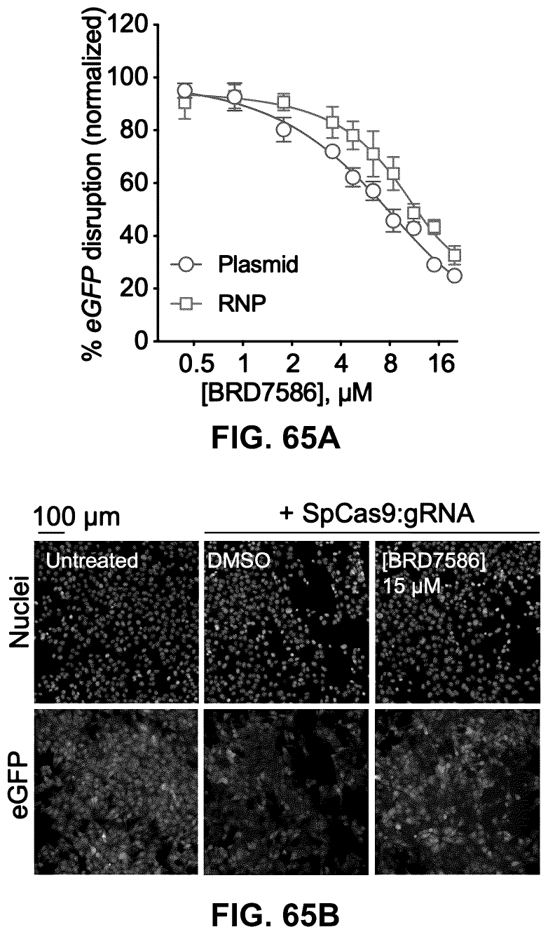

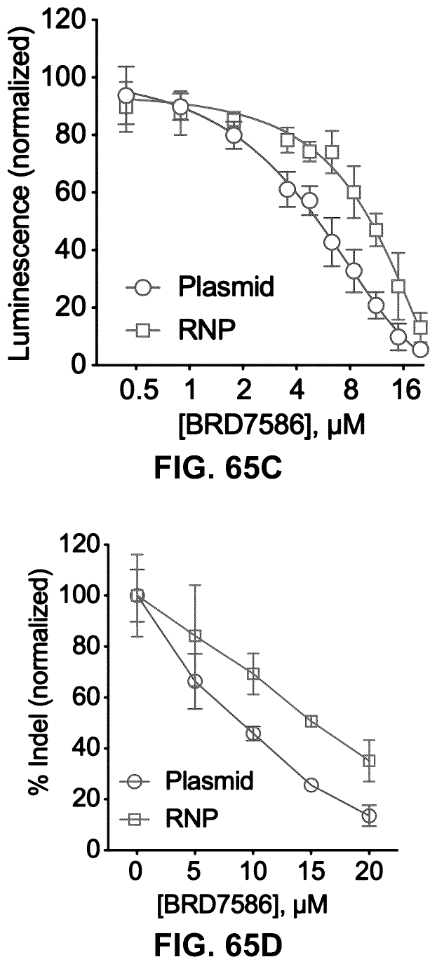

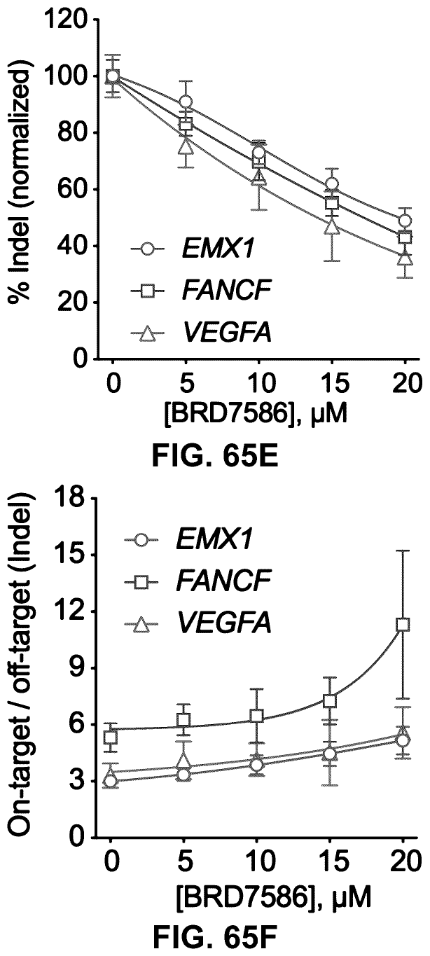

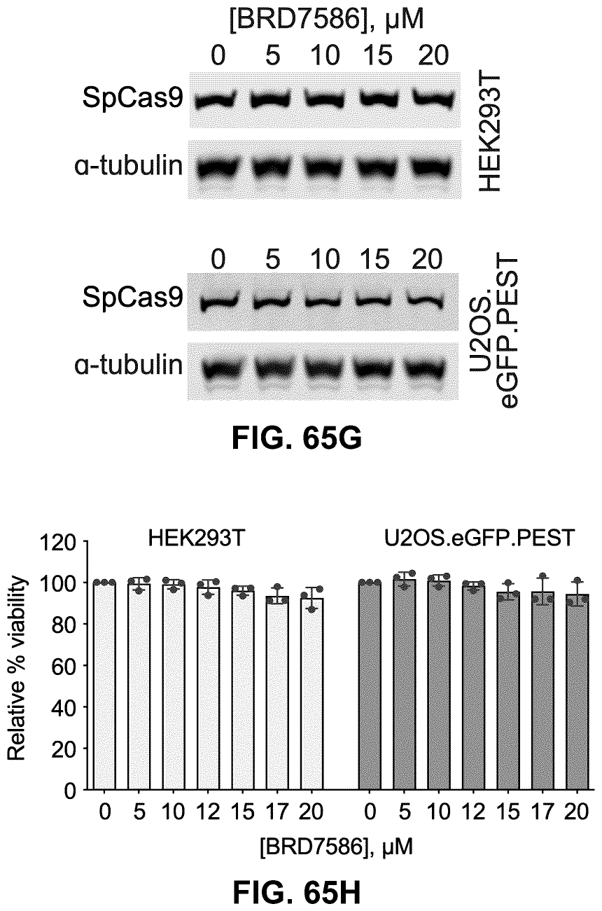

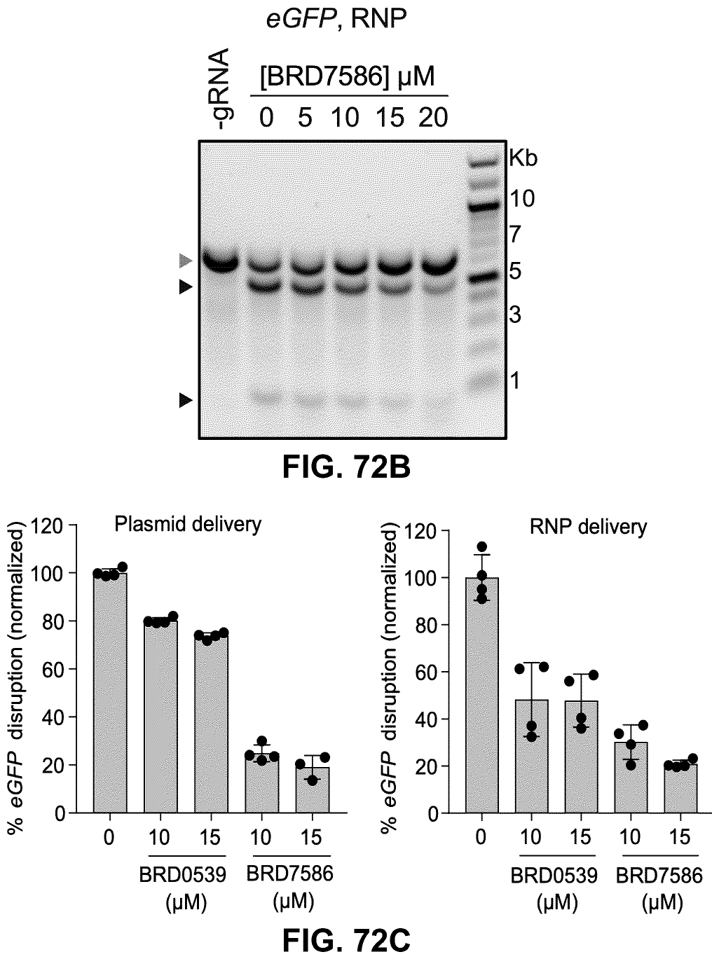

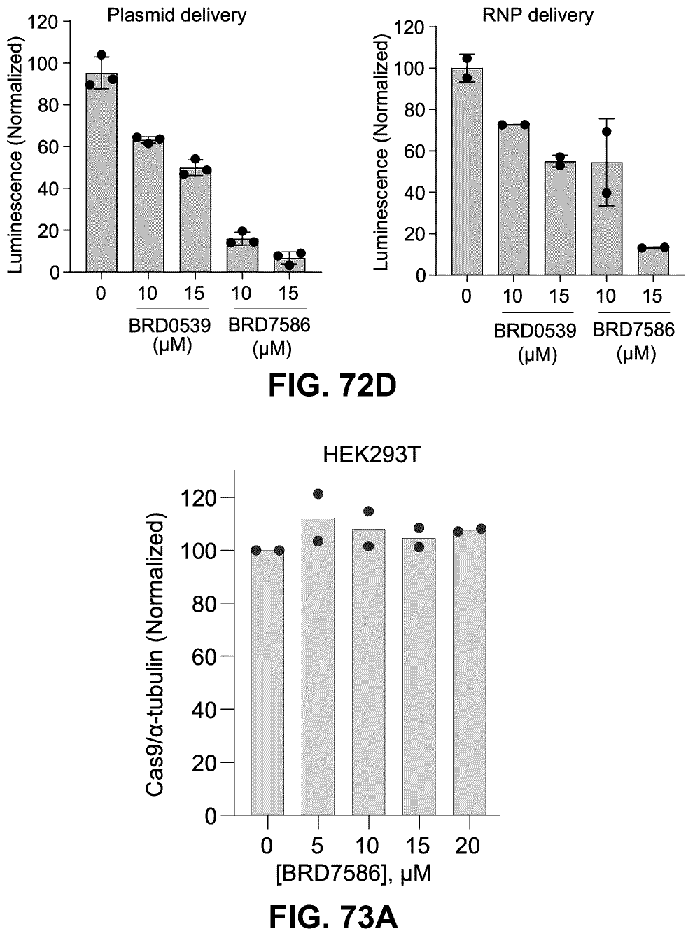

Cellular activities of BRD7586. Applicants next confirmed dose-dependent inhibition of SpCas9 by BRD7586 in multiple assays with an orthogonal readout (e.g., fluorescence, luminescence, and next-generation sequencing) and at multiple genomic loci. The EC₅₀ of BRD7586 in the eGFP disruption assay and HiBiT knock-in assay from three independent experiments were 6.2±1.2 μM and 5.7±0.36 μM, respectively, which are lower than the first-generation inhibitor BRD0539 at ˜12 μM (FIG. 65A-C). More importantly, BRD7586 inhibited the indel activity of SpCas9 at multiple genomic loci as determined by next-generation sequencing (FIG. 65D,E and FIG. 72A,B). Furthermore, BRD7586 enhanced the specificity of SpCas9. HEK293T cells were transfected with SpCas9 and gRNA plasmids targeting the gene EMX1, FANCF, or VEGFA and were incubated with BRD7586 for 48 h. Here, Applicants observed the enhanced on-target versus off-target ratio with an increasing amount of inhibitor (FIG. 65F). Moreover, BRD7586 exhibited substantially improved activity compared with first-generation BRD0539¹⁷ in the eGFP disruption and HiBiT knock-in assays (FIG. 72C,D).

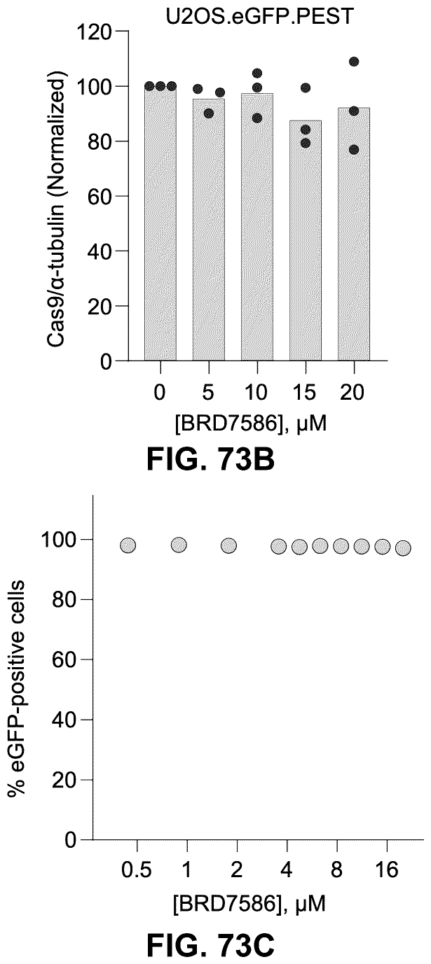

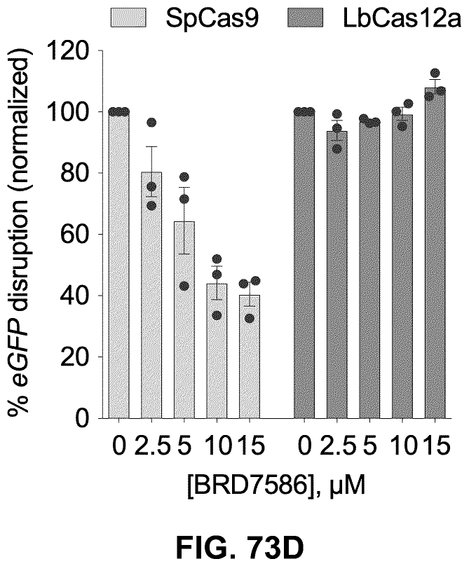

Applicants also note that BRD7586 inhibited SpCas9 in both HEK293T and U2OS.eGFP.PEST cells without altering its expression (FIG. 65G and FIG. 73A,B), without introducing cytotoxicity (FIG. 65H), and without affecting the eGFP expression in U2OS.eGFP.PEST cells in the absence of SpCas9:gRNA (FIG. 73C). While BRD7586 inhibited SpCas9, it did not affect the activity of structurally distinct LbCas12a, demonstrating its nuclease-selective activity (FIG. 73D). Finally, BRD7586 was 40% stable in mouse plasma.

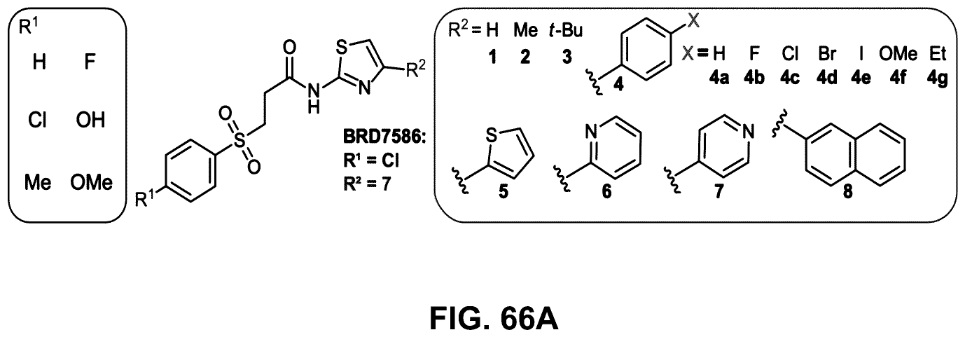

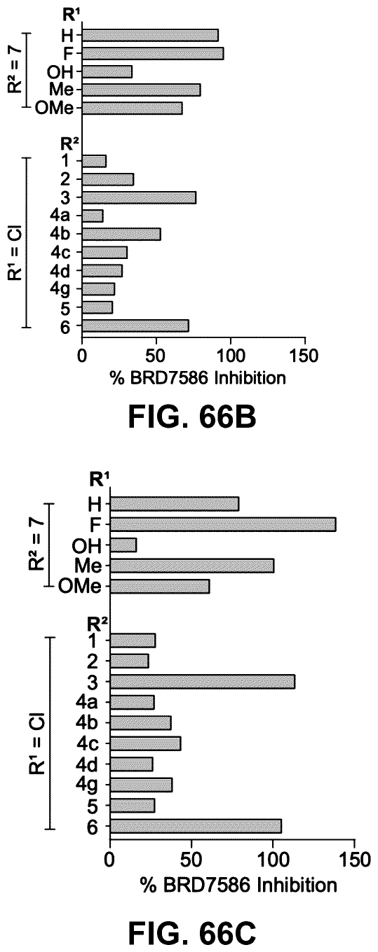

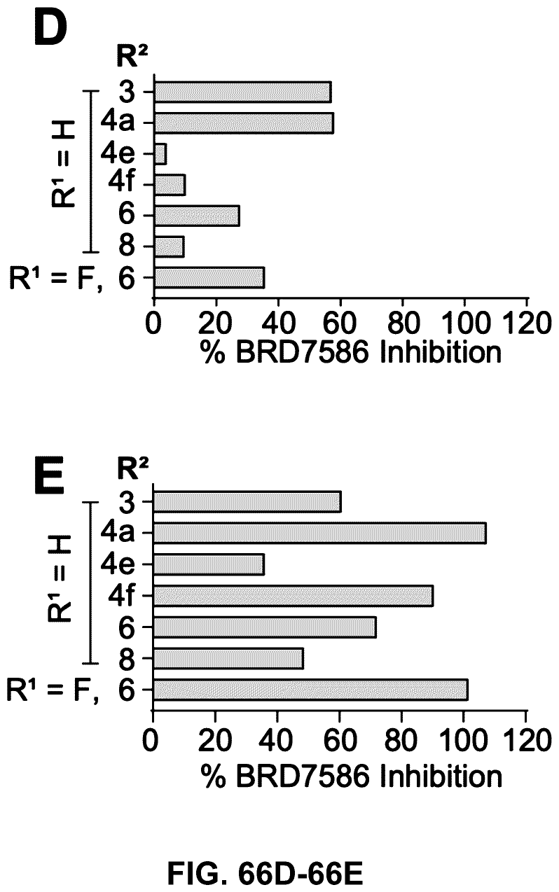

Structure-activity relationship studies of BRD7586. To identify the pharmacophore of the molecular scaffold, Applicants performed structure-activity relationship (SAR) studies against BRD7586. Applicants assembled analogs by individually substituting the R¹ and R² positions with different chemical functional groups (FIG. 66A) and tested these analogs in both eGFP disruption (FIG. 66B) and HiBiT knock-in assays (FIG. 66C). Keeping R¹ ═Cl and varying R² substantially changed the SpCas9 activity from the eGFP-disruption assay. First, replacing the pyridyl group (R²=6, 7) with phenyl rings (R²=4a, 4b, 4c, 4d, 4g), a 2-thienyl group (R²=5), or smaller substituents such as hydrogen (R²=1) or a methyl group (R²=2) greatly decreased the activity. Instead, replacing the pyridyl ring with a tert-butyl group (R²=3) only slightly decreased the activity. Keeping R²=7 and varying R¹ only slightly impacted the activity, with the exception of the hydroxyl substitution. These compounds were tested in the HiBiT knock-in assay by incubating 15 μM of each analog with HEK293T cells transfected with SpCas9:gRNA ribonucleoprotein (RNP) for 24 h (FIG. 66C). Similar trends were observed in this orthogonal assay, except that a higher apparent activity was observed in a few compounds that also exhibited slight toxicity in this assay.

In addition to single modifications, Applicants examined double modifications of BRD7586. These analogs were also tested in the eGFP disruption (FIG. 66D) and HiBiT knock-in (FIG. 66E) assays showing similar trends to the individual substitutions, indicating that the opposite handles of the compound act to stabilize the pocket independently. Once again, the HiBiT knock-in assay displayed higher inhibition than observed in the eGFP disruption assay, though Applicants observed some toxicity that resulted in a higher apparent activity.

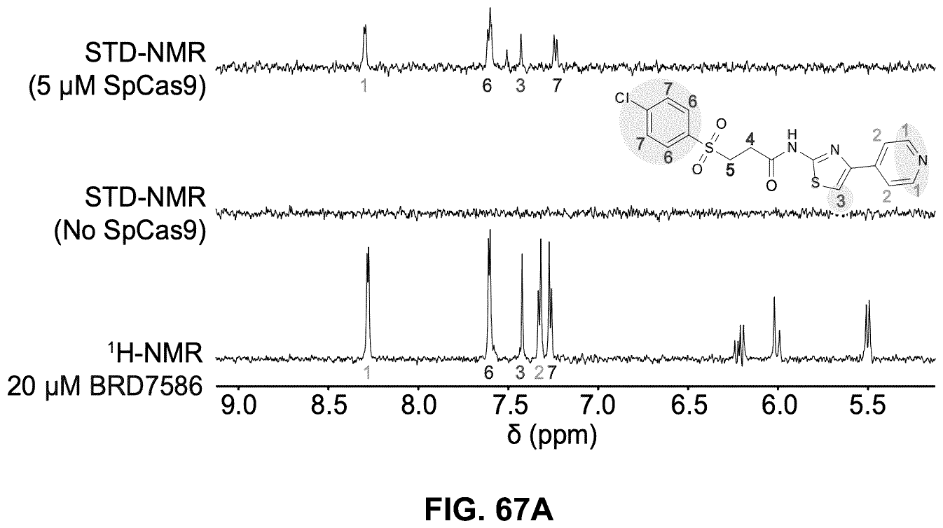

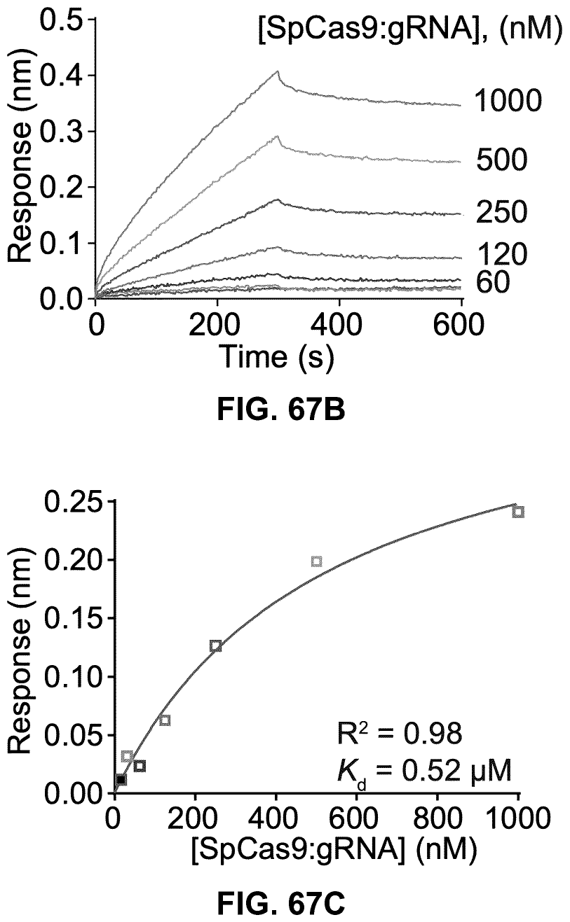

Biochemical activity of BRD7586. Similar to the cellular studies, Applicants characterized the activity and binding of BRD7586 to SpCas9 using orthogonal readouts (e.g., NMR, biolayer interferometry). Applicants used saturation transfer difference (STD) NMR to probe the binding of 20 μM of BRD7586 to 5 μM of SpCas9:gRNA complex. Applicants observed the STD NMR signal from marked protons, suggesting that these are directly involved in binding to the SpCas9 complex (FIG. 67A). The same STD NMR experiment in the absence of protein did not display the STD NMR signal, suggesting that the signal does not arise from aggregation or other artefacts. The STD NMR suggests an interaction between SpCas9 and the phenyl group protons, the thiazole proton, and the ones adjacent to the nitrogen on the pyridyl ring, but not with the other protons.

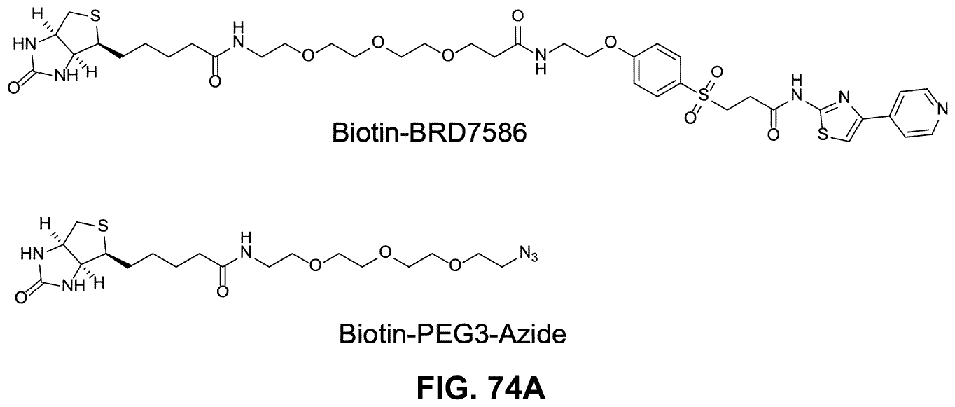

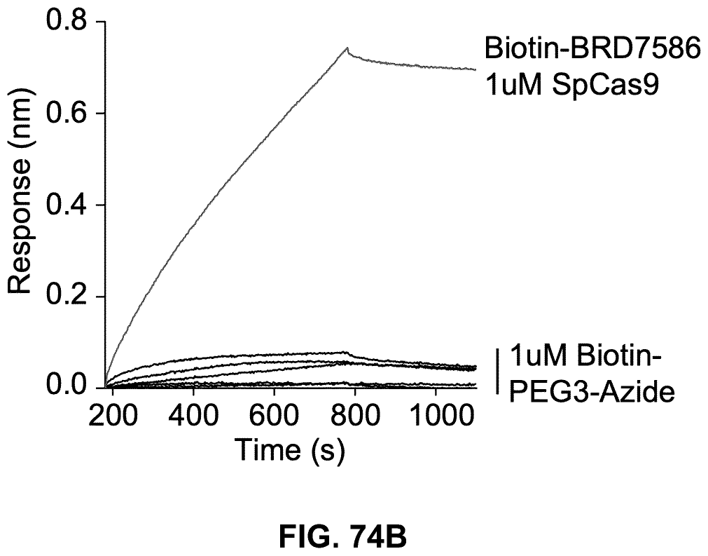

Based on the SAR and STD NMR studies, Applicants identified the para position of the phenyl ring as a likely tolerable linker attachment site on BRD7586 (FIG. 66). Additionally, the STD NMR and SAR studies indicated that the pyridine group was involved in the binding so would not tolerate any substitutions. Applicants attached a biotin-PEG3 to the para position of the phenyl ring to synthesize biotin-BRD7586 (FIG. 74A). Biolayer interferometry studies using 1 μM of biotin-BRD7586 with SpCas9:gRNA complex suggested a dissociation constant of 0.52 μM (FIG. 67B,C). No detectable binding was observed when the SpCas9:gRNA complex was added to biotin-PEG3-azide in the absence of the BRD7586 parent scaffold (FIG. 74B).

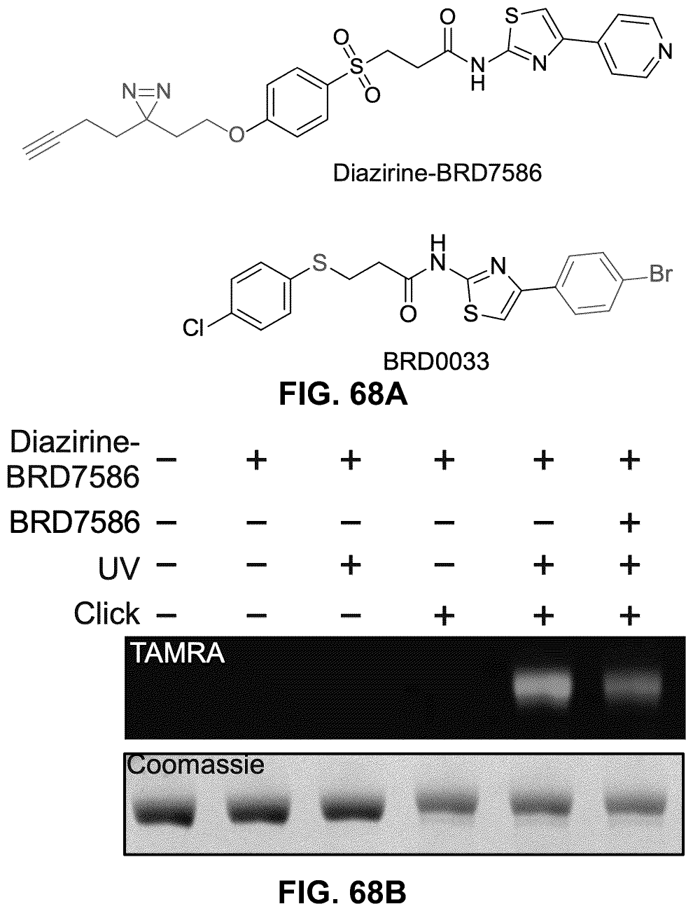

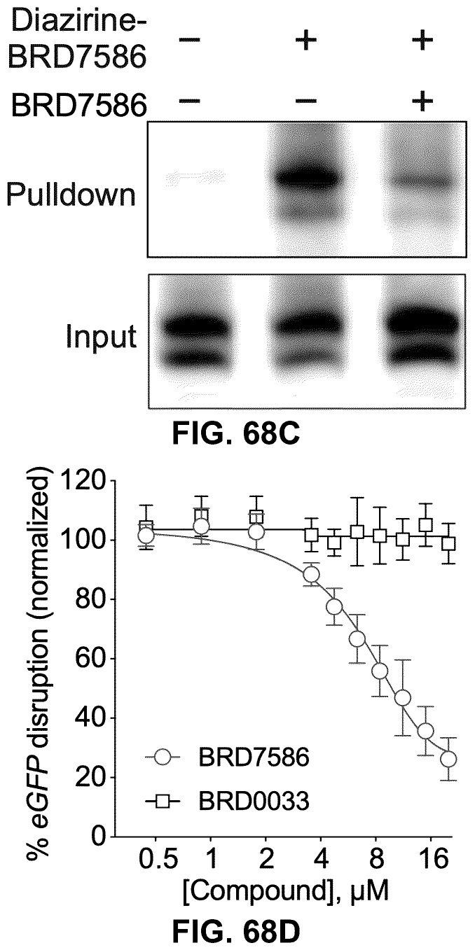

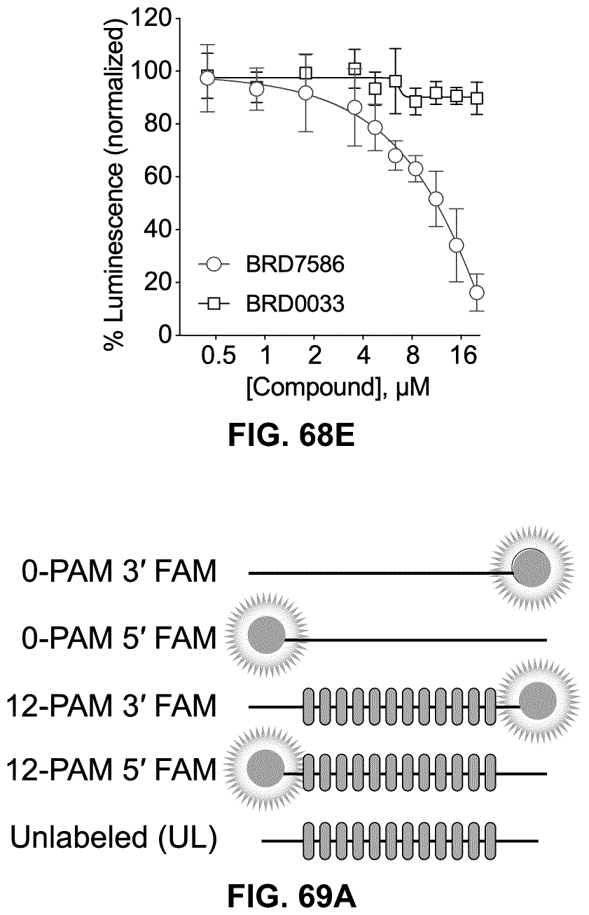

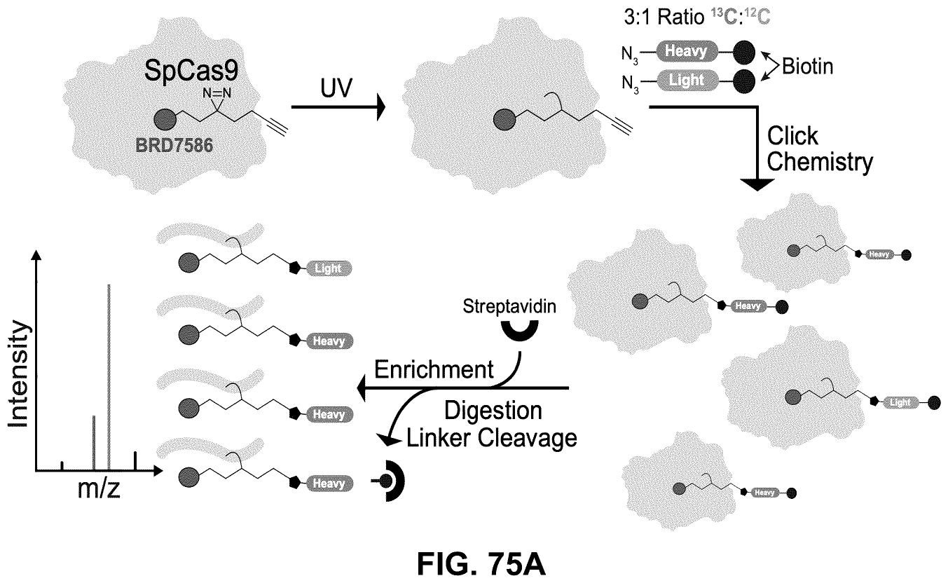

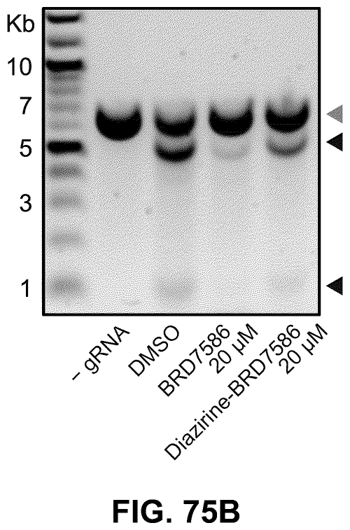

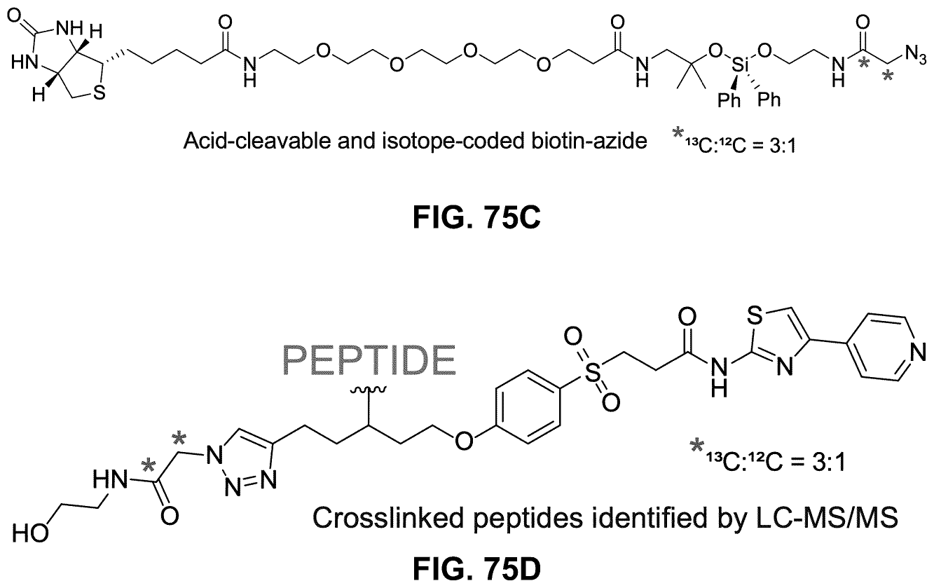

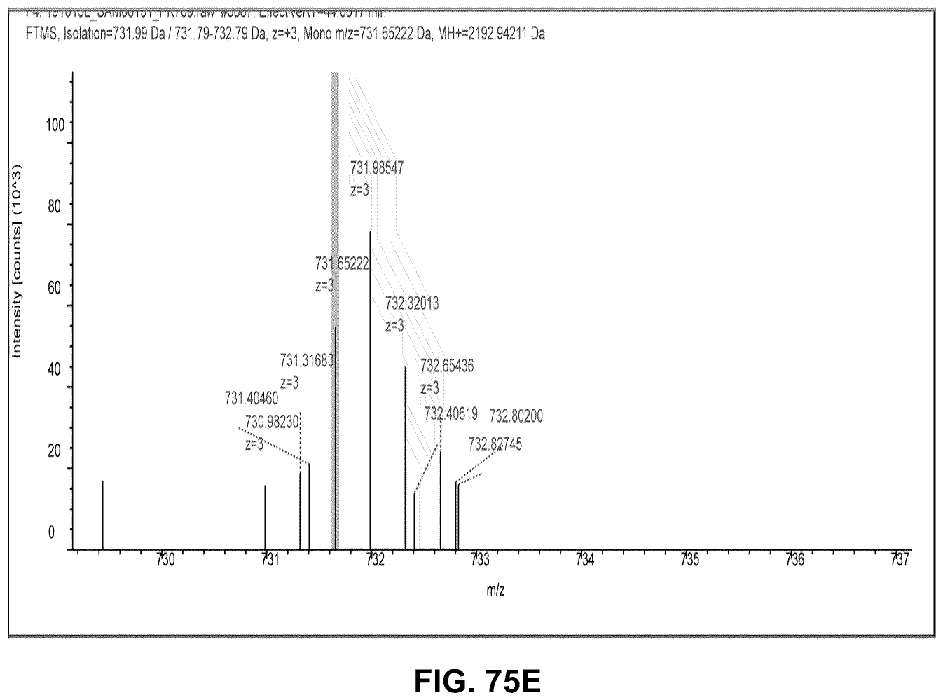

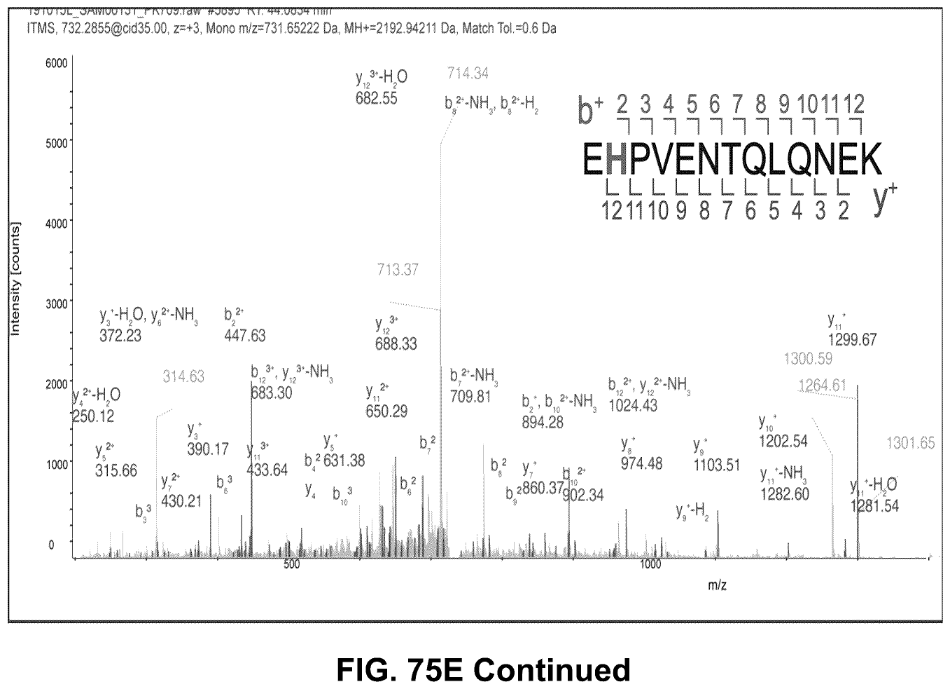

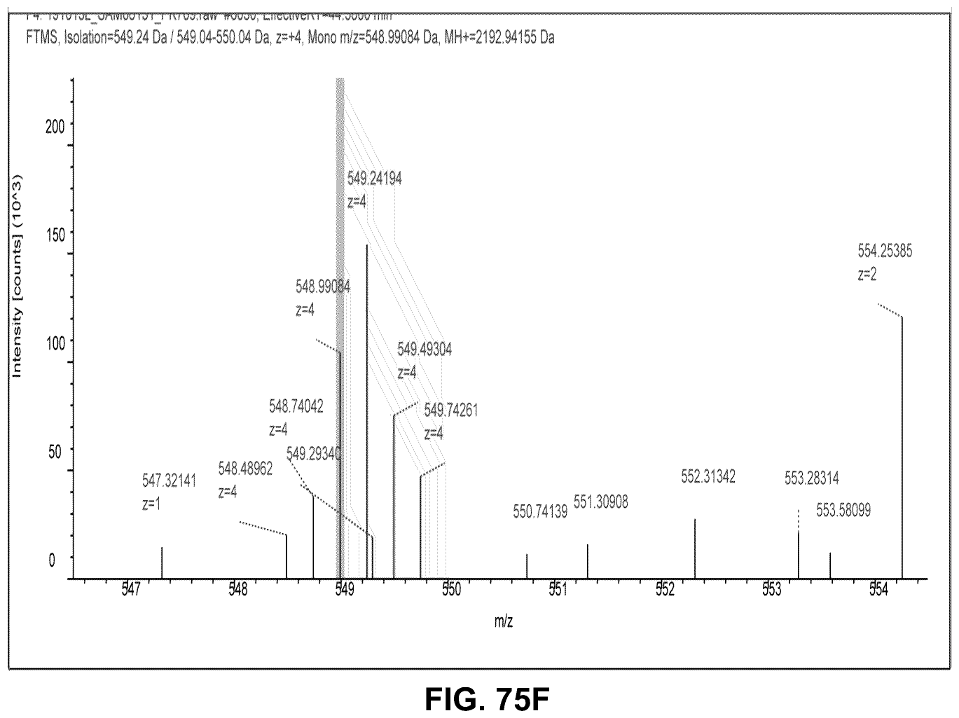

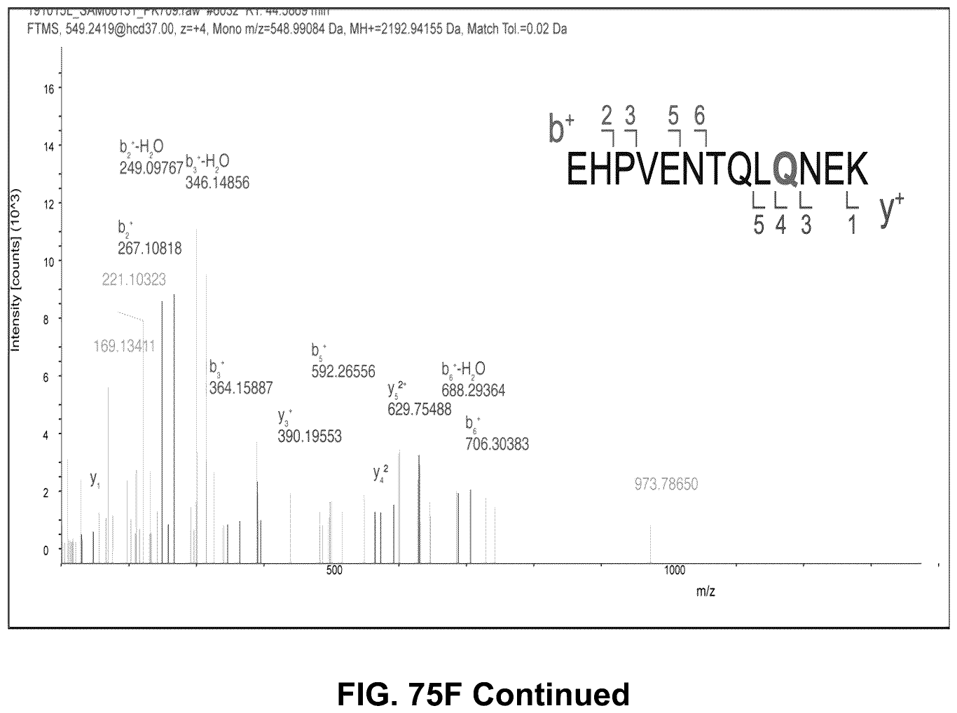

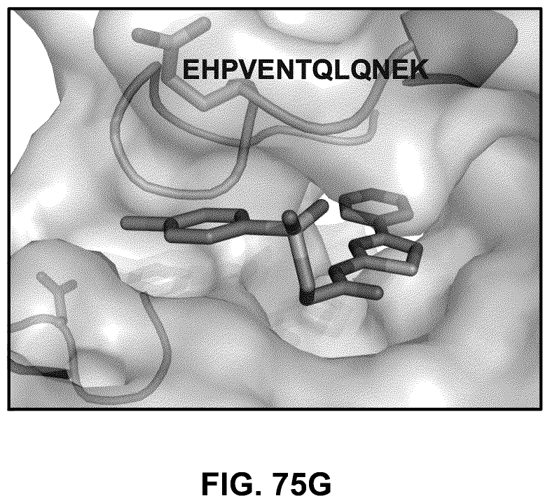

Target engagement and design of inactive analog. Applicants used a photoaffinity labeling strategy to demonstrate target engagement in cells via a diazirine-based BRD7586 (FIG. 68A).³⁵⁻³⁷ Based on the SAR studies, Applicants designed and synthesized a photo-crosslinking probe (diazirine-BRD7586) bearing a minimalist tag containing a photoreactive diazirine moiety and alkyne handle. The probe inhibited SpCas9 in cells, in accordance with the SAR results (FIG. 75A). To establish crosslinking of diazirine-BRD7586 to its target, SpCas9:gRNA complex was incubated with diazirine-BRD7586, and the mixture was photo-irradiated. Click chemistry with TAMRA-azide allowed the visualization of the crosslinking product through in-gel fluorescence analysis. Applicants demonstrated successful covalent conjugation of the probe to SpCas9. Furthermore, the crosslinking was selective, as demonstrated via competition with BRD7586 (FIG. 68B). Applicants then validated target engagement of BRD7586 via photo-irradiation in live cells treated with diazirine-BRD7586 in the presence and absence of BRD7586. After cell lysis and click chemistry with biotin-azide, the crosslinked proteins were enriched using streptavidin pull-down and immunoblotting to reveal the formation of crosslinks between diazirine-BRD7586 and SpCas9 (FIG. 75B). In the presence of BRD7586 as a competitor, this binding was abolished, demonstrating target engagement in live cells (FIG. 68C).

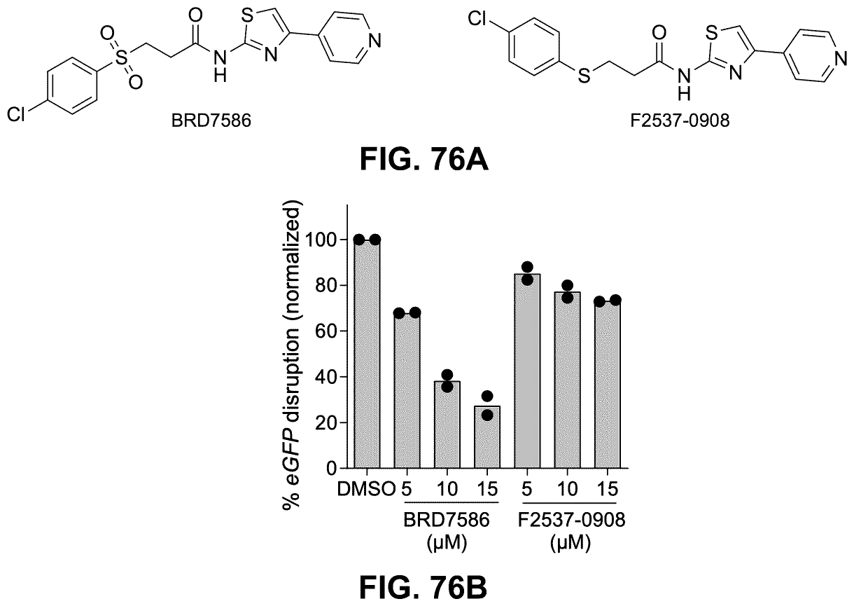

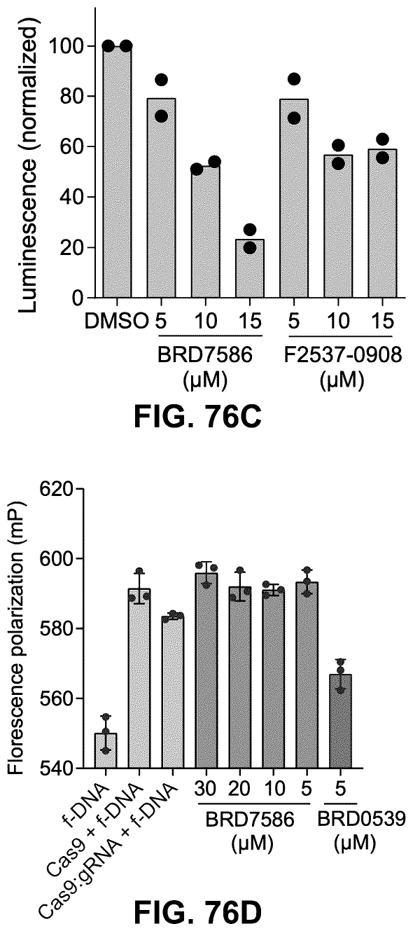

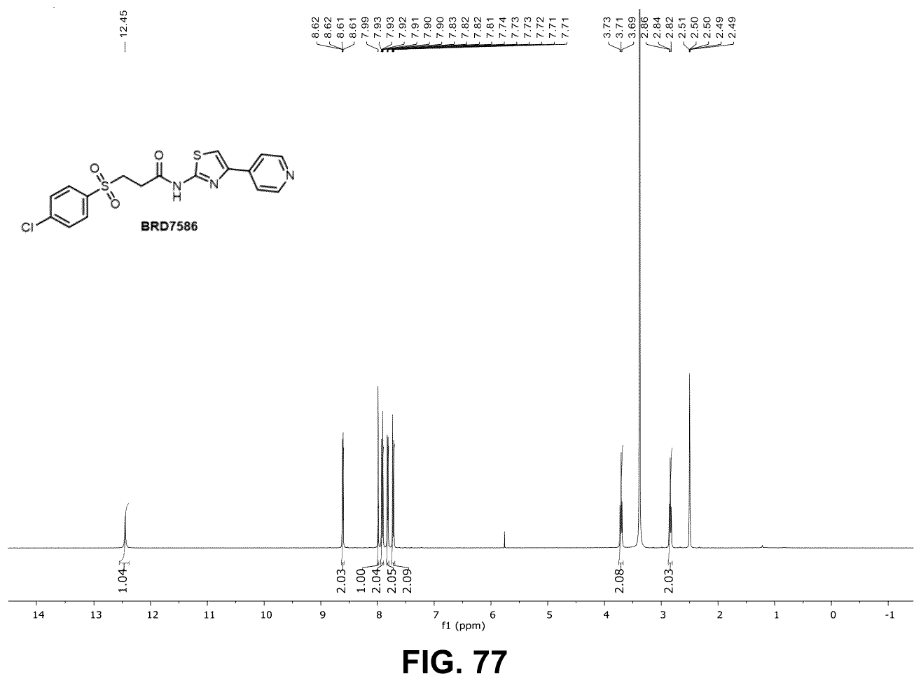

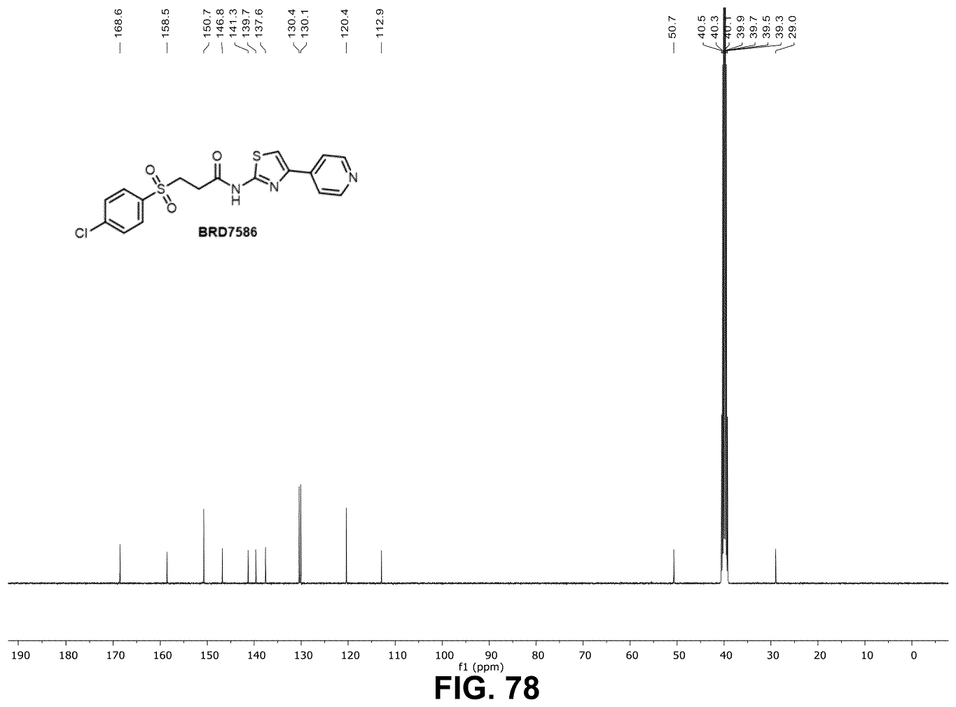

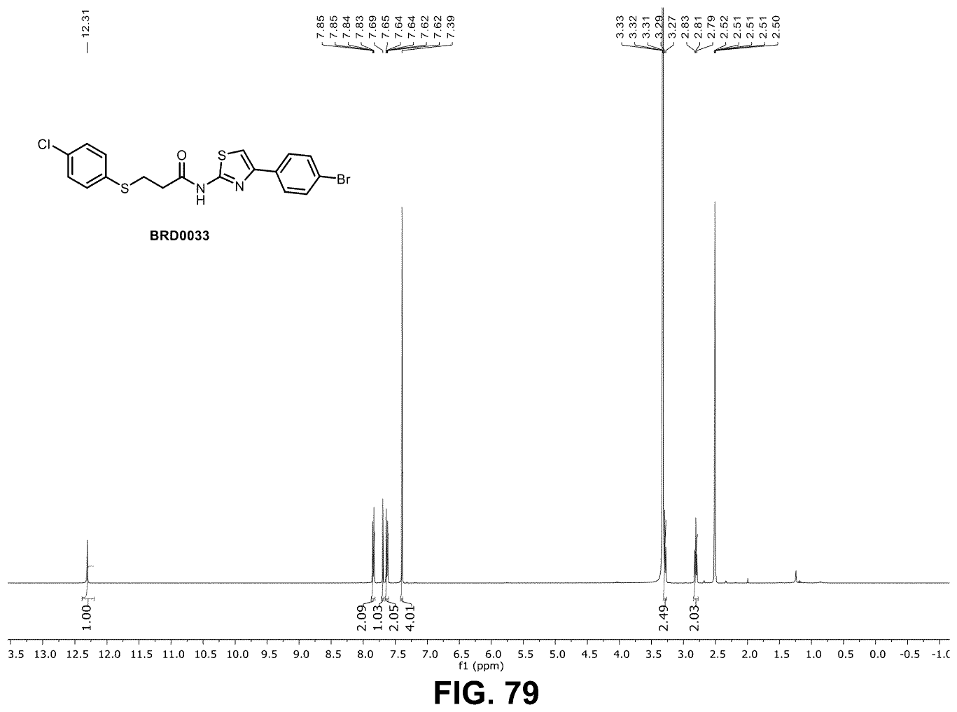

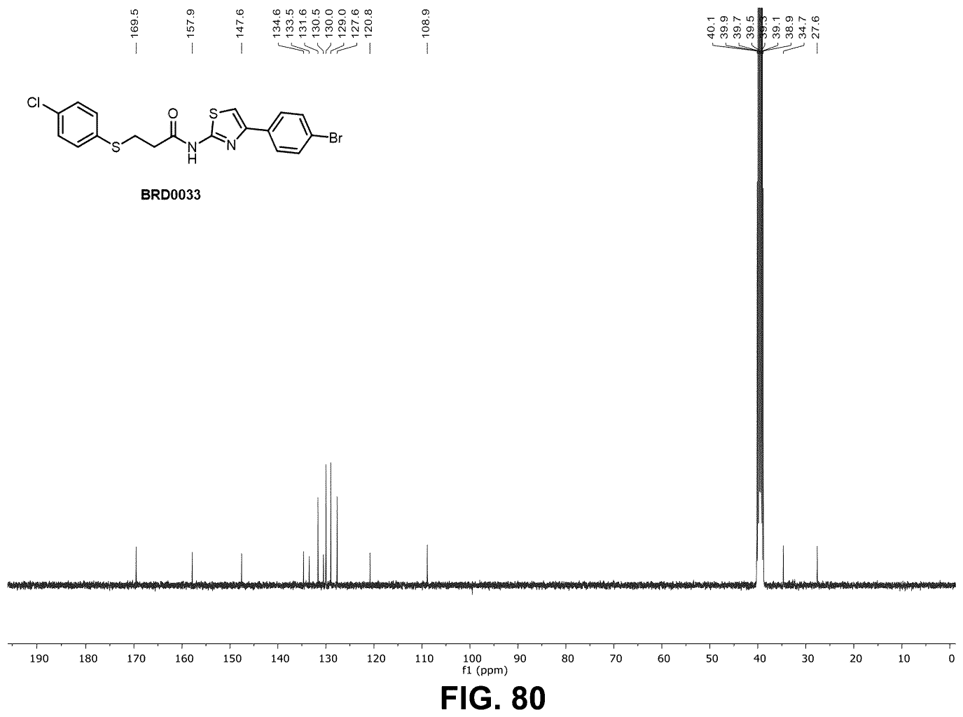

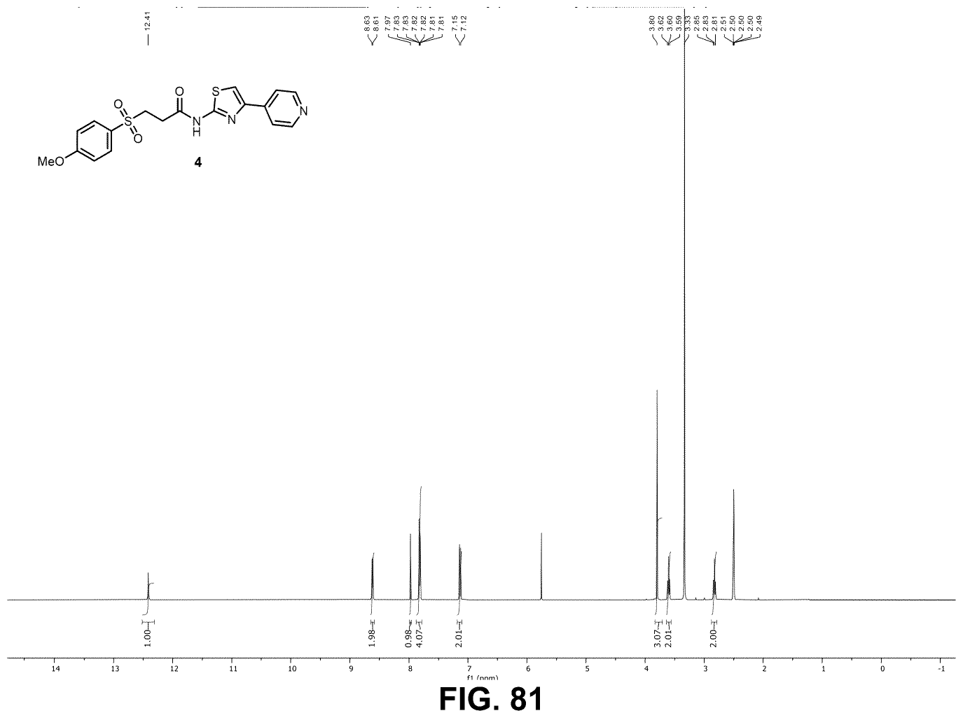

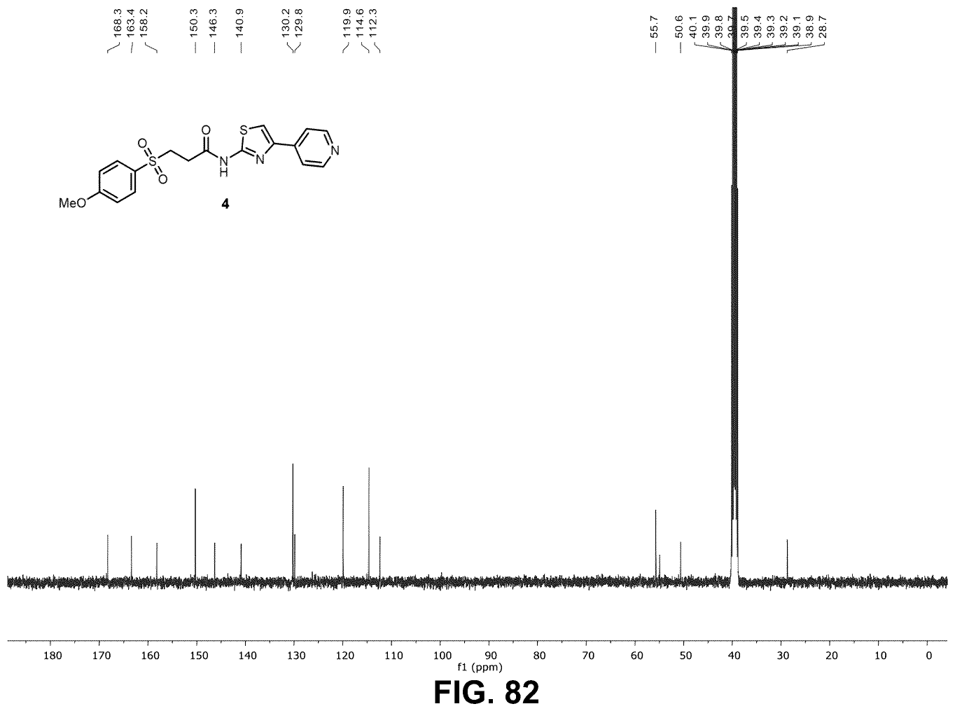

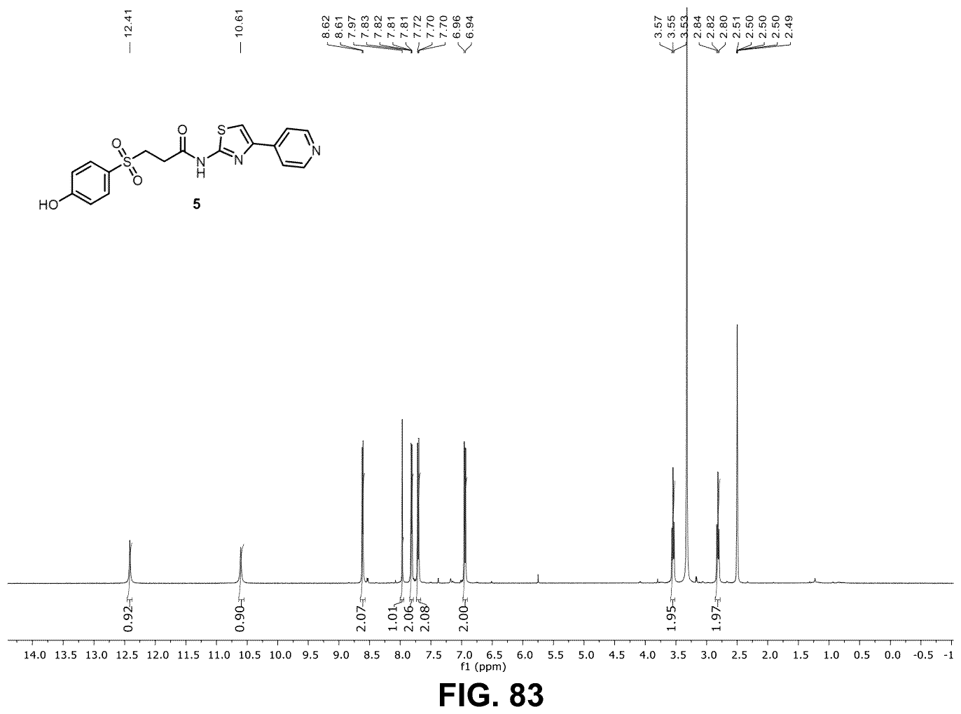

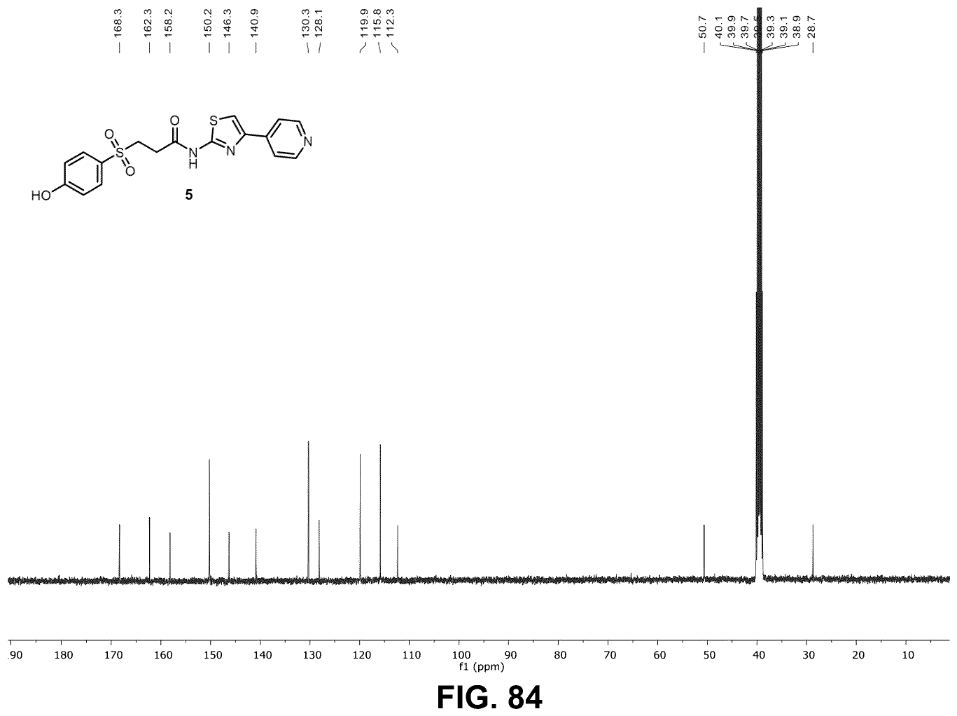

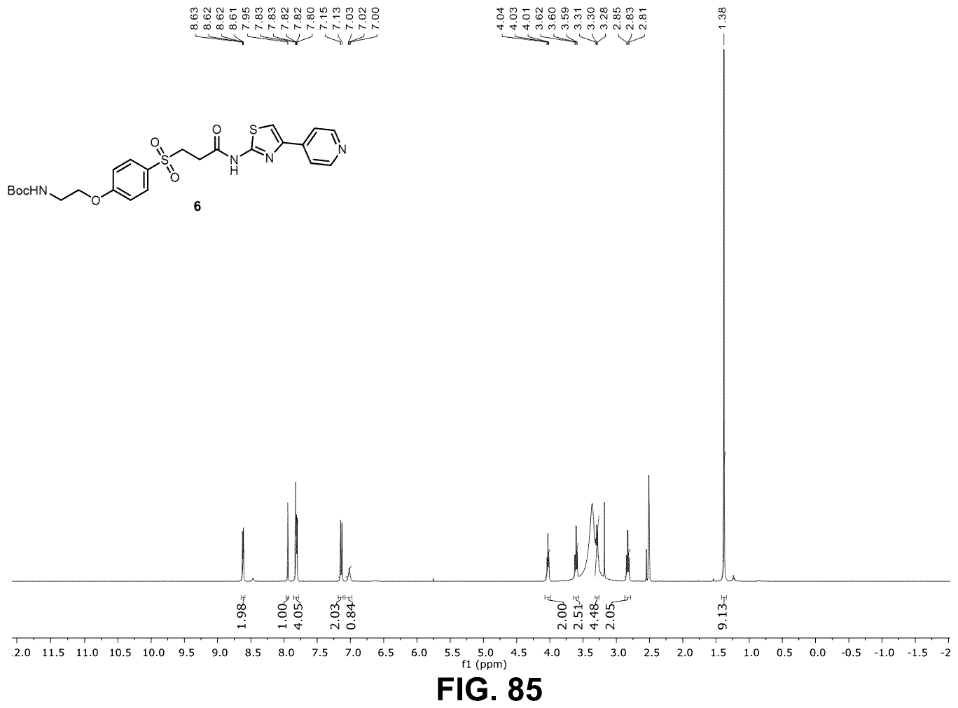

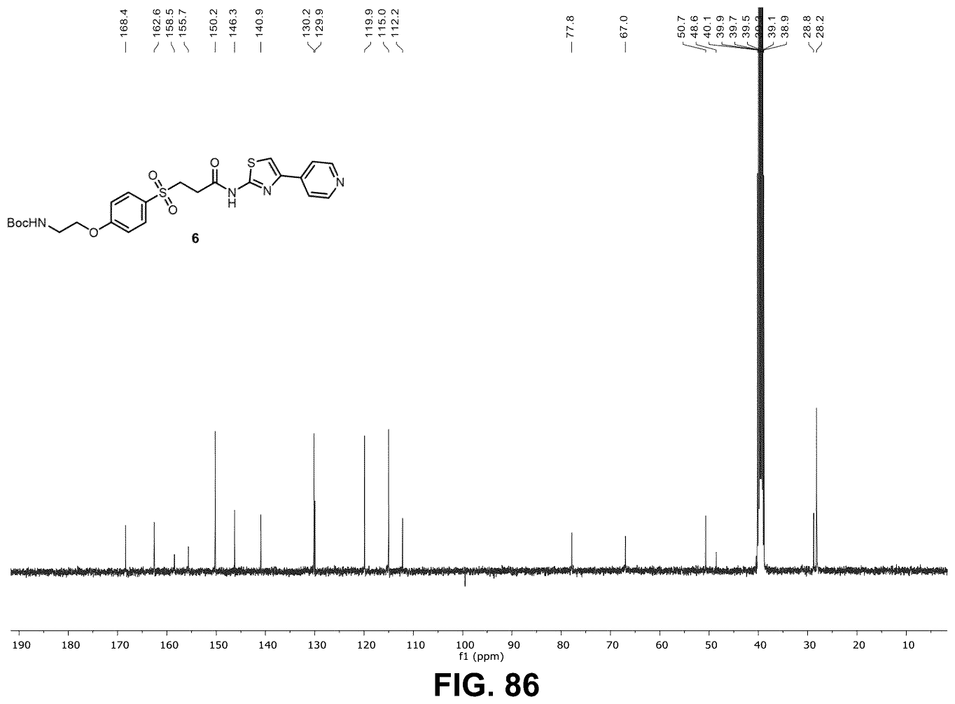

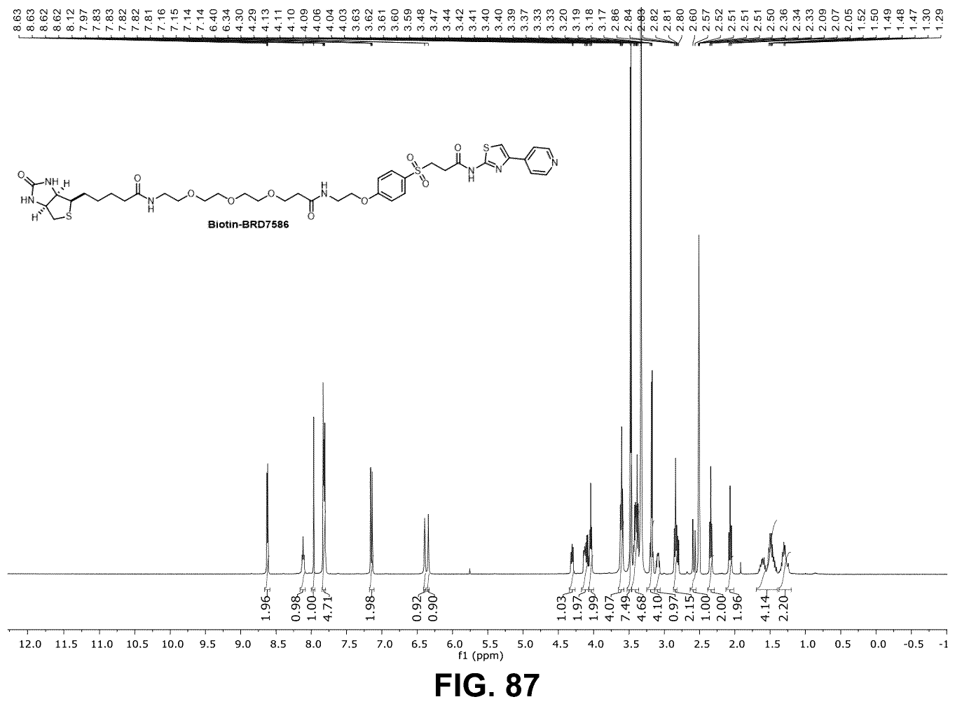

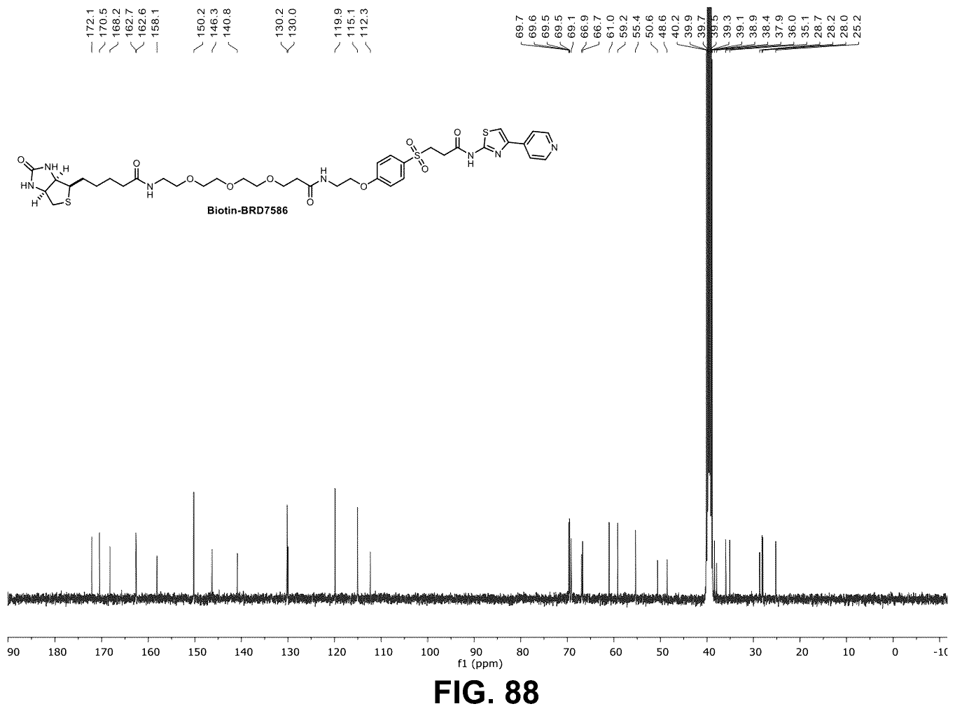

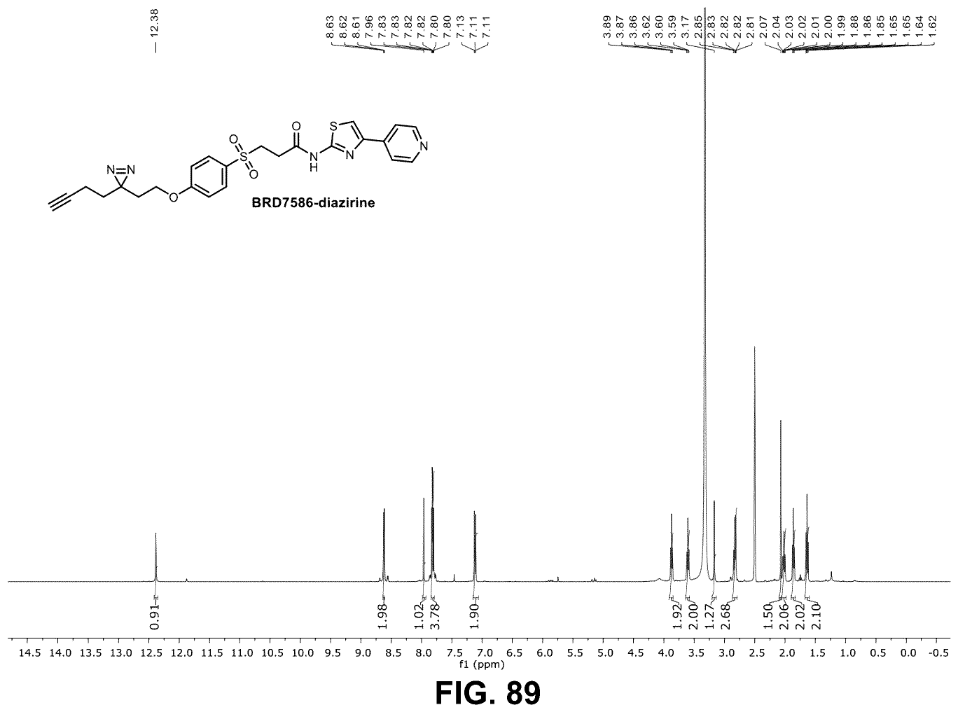

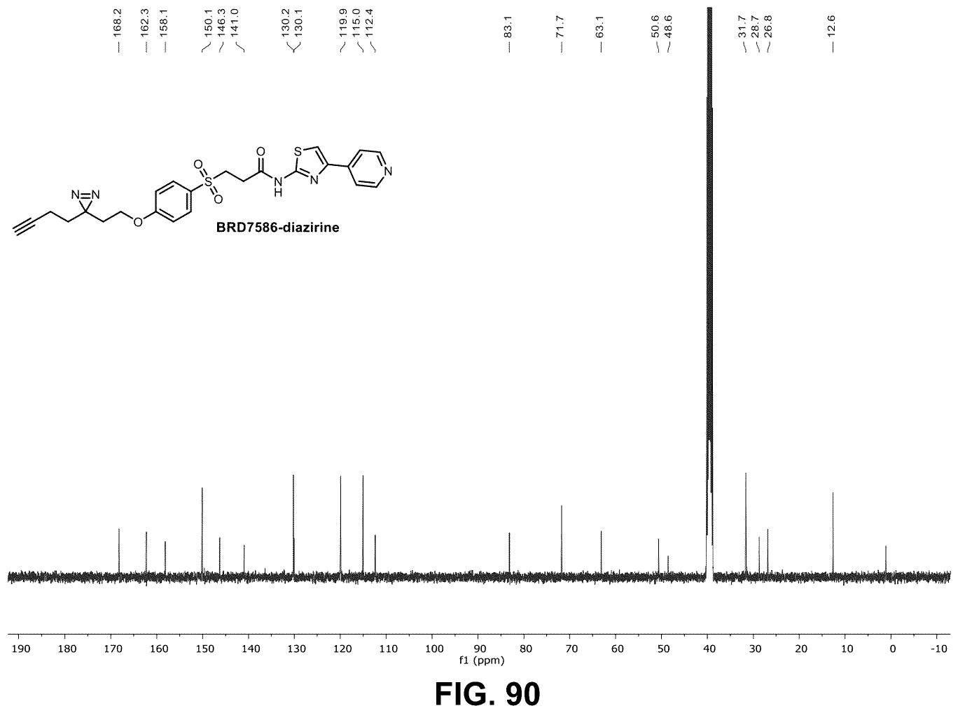

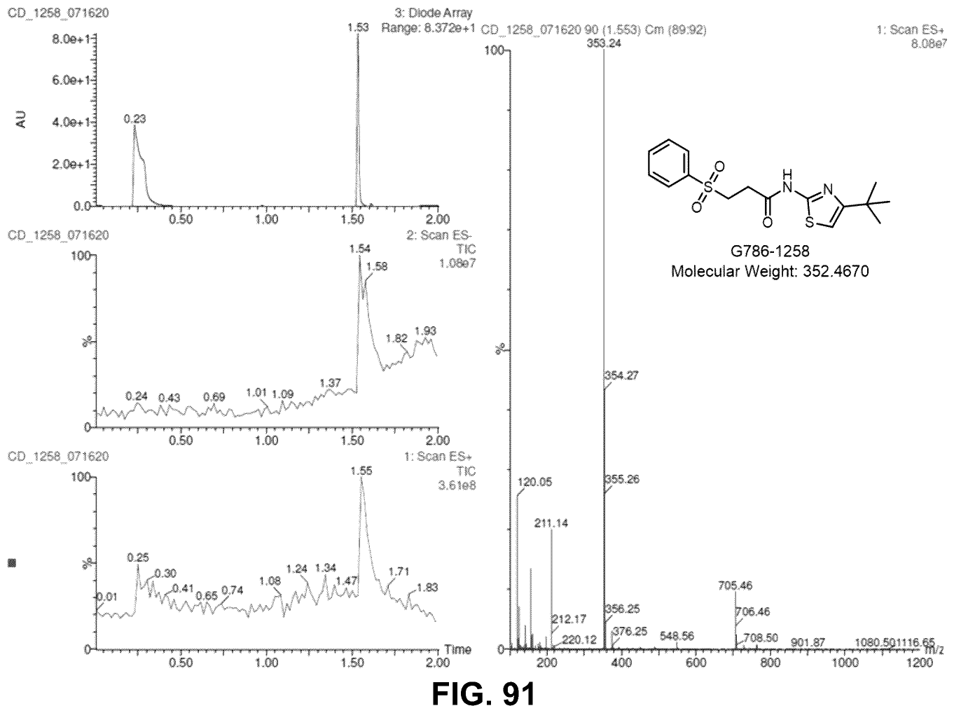

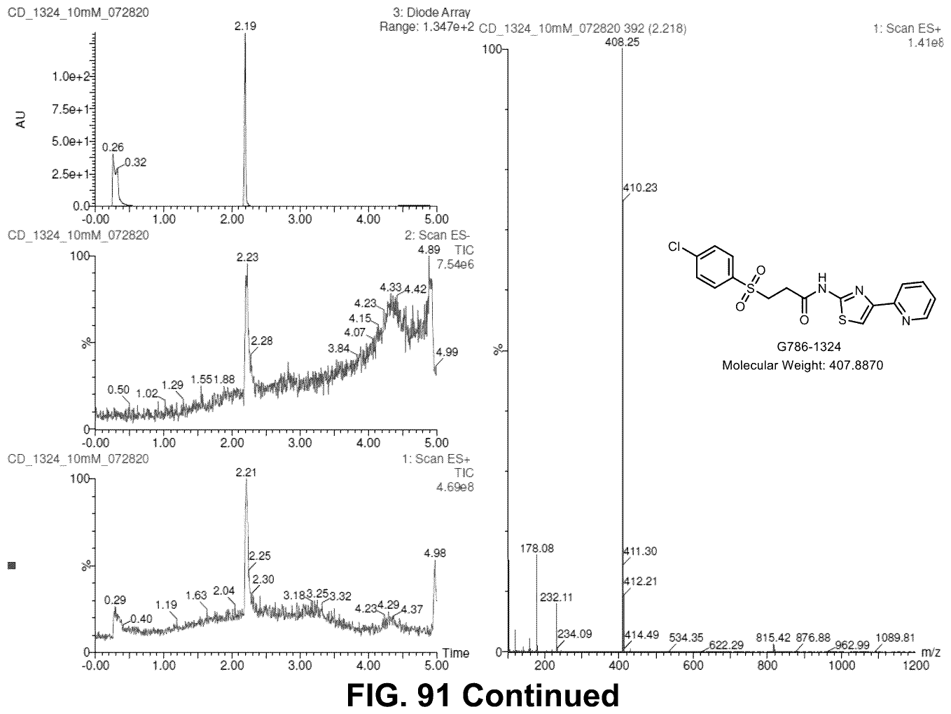

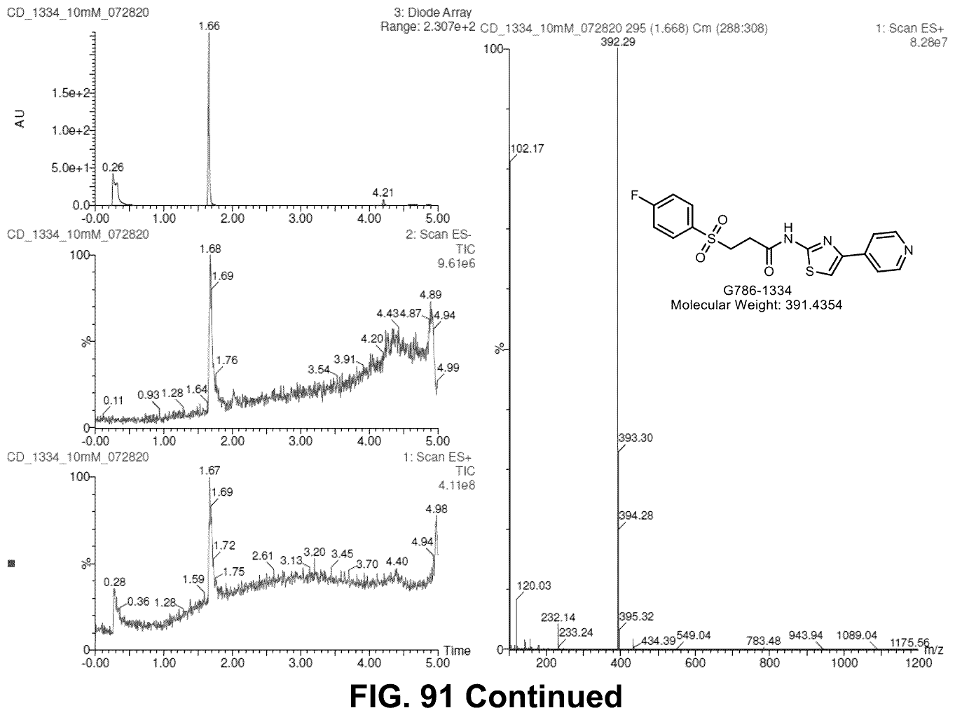

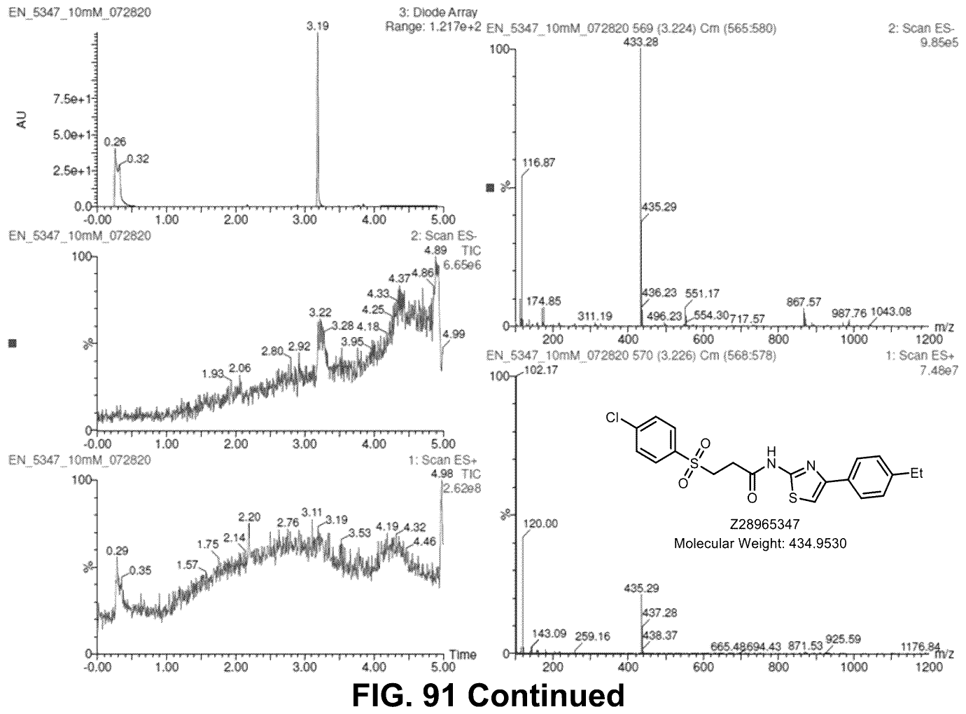

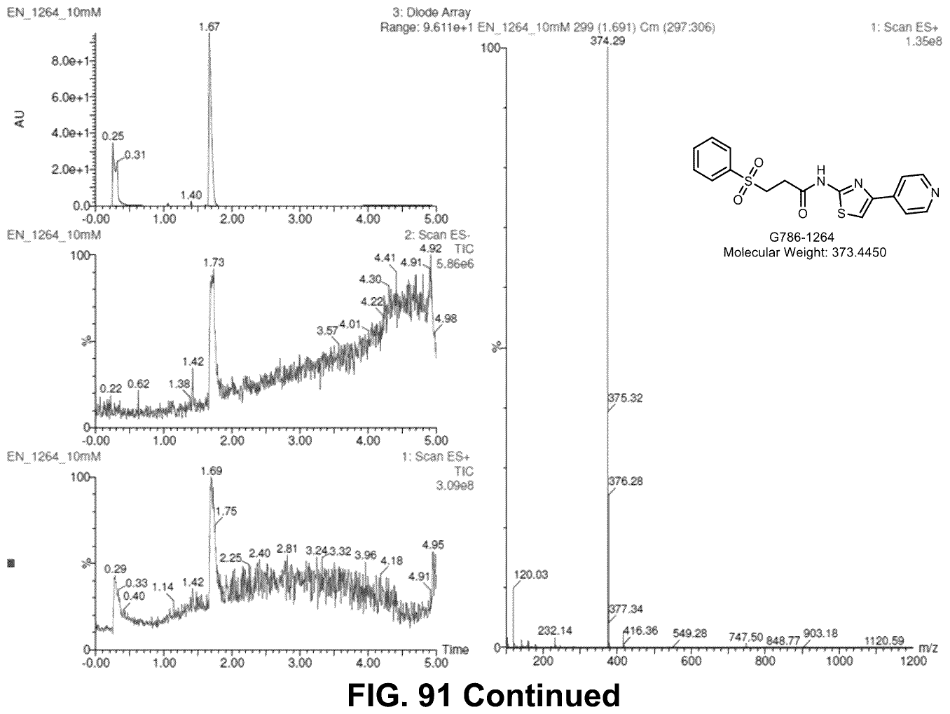

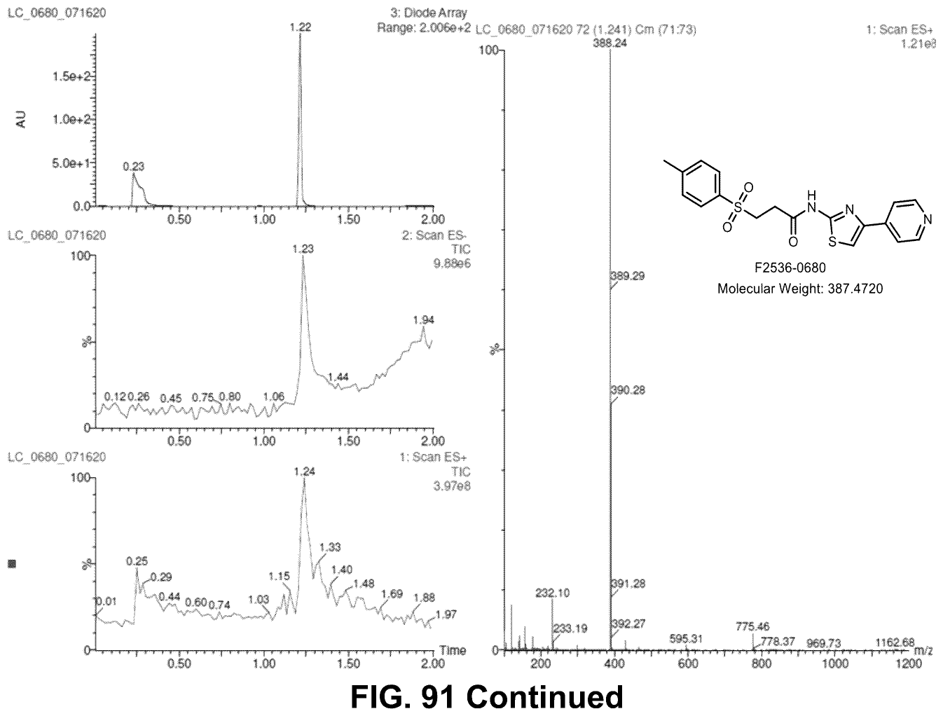

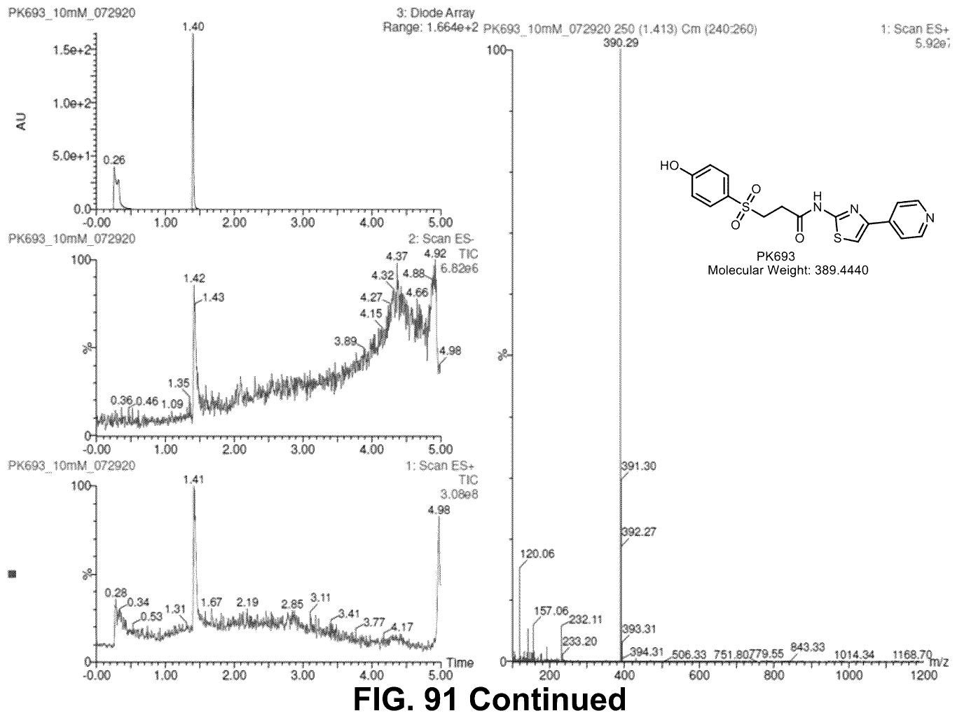

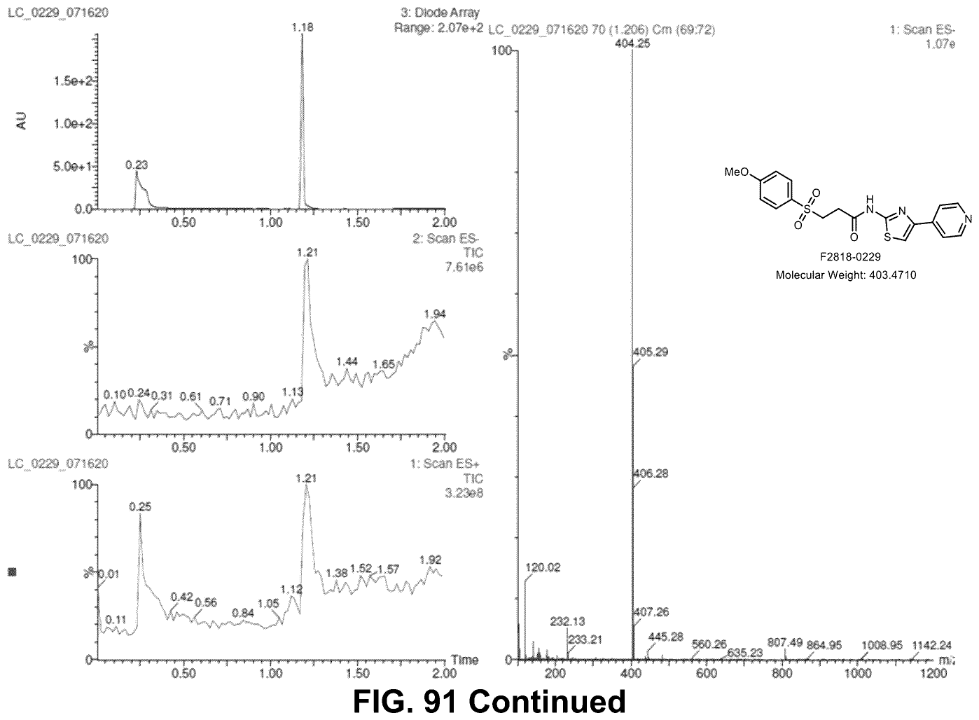

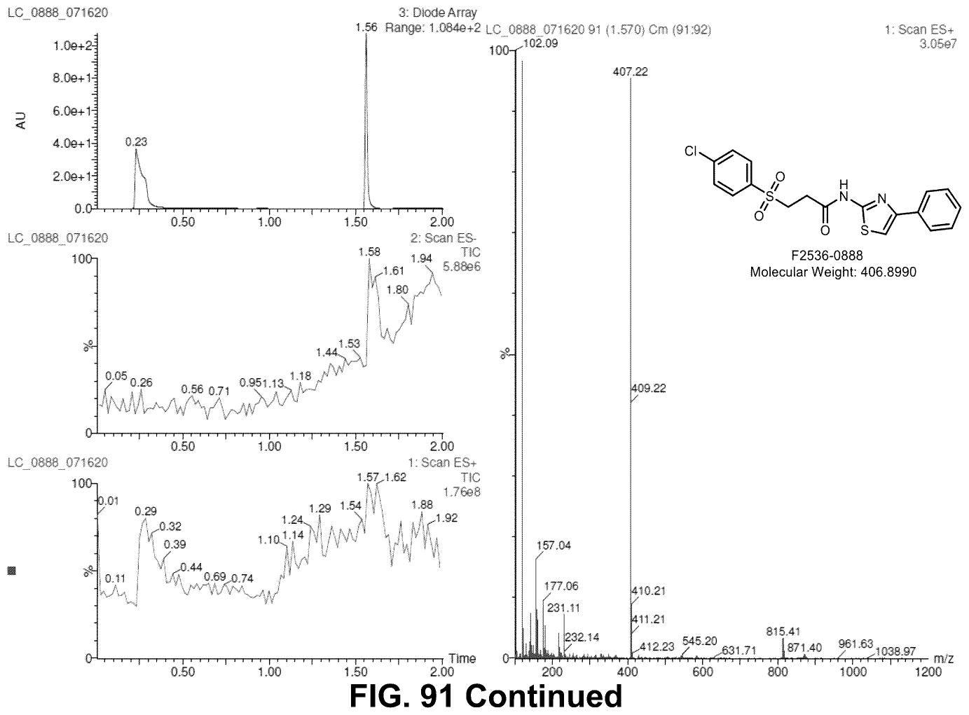

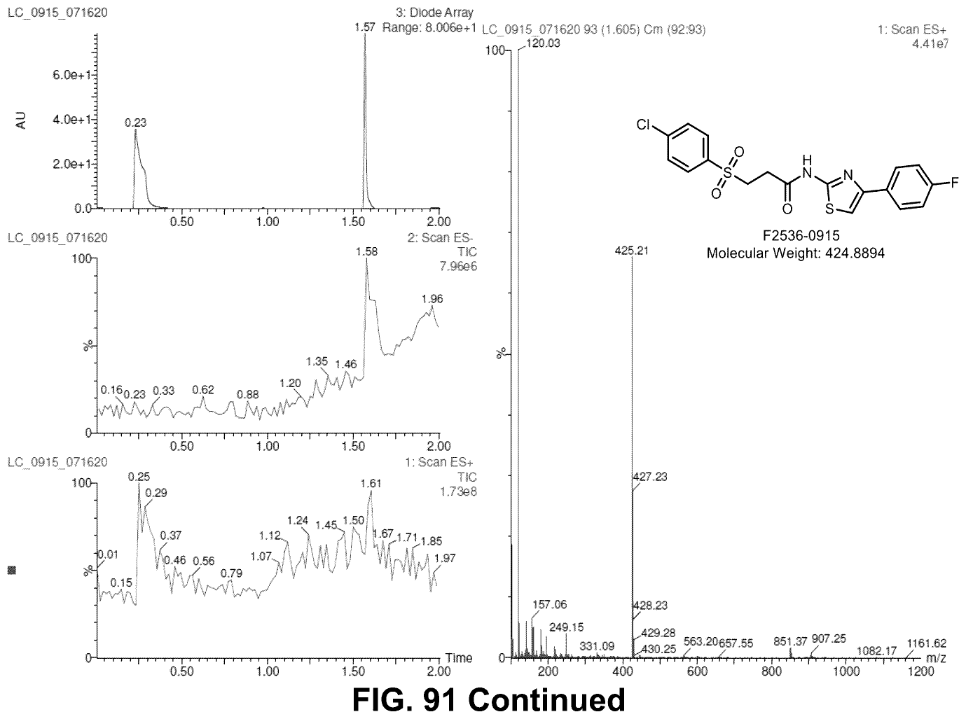

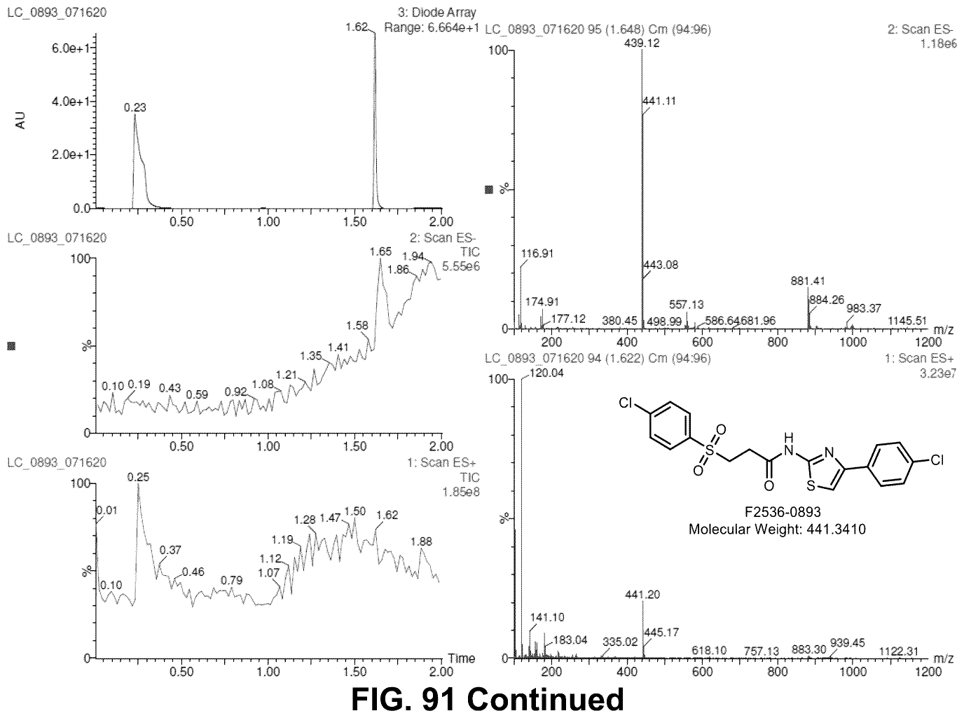

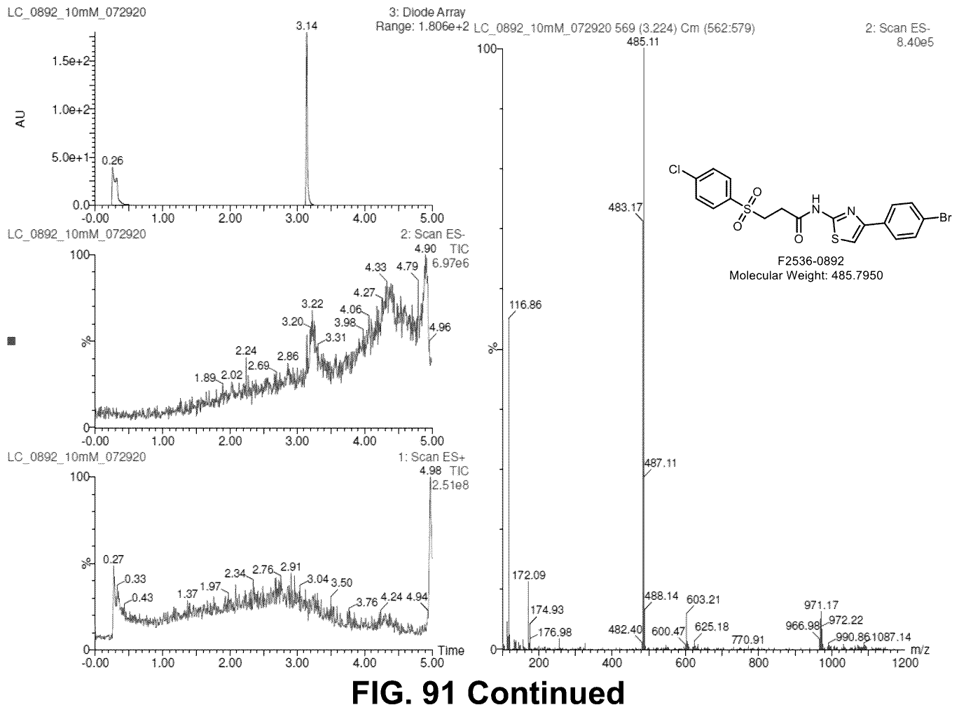

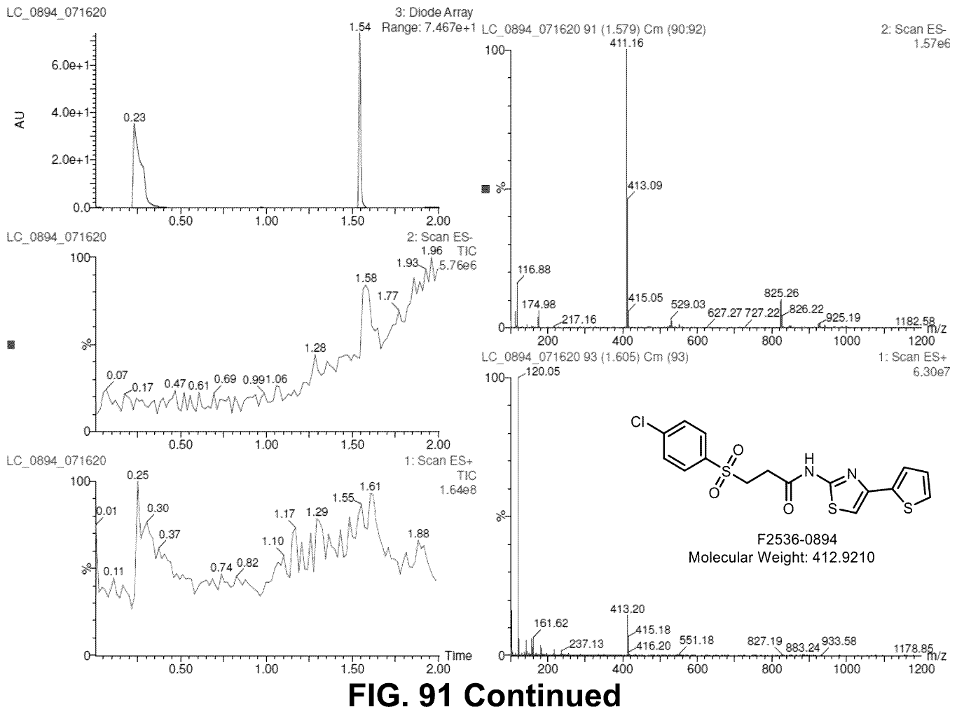

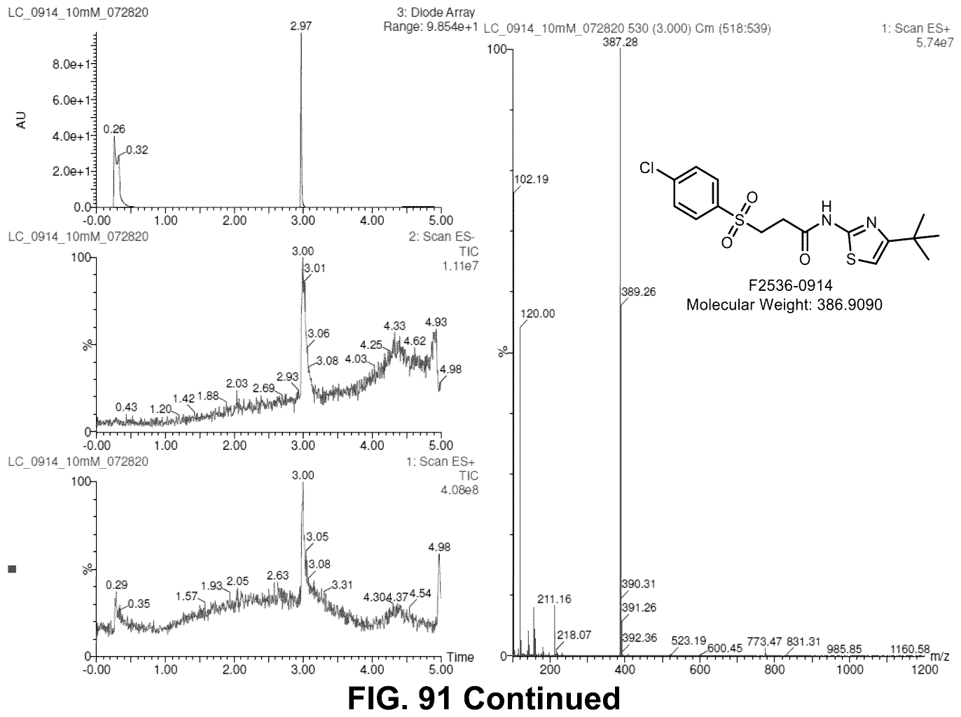

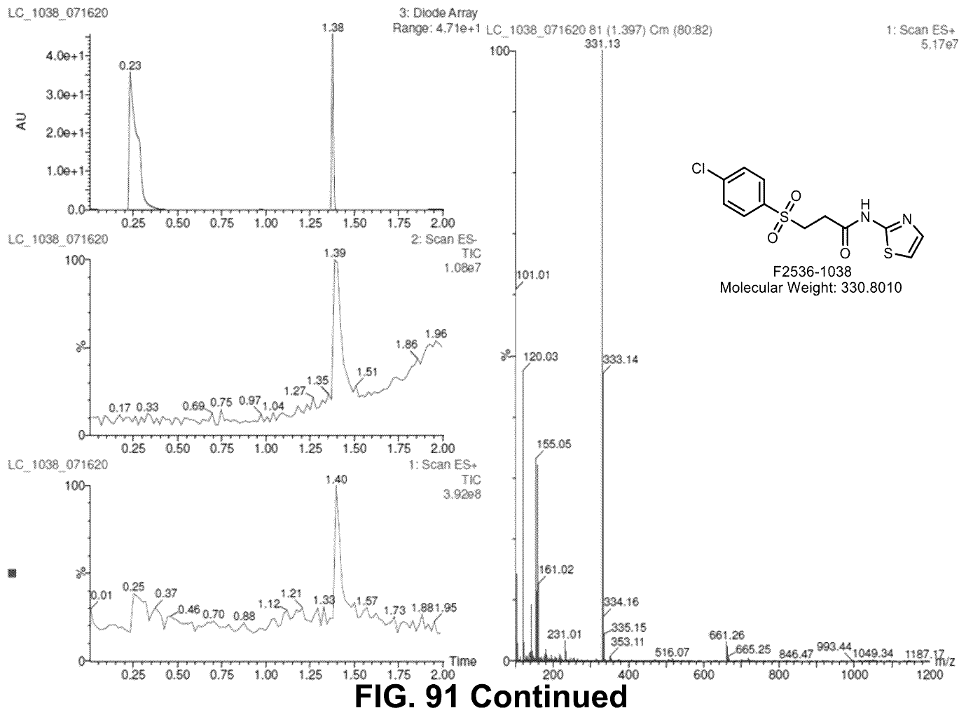

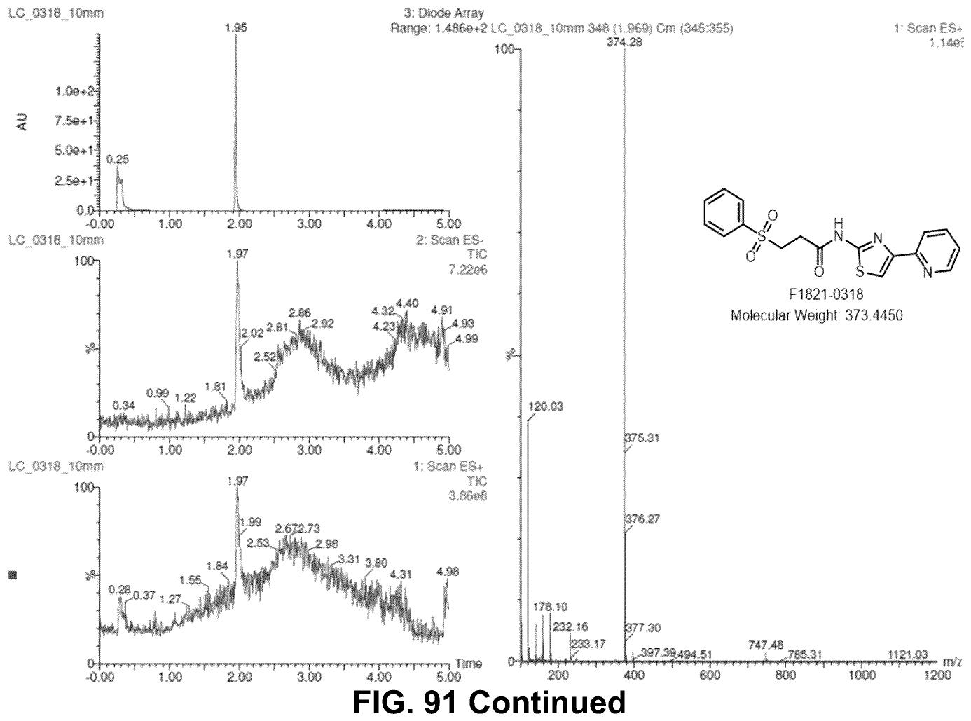

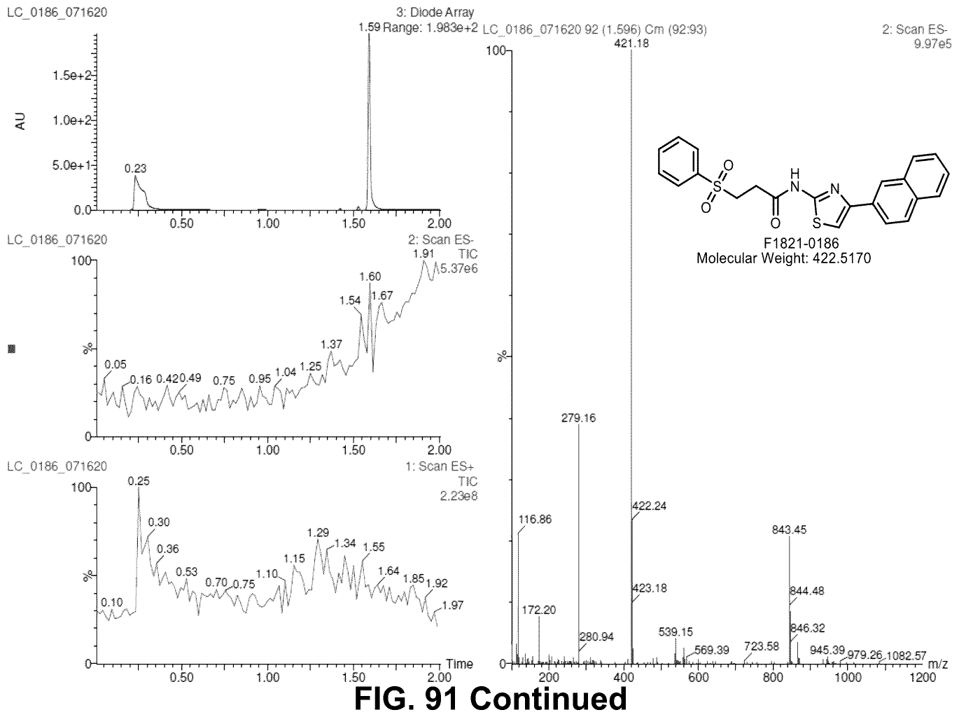

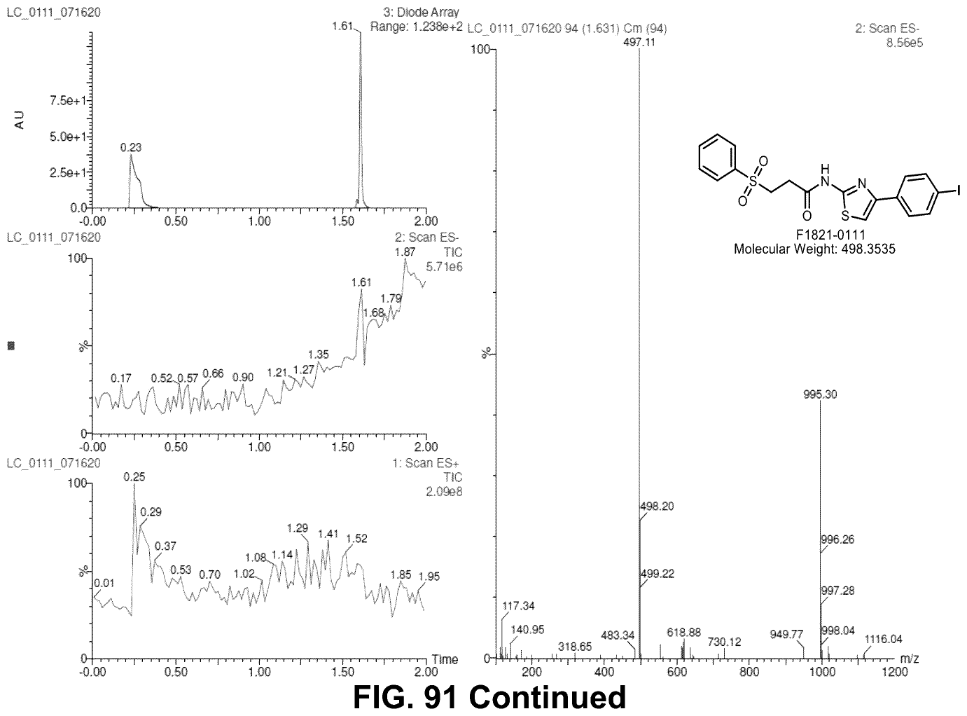

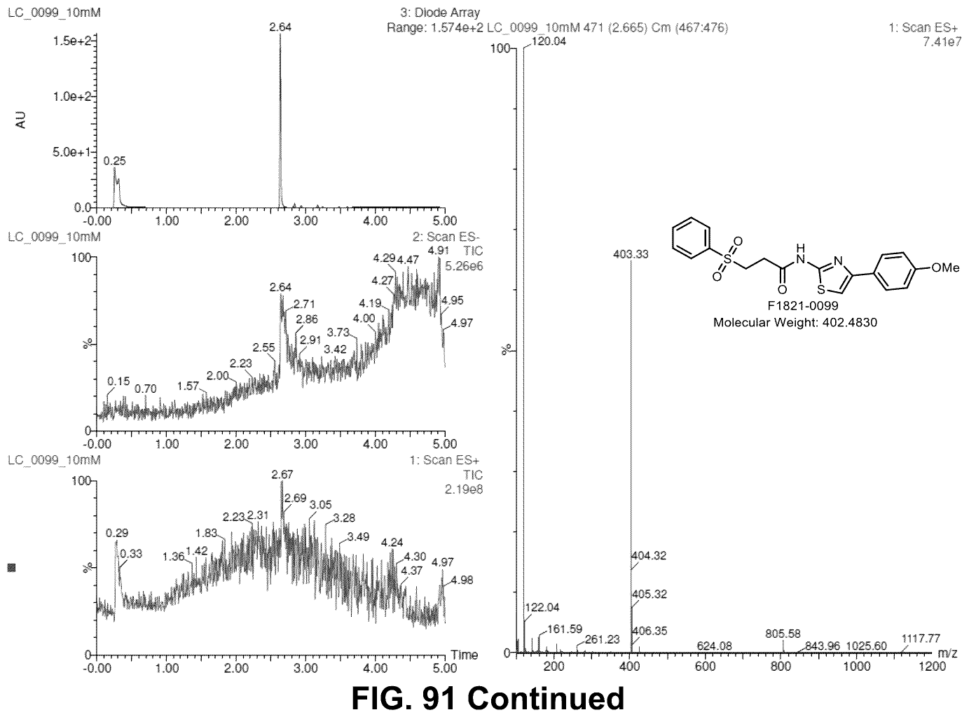

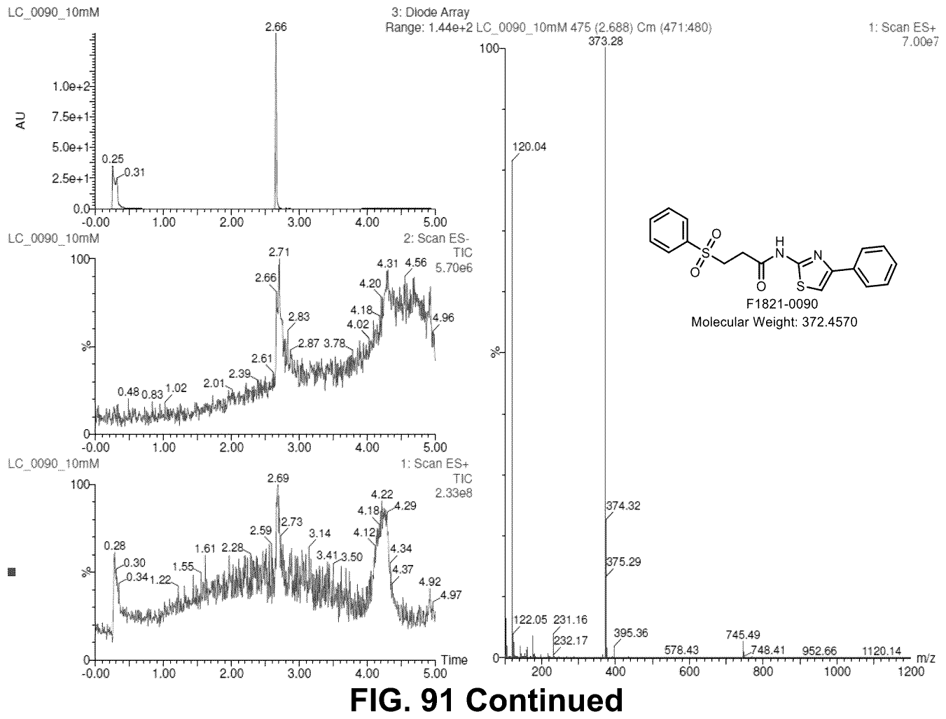

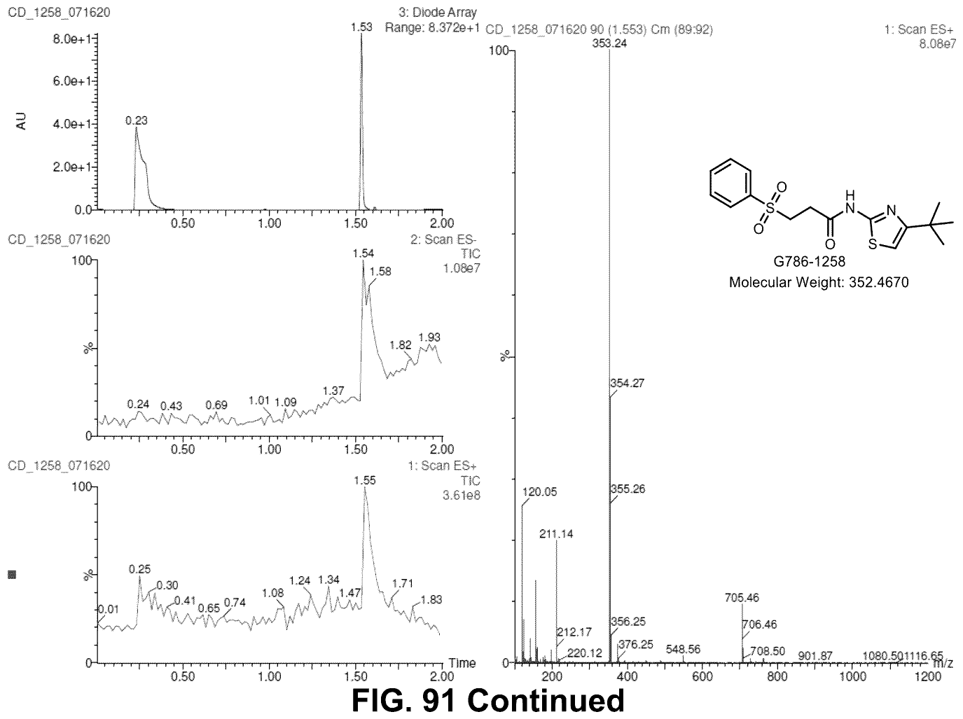

Based on SAR studies, Applicants designed an inactive analog of BRD7586 containing a bulky bromophenyl group and thioether that can serve as a control. This analog (BRD0033) was inactive in both the eGFP-disruption and HiBiT knock-in assays (FIG. 68A,D,E). Additionally, another analogue (F2537-0908) containing a thioether linkage instead of the sulfonyl group, but with the other substituent unchanged from BRD7586, had significantly lower inhibitory activity in both the eGFP-disruption and HiBiT knock-in assays (FIG. 76A-C).

Finally, Applicants performed early studies towards understanding the molecular mechanism of inhibition. The previously reported SpCas9:PAM interaction assay¹⁷ showed that BRD7586 does not inhibit the binding between SpCas9:gRNA complex and DNA (FIG. 76D), further suggesting that BRD7586 potentially disrupts SpCas9 catalysis. Applicants also performed early studies towards binding pocket identification using photocrosslinked SpCas9:gRNA and diazirine-BRD7586, after which an acid-cleavable and isotope-coded biotin-azide (FIG. 75C) was appended to the conjugate via click chemistry (FIG. 75B). Streptavidin pull-down followed by tryptic digestion left only the crosslinked peptides on the bead surface, and acid-mediated cleavage released the cross-linked peptides with the unique isotope tag identified using mass spectrometry (FIG. 75D). After confirming the isotope patterns using MS1 and the fragmentation patterns using MS2, Applicants identified peptides that crosslinked with BRD7586 (FIG. 75E, F and Table 14). Applicants also performed docking studies using Schrödinger Maestro v12.1 in the region of the photo-crosslinked sites (PDB: 5F9R).³⁸ These results suggest that the inhibitor may bind between the HNH nuclease and the helical recognition domains (FIG. 75G), although additional studies are needed to experimentally confirm these computational results.

### Discussion

Here Applicants report a universal platform to identify inhibitors of CRISPR-associated nucleases and demonstrate its usefulness by identifying a potent small-molecule inhibitor of SpCas9. Addressing issues in previous assay formats that bottlenecked the inhibitor discovery process, the platform is broadly applicable across multiple nuclease families and can report on the inhibition of any stage in the catalytic process. For example, the CAA for CRISPR-associated nucleases enabled the interrogation of all aspects of catalysis such as DNA binding, protein conformational changes, and DNA cleavage, allowing a higher chance of inhibitor discovery. Furthermore, CAA can be used for both Cas9 and Cas12a even though they have a relatively different mode of catalysis, and Applicants expect that CAA would be readily adapted for emerging CRISPR-associated nucleases. Logical computation capabilities can be added to the CAA setup using DNA logic circuits.^{39, 40} Applicants also demonstrate a robust and rapid workflow to verify cellular activities of numerous hits from the CAA, which involves a fluorescence-imaging-based eGFP disruption assay and a luminescence-based HiBiT knock-in assay. Because these high-throughput assays are completely orthogonal, the platform allows reliable identification of the final lead compound with minimal resources and time.

A potent small-molecule inhibitor of SpCas9 identified from the workflow, BRD7586, exhibited inhibitory activity in all explored genome-editing scenarios. Particularly, BRD7586 inhibited genome editing at diverse endogenous loci regardless of the delivery methods of the genome-editing machinery (i.e., plasmid or RNP). Moreover, treatment with BRD7586 improved the specificity of genome editing at diverse genomic loci, demonstrating its immediate usefulness for precise genome editing. Since small molecules are readily cell-permeable, BRD7586 will complement anti-CRISPR proteins in therapeutic genome editing. SAR studies demonstrated the specific nature of the interaction between BRD7586 and the SpCas9 ribonucleoprotein complex. While BRD0539 possesses a complex tetrahydroquinoline core requiring 8 synthetic steps, several of which are challenging,¹⁷ BRD7586 possesses a simple core that can be accessed in a single step from commercially available materials. Owing to ease of synthesis, Applicants envision that BRD7586 could serve as a starting point for more potent inhibitors and degraders of SpCas9.^{43, 44} For example, proteolysis targeting chimeras (PROTACs)^{43, 44} could be generated by joining the inhibitor to the ubiquitin ligase binder to cause the degradation of SpCas9. Overall, the reported anti-CRISPR molecules highlight that chemical approaches can control and enhance the capabilities of CRISPR-based technologies and are an important step towards their dose and temporal control. These studies have the potential to impact wide-ranging areas in basic and biomedical sciences and biotechnology.

### References for Example 2

1. Fellmann, C., Gowen, B. G., Lin, P. C., Doudna, J. A. & Corn, J. E. Cornerstones of CRISPR-Cas in drug discovery and therapy. Nat. Rev. Drug Discov. 16, 89-100 (2017).
2. Gangopadhyay, S. A. et al. Precision Control of CRISPR-Cas9 Using Small Molecules and Light. Biochemistry 58, 234-244 (2019).
3. Kang, S. H. et al. Prediction-based highly sensitive CRISPR off-target validation using target-specific DNA enrichment. Nat. Commun. 11(2020).
4. Cox, D. B. T., Platt, R. J. & Zhang, F. Therapeutic genome editing: prospects and challenges. Nat. Med. 21, 121 (2015).
5. Modell, A. E., Siriwardena, S. U., Shoba, V. M., Li, X. & Choudhary, A. clustered regularly interspaced short palindromic repeat-Chemical and optical control of CRISPR-associated nucleases. Curr. Opin. Chem. Biol. 60, 113-121 (2020).
6. Davidson, A. R. et al. Anti-CRISPRs: Protein Inhibitors of CRISPR-Cas Systems. Annu. Rev. Biochem. 89, 309-332 (2020).
7. Marino, N. D., Pinilla-Redondo, R., Csorgo, B. & Bondy-Denomy, J. Anti-CRISPR protein applications: natural brakes for CRISPR-Cas technologies. Nat. Methods 17, 471-479 (2020).
8. Yu, L. & Marchisio, M. A. Types I and V Anti-CRISPR Proteins: From Phage Defense to Eukaryotic Synthetic Gene Circuits. Front. Bioeng. Biotechnol. 8, 575393 (2020).
9. O'Connor, C. J., Laraia, L. & Spring, D. R. Chemical genetics. Chem Soc Rev 40, 4332-4345 (2011).
10. Walsh, D. P. & Chang, Y. T. Chemical genetics. Chem. Rev. 106, 2476-2530 (2006).
11. Burslem, G. M. & Crews, C. M. Small-Molecule Modulation of Protein Homeostasis. Chem. Rev. 117, 11269-11301 (2017).
12. Jiang, F. & Doudna, J. A. CRISPR-Cas9 Structures and Mechanisms. Annu. Rev. Biophys. 46, 505-529 (2017).
13. Stella, S., Alcon, P. & Montoya, G. Structure of the Cpf1 endonuclease R-loop complex after target DNA cleavage. Nature 546, 559-563 (2017).
14. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat. Biotechnol. 34, 339-344 (2016).
15. Zhu, X. et al. Cryo-EM structures reveal coordinated domain motions that govern DNA cleavage by Cas9. Nat. Struct. Mol. Biol. 26, 679-685 (2019).
16. Huai, C. et al. Structural insights into DNA cleavage activation of CRISPR-Cas9 system. Nat. Commun. 8, 1375 (2017).
17. Maji, B. et al. A High-Throughput Platform to Identify Small-Molecule Inhibitors of CRISPR-Cas9. Cell 177, 1067-1079.e1019 (2019).
18. Koehler, A. N. A complex task? Direct modulation of transcription factors with small molecules. Curr. Opin. Chem. Biol. 14, 331-340 (2010).
19. Seamon, K. J., Light, Y. K., Saada, E. A., Schoeniger, J. S. & Harmon, B. Versatile High-Throughput Fluorescence Assay for Monitoring Cas9 Activity. Anal. Chem. 90, 6913-6921 (2018).
20. Yurke, B., Turberfield, A. J., Mills, A. P., Jr., Simmel, F. C. & Neumann, J. L. A DNA-fueled molecular machine made of DNA. Nature 406, 605-608 (2000).
21. Srinivas, N. et al. On the biophysics and kinetics of toehold-mediated DNA strand displacement. Nucleic Acids Res. 41, 10641-10658 (2013).
22. Dong, D. et al. Structural basis of CRISPR-SpyCas9 inhibition by an anti-CRISPR protein. Nature 546, 436-439 (2017).
23. Yang, H. & Patel, D. J. Inhibition Mechanism of an Anti-CRISPR Suppressor AcrIIA4 Targeting SpyCas9. Mol. Cell 67, 117-127.e115 (2017).
24. Forsberg, K. J. et al. Functional metagenomics-guided discovery of potent Cas9 inhibitors in the human microbiome. eLife 8, e46540 (2019).
25. Dillard, K. E. et al. Mechanism of broad-spectrum Cas9 inhibition by AcrIIA11. bioRxiv, 10.1101/2021.09.15.460536 (2021).
26. Nishimasu, H. et al. Crystal Structure of Cas9 in Complex with Guide RNA and Target DNA. Cell 156, 935-949 (2014).
27. Nishimasu, H. et al. Crystal Structure of Staphylococcus aureus Cas9. Cell 162, 1113-1126 (2015).
28. Swarts, D. C. & Jinek, M. Mechanistic Insights into the cis- and trans-Acting DNase Activities of Cas12a. Mol. Cell 73, 589-600.e584 (2019).
29. Swarts, D. C., van der Oost, J. & Jinek, M. Structural Basis for Guide RNA Processing and Seed-Dependent DNA Targeting by CRISPR-Cas12a. Mol. Cell 66, 221-233.e224 (2017).
30. Chen, J. S. et al. CRISPR-Cas12a target binding unleashes indiscriminate single-stranded DNase activity. Science 360, 436-439 (2018).
31. Watters, K. E., Fellmann, C., Bai, H. B., Ren, S. M. & Doudna, J. A. Systematic discovery of natural CRISPR-Cas12a inhibitors. Science 362, 236-239 (2018).
32. Zhang, J. H., Chung, T. D. & Oldenburg, K. R. A Simple Statistical Parameter for Use in Evaluation and Validation of High Throughput Screening Assays. J. Biomol. Screen. 4, 67-73 (1999).
33. Fu, Y. et al. High-frequency off-target mutagenesis induced by CRISPR-Cas nucleases in human cells. Nat. Biotech. 31, 822-826 (2013).
34. Lim, D. et al. Engineering designer beta cells with a CRISPR-Cas9 conjugation platform. Nat. Commun. 11, 4043 (2020).
35. Flaxman, H. A., Chang, C. F., Wu, H. Y., Nakamoto, C. H. & Woo, C. M. A Binding Site Hotspot Map of the FKBP12-Rapamycin-FRB Ternary Complex by Photoaffinity Labeling and Mass Spectrometry-Based Proteomics. J. Am. Chem. Soc. 141, 11759-11764 (2019).
36. Miyamoto, D. K., Flaxman, H. A., Wu, H. Y., Gao, J. & Woo, C. M. Discovery of a Celecoxib Binding Site on Prostaglandin E Synthase (PTGES) with a Cleavable Chelation-Assisted Biotin Probe. ACS Chem. Biol. 14, 2527-2532 (2019).
37. Joiner, C. M., Levine, Z. G., Aonbangkhen, C., Woo, C. M. & Walker, S. Aspartate Residues Far from the Active Site Drive O-GlcNAc Transferase Substrate Selection. J. Am. Chem. Soc. 141, 12974-12978 (2019).
38. Jiang, F. et al. Structures of a CRISPR-Cas9 R-loop complex primed for DNA cleavage. Science 351, 867-871 (2016).
39. Seelig, G., Soloveichik, D., Zhang, D. Y. & Winfree, E. Enzyme-Free Nucleic Acid Logic Circuits. Science 314, 1585-1588 (2006).
40. Qian, L. & Winfree, E. Scaling Up Digital Circuit Computation with DNA Strand Displacement Cascades. Science 332, 1196-1201 (2011).
41. Sternberg, S. H., LaFrance, B., Kaplan, M. & Doudna, J. A. Conformational control of DNA target cleavage by CRISPR-Cas9. Nature 527, 110-113 (2015).
42. Dagdas, Y. S., Chen, J. S., Sternberg, S. H., Doudna, J. A. & Yildiz, A. A conformational checkpoint between DNA binding and cleavage by CRISPR-Cas9. Sci. Adv. 3, eaa00027 (2017).
43. Bondeson, D. P. et al. Lessons in PROTAC design from selective degradation with a promiscuous warhead. Cell Chem. Biol. 25, 78-87.e75 (2018).
44. Lai, A. C. & Crews, C. M. Induced protein degradation: an emerging drug discovery paradigm. Nat. Rev. Drug Discov. 16, 101-114 (2017).
45. Charlesworth, C. T. et al. Identification of preexisting adaptive immunity to Cas9 proteins in humans. Nat. Med. 25, 249-254 (2019).
46. Wagner, D. L. et al. High prevalence of Streptococcus pyogenes Cas9-reactive T cells within the adult human population. Nat. Med. 25, 242-248 (2019).
47. Sreekanth, V. et al. Chemogenetic System Demonstrates That Cas9 Longevity Impacts Genome Editing Outcomes. ACS Central Sci. 6, 2228-2237 (2020).
48. Nobrega, F. L., Costa, A. R., Kluskens, L. D. & Azeredo, J. Revisiting phage therapy: new applications for old resources. Trends Microbiol. 23, 185-191 (2015).

### Methods

In Vitro Transcription of gRNA.

Linear DNA fragments containing the T7 RNA polymerase promoter sequence upstream of the desired gRNA protospacer and the gRNA backbone were generated by PCR (Q5 Hot Start MasterMix, New England Biolabs) using the primers listed in Table 10. The fragments were concentrated on MinElute columns (Qiagen). The gRNA was transcribed with the HiScribe T7 High Yield RNA Synthesis Kit (New England Biolabs) at 37° C. for 14-16 h with 400 ng of linear template per 30 μL of reaction. gRNA was purified using the MEGAClear Transcription Clean Up Kit (Thermo Fisher) according to the manufacturer's instructions. Purified gRNAs were stored in aliquots at −80° C.

### Generation of Cumulative Activity Assay Substrates.

Oligo-annealing solutions were prepared by mixing complementary strands (10 μM final concentration) together in 1× Cas9 assay buffer (20 mM Tris-HCl, pH=7.5, 100 mM KCl, 5 mM MgCl₂). Oligonucleotides were annealed by heating to 95° C. for 5 min, followed by slow cooling to 25° C. at a rate of 0.1° C./s to produce a double-stranded oligonucleotide. Complementary strands were purchased from Integrated DNA Technologies.

### Fluorescence Polarization Assay for Optimization of Substrates of SaCas9 and FnCas12a (FIG. 69B-D).

Fluorescence polarization assay for SaCas9 and FnCas12a was performed using the reported method with the substrates mentioned in the Table 8.¹⁷

### Optimization of Cumulative Activity Assay.

First, Applicants optimized various components and conditions of the strand displacement assay for Cas enzyme activity and generality with different Cas such as SpCas9 (FIG. 62B) or SaCas9 (FIG. 69F) or FnCas12a (FIG. 70A,B). Typically, SpCas9: gRNA (1:1.2 ratio) RNP was pre-formed at 1 μM in Cas9 assay buffer (20 mM Tris-HCl, pH=7.5, 100 mM KCl, 5 mM MgCl₂) for 5 min at 4° C. before diluting to 10 nM in Cas9 assay buffer (2× final concentration). 25 μL of the Cas9 2× stock was manually dispensed to a black 384-well plate (Corning 3575) using electronic pipette. Apo SpCas9 (without guide RNA) was used at the same concentration as a control for no activity (mock inhibition). Following this, pre-annealed Alexa-Fluor 647 labeled substrate and quencher were also diluted to 1 nM and 5 nM in Cas9 assay buffer, respectively (2× stock solution). Next, 25 μL of the substrate/quencher solution was added manually to each well of the Cas9-containing 384-well plates using an electronic pipette and was incubated at 37° C. for 2.5 h. Fluorescent signals were read with the microplate reader set to read Alexa-Fluor 647 fluorescence. Assay was performed similarly with SaCas9 and FnCas12a using the respective enzyme, gRNA, substrate, and quencher oligos. Applicants similarly divulged the effect of PAM sequences of dsDNA on its assay specificity using TGG, TGC, AAC PAM containing substrates for SpCas9 (FIG. 62C), and ACGGGT, ACGGTT, TGCCCA PAM substrate for SaCas9 (FIG. 62F) and TTTC, TTGC, AAAG PAM substrates for FnCpf1 (FIG. 63B-C). Applicants also optimized the effective concentration of DS-AF647 and SS-AF647 fluorophores (FIG. 64A), DS-substrate to SpCas9 RNP ratio (FIG. 64B) and substrate to quencher ratio (FIG. 64C) on assay performance. Similarly, Applicants performed a time course experiment to establish the time required for maximum completion of the reaction at various substrate to SpCas9 RNP ratios (FIG. 64D).

### Validation of Cumulative Activity Assay.

Applicants further validated the strand displacement assay using protein inhibitors such as AcrIIA4 (FIG. 62E), AcrIIA11 (FIG. 69E), AcrVA1 (FIG. 63E) that inhibit Cas enzymes by different mechanisms. Typically, active SpCas9: gRNA (1:1.2 ratio) RNP was pre-formed at 1 μM in Cas9 assay buffer for 5 min at 4° C. before diluting to 50 nM in Cas9 assay buffer (10× final concentration). 25 μL of the Cas9 2× stock was manually dispensed to a black 384-well plate (Corning 3575) using electronic pipette. Apo SpCas9 was used at the same concentration as a control for no activity. Various concentrations (final concentration of 10 μM to 0.61 nM) of 10× AcrIIA4 or AcrIIA11 along with buffer controls were manually added using electronic pipette and were incubated with SpCas9 for at least 30 min at room temperature. Following this, pre-annealed Alexa-Fluor 647 labeled substrate and quencher were also diluted to 10 nM and 50 nM in Cas9 assay buffer, respectively (10× stock solution). Next, 25 μL of the substrate/quencher solution was added manually to each well of the Cas9-containing 384-well plates using an electronic pipette and was incubated at 37° C. for 2.5 h. Fluorescent signals were read with the microplate reader set to read Alexa-Fluor 647 fluorescence. Similarly strand displacement assay for FnCas12a was validated using its protein inhibitor AcrVA1.

### Cumulative Activity Assay.

High-throughput screening with the strand displacement assay was performed as follows. Active SpCas9: gRNA (1:1.2 ratio) RNP was pre-formed at 1 μM in Cas9 assay buffer for 5 min at 4° C. before diluting to 10 nM in Cas9 assay buffer (2× final concentration). Using a liquid handling dispenser, 25 μL of the Cas9 2× stock was dispensed to a black 384-well plate (Corning 3575). Apo SpCas9 was used at the same concentration as a control for no activity. Compound libraries and DMSO controls were added via pin transfer of 100 nL from 10 mM or 5 mg/mL stocks in DMSO and were incubated with SpCas9 for at least 30 min at room temperature. Compound autofluorescence was measured at this time using a microplate reader (Envision, PerkinElmer) set to read Alexa-Fluor 647 fluorescence. Following this, pre-annealed Alexa-Fluor 647 labeled substrate and quencher were also diluted to 1 nM and 5 nM in Cas9 assay buffer, respectively (2× stock solution). Next, 25 pL of the substrate/quencher solution was added to each well of the Cas9-containing 384-well plates using a liquid handling dispenser and was incubated at 37° C. for 2.5 h. Fluorescent signals were read with the microplate reader set to read Alexa-Fluor 647 fluorescence. Compounds were screened in duplicate; data were processed to calculate the Z-score ([x−μ]/σ) values. Potential hit compounds (Z-score >3) were prioritized for further screening. Some compounds exhibit normalized Inhibition less than 0 (FIG. 64F), which arises from the quenching of the Alexa-Fluor 647 fluorescence as measured from the above counter screening. Those compounds were excluded from the further testing.

Gel-Monitored Cleavage Assays with FAM Oligos.

For SpCas9, RNP complex was formed by mixing SpCas9 and Spinach-targeting gRNA at room temperature for 15 minutes with a ratio of 1:1.2. Next, FAM-labeled dsDNA substrates were added to the mixture to give a final 30 μL solution of 20 nM FAM-dsDNA, 100 nM SpCas9, and 120 nM gRNA. The mixture was incubated at 37° C. for 3 h, resolved by 4-20% acrylamide gel, and imaged by an Azure 600 (Azure Biosystem) under the blue fluorescence channel. The same reaction conditions were used for SaCas9, AsCas12, LbCas12a, and FnCas12a. Native acrylamide gel electrophoresis (FIG. 62D) or urea-based denaturing gel electrophoresis (FIG. 62G, 63D, 69G, 70C) was performed for resolving the reaction mixtures.

Cell culture. U2OS.eGFP.PEST cells (gift from Prof. J. Keith Joung's lab) and HEK293T cells (ATCC #CRL-3216) were maintained in Dulbecco's Modified Eagle Medium supplemented with 10% fetal bovine serum, 1× penicillin/streptomycin, and 1 mM pyruvate. Cells were routinely tested for mycoplasma contamination using the Universal Mycoplasma Detection Kit (ATCC). None of the cell line was authenticated. eGFP disruption assay.

For SAR studies and dose-response studies, 300,000 U2OS.eGFP.PEST cells were nucleofected with 300 ng of SpCas9 plasmid (Addgene #43861) and 30 ng of eGFP-targeting gRNA plasmid (Addgene #47511)³³ using SE Cell Line 4D-Nucleofector X Kit (Lonza) following the pulse program of DN-100. For RNP-based genome editing, 10 pmol of SpCas9 (GenScript #Z03385) and 12 pmol of gRNA were mixed and incubated for 5 min. For RNP-based genome editing with LbCas12a, 15 pmol of LbCas12a (New England Biolabs #M0653T) and 20 pmol of crRNA (spacer: cgtcgccgtccagctcgacc) was used due to its lower basal activity. Cells were nucleofected with the resulting RNP complex using the same pulse program. Cells were transferred to a 96-well plate at the density of 25,000 cells per well, and incubated with indicated amount of compounds for 24 h. Cells were then fixed with 4% paraformaldehyde solution in PBS, and nuclei were stained by HCS NuclearMask Blue stain (Invitrogen). Imaging was performed using an ImageXpress Micro High-Content Analysis System (Molecular Devices) or an Operetta CLS High-Content Analysis System (PerkinElmer). Data analysis was performed using MetaXpress (Molecular Devices) or Operetta Harmony 4.8 (PerkinElmer). For the secondary screening assay, compounds were first dispensed to a 384-well plate using a Hewlett Packard D300e and resuspended in 25 μL of medium. Then, 5,000 nucleofected cells were added to each well in duplicate to give the final compound concentration of 20 μM. Cells were incubated for 24 h and imaging was performed. Transfection with SpCas9 plasmid only served as a positive control representing 100% inhibition, and transfection with SpCas9 and gRNA plasmids and treatment with DMSO served as a negative control. Z scores ((x−μ)/σ, where x is the signal from the sample, μ and σ are average and standard deviation from the negative controls) for each compound were calculated, and compounds showing Z score higher than 2 were selected and validated in additional orthogonal cellular assays.

### HiBiT Knock-In Assay.

Approximately 400,000 HEK293T cells were nucleofected with 400 ng of SpCas9 plasmid, 40 ng of GAPDH-targeting gRNA plasmid, and 40 pmol of single-strand oligodeoxynucleotide (ssODN) using SF Cell Line 4D-Nucleofector X Kit (Lonza) following the pulse program of DS-150. For RNP-based genome editing, 10 pmol of Cas9 and 12 pmol of gRNA were mixed and incubated for 5 min. Then, 20 pmol of ssODN was added. Cells were nucleofected with the resulting mixture using the same pulse program. Cells were then transferred to a 96-well plate at the density of 35,000 cells per well, and incubated with indicated amount of compounds for 24 h. Cell viability was measured using PrestoBlue reagent (Thermo) with a SpectraMax M5 (Molecular Devices) at the excitation and emission wavelength of 544 nm and 590 nm, respectively. Next, luminescence measurement was performed using the Nano-Glo HiBiT Lytic Detection System (Promega) according to the manufacturer's protocol with an EnVision Multilabel Plate Reader (PerkinElmer) at the integration time of 0.5 s per well. The resulting luminescence signals were normalized based on the cell viability.³⁴

### Compound-SpCas9 Interaction in BLI.

The experiments were performed in a 96-well format with a 180 μL reaction volume using Biotin-BRD7586 and streptavidin sensors. To start, 1 μM of the biotinylated compound was loaded onto the sensors for 180 s in a 20 mM Tris buffer (100 mM KCl, 5 mM MgCl₂, 1 mM DTT, 0.01% Tween, pH 7.4). Compound-loaded sensors were then allowed to associate with different concentrations of the SpCas9:gRNA complex (0.15-1 μM) for 300 s followed by dissociation in reaction buffer. The reference sensor was loaded with compound and allowed to associate and dissociate in reaction buffer alone. Response curves were fitted with a 2:1 stoichiometric model, and a global fit steady-state analysis was performed using the manufacturer's protocol. Experiments were performed in triplicate. Control experiments were performed using a Biotin-PEG3-azide. In this experiment, streptavidin sensors were associated with 1 μM of biotin-PEG3-azide, 1 μM of Biotin-BRD7586 (FIG. 70D,E), or reaction buffer alone. The sensors were then allowed to associate with different concentrations of SpCas9:gRNA complex (0.15-1 μM) or buffer alone.

### STD NMR Binding Assay.

All samples were prepared with 20 μM of BRD7586 in a 20 mM Tris-dl 1 buffer (pH 7.4) in D₂O with or without 5 μM of SpCas9:gRNA in a 3 mm NMR tube. Experiments were performed on a 600 MHz (¹⁹F: 564.71 MHz) Bruker AVANCE III NMR spectrometer equipped with a 5 mm QCI-F CryoProbe and a SampleJet for automated sample handling. To acquire the spectra, a standard one-pulse STD experiment with WALTZ-16 for proton decoupling during acquisition, a 5 s recycle delay, and 256 scans were used. All spectra were recorded at 280 K. NMR data were apodized with a 1-Hz exponential function prior to Fourier transformation. All spectra were baseline corrected, and peak widths and intensities were extracted using the automated line-fitting feature provided with the MNova software package.

### Cell Viability Assay.

HEK293T cells or U2OS.eGFP.PEST cells were plated in a 96-well plate at the density of 30,000 cells per well or 20,000 cells per well, respectively. The next day, cells were treated with indicated amount of compounds for 24 h. Then, cellular ATP levels were measured using CellTiter-Glo Luminescent Cell Viability Assay (Promega) with an EnVision Multilabel Plate Reader (PerkinElmer) at the integration time of 0.5 s per well.

### Targeted Deep Sequencing to Detect Indels at Endogenous Loci.

U2OS.eGFP.PEST cells were nucleofected as described above for the eGFP disruption assay, plated in a 24-well plate at the density of 150,000 cells per well, and incubated with BRD7586 for 24 h. Then, the genomic DNA was extracted using DNeasy Blood & Tissue Kit (Qiagen). HEK293T cells were plated in a 24-well plate at the density of 100,000 cells per well. The next day, cells were transfected with 500 ng of SpCas9 plasmid and 250 ng of EMX1-, VEGFA-, or FANCF-targeting gRNA plasmid using Lipofectamine 3000 (Invitrogen). Indicated amount of compound was added at the time of transfection, cells were incubated for 24 h or 48 h, and genomic DNA was extracted. Next-generation sequencing (NGS) samples were prepared using a two-step PCR protocol. NGS libraries were quantified using KAPA Library Quantification Kit (Roche) and diluted to 4 nM. Sequencing of the pooled library was performed using MiSeq Reagent Kit v2 (Illumina). The percentage of indel in the demultiplexed sequence files was analyzed using the CRISPResso2 software from the Pinello Lab.⁴⁹

### Immunoblotting.

Approximately 500,000 U20S.eGFP.PEST cells were nucleofected as described above with 500 ng of SpCas9 plasmid. Then, cells were plated in a 12-well plate with indicated amount of compound, and incubated for 24 h. HEK293T cells were transfected with 500 ng SpCas9 plasmid as described above, and incubated with the compound for 24 h in a 24-well plate. Cells were harvested and lysed by RIPA buffer containing Protease Inhibitor Cocktail (Roche). Lysate was cleared by centrifugation at 20,000 g in 4° C., and the supernatant was taken to measure the protein concentration using BCA assay. Approximately 10-20 pg of the total protein was used for immunoblotting. Rabbit anti-SpCas9 (Abcam #89380, 1:1,000 dilution) and mouse anti-a-tubulin (CST #3873, 1:2,000 dilution) were used as primary antibodies. IRDye 680RD Donkey anti-Rabbit IgG (LI-COR #925-68073, 1:10,000 dilution) and IRDye 800CW Donkey anti-Mouse IgG (LI-COR #925-32212, 1:10,000 dilution) were used as secondary antibodies.

### Plasma Stability Assay.

The stability of the compound in mouse plasma was assessed following a reported protocol.⁵⁰ BRD7586 (2 μM) was incubated with 50% mouse plasma (K2 EDTA, BioIVT) in PBS for 2 h in duplicate. Propantheline was included as a control.

### In Vitro DNA Cleavage Assay.

The inhibition of SpCas9 nuclease activity was assessed in an in vitro DNA cleavage assay in PBS buffer with 10 mM MgCl₂·6H₂O in 50 μL reaction volume. First, Cas9:gRNA complex (30 nM Cas9 (NEB) and 36 nM eGFP targeting gRNA) was formed by mixing each component at a 1:1.2 (Cas9:gRNA) molar ratio and incubating at room temperature for 10 minutes. BRD7586 at doses 0, 5, 10, 20, 30, 40 μM were incubated with Cas9:gRNA complex at 37° C. for 30 minutes at 700 rpm. PCR amplified target eGFP DNA (2 nM) was added after 30 minutes of compound incubation and the mixture was incubated at 37° C. for 30 minutes at 700 rpm. Proteinase K (5 μL) was added and incubated at 37° C. for 30 minutes at 700 rpm to digest the Cas9. The resulting mixtures were purified PCR mini elute kit (Qiagen) and the eluted DNA was quantified by Qubit HS DNA quantification method. Equal amounts of DNA samples were run on a 1% agarose E-gels (invitrogen) for 7 minutes. Images were obtained by an Azure 600 (Azure Biosystem) and quantification of band intensities were performed by ImageJ based analysis.

### Photo-Crosslinking.

Cas9 RNP complex was formed by mixing Cas9 (1 μM) and the eGFP-targeting gRNA (1 μM) in a binding buffer (HEPES 20 mM, KCl 100 mM, pH 7.6) for 15 min. Next, BRD7586 (5 μM) was added to the mixture when competition is required (the last lane of FIG. 68B). Finally, Diazirine-BRD7586 was added (1 μM) and the mixture was incubated for 20 min at RT with a final reaction volume of 20 μL in a PCR tube. The mixture was irradiated with UV (365 nm) for 5 min on ice, then 2.5 μL of 10% RapiGest SF solution in PBS was added. Click chemistry was initiated by adding 100 μM of TAMRA-azide (Sigma #760757), 350 μM of Cu-TBTA, and 1.5 mM ascorbate with a final reaction volume of 27 μL. The reaction was conducted for 1 h at 30° C., then SDS-PAGE was performed immediately. The fluorescence gel scanning was conducted using an Azure 600 (Azure Biosystem) to detect the TAMRA fluorescence.

### LC-MS/MS Sample Preparation for Binding Site Identification.

Cas9 RNP complex was formed by mixing Cas9 (1 μM) and the eGFP-targeting gRNA (1 μM) in a binding buffer (HEPES 20 mM, KCl 100 mM, pH 7.6) for 15 min. Next, Diazirine-BRD7586 was added (1 μM) and the mixture was incubated for 20 min at RT with a final reaction volume of 20 μL in a PCR tube. Competitor was not used for this experiment. The mixture was irradiated with UV (365 nm) for 5 min on ice, then 2.5 μL of 10% RapiGest SF solution in PBS was added. Click chemistry was initiated by adding 100 μM of acid-cleavable biotin-azide tag, 350 μM of Cu-TBTA, and 1.5 mM ascorbate with a final reaction volume of 27 μL. The reaction was conducted for 1 h at 30° C. After the click chemistry, 4-fold volume of ice-cold methanol was added to the combined reaction mixture, and the final mixture was incubated overnight at −80° C. to induce protein precipitation. The protein was pelleted by centrifuging for 10 min at 16,100 g and 4° C. The supernatant was carefully discarded, and the resulting pellet was washed with methanol/PBS (4:1 v/v) and centrifuged for 10 min. After removal of the supernatant, the protein pellet was air-dried and resuspended in 400 μL of 1% RapiGest SF solution in PBS. The pellet was fully solubilized by brief sonication. Meanwhile, Streptavidin-agarose beads (200 μL of slurry, Invitrogen #SA10004) were washed three times with 1 mL PBS. Between the washes, the beads were pelleted by centrifugation (3,000 g, 3 min at 4° C.). Finally, the beads were suspended in 200 μL of PBS and mixed with the solubilized protein. The resulting mixture was incubated overnight at 4° C. with mild rotation. The beads were pelleted by centrifugation (3,000 g, 3 min at 4° C.), and the supernatant was discarded. The beads were washed once with 1 mL of 1% RapiGest SF solution in PBS, twice with 1 mL of 6 M urea solution in water, and twice with 1 ml PBS in succession. Between the washes, the beads were pelleted by centrifugation and the supernatant was removed. Next, the beads were resuspended in 200 μL of PBS, and the bound protein was reduced by adding 10 μL 5 mM DTT solution in PBS and incubating for 30 min at RT with rotation. The beads were pelleted by centrifugation, and washed once with 1 ml PBS. Next, the beads were suspended in 220 μL of 0.5 M urea solution in PBS. Then, 1.5 sg of trypsin (Promega #v5111) was added to the slurry of beads, and the resulting mixture was incubated for 16 h at 37° C. with rotation. The beads were pelleted by centrifugation, and the supernatant was collected. The beads were washed once with 200 μL of PBS and twice with 200 μL of water. The washed fraction was combined with the supernatant to form the ‘trypsin fraction’, concentrated to dryness using a Vacufuge plus (Eppendorf), and stored at −20° C. The biotin tag was cleaved by incubating the beads in 200 μL of 2% formic acid solution in water for 30 min at RT with rotation. The beads were pelleted by centrifugation, and the supernatant was collected in a 1.5-mL protein low-bind tube. This cleavage step was repeated once again, and the supernatant was combined. Then, the beads were washed twice, each time with 400 μL of washing solution (1% formic acid and 50% acetonitrile in water). The washing solution was combined with the above supernatant to form the ‘cleavage fraction’. This fraction was concentrated to dryness using the Vacufuge plus at 30° C. The dried cleavage fraction was resuspended in 50 μL of 1% formic acid solution in water. Next, desalting was performed using a ZipTip with 0.6 μL C18 resin (Millipore #ZTC18S). First, the tip was wet with methanol by pipetting three times, then equilibrated with 1% formic acid in water by pipetting three times. Sample was loaded on the tip by pipetting the dissolved peptide solution 20 times. The tip was washed with 50 μL of 1% formic acid in water. Next, peptides were eluted twice, each time with 50 μL of the elution solution (1% formic acid and 50% acetonitrile in water) into a 1.5-mL protein low-bind tube. The eluate was concentrated to dryness using the Vacufuge plus at 30° C., and stored at −20° C. until analysis.

### Structural Proteomics Mass Spectrometry.

The sample was separated on a 100 μm inner diameter microcapillary trapping column packed with approximately 3 cm of C18 Reprosil resin (5 μm, 100 Å, Dr. Maisch GmbH, Germany) and analytical column 50 cm microcappilarry based PharmaFluidics (Belgium) at 200 nL/min with a Lumos Tribrid Orbitrap (Thermo Scientific) equipped with Ultimate 3000 double nano HPLC pump (Thermo Scientific). The column temperature was maintained at 35° C. Peptides were eluted with a water/acetonitrile gradient (buffer A=0.1% formic acid/water, buffer B=0.1% formic acid/acetonitrile; flow rate 200 nL/min; gradient: hold at 2% B for 5 min, increase to 5% B over 1 min, increase to 40% B over 34 min, increase to 95% B over 5 min hold at 95% B for 15 min). Survey scans of peptide precursors were performed at 60K FWHM resolution over a m/z range of 400-1800. Tandem MS was performed on the most abundant precursors exhibiting a charge state from 2 to 4 with fragmentation energy of 35% for CID with an isolation window of 2 m/z and with fragmentation energy of 37% for HCD with an isolation window of 0.8 m/z with 0.3 m/z offset. With a mass tolerance of 10 ppm, precursors were excluded from further fragmentation for 45 s after single occurrences. The proteomics data were analyzed using a Proteome Discoverer Software version 2.3 (Thermo). Spectra were searched based on a SpCas9 database (FASTA Q99ZW2) using Sequest HT. The mass tolerance for the precursor ions was 10 ppm, and the mass tolerance for the fragment ions was 0.02 Da. Up to 2 missed cleavages were allowed, and variable oxidation on methionine residues was set. The probe modification was allowed at all residues (mass increase for ¹³C probe: 706.286 Da, mass increase for ¹²C probe: 704.279 Da). Peptide assignment was validated with Target Decoy PSM Validator. Spectra with high confidence were manually examined for isotopic coding and fragment matching. The data from three independent experiments are compiled and presented.

### Validation of Target Engagement in Live Cells.

HEK293T cells were plated in a 6-well plate (400,000 cells/well). The next day, cells were transfected with 2 pg of Cas9 expression plasmid (pX330, Addgene #42230)⁵¹ using Lipofectamine 3000 (Invitrogen). Eight hours after transfection, cells were split into 4 wells of a 12-well plate. Total 24 h after transfection, cells were treated with DMSO, Diazirine-BRD7586 (20 μM), or Diazirine-BRD7586 with BRD7586 (both at 20 μM) for 2 h. Cells were washed with PBS once, and 500 μL of fresh PBS was added to each well. The plate was placed on ice, and cells were irradiated with UV (365 nm) for 15 min. After the removal of PBS, cells were stored at−80° C. until further analysis. Thawed cells were suspended in a lysis buffer (25 mM HEPES, 50 mM KCl, 1% Triton X-100, 1× protease inhibitor cocktail, pH 7.4, 200 μL per well), and a brief sonication was performed to ensure cell lysis. Next, click chemistry was performed with 100 μM of biotin-azide, 350 μM of Cu-TBTA, and 1.5 mM of ascorbate. The reaction was proceeded for 2 h at room temperature with mild rotation, then proteins were precipitated by the addition of cold methanol (5-fold volume of the reaction mixture) to the mixture and keeping at −80° C. for >2 h. Protein pellet was obtained by centrifugation for 10 min at 16,000 g and 4° C. The pellet was washed with cold PBS:methanol (1:5 v/v), air-dried for 10 min, and resuspended in 100 μL of 1.2% SDS solution in PBS. Heating at 37° C. was required for complete solubilization of the pellet. Ten μL of the solution was saved for future analysis as an input. The rest 90 μL was diluted with PBS, and incubated with 40 μL of Streptavidin Magnetic Beads (Thermo #88816) in a final volume of 720 μL. The mixture was incubated for several hours at room temperature with mild rotation. Then, the beads were washed four times with 0.2% SDS solution in PBS (600 μL each). Finally, proteins were eluted from the bead by heating in an SDS-PAGE buffer. Immunoblotting was performed using mouse anti-SpCas9 (Abcam #191468, 1:1,000 dilution) and anti-mouse HRP (CST #7076, 1:5,000 dilution).

### Chemical Synthesis and Characterizations.

Synthetic procedures and characterizations are described herein.

### Statistical Analyses.

Two-tailed and unpaired t-tests were performed using Microsoft Excel to compare the means of two samples, and p values from the tests are presented in the figure legends.

### Reproducibility.

Independent experiments reported here were performed by different researchers using independently prepared biochemical reagents, or independent splits of the mammalian cell types were used.

### Data Availability.

Data generated in this study are provided herein and are available from the corresponding author upon reasonable request. Plasmids from Addgene (#43861 [www.addgene.org/43861], #47511 [www.addgene.org/47511], #42230 [www.addgene.org/42230]) were used in this study. Structural information from PDB (ID: 5F9R [www.rcsb.org/structure/5F9R]) was used in this study. High-throughput sequencing data have been deposited in the NCBI Sequence Read Archive database under accession #NNNNNNN.

### General Methods and Materials

All reactions containing water or air sensitive reagents were performed in oven-dried glassware under nitrogen or argon. All reagents were purchased and used as received from commercial sources without any further purification. Reactions were performed in round-bottom flasks or vials stirred with Teflon®-coated magnetic stir bars. Moisture and air-sensitive reactions were performed under a dry nitrogen/argon atmosphere. Moisture and air-sensitive liquids or solutions were transferred via nitrogen-flushed syringes. As necessary, organic solvents were degassed by bubbling nitrogen/argon through the liquid. The reaction progress was monitored by thin-layer chromatography (TLC) and ultra-performance liquid chromatography mass spectrometry (UPLC-MS). Flash column chromatography was performed using silica gel (60 Å mesh, 20-40 μm) on a Teledyne ISCO CombiFlash Rf system. Analytical TLC was performed using Merck Silica gel 60 F254 pre-coated plates (0.25 mm); illumination at 254 nm allowed the visualization of UV-active material. UPLC-MS was performed on a Waters ACQUITY UPLC I-Class PLUS System with an ACQUITY SQ Detector 2. Nuclear magnetic resonance (NMR) spectra were recorded on a Bruker 400 Spectrometer (¹H NMR, 400 MHz; ¹³C, 101 MHz) at the Broad Institute of MIT and Harvard. ¹H and ¹³C chemical shifts are indicated in parts per million (ppm) relative to SiMe₄ (6=0.00 ppm) and internally referenced to residual solvent signals. NMR solvents were purchased from Cambridge Isotope Laboratories, Inc., and NMR data were obtained in DMSO-d₆. Data for ¹H NMR are reported as follows: chemical shift value in ppm, multiplicity (s=singlet, d=doublet, t=triplet, dd=doublet of doublets, and m=multiplet), integration value, and coupling constant value in Hz. High-resolution mass spectra were recorded on a Thermo Q Exactive Plus mass spectrometer system equipped with an HESI-II electrospray ionization source at Harvard Center for Mass Spectrometry at the Harvard FAS Division of Science Core Facility.

### Synthesis of BRD7586

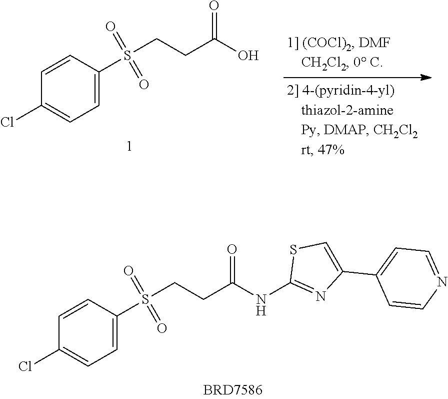 [SMILES: O=C(O)CCS(=O)(=O)c1ccc(Cl)cc1 ; O=C(CCS(=O)(=O)c1ccc(Cl)cc1)Nc1nc(-c2ccncc2)cs1]

In a 50 mL RBF, oxalyl chloride (0.183 g, 0.981 mmol) was added to solution of 3-((4-methoxyphenyl)sulfonyl)propanoic acid (0.3 g, 1.206 mmol) CH₂Cl₂ (12 mL) at 0° C. followed by catalytic amount of dry DMF (3 drops). The reaction was stirred at 0° C. for 2 h. The solvent was concentrated under reduced pressure, dried under vacuum for 2 h and used in the next step without any further purification. A solution of acid chloride in CH₂Cl₂ (6 mL) was added to a mixture of 4-Pyridin-4-yl-thiazol-2-ylamine (0.235 g, 1.326 mmol), Pyridine (0.477 g, 6.03 mmol) and DMAP (30 mg) in CH₂Cl₂ (12 mL) at 0° C. The reaction mixture was slowly warmed to rt and stirred at the same temperature for 12 h. The solvent was evaporated under reduced pressure. The residue was purified by using ISCO 12 g gold column. The column ran with CH₂Cl₂ grading to 5% MeOH—CH₂Cl₂ over 15 min then finally with 10% MeOH—CH₂Cl₂. 0.086 g of BRD7586 was isolated as an off white solid in 47% yield. ¹H NMR (400 MHz, DMSO-d₆) δ 12.45 (s, 1H), 8.76-8.53 (m, 2H), 7.99 (s, 1H), 7.92 (d, J=8.6 Hz, 1H), 7.86-7.79 (m, 2H), 7.72 (d, J=8.6 Hz, 2H), 3.71 (t, J=7.1 Hz, 2H), 2.84 (t, J=7.2 Hz, 2H). ¹³C NMR (101 MHz, DMSO-d₆) δ 168.6, 158.5, 150.7, 146.8, 141.3, 139.7, 137.6, 130.4, 130.1, 120.4, 112.9, 50.7, 29.0; HRMS (m/z): [M+H]⁺ calculated for Cl₇H₁₄ClN₃O₃S₂, 408.0243; found, 408.0238.

### Synthesis of BRD0033

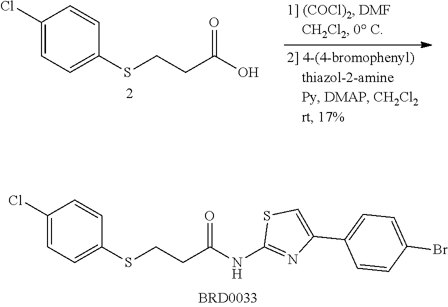

In a 50 mL RBF, oxalyl chloride (0.070 g, 0.553 mmol) was added to solution of 3-((4-chlorophenyl)sulfanyl)propanoic acid (0.1 g, 0.461 mmol) CH₂Cl₂ (10 mL) at 0° C. followed by catalytic amount of dry DMF (3 drops). The reaction was stirred at 0° C. for 2 h. The solvent was concentrated under reduced pressure, dried under vacuum for 2 h and used in the next step without any further purification. A solution of acid chloride in CH₂Cl₂ (6 mL) was added to a mixture of 2-amino-4-(4-bromophenyl)thiazole (0.117 g, 0.461 mmol), Pyridine (0.182 g, 2.31 mmol) and DMAP (5 mg) in CH₂Cl₂ (12 mL) at 0° C. The reaction mixture was slowly warmed to rt and stirred at the same temperature for 12 h. The solvent was evaporated under reduced pressure. The residue was purified by using ISCO 12 g gold column. The column ran with CH₂Cl₂ grading to 5% MeOH—CH₂Cl₂ over 15 min then finally with 10% MeOH—CH₂Cl₂. 36 mg of BRD0033 was isolated as an off white solid in 17% yield. ¹H NMR (400 MHz, DMSO-d₆) δ 12.30 (s, 1H), 7.83 (d, J=8.5 Hz, 1H), 7.69 (s, 1H), 7.62 (d, J=8.5 Hz, 2H), 7.39 (d, J=0.8 Hz, 4H), 3.27 (t, J=7.0 Hz, 2H), 2.80 (t, J=7.0 Hz, 2H). ¹³C NMR (101 MHz, DMSO-d₆) δ 169.5, 157.9, 147.6, 134.6, 133.5, 131.6, 130.5 130.0, 129.0, 127.6, 120.8, 108.9, 34.7, 27.6; HRMS (m/z): [M+H]⁺ calculated for C₁₈H₁₄BrClN₂OS₂, 452.9498; found, 452.9468.

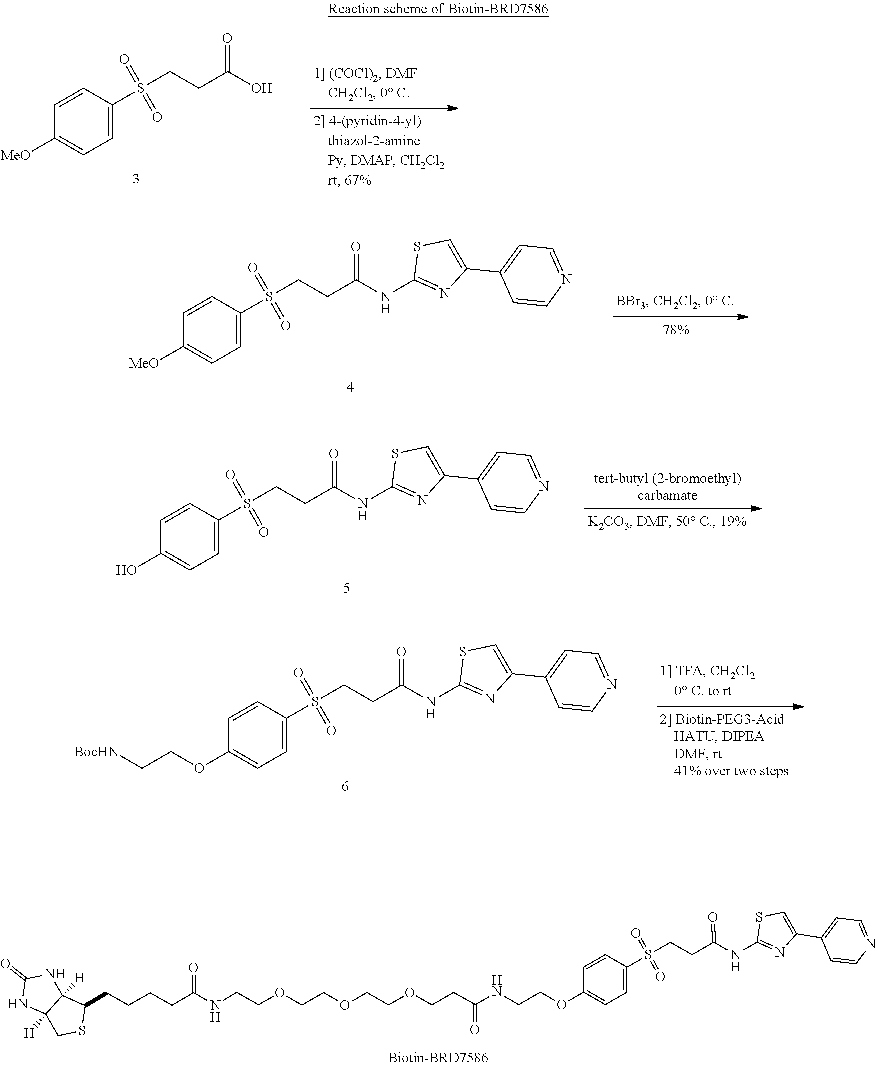 [SMILES: COc1ccc(S(=O)(=O)CCC(=O)O)cc1 ; COc1ccc(S(=O)(=O)CCC(=O)Nc2nc(-c3ccncc3)cs2)cc1 ; O=C(CCS(=O)(=O)c1ccc(O)cc1)Nc1nc(-c2ccncc2)cs1 ; CC(C)(C)OC(=O)NCCOc1ccc(S(=O)(=O)CCC(=O)Nc2nc(-c3ccncc3)cs2)cc1 ; O=C(CCCC[C@H]1SC[C@H]2NC(=O)N[C@H]21)NCCOCCOCCOCCC(=O)NCCOc1ccc(S(=O)(=O)CCC(=O)Nc2nc(-c3ccncc3)cs2)cc1]

### Synthesis of 3-((4-methoxyphenyl)sulfonyl)-N-(4-(pyridin-4-yl) thiazol-2-yl)propanamide (4)

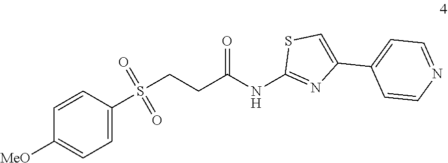 [SMILES: COc1ccc(S(=O)(=O)CCC(=O)Nc2nc(-c3ccncc3)cs2)cc1]

In a 50 mL RBF, oxalyl chloride (0.124 g, 0.981 mmol) was added to solution of 3-((4-methoxyphenyl)sulfonyl)propanoic acid 3 (0.2 g, 0.818 mmol) CH₂Cl₂ (10 mL) at 0° C. followed by catalytic amount of dry DMF (3 drops). The reaction was stirred at 0° C. for 2 h. The solvent was concentrated under reduced pressure, dried under vacuum for 2 h and used in the next step without any further purification. A solution of acid chloride in CH₂Cl₂ (6 mL) was added to a mixture of 4-Pyridin-4-yl-thiazol-2-ylamine (0.16 g, 0.899 mmol), Pyridine (0.323 g, 4.09 mmol) and DMAP (25 mg) in CH₂Cl₂ (12 mL) at 0° C. The reaction mixture was slowly warmed to rt and stirred at the same temperature for 12 h. The solvent was evaporated under reduced pressure. The residue was purified by using ISCO 12 g gold column. The column ran with CH₂Cl₂ grading to 5% MeOH—CH₂Cl₂ over 15 min then finally with 10% MeOH—CH₂Cl₂. 0.223 g of 3-((4-methoxyphenyl)sulfonyl)-N-(4-(pyridin-4-yl) thiazol-2-yl)propanamide (4) was isolated as an off white solid in 67% yield. ¹H NMR (400 MHz, DMSO-d₆) δ 12.41 (s, 1H), 8.62 (d, J=6.1 Hz, 1H), 7.97 (s, 1H), 7.88-7.76 (m, 4H), 7.13 (d, J=8.9 Hz, 2H), 3.60 (t, J=7.1 Hz, 2H), 2.83 (t, J=7.1 Hz, 2H). ¹³C NMR (101 MHz, DMSO-d₆) δ 168.3, 163.4, 158.2, 150.3, 146.3, 140.9, 130.2, 129.8, 119.9, 114.6, 112.4, 55.7, 50.6, 28.7; HRMS (m/z): [M+H]⁺ calculated for C₁₈H₁₇N₃O₄S₂, 404.0739; found, 404.0732.

### Synthesis of 3-((4-hydroxyphenyl)sulfonyl)-N-(4-(pyridin-4-yl)thiazol-2-yl)propanamide (5)

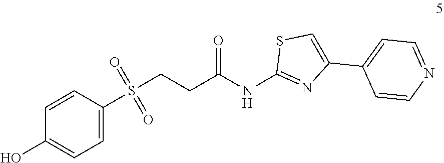 [SMILES: O=C(CCS(=O)(=O)c1ccc(O)cc1)Nc1nc(-c2ccncc2)cs1]

In a 100 mL RBF, BBr₃ (5.32 mL, 5.32 mmol, 1.0 M solution in CH₂Cl₂) was added to solution of amide 3 (0.265 g, 0.656 mmol) CH₂Cl₂ (30 mL) at 0° C. The reaction was stirred at the same temperature for 5-6 h. The reaction was quenched with MeOH (4 mL) at 0° C. and the solvent was evaporated under reduced pressure. The residue was purified by using ISCO 12 g gold column. The column ran with CH₂Cl₂ grading to 5% MeOH—CH₂Cl₂ over 15 min then finally grading to 15% MeOH—CH₂Cl₂ (1% NH₄OH). 200 mg of 3-((4-hydroxyphenyl)sulfonyl)-N-(4-(pyridin-4-yl)thiazol-2-yl)propanamide (5) was isolated as an off white solid in 78% yield. ¹H NMR (400 MHz, DMSO-d₆) δ 12.41 (s, 1H), 10.61 (s, 1H), 8.61 (d, J=6.1 Hz, 1H), 7.97 (s, 1H), 7.88-7.78 (m, 2H), 7.71 (d, J=8.8 Hz, 2H), 6.95 (d, J=8.8 Hz, 1H), 3.55 (t, J=7.2 Hz, 2H), 2.82 (t, J=7.3 Hz, 2H). ¹³C NMR (101 MHz, DMSO-d₆) δ 168.3, 162.3, 158.2, 150.3, 146.3, 140.9, 130.3, 128.1, 119.9, 115.8, 112.3, 50.7, 28.7; HRMS (m/z): [M+H]⁺ calculated for C₁₇H₁₅N₃O₄S₂, 390.0582; found, 390.0577.

### Synthesis of tert-butyl (2-(4-((3-oxo-3-((4-(pyridin-4-yl)thiazol-2-yl)amino)propyl)sulfonyl)phenoxy)ethyl)carbamate (6)

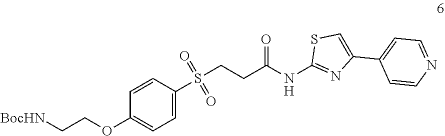 [SMILES: CC(C)(C)OC(=O)NCCOc1ccc(S(=O)(=O)CCC(=O)Nc2nc(-c3ccncc3)cs2)cc1]

In a 10 mL RBF, a mixture of 3-((4-hydroxyphenyl)sulfonyl)-N-(4-(pyridin-4-yl)thiazol-2-yl)propanamide (5) (0.054 g, 0.138 mmol), Cs₂CO₃ (0.068 g, 0.208 mmol), tert-butyl (2-bromoethyl) carbamate (0.034 g, 0.152 mmol) in DMF (1 mL) was stirred at 50° C. for 22 h. The solvent was evaporated under reduced pressure. The residue was purified by using ISCO 4 g gold column. The column ran with CH₂Cl₂ grading to 5% MeOH—CH₂Cl₂ (1% NH₄OH) over 15 min. 10 mg of tert-butyl (2-(4-((3-oxo-3-((4-(pyridin-4-yl)thiazol-2-yl)amino)propyl)sulfonyl)phenoxy)ethyl)carbamate (6) was isolated as an off white solid in 19% yield. ¹H NMR (400 MHz, DMSO-d₆) δ 8.65-8.60 (m, 2H), 7.95 (s, 1H), 7.85-7.78 (m, 4H), 7.14 (d, J=8.9 Hz, 2H), 7.02 (t, J=5.8 Hz, 1H), 4.03 (t, J=5.8 Hz, 2H), 3.60 (t, J=7.2 Hz, 2H), 3.29 (d, J=5.7 Hz, 1H), 2.83 (t, J=7.2 Hz, 2H), 1.38 (s, 9H).¹³C NMR (101 MHz, DMSO-d₆) δ 168.4, 162.6, 158.5, 155.7, 150.2, 146.3, 140.9, 130.2, 129.9, 119.9, 115.0, 112.2, 77.8, 67.0, 50.7, 48.6, 28.8, 28.2; HRMS (m/z): [M+H]⁺ calculated for C₂₄H₂₈N₄O₆S₂, 533.1529; found, 533.1526.

### Synthesis of Biotin-BRD7586

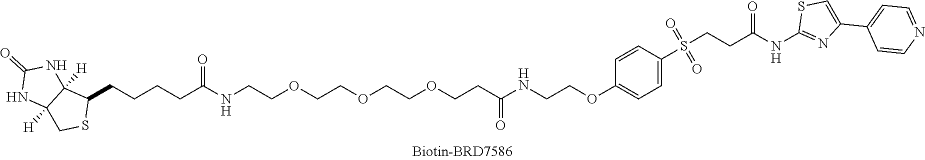

In a 7 mL vial, TFA (0.2 mL) was added to solution of tert-butyl (2-(4-((3-oxo-3-((4-(pyridin-4-yl)thiazol-2-yl)amino)propyl)sulfonyl)phenoxy)ethyl)carbamate (6) (9 mg, 0.0168 mmol) CH₂Cl₂ (1 mL) at 0° C. The reaction was stirred at 0° C. for 2 h. The solvent was concentrated under reduced pressure and dried under vacuum for 2 h. The resulting crude amine was used in the next step without any further purification. DIPEA (0.0065 g, 0.0504 mmol) was added to solution of crude amine, Biotin-PEG3-Acid (0.0075 g, 0.0168 mmol) and HATU (0.0076 g, 0.0202 mmol), DMF (1 mL) at 0° C. The reaction was slowly warmed to rt and stirred at the same temperature for 18 h. The solvent was evaporated under reduced pressure. The residue was diluted with CH₂Cl₂ (20 mL) and washed with Sat. NaHCO₃ solution (4 mL). The org. layer was dried over anhydrous Na₂SO₄, filtered and concentrated under reduced pressure. The residue was purified by using ISCO 12 g gold column. The column ran with CH₂Cl₂ grading to 5% MeOH—CH₂Cl₂ over 10 min then finally grading to 20% MeOH—CH₂Cl₂ (1% NH₄OH). 6 mg of Biotin-BRD7586 was isolated as an off white solid in 41% yield over two steps. ¹H NMR (400 MHz, DMSO-d₆) δ 8.68-8.59 (m, 2H), 8.12 (t, J=5.5 Hz, 1H), 7.97 (s, OH), 7.88-7.78 (m, 3H), 7.15 (d, J=8.9 Hz, 1H), 6.40 (s, OH), 6.34 (s, OH), 4.31 (dd, J=7.7, 5.1 Hz, 1H), 4.18-4.06 (m, 1H), 4.04 (t, J=5.6 Hz, 1H), 3.61 (dt, J=8.0, 4.4 Hz, 2H), 3.48 (d, J=4.5 Hz, 4H), 3.40 (dt, J=9.4, 5.7 Hz, 2H), 3.19 (dd, J=7.1, 5.3 Hz, 2H), 3.09 (ddd, J=8.6, 6.1, 4.4 Hz, 1H), 2.83 (dt, J=10.6, 6.1 Hz, 1H), 2.58 (d, J=12.4 Hz, 1H), 2.34 (t, J=6.4 Hz, 1H), 2.07 (t, J=7.4 Hz, 1H), 1.74-1.41 (m, 2H), 1.30 (td, J=18.4, 16.4, 9.0 Hz, 1H). ¹³C NMR (101 MHz, DMSO-d₆) δ 172.1, 170.5, 168.2, 162.7, 162.6, 158.1, 150.2, 146.3, 140.8, 130.2, 130.0, 119.9, 115.1, 112.3, 69.7, 69.6, 69.5, 69.5, 69.1, 66.9, 66.7, 61.0, 59.2, 55.4, 50.6, 48.6, 38.4, 37.9, 36.0, 35.1, 28.7, 28.2, 28.0, 25.2; HRMS (m/z): [M+2H]²⁺ calculated for C₃₈H₅₁N₇O₁₀S₃, 431.6508; found, 431.6502.

### Synthesis of BRD7586-diazirine

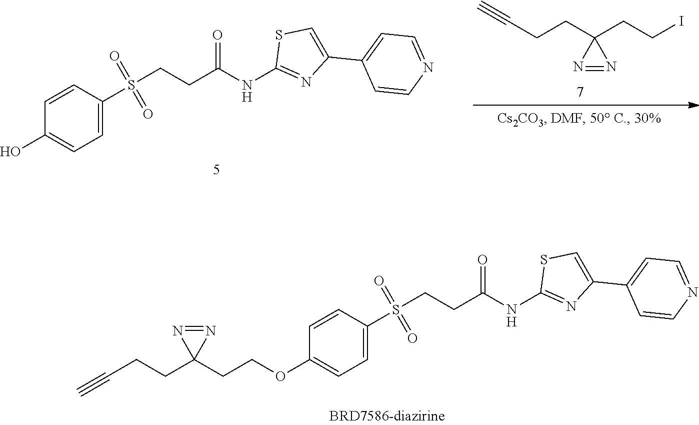

In a 10 mL RBF, a mixture of 3-((4-hydroxyphenyl)sulfonyl)-N-(4-(pyridin-4-yl)thiazol-2-yl)propanamide (5) (0.0229 g, 0.058 mmol), Cs₂CO₃ (0.0287 g, 0.088 mmol), Diazirine Iodide 7 (0.0158 g, 0.064 mmol) in DMF (1 mL) was stirred at 50° C. for 22 h. The solvent was evaporated under reduced pressure. The residue was purified by using ISCO 4 g gold column. The column ran with CH₂Cl₂ grading to 5% MeOH—CH₂Cl₂ over 15 min then finally grading to 10% MeOH—CH₂Cl₂ (1% NH₄OH). 9 mg of BRD7586-diazirine was isolated as an off white solid in 30% yield. ¹H NMR (400 MHz, DMSO-d₆) δ 12.38 (s, 1H), 8.66-8.57 (m, 2H), 7.96 (s, 1H), 7.88-7.76 (m, 4H), 7.12 (d, J=8.7 Hz, 2H), 3.87 (t, J=6.1 Hz, 2H), 3.60 (t, J=7.1 Hz, 2H), 3.17 (s, 1H), 2.89-2.76 (m, 3H), 2.07 (s, 1H), 2.02 (td, J=7.4, 2.7 Hz, 2H), 1.86 (t, J=6.1 Hz, 2H), 1.64 (t, J=7.3 Hz, 2H). ¹³C NMR (101 MHz, DMSO-d₆) δ 168.2, 162.3 158.1, 150.1, 146.3, 141.0, 130.2, 130.1, 119.9, 115.0, 112.4, 83.1, 71.7, 63.1, 50.6, 48.6, 31.6, 28.7, 26.8, 12.6; HRMS (m/z): [M+H]⁺ calculated for C₂₄H₂₃N₅O₄S₂, 510.1270; found, 510.1266.

### References for Example 2 Methods

49. Clement, K. et al. CRISPResso2 provides accurate and rapid genome editing sequence analysis. Nat. Biotech. 37, 224-226 (2019).
50. Di, L., Kerns, E. H., Hong, Y. & Chen, H. Development and application of high throughput plasma stability assay for drug discovery. Int. J. Pharm. 297, 110-119 (2005).
51. Cong, L. et al. Multiplex Genome Engineering Using CRISPR/Cas Systems. Science 339, 819-823 (2013).

TABLE 7
Primary Screen Library Information used for identifying SpCas9 inhibitors.

| | | | Number of positive | | Number of | Number of positive |
|---|---|---|---|---|---|---|
| Compound | | Number of | compounds in | | cherrypicked | compounds in |
| group | Library^{a} | compounds | primary screen | | compounds^{b} | secondary screen |
| Commercial | ChemDiv 6 | 44,000 | 93 | (0.21% hit rate) | 83 | 0 |
| compounds | ChemDiv 7 | 49,128 | 382 | (0.77% hit rate) | 374 | 8 |
| | Enamine 2 | 26,929 | 90 | (0.33% hit rate) | 79 | 8 |
| Known | NIH Clinical | 450 | 4 | (0.89% hit rate) | 4 | 0 |
| bioactive | Collection 1-2014 | | | | | |
| compounds | Selleck Bioactive | 1,902 | 10 | (0.53% hit rate) | 7 | 0 |
| | Compound Library | | | | | |

^{a}Compound plates were used as provided from the ICCB-Longwood Screening Facility. Thus, the test concentration depends on the type of library. Most compounds were tested at 10 μM or 5 μg/mL (which corresponds to 10 μM assuming the molecular weight of 500).
^{b}PAINS-flagged compounds were removed from the hit list.

TABLE 8
DNAs used for fluorescence polarization assay to detect
Cas nuclease-DNA interactions.

| DNA name | Sequences |
|---|---|
| SaCas9 0-PAM, 3′ FAM | GTGTCCGAACGGAACGGTATCGATACGTCGCTGTTAGCTACTAA |
| (Top strand) | TTGCACCAGCAGCGCCCTATGGAC/6-FAM/ (SEQ ID NO: 3) |
| SaCas9 0-PAM, 3′ FAM | GTCCATAGGGCGCTGCTGGTGCAATTAGTAGCTAACAGCGACGT |
| (Bottom strand) | ATCGATACCGTTCCGTTCGGACAC (SEQ ID NO: 4) |
| SaCas9 12-PAM, 3′ FAM | GTGTCGGATCGGATGGGTATCCATTCGACCCTGTAAGGGTCTAA |
| (Top strand) | TTCCAGGATCAGCCGGGTATCCAC/6-FAM/ (SEQ ID NO: 5) |
| SaCas9 12-PAM, 3′ FAM | GTGGATACCCGGCTGATCCTGGAATTAGACCCTTACAGGGTCGA |
| (Bottom strand) | ATGGATACCCATCCGATCCGACAC (SEQ ID NO: 6) |
| SaCas9 12-PAM | GTGTCGGATCGGATGGGTATCCATTCGACCCTGTAAGGGTCTAA |
| Unlabeled | TTCCAGGATCAGCCGGGTATCCAC (SEQ ID NO: 7) |
| (Top strand) | |
| SaCas9 12-PAM | GTGGATACCCGGCTGATCCTGGAATTAGACCCTTACAGGGTCGA |
| Unlabeled | ATGGATACCCATCCGATCCGACAC (SEQ ID NO: 8) |
| (Bottom strand) | |
| FnCas12a 0-PAM, 3′ FAM | CTATCGCTATCCATAGGCATATATAGCCCATACTATGCCTATAGC |
| (Top strand) | TATGATAGGGATAGATAC/6-FAM/ (SEQ ID NO: 9) |
| FnCas12a 0-PAM, 3′ FAM | GTATCTATCCCTATCATAGCTATAGGCATAGTATGGGCTATATAT |
| (Bottom strand) | GCCTATGGATAGCGATAG (SEQ ID NO: 10) |
| FnCas12a 12-PAM, 3′ | CTTTCGCTTTCCAAAGGCATTTAAAGCCCAAACTTTGCCTAAAGC |
| FAM | TTTGAAAGGGAAAGAAAC/6-FAM/ (SEQ ID NO: 11) |
| (Top strand) | |
| FnCas12a 12-PAM, 3′ | GTTTCTTTCCCTTTCAAAGCTTTAGGCAAAGTTTGGGCTTTAAAT |
| FAM | GCCTTTGGAAAGCGAAAG (SEQ ID NO: 12) |
| (Bottom strand) | |
| FnCas12a 0-PAM, 5′ FAM | /6- |
| (Top strand) | FAM/CTATCGCTATCCATAGGCATATATAGCCCATACTATGCCTA |
| | TAGCTATGATAGGGATAGATAC (SEQ ID NO: 13) |
| FnCas12a 0-PAM, 5′ FAM | GTATCTATCCCTATCATAGCTATAGGCATAGTATGGGCTATATAT |
| (Bottom strand) | GCCTATGGATAGCGATAG (SEQ ID NO: 14) |
| FnCas12a 12-PAM, 5′ | /6- |
| FAM | FAM/CTTTCGCTTTCCAAAGGCATTTAAAGCCCAAACTTTGCCTA |
| (Top strand) | AAGCTTTGAAAGGGAAAGAAAC (SEQ ID NO: 15) |
| FnCas12a 12-PAM, 5′ | GTTTCTTTCCCTTTCAAAGCTTTAGGCAAAGTTTGGGCTTTAAAT |
| FAM | GCCTTTGGAAAGCGAAAG (SEQ ID NO: 16) |
| (Bottom strand) | |
| FnCas12a 12-PAM | CTTTCGCTTTCCAAAGGCATTTAAAGCCCAAACTTTGCCTAAAGC |
| Unlabeled | TTTGAAAGGGAAAGAAAC (SEQ ID NO: 17) |
| (Top strand) | |
| FnCas12a 12-PAM | GTTTCTTTCCCTTTCAAAGCTTTAGGCAAAGTTTGGGCTTTAAAT |
| Unlabeled | GCCTTTGGAAAGCGAAAG (SEQ ID NO: 18) |
| (Bottom strand) | |

TABLE 9
DNAs used for cumulative activity assays.

| DNA name | Sequences |
|---|---|
| SpCas9 substrate | /Alex647N/TAATACGACTCACTATAGGACG |
| forward | CGACCGAAATGGTGAAGGACGGGT |
| | (SEQ ID NO: 19) |
| SpCas9 substrate | ACCCGTCCTTCAGGTTTTCGGTCGCGTCCTAT |
| reverse | AGTGAGTCGTATTA |
| | (SEQ ID NO: 20) |
| SpCas9 displacer | ATAGTGAGTCGTATTA/IAbRQSp/ |
| | (SEQ ID NO: 21) |
| SaCas9 substrate | /Alex647N/ACTCACTATAGGGACGCGACCG |
| forward | AAATGGTGAAGGACGGGTCCAGTGCTTCGG |
| | (SEQ ID NO: 22) |
| SaCas9 substrate | CCGAAGCACTGGACCCGTCCTTCACCATTTCG |
| reverse | GTCGCGTCCCTATAGTGAGT |
| | (SEQ ID NO: 23) |
| SaCas9 displacer | CGTCCCTATAGTGAGT/IAbRQSp/ |
| | (SEQ ID NO: 24) |
| FnCas12a NTS | CGTCCTTCACCATTTCGGTCGCGTCCCTATAG |
| substrate forward | TGAGTCGTATTAGTTCCAT/AlexF647N/ |
| | (SEQ ID NO: 25) |
| FnCas12a NTS | ATGGAACTAATACGACTCACTATAGGGACGCG |
| substrate reverse | ACCGAAATGGTGAAGGACG |
| | (SEQ ID NO: 26) |
| FnCas12a NTS | /IABKFQ/ATGGAACTAATACGAC |
| displacer | (SEQ ID NO: 27) |
| FnCas12a TS | /AlexF647N/ATGGAACTAATACGACTCACT |
| substrate forward | ATAGGGACGCGACCGAAATGGTGAAGGACG |
| | (SEQ ID NO: 28) |
| FnCas12a TS | CGTCCTTCACCATTTCGGTCGCGTCCCTATAG |
| substrate reverse | TGAGTCGTATTAGTTCCAT |
| | (SEQ ID NO: 29) |
| FnCas12a TS | GTCGTATTAGTTCCAT/IABKFQ/ |
| displacer | (SEQ ID NO: 30) |

TABLE 10
Primers for gRNA synthesis.

| Primer name | Primer sequence |
|---|---|
| SpCas9 | AAAAGCACCGACTCGGTGCCACTTTTTCAAGT |
| Universal reverse | TGATAACGGACTAGCCTTATTTTAACTTGCTA |
| | TTTCTAGCTCTAAAAC |
| | (SEQ ID NO: 31) |
| SpCas9 | TAATACGACTCACTATAGCTATAGGACGCGAC |
| Spinach forward | CGAAAGTTTTAGAGCTAGAAAT |
| | (SEQ ID NO: 32) |
| SpCas9 | TAATACGACTCACTATAGGGCACGGGCAGCTT |
| eGFP forward | GCCGGGTTTTAGAGCTAGAAAT |
| | (SEQ ID NO: 33) |
| SpCas9 | TAATACGACTCACTATAGGTCCAGGGGTCTTA |
| GAPDH forward | CTCCTGTTTTAGAGCTAGAAAT |
| | (SEQ ID NO: 34) |
| SaCas9 | AAAATCTCGCCAACAAGTTGACGAGATAAACA |
| Universal reverse | CGGCATTTTGCCTTGTTTTAGTAGATTCTGTT |
| | TCCAGAG |
| | (SEQ ID NO: 35) |
| SaCas9 | TAATACGACTCACTATAGGGACGCGACCGAAA |
| Spinach forward | TGGTGAAGGGTTTTAGTACTCTGGAA |
| | (SEQ ID NO: 36) |
| FnCas12a | GAAATTAATACGACTCACTATAGGG |
| Universal reverse | (SEQ ID NO: 37) |
| FnCas12a | ACGACTCACTATAGGGACGCGACCATCTACAA |
| Spinach forward | CAGTAGAAATTACCCTATAGTGAGTCGTATTA |
| | ATTTC |
| | (SEQ ID NO: 38) |

TABLE 11
On-target and off-target spacer sequences used in
plasmid-based Cas9 and gRNA delivery. Mismatches
in the off-target sequences are shown in red.

| Target | Spacer sequence |
|---|---|
| eGFP On-target | GGGCACGGGCAGCTTGCCGG |
| | (SEQ ID NO: 39) |
| GAPDH On-target | GGTCCAGGGGTCTTACTCCT |
| | (SEQ ID NO: 40) |
| EMX1 On-target | GAGTCCGAGCAGAAGAAGAA |
| | (SEQ ID NO: 41) |
| EMX1 Off-target | GAGTCTAAGCAGAAGAAGAA |
| | (SEQ ID NO: 42) |
| FANCF On-target | GGAATCCCTTCTGCAGCACC |
| | (SEQ ID NO: 43) |
| FANCF Off-target | GGAACCCCGTCTGCAGCACC |
| | (SEQ ID NO: 44) |
| VEGFA On-target | GGGTGGGGGGAGTTTGCTCC |
| | (SEQ ID NO: 45) |
| VEGFA Off-target | CGGGGGAGGGAGTTTGCTCC |
| | (SEQ ID NO: 46) |

TABLE 12
ssODN sequence used for HiBiT knock-in assay.

| ssODN name | ssODN sequence |
|---|---|
| GAPDH_HiBiT | TCTTCTAGGTATGACAACGAATTTGGCTACAGCAAC |
| | AGGGTGGTGGACCTCATGGCCCACATGGCCTCCAAG |
| | GAGGTGAGCGGCTGGCGGCTGTTCAAGAAGATTAGC |
| | TAAGACCCCTGGACCACCAGCCCCAGCAAGAGCACA |
| | AGAGGAAGAGAGAGACCCTCACTGCTGGGGAGTCCC |
| | TGC |
| | (SEQ ID NO: 47) |

TABLE 13
Primers used for target amplifications
in targeted deep sequencing.

| Primer name | Primer sequence |
|---|---|
| eGFP On-target | ACACTCTTTCCCTACACGACGCTCTTCCGATCT |
| forward | NNNNACGTAAACGGCCACAAGTTC |
| | (SEQ ID NO: 48) |
| eGFP On-target | TGGAGTTCAGACGTGTGCTCTTCCGATCTGTCG |
| reverse | TCCTTGAAGAAGATGGTG |
| | (SEQ ID NO: 49) |
| EMX1 On-target | ACACTCTTTCCCTACACGACGCTCTTCCGATCT |
| forward | NNNNCAGCTCAGCCTGAGTGTTGA |
| | (SEQ ID NO: 50) |
| EMX1 On-target | TGGAGTTCAGACGTGTGCTCTTCCGATCTCTCG |
| reverse | TGGGTTTGTGGTTGC |
| | (SEQ ID NO: 51) |
| EMX1 Off-target | ACACTCTTTCCCTACACGACGCTCTTCCGATCT |
| forward | NNNNCACGGCCTTTGCAAATAGAG |
| | (SEQ ID NO: 52) |
| EMX1 Off-target | TGGAGTTCAGACGTGTGCTCTTCCGATCTGGCT |
| reverse | TTCACAAGGATGCAGT |
| | (SEQ ID NO: 53) |
| FANCF On-target | ACACTCTTTCCCTACACGACGCTCTTCCGATCT |
| forward | NNNNCATTGCAGAGAGGCGTATCA |
| | (SEQ ID NO: 54) |
| FANCF On-target | TGGAGTTCAGACGTGTGCTCTTCCGATCTGGGG |
| reverse | TCCCAGGTGCTGAC |
| | (SEQ ID NO: 55) |
| FANCF Off-target | ACACTCTTTCCCTACACGACGCTCTTCCGATCT |
| forward | NNNNGCGGGCAGTGGCGTCTTAGTCG |
| | (SEQ ID NO: 56) |
| FANCF Off-target | TGGAGTTCAGACGTGTGCTCTTCCGATCTCCCT |
| reverse | GGGTTTGGTTGGCTGCTC |
| | (SEQ ID NO: 57) |
| VEGFA On-target | ACACTCTTTCCCTACACGACGCTCTTCCGATCT |
| forward | NNNNGGCTCTCTGTACATGAAGCAACT |
| | (SEQ ID NO: 58) |
| VEGFA On-target | TGGAGTTCAGACGTGTGCTCTTCCGATCTCCTA |
| reverse | GTGACTGCCGTCTGC |
| | (SEQ ID NO: 59) |
| VEGFA Off-target | ACACTCTTTCCCTACACGACGCTCTTCCGATCT |
| forward | NNNNCTCAGCACCTGCACTTCTTG |
| | (SEQ ID NO: 60) |
| VEGFA Off-target | TGGAGTTCAGACGTGTGCTCTTCCGATCTCAGA |
| reverse | TGTGGCCCTGAGAGAG |
| | (SEQ ID NO: 61) |

TABLE 14
List of peptides identified from three independent
chemoproteomics experiments.

| | | | | m/z | | XCorr |
|---|---|---|---|---|---|---|
| Exp # | Sequence | Modifications | Charge | theoretical | # PSM | score |
| 1 | EHPVENTQLQNEK | Probe (Q10) | 4 | 548.9904 | 1 | 1.26 |
| | (SEQ ID NO: 62) | | | | | |
| 1 | EHPVENTQLQNEK | Probe (H2) | 3 | 731.6515 | 1 | 3.32 |
| | (SEQ ID NO: 62) | | | | | |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of inhibiting activity of an RNA-guided endonuclease, the method comprising contacting the RNA-guided endonuclease with a compound of formula (I) 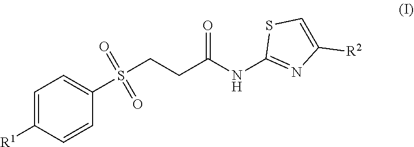
wherein R¹ and R² are independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof.

2. The method of claim 1, wherein the inhibitor is the compound of formula I and R¹ is H, F, Cl, OH, Me, or OMe and R² is 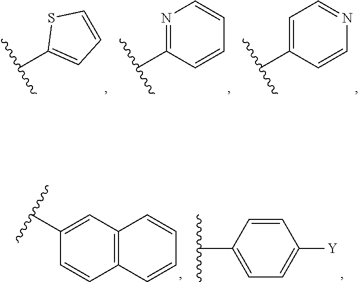
wherein Y is selected from substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen,
optionally wherein R⁵ is 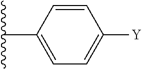
and wherein Y is selected from F, Cl, Br, I, OMe, or Et, and
optionally wherein the compound is

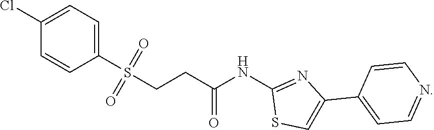 [SMILES: O=C(CCS(=O)(=O)c1ccc(Cl)cc1)Nc1nc(-c2ccncc2)cs1]

3. (canceled)

4. (canceled)

5. The method of claim 1, wherein the compound inhibits the activity of an RNA-guided endonuclease reversibly.

6. The method of claim 1, wherein the method is performed in vitro.

7. The method of claim 1, wherein the method is performed in vivo.

8. The method of claim 1, wherein the method is performed in a cell,
optionally wherein the cell is a germline cell, a eukaryotic cell, or a prokaryotic cell,
optionally wherein the prokaryotic cell is a bacterium,
optionally wherein the eukaryotic cell is a human cell, a mammalian cell, an insect cell, a plant cell, or a yeast cell.

9. (canceled)

10. (canceled)

11. (canceled)

12. (canceled)

13. (canceled)

14. The method of claim 8, wherein the cell is in an organism, optionally wherein the organism is a human, mammal, vertebrate, invertebrate, insect, or plant.

15. (canceled)

16. The method of claim 1, wherein the RNA-guided endonuclease is Cas9.

17. The method of claim 1, wherein the RNA-guided endonuclease is Streptococcus pyogenes Cas9 or a variant thereof.

18. The method of claim 1, wherein the RNA-guided endonuclease is Staphylococcus aureus Cas 9 (SaCas9).

19. A method of treating a subject, comprising:
a. administering an RNA-guided endonuclease-RNA complex or a reagent causing expression of the RNA-guided endonuclease-RNA complex to the subject; and
b. administering an effective amount of a compound as defined in any one of the preceding claims.

20. A RNA-guided endonuclease inhibitor comprising a compound of formula (I) 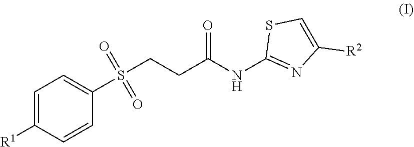
wherein R¹ and R² are independently selected from a hydrogen, alkane, alkene, alkyne, ether, alcohol, amine, nitrile, nitro, thiol, sulfone, sulfonate, halogen, carbonyl; acyl; ketone; carboxylate ester; amide; enone; acid anhydride; imide, aliphatic halide such as —OCF₂Cl; cyclic hydrocarbon, an unsaturated cyclic hydrocarbon, a heterocycle, one or more fused rings comprising any combination of any previously mentioned rings, or any combination of the groups previously mentioned thereof.

21. The composition of claim 20, wherein the inhibitor is the compound of formula I and R¹ is H, F, Cl, OH, Me, or OMe and R² is 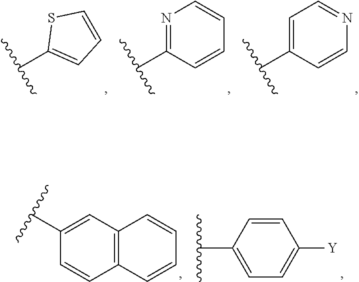
wherein Y is selected from substituted or unsubstituted alkyl, alkene, alkyne, halogen, alkoxy optionally substituted with one or more carbon-carbon double or triple bonds, or nitrile group, amino groups, amide, sulfonamide, cyano, hydroxy, mercapto, trifluoromethyl, alkoxy, alkylthio, thioalkoxy, arylalkyl, heteroaryl, alkylamino, dialkylamino, alkylsulfano, and keto groups, carboxylate, amide; and wherein the substituted alkyl is substituted with one or more substituents independently selected from the group comprising amino groups, amide, sulfonamide, halogen, cyano, carboxy, hydroxy, mercapto, trifluoromethyl, alkyl, alkoxy, alkylthio, thioalkoxy, arylalkyl, heteroaryl, alkylamino, dialkylamino, alkylsulfano, and keto, H, alkyl, heteroalkyl, aryl, heteroaryl, cycloalkyl, alkylene, alkyne, hydroxyl, carboxyl, carboxylate, amine and/or a halogen.

22. The composition of claim 21, wherein R⁵ is 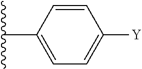
and wherein Y is selected from F, Cl, Br, I, OMe, or Et.

23. The composition of claim 21, wherein the compound is 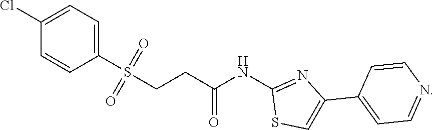 [SMILES: O=C(CCS(=O)(=O)c1ccc(Cl)cc1)Nc1nc(-c2ccncc2)cs1]


SEQUENCE LISTING (62 sequences)

SEQ ID NO: 1 - RNA, 106 nt, synthetic construct
  misc_feature 1..106: note = Synthetic
  source 1..106: mol_type = other RNA
gggagacgcaactgaatgaaatggtgaaggacgggtccaggtgtggctgcttcggcagtgcagcttgttgagtagagtgtgagctccgcgtaactagtcgcgtcac

SEQ ID NO: 2 - RNA, 100 nt, synthetic construct
  misc_feature 1..100: note = Synthetic
  source 1..100: mol_type = other RNA
gctataggacgcgaccgaaagttttagagctagaaatagcaagttaaaataaggctagtccgttatcaacttgaaaaagtggcaccgagtcggtgctttt

SEQ ID NO: 3 - DNA, 68 bp, Staphylococcus aureus
  misc_feature 68: note = 3' /6-FAM/ Fluorescein modification
  source 1..68: mol_type = other DNA
gtgtccgaacggaacggtatcgatacgtcgctgttagctactaattgcaccagcagcgccctatggac

SEQ ID NO: 4 - DNA, 68 bp, Staphylococcus aureus
  source 1..68: mol_type = other DNA
gtccatagggcgctgctggtgcaattagtagctaacagcgacgtatcgataccgttccgttcggacac

SEQ ID NO: 5 - DNA, 68 bp, Staphylococcus aureus
  misc_feature 68: note = 3' /6-FAM/ Fluorescein modification
  source 1..68: mol_type = other DNA
gtgtcggatcggatgggtatccattcgaccctgtaagggtctaattccaggatcagccgggtatccac

SEQ ID NO: 6 - DNA, 68 bp, Staphylococcus aureus
  source 1..68: mol_type = other DNA
gtggatacccggctgatcctggaattagacccttacagggtcgaatggatacccatccgatccgacac

SEQ ID NO: 7 - DNA, 68 bp, Staphylococcus aureus; identical to SEQ ID NO: 5
  source 1..68: mol_type = other DNA

SEQ ID NO: 8 - DNA, 68 bp, Staphylococcus aureus; identical to SEQ ID NO: 6
  source 1..68: mol_type = other DNA

SEQ ID NO: 9 - DNA, 63 bp, Francisella tularensis
  misc_feature 63: note = 3' /6-FAM/ Fluorescein modification
  source 1..63: mol_type = other DNA
ctatcgctatccataggcatatatagcccatactatgcctatagctatgatagggatagatac

SEQ ID NO: 10 - DNA, 63 bp, Francisella tularensis
  source 1..63: mol_type = other DNA
gtatctatccctatcatagctataggcatagtatgggctatatatgcctatggatagcgatag

SEQ ID NO: 11 - DNA, 63 bp, Francisella tularensis
  misc_feature 63: note = 3' /6-FAM/ Fluorescein modification
  source 1..63: mol_type = other DNA
ctttcgctttccaaaggcatttaaagcccaaactttgcctaaagctttgaaagggaaagaaac

SEQ ID NO: 12 - DNA, 63 bp, Francisella tularensis
  source 1..63: mol_type = other DNA
gtttctttccctttcaaagctttaggcaaagtttgggctttaaatgcctttggaaagcgaaag

SEQ ID NO: 13 - DNA, 63 bp, Francisella tularensis; identical to SEQ ID NO: 9
  misc_feature 1: note = 5' /6-FAM/ Fluorescein modification
  source 1..63: mol_type = other DNA

SEQ ID NO: 14 - DNA, 63 bp, Francisella tularensis; identical to SEQ ID NO: 10
  source 1..63: mol_type = other DNA

SEQ ID NO: 15 - DNA, 63 bp, Francisella tularensis; identical to SEQ ID NO: 11
  misc_feature 1: note = 5' /6-FAM/ Fluorescein modification
  source 1..63: mol_type = other DNA

SEQ ID NO: 16 - DNA, 63 bp, Francisella tularensis; identical to SEQ ID NO: 12
  source 1..63: mol_type = other DNA

SEQ ID NO: 17 - DNA, 63 bp, Francisella tularensis; identical to SEQ ID NO: 11
  source 1..63: mol_type = other DNA

SEQ ID NO: 18 - DNA, 63 bp, Francisella tularensis; identical to SEQ ID NO: 12
  source 1..63: mol_type = other DNA

SEQ ID NO: 19 - DNA, 46 bp, Streptococcus pyogenes
  misc_feature 1: note = 5' /Alex647N/
  source 1..46: mol_type = other DNA
taatacgactcactataggacgcgaccgaaatggtgaaggacgggt

SEQ ID NO: 20 - DNA, 46 bp, Streptococcus pyogenes
  source 1..46: mol_type = other DNA
acccgtccttcaggttttcggtcgcgtcctatagtgagtcgtatta

SEQ ID NO: 21 - DNA, 16 bp, Streptococcus pyogenes
  misc_feature 16: note = 3' /IAbRQSp/ (Iowa Black RQ-Sp quencher)
  source 1..16: mol_type = other DNA
atagtgagtcgtatta

SEQ ID NO: 22 - DNA, 52 bp, Streptococcus pyogenes
  source 1..52: mol_type = genomic DNA
actcactatagggacgcgaccgaaatggtgaaggacgggtccagtgcttcgg

SEQ ID NO: 23 - DNA, 52 bp, Streptococcus pyogenes
  source 1..52: mol_type = other DNA
ccgaagcactggacccgtccttcaccatttcggtcgcgtccctatagtgagt

SEQ ID NO: 24 - DNA, 16 bp, Streptococcus pyogenes
  misc_feature 16: note = 3' /IAbRQSp/ (Iowa Black RQ-Sp quencher)
  source 1..16: mol_type = other DNA
cgtccctatagtgagt

SEQ ID NO: 25 - DNA, 51 bp, Francisella tularensis
  misc_feature 51: note = 3' /Alex647N/
  source 1..51: mol_type = other DNA
cgtccttcaccatttcggtcgcgtccctatagtgagtcgtattagttccat

SEQ ID NO: 26 - DNA, 51 bp, Francisella tularensis
  source 1..51: mol_type = other DNA
atggaactaatacgactcactatagggacgcgaccgaaatggtgaaggacg

SEQ ID NO: 27 - DNA, 16 bp, Francisella tularensis
  misc_feature 1: note = 5' /IABkFQ/
  source 1..16: mol_type = other DNA
atggaactaatacgac

SEQ ID NO: 28 - DNA, 51 bp, Francisella tularensis; identical to SEQ ID NO: 26
  misc_feature 1: note = 5' /Alex647N/
  source 1..51: mol_type = other DNA

SEQ ID NO: 29 - DNA, 51 bp, Francisella tularensis; identical to SEQ ID NO: 25
  source 1..51: mol_type = other DNA

SEQ ID NO: 30 - DNA, 16 bp, Francisella tularensis
  misc_feature 16: note = 3' /IABkFQ/
  source 1..16: mol_type = other DNA
gtcgtattagttccat

SEQ ID NO: 31 - DNA, 80 bp, Streptococcus pyogenes
  source 1..80: mol_type = other DNA
aaaagcaccgactcggtgccactttttcaagttgataacggactagccttattttaacttgctatttctagctctaaaac

SEQ ID NO: 32 - DNA, 54 bp, Streptococcus pyogenes
  source 1..54: mol_type = other DNA
taatacgactcactatagctataggacgcgaccgaaagttttagagctagaaat

SEQ ID NO: 33 - DNA, 54 bp, Streptococcus pyogenes
  source 1..54: mol_type = other DNA
taatacgactcactatagggcacgggcagcttgccgggttttagagctagaaat

SEQ ID NO: 34 - DNA, 54 bp, Streptococcus pyogenes
  source 1..54: mol_type = other DNA
taatacgactcactataggtccaggggtcttactcctgttttagagctagaaat

SEQ ID NO: 35 - DNA, 71 bp, Staphylococcus aureus
  source 1..71: mol_type = other DNA
aaaatctcgccaacaagttgacgagataaacacggcattttgccttgttttagtagattctgtttccagag

SEQ ID NO: 36 - DNA, 58 bp, Staphylococcus aureus
  source 1..58: mol_type = other DNA
taatacgactcactatagggacgcgaccgaaatggtgaagggttttagtactctggaa

SEQ ID NO: 37 - DNA, 25 bp, Francisella tularensis
  source 1..25: mol_type = other DNA
gaaattaatacgactcactataggg

SEQ ID NO: 38 - DNA, 69 bp, Francisella tularensis
  source 1..69: mol_type = other DNA
acgactcactatagggacgcgaccatctacaacagtagaaattaccctatagtgagtcgtattaatttc

SEQ ID NO: 39 - DNA, 20 bp, synthetic construct
  misc_feature 1..20: note = Synthetic
  source 1..20: mol_type = other DNA
gggcacgggcagcttgccgg

SEQ ID NO: 40 - DNA, 20 bp, synthetic construct
  misc_feature 1..20: note = Synthetic
  source 1..20: mol_type = other DNA
ggtccaggggtcttactcct

SEQ ID NO: 41 - DNA, 20 bp, synthetic construct
  misc_feature 1..20: note = Synthetic
  source 1..20: mol_type = other DNA
gagtccgagcagaagaagaa

SEQ ID NO: 42 - DNA, 20 bp, synthetic construct
  misc_feature 1..20: note = Synthetic
  source 1..20: mol_type = other DNA
gagtctaagcagaagaagaa

SEQ ID NO: 43 - DNA, 20 bp, synthetic construct
  misc_feature 1..20: note = Synthetic
  source 1..20: mol_type = other DNA
ggaatcccttctgcagcacc

SEQ ID NO: 44 - DNA, 20 bp, synthetic construct
  misc_feature 1..20: note = Synthetic
  source 1..20: mol_type = other DNA
ggaaccccgtctgcagcacc

SEQ ID NO: 45 - DNA, 20 bp, synthetic construct
  misc_feature 1..20: note = Synthetic
  source 1..20: mol_type = other DNA
gggtggggggagtttgctcc

SEQ ID NO: 46 - DNA, 20 bp, synthetic construct
  misc_feature 1..20: note = Synthetic
  source 1..20: mol_type = other DNA
cgggggagggagtttgctcc

SEQ ID NO: 47 - DNA, 183 bp, synthetic construct
  misc_feature 1..183: note = Synthetic
  source 1..183: mol_type = other DNA
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgc

SEQ ID NO: 48 - DNA, 57 bp, synthetic construct
  misc_feature 1..57: note = Synthetic
  variation 34..37: note = n is a, c, g, or t
  source 1..57: mol_type = other DNA
acactctttccctacacgacgctcttccgatctnnnnacgtaaacggccacaagttc

SEQ ID NO: 49 - DNA, 51 bp, synthetic construct
  misc_feature 1..51: note = Synthetic
  source 1..51: mol_type = other DNA
tggagttcagacgtgtgctcttccgatctgtcgtccttgaagaagatggtg

SEQ ID NO: 50 - DNA, 57 bp, synthetic construct
  misc_feature 1..57: note = Synthetic
  variation 34..37: note = n is a, c, g, or t
  source 1..57: mol_type = other DNA
acactctttccctacacgacgctcttccgatctnnnncagctcagcctgagtgttga

SEQ ID NO: 51 - DNA, 48 bp, synthetic construct
  misc_feature 1..48: note = Synthetic
  source 1..48: mol_type = other DNA
tggagttcagacgtgtgctcttccgatctctcgtgggtttgtggttgc

SEQ ID NO: 52 - DNA, 57 bp, synthetic construct
  misc_feature 1..57: note = Synthetic
  variation 34..37: note = n is a, c, g, or t
  source 1..57: mol_type = other DNA
acactctttccctacacgacgctcttccgatctnnnncacggcctttgcaaatagag

SEQ ID NO: 53 - DNA, 49 bp, synthetic construct
  misc_feature 1..49: note = Synthetic
  source 1..49: mol_type = other DNA
tggagttcagacgtgtgctcttccgatctggctttcacaaggatgcagt

SEQ ID NO: 54 - DNA, 57 bp, synthetic construct
  misc_feature 1..57: note = Synthetic
  variation 34..37: note = n is a, c, g, or t
  source 1..57: mol_type = other DNA
acactctttccctacacgacgctcttccgatctnnnncattgcagagaggcgtatca

SEQ ID NO: 55 - DNA, 47 bp, synthetic construct
  misc_feature 1..47: note = Synthetic
  source 1..47: mol_type = other DNA
tggagttcagacgtgtgctcttccgatctggggtcccaggtgctgac

SEQ ID NO: 56 - DNA, 59 bp, synthetic construct
  misc_feature 1..59: note = Synthetic
  variation 34..37: note = n is a, c, g, or t
  source 1..59: mol_type = other DNA
acactctttccctacacgacgctcttccgatctnnnngcgggcagtggcgtcttagtcg

SEQ ID NO: 57 - DNA, 51 bp, synthetic construct
  misc_feature 1..51: note = Synthetic
  source 1..51: mol_type = other DNA
tggagttcagacgtgtgctcttccgatctccctgggtttggttggctgctc

SEQ ID NO: 58 - DNA, 60 bp, synthetic construct
  misc_feature 1..60: note = Synthetic
  variation 34..37: note = n is a, c, g, or t
  source 1..60: mol_type = other DNA
acactctttccctacacgacgctcttccgatctnnnnggctctctgtacatgaagcaact

SEQ ID NO: 59 - DNA, 48 bp, synthetic construct
  misc_feature 1..48: note = Synthetic
  source 1..48: mol_type = other DNA
tggagttcagacgtgtgctcttccgatctcctagtgactgccgtctgc

SEQ ID NO: 60 - DNA, 57 bp, synthetic construct
  misc_feature 1..57: note = Synthetic
  variation 34..37: note = n is a, c, g, or t
  source 1..57: mol_type = other DNA
acactctttccctacacgacgctcttccgatctnnnnctcagcacctgcacttcttg

SEQ ID NO: 61 - DNA, 49 bp, synthetic construct
  misc_feature 1..49: note = Synthetic
  source 1..49: mol_type = other DNA
tggagttcagacgtgtgctcttccgatctcagatgtggccctgagagag

SEQ ID NO: 62 - protein, 13 aa, synthetic construct
  REGION 1..13: note = Synthetic
EHPVENTQLQNEK